SEQUENCE LISTING (1017 sequences)

SEQ ID NO: 1 - DNA, 14759 bp, Homo sapiens
  CDS (3639)...(3982)
  CDS (11755)...(12601)
ccttggttttggggatcatttgggcaagcccgaggtgctgtgcatgggggctcctggaatcctgggaagggcagaaagccttggccccagactcatcgtgcagcagctctgagcagtatttcggctgaggagtgacttcagtgaatattcagctgaggagtgacttggccacgtgtcacagccctacttcttgggggcctggtggaagagggtggcgtagaaggttccaaggtcccaaactggaattgtcctgtatgcttggttcacacagtgcgttattttaccttcctctgagctgctaatcgcctgcctctgagctgggtgagataaatatcacaaggcacaaagtgattgtacaataaaaaaatcaaatccctcccatccatccttcagtctgccacacacgcagtctacgttacacacatgtcacgtaaagcaggatgacatccatgtcacatacatagacatattaaccgaaatgtggcccttcggttgcatatattctcatacatgaatatatttatagaaatatatgcacatatttttgtatattggatatatttatgtaactataaatttacatgcgtatggatatgaaaataaatgcatacacatttatgtaaaaaaatttgtacacatgcatttacatatgtaaatacatacatctctatgtattaatgtttaaaaacactcaatttccagcctgctgttttcttttaattttcctcctattccggggaaacagaagcgtggatcccacgtctatgctatgccaaaatacgctgtaattgaggtgttttgttttgttttgttttttgaaatcgtatattaccgaaaaacttcaaactgaaagttgaataacgggcccagcggggaaataagaggccagaccctgaccctgcatttgtcctggatttcgcctccagagtccccgcgagggtccggcgcgccagctgatctctcctttgagagcagggagtggaggcgcgagcgccccccttggcggccgcgcgcccccgccctccgccccaccccgccgcggctgcccgggcgcgccgtccacacccctgcgcgcagctcccgcccgctcggggatccccggcgagccgcgccgcgaagggggaggtgttcggccgcggccgggagggagccggcaggcggcgtcccctttaaaagccgcgagcgccgcgccacggcgccgccgccgccgtcgccgccgccggagtcctcgccccgccgcgctgcgcccggctcgcgctgcgctagtcgctccgcttcccacaccccgccggggactggcagccgccgccgcacatctgccgccacagcctccgccggctacccgaacgttctcggggccagcgccgagtggatcaccggggaccgcgaggcacccgcgcgccgcagaccccgcgcgggctggagcacccggcagagcgcgccacagcgccgtggcctctgctgcccgggctgcgccagagccgcggacgggcgcgcagagcgccggggactccggagccgatccctagcgccgcgatgcggagcacctactgcaggagatcgggggcctgggacgcgctggccgaggtgtgatcggaccccaggctagccacaaagggcacttggccccagggctaggagagcgaggggagagcacagccacccgcctcggcggcccgggactcggctcgactcgccggagaatgcgcccgaggacgacggggcgccagagccgcggtgctttcaactggcgagcgcgaatgggggtgcactggagtaaggcagagtgatgcgggggggcaactcgcctggcaccgagatcgccgccgtgcccttccctggacccggcgtcgcccaggatggctgccccgagccatgggccgcggcggagctagcgcggagcgcccgaccctcgacccccgagtcccggagccggccccgcgcggggccacgcgtccctcgggcgctggttcctaaggaggacgacagcaccagcttctcctttctcccttcccttccctgccccgcactcctccccctgctcgctgttgttgtgtgtcagcacttggctggggacttcttgaacttgcagggagaataacttgcgcaccccactttgcgccggtgcctttgccccagcggagcctgcttcgccatctccgagccccaccgcccctccactcctcggccttgcccgacactgagacgctgttcccagcgtgaaaagagagactgcgcggccggcacccgggagaaggaggaggcaaagaaaaggaacggacattcggtccttgcgccaggtcctttgaccagagtttttccatgtggacgctctttcaatggacgtgtccccgcgtgcttcttagacggactgcggtctcctaaaggtagaggacgcgggccagggcccggggtgggtggtgggtgggagggggatttgggcagccactgcggtagagcccttccttacgtccaggccagaagtaaacagacccctctccagtccacgtgcaacggagccctgcaggggctcccacttccagctgccccgggcgaccgtaagcctcaccctcccggcccgcactcttccacccctctttcttcccctctccctggaatacttttggagctgttaacacttagatgaggtgttttatttatttatttatttatttttaatttttttaaaaacttttttgggtcaaagaaatccctttgagagggtagcccctgggtttcacccgttagctgagaacctgtccgctctgccatggtgatctccattcttcaagtgtttccgggagacttggtttctttgctcagagccgtgtcccatttaggaaagtactaggagtttggggttctccctacttgtttccagaaatgcgaggggtcagtactgaaggatcacttggtactgtgtttttaacagctgacacgtgcattaatagatattcaccatttacgtaatcccgggaagatacatgtgtatcttgactgcactgtggggatgcgggatggagctgcctttcgagacacccctgagggtaggggcctgggacacaagtcataagtggcttcagaagttgtggccttgagcttacagggtctggaagctataagggtgtgtgtgtgtgtgtgtgtgtgtgtgtgtgtgtgtcaggaagttctatacagtgcctctaaggaagtcacatgcaccatttatgtgtgtttatatgccagacagcgctcagcactccgcatttgggtttgtataggggacgcagggtgtcagatcaagcggtggttttcccaggttcccggcattggctgtcagcgctgtgtcacacacaaaaaagtgacagtcattggcgctggtttggttgggggggagggcaaatcccaaatctgatgtcagacgagctaagcgttggatgggagcgataaatcatctggttcaggaacttgggacccttcattatcccaaacgtttgagcttcggtcggtcttacctagactcgtgagtgtgccaagccaggagggcatcctggaggaggcacgccagccaaatgggagaccgggccgcgggggcgcgaggggggaggactgggcggggaactcgggtgactcacgtcggtcctgtccgcaggtcgaccatggtggccgggacccgctgtcttctagcgttgctgcttccccaggtcctcctgggcggcgcggctggcctcgttccggagctgggccgcaggaagttcgcggcggcgtcgtcgggccgcccctcatcccagccctctgacgaggtcctgagcgagttcgagttgcggctgctcagcatgttcggcctgaaacagagacccacccccagcagggacgccgtggtgcccccctacatgctagacctgtatcgcaggcactcaggtcagccgggctcacccgccccagaccaccggttggagagggcagccagccgagccaacactgtgcgcagcttccaccatgaaggtgaggcatggagcagggcgtgggggcggggagtcaccctgcaaagccctccaccgtgggcagactgcagccgtccctgtagaggcagcttggccggggcaccagcggacgtttccactcttgcttctgtactatcgtttctgaatctgattttaactcactgcttgtgtggtgggggagccagggattcccctttagtaactccgcaccctcttcctggcttgcagccagaagagctactcctcctggaagaattggagagaaatcaagtgatggggaagatgagggcaaaaggcatgcctctagtcagctaaacgtgcaagaattccacagagggaaaaggagaaaaagggaggcagattgagatttctttaagtctgtttggaagcttttgctctataaatctgccgcttaagccagggttttagggtagacagagccaagggcagagttttcagagatagtattgaaaaatcaaagcccagggccccaaagtctttctaatttatagttgatctgggcctggtttggaagattttgaatcccaatctaatccccgtgggagatcaatactacaatcaatcttattgtttccacaatgactttcttgtcctgtgcttaaatctgagataggctctgagtagagacaaggcaagccttcagataaaagcgtttgtagcagctgcctgtttttttttcatgtgcaccgaaatgtggatttttttttcttttatgatactacatgtggtttttctaaggtgggatatttctgcttgtttcatcagaagggcatttagtggactggaaatgtcttacagcagctattgaggtctgctgtacctaagttcttagagcaattagtcaaaaatatgttccacttcaattctttttctacacttttaaatgcttctttggcttaatacatttaaaatagagcatgggtttcttcaattcctagaaaagagtacaaaagtgtatatcacagagcaaccacttggcagatatttggggagttgggagtgaagttctctttcttgcctttccctgcttaggtggtaaatttcaagtgggaaatttacactgataatagactaatgggaaatggcacttccagatgttttctcccagtgtgaagggtgacttatacttgtgagagtatttgttggtaatgggaataagtcccaaaggcaagccacatagcagaagatacgttctcattgaggcagctacacattacgacggggacactgaattgatcatcagttcatttacaagcacatttctaagtgaggtgctctctgctagcagaaatcagatttgaaaggcagtaagatctcactccactctttcagaattcatccaatgaaagcagaaatcacctgttgtcatatgtaaaatttgtgtgtatgtgtacattctgccatcttaaccctgaaatgattatagatccagctaatcattcccaggtaatgctgattagaatacttttttttttgtataggaatgtaataagaacaactgttttagacacctcttctggaaatttagcatggaagctctcaactttatttttaaggcctggaagatgctgtgtctctgttacaacttaaaaggaagatcatttaagttagttaacacctaaaacattccattgtgtgaggattttatcagtgatgtctgcatattctcatcattcatctagaagtggtttgatcagaactaaacaggctacacgttattcaactgtgttattttaacttaaaaagcatgcttgagtttataaaatcagaatttatatctttgtgagtgtaaatgttacctgagaaacagtacagaagtgaccaacttgattaaaatcaacttgtaataacttcaggtcttaatgcagttagataatggagaaaagctatgtaattttgccccaaatttcaactaatccatttcttgtctcattatgactaatatatcatccttaatctggatggatatagcacttttttcaagactaatcattgttgtatacacccaggatttgcttttgataaacatccttgtgccatgcatgccacgaaaaaagtttttggtaaaccatgtgatgaaggttgctggctcaagaacagaatttagtttctacagcattaatgagcatttatttgaaaaaagaccataaagacccaatcataagaattacctgttgggttttctttgtaggtgtgatcgaatggtttggtggaattactcgacgagatatcatgatagcattctttcaaccaatatgagtataatgcgaccatatcataggggatctgagacagaattatcagttgtatttttcctattgaattttgtctagtcctttctccagtggcttttatttgggagaatatcagctttgctaaaatgttattgttttcaagatcattaaaaagtgcttcagctacatagacctttggaaactgccattgaacatagaaaagtcagttctgcaagtggaaagagtgttttgtgtattgctgtagttggaaacacattgaaactggttgacttcactggccctccaaaaagtctttatgcttttttgtcagatgggagagagaaagaccaggtgcttcttgttctcctcactctgaaggacacagtcttctttctacatgaaataactggattatttgcctctgtgactgaagctttcaaatagagattaaccctctttccacaaatataattattatgaaaatatccatataatagaaaagttcaagaaataactattgccctgcattagagactttgtggcacaaattcccccgtgcaaacaacagatttggacacatagatccaccaaaaccaatacttacctggtatggttccctagtggccccaggtatttcattgtcattacagaggccacattaagtaggaaaattactctatttggaaatggttgttgagattgaggctttggtgtccagtgatacttccttggcactgacattttccgttccacctgttttttagtggttcccctaaatttctcttaatccctttgcagtgaactattttgcgttcttagacttgctctttgtgtattttcactgagacaataagagaatatttcatcattccgaaggtgttggtgttaagggtgggcagaggccaaatcagggttgttgatgacaaccatgctctctattcctttatttgccattcccttgttgtattttttttaaaatggaatgtttttaaccttttgtatttgatattttttttctccttgatcagttgtctgttattttattatctggaaaatcttatattatactcagcctctttcattttgtgttagggcagtgacttccagccttactgattgccagcatatccccaggttttgttgttgttgttgttgttttactggagattttttagcccaaagtgtgttttaaaatcctcgaagcataacggtaacttacttttttgataaaacttaccatactttatttagaacaaaagggcagccacaaaatagcagtggctccttataaaatagacacattccagtgggccccgtcacttttctgctcatttctgtctgttctgtccatcatacctaagtcatatatttctgttcatttagttgggacagaactcacccaatgttatcattgtactaaatataaatgtgcccctaatggttttgacttttgcttaagtttttgagtcctcatgtatgttaggtagtgccatctagtagccagaaatttgggaactggctgggcatgatggctaatacctgtaatcccagcactttgggaggcttaggtgggtggatcacttgaggtcaggagttccagaccagcctggccaacatggtgaaaccacatctctactaaaatataaaaaaatagccaggtatgatggcccatgcctgtaatccgagctaattgggaggctgagatgggaggactgcttgaacctgggaggtggaggctgctgtgagccaagattgtgccactgcactccagcctgggcaacagagtgagaccctgtgtcacaaaaacaagaaacaaaacaaaacaaaagacaagaaacctgagaagcgcagtagattcaattatatatatctacttttaatttgctagctctgtgaccttaggaaagttacataacctctctgaactgcaactgtttcatttacaaaatggagataatgatagtttttctctaattggtttgttgtgagataattcatataaagctgatggtgccagattacactcaaaaaaagcattcagctgtcattatcattatgacttcttttgttaatgttatagcctttccttctctagggaaaaggaggccagagtggacctaggctgactgagagaattcagctcagtcttttgaattattttgaggtagaggaatgattgatatagtatagattattaaattaggacttcacttttggagaaaagttcagatatcattgttgtcttatttttcttcactttcccacatttttgcagccatagctccatccatttggttaagaacttagaagctcacaaactcgggtcaaagacaggtcgaaatcctcaaatcccttaagaacttcagcttattcaggaagggatatttacagaaaactagcaattgtataagtctccaaaaaagcatacattacttgaggatccatatatttttggcatcctcagggttgctgtgatgatttatagaaggtttgtttatttaatttactttatttcaaataggttttaatttttgtacccttaagaaaagattcgtactcttccctggcagattaaagaaaatgagcgtatattccctaaccttggccagttactttcctgggtttgagggtttctgtgaacgtctaacttacctctgtgacctgtttctgcaaccaggggtgttgcaatggatgcttttgtcttgaggatgggacctttcaagaaacagattcactgaggtgcagtgggaaggtcagagaaagatcttcgtatcgcctattattatttgctcgtctattttttctcctttcttaaggccactaactgattctcctttgctaaggctgcctacttccactgagaccttgaaccacatgaaattgttgttgtctgtgtttctggtcaaatagtggcaattttgtatgattcaatcttgtcatttaattttttgggaggttattattctatttcataccttttttatacccatcttctttacttcatttacctgtccctcatacttgacttgtagcttgtcccttcactgtcatcgtctggccatgtgggtgtgtacgtgtgtgcgagagagagaatgtgtgagaatgtatgtttctttatgcattgggatttagggtttttcttgcaattgtgatttctctgggcacttttgttaatatagctagtcagcgagtgctctagataattttccttgcctccccctctttgaaagaaaagagggtgttcttagatgtattcttatcagataagccagtagctcaggtgctggtctggctttggtgtcattggggtctgaggttgctgacttttaccttctctgctgaaaaattaccttcagcagaaacgtctgaattgcaaggagaaggagaaaaaaacaggccaaacacagtccttggtactccttgggagccactgagaagagtccaggttcaaatggtcagaaggttattttaatgattgtgtctggcctaaagtaccattagcttccagtggagtttagaatgtggatggatcctgaaaggtattccccagaggtttggattaataggcacaagggaaccctaaaggactctattggcctgatactccccatatccacgtagaagagctttagaagaaccttctgttctgagaccctggctgggcccacccagagctggcccattcaactcttactcctttgccaccactaatggttcttctactagtttttatattatttaacaaaaaggcactttaaaaatgcactcctggcaatctatactggaatatgaaaaacatgctgcaaaaccttgacactccaagtgtggtcttacagttcccagaatcccctccttgaggagctgctagaaatgctgaatctcaagcatctccccagacctactgaatcagagcctgcatctgaagctttacggtgtacaagctgttttatgtgaaggctgaagtttgaaaagcactgcattaaagcgttagtttggtataaactgccctgactgaacttggtgtgtccacttagcttgcatgatgactgttgctttgatgatgaaggcttacacgggtagatcctttgagtgagtgatctgacatgattctcctttgctaaggcatctagattcagtgcacaacttacagctgtttgtctttaggggaaatacaactgtaaaattaataaaaacatagtctcttcttatgataacatggaacgatggcaaaatagattttgttagcacttgggtaggaattctgaatgaagcaggcaaattctgttggcagtgaaatgataggatgtggtaaagttagaataaaataaacttaaatgtctcaaactctcatggtatatactaccagtttaataataatgttgtacctttgatgatttgcagactacaagcattcaaggtgctgtgttatatattacttgcttggagaataatacttcttaaaaattgaaattcagaaattttaaatcagacaaagcttttgtgcatggcccacttaaatggctattttgaaataatgatagtggatatagaaggattattctgtaataggatgagactgttccttttgtcatggagatcataatcatatttttgtaaatttttattatttttttggttttgtgtccatcctgcacactattactgggtaggtacatggttttttaacatggtttatctttcaaaactataaaggcattgcaaacagaagacaggtcatttatttttcttccaaaagcatctaaaatgagattttgatatttgaggtcataaagaggtgagagaacagacaacagttgggaaagctatttctcttgaaattgtttggccttaattactacagtgtcctagtaccacccatacgtttccaaagaagtagatccctgtaaatgcctttgtctctggacttttgagtaaaatagtagggtgtgctttgcaaaatgtcatcgttgatgttgagtttcagagtctttaattaggaagctgaaatctgtatatcgagatttgtaaatcatctaaattgcagagtaatgttttagaatactgcttaagggattggcattaaagccttttttaaaaaagaaatgcaataatttcctcaaatcctcactcattagacctctactaactatagtgctgacttttttttttttttaccctaaagtctggaattccaaagaaatgcttcaccatttcccccattattatagccacctggaagcagtattcatgtattagatcaaaaacacaacaaagaattatgaaaggttgtttcctggtatgcaatgcatgatgacatgaacttacagaacagagagaagggaggctccatgtttatttaaagaggaaatttttattttctggttacctacttttacatgggttacatcaaatcccacgatgaggtttaaaaattctcatagataatcaaacgtcattacttggcttactgaaattcagacttttcttttttcttccctgtttttctctatcaaattagaatctttggaagaactaccagaaacgagtgggaaaacaacccggagattcttctttaatttaagttctatccccacggaggagtttatcacctcagcagagcttcaggttttccgagaacagatgcaagatgctttaggaaacaatagcagtttccatcaccgaattaatatttatgaaatcataaaacctgcaacagccaactcgaaattccccgtgaccagacttttggacaccaggttggtgaatcagaatgcaagcaggtgggaaagttttgatgtcacccccgctgtgatgcggtggactgcacagggacacgccaaccatggattcgtggtggaagtggcccacttggaggagaaacaaggtgtctccaagagacatgttaggataagcaggtctttgcaccaagatgaacacagctggtcacagataaggccattgctagtaacttttggccatgatggaaaagggcatcctctccacaaaagagaaaaacgtcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaactctgttaactctaagattcctaaggcatgctgtgtcccgacagaactcagtgctatctcgatgctgtaccttgacgagaatgaaaaggttgtattaaagaactatcaggacatggttgtggagggttgtgggtgtcgctagtacagcaaaattaaatacataaatatatatatatatatatattttagaaaaaagaaaaaaacaaacaaacaaaaaaaccccaccccagttgacactttaatatttcccaatgaagactttatttatggaatggaatggaaaaaaaaacagctattttgaaaatatatttatatctacgaaaagaagttgggaaaacaaatattttaatcagagaattattccttaaagatttaaaatgtatttagttgtacattttatatgggttcaaccccagcacatgaagtataatggtcagatttattttgtatttatttactattataaccactttttaggaaaaaaatagctaatttgtatttatatgtaatcaaaagaagtatcgggtttgtacataattttccaaaaattgtagttgttttcagttgtgtgtatttaagatgaaaagtctacatggaaggttactctggcaaagtgcttagcacgtttgcttttttgcagtgctactgttgagttcacaagttcaagtccagaaaaaaaaagtggataatccactctgctgactttcaagattattatattattcaattctcaggaatgttgcagagtgattgtccaatccatgagaatttacatccttattaggtggaatatttggataagaaccagacattgctgatctattatagaaactctcctcctgccccttaatttacagaaagaataaagcaggatccatagaaataattaggaaaacgatgaacctgcaggaaagtgaatgatggtttgttgttcttctttcctaaattagtgatcccttcaaaggggctgatctggccaaagtattcaataaaacgtaagatttcttcattattgatattgtggtcatatatatttaaaattgatatctcgtggccctcatcaagggttggaaatttatttgtgttttacctttacctcatctgagagctctttattctccaaagaacccagttttctaactttttgcccaacacgcagcaaaattatgcacatcgtgttttctgcccaccctctgttctctgacctatcagcttgcttttctttccaaggttgtgtgtttgaacacatttctccaaatgttaaacctatttcagataataaatatcaaatctctggcatttcattctataaagtccaacctgtaagagaaaatggtgcatttgtatagcgcttacaatgatgaccttgtgtttgcatttttgtttctgaagttatatattttagagggggtgggggaaaggtaatgaatggctggaaaattgcaggcaagttatttgataagtcatatttgcactaaaggtgttaccagtgatttagtatttttcaaatgaacttctttggggcagaaagatttaagggaaaactaaagcctacaaaacaagcaaaacctggataacccgagataaagtttcagagataatagcccatgcaacagaggcaacggtgccagaaaattagaaagggaaagtgtcggagatcagcttctataagaacatctgccagttggactgacgcccaaacagaatgaagtcaaattaggctgctcagattgaacacttaccagagtgtcagggcttctgtaccctgggttagaatcagaccaaggaagggttcagcagatgttcataagagcagggcacccacaactacccactattttactggcagtattttaggtcagtttccaggactttgcatcccctctgatcctgccatgcatgattggtgaaacctacctctaatctccttggaattggctaaaaaacagtgtgtttataatggaacagactgttataatcaaattcttcctaggaattaacttttgatgactatgagcttagttacagttcggaggttatgaggttatgtaaaccttatctttaaatgtgcatgacagttatcttttactaatgctggttaacttttaaaatcttgcagctcctttttatctctagttctattgttcttgattaggtgagaaccattagatcatacccaactgaggggattggggtcttgtttgttctccagctgttcttcaccctctattgccatggacatgaaggacagactgcacggtcttaacatgttaaaacgaatgacccatgttttctcatat

SEQ ID NO: 2 - protein, 396 aa, Homo sapiens
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 3 - protein, 281 aa, Homo sapiens
ESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 4 - protein, 379 aa, Homo sapiens
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEK

SEQ ID NO: 5 - protein, 396 aa, Homo sapiens; identical to SEQ ID NO: 2

SEQ ID NO: 6 - protein, 240 aa, Homo sapiens [sequence omitted]

SEQ ID NO: 7 - protein, 396 aa, Artificial Sequence; identical to SEQ ID NO: 2
  consensus sequence

SEQ ID NO: 8 - DNA, 25 bp, Artificial Sequence
  amplification primer
atcacgccttttactgccacggaga

SEQ ID NO: 9 - DNA, 26 bp, Artificial Sequence
  amplification primer
atactttggccagatcagcccctttg

SEQ ID NO: 10 - DNA, 24 bp, Artificial Sequence
  amplification primer
tgatcccttcaaaggggctgatct

SEQ ID NO: 11 - DNA, 24 bp, Artificial Sequence
  amplification primer
tgatcccttcaaaggggctgatct

SEQ ID NO: 12 - DNA, 24 bp, Artificial Sequence
  amplification primer
tgatcccttcaaaggggctgatct

SEQ ID NO: 13 - DNA, 24 bp, Artificial Sequence
  amplification primer
tgatcccttcaaaggggctgatct

SEQ ID NO: 14 - DNA, 25 bp, Artificial Sequence
  amplification primer
caaaggggctgatctggccaaagta

SEQ ID NO: 15 - DNA, 20 bp, Artificial Sequence
  amplification primer
atcatgccattgttcagacg

SEQ ID NO: 16 - DNA, 21 bp, Artificial Sequence
  amplification primer
tgttcaaacacacaaccttgg

SEQ ID NO: 17 - DNA, 26 bp, Artificial Sequence
  amplification primer
tgttcccagcgtgaaaagagagactg

SEQ ID NO: 18 - DNA, 20 bp, Artificial Sequence
  amplification primer
ctttcccccaccccctctaa

SEQ ID NO: 19 - DNA, 26 bp, Artificial Sequence
  amplification primer
tgttcccagcgtgaaaagagagactg

SEQ ID NO: 20 - DNA, 25 bp, Artificial Sequence
  amplification primer
tctccgtggcagtaaaaggcgtgat

SEQ ID NO: 21 - DNA, 214 bp, Homo sapiens
  misc_feature 161: n = A,T,C or G
  misc_feature 161: n = A,T,C or G
  misc_feature 161: n = A,T,C or G
tatcacgccttttactgccacggagaatgcccttttcctctggctgatcatctgaactccactaatcatgccattgttcagacgttggtcaactctgttaactctaagattcctaaggcatgctgtgtcgcgacagaactcagtgctatctcgatgctgtnccttgacgagaatgaaaagtgatcccttcaaaggggcgatctggccaaagtat

SEQ ID NO: 22 - DNA, 351 bp, Homo sapiens
tgatcccttcaaaggggctgatctggccaaagtattcaataaaacgtaagatttcttcattattgatattgtggtcatatatatttaaaattgatatctcgtggccctcatcaagggttggaaatttatttgtgttttacctttacctcatctgagagctctttattctccaaagaacccagccaacacgcagcaaaattatgcacatcgtgttttctgcccaccctctgttctctgacctatcagcttgcttttctttccaaggttgtgtgtttgaacacatttctccaaatgttaaacctatttcagataataaatatcaaatctctggcatttcattctataaagtcc

SEQ ID NO: 23 - DNA, 341 bp, Homo sapiens
tgatcccttcaaaggggctgatctggccaaagtattcaataaaacgtaagatttcttcattattgatattgtggtcatatatatttaaaattgatatctcgtggccctcatcaagggttggaaatttatttgtgttttacctttacctcatctgagagctctttattctccaaagaacccagccaacacgcagcaaaattatgcacatcgtgttttctgcccaccctctgttctctgacctatcagcttgcttttctttccaaggttgtgtgtttgaacacatttctccaaatgttaaacctatttcagataataaatatcaaatctctggcatttcattc

SEQ ID NO: 24 - DNA, 66 bp, Homo sapiens
tgatcccttcaaaggggctgatctggccaaagtattcaataaaacgtaagatttcttcattattga

SEQ ID NO: 25 - DNA, 65 bp, Homo sapiens
tgatcccttcaaaggggctgatctggccaaagtattcaataaaacgtaagatttcttcattattg

SEQ ID NO: 26 - DNA, 560 bp, Homo sapiens
  misc_feature 532, 535: n = A,T,C or G
  misc_feature 532, 535: n = A,T,C or G
  misc_feature 532, 535: n = A,T,C or G
cccttcaaaggggctgatctggccaaagtattcaataaaacgtaagatttcttcattattgatattgtggtcatatatatttaaaattgatatctcgtggccctcatcaagggttggaaatttatttgtgttttacctttacctcatctgagagctctttattctccaaagaacccagttttctaactttttgcccaacgcgcagcaaaattatgtacatcgtgttttctgcccaccctctgttctctgacctatcagcttgcttttctttccaaggttgtgtgtttgaacacatttctccaaatgttaaacctatttcagataataaatatcaaatctctggcatttcattctataaagtccaacctgtaagagaaaatggtgcatttgtatagcgcttacaatgatgaccttgtgtttacatttttgtttctgaagttatatattttagagggggtgggggaaaggtagtgaatggctggaaaattgcaggcaagttatttgataagtcatatttgcactaaaggtgttnccngaaaaaaaaaaaaaaaaaaaaaaaa

SEQ ID NO: 27 - DNA, 412 bp, Homo sapiens
atcatgccattgttcagacgttggtcaactctgttaactctaagattcctaaggcatgctgtgtcccgacagaactcagtgctatctcgatgctgtaccttgacgagaatgaaaagtgatcccttcaaaggggctgatctggccaaagtattcaataaaacgtaagatttcttcattattgatattgtggtcatatatatttaaaattgatatctcgtggccctcatcaagggttggaaatttatttgtgttttacctttacctcatctgagagctctttattctccaaagaacccagttttctaactttttgcccaacacgcagcaaaattatgcacatcgtgttttctgcccaccctctgttctctgacctatcagcttgcttttctttccaaggttgtgtgtttgaaca

SEQ ID NO: 28 - DNA, 899 bp, Homo sapiens
  misc_feature 3, 5, 688, 763: n = A,T,C or G
  misc_feature 3, 5, 688, 763: n = A,T,C or G
  misc_feature 3, 5, 688, 763: n = A,T,C or G
tcntnggcgatgggcctctagatgctgctcgagcggccgccagtgtgatggatatctgcagaattcgccctttgttcccagcgtgaaaagagagactgcgcggccggcacccgggagaaggaggaggcaaagaaaaggaacggacattcggtccttgcgccaggtcctttgaccagagtttttccatgtggacgctctttcaatggacgtgtccccgcgtgcttcttagacggactgcggtctcctaaagaatctttggaagaactaccagaaacgagtgggaaaacaacccggagattcttctttaatttaagttctatccccacggaggagtttatcacctcagcagagcttcaggttttccgagaacagatgcaagatgctttaggaaacaatagcagtttccatcaccgaattaatatttatgaaatcataaaacctgcaacagccaactcgaaattccccgtgaccagtcttttggacaccaggttggtgaatcagaatgcaagcaggtgggaaagttttgatgtcacccccgctgtgatgcggtggactgcacagggacacgccaaccatggattcgtggtggaagtggcccacttggaggagaaacaaggtgtctccaagagacatgttaggataagcaggtctttgcaccaagatgaacacagctggtcacagataaggncattgctagtaacttttggccatgatggaaaagggcatcctctccacaaaagagaaaaacgtcaagccaaacacnaacagcggaaacgccttaagtccagctgtaagagacacctttgtacgtggacttcagtgacgtggggtggaatgactggattgtggctccccccggggtatcacgccttttactgccacggagaaagggggaattc

SEQ ID NO: 29 - DNA, 515 bp, Homo sapiens
  misc_feature 2, 14, 55, 65, 68, 73, 83, 94, 95, 112, 126, 279, 281,510: n = A,T,C or G
  misc_feature 2, 14, 55, 65, 68, 73, 83, 94, 95, 112, 126, 279, 281,510: n = A,T,C or G
  misc_feature 2, 14, 55, 65, 68, 73, 83, 94, 95, 112, 126, 279, 281,510: n = A,T,C or G
antggggcctctanatgcatgctcgaagcgggcccccagtgtggatgggatattntggaagaatnccnttttntgttcccagnggtgaaaagannaaactgcgcgggccggncacccggggagaanggaggaggcaaagaaaaggaacggacattcggtccttgcgccaggtcctttgaccagagtttttccatggtggacgctctttcaatggacgtgtccccgcgtgcttcttagacggactgcggtctcctaaaggtcgaccatgggtgggcgggnanccgctgtcttctggcgttgctgcttccccaggtcctcctgggcggcgcggctggcctcgttccggagctgggccgcaggaagttcacggcggcgtcgtcgggccgcccctcatcccagccctctgacgaggtcctgagcgagttcgagttgcggccgctcagcatgttcggcctgaaacagagacccacccccagcagggacggccgtgggtgcccccctacatgctanaacct

SEQ ID NO: 30 - DNA, 881 bp, Homo sapiens
  misc_feature 2, 11, 615, 656, 701, 746, 752, 823, 848, 858: n = A,T,C or G
  misc_feature 2, 11, 615, 656, 701, 746, 752, 823, 848, 858: n = A,T,C or G
  misc_feature 2, 11, 615, 656, 701, 746, 752, 823, 848, 858: n = A,T,C or G
tngggacttanatctcagctatgcatcaagcttggtaccgagctcggatccactagtaacggccgccagtgtgctggaattcgcccttctttcccccaccccctctaaaatatataacttcagaaacaaaaatgcaaacacaaggtcatcattgtaagcgctatacaaatgcaccattttctcttacaggttggactttatagaatgaaatgccagagatttgatatttattatctgaaataggtttaacatttggagaaatgtgttcaaacacacaaccttggaaagaaaagcaagctgataggtcagagaacagagggtgggcagaaaacacgatgtgcataattttgctgcgtgttgggcaaaaagttagaaaactgggttctttggagaataaagagctctcagatgaggtaaaggtaaaacacaaataaatttccaacccttgatgagggccacgagatatcaattttaaatatatatgaccacaatatcaataatgaagaaatcttacgttttattgaatactttggccagatcagcccctttgaagggatcactaatttaggaaagaagaacaacaaaccatcattcactttcctgcaggttcatcgntttcctaattatttctatggatcctgctttattctttctgnaaattaaggggcaggacgagagtttctataatagatcagcaatgnctggttcttatccaaatattcacctaataaggatgtaaattctcntggatnggacaaccactctgcaacattcctgagaatgaataatataataatcttgaagtcagcagagtggatatccncttttttttttctggacttgaactntgaactcacngagcactgcaaaaaagcaacgtg

SEQ ID NO: 31 - DNA, 912 bp, Homo sapiens
  misc_feature 2, 22, 34, 534, 754, 897, 903: n = A,T,C or G
  misc_feature 2, 22, 34, 534, 754, 897, 903: n = A,T,C or G
  misc_feature 2, 22, 34, 534, 754, 897, 903: n = A,T,C or G
cnctctgggggcggtgggctcnttcggactgggncgcaggaattcgcggcggcgtcgtcgggccgcccctcatcccagccctctgacgaggtcctgagcgagttcgagttgcggctgctcagcatgttcggcctgaaacagagacccacccccagcaggggacgccgtggtgcccccctacatgctagacctggtatcgcaggcactcgggtcagccgggctcacccgccccagaccaccggttggagagggcagccagccgagccaacactgtgcgcagcttccaccatgaagaatctttggaagaactaccagaaacgagtgggaaaacaacccggagattcttctttaatttaagttctatccccacggaggagtttatcacctcagcagagcttcaggttttccgagaacagatgcaagatgctttaggaaacaatagcagtttccatcaccgaattaatatttatgaaatcataaaacctgcaacagccaacgcgaaattccccgtgaccagtcttttggacaccaggntggtgaatcagaatgcaagcaggtgggaaagttttgatgtcacccccgctgtgatgcggggggactgcacagggacacgccaaccatggattcgtggggggaagtggcccacttggaggagaaacaaggtgtctccaagagacatgttaggataagcaggtctttgcaccaagatgaacacagctggtcacagataagggcattgctagtaacttttggncatgatggaaaagggcatcctctccacaaaagagaaaaacgtcaagccaaacacaaacagcggaaacgccttaagtccagctgtaagagacaccctttgtacgtggacttcagtgacgtgggggggaatgactggattgtggntccccnggggatcac

SEQ ID NO: 32 - DNA, 809 bp, Homo sapiens
  misc_feature 7, 8, 28, 68, 144, 735, 751: n = A,T,C or G
  misc_feature 7, 8, 28, 68, 144, 735, 751: n = A,T,C or G
  misc_feature 7, 8, 28, 68, 144, 735, 751: n = A,T,C or G
gccgggnncgccttttctgcccggagangcccttttcctctggctgatcatctgaactccactaatcntgccattgttcagacgttggtcaactctgttaactctaagattcctaaggcatgctgtgtcccgacagaactcagntgctatctcgatgctgtaccttgacgagaatgaaaaggttgtattaaagaactatcaggacatggttgtggagggttgtgggtgtcgctagtacagcaaaattaaatacataaatatatatatatattttagaaaaaagaaaaaaacaaacaaacaaaaaaaccccaccccagttgacactttaatatttcccaatgaagactttatttatggaatggaatggaaaaaaaacagctattttgaaaatatatttatatctacgaaaagaagttgggaaaacaaatattttaatcagagaattattccttaaagatttaaaatgtatttagttgtacattttatatgggttcaaccccagcacatgaagtataatggtcagatttattttgtatttatttactattataaccactttttaggaaaaaaatagctaatttgtatttatatgtaatcaaaagaagtatcgggtttgtacataattttccaaaaattgtagttgttttcaggtgtgtgtatttaagatgaaaagtctacatggaaggttactctggcaaagtgcttagcacgtttgcttttttgcagtgctactgntgagttcacaagttcnagtccagaaaaaaaagtggataatccactctgctgactttcaagattattatattatt

SEQ ID NO: 33 - protein, 380 aa, Homo sapiens
PMVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEK

SEQ ID NO: 34 - protein, 273 aa, Homo sapiens
  VARIANT 207, 232: Xaa = Any Amino Acid
  VARIANT 207, 232: Xaa = Any Amino Acid
  VARIANT 207, 232: Xaa = Any Amino Acid
PCSQREKRDCAAGTREKEEAKKRNGHSVLAPGPLTRVFPCGRSFNGRVPACFLDGLRSPKESLEELPETSGKTTRRFFFNLSSIPTEEFITSAELQVFREQMQDALGNNSSFHHRINIYEIIKPATANSKFPVTSLLDTRLVNQNASRWESFDVTPAVMRWTAQGHANHGFVVEVAHLEEKQGVSKRHVRISRSLHQDEHSWSQIRXLLVTFGHDGKGHPLHKREKRQAKHXQRKRLKSSCKRHLCTWTSVTWGGMTGLWLPPGYHAFYCHGE

SEQ ID NO: 35 - DNA, 202 bp, Homo sapiens
ctgtggtgtgtccattagggacatgacaacagccattatgtaagtaaaccatagagggtctgatttctcacttgatcttgtatattatttctctttctttagtgttagctcttctgcacctgtgtttgagttaactcttggctctgaggacatttaggggatgattagaatatttaaaaccagcgagtaaacagatctcata

SEQ ID NO: 36 - DNA, 198 bp, Homo sapiens
aaagaacaatataaataagcacattttcaaaagttattaaaaagaagaggcaatgagagttagagacatttgaggaagttgtagaggaagaaaaggaacaactcagtgggggaagtggtgagaccaaatgtgctgaggctgaaatggccttggtgtgcttgggagacagtgagtacgtgaatctgcctgcagaagttg

SEQ ID NO: 37 - DNA, 200 bp, Homo sapiens
tgcagaagttgggagtggggggaatattattaggattattaggaaggtaagctgagaacataatgtcaagagtcttaaatgctggctaataagtatcttatgaaagttacttgagcaggaaagtgatgcaataaggtcctatttaattgacagctgattatagaatggactggcaggggagtgtctagaagtaatatcaa

SEQ ID NO: 38 - DNA, 202 bp, Homo sapiens
acaatgtgtctttttcctctggctgcctttaatattttctctttatatgcgattttcagcagtttgaatatgatttccccagcattgtgtttgtgtatagagtgtgtgagtgtatgttggtatttatcttgctagatgttctctgagcttattgagtctgttttttggtgtcttatatcagttttggaaaatactcagggaa

SEQ ID NO: 39 - DNA, 202 bp, Homo sapiens
atttatcttgctagatgttctctgagcttattgagtctgttttttggtgtcttatatcagttttggaaaatactcagggaatagttactcaaaaaattttataaaaatctaaaatttatggtgacctcagattggcattcctcccaagtatctggtgttagcaaactcaaatattatctggaaaaaggacattttatcaggc

SEQ ID NO: 40 - DNA, 202 bp, Homo sapiens
agtaggtcatcagtaggtacaaaggatagattgtgaccaacaactttgtgtagataaagtggttctcccagcagttgatgttgagatttcattaaggaaaagtgcatcatttaaacaaaaatagtctgaggtctgtctggttcaagataatggacaacgtggaaatattctcatctccttctctgaacctattaaaatggca

SEQ ID NO: 41 - DNA, 202 bp, Homo sapiens
tgcaaatgacatcataacttttcttcatgggtgagatatcctgagattgctattagagaaaataacgatacttgtcatatatttcctctaaataaatctggtctttaatagcaaaagtccatgttttcttcacttctgtttatttgcagtcactcaaactgacacgtgctgtcaatatctcctcactgtaagtgtaagcaga

SEQ ID NO: 42 - DNA, 201 bp, Homo sapiens
tataattcatgccagactatatattactgtctccctcttcctccccaaaaatcgcatgtcacacattaccttgtgtaaataggcatttgagattcttcaacttaactttgacagtatgtttgcagtaacattctcttcaattgcctctgtacctgtagggcaaagttcagcatcatcctctctgctgaaataaccaacatg

SEQ ID NO: 43 - DNA, 202 bp, Homo sapiens
cattaattggtcatgtgagcactaaggataaccaccctcccctcctcaatcaggtaaccctaattattttctcttggtaaaagcacactttgctatgtcacttttcattgcgacaaacacagacttgtgctgagggaaatgccttttatgtgggaaccattaaaactgttagatgcagaaattccagcaatttagcttttaa

SEQ ID NO: 44 - DNA, 202 bp, Homo sapiens
gaaacttctgaaagacgtgagcctgtcatgttaagacacacagactatgccatgttgtttgggagaaaattgttgaaagttagtggtaacaatgtttccactgctcatcattctcagggttaagggaggagttattaaaaatctactcaattctgaaataaggctcattctaggtgtaatgcatacgatttgatgaatgtgg

SEQ ID NO: 45 - DNA, 202 bp, Homo sapiens
ccatgttgtttgggagaaaattgttgaaagttagtggtaacaatgtttccatgctcatcattctcagggttaagggaggagttattaaaaatctactcaacttctgaaataaggctcattctaggtgtaatgcatacgatttgatgaatgtggaagcccaaaaacttggcttccgtgacaacttaaatcagaagaaatgaac

SEQ ID NO: 46 - DNA, 201 bp, Homo sapiens
ggagttattaaaaatctactcaattctgaaataaggctcattctaggtgtaatgcatacgatttgatgaatgtggaagcccaaaaacttggcttccgtgaacaacttaaatcagaagaaatgaacttcatttcgtacacggttttcttccatcattactcataaaaaagaatttcaaggttggatgatttcttctatctgt

SEQ ID NO: 47 - DNA, 202 bp, Homo sapiens
gatgaatgtggaagcccaaaaacttggcttccgtgacaacttaaatcagaagaaatgaacttcatttcgtacacggttttcttccatcattactcataaaacaagaatttcaaggttggatgatttcttctatctgttcaccttctatccataatattcccgtcaggtggttagatgcctgtatcttgactacctgcagcaa

SEQ ID NO: 48 - DNA, 202 bp, Homo sapiens
taagtcatctactaatttggaaaccatgctgctttataggcattatacatagtgtctttgtgatatatttcacttcagtgtttccatttgaaaccaatatagaaagtcacactttttcaaaataagtataagaaaaaaattagacaatattccaaggatatgcttgatatgttgtgtggagagttcagacaacatgtgacct

SEQ ID NO: 49 - DNA, 202 bp, Homo sapiens
tggatttcaagctgggtgaaatgactttttttgcttgtaaagaagaaatgatgatggaggtctcatcaggaagtgatgcagaagactgggacaggtgagtagagcaccactggcagagttaattctggctgccatgcaactccgtagtaagtgtcagtagcaaagctgaagtcaaacattgtttactcctctgctaaggctc

SEQ ID NO: 50 - DNA, 202 bp, Homo sapiens
tggacttacaaatgaggctgctctgtgtgtggtggggggatgggtggttctcagtgattctttcatttttcatcaggtcctagacctcagtcttcacctcctggccccaggccctttgttcccctcacctcagtctcagaaatggtcacattctacagttcttgatgatgagctttctctggagcaatgggggtacccgcca

SEQ ID NO: 51 - DNA, 202 bp, Homo sapiens
tctgtgtgtggtggggggatgggtggttctcagtgattctttcatttttcatcaggtcctagacctcagtcttcacctccggccccaggccctttgttccctctcacctcagtctcagaaatggtcacattctacagttcttgatgatgagctttctctggagcaatgggggtacccgccaaaccctttcttctccaatgaa

SEQ ID NO: 52 - DNA, 202 bp, Homo sapiens
cctccggccccaggccctttgttcccctcacctcagtctcagaaatggtcacattctacagttcttgatgatgagctttctctggagcaatgggggtaccctgccaaaccctttcttctccaatgaaacctgtgtgtccaagacaaggaaacctcacacactgctttgtgaactagacaagaagggaataggtgagcagacg

SEQ ID NO: 53 - DNA, 202 bp, Homo sapiens
aggaaacctcacacactgctttgtgaactagacaagaagggaataggtgagcagacgctgtagcaaagtgactctgccctggtgtgagatggtgggaaagcggggtagtctgagcctgggcttagctttaactctgagggtaagggcctgcaccccaggcatacacagcagtcatctgttctctcttctgtgtatttacttc

SEQ ID NO: 54 - DNA, 202 bp, Homo sapiens
atctggagcaagactagctggcagctcaggaggaggcagcctcagctagcaaaagaaagcgtgttggagggttggggcccaggagagtcaatataatgaactctctttgcttgtttccagttccctgtctattagctcctatattcactgtctggcaggtaggttgtacttttggattaagttcttcttttgaaccttatct

SEQ ID NO: 55 - DNA, 202 bp, Homo sapiens
tctgtttgctcaggtttccaaattgctctgggtgacatctgcttgtcaccagtaaaaccaaaaggacagcagggtggattgccttgcacagggcaggcagagtaattgcagctttaattttgtttcgtttttggaaggatccaggtgtccaacctctttatcgcacaaaagaaccttaataaatgtgggagtgttggcagac

SEQ ID NO: 56 - DNA, 202 bp, Homo sapiens
caacagcttccaagctaggagcacaattagagacatggagacttctaagcagccaagagatacatttcaccctggaaattcagacctcagcagagaatacgtcaggccgtgtgaacataaatcattcccctgaaaagtctttcttcttgggaaacaaaattccccagagttggaatctgttggatcgttggtaggtgtgtct

SEQ ID NO: 57 - DNA, 202 bp, Homo sapiens
ttggaaggaacaggtggacttagctctggataagtcaggggactagctgaaactagatacgggtgtcaaataaaagatttctggaacttgggcttaatcacgggcctgcctgtgcagcctcagtgaaaacaaaatagtaggaatccaaagatgaagtagactagaggatcagcaagggctccaaagaaagagtatttggtga

SEQ ID NO: 58 - DNA, 202 bp, Homo sapiens
gataagtcaggggactagctgaaactagatacgggtgtcaaataaaagatttctggaacttgggcttaatcagggcctgcctgtgcagcctcagtgaaaactaaaatagtaggaatccaaagatgaagtagactagaggatcagcaagggctccaaagaaagagtatttggtgaatctatggtcaggctttagtggttcaga

SEQ ID NO: 59 - DNA, 202 bp, Homo sapiens
taggaaaaaaaatgaactgattatgtttcactatttatcatcatctttctagtttggctgggtggcagcctgatgatatttaaactgtgttggcataaaggtcaactcatagttttcttgccacttgggtacattttcagtgggcttgggggaggttgaactttagtaaatacacagtttcaacagctgtattgtctgaggg

SEQ ID NO: 60 - DNA, 202 bp, Homo sapiens
gttttcttgccacttgggtacattttcagtgggcttgggggaggttgaactttagtaaatacacagtttcaacagctgtattgtctgagggtattaaaatagtgtaggccaggtgtggcggctcatgcctgtaatcccagcaatctgggaggctgaggcaggtggatcacttgagatcaggagttccagcagcctggccaac

SEQ ID NO: 61 - DNA, 202 bp, Homo sapiens
agagcgtatgttgaaatcagcataaccctcttaggctttcaaaacgcaacctcaagaggccaagagatgtgttggcagcacgagcacttggctgtttgtactggcgatcatctcccccatttacccaaatatgttctgggcattttgtttacccctcacattgtctgtctgtgcgctttaacagatcaaatggcacctatta

SEQ ID NO: 62 - DNA, 202 bp, Homo sapiens
acctcaagaggccaagagatgtgttggcagcacgagcacttggctgtttgtatggcgatcatctcccccatttacccaaatatgttctgggcattttgttgtacccctcacattgtctgtctgtgcgctttaacagatcaaatggcacctattagtatgttgtggcctctttccagagggactcattaccgcctttactgct

SEQ ID NO: 63 - DNA, 202 bp, Homo sapiens
gacaaaggagacagtggcttttatcacaggcatattattctgccaccgaggctgtccttgcatctgttggcacaacaccacagaaagtaaaacaggctccctggaatggagttatttccatcccctcccacatagtattccctctggagcaagcaatttgtctatactcaacttttccctgttgattctctgaatcagacag

SEQ ID NO: 64 - DNA, 202 bp, Homo sapiens
gcacaacaccacagaaagtaaaacaggctcccggaatggagttatttccatcccctcccacatagtattccctctggagcaagcaatttgtctatactcaagcttttccctgttgattctctgaatcagacagactttccccaaacccctagagctattagccttgttaatctttgcccttcacaaatgaatcttttgtgct

SEQ ID NO: 65 - DNA, 202 bp, Homo sapiens
tttggaggattccgttgtcaagggacacagggcagggttacctcgtaaggaaataatgtggtcatgtgatgaagagctaaggctgagagaaactgcaaggatttctgctgctgaacaatgacaaagttcactggtgttctcaggtgcaagggagagaataggccctcattgtaaggcagcatcagccctgcctttgtttctg

SEQ ID NO: 66 - DNA, 202 bp, Homo sapiens
cagaacactgcaacagaaggtgacttggagaagggaaacttggaccacctcctcctcctccccactccatggggagaatggagtgaatccccaaatatacaggagggtaggggcaaaggaatttgagtttaggacatccttttaaatgttgtgagtttggacatcctttttaaacgctgtggagcagggtaaaacctgctgt

SEQ ID NO: 67 - DNA, 202 bp, Homo sapiens
gagaatggagtgaatccccaaatatacggagggtaggggcaaaggaatttgagtttaggacatccttttaaatgttgtgagtttggacatcctttttaaactgctgtggagcagggtaaaacctgctgtctccgcgacgttgggggttatccgaaggagacctttgctgtgtatttgtactgaaccatcattttctttattt

SEQ ID NO: 68 - DNA, 202 bp, Homo sapiens
ggtaggggcaaaggaatttgagtttaggacatccttttaaatgttgtgagtttggacatcctttttaaacgctgtggagcagggtaaaacctgctgtctcctgcgacgttgggggttatccgaaggagacctttgctgtgtatttgtactgaaccatcattttctttatttttaaagaaaagtagaatgcacacatgtaata

SEQ ID NO: 69 - DNA, 202 bp, Homo sapiens
aggggcaaaggaatttgagtttaggacatccttttaaatgttgtgagtttggacatcctttttaaacgctgtggagcagggtaaaacctgctgtctccgcagacgttgggggttatccgaaggagacctttgctgtgtatttgtactgaaccatcattttctttatttttaaagaaaagtagaatgcacacatgtaatacaa

SEQ ID NO: 70 - DNA, 202 bp, Homo sapiens
gggcaaaggaatttgagtttaggacatccttttaaatgttgtgagtttggacatcctttttaaacgctgtggagcagggtaaaacctgctgtctccgcgaacgttgggggttatccgaaggagacctttgctgtgtatttgtactgaaccatcattttctttatttttaaagaaaagtagaatgcacacatgtaatacaata

SEQ ID NO: 71 - DNA, 202 bp, Homo sapiens
atgaggggattcaattaagaaatctctcttgagcatgaatttctgtatctattaaaaaaggagcaatgaatacttgctaagtatacatggatctaactacagctgttgtttcatgtttcattgttttgtgaaaatggagaaaagaagcagtgtagtggattttttttacacaaatagaaatttgttataaatttaaaaattc

SEQ ID NO: 72 - DNA, 202 bp, Homo sapiens
aattatagatatagtatacataagaatttgtactaagtgccttcattcaaaggtatgccaaatgtgggctatttgtatgcctagcacattcaccttcttcagcagctatgcatgaaaaattcagtctcttatgagggcaccagggcatcctcagttaccatgtgctgactcagcaaatgtccttagcaggtactctggtaaa

SEQ ID NO: 73 - DNA, 202 bp, Homo sapiens
tttcatatagcactggctggttcagagatctcaagtggctgcccaatatttctctaggtaaactctatccatgtaatacaaatttctggccagtatcggaagaaacaaacaaaaatgtttttcttttgttaaatatgtgagtgaagaacaccatactaaaattaattacctccataccttttgtaaaaattcactgagaaag

SEQ ID NO: 74 - DNA, 202 bp, Homo sapiens
cttcaacaatagcaattctttcagtctatgaacatgggatatctttccatttatttgtgtcttcttcaatttctttcatcaatgttttatagttttcagtagtaaagattccttcacttgcttggttacatttttttttgtaaatattttatcctttttgatgctatcatatatgggattggtttcttgatttctttttcag

SEQ ID NO: 75 - DNA, 202 bp, Homo sapiens
ccaccaaatatctggcatactaacatttcaggtgacagaggaagccaaatataagctggttatagaattcaagattctcaagattcttcctcctacctctagtctgtgctgtgaaaagaagaattagcccgtaaatcccagacatactgatacttggatagttagcccatgaagaatgtcatttactcagcctcttgtatga

SEQ ID NO: 76 - DNA, 202 bp, Homo sapiens
aaaagaagaattagcccgtaaatcccagacatactgatacttggatagttagcccatgaagaatgtcatttactcagcctcttgtatgatccttctttgggtgataacttttataatgctatggaaaggctctggctatacttgttacagtcctcacacatccatcaagagatgagaggtagagtgaggggaacatgcgcag

SEQ ID NO: 77 - DNA, 202 bp, Homo sapiens
tggggataacttttataatgctatggaaaggctctggctatacttgttacagtcctcacacatccatcaagagatgagaggtagagtgaggggaacatgcagcagaaaggaggagacatcgctgggaattacagatatttccaacacgatcaattaatcagatggctttttcagataattgctcacaaatctttatactacg

SEQ ID NO: 78 - DNA, 202 bp, Homo sapiens
taattccattataagtattttctttttcttttttttttttttttttttttttttttgagagggagtcatgctctgtcaccaggttggagtgcagtggcgcagatctcggctcactgcaaccgctgcctcccgggttcaagcgattctcctgcctcagcctcccaagtagctgggactacaggtgcatgccaccatgcccagc

SEQ ID NO: 79 - DNA, 202 bp, Homo sapiens
ttctttttcttttttttttttttttttttttttttttgagagggagtcatgctctgtcaccaggttggagtgcagtggcgcgatctcggctcactgcaacctgctgcctcccgggttcaagcgattctcctgcctcagcctcccaagtagctgggactacaggtgcatgccaccatgcccagctaatttttgtatttttatt

SEQ ID NO: 80 - DNA, 202 bp, Homo sapiens
aaatatatttcccaatatgtttttaaattttcatttatgatatatagaaattttaacttttatatattaaaaatatctatatttaaatgactttgtccagagtgtagtgactcatgcctgtaatcacagaattttgagaggctatgtcaagaggattgcttgaggccaggagttcctggccagcctggacgacacagtgaga

SEQ ID NO: 81 - DNA, 202 bp, Homo sapiens
cccataaagttgtcccctgaagaactcaaccttgacagtggattgaatttggtggccctgggatatgggatttctatttctagaaaaacagacactcatcacagtaaactacaggtataccttggagatactgcaggtttggttccaggtcaccacaataaagttaatattgcaataaagtgagtgacacaaattttttggt

SEQ ID NO: 82 - DNA, 202 bp, Homo sapiens
tagagcacatctcctataatattttttgacatgtagttatcaagtttctgacaaagaaatgtatttccttattagtaacaatattgctataaaattatacagttatcaaaccaaactgtcttataaatacaacctggattctgggtttcataaacaaacacagtgcactatgccaaccgaagaataacaggaagtactttat

SEQ ID NO: 83 - DNA, 202 bp, Homo sapiens
aagtactttatgaccccacattatctaacagttctcccctaaacatccctgtagcatgaccaggcaggagatgctacaagactgggaacagagctgcaatctactggcatttttaaacagcttgttgatggactgggcttgtgagaaaaccaacattacactgaaactcttagggatactttaaattggtggattagatggt

SEQ ID NO: 84 - DNA, 202 bp, Homo sapiens
ggagaagatctgtggaacttctcatgcctccattctctctctctatttttttattctttaatctttttagaaggcttactttttacttcctcctgttattctgaatatttatgttttgtgctattctttatttataatttgtcttactatggcaatctgtttgtttgtttgtttccttagtttctcctaggttgtgtgtgct

SEQ ID NO: 85 - DNA, 202 bp, Homo sapiens
attaagcaggaaacctgtctcctgatcagtgtctgaagggttctgtgtcagctctaacagtgttctgctttgggatccaaatagctctgtttgtaggcgcagtctaccttgtagttttcatttttggcaggtttcttttccttatttcattgaaaatgaaattggtttttaaaaagtctcttactgtttcttttcagcatat

SEQ ID NO: 86 - DNA, 202 bp, Homo sapiens
ctttaatatgactgatagagtattttttttagtgtttgaggtttatatatacatatgtttcttttctcttacacatttcatctgagttacttaagactttttctattgatcttagtgttaatatcaattatatctcttaacagacaaagcctttagcaatcttccattattttccccttaatctctctcctattttccatat

SEQ ID NO: 87 - DNA, 202 bp, Homo sapiens
atcctagaccagttaaatcatggtctctgctggaagcccaggcattttttttaaagcttcctaggtgatttcaatgtacagtcaaagttgaaaactgctaagcataacagactgagcagttactgtctccattagcctatcctgtttatgtatcattatttctcagtggaacttttgattaatgctgggttgcaatcaaata

SEQ ID NO: 88 - DNA, 202 bp, Homo sapiens
taccctgatgacaccatgatgttagatcctgctctcatgatccttctatcttagaactgtgttggtttggagcttcagaaattaaacattcttgaataaaagaatgggtctttcactaaataaaattcctgcttattgtgaaaaatatcattgccagttttttgtgttttttttttgtttgtttgtttgtttgttttttttt

SEQ ID NO: 89 - DNA, 202 bp, Homo sapiens
gcttcagaaattaaacattcttgaataaagaatgggtctttcactaaataaaattcctgcttattgtgaaaaatatcattgccagttttttgtgttttttgttttgtttgtttgtttgtttgtttttttttacacagggcagattcttttctccttcaattttttatccctcttttacttaagccaccctttaaagaccctc

SEQ ID NO: 90 - DNA, 202 bp, Homo sapiens
attcttgaataaagaatgggtctttcactaaataaaattcctgcttattgtgaaaaatatcattgccagttttttgtgttttttttttgtttgtttgtttgttttgtttttttttacacagggcagattcttttctccttcaattttttatccctcttttacttaagccaccctttaaagaccctcaattgctgtctgatca

SEQ ID NO: 91 - DNA, 202 bp, Homo sapiens
ataaagaatgggtctttcactaaataaaattcctgcttattgtgaaaaatatcattgccagttttttgtgttttttttttgtttgtttgtttgtttgtttgttttttacacagggcagattcttttctccttcaattttttatccctcttttacttaagccaccctttaaagaccctcaattgctgtctgatcatttaagtt

SEQ ID NO: 92 - DNA, 202 bp, Homo sapiens
tttttttacacagggcagattcttttctccttcaattttttatccctcttttacttaagccaccctttaaagaccctcaattgctgtctgatcatttaagattattacctataactcctggagctttggtctataccaaatcactgtggcctgtggggtctggagcatgaaggaacaaaggtctagggcaattcgtgcctat

SEQ ID NO: 93 - DNA, 202 bp, Homo sapiens
accaatcagaggtactttcaatttcctatctgcccatgaggaacaggtgggagtttgcaaagggtttgcctctggtccttttgctacttaggcatggaaacgttagggttttactttcagtttagtcctaggaagtcagcatgaaacggccttaggttccctgcctccagaccctgttctgcggactcagttgctcacagca

SEQ ID NO: 94 - DNA, 202 bp, Homo sapiens
aagttataggatcatacccaaatagctagatataaagcagaaaacaaagggactgaaggacagtagcaccatggttacaccctgcattctgtatttttaagttctcaccttcataacgtacaaacatttctttgtcccatatttgacttaagttgaatcattacattcaattctagcatgaaatatgtatatacaataagtt

SEQ ID NO: 95 - DNA, 202 bp, Homo sapiens
tttccacccttaatgccattaaagtatgtacctagatgaaccatcattcgtaaagctgaaaggtaatatttacttaatcaaaattagacttcttaatactctgttaaagggaaaacccattgtggggaagaagcatagagtataatgctcatattatatttcagacaataaaacaaccttgtataatcctattatgaacatt

SEQ ID NO: 96 - DNA, 202 bp, Homo sapiens
ccaaatatgtccaaaatttaaagaaagctcctttaaatataactcagttgaaaaggagcttttgattcctggttattggggaaggacctgtttaactattatataccattgctttgttttcttaaaattgatgcaggaggaacatgggtccagcccttttaaaatcctatgttttatcactctccggttgatgggcgcaatc

SEQ ID NO: 97 - DNA, 202 bp, Homo sapiens
gtggtagtgtgggttgcaacccttcaaggatgcttctgagaatcatgttgttaagatagctactgagcaaaaggctgggattctcaaagtcactttgagcagcatgtaataggaggcagagatgaggtcatctccctaaaggtcagctgcatccccagatgtgttcttacctcagccctcacttgaagagagccaaaaaaat

SEQ ID NO: 98 - DNA, 202 bp, Homo sapiens
atgttgttaagatagctactgagcaaaaggctgggattctcaaagtcactttgagcgcatgtaataggaggcagagatgaggtcatctccctaaaggtcacgctgcatccccagatgtgttcttacctcagccctcacttgaagagagccaaaaaaatggctcatccaggggaaagtgtgggacttatgcccacacgtccat

SEQ ID NO: 99 - DNA, 202 bp, Homo sapiens
gcagggcagcccccagcaagcagccaactaagatttttctttctacaacatcccttcactgttctttctccctcatcctctcattacctcctctcaggggagttgtgccagccagggctacccctcacacttttggggccagaacaaaagtaaaataatatcccctctcctgtcccctgcccagtctatggcttggttcttt

SEQ ID NO: 100 - DNA, 202 bp, Homo sapiens
gatctttccctctcatgactcatgctcaaaatttgtccagttcctctctatacgtctcattcataaatgatgttggtcaccaccttctcattcagccactgttcaatacttctgaaaccaattggaatgtaagtaaatacatgcattcctaggtaggtaggtaggtaggtaggtaggtaggtaggtaggtagatagataggt

SEQ ID NO: 101 - DNA, 202 bp, Homo sapiens
tcttcaggaaatcccagtacttagaaaatgaccgtcagccgtgactatcatgccaaacaacttttctccctctatttataccaggtgattgaacaaaaaaacggtaaggggtgtgggggagggcataggctggagctcttgccctggtctgcactattgagtcatcaccataacatgccaaattactcaaataagtctggtc

SEQ ID NO: 102 - DNA, 202 bp, Homo sapiens
atctttctggatggtctgttttttcaaaccctcatgtagaagttaccttctctacccaatagtcaacagaagccaagactcatgaggttcattttatgctagtgaacccatgaaattgtatgacgttaagggttgctctggccctgttcagaagctccaccaatggcaaaatacctttaacaatatgatactgtatcctgaa

SEQ ID NO: 103 - DNA, 202 bp, Homo sapiens
ccaatagtcaacagaagccaagactcatgaggttcattttatgctgtgaacccatgaaattgtatgacgttaagggttgctctggccctgttcagaagctctcaccaatggcaaaatacctttaacaatatgatactgtatcctgaaataaagacctgcaaaatacgatttgacttctgtgctctgtgaaaccatgctcagt

SEQ ID NO: 104 - DNA, 202 bp, Homo sapiens
aatagtcaacagaagccaagactcatgaggttcattttatgctgtgaacccatgaaattgtatgacgttaagggttgctctggccctgttcagaagctccagccaatggcaaaatacctttaacaatatgatactgtatcctgaaataaagacctgcaaaatacgatttgacttctgtgctctgtgaaaccatgctcagtgc

SEQ ID NO: 105 - DNA, 202 bp, Homo sapiens
agtcaacagaagccaagactcatgaggttcattttatgctgtgaacccatgaaattgtatgacgttaagggttgctctggccctgttcagaagctccaccagatggcaaaatacctttaacaatatgatactgtatcctgaaataaagacctgcaaaatacgatttgacttctgtgctctgtgaaaccatgctcagtgcttt

SEQ ID NO: 106 - DNA, 202 bp, Homo sapiens
gacgttaagggttgctctggccctgttcagaagctccaccaatggcaaaatacctttaacaatatgatactgtatcctgaaataaagacctgcaaaatacgtatttgacttctgtgctctgtgaaaccatgctcagtgctttctctctcctagactcgtgcctctcaaacttggatgtgcataggaatcacctggggtcttt

SEQ ID NO: 107 - DNA, 202 bp, Homo sapiens
ggttaccttgtcttccttcgtcatctcaggaagaaataagggcagtgtacacatctccctaagtgtggaaaactatgacgacaccgggtctttctgacgtattgtgtggattcacaaactcccaaagcaccatattatttttaaattagaaatttctgaacagacagaggattcagagctctcacagctaaatgctaaggaa

SEQ ID NO: 108 - DNA, 202 bp, Homo sapiens
attgatttatttagactatacatacttggtttatacgttaccttatttaggattcacagcaatgccgtggggtaggttttcatcatttaaattttacaaaaggagaaaataacatttgtaaaagtctggcgttcaagatggcacaggcggtatgtcaaaacaccagggccaggtctgtctgacatgaaaccttatcagtaag

SEQ ID NO: 109 - DNA, 202 bp, Homo sapiens
aattgagagaaaagtgaaacttggactatggtgaggagctgcaacttgccgcaagttcagatctgttaaggcagtacttctctgtgctttggatctcactctgtgatctttattttagtcacatggaatggtgccttaaaaggctacacccaaatcgaattcccctcaaagtggcctggagtacctcctgaggttaaaaagt

SEQ ID NO: 110 - DNA, 202 bp, Homo sapiens
atctgttaaggcagtacttctctgtgctttggatctcacttgtgatctttattttagtcacatggaatggtgccttaaaaggctacacccaaatcgaattctccctcaaagtggcctggagtacctcctgaggttaaaaagtcacccgaaagagaactgagcacagaggaaataatcactattcctcttttagccttgctga

SEQ ID NO: 111 - DNA, 202 bp, Homo sapiens
actaacatttagtctcaataaccaaaaacaaagaggatcagtattatactcacctcttgaatcaacggtgatctttaattacaatgtctggagtcagagtagtctgccattaaaacatttaatttttagcagtagtaacgagttaagaatgctgatcaaactgcttcaggtagcttcccctttgaggagaggagggtaactg

SEQ ID NO: 112 - DNA, 202 bp, Homo sapiens
gtctggagtcagagtgtctgccattaaaacatttaatttttagcagtagtaacgagttaagaatgctgatcaaactgcttcaggtagcttcccctttgagcgagaggagggtaactgtaaatgaagcagcacattttaaagggcattgctgaaatcttccttgagcagcaccaactggtatttgttgatcactggactccct

SEQ ID NO: 113 - DNA, 202 bp, Homo sapiens
agtaacgagttaagaatgctgatcaaactgcttcaggtagcttcccctttgaggagaggagggtaactgtaaatgaagcagcacattttaaagggcattgcttgaaatcttccttgagcagcaccaactggtatttgttgatcactggactccctttagctgctgtttctgagaacaacctgcctcctgccttaccccattg

SEQ ID NO: 114 - DNA, 202 bp, Homo sapiens
taggtctagaaagagctgggtctagaaagactctggctagtgctgctccatcacaccttgagaattgttgccgatattgatgtttgagagggcttacctgcgttaggcacctgcgcaccatgtgatctcttctcttctcgtgctatggtgagcctagaatttgaggaaacctggaaagttgcatccagggaagactgaagtc

SEQ ID NO: 115 - DNA, 202 bp, Homo sapiens
gccttgagaactatgtgtgtagaaggcagacatcagaaacatgctgcttgcgggacaggagagtaggaacttgcatatagtgaatgtctgccatatctatattcctttagcaaaccatgtaaagtagagctatctccaatttatgagttaaaaacttgaacaaccttaccatgattaggcaaacttgctcaaggttaacaag

SEQ ID NO: 116 - DNA, 202 bp, Homo sapiens
tgtagaaggcagacatcagaaacatgctgcttgcgggacaggagagtaggaacttgcatatagtgaatgtctgccatatctatttcctttagcaaaccatagtaaagtagagctatctccaatttatgagttaaaaacttgaacaaccttaccatgattaggcaaacttgctcaaggttaacaagtcattgataaactcagt

SEQ ID NO: 117 - DNA, 202 bp, Homo sapiens
tagaaggcagacatcagaaacatgctgcttgcgggacaggagagtaggaacttgcatatagtgaatgtctgccatatctatttcctttagcaaaccatgtacaagtagagctatctccaatttatgagttaaaaacttgaacaaccttaccatgattaggcaaacttgctcaaggttaacaagtcattgataaactcagtat

SEQ ID NO: 118 - DNA, 202 bp, Homo sapiens
aagcccatctgccactttacccagtgatctttgatgcctttcctagtactgtctaactctctttaaaaatacagaaacagttactgtggaattctatgcgagtcctccatggcctccttcttccaataactccgaatggaaattcctgtgggggaatagcccaataaacacaattatccagcagaagaagggctttctggtt

SEQ ID NO: 119 - DNA, 202 bp, Homo sapiens
ctgccactttacccagtgatctttgatgcctttcctagtactgtctaactctctttaaaaatacagaaacagttactgtggaattctatgcgatcctccactggcctccttcttccaataactccgaatggaaattcctgtgggggaatagcccaataaacacaattatccagcagaagaagggctttctggttggatcagt

SEQ ID NO: 120 - DNA, 202 bp, Homo sapiens
gtggaattcaccatgtagttgcatacagcaatattttatttattcttttgctgaatattattccattacttttgccagtttgaatggatttctttcttacaggtcaagtaggccatgactaagggagataatgaagcatggaattgtcttttccagagaaaccacaggcatggaataagaattccctgaggaggccttttct

SEQ ID NO: 121 - DNA, 202 bp, Homo sapiens
gtcctccccaccccacaacacatggggattatagggctacaattcaagatgagatttgggtgaggatacagccaaaccatatcacctaccttactcattaagtctcttggttcaatactgaatccccagtaccttgaatattagcacgtacaggtcactcaaaactgtgtgttcggtgaatgaataaattgcacaaaaattc

SEQ ID NO: 122 - DNA, 202 bp, Homo sapiens
gagagactaaaaaggagcatttgcagtagtgagaaaaccaggcaaagaggagttgaggagttaggaaactaaaccagcgtactaaacagaaggagtggtcctgaagtgtcaaacgctgttgattgtggcagaataaacagcagtggtgaaatgaagacaggaggtaggttataggtgagagaggagtgaagtgctaatgtct

SEQ ID NO: 123 - DNA, 202 bp, Homo sapiens
gaaaaccaggcaaagaggagttgaggagttaggaaactaaaccagcgtactaaacagaaggagtggtccgaagtgtcaaacgctgttgattgtggcagaaataaacagcagtggtgaaatgaagacaggaggtaggttataggtgagagaggagtgaagtgctaatgtctctttctgtaatagatgctctacctagaaaaaa

SEQ ID NO: 124 - DNA, 202 bp, Homo sapiens
ttttttcctaccttatctcgtttccagatatctgcctaatttctcctagttctggtgctacatcctcctctaacttagaatcacgcttaagtgctataggctcaagtatgttgtgtgttagggaggaacagagtactttctatttgcatcaaaagggattttcatgtttaatggcaggaaggattttggtttgtgggtgagg

SEQ ID NO: 125 - DNA, 202 bp, Homo sapiens
tcctctaacttagaatcacgcttaagtgctataggccaagtatgttgtgtgttagggaggaacagagtactttctatttgcatcaaaagggattttcatgatttaatggcaggaaggattttggtttgtgggtgaggagagggggtgctgagagggaagtctgtcttgattctaaaaacggagaatatttctaaagtaaaat

SEQ ID NO: 126 - DNA, 202 bp, Homo sapiens
tgcatcaaaagggattttcatgtttaatggcaggaaggattttggtttgtgggtgaggagagggggtgctgagagggaagtctgtcttgattctaaaaacaggagaatatttctaaagtaaaatatcccttctcatgtatgccagggtcctgggcaatgacagctccaaaatatcagccactttcccctttttgactggggc

SEQ ID NO: 127 - DNA, 202 bp, Homo sapiens
aatctaaacaatttctcctagaaggtaatgaggttttacacaaaccttattaagtcagtcctttgctaattgaatgtttaaaatctggggcctactgggtagtaacatttattgagacaaagtaattaacattttctttcttgatgcatttctttcaatattatgcttaaaacctcaacacatagtagaatgcttagcatat

SEQ ID NO: 128 - DNA, 202 bp, Homo sapiens
cttccaaatacttggtctaggaggaaaataaggggaaaacatagtgaggatatattgaaaaatattttttttctcagaagaaaggagatacagtttacatataatttgagaattcggcattgcttaattctccctaatgtctacgagaacttctgctttaaaaccagccccaagaacacattggtcagccagctcttgtgct

SEQ ID NO: 129 - DNA, 202 bp, Homo sapiens
catacagtagtccccttatccacagttttgctttctgtggtttcacttacccagtcaaccaagagctgaaagtaggtgagtacagtacaataaaattcttctgagggattacagtctcatgacttttgtggtatattgttataattgttctattttattattggttattgttgttaatctcttactgtgcctaattcataaa

SEQ ID NO: 130 - DNA, 202 bp, Homo sapiens
aataaaattctttgagggattacagtctcatgacttttgtggtatattgttataattgttctattttattattggttattgttgttaatctcttactgtgctctaattcataaaccaaactttatcataggtataacgtgtatagaaggaaacggtgtatatagggttcagtaccatccacggtttcaggcgtccatagggg

SEQ ID NO: 131 - DNA, 202 bp, Homo sapiens
tccccaggaatgcttcctgggtttaccttacaaatagacttcccaaacttgcaatattatatctcaagttctgcttctgggagaaccccattcaagacagacgtcacatggtccttataacggtctacacagagattattagggcctcatctaaaacagtgaccatgaaaaaagtggaaggcaaaattctaagatattgccc

SEQ ID NO: 132 - DNA, 202 bp, Homo sapiens
gtttaccttacaaatagacttcccaaacttgcaatattatatctcaagttctgcttctgggagaaccccattcaagacagagtcacatggtccttataacaggtctacacagagattattagggcctcatctaaaacagtgaccatgaaaaaagtggaaggcaaaattctaagatattgccccaaatccacatcccctgatg

SEQ ID NO: 133 - DNA, 202 bp, Homo sapiens
taaaaatacaatccaaagctcaggaacaattctgggtctgaagaacaaacatttggaagtcattggcatatgtttaatgttgaaatcatggaggatcgccagtacaatgtggcacatatacaccatggaatactatacagccataaaaaaggatgagttcatgtcttttgcagggacatggatgaagctggaaaccattatt

SEQ ID NO: 134 - DNA, 202 bp, Homo sapiens
agccataaaaaaggatgagttcatgtcttttgcagggacatggatgaagctggaaaccattattctcagcaaactaacacaagaacagaagccaaatatcagtgcgttctcactcataagtgggagttgaacaatgagaacacatggacacagggaggggaacatcacacactggggcctgttggggggtggggggctagag

SEQ ID NO: 135 - DNA, 202 bp, Homo sapiens
attgtattccatctaatttacaataaagtaatttttaatgttctggcttgtgaagtagggtatggatagcaaatacatggcactaaagccaccattccccagattctgagcctatggcattactaatcaattcagtaattttcccttcagaaccagtattctttccaaccatgctctgggcagcaacgaattgattatagtt

SEQ ID NO: 136 - DNA, 202 bp, Homo sapiens
ttttcccttcagaaccagtattctttccaaccatgctctgggcagcaacgaattgattatagttcacactcatttgcaatcatgggttagtttcaaaaccagtatccatttaataacagatacgttgagttgctacttgagggtggagggtgagaggaggatgaggatcaaaaatgattgcttattgtctgtgtaatggaat

SEQ ID NO: 137 - DNA, 202 bp, Homo sapiens
cccttcagaaccagtattctttccaaccatgctctgggcagcaacgaattgattatagttcacactcatttgcaatcatgggttagtttcaaaaccatatctcatttaataacagatacgttgagttgctacttgagggtggagggtgagaggaggatgaggatcaaaaatgattgcttattgtctgtgtaatggaataaac

SEQ ID NO: 138 - DNA, 202 bp, Homo sapiens
tattgttgaatgatattagatagcctcctgaggaatttgttaagatggcaagaagtggttcctacaggtggtttggatatgaaacattgcaaagctaagtctgaaatagcatgaaaaaagtataaataggaatttcctttgatccttctaataaggctgaatggtgttgtgtccacagttattaagttcaaatattataatt

SEQ ID NO: 139 - DNA, 202 bp, Homo sapiens
cctaagaaaaaaaaagcttaggcagctccatttctggatcacagggtgttaacatattagtcaacccataaaagctcccactgatttatatggagttgtaagcgttgcgtcacaggtggaggggcctaaattgtttttggagaaatatagggctctgtcttcaggatcatgacccgtgagtcacacccagggctcccaaagc

SEQ ID NO: 140 - DNA, 202 bp, Homo sapiens
gttgcgtcacaggtggaggggcctaaattgtttttggagaaatatagggctctgtcttcaggatcatgacccgtgagtcacacccagggctcccaaagccagaggccagtggggacgtggaagaacttggcttagccacagggatgtgactcactaaaaaagatgaactggttttgaaaagtaacaagaaccaccttctatt

SEQ ID NO: 141 - DNA, 202 bp, Homo sapiens
acagtcctgccactgctctccagcccaggcaacagagtgagacactatctcaaaaaaattaagttgaaaacaattttaacattttaatatgtgagcatccactttctatgactgctgaattttattctaagaatagataaaaagttaatttcaagtatcgaaaacttgccactttcttttttcatgtttggattacaggcct

SEQ ID NO: 142 - DNA, 202 bp, Homo sapiens
aaatgagaatgaatggagatgtatctgacgttttgggagcaggctagattcagatcttctctccctgtagctaaattgggtgggcagctggcctcaagaacgagtccacccacaacctccaactcctggcacgaacctgcttgagcttggaagacattgtcggccctagatcccaccctgactcctgcatgttctggacacc

SEQ ID NO: 143 - DNA, 202 bp, Homo sapiens
agcaagaagaatattggcagaaacttctattttcaatgcagtttttaggaaggttaattactttgtgtttctgaagatgtaaatgttcctcaaggaagaaactctctgaataaaactcaatgacaagatctttataatggacttctggttattgctatgtgcaatgccctacctataactcgcattttaattgatgtatcct

SEQ ID NO: 144 - DNA, 202 bp, Homo sapiens
gctatgtgcaatgccctacctataactcgcattttaattgatgtatcctgagcaagatttgaaacagatctctttccctctcttctaattttaatgagtccttctctagcaagcacctatatcaccagagagatgcagagaacctcagcatactctggatgggcctccctctgcaatttatgatgaagtctgacttcagagt

SEQ ID NO: 145 - DNA, 202 bp, Homo sapiens
ttaattgatgtatcctgagcaagatttgaaacagatctctttccctctcttctaattttaatgagtcttctctagcaagcacctatatcaccagagagatgtcagagaacctcagcatactctggatgggcctccctctgcaatttatgatgaagtctgacttcagagtgagttcgcagcctccttcctgcctctcctctgg

SEQ ID NO: 146 - DNA, 202 bp, Homo sapiens
tgtatcctgagcaagatttgaaacagatctctttccctctcttctaattttaatgagtcttctctagcaagcacctatatcaccagagagatgcagagaaacctcagcatactctggatgggcctccctctgcaatttatgatgaagtctgacttcagagtgagttcgcagcctccttcctgcctctcctctggtttccagg

SEQ ID NO: 147 - DNA, 202 bp, Homo sapiens
tatgatgaagtctgacttcagagtgagttcgcagcctccttcctgcctctcctctggtttccagggtagcagactcttgtctcccttcagacctgggctcctcccagctcagtcccacttggcatttctaactgaggttatggatacacattgagcccagaactcattgcttttttggaaaaggcaaaataagagaggggat

SEQ ID NO: 148 - DNA, 202 bp, Homo sapiens
taaacctattttgtaagtgaattaggtcttaggagagaatcagaaacgatgttgttcttttagccgcttacagtgtttgctttaaacccttgataggaacagtgggcttgtgacagcagcggcagaggtaaacagatgactttatatataatgctttttacaaggtggttcagttcgtttctaaccttggtctcggcgtcat

SEQ ID NO: 149 - DNA, 202 bp, Homo sapiens
cgtcctcccttcaagggcaagaaaatgtccattttgcctttggtttcacagctctttctccacagtagagcttgcataatgctataccccccaatcccagaggaaccccggtagtgtcaccaggtatttttattattatcattattattttcatcaggtgactgtgaatgttcacaataaaagttgcaatttttatgagcca

SEQ ID NO: 150 - DNA, 202 bp, Homo sapiens
ttgcagaagacatggggaatagtagagcaaggtctgcaagattctggcctgaaggaaaaggctccatagctctgcttttgagccatggaagctgcctgccctctcccttttcctggaaggtcccatgactagtttcctaataccaactagtcaagaatatgtagggctaaagggacattaatattaacataagtgatacagt

SEQ ID NO: 151 - DNA, 202 bp, Homo sapiens
ggaaagccagctccttgctggggaccttgggctagtgcttagtatctgatttcttcatctggaaaatagctataataatggaacccatcttagagggttggtttaacattatatgaaaaaatgaaagttcaatgcacagtctgtgatctgatataggtcaggatgcaatattgttaatactattagctactataaatattat

SEQ ID NO: 152 - DNA, 202 bp, Homo sapiens
cagctccttgctggggaccttgggctagtgcttagtatctgatttcttcatctggaaaatagctataataatggaacccatcttagagggttggttaacacttatatgaaaaaatgaaagttcaatgcacagtctgtgatctgatataggtcaggatgcaatattgttaatactattagctactataaatattattggtaag

SEQ ID NO: 153 - DNA, 202 bp, Homo sapiens
acccctgcacccatgatccaggtcgcatcaggcctgtttgtgcactctcagagtttaacggaagaacccatctttcatacttcattatttaaagaaggctaggactgattttaaaagcacaatgtacatgaaaatcacctgaagtatttgataaaatccatatttctataccaacaccctcaatttgagttgatgggtctga

SEQ ID NO: 154 - DNA, 202 bp, Homo sapiens
agttgatgggtctgaggagaggccctggtttctatattttaaagaactccctaaatgcttctgaggaaggtgctgcttgaacaagaaggtgcccttctgcctaagagacaagccttttccagcttgaaaactattattttaccattctatattctgagcaggaaatgaatggacagactgtatgtagcgatgtgatatacaa

SEQ ID NO: 155 - DNA, 202 bp, Homo sapiens
catgtgcttgctacattctagggcaggggttgaaatctcaaacacacagaggagccaggcaagatggtgctgatataaaagattcagggttctctcggtccttctatttttgcaatttgaggtaaacagtttggctgcataagaaaaacaacaggctgaatgtaagagttcatgctgtcaaactttggttcacagcatccgg

SEQ ID NO: 156 - DNA, 202 bp, Homo sapiens
gctcaaggtagtgggactcattccactgtggctcagagcatcaagcacaagtgttcctgcaaagaaggagggagctgcatggccttttatgatccagtctctagaagtcatacaacatcacttcagtcatactatattggtcaaaaccgttccaagcccaaccagattcaaagacaggaaagatagtctctacttcttgatg

SEQ ID NO: 157 - DNA, 202 bp, Homo sapiens
ccaaccagattcaaagacaggaaagatagtctctacttcttgatggggaagtgacaagtttacattctagaagagcgtgagagcgagagatattattgcaaggcatctttgaaaaatagaatttgctgtcataactaattcaaaactttgttttatcctgtaatcccagaacttcaggaggctgaggcgggtgtatcacttg

SEQ ID NO: 158 - DNA, 202 bp, Homo sapiens
gaggcgggtgtatcacttgatgtcaggagttcatgacaagcctggccaacatgatgaaaccctgtctgtactaaaaatacaaaggttagccaggcgtgataggtgcgcgcctgtaatcccagctactaggaaggctgaggcaggagaatcgcttgaacccaggaggtggaggttgcagtgagcccagattgtgccactgcac

SEQ ID NO: 159 - DNA, 202 bp, Homo sapiens
agagttggatgaaagtagggagggtactgagactaggaggcagctatttattagaataacctggaaaatggaccaatcccccccattcccagccccaagaatgtttacctctatccagaagaggagcatttgcatttttaatattttgagaaggttcagaaacccgagcttcctactgcttcctattgagaagcactgttct

SEQ ID NO: 160 - DNA, 202 bp, Homo sapiens
gttctaaacagacttctacattgcaattggtactcacatatttagacatcagtcttaccgcttaaattcttcatttgcttgtaaaagccatgtagctctaacactcagaataatccattgtctactgcccttcatccctcatgatcaagtttttctaaatatgtttggaaattgacatgtgatttaaaaataacttaaagta

SEQ ID NO: 161 - DNA, 202 bp, Homo sapiens
tctaaatatgtttggaaattgacatgtgatttaaaaataacttaaagtatgtctgatagcccagataaggacatgtactttgtagtgccattcatggaaactagtgaccttgaaaaaaaattctggagggccttcctctcatcttcctttctccagttcctatggatatggaaggatagggctaagaattgctgtctgcctt

SEQ ID NO: 162 - DNA, 202 bp, Homo sapiens
taataaggagacttttgctcaaataaacactgagttcttttttttttttttttccaagccattcaccaactcatataatgtatctttgtgaggtctgttcacttccataggataattcagaaagttatacctaggctgggcacagaggctcacactgatgatcccagcaccttcagtggctgaggtggttaggattgcttga

SEQ ID NO: 163 - DNA, 202 bp, Homo sapiens
gggcacagaggctcacactgatgatcccagcaccttcagtggctgaggtggttaggattgcttgaggccaggagttcaagaccagcgtgggctacacaacagagcctccatctctatgaaaaaatttgaaaacataattttttaaaaggaaagttatacctgggccagggattctgtaccgttcatgatcttgtattccagg

SEQ ID NO: 164 - DNA, 202 bp, Homo sapiens
gaccagcgtgggctacacaacgagcctccatctctatgaaaaaatttgaaaacataattttttaaaaggaaagttatacctgggccagggattctgtacccgttcatgatcttgtattccagggtgtgtaatattaggaaggtttaggtttattgtctaaatgttaattgtgtaaataaggtggtaaccatggcaacccaat

SEQ ID NO: 165 - DNA, 202 bp, Homo sapiens
tgtaccgttcatgatcttgtattccagggtgtgtaatattaggaaggtttaggtttattgtctaaatgttaattgtgtaaataaggtggtaaccatggcaagcccaatcccacccccctcagcattggcaggtggccagggaggtgtgatcacaacttctatctgagctcacctttgcaggtaatggggcaggtatgtggca

SEQ ID NO: 166 - DNA, 202 bp, Homo sapiens
cagggtgtgtaatattaggaaggtttaggtttattgtctaaatgttaattgtgtaaataaggtggtaaccatggcaacccaatcccacccccctcagcatctggcaggtggccagggaggtgtgatcacaacttctatctgagctcacctttgcaggtaatggggcaggtatgtggcattaaagagcaggaaggccagcaag

SEQ ID NO: 167 - DNA, 202 bp, Homo sapiens
agggtgtgtaatattaggaaggtttaggtttattgtctaaatgttaattgtgtaaataaggtggtaaccatggcaacccaatcccacccccctcagcattaggcaggtggccagggaggtgtgatcacaacttctatctgagctcacctttgcaggtaatggggcaggtatgtggcattaaagagcaggaaggccagcaagg

SEQ ID NO: 168 - DNA, 202 bp, Homo sapiens
ttaactcttccagtggcactcctctgagtacatggaccacacctgcaaacctggcaggacgtgccgtgcatgactggacggaggcactgatacactcggaagccaaggaatgggtttaggcaggagctcagggctgaaatagggagcccagcctgccgggctgcttgaaatctcaggactcataatgcttgtgctttaggga

SEQ ID NO: 169 - DNA, 202 bp, Homo sapiens
aggacgtgccgtgcatgactggacggaggcactgatacactcggagccaaggaatgggtttaggcaggagctcagggctgaaatagggagcccagcctgcctgggctgcttgaaatctcaggactcataatgcttgtgctttagggagacaactgctcattagctggatgaaattaaccagtcatgtcaccttttgacacct

SEQ ID NO: 170 - DNA, 202 bp, Homo sapiens
aaatctcaggactcataatgcttgtgctttagggagacaactgctcattagctggatgaaattaaccagtcatgtcaccttttgacacctcaatttcctcagttactgaatagtgagggatttgaacatattatccctaagctaccttttagctctaacatagactgagtatagtctatgtcaagactctgactagagtgta

SEQ ID NO: 171 - DNA, 202 bp, Homo sapiens
agaactcaacaaaacacaaaccactgagctccacccccacccagattactgattctaaagatctgaggtggggcctgagaatctgcatttctaacaagtactccaaatgttgctaagctgctgatttggggcccatagtttgagaaccactgttctggtctgttattgcttggttatgttctggggattttcctgggccacc

SEQ ID NO: 172 - DNA, 202 bp, Homo sapiens
gaaatatattcagcacagacagtgtgatatatacttgggattcatttttatctatactctaggagctcattgtatgctgcttctaaaatagctcttaatgactttccacctcctagtattcacacccttgtgtaatccactccctctccatgtgggttaaccctggtgacttgtttccaaactaatagaatgtgacaaaggt

SEQ ID NO: 173 - DNA, 202 bp, Homo sapiens
tggaagtcgatccctccccagtcaagctttccaatgagaccacagaaaagaccaaccccttgatcacagtcttcaaggagaccctgaggcagaggacctaagttaagtcctgtcaagatttctgaagcacaaacactgagtcaatgaatgtgtgttgttacaagctgccaggttaaagataatttttaatgcaacgtgagat

SEQ ID NO: 174 - DNA, 202 bp, Homo sapiens
gcactgtactttaaaaaccctactctaagccctagctctcactgtatctttggtgaataacagaatcctggtatatttgcataatggtgtgcagagagtggttgttgcattcatgaagccagggttgttaaaatcatgtggattgtgggataatattacacagggctgctcccttgtgtgaacagggtgggtgaccccaaga

SEQ ID NO: 175 - DNA, 202 bp, Homo sapiens
ctctcactgtatctttggtgaataacagaatcctggtatatttgcataatggtgtgcagagagtggtgttgcattcatgaagccagggttgttaaaatcactgtggattgtgggataatattacacagggctgctcccttgtgtgaacagggtgggtgaccccaagagtctgcaaacttctgaatagcatctttagcctagg

SEQ ID NO: 176 - DNA, 202 bp, Homo sapiens
aacttctgaatagcatctttagcctaggaatatgaaataatcccaactctgtttatagcaggtcatcaatacctgaattgggtgaaattttggcatggaaagtatatatatttttaatctgtctcaagtgtaccttctgggaaaggctctgtggaagcatctagctagacagagaattaagtaaggaattcctggcacttgg

SEQ ID NO: 177 - DNA, 202 bp, Homo sapiens
cttcacaggctcaagtttcacagtcttccctgacaggaaatcctagctaaccttgaacttcagggtcatctgttgtggtttaacaccattttttcatttcctagttaaaagggcagctctgctcactgtcccttgcatgtcatgccctgaaaacatctcaactccctcttgttactgcctgaaaaatggattccccgaaata

SEQ ID NO: 178 - DNA, 202 bp, Homo sapiens
agtttcacagtcttccctgacaggaaatcctagctaaccttgaacttcagggtcatctgttgtggtttaacaccattttttcatttctagttaaaagggcacgctctgctcactgtcccttgcatgtcatgccctgaaaacatctcaactccctcttgttactgcctgaaaaatggattccccgaaataataagtcctccaa

SEQ ID NO: 179 - DNA, 202 bp, Homo sapiens
atttctagttaaaagggcagctctgctcactgtcccttgcatgtcatgccctgaaaacatctcaactccctcttgttactgcctgaaaaatggattccccagaaataataagtcctccaaattcagacatcagctttcctcagttaatttctgcatcgtcatttataatattggaaggttcttacttttaagaaaatttgga

SEQ ID NO: 180 - DNA, 202 bp, Homo sapiens
tctcaactccctcttgttactgcctgaaaaatggattccccgaaataataagtcctccaaattcagacatcagctttcctcagttaatttctgcatcgtcagtttataatattggaaggttcttacttttaagaaaatttggaccaatgccccaaatggtcttcccatctcagagcaaccaaaaaatgtgactgtggctgct

SEQ ID NO: 181 - DNA, 202 bp, Homo sapiens
tgccaggacccctacatgcaaatgaccagactgtagagagttaagtcttgggacccacacatttctcccaggggaagtcattatttcagggcttttcactgttgttcatgaatttcttctgtgatctaggccttggggagaaagaacattcacctaatttcctataaaaggcactgtagcatagaagtaaagacaatcagtg

SEQ ID NO: 182 - DNA, 205 bp, Homo sapiens
gaagtcattatttcagggcttttcactgtgttcatgaatttcttctgtgatctaggccttggggagaaagaacattcacctaatttcctataaaaggcactgtagtgtagcatagaagtaaagacaatcagtgttcacatcatagttttgtcttttatggcccgtataatcttgggaaaattacttaacgtctccgtgctaatgt

SEQ ID NO: 183 - DNA, 202 bp, Homo sapiens
tgtagccaaccagtttcttagttcattcagcttgctttttgtactccaggagtgtatcagttagctcttgctatgtaacaaatttctccaaatttagcagcgttacaacaacaaacatttattatctccagtttttgcaggtcagaaatttctgagtagcttagctgggaggttctggctatgggtctttcatgaggttgca

SEQ ID NO: 184 - DNA, 202 bp, Homo sapiens
aactaactggccccagggactggattcagctctcttactgttgttatgtgcagctgtacctcattttcaagtcatgtcgatggagtcacttcactgatatagctgtggcacatcctgaagccctaggctactgagagattgaaacttgtatatgatatctttagacttgcactgtctagtatggtagtcaatagctacatgt

SEQ ID NO: 185 - DNA, 202 bp, Homo sapiens
tctctgctgactctgcagagtgggtgcctgtgattttacaggtctgctccttcagacctttctgaacaacactaataactaagggtgtcctgtacaactgacaacaatggtttccaatggtgtgtgctctggtgagctcaaaacattccctgaggactgaacttccactcaaaagctgggtcccaacactctacttcctctt

SEQ ID NO: 186 - DNA, 202 bp, Homo sapiens
cgttttcagtttctttctcactgacccatgccattcaatgtcttcatcctcctcaaaaccttttttggaataaggcaggaaataaataaataaataaatgacaactgtctctaatgtaaaatgagttgggtagtttgaagatgtttcctctgaaacacttgaaggaagataaagactgaaaagagagaaactgctatatttt

SEQ ID NO: 187 - DNA, 202 bp, Homo sapiens
caaaagggtaagagaagtaagaaagctggtttagatcagaaaagacatctcctccccccagaattttctggaagatgacattttgtaacaataagtttatattccagtttgtaaatcttttggttacataaaacaggaccatgtctatgtcaagtaatggaggttactgttgagttactatgacgatggcacaggcagaagc

SEQ ID NO: 188 - DNA, 202 bp, Homo sapiens
ccccccagaattttctggaagatgacattttgtaacaataagtttatatccagtttgtaaatcttttggttacataaaacaggaccatgtctatgtcaagctaatggaggttactgttgagttactatgacgatggcacaggcagaagctagtgcctcccttagactagggcagtagttctcaacagtgggtgggagcaggt

SEQ ID NO: 189 - DNA, 202 bp, Homo sapiens
cgatagtgctgaggatgaaaaaccctgaacaaggacaagaggtgaccccctactccctgtgtggccctgtgacctggaggactttgtaaactaaacatcaagaaaacaaggacaataaatattgtttttggttttcacattcataataaaattctttacttgattgagtataaagttttaagacaagagaagctgacaagca

SEQ ID NO: 190 - DNA, 202 bp, Homo sapiens
aataaatattgtttttggttttcacattcataataaaattctttacttgattgagtataaagttttaagacaagagaagctgacaagcaatagacaaccactgttgcccatcataaaacctttatttatattcagagaacagacattgagtttcagttaagtttcccttggaaataatcatgcaaaggctgtatcattaagc

SEQ ID NO: 191 - DNA, 202 bp, Homo sapiens
tttttggttttcacattcataataaaattctttacttgattgagtataaagttttaagacaagagaagctgacaagcaatagacaaccacgttgcccatcagtaaaacctttatttatattcagagaacagacattgagtttcagttaagtttcccttggaaataatcatgcaaaggctgtatcattaagcattgtaacagg

SEQ ID NO: 192 - DNA, 202 bp, Homo sapiens
atattcagagaacagacattgagtttcagttaagtttcccttggaaataatcatgcaaaggctgtatcattaagcattgtaacaggtagatgaggataaggtcttcctcatccctaaaattcttctgaagtagaatagacacccacctgcctggagtggttaaggtaagaaggaaggacaggtaatcctggagagaggtact

SEQ ID NO: 193 - DNA, 202 bp, Homo sapiens
aggtctagattctatctttctaaatattacagaatgactcaatagcacattctactaaaaaatggtaatgtatcatttaaaaactcttcaaaaaatggttattgaaactgcacttactgttgcttattatactatcttgatcataattttgtaatgtttgagagaggtactctctcctcacctccccctcacctctcagatc

SEQ ID NO: 194 - DNA, 202 bp, Homo sapiens
gcacctcttgcctggtagtggctaccggtgtccatgattacagcttctgcttggtggaaacttctttgcggttccagttttttctggctctgtcaaccctagtttttatttttttgtctttcaggtcaattggaaaaggcctctagctgttgctggactcagagttcctcaacatcttttatcagttccattaaaactatgg

SEQ ID NO: 195 - DNA, 202 bp, Homo sapiens
tctagctgttgctggactcagagttcctcaacatcttttatcagttccattaaaactatggggagggataacattaggagaaatacctaatgtaggtgacgtggttgatgggtgcagcaaaccaccagggcacgtgtatatcttgtcacaaaactgcacgttctgcacacataacccagaagttaaggtttaataaataaaa

SEQ ID NO: 196 - DNA, 202 bp, Homo sapiens
tatacaatctagcaattgtgctccttggtattaactcaaatgagataaaatcttatatctgcacaaaaactccacccagatatttatagcagctttattcagtaattaccccaaactggaagcaaccaagatgtccttcagtagatgaatggataaacaagctgtggtcattcaacaatgggatattagtcagagataaaaa

SEQ ID NO: 197 - DNA, 202 bp, Homo sapiens
ctttattcataattaccccaaactggaagcaaccaagatgtccttcagtagatgaatggataaacaagctgtggtcattcaacaatgggatattagtcagaggataaaaataaatgagctgtcaagccatgagaagacatggaaggaccttaaatgcatagtgataagtgaaagaagccagtccagaaaggctgaatactct

SEQ ID NO: 198 - DNA, 202 bp, Homo sapiens
ttctgtatgatactgcaatggtggatacaaggcactctgcatccttccaaacccatagaatgtacaacacaaaggctgaaccctaatggaaaccctggacatttcattaataataatgtattgatactagttcatcaatcgtaagaaaggtaccacaccaaagcaagatattaataataggggcaactgtgaaaggcatggg

SEQ ID NO: 199 - DNA, 202 bp, Homo sapiens
tgtattgatactagttcatcaatcgtaagaaaggtaccacaccaaagcaagatattaataataggggcaactgtgaaaggcatgggaggcatatgggaacctctatactttcagcttaacatctctatagacctagaactactttaaaaatgaagttcattaattaaatatatcagtgaaccatgtatatatatgaggaaaa

SEQ ID NO: 200 - DNA, 202 bp, Homo sapiens
atgttagacaccaaaatattggtggcttttcatatcaccaatgctaaaaaattaattaaggggtatatatcttttttattccttggcattacctaagattctagagaataaagaatgtccaaacaccctggttttcctggggcaatcctgatttagggctgttttcctgaatgcagtcgttaacggaaccccgttttcctct

SEQ ID NO: 201 - DNA, 202 bp, Homo sapiens
aagaatgtccaaacaccctggttttcctggggcaatcctgatttagggctgttttcctgaatgcagtcgttaacggaaccccgttttcctctcaaacttgctcctagtttggataataagttatattattattgtgcctctaatactttctttgcaaaaggagttacaaagcaccatgggcagagtctagtcatactcaccc

SEQ ID NO: 202 - DNA, 202 bp, Homo sapiens
ccgttttcctctcaaacttgtcctagtttggataataagttatattattattgtgcctctaatactttctttgcaaaaggagttacaaagcaccatgggcaggagtctagtcatactcaccccagcttgtctgtttgggcactgtctggaacagatagactaggctgggaacgaaggtgtgtctgccaggaccaaacgttcc

SEQ ID NO: 203 - DNA, 202 bp, Homo sapiens
ttgcaaaaggagttacaaagcaccatgggcagagtctagtcatactcaccccagcttgtctgtttgggcactgtctggaacagatagactaggctgggaactgaaggtgtgtctgccaggaccaaacgttccaaaaacaaattttttatcagatgaaactataacctagatcttataatgattaaatgcaatttctgtagtg

SEQ ID NO: 204 - DNA, 202 bp, Homo sapiens
aattttttatcagatgaaactataacctagatcttataatgattaaatgcaatttctgtagtgaaaatcaccttaagtagtataaggatttgctgaagatagaacatcaggccccatgagttcctcaacagctcgaaccatggagagttcgtgcaggcagggtcatttgtctaccacctacactgtggatcaggtacagagt

SEQ ID NO: 205 - DNA, 202 bp, Homo sapiens
ggccccatgagttcctcaacagctcgaaccatggagagttcgtgcaggcagggtcatttgtctaccacctacactgtggatcaggtacagagtacagaggcgcttgctctatacatatatagctactaaccagctccagaagccaatattttacctaccaggaccaggaccttgaggtttccctttattattatccattcat

SEQ ID NO: 206 - DNA, 202 bp, Homo sapiens
tcgaaccatggagagttcgtgcaggcagggtcatttgtctaccacctacactgtggatcaggtacagagtacagagggcttgctctatacatatatagctagctaaccagctccagaagccaatattttacctaccaggaccaggaccttgaggtttccctttattattatccattcattcaataaatacccattcaattca

SEQ ID NO: 207 - DNA, 202 bp, Homo sapiens
cattgaaaatacagtggtgcataacacaagtaaaaaatatccattctagtgggaagaggcaaccaataagcaagtaatcaagtcagtgaaacaattcatactaatggtgaatactaggaagggtatagatgcaaaatcattgcacaggagagagggagggcttctttctagagatgatagttgaactaattctgaagaggaa

SEQ ID NO: 208 - DNA, 202 bp, Homo sapiens
aaaatacagtggtgcataacacaagtaaaaaatatccattctagtgggaagaggcaaccaataagcaagtaatcaagtcagtgaaacaattcatataatgagtgaatactaggaagggtatagatgcaaaatcattgcacaggagagagggagggcttctttctagagatgatagttgaactaattctgaagaggaaaggga

SEQ ID NO: 209 - DNA, 202 bp, Homo sapiens
ggtgcataacacaagtaaaaaatatccattctagtgggaagaggcaaccaataagcaagtaatcaagtcagtgaaacaattcatataatggtgaatactagtgaagggtatagatgcaaaatcattgcacaggagagagggagggcttctttctagagatgatagttgaactaattctgaagaggaaagggagccagccaag

SEQ ID NO: 210 - DNA, 202 bp, Homo sapiens
tgttctcttgttagcagtcatttgttattgattggtaattgccatcatcaaaatgccaaatcatgttgacccatctcttctgcttcaagactttcccagccttgttccagttactatggctgcataacaaattacccagaaccttagtggcttaaaacaatgacaacacttgttttgtttatgattttataattgggaagac

SEQ ID NO: 211 - DNA, 202 bp, Homo sapiens
caaatcatgttgacccatctcttctgcttcaagactttcccagcctgttccagttactatggctgcataacaaattacccagaaccttagtggcttaaaactaatgacaacacttgttttgtttatgattttataattgggaagactccatggaaatggcttatctatgttccactcagcattaactagcaaagtgcagcga

SEQ ID NO: 212 - DNA, 202 bp, Homo sapiens
ggcagttggtactagccattagcagatcccagctaatgcttttggccagaaaattacacgtaacctttccaggtggcctgggcttcctcacaacatggagagctgtgagcatcctgacagagagagcaagggccaactgagtgctgtgttgcttcctatgacctagcttcagaaaacacgtaacattacttcagtgatagtc

SEQ ID NO: 213 - DNA, 202 bp, Homo sapiens
ttccaggaagggaacatagatccatctctcattgcaggtcacagtggaagaagaacatgtgggatggcatctattggtgcagccatctttgaaacacataagtgttccatgaagctcaatcctttgagtttcttgtaagacacttctatttttccagtccatatgcctaaaagaacacataaggcaactgcaaagataggag

SEQ ID NO: 214 - DNA, 202 bp, Homo sapiens
actgagtgggaaactcaaaaatcctcatgggcaagcagacagacaaggaaagcctagagactcactcattattcatctctgtcctgtgtatttatgatagagtatcaagcttgaacgagtcattaaaaatatgtttagaaaactttggtttgaacaaagtaaaatgtttttctatatttcttatcttcttagaaaatttagt

SEQ ID NO: 215 - DNA, 202 bp, Homo sapiens
agggacaacatatgctgctgatggagctcaacttcaccctgcctcattggaccaagttcattacctgagagtgatgaaatgaggcgaaagagaatgtacaagcacgaagactgcagaaacttacaaaaggagtgtaggctagaaatctagaccagcctcaagatgccaagattccacttagagaaaagggaaatacatctgt

SEQ ID NO: 216 - DNA, 203 bp, Homo sapiens
ctatgaaagtcctggaaaattccaagggaagtatttctacctaaaagtatatgaagattgaaacagccaaggaaggaaggagattattgctgttggagagcgtgtggaatttcaaccagagtccagagaagacactccatcctcaagatgcattaaattttatacgaggtcttttgtggactgttcggacacatcctgcctct

SEQ ID NO: 217 - DNA, 202 bp, Homo sapiens
ttggagagcgtggaatttcaaccagagtccagagaagacactccatcctcaagatgcattaaattttatacgaggtcttttgtggactgttcggacacatctctgcctctgggcacttcagattagcttcggtactagagtgtttgatgatttttcaaaaaccatgaatcctctctgacactggcaaagaagcttggaaaag

SEQ ID NO: 218 - DNA, 202 bp, Homo sapiens
cttcagattagcttcggtactagagtgtttgatgatttttcaaaaaccatgaatcctctctgacactggcaaagaagcttggaaaagatgagataacatactgttatttactagctcaatgattgctaagtggtatttttcttcttttgattagattaaaaaatgtatttaaagaatgaaatgtattaaacagaagctattt

SEQ ID NO: 219 - DNA, 202 bp, Homo sapiens
ttcagattagcttcggtactagagtgtttgatgatttttcaaaaaccatgaatcctctctgacactggcaaagaagcttggaaaagatgagataacatacagttatttactagctcaatgattgctaagtggtatttttcttcttttgattagattaaaaaatgtatttaaagaatgaaatgtattaaacagaagctattta

SEQ ID NO: 220 - DNA, 202 bp, Homo sapiens
ctatttacacttaggcaaaagtcctgtttcagagttgggcaagtgttcgctgcaatcaggatcataaccacacctgtacgatttctagcattcattagaaagacctacccagacactgaaataataagattgtcaacttttgccctctctgggatcagtgccaaatgactgattttcaatgtcctggctccatgttgctctc

SEQ ID NO: 221 - DNA, 202 bp, Homo sapiens
ccctctctgggatcagtgccaaatgactgattttcaatgtcctggctccatgttgctctcattgtgatctctcttctgattctttacttcttacttgcttagttattttcctccttatcattctctgcccccaacttccttcttcttctgtctggacttctgtcttagttataagaaataccatagataggggcttgcgaac

SEQ ID NO: 222 - DNA, 202 bp, Homo sapiens
tcttctgattctttacttcttacttgcttgttattttcctccttatcattctctgcccccaacttccttcttcttctgtctggacttctgtcttagttatagagaaataccatagataggggcttgcgaacaacagaaacatatttctcagagttctggagctggaagtctgagatcacagtgcttatgtggtcaggttctg

SEQ ID NO: 223 - DNA, 202 bp, Homo sapiens
tgtagcaggagatacctagaaatacaaagaaatgacaacacacgctttggaattacatggaactgagtttgaatccaggcttctccacacactcacactgcgttggctcacctggttgagcctccgtttctgatctttaaaataatattaataccactcacatttcagtgttgtaaaagcaaatatttccaaattgtaaaca

SEQ ID NO: 224 - DNA, 202 bp, Homo sapiens
cctggttgagcctccgtttctgatctttaaaataatattaataccactcacatttcagtgttgtaaaagcaaatatttccaaattgtaaacattaagttcagatggtgaatgaaaaaattctttgcatggtgtttggtttttggtaaaagctctaacacatgatggctattatcaatattattattaggctgaaagccagta

SEQ ID NO: 225 - DNA, 202 bp, Homo sapiens
gtttttcaccatgcaaagtgtcttcctattgtggggtgtaagtgattctacacccttgagctctgacactgccatgggattggtagcagttatgatgtaaagcagggccttgaaaagtgctttcaggataggatctgtttgctctttcccctctgccactttcatgaggaaaaacatgcccaggacagcacacaggtcacag

SEQ ID NO: 226 - DNA, 202 bp, Homo sapiens
tgagctctgacactgccatgggattggtagcagttatgatgtaagcagggccttgaaaagtgctttcaggataggatctgtttgctctttcccctctgccagctttcatgaggaaaaacatgcccaggacagcacacaggtcacaggaggaggaaaaaaagtcacctggagcagtgacctctgtaacctagtggaggtcaga

SEQ ID NO: 227 - DNA, 202 bp, Homo sapiens
aaaaagtcacctggagcagtgacctctgtaacctagtggaggtcagaactgctcagccaggtgccatcaagatcagcagactctctgtcagcctgtagacagtgttttaatccatgaattccagtgtgatttgctatgtagaattattgtgaccatagatagttgatacaatatatatgtatgtgtgtacatatatagtata

SEQ ID NO: 228 - DNA, 202 bp, Homo sapiens
ctttaattataatatttcatttccttctataagtaattgctacatgatattatgaatatttttaaaaatccaaaaatatattttcccaagataataacctcgtatgtgtacagaatccaacctgacagctaaacattcatacctcaatgatctttgaagaggtatttattttccttagaaagctgggatgacttgcacagtc

SEQ ID NO: 229 - DNA, 202 bp, Homo sapiens
taacagtacaattcattctaaaggaagagaaattgtaagaaattggaggggtgttttaagtcatgcttctcatatatgattaaaaactgatgtcagaggcattggagaaaatacaatcaggttgagaattagtaacttcctttctaattaagaatgtagtagattgaagaagcattgttctcagccctctctacagggactc

SEQ ID NO: 230 - DNA, 202 bp, Homo sapiens
gagaaaatacaatcaggttgagaattagtaacttcctttctaattaagaatgtagtagattgaagaagcattgttctcagccctctctacagggactctactgttttacttaagtaattgatctgaggtagctcagaggcaccttggattttgtgaagattacattcagcttttgccacttggacacccaggactatatttg

SEQ ID NO: 231 - DNA, 202 bp, Homo sapiens
tacattcagcttttgccacttggacacccaggactatatttgtggccaaatttagccaccgtttcgatcctattttattttctgttctctctctcatagtagtatctgtaaattaccttaagtccttttgtaagatatagaacgaataagtaagtaatgaagactgtaagaaagtcattcatcgtccccacctctcaatttt

SEQ ID NO: 232 - DNA, 202 bp, Homo sapiens
accagagtttcaattgtaaacgcctcctctcatttattcccttctaagagtggtttgagcatataaatgcataaataaataaatggctttgtgtgtccaaagatctactggcacacatcaggtcctatgagacatcacattatcaatgatacatttcaattgcttttaaaaaataaccagcaacttttcaggttttagagat

SEQ ID NO: 233 - DNA, 202 bp, Homo sapiens
aggccctgacctgagatcttagtagggggaaaaaatcatctttctaaagagaaaaacatcactgagaagacaatcttaactggatcaacattcatgttgaagaaaatgaatgtgctttaaaaattgaatagatgtttccagagctttggttcctgggaagaaataaaaacaactgttaagccaggtcaatgaggagaggagc

SEQ ID NO: 234 - DNA, 202 bp, Homo sapiens
agagctttggttcctgggaagaaataaaaacaactgttaagccaggtcaatgaggagaggagcagtgaagttattctatatctctagtcatctttgactgatcaaatttattgcatttttttcaataaaaattgtcttttgtttaatcgaatgcttaaacttgtttgcaagcctcaatgagcaggggatggtgggtttccag

SEQ ID NO: 235 - DNA, 202 bp, Homo sapiens
tgttaagccaggtcaatgaggagaggagcagtgaagttattctatatctctagtcatctttgactgtcaaatttattgcatttttttcaataaaaattgtctttttgtttaatcgaatgcttaaacttgtttgcaagcctcaatgagcaggggatggtgggtttccagaattaggctgagagactaagacagacagcttggt

SEQ ID NO: 236 - DNA, 202 bp, Homo sapiens
aatgaacccacttttttgtcagtgattcccatctcctggcaatccctgtgaaattcacactcctgggcagttggagattctactgacataaatcatgtgaagccaagtgtgggacaatgctagagaccaccaggatgaaatatctctgtttcaaactaactcttgttcccaagtagggcctcttatacctgtttcccagagc

SEQ ID NO: 237 - DNA, 202 bp, Homo sapiens
ttagaaagtatttaggatgatatggtatctttccagataaagacaggcctccttctcctttgacaaagtgaattggaggcaatgtagatgttacttgtgtcttgcaccctttgatgtgtagtatagacattgggcttcacctagaagggctcttcaggctgctgggctgacttctctttcccttgctgccctgctccaataa

SEQ ID NO: 238 - DNA, 202 bp, Homo sapiens
taaagccacagctccaatgtcatgtctttggagacactctctcatctccatctctcctatctaaaccattcacagcacaccatcttgttttatgccctttagcagtgtctgtcacatctgtcatttcttctttactaattgtttcttgacaagcagaatatgaactccaagatgacaggagcatctcatatagatggctata

SEQ ID NO: 239 - DNA, 202 bp, Homo sapiens
tccctacttctaataaattctgttctcattgcatatattatttctccatcctttgctatgcccacacataagtgtcctttgtttagcttacacttcaaatactctgtttacttgtcatctgtaggaaatcattcttggctgtcccaaaccggattgggtgcttcctctacacttccttcagttagagcactactcacactgc

SEQ ID NO: 240 - DNA, 202 bp, Homo sapiens
gtgattacctttctacatttgtacataaaacacccatggtgttccacaggaaaggcataagtcatcagcctggtgctttttttatctcctgtttaacctcagtaattaacacctttagagctcttatgttttgccaggctgcagctgaggcttttgcttgggtaatttcccccacgtcagctctaggaagtaggaagtgttg

SEQ ID NO: 241 - DNA, 202 bp, Homo sapiens
gcgtcacacagccggtaagtgctgcgggcagaattggaacccaggcagtcatagatagtaaagtcgtcactgtgaaacacagagaaatggcacaacctcactggcaaatttgatatcaatgccaccagtaatttctgtgcttgctgatttagtgtggttacaaatatcatactttaaaactctgattagggtattctgcttt

SEQ ID NO: 242 - DNA, 202 bp, Homo sapiens
ttctggtttccttcaaaacaaatttactttccatgtatttcaaacttactcattgtttaattctgaagcttgataggctgcttttatttgagctggaggaatttctctccctccatgccttctgtataactgtaggaaaaatatgtcaaagagtagatgagatggtaaacaaataatctgctgcaaaatgcatgaagaataa

SEQ ID NO: 243 - DNA, 202 bp, Homo sapiens
aacttactcattgtttaattctgaagcttgataggctgcttttatttgagctggaggatttctctccctccatgccttctgtataactgtaggaaaaatactgtcaaagagtagatgagatggtaaacaaataatctgctgcaaaatgcatgaagaataaactgggggagtaagcaaagctttaagtaacatgttcttaaat

SEQ ID NO: 244 - DNA, 202 bp, Homo sapiens
actgtaggaaaaatatgtcaaagagtagatgagatggtaaacaaataatctgctgcaaaatgcatgaagaataaactgggggagtaagcaaagctttaagctaacatgttcttaaatatgcaaaacaagaaattgtgttattagctcacattatgagaaatttacactaagttaggaagcttcacataaaataaaaataaat

SEQ ID NO: 245 - DNA, 202 bp, Homo sapiens
gggagtaagcaaagctttaagtaacatgttcttaaatatgcaaaacaagaaattgtgttattagctcacattatgagaaatttacactaagttaggaagcgttcacataaaataaaaataaatctattcagtttatatattctttgtcacctctttggagaaatgcttccttgttgcttccactgaatttcatatttacctt

SEQ ID NO: 246 - DNA, 202 bp, Homo sapiens
tatatattctttgtcacctctttggagaaatgcttccttgttgcttccactgaatttcatatttacctttgttatatgtctcaccactgtggattgtatgctattgttgtatttctctgtctctcatcaaatttctaagatcaggatctggctctttgtgcctaggacatgctttggaacatgatatatctctaaaacaaac

SEQ ID NO: 247 - DNA, 202 bp, Homo sapiens
ggctaagatgtaaaagtgaaaaacgccaagtgtacaagctctttgccagcctatttggctgtgtctcctgaagttaaacatgccaaaaaacccaataattcttactactgagcatatatagtcactaaaaacatgggggttgatggtcatcaaaagacttgttcaagatttacagtatcactatttgttatagtcagaaact

SEQ ID NO: 248 - DNA, 202 bp, Homo sapiens
agcctatttggctgtgtctcctgaagttaaacatgccaaaaaacccaataattctactactgagcatatatagtcactaaaaacatgggggttgatggtcagtcaaaagacttgttcaagatttacagtatcactatttgttatagtcagaaactggaaattactcaaatacccagaaatagaagaatgcgtaaacgaattg

SEQ ID NO: 249 - DNA, 202 bp, Homo sapiens
attataaccactgaattctcaatgtttgcgaagcaaaagatggtacctctaagaatggatgcatccttctaagcatccacatggctaccagtgagtgaacagaggaattctagctcattcagctgaaaacatagtttttgttagcttgccaggggaggcctgattgtcaattgtgcctgaaggaagtgagtgctgttaagca

SEQ ID NO: 250 - DNA, 202 bp, Homo sapiens
cgaagcaaaagatggtacctctaagaatggatgcatccttctaagcatccacatggctaccagtgagtgaacaaggaattctagctcattcagctgaaaacgatagtttttgttagcttgccaggggaggcctgattgtcaattgtgcctgaaggaagtgagtgctgttaagcatagacatttctcctttgtgtacagtgga

SEQ ID NO: 251 - DNA, 202 bp, Homo sapiens
ctttgtgtacagtggaaatgatgggcacacacacctcctgctcacttgcagtgactggattctattcacaagccagtgctgccatctgacaatacttgcacttggaatggccacattcataccatttctttagtaagctgcgaggttatatttcttcctcctgtcacagaagcagaatttgatctgtgtttgttggcgcggg

SEQ ID NO: 252 - DNA, 202 bp, Homo sapiens
tctactgaggaaaattactttttccccccattttcctaaggcaagtaagtgagaatgttaggagttatgatagtgttgaatttcagatcaggtaaactgaacaacgagccctgctgtgaattggagtgttttcttttgaatgttttggcagctctttgaaagcagacagtaagcctcagactgtcagccgttatgtttgata

SEQ ID NO: 253 - DNA, 202 bp, Homo sapiens
atttcagatcaggtaaactgacaacgagccctgctgtgaattggagtgttttcttttgaatgttttggcagctctttgaaagcagacagtaagcctcagaactgtcagccgttatgtttgatattccttgatgtcgatactggatatgaaagaacattgtgcatttggtttaaaaactgtgtgtgtgtgtgtgtgtggtcag

SEQ ID NO: 254 - DNA, 203 bp, Homo sapiens
tgggtgaagggaggaattgatgtggggagaaaaagcaggcaagaaatggtgtattattaaggcgctttccataaaagtgacaaaagtccggggagaaactatcacaaaacaatgcaaaacagacacctcaatattatctcagccagaagacccaatgcccagtcattggctagggctgccctggtaggatgtgaattcacaga

SEQ ID NO: 255 - DNA, 202 bp, Homo sapiens
cccagcagtggtaagaaaaatcaagaacctgtaggtatgcagtgtctgctgtagatatgccttgggatcccctattcatcctttaaaggtcagcctggacctagtttattaaagaattctgctgtgtggaaagggccgcctgaaagtaagatgcccattttggagatgagttcagctattaacatctttgagaagggccaag

SEQ ID NO: 256 - DNA, 202 bp, Homo sapiens
tcaagaacctgtaggtatgcagtgtctgctgtagatatgccttgggatcccctattcatcctttaaaggtcagcctggaccagtttattaaagaattctgactgtgtggaaagggccgcctgaaagtaagatgcccattttggagatgagttcagctattaacatctttgagaagggccaagggccaagttatatgcccaac

SEQ ID NO: 257 - DNA, 202 bp, Homo sapiens
ctggaccagtttattaaagaattctgctgtgtggaaagggccgcctgaaagtaagatgcccattttggagatgagttcagctattaacatctttgagaagaggccaagggccaagttatatgcccaaccatgttttcagcttgtaaaattattctagaaggaagggatttagtgagtcagtggacaccttcagaaagagtgt

SEQ ID NO: 258 - DNA, 202 bp, Homo sapiens
tctaggggaggtgagttaaccttttttggtgataatttcctgacaaaggatgcctaaaaaccctggaaaatggtaatagttcatctactgttcctgcagcctgctggactgggatcttaagcagtctgacccattttatactcatatccttaatacgttgcttcacacaatgctttttggcattaatggacatgatcaggaa

SEQ ID NO: 259 - DNA, 202 bp, Homo sapiens
ctaggggaggtgagttaaccttttttggtgataatttcctgacaaaggatgcctaaaaaccctggaaaatggtaatagttcatctactgttcctgcagccagctggactgggatcttaagcagtctgacccattttatactcatatccttaatacgttgcttcacacaatgctttttggcattaatggacatgatcaggaag

SEQ ID NO: 260 - DNA, 202 bp, Homo sapiens
tggttaactacaaaggggtagacagggatgttcacggctgaggaaactgttctgcatcttaattgctgtgtcagttacaggactaggtgcatttgtcaaaattaaggtaaataaattacatctcaataaactcgactcacacccaccaaaaaaataataagaaagaaaaaatggtagagccatgttaggtcaccaaggtaga

SEQ ID NO: 261 - DNA, 202 bp, Homo sapiens
gttctctcaatatccctaagtttaatggctcaatatttagcatgcgtttcataactccagctttttttgggtggaggtaggtagggaaaatgggaatatacgtaaagagttttcaaaacacttgctatttgtaaggcttctttttttatgtaccataacgcacacagccctctcaggcatggggtcaggctgtttccttgga

SEQ ID NO: 262 - DNA, 202 bp, Homo sapiens
gtttcataactccagctttttttgggtggaggtaggtagggaaaatgggaatatagtaaagagttttcaaaacacttgctatttgtaaggcttcttttttatatgtaccataacgcacacagccctctcaggcatggggtcaggctgtttccttggagtggaattgaacacgaatgaggaatagtgagaaactgggttaggc

SEQ ID NO: 263 - DNA, 202 bp, Homo sapiens
cacagccctctcaggcatggggtcaggctgtttccttggagtggaattgaacacgaatgaggaatagtgagaaactgggttaggctaccatggaaaatatagagagccctggaaagcgtcattacaaggtctgacaatgattccagggttactggagaaacttttttttttctcttttccctctttcctccaagtacccaga

SEQ ID NO: 264 - DNA, 202 bp, Homo sapiens
gcatggggtcaggctgtttccttggagtggaattgaacacgaatgaggaatagtgagaaactgggttaggctaccatggaaaatatgagagccctggaaaagcgtcattacaaggtctgacaatgattccagggttactggagaaacttttttttttctcttttccctctttcctccaagtacccagatccctccaacagga

SEQ ID NO: 265 - DNA, 202 bp, Homo sapiens
tcaggctgtttccttggagtggaattgaacacgaatgaggaatagtgagaaactgggttaggctaccatggaaaatatgagagccctggaaagcgtcattaccaaggtctgacaatgattccagggttactggagaaacttttttttttctcttttccctctttcctccaagtacccagatccctccaacaggatcccagaa

SEQ ID NO: 266 - DNA, 201 bp, Homo sapiens
ggaatagtgagaaactgggttaggctaccatggaaaatatgagagccctggaaagcgtcattacaaggtctgacaatgattccagggttactggagaaacttttttttttctcttttccctctttcctccaagtacccagatccctccaacaggatcccagaagtgtgacactaacaatgaattttcaatgcatcctgcac

SEQ ID NO: 267 - DNA, 202 bp, Homo sapiens
gagacaagggctaccgcagagaggcagagaggctggaaagccaattgtcatggatatagatggtcatcgtttctgtctttggctctaccatatgctagctcttgtggctttgagcaagctacctaacttctctaagcctcaaaattttaacctgtgaagtagagataataatagcactaccccaacatgtgattgagggtta

SEQ ID NO: 268 - DNA, 202 bp, Homo sapiens
taacctgtgaagtagagataataatagcactaccccaacatgtgattgagggttaaatgagaaatccattcaaagcacatgaatagtgcctggctcatagctaagtggtcataagtggtagccatgacagtaatttcatgggaagagagatcacctcaggaaagcttcaaagcatcagggatgtttaagctgagtcttcaag

SEQ ID NO: 269 - DNA, 202 bp, Homo sapiens
agaaatccattcaaagcacatgaatagtgcctggctcatagtaagtggtcataagtggtagccatgacagtaatttcatgggaagagagatcacctcaggagaagcttcaaagcatcagggatgtttaagctgagtcttcaaggaaaagtaagtacaagctacatagacccagtgtacccgcttgtggaactgggaggacat

SEQ ID NO: 270 - DNA, 202 bp, Homo sapiens
ccatgacagtaatttcatgggaagagagatcacctcaggaaagcttcaaagcatcagggatgtttaagctgagtcttcaaggaaaagtaagtacaagctactatagacccagtgtacccgcttgtggaactgggaggacatttcaagcagagggagaaggatatgaaaagggttgtaggtggaaaatatcctgataagttca

SEQ ID NO: 271 - DNA, 202 bp, Homo sapiens
agcatcagggatgtttaagctgagtcttcaaggaaaagtaagtacaagctacatagacccagtgtacccgcttgtggaactgggaggacatttcaagcagacgggagaaggatatgaaaagggttgtaggtggaaaatatcctgataagttcacattaagaccagattaagatctctggggaatctaaagaataatggaacc

SEQ ID NO: 272 - DNA, 202 bp, Homo sapiens
caaatgtattctaatatcagtgttaaatctgaaagtaactgggaggcacatttctgatttgaccttcaacaactaggttgaatgctttgtttcttcaagaagatttttgcttttgtgagtacctaggaatgttgaatctccttgtagggtcatccagggacctcagagttacctatgaatggagtgggcaaggattttggcc

SEQ ID NO: 273 - DNA, 202 bp, Homo sapiens
agggtcatccagggacctcagagttacctatgaatggagtgggcaaggattttggccaagactgaaatatgagcagaaaccaaactgtgaagggtcgttgcttgggggctttctgaggagttgtgcagggaaagtttggtgaaaaatttcaggctggttgcgtgacatgcaaaggtaactctggtggccttacaaggaatgg

SEQ ID NO: 274 - DNA, 202 bp, Homo sapiens
gctggagtgcggtggcacaatcttggctcactgcaagctccacctcccgggttcacaccattctcctgcctcagcctcctgagtagctgggactacaggcagcctgccaccacgcctggccaattttttgtatttttagtagagacggggtttcacggcaaatcttatttttacttttattttttgtagtccagaattacag

SEQ ID NO: 275 - DNA, 202 bp, Homo sapiens
ccaattttttgtatttttagtagagacggggtttcacggcaaatcttatttttacttttattttttgtagtccagaattacagcaccatagagacgaaatattacaggtgagggcgggagcttctccactcagtctgtgcatgtgggagggtgggcaacttcctgccagccttctggtctcatatgcatttggtgaggatgt

SEQ ID NO: 276 - DNA, 202 bp, Homo sapiens
tagagtgaaatcagacttaatggagtgtggccacctaaagacttcagagctagcagaagcgatcagcaatctactagatgagtgatttggtgggggtgtcgtttactattctttcatgccatggaacaatacatagtattgtcatgtcaggtttaatgattcagcaatttaatatccttgacttttttgtggagttattatt

SEQ ID NO: 277 - DNA, 202 bp, Homo sapiens
ggggtgtcgttactattctttcatgccatggaacaatacatagtattgtcatgtcaggtttaatgattcagcaatttaatatccttgacttttttgtggaagttattatttagttgggaatatttttcaggacccagaagcattatgtttttgatatgtttggaacttgatctaagatgtacttcagacaccagacagtgtt

SEQ ID NO: 278 - DNA, 202 bp, Homo sapiens
catagtattgtcatgtcaggtttaatgattcagcaatttaatatccttgacttttttgtggagttattatttagttgggaatatttttcaggacccagaaagcattatgtttttgatatgtttggaacttgatctaagatgtacttcagacaccagacagtgttttatgttccatgacgactctgaaattcatgcagctttt

SEQ ID NO: 279 - DNA, 202 bp, Homo sapiens
tgtggagttattatttagttgggaatatttttcaggacccagaagcattatgtttttgatatgtttggaacttgatctaagatgtacttcagacaccagacgagtgttttatgttccatgacgactctgaaattcatgcagcttttgttgtctgaactttcatttgatctgtgttaattttttgtattaatagcttgcctct

SEQ ID NO: 280 - DNA, 202 bp, Homo sapiens
tcatttgatctgtgttaattttttgtattaatagcttgcctcttcattttcattattactgacaaatatttacttagtatctactacatataaaatatcagtgtaagtattctgtgtatgttcaacttgaaaaactttctaatttggcaaaacatagagactaatttaacaaatacccatgtagtaggatttcagaattaac

SEQ ID NO: 281 - DNA, 202 bp, Homo sapiens
aaaaactttctaatttggcaaaacatagagactaatttaacaaatacccatgtagtaggatttcagaattaacaaatgccaacatttaatcatattttctctgagataaacaatgtatgtgtgagtaaaagaaataaacatcactggcaaaattcaattatttttacttcctaagtttttatacaaaatggctaacaattta

SEQ ID NO: 282 - DNA, 202 bp, Homo sapiens
aacaatttaagtttttgaatagaaggattcaatatttacactacttaatcacactgttcctggtatctatgcttggtacatagtagttgctcaataaacactgtgttgagatgttttgtccaattcttacatagccaggattgactcctatgcccactcttctatactttttatacattttatttgagagaatacatttact

SEQ ID NO: 283 - DNA, 202 bp, Homo sapiens
taaaaattacacccaactcataggtttattgcggggcttagttgaactaatgcatgtgcttactgtttagtaagtgctaagtaaatgttagttagtgttacttccttttattgttttttaaccaacacctttcttcatgggttcacagtccactgcagaagcaaacagaaatatttacaacttcaaagtgctgtgatagacg

SEQ ID NO: 284 - DNA, 202 bp, Homo sapiens
acacctttcttcatgggttcacagtccactgcagaagcaaacagaaatatttacaacttcaaagtgctgtgatagacgttagtgcaaagaaatgtagataacgggagtgaacagggaagtgaggaaggcttcacaatggagaggtgaactttgattcattttctggagaagaaatctagtttcactaagtgaggaaagaggt

SEQ ID NO: 285 - DNA, 202 bp, Homo sapiens
aagcaaacagaaatatttacaacttcaaagtgctgtgatagacgttagtgcaaagaaatgtagataagggagtgaacagggaagtgaggaaggcttcacaagtggagaggtgaactttgattcattttctggagaagaaatctagtttcactaagtgaggaaagaggtcgttgcaggcagagggagtagtatatgcaaaaga

SEQ ID NO: 286 - DNA, 202 bp, Homo sapiens
aagaatagtagaccaaagagtgtacaactccatgtgtttgtgcgcatcggggcctgcttctttattgaaacttacattcaaacgttaaatattattacacctatgtaagaaaagacgtatgagtaatgctcttccaccctcctccccctcacgttcccattcctgcagcactagaaacccactgatatgtcttcaagggaaa

SEQ ID NO: 287 - DNA, 202 bp, Homo sapiens
atgccactaacgttgatgaccaggaccatggagacattggcaaggaatttacagcttctcaagttccaataatcttattatagtctagtgtatgcaatacagccttcttacacatctgtcatctagctggacccagtgggaaggttcatggtctttggactcagagagagcaatgctacaaccctggctttgttttgctgat

SEQ ID NO: 288 - DNA, 202 bp, Homo sapiens
cctggaccccttctctcttttcttctgccctgctgttgcaccaagaggttgacctcagggactacattccctggctcctttatcctttggctttgattgtagtataggcaatgagagggtctagcaggatgcaggatggcagtaagagggagaggctgggatatttcttcctcttgcttccgccttgcttgtctgtggttct

SEQ ID NO: 289 - DNA, 202 bp, Homo sapiens
gcaccaagaggttgacctcagggactacattccctggctcctttatcctttggctttgattgtgtataggcaatgagagggtctagcaggatgcaggatgagcagtaagagggagaggctgggatatttcttcctcttgcttccgccttgcttgtctgtggttctaatggtggctgtgctcctccatgctccagctccagac

SEQ ID NO: 290 - DNA, 202 bp, Homo sapiens
ttatcctttggctttgattgtgtataggcaatgagagggtctagcaggatgcaggatggcagtaagagggagaggctgggatatttcttcctcttgcttcctgccttgcttgtctgtggttctaatggtggctgtgctcctccatgctccagctccagacaggtgtcccatctcctttgcttctgccttgctccagtgacat

SEQ ID NO: 291 - DNA, 202 bp, Homo sapiens
ctcccttgcttttgacagttccagtgcctggccattccctgttggttgttttagctctgcccacagatccataaggagcccctatattaagttattctcaagttaaatcctttcaatatgtcatctctttcctgctgagacccttaccaaatcaactacttactacatgactttgtccaagggccataatcttttatggcct

SEQ ID NO: 292 - DNA, 202 bp, Homo sapiens
gagacccttaccaaatcaactacttactacatgactttgtccaagggccataatcttttatggcctcaggttctccacatgcaaaatcaggataataatgcttctctacctcatgaggttatttaaaggaaacggtgagatcatgtgttttgatagaatatattttaaagcctctatcatggttcttgacacataggaggtg

SEQ ID NO: 293 - DNA, 201 bp, Homo sapiens
tacatgactttgtccaagggccataatcttttatggcctcaggttctccacatgcaaaatcaggataataatgttctctacctcatgaggttatttaaaggaaacggtgagatcatgtgttttgatagaatatattttaaagcctctatcatggttcttgacacataggaggtgctcaggaaatattaggtactattgtca

SEQ ID NO: 294 - DNA, 202 bp, Homo sapiens
actttgtccaagggccataatcttttatggcctcaggttctccacatgcaaaatcaggataataatgttctctacctcatgaggttatttaaaggaaacgagtgagatcatgtgttttgatagaatatattttaaagcctctatcatggttcttgacacataggaggtgctcaggaaatattaggtactattgtcattgcaa

SEQ ID NO: 295 - DNA, 202 bp, Homo sapiens
tgggccattgccttggatgacctgctcattttatgtttgaggaaactgaggcccagcatgcttgtccaagttcacacagttaattagtggtggagctaagagatagtatgagtctacattgaaaggaggccagctgtgtttacatcaattctatttaacttctttaattaagctgttgattccttcagtgacgtcacccact

SEQ ID NO: 296 - DNA, 202 bp, Homo sapiens
tctttaattaagctgttgattccttcagtgacgtcacccacttgttttagaaatgagttacataagcgcgtctccctccactatttatttgctccctctgatgaagagtatcgggctgtcccaaaataagcaaagattcgagagaatacccctgtttcattgcttctgctccctgctgctgactcatttactcctgggcctc

SEQ ID NO: 297 - DNA, 202 bp, Homo sapiens
ttaagctgttgattccttcagtgacgtcacccacttgttttagaaatgagttacataagcgcgtctccctccactatttatttgctccctctgagaagagctatcgggctgtcccaaaataagcaaagattcgagagaatacccctgtttcattgcttctgctccctgctgctgactcatttactcctgggcctctgagctc

SEQ ID NO: 298 - DNA, 202 bp, Homo sapiens
aagcgcgtctccctccactatttatttgctccctctgagaagagtatcgggctgtcccaaaataagcaaagattcgagagaatacccctgtttcattgctctctgctccctgctgctgactcatttactcctgggcctctgagctcgctcacatcaatcacccatcctcagaagaacacactgcgtccggcctcacaggccc

SEQ ID NO: 299 - DNA, 202 bp, Homo sapiens
ctttctcttgtttcttttcttatgagtaacatgtgatcttttaaaaattattgtagtaagaacacctaacatgaaatctaccctcttaacaaatttttaagttgtacatgagcctttaaaacaaaacaacctgactttttaatgatcgccattctaactggcgtgagatggtatgtcattgtggttttgatttgcatttctc

SEQ ID NO: 300 - DNA, 202 bp, Homo sapiens
acacctaacatgaaatctaccctcttaacaaatttttaagtgtacatgagcctttaaaacaaaacaacctgactttttaatgatcgccattctaactggcagtgagatggtatgtcattgtggttttgatttgcatttctctaatcaccagtgatgatgagctttttttcatatgtttgttggccgcacaaatgtcttcttt

SEQ ID NO: 301 - DNA, 202 bp, Homo sapiens
tcttgtaaatttgtttaagttccttgtagattctgtatattagccctttgtcagatggatagattgcaaaaattttctcccattctgtaggttgcctgttctacaatggatgccagagaggatgtggagaaataggaacacttttacactgttggtgggagtgtaaatgagttcaaccattgtggaagacagtatggtgatt

SEQ ID NO: 302 - DNA, 202 bp, Homo sapiens
tttaagttccttgtagattctgtatattagccctttgtcagatggatagattgcaaaaattttctcccattctgtaggttgcctgtttacaatggatgccaggagaggatgtggagaaataggaacacttttacactgttggtgggagtgtaaatgagttcaaccattgtggaagacagtatggtgattcctcaaggatcta

SEQ ID NO: 303 - DNA, 202 bp, Homo sapiens
gatggatagattgcaaaaattttctcccattctgtaggttgcctgtttacaatggatgccagagaggatgtggagaaataggaacacttttacactgttggttgggagtgtaaatgagttcaaccattgtggaagacagtatggtgattcctcaaggatctagaaccagaaataccatttgacccagcaatcccattactga

SEQ ID NO: 304 - DNA, 202 bp, Homo sapiens
actgttggtgggagtgtaaatgagttcaaccattgtggaagacagtatggtgattcctcaaggatctagaaccagaaataccatttgacccagcaatcccagttactgagtatatatccaagggattataaagcattctactatacagacacatgcacacatatgtttattgcagcactattcacaatagcaaagacttgga

SEQ ID NO: 305 - DNA, 202 bp, Homo sapiens
gaaagggaaggaaggaaggccacatgaatggatgagtggttacccttatgagcaactgaggcttaatcccaccatggaccctctaagacagtggtcatcaagcttttttggcatcagggaccagtttcatggaagacagtttttccacggactggggtggtgggggatggtttggaggtgattcaagtgcattatgtttatt

SEQ ID NO: 306 - DNA, 202 bp, Homo sapiens
tgattacattgtaatatataatgaaataattatacaactcaccataatgtagaatcagtgggagccctgagcttgttttcctgcagctagaccatctcatcgtgggggtgatgggagacagtgacagatcatcagggattagattctcataaggagcacacagtctagatccctcctatgagcacttcacagtagggtttgc

SEQ ID NO: 307 - DNA, 202 bp, Homo sapiens
cagaacttaggcagtaatgggatcgatggggaatggcttaaatacagatgcagcttcactcactcaccttctactgtgcagcctggttcctaacagaccactagactggtatcagtctgtggcccaggggttggggagcccagttctaagagactgtgtggaacatgctcagagctgtcccagtgaggggcaaagaacttgc

SEQ ID NO: 308 - DNA, 202 bp, Homo sapiens
ggggttggggagcccagttctaagagactgtgtggaacatgctcagagctgtcccagtgaggggcaaagaacttgcggtattcctgccatgttaatttccagtacctcagaagttacgtgttgctcctagggcaccaatgccttggcacatgggtcaagcacagtcctgtggccagagagcaacctcaggcagagagacaca

SEQ ID NO: 309 - DNA, 202 bp, Homo sapiens
tacctcagaagttacgtgttgctcctagggcaccaatgccttggcacatgggtcaagcacagtcctgtggccagagagcaacctcaggcagagagacacaaggagaccatctgtgtgttgagaactactgcagatgacctcaggtgacccaaaagggtatgatgggccctgctagctggaatactcagagatgtgggtagat

SEQ ID NO: 310 - DNA, 202 bp, Homo sapiens
gagagttaaatagcaaaagaataatccagaaggctgggtgtccaaccgcattccaggcccagaatcattattttgtgtcgtttacataaattctcagttactgaatgattccattttaggacatgaataggctgaacttctactgcatatacaagtttttccagccttttccatctccctgaattagttcattgtgaaatag

SEQ ID NO: 311 - DNA, 202 bp, Homo sapiens
cataaattctcagttatgaatgattccattttaggacatgaataggctgaacttctactgcatatacaagtttttccagccttttccatctccctgaattatgttcattgtgaaataggttgcctgatggctgaaaaagacaaaaaacttcaagagttttggcactcctcaagtcttccatgccgcctctgtttgttgtata

SEQ ID NO: 312 - DNA, 202 bp, Homo sapiens
tgtgtgtgcatatgtatgtgtatgtttatacatctagcttcttcccccattctcccaccaacactgtttgggtagccagccccccatcctctctaaagccctccatgccctgcagtaaacactgaggccagggctgtgtggcaggactcccagccaagcaatagtaactcctgctgctgccttgagtaaccaggaattctcc

SEQ ID NO: 313 - DNA, 202 bp, Homo sapiens
tgtgcatatgtatgtgtatgtttatacatctagcttcttcccccattctcccaccaacactgtttgggtagccagccccccatcctctctaaagcccccactgccctgcagtaaacactgaggccagggctgtgtggcaggactcccagccaagcaatagtaactcctgctgctgccttgagtaaccaggaattctccatgt

SEQ ID NO: 314 - DNA, 202 bp, Homo sapiens
cactgaggccagggctgtgtggcaggactcccagccaagcaatagtaactcctgctgctgccttgagtaaccaggaattctccatgtctcatgagtcctgcttcattgggctgacaaatggtgactgagaaatactcgccttttgtggtgtcctttgttgtttcatgtgtaatatgccctaagcattttatgtccaaagaga

SEQ ID NO: 315 - DNA, 202 bp, Homo sapiens
ctattcaagtggcaatgtcttatggttaattaagtctggtgttccgttttagaggtgaaaactgagaccaagggaactgaagtggcttggtcaaagccacagtggactcttgggctctggaccctgtctcctggctctctcttttgcaacaggacgcttgtgttccctctctggtcttgccagcagacactccctctgctag

SEQ ID NO: 316 - DNA, 202 bp, Homo sapiens
cttcctgtgcctcctagcttccgtgatgtgagagagaagttggagtgtttgtctccacattccttcgggtcagttctggtgttgacagtgatcacaccttctgctaatggccatgaccccagtcagtggcctccctcccgtggccgcagctctttctgggttctgctggcattgacccttcccctctgctctaagcctgggg

SEQ ID NO: 317 - DNA, 202 bp, Homo sapiens
cacaccttcgctaatggccatgaccccagtcagtggcctccctcccgtggccgcagctctttctgggttctgctggcattgacccttcccctctgctctaaggcctggggatggtacaggctcccgctgttgctagcccctggtggcttagccccctgttgcttcgcttcatcctgtccacacctctgcatgcagtcacttc

SEQ ID NO: 318 - DNA, 202 bp, Homo sapiens
ccgttcagcatctgagtggactgggaacttcctgccaggccagaaggatatgctgaagggtttcttcctttgacaaatggggataacaacagtctactcggtggttttagtgaggatcaaatagggtcatgtgtgcaaatgtgctgtgtctgtctggcccagatgaaggtttgtcttcaccagggatcatataaggattttc

SEQ ID NO: 319 - DNA, 202 bp, Homo sapiens
gtggactcatcccttgatctggaagcaaaactgggagacgtcacacattccagcttcatacctcaggcagctgccagcattgatctgcgaggagggccctagcctcttctgtctccacctgttacttgaaagccagagaagagtgactcatcagcggcctaatgcagcaaagaagacaaataagtctcagcaggtagggtgt

SEQ ID NO: 320 - DNA, 202 bp, Homo sapiens
tctgtctccacctgttacttgaaagccagagaagagtgactcatcagcggcctaatgcagcaaagaagacaaataagtctcagcaggtagggtgtccccaagccatgctccactcagagttctgagcagcagggtcagcctagggaaaaaggcaagtgggaaaagtctactccaagggcctcgtgccccaccccgctctgcc

SEQ ID NO: 321 - DNA, 202 bp, Homo sapiens
gcaagtgggaaaagtctactccaagggcctcgtgccccaccccgctctgccagtctctgcactccgacatgtagctccccccatccgcactctcacctgcctccatctccagaatactaatcctcaatcctctaaggacttaaccaaagaacatcccacatccttttaatgacagcctattaaacttggtaaaattctgata

SEQ ID NO: 322 - DNA, 202 bp, Homo sapiens
cgctctgccagtctctgcactccgacatgtagctccccccatccgcactctcacctgccccatctccagaatactaatcctcaatcctctaaggacttaacgcaaagaacatcccacatccttttaatgacagcctattaaacttggtaaaattctgataacatgtgtgagtgcctaaaacatgctggtactatctcagatg

SEQ ID NO: 323 - DNA, 203 bp, Homo sapiens
gagtgcctaaaacatgctggtactatctcagatgcaggaaagacaaaaatgatcgagacatgttgtcatttcatctctgccaacaaataccattgtgattatctgtgaaattaactgattacttaaaataagcattccaaacaacctgctgatcagagggggccctggtagttggctctagctgtgctttgttagtggttgag

SEQ ID NO: 324 - DNA, 202 bp, Homo sapiens
aaaaaagctgattttgcacatctctcccaactctgtgttcaccagcatcacattggtagtttaaaatctatgacgggccgggcacggtggctcatgcctggtaattctagcactctgggaagccaaggcaggcagatcacttgaggtcaggagttccagaccaacttgaccaacatggcaaaaccctgtctctacgaaaaat

SEQ ID NO: 325 - DNA, 202 bp, Homo sapiens
gcaggcagatcacttgaggtcaggagttccagaccaacttgaccaacatggcaaaaccctgtctctacgaaaaatacaaaaaggaaaaattagtgggcatcggtggcaggcacctgtagtcccaactacacagaaggctgaggcaggagaattgcttgaacccaggaggcggaggttgcagtgagccgagaccgcaccactg

SEQ ID NO: 326 - DNA, 202 bp, Homo sapiens
aaaattagtgggcatggtggcaggcacctgtagtcccaactacacagaaggctgaggcaggagaattgcttgaacccaggaggcggaggttgcagtgagcctgagaccgcaccactgggctccatcaggaagacagaataagtgagactccgtctcaaaaacaaaacaaaacgaaaaaatttctatgatgtagtgaagattt

SEQ ID NO: 327 - DNA, 202 bp, Homo sapiens
tgacctggagggaggaagacccttccaaacccctatccagactcctataagactcagatttttaaaacaaagcaaaacacaaaagcaactctttgcttgcaggatgactcaacttcctttttggtaggctcaactctgcttcccaggaatcagcagtgattagtgatagaaagagtagccctcatgaccccaggtgaccatc

SEQ ID NO: 328 - DNA, 202 bp, Homo sapiens
tccaaacccctatccagactcctataagactcagatttttaaaacaaagcaaaacacaaaagcaactctttgcttgcggatgactcaacttcctttttgggtaggctcaactctgcttcccaggaatcagcagtgattagtgatagaaagagtagccctcatgaccccaggtgaccatcaggacgctagggcaggaaggagc

SEQ ID NO: 329 - DNA, 202 bp, Homo sapiens
tttaaaacaaagcaaaacacaaaagcaactctttgcttgcggatgactcaacttcctttttggtaggctcaactctgcttcccaggaatcagcagtgattaggtgatagaaagagtagccctcatgaccccaggtgaccatcaggacgctagggcaggaaggagcctgttgattgagtgctgacctgcaaaattaccagcac

SEQ ID NO: 330 - DNA, 202 bp, Homo sapiens
gcggatgactcaacttcctttttggtaggctcaactctgcttcccaggaatcagcagtgattagtgatagaaagagtagccctcatgaccccaggtgaccagtcaggacgctagggcaggaaggagcctgttgattgagtgctgacctgcaaaattaccagcacaggtgtccaacaatttcattcttgagaagccagagttg

SEQ ID NO: 331 - DNA, 202 bp, Homo sapiens
ggcaggaaggagcctgttgattgagtgctgacctgcaaaattaccagcacaggtgtccaacaatttcattcttgagaagccagagttgagtttctggggtatgctttggagggttcaggacatgctgcagggtaataggaaggacatggggtgagaaaaaggttcctgccaagttgctgaaagacacatgatagctcaacag

SEQ ID NO: 332 - DNA, 202 bp, Homo sapiens
acttttgaaaatgtgtgctttcccacctagttaaggcaaagttgcctcaaagacaaatatcacattagcacagatgctcttataagagcaattaccatcaagtggtcactgcctggaagttggacctggaaatgacatgctggatggtcatagtcttgttagatgaggtgtgcgtgtgatgataagtgggaaacagcttgat

SEQ ID NO: 333 - DNA, 202 bp, Homo sapiens
ctggaagttggacctggaaatgacatgctggatggtcatagtcttgttagatgaggtgtgcgtgtgatgataagtgggaaacagcttgatttaagggacacttttagtccatggccagcacagtaatgatcttgggaagcctgaagctatgtcttctgttgagcaataaggtacagggcgcagaaggggcaaaataagtcag

SEQ ID NO: 334 - DNA, 202 bp, Homo sapiens
agatgaggtgtgcgtgtgatgataagtgggaaacagcttgatttaagggacactttagtccatggccagcacagtaatgatcttgggaagcctgaagctactgtcttctgttgagcaataaggtacagggcgcagaaggggcaaaataagtcagggagtccagttaacatgtgagatggaaacttaaggatagggacagagc

SEQ ID NO: 335 - DNA, 202 bp, Homo sapiens
tgtgcgtgtgatgataagtgggaaacagcttgatttaagggacactttagtccatggccagcacagtaatgatcttgggaagcctgaagctatgtcttctgtttgagcaataaggtacagggcgcagaaggggcaaaataagtcagggagtccagttaacatgtgagatggaaacttaaggatagggacagagcacgcacaa

SEQ ID NO: 336 - DNA, 202 bp, Homo sapiens
agcaaaaggggtcgaaaacgggaaggacactcagagggcaggaactatgacggggctctgcggccttgtgggcagcagggagagtgtatttttggcctgcagggtctcgtgagctggaagaaatcaggaaagggaagctgagcatttaatcctacatagatcacaggctaatcgtgcccatttagtgtttgaaagtgagaag

SEQ ID NO: 337 - DNA, 202 bp, Homo sapiens
gtgggcagcagggagagtgtatttttggcctgcgggtctcgtgagctggaagaaatcaggaaagggaagctgagcatttaatcctacatagatcacaggcataatcgtgcccatttagtgtttgaaagtgagaagttgtcaaggctttgtgacagccttgaaaccgcaggcattagaggatagaaatcctgagcccagaggg

SEQ ID NO: 338 - DNA, 202 bp, Homo sapiens
aagtgagaagttgtcaaggctttgtgacagccttgaaaccgcaggcattagaggatagaaatcctgagcccagagggaggcaggcactcccatctgcccaagtttggtcttcaacagcttccaactcttcaatattcccaagcagaatcagattaagcagaagtgctcataatagttctccaatttagcctgtatcttctct

SEQ ID NO: 339 - DNA, 202 bp, Homo sapiens
tgataaaaactgcacagggaagttgacacagtcagatattaagttgatatgcctatcattgaatgtctggagcaaggtaacctcacctcagacttgattccttaacaaacttcagtgaatattcaatttttgttttatcacaaatgtattattacacctgcctatatgggaaggtgaagatgggccagttgtttgctttgaa

SEQ ID NO: 340 - DNA, 202 bp, Homo sapiens
tgtttgctttgaaaggaggaaagagtgagaatgtacaactgaatgaaaaaaaatattttggccacagacactttttctcttggctttacaataataaaatagtgtagtgggatttttattttgctttagaatttttagttgttttttcctttttgaagtgctccttcagaggaaacaaatctttctattgacattgaaaatg

SEQ ID NO: 341 - DNA, 202 bp, Homo sapiens
caaaatgcatatgggtcaactatgtttattgcagaaacactagagagtggatgagagatgggtcagtgggcacagaagcccacctgcaactgcactcacaaccttggcctacattgccaaagagaccaatatatcagcaattgattggaaataccacaaagatgaatcacagatcaggaaagcaaatgccaatcaatgattg

SEQ ID NO: 342 - DNA, 202 bp, Homo sapiens
actatgtttattgcagaaacactagagagtggatgagagatgggtcagtgggcacagaagcccacctgcaactgcactcacaccttggcctacattgccaagagagaccaatatatcagcaattgattggaaataccacaaagatgaatcacagatcaggaaagcaaatgccaatcaatgattgagcctaggcaataatata

SEQ ID NO: 343 - DNA, 202 bp, Homo sapiens
tggaaggactctatcagcagcacctgggctccatttgattttttaacctgcttagtatttccaaccgtaatgaccaaaatgtctgccttcagttacagtactttgtaccctgaggaaatgaaataatttagccccctggccctccaccccactccgttttctcagctgcaaacaaagggattggactttatctggttgcttt

SEQ ID NO: 344 - DNA, 202 bp, Homo sapiens
tcaaggattctttttctgttttgtgagatacctggtttttagattcctctccctttttaatttttctccagttcttcctgctttctgcatcctcttcctgacctttccattgttttctctttaacaatcacacactagaaaatatgtagaagtgagtgaaaaatacatctcatgctctgtttagtgtatcctcaaaaagact

SEQ ID NO: 345 - DNA, 202 bp, Homo sapiens
ttactacaactctggctgtgatttatttttctatcccagatatggtgaaaaataatactaatatgtgtgttttttgtttttagctgggtattagcaaaaaatcattccattttccctgagacatcttgctccaaccaccttgcagtgcagctattttgattacttcatgtcactattaatcatgtgcttaaaggaaatagat

SEQ ID NO: 346 - DNA, 202 bp, Homo sapiens
gagaaatagaagtcaatatttgtaatgtgagtcacaggctcaaaatagaaaagtaaaacacatcactgctacagttcccagagggctgtgggttacacagctgcagcagatctgtgaaccaggcctcaccaccctctaggtctattttaaaatttcctctggccccttcctctaattcctaaattccagagcatgatattct

SEQ ID NO: 347 - DNA, 202 bp, Homo sapiens
atccagccctggtgaagtcatagcttgttttgagtgggtcaatggaaaaaaaagatttttttccctagttgtgtataaccttcctctctagtgacgtggcagtggaacgaacttatcatgattttattttgtagtccatacttcattctacagtggctatcactgtttaaaacttcatctcttatgtgttgcctattttgaa

SEQ ID NO: 348 - DNA, 202 bp, Homo sapiens
ccctggtgaagtcatagcttgttttgagtgggtcaatggaaaaaaaagatttttttccctagttgtgtataaccttcctctctagtgacgtggcatggaactgaacttatcatgattttattttgtagtccatacttcattctacagtggctatcactgtttaaaacttcatctcttatgtgttgcctattttgaagaaggc

SEQ ID NO: 349 - DNA, 202 bp, Homo sapiens
agttaccaatgcccaaccagtatctgcttcctcatcagcacgatggtcatagccccatctccctcgcagcatgttttcagaagtaattaatgcttgtgagagtgttttgagatcactgataaaagacattctacctgcccagggtgttattacattatcttattttcctcttcatagtcaggatcaaacccacgataactta

SEQ ID NO: 350 - DNA, 202 bp, Homo sapiens
aatggtaaatcattaagagcaaggcatccactgacgttgcccatcaaaatggacttagtacctggccccagaaatgcccaaactacacatgaccttttgcagcatttatttctgtctccttagaggactgtgtatctctttgaagaataggagaatagacaggatatatagcaactggtaatttcagtttatgatgctcatg

SEQ ID NO: 351 - DNA, 202 bp, Homo sapiens
tgaaagcttacgtggttattaagagtacttccatcagatctttcgaaagaaaggtggagatgaccaaagggcagaaggtcacctacgtaataagatccacatgagacaataattaaaatggaatgcttaaggaggatgcacgtgtcctgaccatataattctcgataaaattactctaaattgatctccatttaaatgagat

SEQ ID NO: 352 - DNA, 202 bp, Homo sapiens
tttatccccctttccttatatttctaacaaacatggtccatgacgcttaagaaccacagtgccatgtgaaatagtatagattaagcgtgtgaaattgatactgcagtgtaggtttgtttccaaatgcagattaggcattttattttcagacaatatttagttgtgttttccttgatgccatgctgatttttattttcctgta

SEQ ID NO: 353 - DNA, 202 bp, Homo sapiens
atgcattctaatcagttgccatttcccattctccatcccatgcaacatgcaatgctggcattttaaccaagagatagatgttagggaaaggacaggtagtagcaggctgacaccatagatcttggaacagggcagggacatacagctcagtgtttaagggcacttaaactctgagttgggagagtaagtttgggtaagaccc

SEQ ID NO: 354 - DNA, 202 bp, Homo sapiens
ggctgcagagaacagcagctatcagtggtccgaactacagggtctgtggtagattatctacagagataacaggccacaacttctcttaagtctgcatattctgtgccgcttctcccatcaacagggggagtctatttccgtacctcttgaatctgggttgatcttgagccttacactaagccagcttttgaatgatgcagaa

SEQ ID NO: 355 - DNA, 202 bp, Homo sapiens
cgtacctcttgaatctgggttgatcttgagccttacactaagccagcttttgaatgatgcagaagggatgttctgggatttccacactcaggccttaagacgatctggtagcttctactttaactcttactattctgagacaccacatgaaaaagcccaggctattcagctggagagagaggctaggtggagaggaaggccc

SEQ ID NO: 356 - DNA, 202 bp, Homo sapiens
ccacatggagatctctcaaggagaggccacaaggagaaccaaggtactccagcagccagccagcaccagggccatattagatcctccagctcagcccagcctcagcccagcccagcccagctcagccgacagctgagtgcagctgcattagtccagccaatcccatgtgaagtagagacaagctgttccactcagccctgcc

SEQ ID NO: 357 - DNA, 202 bp, Homo sapiens
tcaaggagaggccacaaggagaaccaaggtactccagcagccagccagcaccagggccatattagatcctccagctcagcccagcccagcccagcccagcctcagccgacagctgagtgcagctgcattagtccagccaatcccatgtgaagtagagacaagctgttccactcagccctgcccaaattcctgacccagagga

SEQ ID NO: 358 - DNA, 202 bp, Homo sapiens
caatgtctgaatggtcctggggaactcaggcagaattcaagattgtaacctcttggatatgctaagtgaggtttggaagagggaagtttagacagaaaagctgccagccttgtggtctttctggtttaaaatgaaaccccagaatctccccaaagtacagctttttggggagaagactggcaaaaataagagtgtaaaggct

SEQ ID NO: 359 - DNA, 202 bp, Homo sapiens
tgatttatcctcagtgactcaatgaataagtaaatacttaaatattcagcaagaatttacagaatctctattttatatgagccccaagctgaaccaattgctggaaggatagaaaagtcttcttgacaagttttcctgggcactgtggtgtttcttaatttgctgctgttgtttctgctatttctaattggttttgttatct

SEQ ID NO: 360 - DNA, 201 bp, Homo sapiens
gagaagaaaggaaaggtagaaaaaagattatctacaagccagtccctcctgctactctttaactgaaaccatgaagaatgaacccaagcttaatacatttggtatgccctgtgttgtgtatttgtgtgcatgatgttgtgggttattgtatgcatcactgttcctttgagaacttagtggaattccattaggttccaattt

SEQ ID NO: 361 - DNA, 202 bp, Homo sapiens
tatgccctgtgttgtgtatttgtgtgcatgatgttgtgggttattgtatgcatcactgttcctttgagaacttagtggaattccattaggttccaatttaatcccatcaaacaatgtcataaatcaggaacaatttaccttcctgatctaactgggaaggactttactcagattaaaaagagttaaagttccaagggaatca

SEQ ID NO: 362 - DNA, 202 bp, Homo sapiens
tgtgtatttgtgtgcatgatgttgtgggttattgtatgcatcactgttcctttgagaacttagtggaattccattaggttccaatttatcccatcaaacaagtgtcataaatcaggaacaatttaccttcctgatctaactgggaaggactttactcagattaaaaagagttaaagttccaagggaatcactaactctcctt

SEQ ID NO: 363 - DNA, 202 bp, Homo sapiens
actaactctcctttgttgggaaaagtgctcctaatttgacgtttcctcacattccagccatacctcatggtggaggaactaggagatttccgggtccatgagtgtatgatttaatgatttgtgaaaggcaagaatgttaacttagttgattactcaaaactgactgataaggtatcagacaggttaatttgctacttttcta

SEQ ID NO: 364 - DNA, 202 bp, Homo sapiens
cttagttgattactcaaaactgactgataaggtatcagacaggttaatttgctacttttctaacctctcccagagaacttgaaccaaagcctccttctccactaggtcatctttattgccctccgcatggaaggctaagaatttttgaaggaaacaagcattttattcctgtgctttcatttgctccatcattttattccaa

SEQ ID NO: 365 - DNA, 202 bp, Homo sapiens
aatttgctacttttctaacctctcccagagaacttgaaccaaagcctccttctccctaggtcatctttattgccctccgcatggaaggctaagaatttttgtaaggaaacaagcattttattcctgtgctttcatttgctccatcattttattccaaagagacagcctttatttgtgataacctcattatttcctcaacaac

SEQ ID NO: 366 - DNA, 202 bp, Homo sapiens
cattatttcctcaacaaccagtggtgatgtcacagttgaaatattttgatctcagatggaaaaagaaccctggcagatagggcctgtttacactgagaaactagagcttagtgctgagggtaaagagcaaggcattctccttttaattcaaacactaacagttatcagagtgttatctgggagacaagctcctggggattct

SEQ ID NO: 367 - DNA, 202 bp, Homo sapiens
ttttatatgcatttattacaggaaataaggctaaagtcaaaagttttaaaagtgaattataggccgggcacagtagctcatgcctgtagtcccagcacttctgggaggccaaggcgggtgaatcacctgaggtcaggagtttgacaccaacctggccaacacggtaaaaccctgtctctactaaaaatacaaaaagttagcc

SEQ ID NO: 368 - DNA, 202 bp, Homo sapiens
ggccaaggcgggtgaatcacctgaggtcaggagtttgacaccaacctggccaacacggtaaaaccctgtctctactaaaaatacaaaaagttagccaggcagtggttgtgggagcctgtaatcccagctacttgggaggctgaggcaggagaatcgcttgaacctggcaggcagaggttgcagtgagccgagatcgcaccac

SEQ ID NO: 369 - DNA, 202 bp, Homo sapiens
agttagccaggcatggttgtgggagcctgtaatcccagctacttgggaggctgaggcaggagaatcgcttgaacctggcaggcagaggttgcagtgagccagagatcgcaccactgcactccagcctgggcaataagagagaaactctgtctcaaaaaaaaaaaaaaaaaaaaagtgaatgataaattgaaatggtctcaat

SEQ ID NO: 370 - DNA, 202 bp, Homo sapiens
aaaaaaaaaaaaaaaaagtgaatgataaattgaaatggtctcaattgcatccttgagtacttggcatttaacaaaccaaaggatattttttaatgataaaagtaaccctgtgagaggacataaaaattccacgatatgattctatcatgtattagagacttaaaaaaaacactactaacaataacatataagattacattta

SEQ ID NO: 371 - DNA, 202 bp, Homo sapiens
ataagattacatttacagaattgtatacagaagatatactaataatatagacccccaatatatgtctaaacaattcaagatgatctaaacttcttttctgcttcagattttattattttaagaaacacatgcatatgcatgtttagactgttagttatgcatatattttttgaatttaattgggtgagatatttgtgacaag

SEQ ID NO: 372 - DNA, 202 bp, Homo sapiens
tctgttcagattttattattttaagaaacacatgcatatgcatgtttagactgttagttatgcatatattttttgaatttaattgggtgagatatttgtgagcaagtgaatgcaggcttcaaatgtgggttgagtgggaaataaaattcagatttaaggatgatcatctaaagctattatgtcattaacattatagcaggca

SEQ ID NO: 373 - DNA, 202 bp, Homo sapiens
aataaaattcagatttaaggatgatcatctaaagctattatgtcattaacattatagcaggcacagcaatacgttaacatcatagacctgggaaagctggagcagagttgggagattgtgatggtcctaaaaaaggccctgataatcatttagcatgaaattatatagccttcagtaaaaactttattataattctgtgcat

SEQ ID NO: 374 - DNA, 202 bp, Homo sapiens
tctttttcacctgattttctactaaatccagagtctcaaagcagtctatgtagagagcaccctctgtgaatgtatggataaaatgcatcctcccctggtcagccaggtaaaggagctactaattctccttgcctcagggtcacacatgctaggcctcaaatccccttggctttctaatcagccttggttctggattttcatt

SEQ ID NO: 375 - DNA, 202 bp, Homo sapiens
tgtagagagcaccctctgtgaatgtatggataaaatgcatcctcccctggtcgccaggtaaaggagctactaattctccttgcctcagggtcacacatgcctaggcctcaaatccccttggctttctaatcagccttggttctggattttcattcgttccttgttgaaggtgaacatgcagaatttattctacacaatgtcg

SEQ ID NO: 376 - DNA, 202 bp, Homo sapiens
ggtaaaggagctactaattctccttgcctcagggtcacacatgctaggcctcaaatccccttggctttctaatcagccttggttctggattttcattcgtctccttgttgaaggtgaacatgcagaatttattctacacaatgtcgctagtgacaaacgaagaaaagaaagggccagctagaacttaacagttccaactatc

SEQ ID NO: 377 - DNA, 202 bp, Homo sapiens
ctggattttcattcgttccttgttgaaggtgaacatgcagaatttattctacacaatgtcgctagtgacaaacgaagaaaagaaagggccagctagaactctaacagttccaactatctcaaccttcattctactgaaaaccacagaatgcctcacttctgtagagtcctctttatctaaggcaaacagtgctaagtagcac

SEQ ID NO: 378 - DNA, 202 bp, Homo sapiens
gtcgtagactcaatggacaaatatagaaccaaatgccacccttttaacagtgctctgagactggccggggaagagggtgcttatgttgaagaagcccaggagaaagctctgcatgtgaccaggaaggagagaggccagggactaggaaagagcaggcaggggccaaaagggaagtcaaatgtggtgcactgggcatgcatct

SEQ ID NO: 379 - DNA, 202 bp, Homo sapiens
aagaagcccaggaaaagctctgcatgtgaccaggaaggagagaggccagggactaggaaagagcaggcaggggccaaaagggaagtcaaatgtggtgcacctgggcatgcatctttcagccagcctggcgaaggttcaaaatgagctctgtaacctcctgggtatgtgacctgtggtgtgtacttaacctttctgatcctcg

SEQ ID NO: 380 - DNA, 202 bp, Homo sapiens
ctaggaaagagcaggcaggggccaaaagggaagtcaaatgtggtgcactgggcatgcatctttcagccagcctggcgaaggttcaaaatgagctctgtaactctcctgggtatgtgacctgtggtgtgtacttaacctttctgatcctcggctcctcgcctctaaaattatgataataagattgccttcacagtgctgtcgt

SEQ ID NO: 381 - DNA, 202 bp, Homo sapiens
ccctgtgataaaatacaaatagaccccaaaagaggtctgtgaaataaagccatttggaactaaggtggcatgtaagataatatatttaagagtgatttctagtttattaattcattcatttcaccaatatttatttctcaaccacattattccagccactgttccaatcttttttttttttttttttggagacagggtctca

SEQ ID NO: 382 - DNA, 202 bp, Homo sapiens
ttcatttcaccaatatttatttctcaaccacattattccagccactgttccaatcttttttttttttttttttggagacagggtctcactctgtcacctagtactggagtgcagtggcacgatcttggctcaccacaacctccacctcccaggctcaaacaattctcctgcctcagcctcctgagtagctgggattacaggc

SEQ ID NO: 383 - DNA, 202 bp, Homo sapiens
atatttatttctcaaccacattattccagccactgttccaatcttttttttttttttttttggagacagggtctcactctgtcacctatactggagtgcaagtggcacgatcttggctcaccacaacctccacctcccaggctcaaacaattctcctgcctcagcctcctgagtagctgggattacaggcataaaccactac

SEQ ID NO: 384 - DNA, 202 bp, Homo sapiens
tttttttggagacagggtctcactctgtcacctatactggagtgcagtggcacgatcttggctcaccacaacctccacctcccaggctcaaacaattctccgtgcctcagcctcctgagtagctgggattacaggcataaaccactaccactcagctaatttttgtattcttaatagagacggaggtttcaccatgttggcc

SEQ ID NO: 385 - DNA, 202 bp, Homo sapiens
agagacggaggtttcaccatgttggccaggctggtctcgaactcctgacgtcaaatgatccacctgtctctgcttcccaaagtgctgggattacaggcgtcgagccaccatgcccagccagttccagtcatttcagagggaaaaacaaataagccaatagtctttgccttcaaaaagcacctagtccacaagggagacagac

SEQ ID NO: 386 - DNA, 202 bp, Homo sapiens
acctgtctctgcttcccaaagtgctgggattacaggcgtcagccaccatgcccagccagttccagtcatttcagagggaaaaacaaataagccaatagtcctttgccttcaaaaagcacctagtccacaagggagacagacacatagatatataatacgataaaatataggaaataaattaagcgtactatggtaaccttaa

SEQ ID NO: 387 - DNA, 202 bp, Homo sapiens
tagatatataatacgataaaatataggaaataaattaagcgtactatggtaaccttaagacatggatggtaacctggaagcaagcagactagaactgtggagagacacggaagcctgcttgggggaggcatcaaatttgactttcgtaagatgaatagacatttgccagactgtacagcaaagacagaatgtttcatttaga

SEQ ID NO: 388 - DNA, 202 bp, Homo sapiens
tatggtaaccttaagacatggatggtaacctggaagcaagcagactagaactgtgggagacacggaagcctgcttgggggaggcatcaaatttgactttcagtaagatgaatagacatttgccagactgtacagcaaagacagaatgtttcatttagagacggcctcagcaaaattatcaaggcatgaaacagcatcatgtg

SEQ ID NO: 389 - DNA, 202 bp, Homo sapiens
gacagaatgtttcatttagagacggcctcagcaaaattatcaaggcatgaaacagcatcatgtgtgaacagcacagaaaccacgtgaatgtgtgaggcaggtttgattgtgggagatgaggctggagaattacggaggagaagaattacatatagcatgttgcaggtgtgggaaaagggggtcagaggaaaacggaggctag

SEQ ID NO: 390 - DNA, 202 bp, Homo sapiens
aattacatatagcatgttgcaggtgtgggaaaagggggtcagaggaaaacggaggctagagatgaagaagctgttggaactagttgaactgagcctggaagtaaatgggatgagatgagaggtgatcaggagcaggtgattaggtctggtcttagcaaaaggaagggcacctcatcatctgagaaggtagagaaagaggcga

SEQ ID NO: 391 - DNA, 202 bp, Homo sapiens
gtagatgaagacaaggttatagatagcagaagacagaaggttcacctaggaagcctctaatttttgtgtacttagaagcatggttatttactgagatgaggtggcgtagcatggactgatttggaggtttgaggagagggtgaaagttcagaagagtagctcaaggatctgagcatctatgaatgaatacgtcgtagagttg

SEQ ID NO: 392 - DNA, 202 bp, Homo sapiens
aagaaagaaagagagagagagaggaaaagaaagaaagaaactaactcaagaaaattgagtgtgcttctaagaaaagagttgagatggccatatagaagacctcagagaagacacaaaggcaagaggtcaagagacagtggtgacatcaaggatgacggcattgaaagaacaagcacgccatagggagggaggaagagacttg

SEQ ID NO: 393 - DNA, 202 bp, Homo sapiens
gcatactgctccagttccacataacggggcatgtcaaaacctactctcccatctgaacactcagcagtgaccaaatgggctggaggaagtgagagaaatacgcatgaaaatcctgggctgcacctcagagactaataaataattactaatgggaaagaaaacagaggaaagagaaagagcaaaggagcaggcaggacaggcg

SEQ ID NO: 394 - DNA, 202 bp, Homo sapiens
ccatgaaaatcctgggctgcacctcagagactaataaataattactaatgggaaagaaaacagaggaaagagaaagagcaaaggagcaggcaggacaggcaggaatggattctggcccatgagaggggaacagatgcaaacaaaaaaatttaacacaccaggagcagtgacccaaagacagcaaaagaaagagagtcaagct

SEQ ID NO: 395 - DNA, 202 bp, Homo sapiens
caaacaaaaaaatttaacacaccaggagcagtgacccaaagacagcaaaagaaagagagtcaagctggctggggagactcgctgttctcacatttgatacagtctgctcacaacatgtttcagtgacttattgaagctcagaattgggctgttaaatcattcagcatatgagatttgtcctgagttaaatggattacttccc

SEQ ID NO: 396 - DNA, 202 bp, Homo sapiens
ttaagtgtgagctgaggcttaggctaatgaaacctgattttagttagtgcctcatttaaatacagatgcagaaaaggaaatttctttagtgccactaagactaaggcatgcttgctgtgtagaaaacttgaactgaatctttctctctctccctctctgtctcacacacacacacacacacacacacacacacacacacaca

SEQ ID NO: 397 - DNA, 202 bp, Homo sapiens
ctctctgtctcacacacacacacacacacacacacacacacacacacacaccttccccaggagtagcccaggataaaaatgaataatacattaatttttaataacatgaagttcattcaggcttctcctgcctaacaatttctttaaaatatataactaaaaagaggattgtttcacttggtatataccattttttttctca

SEQ ID NO: 398 - DNA, 202 bp, Homo sapiens
attaatttttaaaacatgaagttcattcaggcttctcctgcctaacaatttctttaaaatatataactaaaaagaggattgtttcacttggtatataccacttttttttctcacagaagattctgatcctgagaaatatgcctgggattttggggatgactgtgacccattcagtaaacatctataaagcaactcctctaag

SEQ ID NO: 399 - DNA, 202 bp, Homo sapiens
taatttttaaaacatgaagttcattcaggcttctcctgcctaacaatttctttaaaatatataactaaaaagaggattgtttcacttggtatataccattattttttctcacagaagattctgatcctgagaaatatgcctgggattttggggatgactgtgacccattcagtaaacatctataaagcaactcctctaagtt

SEQ ID NO: 400 - DNA, 211 bp, Homo sapiens
ggggatgactgtgacccattcagtaaacatctataaagcaactcctctaagttgttctctaggttctacagggaacccacagggagataagtttctgttcccacgtcactcgtgatctagctgagggagactattcatgcaggtttgcaaattgaaataaccatgcaaagcaaccatgcctacaatgttacaaccaatgtacatcttggct

SEQ ID NO: 401 - DNA, 202 bp, Homo sapiens
tacaatgttacaaccaatgtacatcttggcttgcagaaactggtcccaccaagatatgttccatgttttgcagaaaggatgaaacttgggactgatatggctcaccaatatctgagcttgaaaacttcggtggaagttaatgtctctttgcactccttcatgtgtttgttatcacagtgctacttttaatatctttctttgt

SEQ ID NO: 402 - DNA, 202 bp, Homo sapiens
cagaaaggatgaaacttgggactgatatggccaccaatatctgagcttgaaaacttcggtggaagttaatgtctctttgcactccttcatgtgtttgttactcacagtgctacttttaatatctttctttgtgagctaagtagccatcacgttagcatactgcttttaaaatgttgtaaaacctttagagaatatattcaaa

SEQ ID NO: 403 - DNA, 202 bp, Homo sapiens
ttaatgtctctttgcactccttcatgtgtttgttatcacagtgctacttttaatatctttctttgtgagctaagtagccatcacgttagcatactgctttataaaatgttgtaaaacctttagagaatatattcaaaaggaatgattcctccctctccctcattctttccatatcatgtctgagataataaagacataaaca

SEQ ID NO: 404 - DNA, 202 bp, Homo sapiens
ggctacttgtctgtaaagccccaaggtaagctatttttaagttggtgcacccaaaaccatgatttttcaccttgatcttgactaatgtaattaatttaatagtgcatttgtaatagaaagaatataaaatatgtacagagttctttagtcttaaaatgagacatgaatctcttaggtcagtagccactgggcatgagcctgt

SEQ ID NO: 405 - DNA, 202 bp, Homo sapiens
agttctttagtcttaaaatgagacatgaatctcttaggtcagtagccactgggcatgagcctgtgccaggtgaggtgtcaactgtatacaacggtaggggcttgagagacaggaaagaccccttctggctggagtaattaaggaaagtttatggaggaaaaggtatcttgtatgagtcttgaaataagggtaggattctgtt

SEQ ID NO: 406 - DNA, 202 bp, Homo sapiens
gaagatgttgcatagggcactgcaggaagaacaagactagaaaagtaggtggaaactgattttctgagacctgaagccagactgaggaggtgaagggtgacgtagtggagtccttgggaaagtgtgaagtagagaaggacataattaaaatgtcactttcaggagattgcttcaatgtggagtgcagaatttgttggagtag

SEQ ID NO: 407 - DNA, 202 bp, Homo sapiens
tagaaaagtaggtggaaactgattttctgagacctgaagccagactgaggaggtgaagggtgagtagtggagtccttgggaaagtgtgaagtagagaaggagcataattaaaatgtcactttcaggagattgcttcaatgtggagtgcagaatttgttggagtagggagggacagtgattaggggagtctgttaggaggctt

SEQ ID NO: 408 - DNA, 202 bp, Homo sapiens
cttgccatttccagcttctcgcctctaagtcaattctcaacctactgttatttgacttcctctgctgaacacattctggacagaacttattatttccctcctcaaatctgctccttctcttatattccatatctcactcatgacaccaccaatgtcagccgaaccaaataatttcaagtcaatcctggctcctttctctgct

SEQ ID NO: 409 - DNA, 202 bp, Homo sapiens
cttctcgcctctaagtcaattctcaacctactgttatttgacttcctctgctgaacacattctggacagaacttattatttccctctcaaatctgctcctctctcttatattccatatctcactcatgacaccaccaatgtcagccgaaccaaataatttcaagtcaatcctggctcctttctctgctgaacccttcttcat

SEQ ID NO: 410 - DNA, 202 bp, Homo sapiens
catgctatctctatttagctcatcctaccattgtacaatcacattatcctagtgtgatcacagtgatcagatctctctcatccagccatggtaaaatagcagtagtggttcaacatttgaatttcaacccaaacttcctgggttcaaagccatatactcacacctttagctctatggccacggacagatttagctctttttg

SEQ ID NO: 411 - DNA, 202 bp, Homo sapiens
cctaccattgtacaatcacattatcctagtgtgatcacagtgatcagatctctctcatccagccatggtaaaatagcgtagtggttcaacatttgaatttcgaacccaaacttcctgggttcaaagccatatactcacacctttagctctatggccacggacagatttagctctttttgcttccatttctttatctgtaaga

SEQ ID NO: 412 - DNA, 202 bp, Homo sapiens
atccagccatggtaaaatagcgtagtggttcaacatttgaatttcaacccaaacttcctgggttcaaagccatatactcacacctttagctctatggccactggacagatttagctctttttgcttccatttctttatctgtaagatggtaaatcagagtaggctgttaggagttacatgagctaatatttgcctagcactt

SEQ ID NO: 413 - DNA, 202 bp, Homo sapiens
ccttccactaatgcatgatgttaaccatgcctctcttccttaactctgtaggctggaatctaagctgggtaacaaaacgtagaaacttttccatggtctgagcccctgcttcatcctcccatctcatctcctaccctcgcctcacagtttgtgtttagataatattgtgtcacatgaagttctccaagtccaccacactcaa

SEQ ID NO: 414 - DNA, 202 bp, Homo sapiens
cctccgaagagtctgcctgcttgccttgtccccatccccctctcctcagtactgcttactcattcctgaaatgccagctcaggcatcaggcatcatttccaccccaggcagccttccgtgaacgtgcaggccgggttaagtgcctcctctctccacctacaagataattttatcacggcacgaatagtatagtgctgaaatc

SEQ ID NO: 415 - DNA, 202 bp, Homo sapiens
tgccttgtccccatccccctctcctcagtactgcttactcattcctgaaatgccagctcaggcatcaggcatcatttccccccaggcagccttccgtgaactgtgcaggccgggttaagtgcctcctctctccacctacaagataattttatcacggcacgaatagtatagtgctgaaatcatctttatgatcctgctccca

SEQ ID NO: 416 - DNA, 202 bp, Homo sapiens
aatgccagctcaggcatcaggcatcatttccccccaggcagccttccgtgaacgtgcaggccgggttaagtgcctcctctctccacctacaagataatttgtatcacggcacgaatagtatagtgctgaaatcatctttatgatcctgctcccagatctgactgaaaacttcttgagggcaggagctgtgtcatattcacct

SEQ ID NO: 417 - DNA, 202 bp, Homo sapiens
gcatcatttccccccaggcagccttccgtgaacgtgcaggccgggttaagtgcctcctctctccacctacaagataattttatcacggcacgaatagtataggtgctgaaatcatctttatgatcctgctcccagatctgactgaaaacttcttgagggcaggagctgtgtcatattcacctctaaaccccacagagacaag

SEQ ID NO: 418 - DNA, 202 bp, Homo sapiens
tctttatgatcctgctcccagatctgactgaaaacttcttgagggcaggagctgtgtcatattcacctctaaaccccacagagacaagagccatgctagactatatagtaggttttcataagcatttattgaagtaaagaaaataattcaacagggttggcaattaactaaatgtggaagtagcaagacagagggagattgg

SEQ ID NO: 419 - DNA, 202 bp, Homo sapiens
aaacttcttgagggcaggagctgtgtcatattcacctctaaaccccacagagacaagagccatgctagacatatagtaggttttcataagcatttattgaaggtaaagaaaataattcaacagggttggcaattaactaaatgtggaagtagcaagacagagggagattggaagctgattccaaagtttgaagtccaagtta

SEQ ID NO: 420 - DNA, 202 bp, Homo sapiens
cagggaagtcagaaggaggaacttttttgaaaggaaaggaaaaaaaaaaccctaatgagttaatttttaaacaaactgagttggagatgccaacaaaatactgcagttagaaaatagaccctgtggtactagaaaaaggccgtggtaggtttgagggtcattgaatttgtcatagtttagtagattatgctgcctaacaaac

SEQ ID NO: 421 - DNA, 202 bp, Homo sapiens
ctcagtgggttataattgcaaaagcctattgctgctcacacagcatgtctcctgtgagtcagttgcagctctgcctggggctttgttaactctgttcagggtttacgtgtgcatctggtgcgtactgttctcatagtgaaaggtcagaataagaagggacacggagccaaccacacaagcacatttaaagtcttgctcagaa

SEQ ID NO: 422 - DNA, 202 bp, Homo sapiens
tgggttataattgcaaaagcctattgctgctcacacagcatgtctcctgtgagtcagttgcagctctgcctggggctttgttaactctgttcaggtttacagtgtgcatctggtgcgtactgttctcatagtgaaaggtcagaataagaagggacacggagccaaccacacaagcacatttaaagtcttgctcagaagtgac

SEQ ID NO: 423 - DNA, 202 bp, Homo sapiens
gttgcagctctgcctggggctttgttaactctgttcaggtttacgtgtgcatctggtgcgtactgttctcatagtgaaaggtcagaataagaagggacacaggagccaaccacacaagcacatttaaagtcttgctcagaagtgacatacataatttccactcatatcccattggccaaagcaagtcccatagccaagcctg

SEQ ID NO: 424 - DNA, 202 bp, Homo sapiens
atatcccattggccaaagcaagtcccatagccaagcctgaagtcagtggagtgaggaggcatggtaacgacaagggaataaaatattatgaacaaatatccgcactcagcacagtgaggacggagggaattgtgtgtgtagaccagatcatacacttaatttcccattgcgggggaaagagaaaccaaagaaggaagaaaga

SEQ ID NO: 425 - DNA, 202 bp, Homo sapiens
gtcagtggagtgaggaggcatggtaacgacaagggaataaaatattatgaacaaatatcgcactcagcacagtgaggacggagggaattgtgtgtgtagactcagatcatacacttaatttcccattgcgggggaaagagaaaccaaagaaggaagaaagaaaatagcataaaggtaatcagagaggtaaagaagagaacta

SEQ ID NO: 426 - DNA, 202 bp, Homo sapiens
actctcatcaaatatcaggagaattgtcacaaggggcaagagacccctccaggctctgggcttgtctagagaggaagattactgacagtagctgcatgatcgctagccaacagacttgctaagaggagaatcttttacaactgagaaagtaaaaaaatggagtggtagccttcatcaaaaatggagttgtcgccctcatgaa

SEQ ID NO: 427 - DNA, 202 bp, Homo sapiens
tgagaaagtaaaaaaatggagtggtagccttcatcaaaaatggagttgtcgccctcatgaaacaatcatctcaatgccgttggagttttcaagcagaatcatagtccaccatacaacaaacatgcggaaaagtgcatgcactggacaaaaagaccaacagatatatctatacatctatctatctatctatctatctatctat

SEQ ID NO: 428 - DNA, 202 bp, Homo sapiens
gctgcaagccaagaaatgccaaaaattgccagcaaaggggcggcacaagaagctaggaaggggaggaagaaggtttttccctgatagatttcagagggagctaaagcccttccaacaccttgattacggacttctagctccaaaactataaaagaataaatttctgggccgggtgcggtggctcacgcctgtaatcccagca

SEQ ID NO: 429 - DNA, 202 bp, Homo sapiens
ttgggaggccgaggcgggcggatcacgaggtcaggagatcgagaccatcccggctaaaacggtgaaaccccgtctctactaaaaatacaaaaaattagccagggcgtggtggcgggcgcctgtagtcccagctacttgggaggctgaggcaggagaatggcgtgaacccgggaggcggagcttgcagtgagccgagatcccg

SEQ ID NO: 430 - DNA, 202 bp, Homo sapiens
ggccgaggcgggcggatcacgaggtcaggagatcgagaccatcccggctaaaacggtgaaaccccgtctctactaaaaatacaaaaaattagccgggcgtaggtggcgggcgcctgtagtcccagctacttgggaggctgaggcaggagaatggcgtgaacccgggaggcggagcttgcagtgagccgagatcccgccactg

SEQ ID NO: 431 - DNA, 202 bp, Homo sapiens
ggcgggcggatcacgaggtcaggagatcgagaccatcccggctaaaacggtgaaaccccgtctctactaaaaatacaaaaaattagccgggcgtggtggcgtggcgcctgtagtcccagctacttgggaggctgaggcaggagaatggcgtgaacccgggaggcggagcttgcagtgagccgagatcccgccactgcactcc

SEQ ID NO: 432 - DNA, 202 bp, Homo sapiens
gggcggatcacgaggtcaggagatcgagaccatcccggctaaaacggtgaaaccccgtctctactaaaaatacaaaaaattagccgggcgtggtggcgggctgcctgtagtcccagctacttgggaggctgaggcaggagaatggcgtgaacccgggaggcggagcttgcagtgagccgagatcccgccactgcactccagc

SEQ ID NO: 433 - DNA, 202 bp, Homo sapiens
agatcgagaccatcccggctaaaacggtgaaaccccgtctctactaaaaatacaaaaaattagccgggcgtggtggcgggcgcctgtagtcccagctactctgggaggctgaggcaggagaatggcgtgaacccgggaggcggagcttgcagtgagccgagatcccgccactgcactccagcctgggcgacagagcgagact

SEQ ID NO: 434 - DNA, 202 bp, Homo sapiens
ggaaactttatattccattatttgctttttcctgagaagttcttggcacattgtgaactggagggtacaacgcaaaggacattaggctttcagtctgggaagtgctttagagacaggtgaccagtgcttcagaatgtctgcttggaagctatggtggaatgtttgtacaggtatataagagggcacagacaagcacaccagg

SEQ ID NO: 435 - DNA, 202 bp, Homo sapiens
cagaatgtctgcttggaagctatggtggaatgtttgtacaggtatataagagggcacagacaagcacaccaggatacagagagacatctggatgtgggagacaactcttatggccatttaatgcccaccatctttattttataggaaaataccagggatgtgcccaaggccactcatgccaagtatggactaggctaaattc

SEQ ID NO: 436 - DNA, 202 bp, Homo sapiens
cagttcattttcacgtacggttacatacaagacacaacaatttgccctaattgtatcccctcactttcagaggagtaatcctcaacccaaactccagtctcgctgaagatattctcatcaacagctttgttttattttaaaccaaggctttctttccactctgaaatgccaccagcaattataggaccagaaggaatatcca

SEQ ID NO: 437 - DNA, 202 bp, Homo sapiens
tacatacaagacacaacaatttgccctaattgtatcccctcactttcagaggagtaatcctcaacccaaactccagtctgctgaagatattctcatcaacaggctttgttttattttaaaccaaggctttctttccactctgaaatgccaccagcaattataggaccagaaggaatatccatttcagttcaactgttgtcat

SEQ ID NO: 438 - DNA, 202 bp, Homo sapiens
agaatacaagagtttgcaaatattttgagagtttagaggctaatcactgctgagtcataaaaaaattctcttctttgttttgatgccaacagttgtgccacttaaaataaaaaaactcattgctgctctatccttggtagcatggagactcatggatttcctttgaatttttcagctctactcaggagcccagttcaagggc

SEQ ID NO: 439 - DNA, 202 bp, Homo sapiens
gtagaaagtggcagacctgaagctgagagacaggagtgtacaacttaaagattccattctttctttctatgccatgttgttgctctcaaaatactacctcctcacttatcgattactaatagaatagaaacttatctgcactatccttacagcactattctcatagccttcctgcttataaataggcatcatcatccttttg

SEQ ID NO: 440 - DNA, 202 bp, Homo sapiens
gtacaacttaaagattccattctttctttctatgccatgttgttgctctcaaaatactacctctcacttatcgattactaatagaatagaaacttatctgctactatccttacagcactattctcatagccttcctgcttataaataggcatcatcatccttttgcaaacacagaagctgaagctgaagaagttccatttat

SEQ ID NO: 441 - DNA, 202 bp, Homo sapiens
agctgaagaagttccatttatgatagtaaaagagggtgctaacccacagagccaggtctctgggccacaaacccttccagccaggctgcctctcaatggagtcaatggaagggaagctccatgcacttaggacagaaatgtcagtagctggagttcagctagtgacctaaaacgctgatcttctctttacaacccccagatt

SEQ ID NO: 442 - DNA, 202 bp, Homo sapiens
attcatcatcagggtacagtggctcacgcctgtaatcccaacactttgggaggctgaggtgggcagatcacttgagttcaggagttcatgaccagcctggcgcaacatggtgaaaccccatctctactaaaatacaataactagccagacgtgacggtgcgtgcctgtaatcccagctaaattataaaaaattttaaaaaag

SEQ ID NO: 443 - DNA, 202 bp, Homo sapiens
tcccaacactttgggaggctgaggtgggcagatcacttgagttcaggagttcatgaccagcctggccaacatggtgaaaccccatctctactaaaatacaagtaactagccagacgtgacggtgcgtgcctgtaatcccagctaaattataaaaaattttaaaaaagaaaaattagtcatcatactatttataacaacattt

SEQ ID NO: 444 - DNA, 202 bp, Homo sapiens
gaggctgaggtgggcagatcacttgagttcaggagttcatgaccagcctggccaacatggtgaaaccccatctctactaaaatacaataactagccagacagtgacggtgcgtgcctgtaatcccagctaaattataaaaaattttaaaaaagaaaaattagtcatcatactatttataacaacatttctgcatagaattgc

SEQ ID NO: 445 - DNA, 202 bp, Homo sapiens
tttagaaatgctacctggtaagtgatcaaagatcaaagctaacactagcaataagagttattgtcataatatatcctctgatgcgctgccttgaggagaagtctagcatcacttttttggtgtttctgtcaaaaattcataacttcagtctaatcatgagtaaaaaatatcagataaatccaaacagacattcttcgaaata

SEQ ID NO: 446 - DNA, 202 bp, Homo sapiens
atccaaacagacattcttcgaaataactgagcagtactcgtcacaagtgaaaatatcatgaaagaaaaggaaagattaaaaaaactgtcacggattgggaagaaaaggtgacataacaactaaatgggatgtgggatcctagactagatcctgtaacataaagaggatgttcctggaaaaattagtacaattcacaaaaaca

SEQ ID NO: 447 - DNA, 202 bp, Homo sapiens
gtacataaactaactgaaagcctaacttagttattattttgtaataaatagctttgtctcagccaatcacaggcttccagtgattattagtccatgttcaacataaggcaagcacagaacgggagtcaatcaaagctgtctctgtacctcacttctgttttctgttcataaagtctgcctccccatgttgtgaggagagctc

SEQ ID NO: 448 - DNA, 202 bp, Homo sapiens
acttagttattattttgtaataaatagctttgtctcagccaatcacaggcttccagtgattattagtccatgttcaaataaggcaagcacagaacgggagctcaatcaaagctgtctctgtacctcacttctgttttctgttcataaagtctgcctccccatgttgtgaggagagctctctgaatgttttctggttctgagg

SEQ ID NO: 449 - DNA, 202 bp, Homo sapiens
tctctgtacctcacttctgttttctgttcataaagtctgcctccccatgttgtgaggagagctctctgaatgttttctggttctgagggctgcccaattcagagagttgttctctgctcaattaagttctgttaaacttaatttttctagtttttcttttaacaataccaatgttaatggccctgttttgatcactgtatta

SEQ ID NO: 450 - DNA, 202 bp, Homo sapiens
gtattgcctttctaggaatatttgcattttaataaaattagtttgatgccatgagagaattcagtagcagattgtattttctaaaggtgacatcaacaatagtctcccatcgtacaatcttttcagaaactttccattcctccatcaaaggtagaatctatgtaccctcctctggtatatgcaagattttgtaaatgccttg

SEQ ID NO: 451 - DNA, 202 bp, Homo sapiens
catcgtacaatcttttcagaaactttccattcctccatcaaaggtagaatctatgtaccctcctctggtatatgcaagattttgtaaatgccttggccaactagagcatggtagaagtggtggcatgtgatttccaaggataggtctttaaaatgacatacattttagcctgttctcttgtgttgctcattcttgggacata

SEQ ID NO: 452 - DNA, 202 bp, Homo sapiens
atatgcaagattttgtaaatgccttggccaatagagcatggtagaagtggtggcatgtgatttccaaggataggtctttaaaatgacatacattttagccctgttctcttgtgttgctcattcttgggacatagccaccatgctgtaataaaatctaagcggctctttaataaaaataaatagacaggcatggtggcacgca

SEQ ID NO: 453 - DNA, 202 bp, Homo sapiens
aaacaggaagatcacttgaattcaggagtttgaggctatgtgatcaaagtcactgatcatgcctctgcattgcagcagctcatggagaggtccatgtggccttctgaccatatccctagctgagctcccaggtgatagccagcacccactggccaaccatttgaatgaactgtcttgatagcaagcacatcatccagctccc

SEQ ID NO: 454 - DNA, 202 bp, Homo sapiens
agctgagctcccaggtgatagccagcacccactggccaaccatttgaatgaactgtcttgatagcaagcacatcatccagctcccagtcaagccactcagcgtaacaccacacagagaagatacggccagccccactgcacccttctccacttacaaatttgtaagcaaaatatattattgttattttcacccattaagctt

SEQ ID NO: 455 - DNA, 202 bp, Homo sapiens
tagatggggccaatagagtagggcagggtcctggtgtggattctcagggaagatggtgtctagagtcaatatcaaagttagtccccagggctggcctaccagtagtagatgaagattggggtttgaggtgccaattgatcatgctactttttccatcttggtttaaatcagttgagaacagacccaacgagaattcaagtcc

SEQ ID NO: 456 - DNA, 202 bp, Homo sapiens
attttgcctttgagaaagacagggcttgcgtttaagggcaaaggggagctgcccactccttcacatcctcatctagaactcgcccgcaggaagacctgggctttgtggagcagacactgggcagaccatgggggtcactccctggctcacaccccaggaaggagcccccaaagttttaggcctatctctaagaggccagccc

SEQ ID NO: 457 - DNA, 202 bp, Homo sapiens
acaggcatgaaccgttatgcacagccaattttatttgttattttttttgtaaagaagagggtctcactatgttgcctaggctagtctcgaacttctagtcctcaaatgatcttcccttgtcggcctcccaaagtgctgggattacaggcgtgagccactgtgcctggttgaaaaattgcatttctaacaagtcctaggtgat

SEQ ID NO: 458 - DNA, 202 bp, Homo sapiens
tataattcagaagacatacacacagcttatcttgtttaattctcacatcattcgtcattgacaaatgagggaactgtggctgatgaggaatgagggtcttagtctaagctcatacaacactaagcaaaagacctaggcttcaaatatgtcttatgatggcgacatgaagattcttttcactgaactatagctgtttcctgga

SEQ ID NO: 459 - DNA, 202 bp, Homo sapiens
atactttgaggatgatgagagctttatattacccttcctattgattcatttgacttgcacctgtgatgccaaactctccattagtttttcctaaccacttatattaactttttgaaaaaccaggaccaaagaccaaccattgagaaggactcgtgtaggtcatgatctgatgagggcaatttgctatgcctccttttttatg

SEQ ID NO: 460 - DNA, 202 bp, Homo sapiens
tcctattgattcatttgacttgcacctgtgatgccaaactctccattagtttttcctaaccacttaattaactttttgaaaaaccaggaccaaagaccaactcattgagaaggactcgtgtaggtcatgatctgatgagggcaatttgctatgcctccttttttatgttgccaaaacgaggaccagaactcttgtggacatt

SEQ ID NO: 461 - DNA, 202 bp, Homo sapiens
tcttgatgccttacttagcacaactgaacctattatcacaactgtttgtgacttcttggtgacctggactgtgaggcagctgctccggcctcatgaccccagttttatgaaccaaagttagtagcatgcctagccagcaattcctcagttcttgtgatgtgtgtgtgcatttacgtgtgtatgtgtgtgtaaatgaaggtag

SEQ ID NO: 462 - DNA, 202 bp, Homo sapiens
agttttgctcttgttgcccaggctggagtacaatggcacaatctcagctcaccacaacctccgcctcctgggttcaagcgattctcctgcctcagcctcccggaatagctgggattacaggtgtgcaccatcacgcccggctaattttgtatttttagtagagatggggtttcaccacattggccaggctagtttcgaaccc

SEQ ID NO: 463 - DNA, 202 bp, Homo sapiens
tgtagatgaattttcacaaactgaacacacttgagtaactagataaataaacagaacattaccagctccatggaagttcctattgtattcctttcttgtcagctaccctgaccaagaataacccctatttctgacttctgacactatatatgaattttagacacatatacaaatgtatcatatagtatactcttttgtatct

SEQ ID NO: 464 - DNA, 202 bp, Homo sapiens
tatgtcagccattctggtgagggtatttggtaacttgttttggttttaattcacagatgttagatatttggatattgtctttgttgaagtgccagtgtaagtccttttgcccatatttccatatctttcttagcttaaaatctccttctgtcttctctattttctttactccttggaggactattaactgctttccattaaa

SEQ ID NO: 465 - DNA, 202 bp, Homo sapiens
cttagcttaaaatctccttctgtcttctctattttctttactccttggaggactattaactgctttccattaaaaaccccaaatatccaacagcatgtagatcaaatacactgtggcatattcatacactagcacactatgcaacaataaaaaataaatgatctctgggtacccacagcatacataaaactcaaaaccatca

SEQ ID NO: 466 - DNA, 202 bp, Homo sapiens
tgaggtcgggagttcgagaccagcctgaccaacatggaaaaaccccgtctctactaaaaatacaaaattagctgggcgtggtggtgcatgcctgtaatccctagctactcgggaggctgaggcaggagaatcacttgaacccgggaggcagaagttgcagtgagccgaggtcgtgccattgcactctagcctgggcaacaag

SEQ ID NO: 467 - DNA, 202 bp, Homo sapiens
ttgtttccgctcacttacggctgcctgccagtcacccagtaagcaagccagcacacttaacaagctggcccatcaccacatttaaaatatacaacgcaaactgcaggcatttccaaaccctgtgctggggaaagaaacctccccagtgagttttcccaacattttaccttttagaagccaaactgcagtcatttttcccttt

SEQ ID NO: 468 - DNA, 202 bp, Homo sapiens
ttttccctttgactctcaagggaaatctgacagggaccacgtttcagaaaacttgtctcccatagagcaccagagctgcggtattttaatagcctctctcaggcccctcccctgggaacatattattatgtgatcattagtgatgggcaaacctcactgggtttccaggtttgagtctgtgccaaaagttcagagttcttct

SEQ ID NO: 469 - DNA, 203 bp, Homo sapiens
tctgacagggaccacgtttcagaaaacttgtctcccatagagcaccagagctgcggtattttaatagcctctctcagcccctcccctgggaacatattattatgtgatcattagtgatgggcaaacctcactgggtttccaggtttgagtctgtgccaaaagttcagagttcttcttttcttgacaactcaaaatctccccta

SEQ ID NO: 470 - DNA, 202 bp, Homo sapiens
agcccctcccctgggaacatattattatgtgatcattagtgatgggcaaacctcactgggtttccaggtttgagtctgtgccaaaagttcagagttcttccttttcttgacaactcaaaatctcccctatttttcccttttatttattcatttgtttgtttatgtgtgctttgcttggtttcttcctatcttaccacactct

SEQ ID NO: 471 - DNA, 202 bp, Homo sapiens
tatttttcccttttatttattcatttgtttgtttatgtgtgctttgcttggtttcttcctatcttaccacactctccagccttttctccactttacacacaggaagaaaggaaggagatggggcagaaagcatgagtttcttgctggtttctctccattgaggtgcactggatgaaacagaattgtaatcaggccccaatta

SEQ ID NO: 472 - DNA, 202 bp, Homo sapiens
cttggtttcttcctatcttaccacactctccagccttttctccactttacacacagaagaaaggaaggagatggggcagaaagcatgagtttcttgctggctttctctccattgaggtgcactggatgaaacagaattgtaatcaggccccaattactggcacaaccagaaaagagtgactagacacctgctatacgcccag

SEQ ID NO: 473 - DNA, 202 bp, Homo sapiens
tggccacaaggctgacactcaggcctgtgtttgtaaactggacccactagccagggattcatagggtcatggggacagtggggacacatagtctcaagccctttcccatcttccattagaggaatcccagaatacaacgtgtcccatgaccttgtagaaggctgggaatgtccaggtcagaggcaacagctggcctaggagg

SEQ ID NO: 474 - DNA, 202 bp, Homo sapiens
ttggtagacagtcttggggcagaaaaaaaaatgtgttcagaatactgtaatttatgagtattgtcattttatgattgcattgatttttattttaaaagtgagtttgcaaactgaaaattgacagaaggtagaaatgcatagaatcgataatgatatcagaaatgcacaaaatatctaaaacaaggaaggtaactagatttca

SEQ ID NO: 475 - DNA, 202 bp, Homo sapiens
gattagtaatgtctgccattgcggtgcctttatggtgagtggggaaggggatgggcaatggggtgtcagcacagacatgtatattctatttctggtcttgagcaaatgttcattcaaacaactaatatgtcaaggaaattatgtgggaactgaccaaactgaatagagacctgagcgtttccttttgagtatgtggctggat

SEQ ID NO: 476 - DNA, 202 bp, Homo sapiens
tctgcatctttctggtgccatattctttgtgcctcctagacagaagctgtggaactgatgctagaagaaagagagaaatttaaatgaagaaatgatgctgctcattatttttaatggttgatttgtttttcttgtttacctagaaaacaggtggagaatagaacaaagagatttttggcctgagccaggactgagaaaccaa

SEQ ID NO: 477 - DNA, 202 bp, Homo sapiens
ccccaccatcaatgggtagaaaaattgagtttgcttctaggaaaacaaactgcaaagttacatggcaaaaggttcacaccttttgtgaaaataggaagtgcgcaaggaattgagaaaataaaccaacttaccaaaaatattaagaggctctatttaaaacattgaatagggaagtggtatcatgaggtaaataataataata

SEQ ID NO: 478 - DNA, 202 bp, Homo sapiens
gggtagaaaaattgagtttgcttctaggaaaacaaactgcaaagttacatggcaaaaggttcacaccttttgtgaaaataggaagtgccaaggaattgagagaaataaaccaacttaccaaaaatattaagaggctctatttaaaacattgaatagggaagtggtatcatgaggtaaataataataataatagtagttagta

SEQ ID NO: 479 - DNA, 202 bp, Homo sapiens
gcattcatcagcaagactttgtggatatctatccctgtgctcttccacgaacaggaatcagttactctttgcttgtatgtttctccacttgggcagttcaaggtttcttaaagatattatctaatcaacccagctaacgttgtgccagttggggcatataattttgcaacaggatgccactgtccagtctgtgaccaaggca

SEQ ID NO: 480 - DNA, 202 bp, Homo sapiens
tgtgccagttggggcatataattttgcaacaggatgccactgtccagtctgtgaccaaggcatgtgcgtacttcaggttcaatcagctgtggtggtaaggcgccgagggggagtcatacgtaataagatttccttgacagacatggctggttgtctaccaaagcattatgctcttagaggaaagctgctgttaggctggggt

SEQ ID NO: 481 - DNA, 202 bp, Homo sapiens
taattttgcaacaggatgccactgtccagtctgtgaccaaggcatgtgcgtacttcaggttcaatcagctgtggtggtaagggccgagggggagtcatacagtaataagatttccttgacagacatggctggttgtctaccaaagcattatgctcttagaggaaagctgctgttaggctggggtgtcctttcctacatttcc

SEQ ID NO: 482 - DNA, 202 bp, Homo sapiens
tggcatagtagtcattgggattagggtgtggaatgaagcaaaacataaaaggctaatcagcagagcaaagacttcagctgcagaggtctgaacatgccccgtctgggagggcctccctgatgccacactcagctgagaaggttgaattcaataggaagtcaactgtaactgccaaggagtgtggccttcataagggatgagc

SEQ ID NO: 483 - DNA, 202 bp, Homo sapiens
ataaaaggctaatcagcagagcaaagacttcagctgcagaggtctgaacatgccccgctgggagggcctccctgatgccacactcagctgagaaggttgaagttcaataggaagtcaactgtaactgccaaggagtgtggccttcataagggatgagctccaaatccacacggttgtccacatttccactgtcactcatcag

SEQ ID NO: 484 - DNA, 202 bp, Homo sapiens
ccctgatgccacactcagctgagaaggttgaattcaataggaagtcaactgtaactgccaaggagtgtggccttcataagggatgagctccaaatccacactggttgtccacatttccactgtcactcatcagggagagctttcacttaacacaaggatgtgaggatttcctttgtaaatcggttgttcctctggatatggt

SEQ ID NO: 485 - DNA, 202 bp, Homo sapiens
cctgatgccacactcagctgagaaggttgaattcaataggaagtcaactgtaactgccaaggagtgtggccttcataagggatgagctccaaatccacacaggttgtccacatttccactgtcactcatcagggagagctttcacttaacacaaggatgtgaggatttcctttgtaaatcggttgttcctctggatatggtt

SEQ ID NO: 486 - DNA, 202 bp, Homo sapiens
gggatgagctccaaatccacacggttgtccacatttccactgtcactcatcagggagagctttcacttaacacaaggatgtgaggatttcctttgtaaatctggttgttcctctggatatggtttctgaaatatctgtcttaaggtctaagctgctctctctttccatgagaagctctgctcacccaactcaaccagtttta

SEQ ID NO: 487 - DNA, 202 bp, Homo sapiens
acggttctgtcttcccttgcctggatctaagctgcttgcctctcattaacagagtccaacctcagcctgtgtgttgccaccacccactttcctagttcctctccctttcctctgacagacacacctttatactgtagggctgacttccttttggaccccaagaatctgctagctagttgctcccagccatgggaataaaagc

SEQ ID NO: 488 - DNA, 202 bp, Homo sapiens
cttgtgcaatcttatctgaaggcactgatacatttttagtaacccaccatgaacagcatcagagttgggataactggaaagatggagaaatctttctgcaagccccagcccccctttccatttaacaagagcaactagaccctgatgagattggagagaaatccattcttgcctggattgtcacctacacatatcttaaagt

SEQ ID NO: 489 - DNA, 202 bp, Homo sapiens
ctttccatttaacaagagcaactagaccctgatgagattggagagaaatccattcttgcctggattgtcacctacacatatcttaaagttttaaccattcagtgtacatatcatctaccgatgggtaattgcacttattgtaagtacagcttggtgaattgtcagaaattgaacacccctgtgtaacaagcacagggaacat

SEQ ID NO: 490 - DNA, 202 bp, Homo sapiens
agaaatccattcttgcctggattgtcacctacacatatcttaaagttttaaccattcgtgtacatatcatctaccgatgggtaattgcacttattgtaagctacagcttggtgaattgtcagaaattgaacacccctgtgtaacaagcacagggaacatcaattgcatccctcgaaacccctctcatgcccacttctagtca

SEQ ID NO: 491 - DNA, 202 bp, Homo sapiens
cttgcctggattgtcacctacacatatcttaaagttttaaccattcgtgtacatatcatctaccgatgggtaattgcacttattgtaagtacagcttggtagaattgtcagaaattgaacacccctgtgtaacaagcacagggaacatcaattgcatccctcgaaacccctctcatgcccacttctagtcattacctcccca

SEQ ID NO: 492 - DNA, 187 bp, Homo sapiens
tttttgtgtgtagttgtaggtagttcattctctttgctattaatattccattatacaccaaaaaattgtatatccattctgctgttggtggacattagggctaacttccagttggactactatgaatagtgctgttatgaacattttggtacatatcttttggtaaacacgtgtacacatctttgct

SEQ ID NO: 493 - DNA, 202 bp, Homo sapiens
gctggatttatggtatgcatgtgaattattttagtaaaacttgccagataattttccaaagtggccataaccaatgtatactcctgcaagcaatgtctgacgagttccagttgctccacatccttgccagtacttcatattttcagtgtctatttttgctcttctggagggcacgtggtgacaattgcattgtaattttact

SEQ ID NO: 494 - DNA, 202 bp, Homo sapiens
ctggagggcacgtggtgacaattgcattgtaattttactttgcacacctcacacacattagttttcatttctctgctttagttaatttccaatcatccccctgacctaacaggagccaatctgaccatcaagtcatccctttacaaagactctatctttactgagattctcgtaacacctcctccacctacctacatggact

SEQ ID NO: 495 - DNA, 202 bp, Homo sapiens
gagctcttgttgagcttagatatggagccactccatttatactcaattagctgctgggtagtgtccatgggctacacgtttttcctgtgttgttctgtctctcccaactggagataggtttccttggctctgcagagtccagctctgcaggccctgttcacatacacaaacccactgggctaaattcagatgggcataggct

SEQ ID NO: 496 - DNA, 202 bp, Homo sapiens
agtccagctctgcaggccctgttcacatacacaaacccactgggctaaattcagatgggcataggctatgcccctattggactggactttgtatttttcactccaatcattctgatgcccgatatctttagcctagatgcttgatattgattaaaaccactcaagaagttaagtagtgtgggaagtcaagtaatgcggaaaa

SEQ ID NO: 497 - DNA, 202 bp, Homo sapiens
ttatatcatttccatcttgctatctgcttggcccactatgacaaggagataccttaagtcctgctcatgcaagtggcatccttctctgctcttaagtatgacgattaagattcagacatctctgcttttaggagggtgtttggagaagtaaatgaggggagagatttgcataagaaatttcctgctcttccctggaggatgt

SEQ ID NO: 498 - DNA, 202 bp, Homo sapiens
aaatgacttaaggcttggaagggtggcctctatttttcttctttttaaacattgctgtgctgagccaatgtgggttggtgtatccaaaaccaatttggcaaggtcattagtttgtttcctctgatagtgctacactgcaggcaggattacaaaacacattccagctggctggactttctgatatctcgaaatggttccatgg

SEQ ID NO: 499 - DNA, 202 bp, Homo sapiens
attaattcttcatgcatattttttataaagtaattttcatatagatgcttgttctttgccctagtagaagaagaatgaatggaaatgtgagagtgagagactatgggaaatacatttaataacttttccaaggctctgtcatttaattttgtactaagagaaaggtgttattatcttcattttaagaaatgaaaaaaaaggc

SEQ ID NO: 500 - DNA, 202 bp, Homo sapiens
ttgtactaagagaaaggtgttattatcttcattttaagaaatgaaaaaaaaggctaaaagggatttcatatattattcaaggtgacatagctctgtgaagcttggaattagattcttaggtttgtctagatctcttccttttctcctcttctcttccacatagaatccatgcaacagttctactctccccctccacctagaa

SEQ ID NO: 501 - DNA, 202 bp, Homo sapiens
aggtatgttgagacacccaggaactaccaacagcaagaaactgtgacctccccaaggtctaaaggcacaaggaaaaggagcaataatgccaagaatgacaagccccatgggagttggagcatagataagggatcataatgactctcacacatgagacccagcaggaagagaagagggggagccatactgtgacctctctatc

SEQ ID NO: 502 - DNA, 202 bp, Homo sapiens
atctgtagatatcgcctcatgggacacaaaacaggatagtgcaaagagagaggactggggagaaggaagtacgaatggagaacaatcaacatttgaatttctcaatctttcattcaatccattgaatccacttttggcatctttttcttgtagaagctccagactaagaaaccacgcaaaaatttaacttgaatttaaattc

SEQ ID NO: 503 - DNA, 202 bp, Homo sapiens
ggcatctttttcttgtagaagctccagactaagaaaccacgcaaaaatttaacttgaatttaaattccaggaaatgggtttggtccttgagattaattatgtctttagattttctatgatgtatttacaaaatcctagacctgggatttgcagagcgcctattatcctgcactttgaggatcgcccataccccaggaagaaa

SEQ ID NO: 504 - DNA, 202 bp, Homo sapiens
aaaccacgcaaaaatttaacttgaatttaaattccaggaaatgggtttggtccttgagattaattattctttagattttctatgatgtatttacaaaatccttagacctgggatttgcagagcgcctattatcctgcactttgaggatcgcccataccccaggaagaaaggtcctgtgttaccgactgattcatgttgttcc

SEQ ID NO: 505 - DNA, 202 bp, Homo sapiens
atatattaaagacttcagagttttttttcagagaatacaatgttttcatcttttttttttaagaggcattttatagctcagggaaagtcacttaaaaaatactcattctactgagataccacatgccccatttgttcagaagtcttaactgacaggaactgctttaccactcacctgtgtctgtctaaacctcccagattct

SEQ ID NO: 506 - DNA, 202 bp, Homo sapiens
gatgtccatattgacatggctgtctacagaacaaggacagaagtaatgacatcccccacaagtgctcatgtttataccacagattgatttctgtccaaccacattttcattcacttttctattataaattagtctaactccggtaatgtacatgagttaagtaaaacaggtataagaaagcatactttggcacacatagatg

SEQ ID NO: 507 - DNA, 202 bp, Homo sapiens
ggacagaagtaatgacatcccccacaagtgctcatgtttataccacagattgatttctgtccaacccattttcattcacttttctattataaattagtctagactccggtaatgtacatgagttaagtaaaacaggtataagaaagcatactttggcacacatagatgctgtaggcctatatatgtttcttacatttaatag

SEQ ID NO: 508 - DNA, 202 bp, Homo sapiens
aggcctatatatgtttcttacatttaatagacatgttggtgcaagacatattttactgttctcttattctatattaagaaaaaaaatattgtgagtgtgactgacaaatggcaacagaggccggtatttgggggcatgggaaaccgggcagcacatatgccatctagagggatgcctgactgctttggtccagtgcactgat

SEQ ID NO: 509 - DNA, 202 bp, Homo sapiens
actggccaagagttaggagctgctaaactcagtgagacccaaaggcaaggcatctgggcagtttgacatggagtaaggagagggaatgtttgctctgattagatttccttctacaataataaaaatacagaaattgtggaatgttctagaagccaccaagaaagtactcaactgctttcagggcagtcctgggtcctgatca

SEQ ID NO: 510 - DNA, 202 bp, Homo sapiens
caagaaagtactcaactgctttcagggcagtcctgggtcctgatcaccttcttccctactcctcagagtcacctcttccccatgcacagacactcttacactacacatacaaaaacacacacacacacactggttacctaaggatgcctttccttgacgtccagtctgtgtttagacaacttagcttcctgcttgggcatta

SEQ ID NO: 511 - DNA, 202 bp, Homo sapiens
agggatcagcatagagaattaagtatgccagaacagcggttcccaatctttttggcaccagggactggtttcatggaagacaatttctccgtggacttggaggcagggggatggttttgagatgaaactgttccacctcagatcaccaggcattagcttctcataaggagcacgcaaactagatcccttgcacgcacagttc

SEQ ID NO: 512 - DNA, 202 bp, Homo sapiens
cagaccagtaccagtggcattaggtacaggactgtggtgggtgttggggaccagtgtgacagaagatatccttcagtactttaaggcatttcagagcagccttagtggctcctgctcctaaaagggatacttcacagtgttgtggtatctgaacgccttaaggaaacaagacaattctgaagttaggcccagagcaagggaa

SEQ ID NO: 513 - DNA, 202 bp, Homo sapiens
ctctagcttatgacctcatcaccatcctctacttcctctttctttgcctctgcactgtttgactggggttgccataattctgacatggcctgctggctggagttaagatcttctggctttctgagatggtctgcatcctgtttgctacatctgtatccctaagctcctcatgtcaatgtttccaaccaaaatgtattaactt

SEQ ID NO: 514 - DNA, 202 bp, Homo sapiens
tgcactgtttgactggggttgccataattctgacatggcctgctggctggattaagatcttctggctttctgagatggtctgcatcctgtttgctacatcctgtatccctaagctcctcatgtcaatgtttccaaccaaaatgtattaactttgaacccttggctcctgcactctactttgctaaaaggatgctggccttcc

SEQ ID NO: 515 - DNA, 202 bp, Homo sapiens
gatcttctggctttctgagatggtctgcatcctgtttgctacatctgtatccctaagctcctcatgtcaatgtttccaaccaaaatgtattaactttgaactccttggctcctgcactctactttgctaaaaggatgctggccttccttgactttgcatggagccaatttccctccccctccaagagcatctacctcatgga

SEQ ID NO: 516 - DNA, 202 bp, Homo sapiens
ctgctgaggttcagatccctcaggacagcatttcctgatggagtcagtcactcaattctgtatccaggcactcacattgatgacacgcaagcaaataaaactggtttatgaggaaagattgctaccgagtcttggtagtcctgagggtcaataaaaatgttacttgaggaaaagaatcatcagcgtaaagattaattatctg

SEQ ID NO: 517 - DNA, 202 bp, Homo sapiens
aaatggtttatgaggaaagattgctaccgagtcttggtagtcctgagggtcaataaaaatgttacttgaggaaaagaatcatcagcgtaaagattaattagtctgttaaatcatggtggtgagagtagctttttctattgggtgatgaatttttatgcgtgttctgacatgcatgagtttgacatattacacaattggtacg

SEQ ID NO: 518 - DNA, 202 bp, Homo sapiens
atctgttaaatcatggtggtgagagtagctttttctattgggtgatgaatttttatgcgtgttctgacatgcatgagtttgacatattacacaattggtactgacaaaaacagagaatgcacagtgaggagcccatatgaggtgactgtaagaggatctgactgcatctgagaggcaatgcactctcttgagcaactcaact

SEQ ID NO: 519 - DNA, 202 bp, Homo sapiens
acaaaaacagagaatgcacagtgaggagcccatatgaggtgactgtaagaggatctgactgcatctgagaggcaatgcactctcttgagcaactcaactcctgatgtccactaaatggaattactcctaagtgtatcacaaccatggcccagttatttaataagaaaatcatacttaatatgaatgtttaacatcttcaata

SEQ ID NO: 520 - DNA, 202 bp, Homo sapiens
gcaactcaactccgatgtccactaaatggaattactcctaagtgtatcacaaccatggcccagttatttaataagaaaatcatacttaatatgaatgtttagacatcttcaatataggcaaatgttttgtttataaaagtcaggtcaataaaacaacactactcctttctattttttatcataaataataaagaattgcttg

SEQ ID NO: 521 - DNA, 202 bp, Homo sapiens
caggaaaactgaaattactctaggtgtatcaaacagaaggaatttaatagagggacttgacagaaatatggaggagcaaggaacccatagaagggatggtgtaggcaacccacatgttaatgacagcaattaacctctaaggttagcaggagtaaatggtgttaccagggccagaatttggaagctacggccatggtgaggt

SEQ ID NO: 522 - DNA, 202 bp, Homo sapiens
atgtgcatgttttattgaactgatactgccacagtgtctattatttgcctacattattctaaggacttgataaatattatcacattttactttttcaagcagatggaaggtacagatatgaggacaggtcatgtgcactggtctatcatgtctcgtcattttgcaaatgaggaaactggaacacagagacattaagtaatag

SEQ ID NO: 523 - DNA, 202 bp, Homo sapiens
tcgtcattttgcaaatgaggaaactggaacacagagacattaagtaatagccctgcagttaaaaagttagcagttggtagagccaggatttgaacccaggcggttctacttctagagtctgtgttcttaaccactatgctatgctattgtcttcgatatggcaacctcaggttagtcagacctattgcatgaaggctcacag

SEQ ID NO: 524 - DNA, 202 bp, Homo sapiens
tttgcaaatgaggaaactggaacacagagacattaagtaatagccctgcagttaaaaagttagcagttggtagagccaggatttgaacccaggggttctacgttctagagtctgtgttcttaaccactatgctatgctattgtcttcgatatggcaacctcaggttagtcagacctattgcatgaaggctcacagctctact

SEQ ID NO: 525 - DNA, 202 bp, Homo sapiens
gaaatggaaggaaatagagtctttcctttaggcagttctctgttccaactggtattcttctaacgcctcccttaactctgacaaccttctaaatggcatcaggacaagacttccacggactcaagtttcattgatgtagttgtacccaaacagaaaatgttctgttgcctatattcaaaacacattgctaagccaattttct

SEQ ID NO: 526 - DNA, 202 bp, Homo sapiens
aggcagttctctgttccaactggtattcttctaacgcctcccttaactctgacaaccttctaaatggcatcagacaagacttccacggactcaagtttcacttgatgtagttgtacccaaacagaaaatgttctgttgcctatattcaaaacacattgctaagccaattttctctaaattctcctgaatttcctcccctggg

SEQ ID NO: 527 - DNA, 202 bp, Homo sapiens
cacggactcaagtttcattgatgtagttgtacccaaacagaaaatgttctgttgcctatattcaaaacacattgctaagccaattttctctaaattctccctgaatttcctcccctgggaggattttacaactgccaagtttgaatattaaacctttaggtacttttgagtaatactcagcagagagaaaagaaaaaaaaat

SEQ ID NO: 528 - DNA, 202 bp, Homo sapiens
aaagtgtgtgtgtgtgtgtgtgtgtgtgtgtgtgattctcagagaaaaatgtaggtatttctgttttaacttaactgtaaactactgaactaatttagctctaatcttttgaatacaagttcaatttatgaggccagtgactatcaaaaaatttaagctgaaaaataatgggaaaaccactcaactcaccaaaattctgttt

SEQ ID NO: 529 - DNA, 202 bp, Homo sapiens
ttcaatttatgaggccagtgactatcaaaaaatttaagctgaaaaataatgggaaaaccactcaactcaccaaaattctgtttttcattaaaatctgagtgttgtgacaaatgtggctttgtaaaaaatagctctggtgaatgctactattaatgtacttgatgacatttctttatgatgttttcccaagggagcaatttga

SEQ ID NO: 530 - DNA, 202 bp, Homo sapiens
cactcaactcaccaaaattctgtttttcattaaaatctgagtttgtgacaaatgtggctttgtaaaaaatagctctggtgaatgctactattaatgtactctgatgacatttctttatgatgttttcccaagggagcaatttgattggtatagagaaaaatttcaaaagaaggtttccttctgtgtcatgatctaagacacg

SEQ ID NO: 531 - DNA, 202 bp, Homo sapiens
tgacaaatgtggctttgtaaaaaatagctctggtgaatgctactattaatgtacttgatgacatttctttatgatgttttcccaagggagcaatttgattaggtatagagaaaaatttcaaaagaaggtttccttctgtgtcatgatctaagacacgaatttcacgtgcagacaaacaaatattggagtgatcaggctggtt

SEQ ID NO: 532 - DNA, 202 bp, Homo sapiens
ttcaaaagaaggtttccttctgtgtcatgatctaagacacgaatttcacgtgcagacaaacaaatattggagtgatcaggctggttggtctgtcgggtctcttatagccaaggttaattctaattacatgactggctgcaaatttgccctttcctctcccaccaatgcagaagcagacaaaagatggctattggaacacaac

SEQ ID NO: 533 - DNA, 202 bp, Homo sapiens
ttcacgtgcagacaaacaaatattggagtgatcaggctggttggtctgtcgggtctttatagccaaggttaattctaattacatgactggctgcaaatttgtccctttcctctcccaccaatgcagaagcagacaaaagatggctattggaacacaacagttcacagtaacattctctgccataacctctaaatgtgcagac

SEQ ID NO: 534 - DNA, 202 bp, Homo sapiens
aaggatgggccagcatgaaatcaaatcacccaattagttcagcaagaacttgaataaacccagtcatcaggaatatcctacatggaatatctgaatgatgagacggagttgaaggagttgacttaattagaaagctttttcagccttcctagggcaacgaaacttcttttaaagtctacatgtgcaataaagtaagcttaat

SEQ ID NO: 535 - DNA, 202 bp, Homo sapiens
ggatgggccagcatgaaatcaaatcacccaattagttcagcaagaacttgaataaacccagtcatcaggaatatcctacatggaatatctgaatgatggactggagttgaaggagttgacttaattagaaagctttttcagccttcctagggcaacgaaacttcttttaaagtctacatgtgcaataaagtaagcttaatgt

SEQ ID NO: 536 - DNA, 202 bp, Homo sapiens
tcaaatcacccaattagttcagcaagaacttgaataaacccagtcatcaggaatatcctacatggaatatctgaatgatggacggagttgaaggagttgactttaattagaaagctttttcagccttcctagggcaacgaaacttcttttaaagtctacatgtgcaataaagtaagcttaatgtagcttggacttaagctta

SEQ ID NO: 537 - DNA, 202 bp, Homo sapiens
cttctgggaccggaccaccaagatgtgattcccatttctactccttggagctggatggctttggataaattctgtaacccccctgttccttttatctttccttctataaaataggaataataaacatgtctactttacagtattgtgaagaggaccaaatgaattaatttttgcttagagtaatcagcacagtatgtgctta

SEQ ID NO: 538 - DNA, 202 bp, Homo sapiens
tactccttggagctggatggctttggataaattctgtaacccccctgttccttttatctttcttctataaaataggaataataaacatgtctactttacaagtattgtgaagaggaccaaatgaattaatttttgcttagagtaatcagcacagtatgtgcttaataaacgtggttatcatgtatgctataatcggctttgg

SEQ ID NO: 539 - DNA, 202 bp, Homo sapiens
cttctataaaataggaataataaacatgtctactttacagtattgtgaagaggaccaaatgaattaatttttgcttagagtaatcagcacagtatgtgctctaataaacgtggttatcatgtatgctataatcggctttggctattcttaacaattcatcaacataatttctctagctaccacacgtgagcagtcctacccc

SEQ ID NO: 540 - DNA, 202 bp, Homo sapiens
taggaataataaacatgtctactttacagtattgtgaagaggaccaaatgaattaatttttgcttagagtaatcagcacagtatgtgcttaataaacgtgagttatcatgtatgctataatcggctttggctattcttaacaattcatcaacataatttctctagctaccacacgtgagcagtcctacccccagtataatca

SEQ ID NO: 541 - DNA, 202 bp, Homo sapiens
cagcacagtatgtgcttaataaacgtggttatcatgtatgctataatcggctttggctattcttaacaattcatcaacataatttctctagctaccacacagtgagcagtcctacccccagtataatcatctggtggccttggggaagtcacttattgccagtgaactttgtgggattaaaaggctattctcgcatggtgac

SEQ ID NO: 542 - DNA, 202 bp, Homo sapiens
gctaccacacgtgagcagtcctacccccagtataatcatctggtggccttggggaagtcacttattgccagtgaactttgtgggattaaaaggctattctcggcatggtgacctctgcttttaaatcaaagtagctaaagcacatggaatgctgcctggtaaataaatagtagagacgcacagtctgaagcagaatggtgga

SEQ ID NO: 543 - DNA, 202 bp, Homo sapiens
aaaaatataagaaaaaagggagagagagaaaaaaataccactagggttcaaataaaggaaagggcccactactcaaatcaacatctttaaaaagaggcaaaggttggagacattagaggctctggcgtcttccccaccacctcctaaatgcaccccttgcctctattctacccctgtaacggagattagagattgtgagtga

SEQ ID NO: 544 - DNA, 202 bp, Homo sapiens
agagaaaaaaataccactagggttcaaataaaggaaagggcccactactcaaatcaacatctttaaaaagaggcaaggttggagacattagaggctctggctgtcttccccaccacctcctaaatgcaccccttgcctctattctacccctgtaacggagattagagattgtgagtgaatgcctatcttctacgggaagaaa

SEQ ID NO: 545 - DNA, 202 bp, Homo sapiens
tgcaccccttgcctctattctacccctgtaacggagattagagattgtgagtgaatgcctatcttctacgggaagaaatgtgcacttggagggtatgaggagcaatctggtgactatatgaagtatcttcattataaatcctaacaaacggaagagagaagattcgaatatataatacaagaaaatgttacctgaatgaaag

SEQ ID NO: 546 - DNA, 202 bp, Homo sapiens
agaacaaatgaatacatgacagaaaacacagatatatcttacaaaaattgcaacttcaaagatcaagataattttcttaggaatacaagtagaaaaagaaagactgagtatgaggatgaaaattaaaactggccatggacttagctacggtaacatctatcaccagtggtaatatagcaatgtcttcaagttatttgggatg

SEQ ID NO: 547 - DNA, 202 bp, Homo sapiens
ttgcaacttcaaagatcaagataattttcttaggaatacaagtagaaaaagaaaactgagtatgaggatgaaaattaaaactggccatggacttagctacaggtaacatctatcaccagtggtaatatagcaatgtcttcaagttatttgggatggtggtgggtaggtaaggtaggggttcatgtaaaaattctaaactaag

SEQ ID NO: 548 - DNA, 202 bp, Homo sapiens
aatcccagcactttgggaggctgatgcaggggatgcttgagcccaggagtttgagaccatcctgggcaacatggtgaaaccccgtctttacaaaaaatttataaaaactagccaggcatggtgatgcatgcctgtagtcatagctacccaggaggctgaggagggaggatcacttgagcccaaggggttgaggctgcagtga

SEQ ID NO: 549 - DNA, 202 bp, Homo sapiens
acttttgtatggagaatgagtagattatatctaaagtagacaaatcatgaaatagagcttcctcttatccatgtgttcaactagtgtttttgaagaccacctatgtgccaggcactgttttaagtggtatggatgtaactatggtaattcttcatttaatatcattggtaggttcttagaaatggtgactttaagccaaatg

SEQ ID NO: 550 - DNA, 202 bp, Homo sapiens
agctaaagatgagtacatttaattacatcaacatttgaattttctgtacaaaaaatatataataagatcataatacagtcaatagatggaaaaatatttactaacacatttctgagacaggacacatttctttaatagataaaattcctgtaaaaataattttaaaaagtaggtgtgataatagtaatacaatttttttttt

SEQ ID NO: 551 - DNA, 202 bp, Homo sapiens
ggtgggtggatcacaaggtcaggagatcgagaccatcttggctaacacggtgaaaccccgtctctactaaaaattcaaaaaaattagccgaatgtggtggctgggcgcctgtagtcccagctgctcgggaggctgaggcaggagaatggcgtgaacccgggaggcggagcttgcagtgagccgagactgcgtcactgcactc

SEQ ID NO: 552 - DNA, 202 bp, Homo sapiens
ccgtctctactaaaaattcaaaaaaattagccgaatgtggtggcgggcgcctgtagtcccagctgctcgggaggctgaggcaggagaatggcgtgaacccagggaggcggagcttgcagtgagccgagactgcgtcactgcactccagcctggcgacaaaaggagactccgtctcaaaaaaaaaaagaaaaaaaagaaggca

SEQ ID NO: 553 - DNA, 202 bp, Homo sapiens
gagactccgtctcaaaaaaaaaaagaaaaaaaagaaggcagaattgttaccttaccgttttactaacatttgatggtgtccaaagctgatgtgaattccacttgaggatagagtaaatcagcacatacttttgtgaaatgcgattcatgtacaatctttaaatttttttttaatgcacattttctgacccagaaaatctatt

SEQ ID NO: 554 - DNA, 202 bp, Homo sapiens
ttgtattttagtagagacggggtttcaccatgttggctaggatggtctcaatctcctgacctcgtgatccgcttgcctcggcctcccaaagtgctgggatgtacaggcatgagctaccacgcctggccaagatatatttatttaaaaaaaatcaaaaccctaggaagatgtggatattactgactgcgatgtcagacctttg

SEQ ID NO: 555 - DNA, 202 bp, Homo sapiens
tatacacaaatatacacacatgtacatctgtgcatttgccttgattctcctgtttaaatatgcatgttagccctgtttacctttagaaacaaagtcttttagttagtatttattattttattttaattttatatattttggcattttaaaaaatttttgacatgtgtatatcattattttttaagttaacagaggtcatctg

SEQ ID NO: 556 - DNA, 202 bp, Homo sapiens
ccaagtgagaccctgtcttaaaaaaaaaactttaaaatattatttaaaataaaatatgatgtcattattgtttaatttttaatagtttataaacttctcaacgttaaaatgagagagaattccctaaaatattcagctcttttgtaaactaggagcaacatatttggtttactatgtgagacatgtcttgggggctaaagaa

SEQ ID NO: 557 - DNA, 202 bp, Homo sapiens
tttctcccattatataggttgccttttcattttgttgatcctttttcttctgagcagaagcttagtggtttgatgtagtcctacttgtttatttttgcttcttgttgcctatgctatcaaaaaaattattgccaagaccaatgtcaatgagctttcccccctgtgttttcctctaggagtttcatggtttcagaccttacat

SEQ ID NO: 558 - DNA, 202 bp, Homo sapiens
taggttgccttttcattttgttgatcctttttcttctgagcagaagcttagtggtttgatgtagtcctacttgtttatttttgcttttgttgcctatgctattcaaaaaaattattgccaagaccaatgtcaatgagctttcccccctgtgttttcctctaggagtttcatggtttcagaccttacatttaagtcctaatct

SEQ ID NO: 559 - DNA, 202 bp, Homo sapiens
tatggtatatggtataagacaaggatccagttttattcttttgcctgtgtatgtttggtttcccaacaccatttattgatgataatttcctttccccattagtttattcttggtgcctttgtcaaagatcagttaactatatgtgtgtggatttatttctgtgctctttattctgtttcattggtctctgtgtctgttttta

SEQ ID NO: 560 - DNA, 202 bp, Homo sapiens
ttttgattaccatagctttgcaatatagtttgaagtaagagactgtgctgctcccagctgtgttcttttttctcaaaattgctttggctatccaggtcttcttgttgttccatacaaatgtcaagattttttttctatttctttgaaaaatgacattggaattttgatagattacattgaatatgtacatggctttaggtag

SEQ ID NO: 561 - DNA, 202 bp, Homo sapiens
tggaaacaaactaaacacccatccagggataagtgggtaaagaaattatggtacatataaacaatgaactattactcagcctttaaaaagaagaagatcagtgtcatttgtgacaacatggatgaaactggaggatgttatgctaagtgaaataagctagacacataaagacatatactgtatgatctcacttatattgtgg

SEQ ID NO: 562 - DNA, 202 bp, Homo sapiens
taaaatacaagatgaacaagttttggggatctcatgcacagcaggaatggtgatgggtatgttaatatgacagtggtaatcactgtaaaatgtatatgtactatcaggtcatcatggtgtacaccttaaatatatttaaactttgtcaattaaatactaaatatataaaatataagtaaatattttttaaaacaagaaaaat

SEQ ID NO: 563 - DNA, 202 bp, Homo sapiens
taaaacaagaaaaatcccagatatgcctggaggctttcctattagggtagacttggtggggagaccatagtgctcagaaaacagtgtggagcacagcaagagaaagtaactcagaatacaattctgaaagtgttcattaaagcacactcaatgtagaattaattaggtgttcattttatggttaatccacctcactcaagtt

SEQ ID NO: 564 - DNA, 202 bp, Homo sapiens
ttgtaaaccccacatgttgagggggagagctggtgggaggtgattggatcatggggacagtttcacccatgctgttcttgtgatagtgagtgagttctcactgatatctggttctttgataaatgtgtggcgcttccccccacaacctctcctgctgccttgtgaagaatgtgcttgcttctcctttgcgctccaccacgac

SEQ ID NO: 565 - DNA, 202 bp, Homo sapiens
cactgatgtaggacatgaaacaagagtcaattgatctaaaagttgcttcctatctaaaagtgcttcatttgtttattgctgaaaatgaaagaggacatgtagggaatgcaattcagacatctcctggtagctccataatgaaaatcacttaaaatatatgaccgaggtaaattttggcaaatacatctatacttaaaatagc

SEQ ID NO: 566 - DNA, 202 bp, Homo sapiens
tgtctgattctatgtttggcactttgggacactgtatttgctctctaactaaagtgaggtaaatccaaatttactctgaactagataagaacactcaaatagatcagagatcttttctccctataatgctttcatgagaaatgaacaaacaagtagatttgatttctctttttctctccctccctcactccctccttccctc

SEQ ID NO: 567 - DNA, 202 bp, Homo sapiens
ccttccctccctgtcttactccttccttccttgatttcttccttccttccttctttgcaacaaccactccatagtgtctcaaatggtagaacaaatataactacaatttctatatttttcctgattaacagctggtacaaagataatacttggccataaacatgtctctgaggttttcattatagaaagctaaccaaactta

SEQ ID NO: 568 - DNA, 202 bp, Homo sapiens
aataaactaaattgtattcagggatattttattataattgtgtatgaggtacatataagtaaatcataaaatttaatttgcatttcacctttaagttgttagtgtatttcatcagcattcattcctcactatattaaagtaactaacttccctggagctccatcttttctgcctacttagtagtttacttgtgtaataagac

SEQ ID NO: 569 - DNA, 202 bp, Homo sapiens
attataattgtgtatgaggtacatataagtaaatcataaaatttaatttgcatttcacctttaagttgttgtgtatttcatcagcattcattcctcactactattaaagtaactaacttccctggagctccatcttttctgcctacttagtagtttacttgtgtaataagacattgaaacagtaagctgctgaatcaatacc

SEQ ID NO: 570 - DNA, 202 bp, Homo sapiens
taagttgttgtgtatttcatcagcattcattcctcactatattaaagtaactaacttccctggagctccatcttttctgcctacttagtagtttacttgtagtaataagacattgaaacagtaagctgctgaatcaataccaagtaagcaaaaatcagctgcaagaaatgcagatctattgctaacattaatttttaaggca

SEQ ID NO: 571 - DNA, 202 bp, Homo sapiens
ataacttattctactctggatcacaccaaatactgcctgttagccactttatacatgcaaattggtcttttattaatgaattgtttaaaccaaaaaaatggtctccctaataacctaaaaatgattagcctcactttgtaaaaatacagcttacctatttgatatacacctactggcatgaatgatcttaagagactaattt

SEQ ID NO: 572 - DNA, 202 bp, Homo sapiens
ttcaggatataataataattgacaattgtgggaattgacaatttggattctggaaactaaatgagtatttttttttcaagaattttctcaatctctatgcagtttataagtagaacatgttcctgcttctgtattttctatgctatcatttcaactcctttttcattccttatagaccattaagttccaccagatcttacca

SEQ ID NO: 573 - DNA, 202 bp, Homo sapiens
gacaattgtgggaattgacaatttggattctggaaactaaatgagtatttttttttcaagaattttctcaatctctatgcatttataagtagaacatgttcgctgcttctgtattttctatgctatcatttcaactcctttttcattccttatagaccattaagttccaccagatcttaccatttcaatctcttaacagcct

SEQ ID NO: 574 - DNA, 202 bp, Homo sapiens
attctggaaactaaatgagtatttttttttcaagaattttctcaatctctatgcatttataagtagaacatgttcctgcttctgtattttctatgctatcagtttcaactcctttttcattccttatagaccattaagttccaccagatcttaccatttcaatctcttaacagccttctccttcaccccatgcttccgctga

SEQ ID NO: 575 - DNA, 202 bp, Homo sapiens
aacagccttctccttcaccccatgcttccgctgagtgcaagtactctctatctcttgcttaaacaattataacagccttttaatttatccttctgcctgtagatctggctgtattccaagccatcatgctcacttctactgaagagatttttctcaaaagcaaagtagagcctacaaggaaaagtccaaattcctttgccca

SEQ ID NO: 576 - DNA, 202 bp, Homo sapiens
ctttctcctcttctgtgctgtcatactgagctgctgatcatttctttaatgcatcttgatgtttcatgcttttctgcctttacccctgctgttccttctgctctggaattccctccccccaccttgtctatctgcccaacacctgctcagttttcatgatccaactcaagtgctatgtcctcccaggaaaccttttctgacc

SEQ ID NO: 577 - DNA, 204 bp, Homo sapiens
ataaacttctgtttggttgatttttatacatcgccatattcatctttgtagctccggaaacttagcagtgcttggaaaagtgaaagtcattgaatgaatataaaaaagcaagggcaggaggaaacaggtctccaaagccaacagttttatttcatttgtggcaaagtgccattcgatttaaattcaaagccattttgataggca

SEQ ID NO: 578 - DNA, 202 bp, Homo sapiens
gttcctccagaagctcactggatggcaagagttcaagtacaagtaagatagagggaggtgaaaacacaattaggggaatgggaagctgaggtagggaaggcgaaggcagcctgtgaaggatgcattcttaagccagttatcattgtggctcactgagtcagttcagatcatgcacctcagaattacccctcctagaggcaga

SEQ ID NO: 579 - DNA, 202 bp, Homo sapiens
agatagagggaggtgaaaacacaattaggggaatgggaagctgaggtagggaaggcaaggcagcctgtgaaggatgcattcttaagccagttatcattgtcggctcactgagtcagttcagatcatgcacctcagaattacccctcctagaggcagatggggtattcatacgctaactcccattggtcattgattgagggtt

SEQ ID NO: 580 - DNA, 202 bp, Homo sapiens
ggctgagggggctgtggtagaccttgacatggcagctacatctggtctttgatgccattgagtggtcattgtaaatataacaggataaaatgtgatatttactcataatctctcagcttacatatacctcagactatttgctcagttcaatattaattagggaaaggagcagaacattttgttcctcattacattaagacaa

SEQ ID NO: 581 - DNA, 203 bp, Homo sapiens
agctacatctggtctttgatgccattgagtggtcattgtaaatataacaggataaaatgtgatatttctcataatctctcagcttacatatacctcagacgactttgctcagttcaatattaattagggaaaggagcagaacattttgttcctcattacattaagacaatttgtgaaatatgtccagggtgagaatatccact

SEQ ID NO: 582 - DNA, 203 bp, Homo sapiens
ttcaatattaattagggaaaggagcagaacattttgttcctcattacattaagacaatttgtgaaatatgtccagggtgagaatatccactaaaaaacacactgtcaaagtgattctcttgggttgctattgttcaatctgagtatgttgtaatcataattctaaagtcattatgctcaaaccctttttgcctttgccaaatt

SEQ ID NO: 583 - DNA, 202 bp, Homo sapiens
gctcaaaccctttttgcctttgccaaattatctcacctgaattaacaggtaaccaagatgaaaataatttacaatttacaattggtcccattaaggacctagtagagagacgcacatgactgacagctcaaatataactatgccaggctgctaggctttgtgtggcccaacaagctgtgaaaggcacagaaatatgcaaatc

SEQ ID NO: 584 - DNA, 202 bp, Homo sapiens
aatttacaattggtcccattaaggacctatagagagacgcacatgactgacagctcaaatataactatgccaggctgctaggctttgtgtggcccaacaacgctgtgaaaggcacagaaatatgcaaatcagtagatgacgaatccctaggtattaattagaacttacaaatatattattctcctggaagattttgtcttgt

SEQ ID NO: 585 - DNA, 202 bp, Homo sapiens
attgcttacttattggagataaatataaatctggggaaatcactcataaagaagtaactccaacagttgtatcaataactgaagtttcatttgtcaactgagttatttttttttctgtgatacaatcagatcttttctgaatcctgcacttaaaacattttaaaaacttgagggcactttgaattaaaccaccaattagttt

SEQ ID NO: 586 - DNA, 202 bp, Homo sapiens
taactccaacagttgtatcaataactgaagtttcatttgtcaactggttatttttttttctgtgatacaatcagatcttttctgaatcctgcacttaaaactattttaaaaacttgagggcactttgaattaaaccaccaattagttttacttttttaataaacatttctgatctcaagcagcttacaatcaaggaaaggac

SEQ ID NO: 587 - DNA, 202 bp, Homo sapiens
taagaaattaagacaattacaaataaatgtgtcaggggaaaggaagctcaagaccactatgagatagataacaaatagtgttaaaagttgcatagacaaaagtatggaatgggatttcagagaagagaaagagctgagatctggaaatgtcagggagggctttatggagtgaccacaggtcctgtgtgcctgggacagttct

SEQ ID NO: 588 - DNA, 202 bp, Homo sapiens
aaaacctatctgtttgtatgacagattattgggtctcttcattcacaaagcgaaatggaaagaaacaataggattgggactgctatggaaaatggtagtgagccccgagggtaataaagggcatgagtagaagtcaccagggtggatatgaacgtcatgtgcaggagacacaaagagcctgtacttgctgtattagaattcc

SEQ ID NO: 589 - DNA, 202 bp, Homo sapiens
gctgggtatggaggagcaacattgtggggggggtattcctgaagatccagctcagacagaaacaagatcttattttcctattttcaagtcactagaaatgctacttgcccattcatgttattttctattagctttccattcaaagtcctacacatatacgataactggtgtcctggattggaagagaaaagaattttccagg

SEQ ID NO: 590 - DNA, 202 bp, Homo sapiens
gctcagacagaaacaagatcttattttcctattttcaagtcactagaaatgcacttgcccattcatgttattttctattagctttccattcaaagtcctactacatatacgataactggtgtcctggattggaagagaaaagaattttccaggtatctgtcatctccacagaaatgctttgtctttgcagtgcttaggttgc

SEQ ID NO: 591 - DNA, 202 bp, Homo sapiens
ctcagacagaaacaagatcttattttcctattttcaagtcactagaaatgcacttgcccattcatgttattttctattagctttccattcaaagtcctacagcatatacgataactggtgtcctggattggaagagaaaagaattttccaggtatctgtcatctccacagaaatgctttgtctttgcagtgcttaggttgcc

SEQ ID NO: 592 - DNA, 202 bp, Homo sapiens
ttaggttgccactcctgtcctctttgagagtgaactctgaagaagctgtgttcgggaaggggaacatcacacaccggggcctgtcgtggggtgggggcagcgggggagggatagcattaggagatatacctaatgtaaatgatgagttaatgggtgcagcacaccaacatggcacatgtagacatatgtgacaaacctgcac

SEQ ID NO: 593 - DNA, 202 bp, Homo sapiens
cctgtcgtggggtgggggcagcggggagggatagcattaggagatatacctaatgtaaatgatgagttaatgggtgcagcacaccaacatggcacatgtagtacatatgtgacaaacctgcacgttgtgcacatgtaccctagaacttaaagtataataaaaaaaaaaaaaaaaaagaagaagttgtgttctgggttggaag

SEQ ID NO: 594 - DNA, 202 bp, Homo sapiens
gcccaaagggccatatagtgaggagttgatcaaatttattgttcaggtggaaacttgagttcttctaagtcaactcgttcctacaagtgtcagtcatgttctgttcaattttttaaagtctctcttttgaccctagcttccctcattatttgaatttttttaaacagccaaattacatataccccaaagttattccccactc

SEQ ID NO: 595 - DNA, 202 bp, Homo sapiens
aagttattccccactctgactcagcttcctttccttccactcccacatcaaccccactagccttgctccacttcattcttcttctatatctgcaatctagacaacctcaccaaggagctgcaaacttttcatctatttgttccacactcctcaccatcatttaagactgcagatgagcttcttcttctgagaactcctctct

SEQ ID NO: 596 - DNA, 202 bp, Homo sapiens
atttgttccacactcctcaccatcatttaagactgcagatgagcttcttcttctgagaactcctctcttatgaattctaagacaaatcaccattgtctgcgtttttatttttcggaacactttttctgtgacatcttccatgcacttccttggcaattttttcctcttcaaatggttaagccttagctcagaaatctacaac

SEQ ID NO: 597 - DNA, 202 bp, Homo sapiens
cagaattttctaggtacttccagaaagaatcatccctgagtctcttgtcaggcttttgctcatactcccccatgttaagggctccactagtttttgttttatgtttttgtttttttcacctgggctccaaaccctacagatactgttggttctttccctctgcaaaactctcctacccatccacaaggcacaacctgcacat

SEQ ID NO: 598 - DNA, 202 bp, Homo sapiens
ttcacctgggctccaaaccctacagatactgttggttctttccctctgcaaaactctcctacccatccacaaggcacaacctgcacatcccatctccattagtagctttcactttggcttatttttcatatggacattagacccccgttaccacttctgtgcatagttacaaaagctaggaactggttctactacctacagt

SEQ ID NO: 599 - DNA, 202 bp, Homo sapiens
ttctttccctctgcaaaactctcctacccatccacaaggcacaacctgcacatcccatctccattatagctttcactttggcttatttttcatatggacacttagacccccgttaccacttctgtgcatagttacaaaagctaggaactggttctactacctacagtctctcactgtactttctacagtgcgctattactct

SEQ ID NO: 600 - DNA, 202 bp, Homo sapiens
tctccattatagctttcactttggcttatttttcatatggacattagacccccgttaccacttctgtgcatagttacaaaagctaggaactggttctactagcctacagtctctcactgtactttctacagtgcgctattactctctaccaaatgtgagatctgtttttgtaacttgcatctccaacatgccattcctttag

SEQ ID NO: 601 - DNA, 202 bp, Homo sapiens
ttctgtcaatggcttgccttcagctgtggctttctctgggctctagcaactatcccttctgcgttcctggtttgcctacccaatgtagggccacaatacactcatgatcccttaccactttctctcaattctgcccacagttttgtacaatgttcctttgttcaactcttttcaactgcccctttagaatatgccatcattt

SEQ ID NO: 602 - DNA, 202 bp, Homo sapiens
ttgtactcctcaaaacaacatcctacatttttttcacatgatgtcttggctttgagttgttaccactgtatagaattgagagaccataagcatagtggggacaaaaagccccaaactcccaaaacatgagtctagtgtaagctatgccatttgctagccatgtggctttggaaaaatgacttaacctttccaagcacagttt

SEQ ID NO: 603 - DNA, 202 bp, Homo sapiens
caacatcctacatttttttcacatgatgtcttggctttgagttgttaccactgtatagaattgagagaccataagcatagtggggaaaaaagccccaaacctcccaaaacatgagtctagtgtaagctatgccatttgctagccatgtggctttggaaaaatgacttaacctttccaagcacagtttcttcacctgtaaaat

SEQ ID NO: 604 - DNA, 202 bp, Homo sapiens
ataaattaccccactccactcatccttcctattatgtctaatcaaatgcaaaataggtaaacatttgaatgactaaatgctgctcttttgctgtggctccctggggttcccatgaaagaatttcatggaggtaaagtaatcagaaccggtattgggtgttgcactagtaacaaagtgttactcatgctaaaaagccaacagg

SEQ ID NO: 605 - DNA, 202 bp, Homo sapiens
gactaaatgctgctcttttgctgtggctcccggggttcccatgaaagaatttcatggaggtaaagtaatcagaaccggtattgggtgttgcactagtaacacaagtgttactcatgctaaaaagccaacaggcttctcaggggagtcttattttgaagactataatcattttccagaagcatgtgctcattcaacccatatt

SEQ ID NO: 606 - DNA, 202 bp, Homo sapiens
ataggagctcaaaccctactgtgaactgcatgtatgagggatctaggttgcatgctccttatgagaacctaactaatgcctgacgatctgacgtggaaccagtttcatcctgaaaccatcacccccaacctggtctgtagaaaaattttcctccacaaaactggtccctggtgccaaaatggttggggacctctgctttaga

SEQ ID NO: 607 - DNA, 202 bp, Homo sapiens
acagatgaaatgaccattttaaagtcagtcatccatgacagaaccaggaattagaacccaggtcacccaactacatcttagagtgttatcaacatagtttagcagacataacttaaggtgtacatcttaaactgtgtgacagtctaatcaatgcacaaaattcattctttttaatttttaagaagatgtagttggtacttgc

SEQ ID NO: 608 - DNA, 202 bp, Homo sapiens
tgaccattttaaagtcagtcatccatgacagaaccaggaattagaacccaggtcacccaactacatcttagagtgttatcaacatagtttacagacataaacttaaggtgtacatcttaaactgtgtgacagtctaatcaatgcacaaaattcattctttttaatttttaagaagatgtagttggtacttgctgaaagcagt

SEQ ID NO: 609 - DNA, 202 bp, Homo sapiens
tatcatgtgagctgaagtgctaaaattgctaaagtatgactgtaagcagttaatttcatcccgagtcttgtccacacacaatcaaatgcaaatttccactagtctcctgagaaactgccaagagatgactgaaatggaatagcaacaacctcctttatgtggaatcattatctggtcatctttgcttttttacataacttgt

SEQ ID NO: 610 - DNA, 202 bp, Homo sapiens
cacatttgctggcagatgcactaagtctgataaactcagcaagactgaatctcaccctctgaactgaaaaactttgaatggacctttgaaaacggtagaaattgacaatggttagctgcaagtgatattttcaaggcaaacagacactctcccaaagtattaaataacccagcattctaagttgcaggtggaaggtagccat

SEQ ID NO: 611 - DNA, 202 bp, Homo sapiens
cctacataaaagagattctgcaatgggcagcactaacatgaaacagtgttcagaagtacccattttccctcagattctaaactgacaaggtttccacttactcaggttatgaagttctaaagctgcaagacatccttgaggtcatcacaggatatttatttattttttcttcgggtgcatccaatagttatcaacttttcct

SEQ ID NO: 612 - DNA, 202 bp, Homo sapiens
cagattctaaactgacaaggtttccacttaccaggttatgaagttctaaagctgcaagacatccttgaggtcatcacaggatatttatttattttttcttctgggtgcatccaatagttatcaacttttcctcctctttaaaagctacttaaatctcattgaagttttgttttgttttgtttttgaaatctaagtaatgaga

SEQ ID NO: 613 - DNA, 202 bp, Homo sapiens
caggaaataaagtagcatgttcctgaataattcccccacccccttttattttcctggtagtcaggcttcctccaaaataccttatttgacctttatacctgttagaaacagcaagtgcctaattcgcctctgtgggttgctaatccgatttacgtgagcggaacctagtattattttagctcccctaccgaaaaaataatac

SEQ ID NO: 614 - DNA, 202 bp, Homo sapiens
agtcaggcttcctccaaaataccttatttgacctttatacctttagaaacagcaagtgcctaattcgcctctgtgggttgctaatccgatttacgtgagcaggaacctagtattattttagctcccctaccgaaaaaataatacacatggataatagttctattaccagctcctgcttctgacttttttctctctgtttcgc

SEQ ID NO: 615 - DNA, 202 bp, Homo sapiens
aatccgatttacgtgagcggaacctagtattattttagctcccctaccgaaaaaataatacacatggataatagttctattaccagctcctgcttctgaccttttttctctctgtttcgcaggcccgatagctctgggaaagcagaacttggccttttccaaaaattttctgcccttggttttggggatcatttgggcaagc

SEQ ID NO: 616 - DNA, 202 bp, Homo sapiens
agaacttggccttttccaaaaattttctgcccttggttttggggatcatttgggcaagcccgaggtgctgtgcatgggggctcctggaatcctgggaaggagcagaaagccttggccccagactcatcgtgcagcagctctgagcagtatttcggctgaggagtgacttcagtgaatattcagctgaggagtgacttggcca

SEQ ID NO: 617 - DNA, 202 bp, Homo sapiens
aaaattttctgcccttggttttggggatcatttgggcaagcccgaggtgctgtgcatgggggctcctggaatcctgggaagggcagaaagccttggccccacgactcatcgtgcagcagctctgagcagtatttcggctgaggagtgacttcagtgaatattcagctgaggagtgacttggccacgtgtcacagccctactt

SEQ ID NO: 618 - DNA, 202 bp, Homo sapiens
gctcctggaatcctgggaagggcagaaagccttggccccagactcatcgtgcagcagctctgagcagtatttcggctgaggagtgacttcagtgaatattctagctgaggagtgacttggccacgtgtcacagccctacttcttgggggcctggtggaagagggtggcgtagaaggttccaaggtcccaaactggaattgtc

SEQ ID NO: 619 - DNA, 202 bp, Homo sapiens
gaaggttccaaggtcccaaactggaattgtcctgtatgcttggttcacacagtgcgttattttaccttcctctgagctgctaatcgcctgcctctgagctagggtgagataaatatcacaaggcacaaagtgattgtacaataaaaaaatcaaatccctcccatccatccttcagtctgccacacacgcagtctacgttaca

SEQ ID NO: 620 - DNA, 202 bp, Homo sapiens
gttcacacagtgcgttattttaccttcctctgagctgctaatcgcctgcctctgagctgggtgagataaatatcacaaggcacaaagtgattgtacaataaatcaaatccctcccatccatccttcagtctgccacacacgcagtctacgttacacacatgtcacgtaaagcaggatgacatccatgtcacatacatagaca

SEQ ID NO: 621 - DNA, 202 bp, Homo sapiens
gggtgagataaatatcacaaggcacaaagtgattgtacaataaaaaaatcaaatccctcccatccatccttcagtctgccacacacgcagtctacgttacagcacatgtcacgtaaagcaggatgacatccatgtcacatacatagacatattaaccgaaatgtggcccttcggttgcatatattctcatacatgaatatat

SEQ ID NO: 622 - DNA, 202 bp, Homo sapiens
atccctcccatccatccttcagtctgccacacacgcagtctacgttacacacatgtcacgtaaagcaggatgacatccatgtcacatacatagacatattagaccgaaatgtggcccttcggttgcatatattctcatacatgaatatatttatagaaatatatgcacatatttttgtatattggatatatttatgtaacta

SEQ ID NO: 623 - DNA, 202 bp, Homo sapiens
atttttgtatattggatatatttatgtaactataaatttacatgcgtatggatatgaaaataaatgcatacacatttatgtaaaaaaatttgtacacatgctatttacatatgtaaatacatacatctctatgtattaatgtttaaaaacactcaatttccagcctgctgttttcttttaattttcctcctattccggggaa

SEQ ID NO: 624 - DNA, 202 bp, Homo sapiens
cgcgcccccgccctccgccccaccccgccgcggctgcccgggcgcgccgtccacacccctgcgcgcagctcccgcccgctcggggatccccggcgagccgacgccgcgaagggggaggtgttcggccgcggccgggagggagccggcaggcggcgtcccctttaaaagccgcgagcgccgcgccacggcgccgccgccgccg

SEQ ID NO: 625 - DNA, 202 bp, Homo sapiens
gccgagtggatcaccggggaccgcgaggcacccgcgcgccgcagaccccgcgcgggctggagcacccggcagagcgcgccacagcgccgtggcctctgctcgcccgggctgcgccagagccgcggacgggcgcgcagagcgccggggactccggagccgatccctagcgccgcgatgcggagcacctactgcaggagatcgg

SEQ ID NO: 626 - DNA, 202 bp, Homo sapiens
cttggccccagggctaggagagcgaggggagagcacagccacccgcctcggcggcccgggactcggctcgactcgccggagaatgcgcccgaggacgacgagggcgccagagccgcggtgctttcaactggcgagcgcgaatgggggtgcactggagtaaggcagagtgatgcgggggggcaactcgcctggcaccgagatc

SEQ ID NO: 627 - DNA, 202 bp, Homo sapiens
gcagagtgatgcgggggggcaactcgcctggcaccgagatcgccgccgtgcccttccctggacccggcgtcgcccaggatggctgccccgagccatgggcctgcggcggagctagcgcggagcgcccgaccctcgacccccgagtcccggagccggccccgcgcggggccacgcgtccctcgggcgctggttcctaaggagg

SEQ ID NO: 628 - DNA, 202 bp, Homo sapiens
accgagatcgccgccgtgcccttccctggacccggcgtcgcccaggatggctgccccgagccatgggccgcggcggagctagcgcggagcgcccgaccctcggacccccgagtcccggagccggccccgcgcggggccacgcgtccctcgggcgctggttcctaaggaggacgacagcaccagcttctcctttctcccttcc

SEQ ID NO: 629 - DNA, 203 bp, Homo sapiens
acgcgtccctcgggcgctggttcctaaggaggacgacagcaccagcttctcctttctcccttcccttccctgccccgcactcctccccctgctcgctgtttgttgtgtgtcagcacttggctggggacttcttgaacttgcagggagaataacttgcgcaccccactttgcgccggtgcctttgccccagcggagcctgcttc

SEQ ID NO: 630 - DNA, 202 bp, Homo sapiens
gccttgcccgacactgagacgctgttcccagcgtgaaaagagagactgcgcggccggcacccgggagaaggaggaggcaaagaaaaggaacggacattcggttccttgcgccaggtcctttgaccagagtttttccatgtggacgctctttcaatggacgtgtccccgcgtgcttcttagacggactgcggtctcctaaagg

SEQ ID NO: 631 - DNA, 202 bp, Homo sapiens
aacggacattcggtccttgcgccaggtcctttgaccagagtttttccatgtggacgctctttcaatggacgtgtccccgcgtgcttcttagacggactgccggtctcctaaaggtagaggacgcgggccagggcccggggtgggtggtgggtgggagggggatttgggcagccactgcggtagagcccttccttacgtccag

SEQ ID NO: 632 - DNA, 202 bp, Homo sapiens
atttgggcagccactgcggtagagcccttccttacgtccaggccagaagtaaacagacccctctccagtccacgtgcaacggagccctgcaggggctcccaccttccagctgccccgggcgaccgtaagcctcaccctcccggcccgcactcttccacccctctttcttcccctctccctggaatacttttggagctgttaa

SEQ ID NO: 633 - DNA, 202 bp, Homo sapiens
ttacgtccaggccagaagtaaacagacccctctccagtccacgtgcaacggagccctgcaggggctcccacttccagctgccccgggcgaccgtaagcctctaccctcccggcccgcactcttccacccctctttcttcccctctccctggaatacttttggagctgttaacacttagatgaggtgttttatttatttattt

SEQ ID NO: 634 - DNA, 202 bp, Homo sapiens
cttccacccctctttcttcccctctccctggaatacttttggagctgttaacacttagatgaggtgttttatttatttatttatttatttttaattttttataaaaacttttttgggtcaaagaaatccctttgagagggtagcccctgggtttcacccgttagctgagaacctgtccgctctgccatggtgatctccattc

SEQ ID NO: 635 - DNA, 202 bp, Homo sapiens
ccgctctgccatggtgatctccattcttcaagtgtttccgggagacttggtttctttgctcagagccgtgtcccatttaggaaagtactaggagtttgggagttctccctacttgtttccagaaatgcgaggggtcagtactgaaggatcacttggtactgtgtttttaacagctgacacgtgcattaatagatattcacca

SEQ ID NO: 636 - DNA, 202 bp, Homo sapiens
gctctgccatggtgatctccattcttcaagtgtttccgggagacttggtttctttgctcagagccgtgtcccatttaggaaagtactaggagtttggggtgtctccctacttgtttccagaaatgcgaggggtcagtactgaaggatcacttggtactgtgtttttaacagctgacacgtgcattaatagatattcaccatt

SEQ ID NO: 637 - DNA, 202 bp, Homo sapiens
ctgccatggtgatctccattcttcaagtgtttccgggagacttggtttctttgctcagagccgtgtcccatttaggaaagtactaggagtttggggttctctcctacttgtttccagaaatgcgaggggtcagtactgaaggatcacttggtactgtgtttttaacagctgacacgtgcattaatagatattcaccatttac

SEQ ID NO: 638 - DNA, 202 bp, Homo sapiens
cgggagacttggtttctttgctcagagccgtgtcccatttaggaaagtactaggagtttggggttctccctacttgtttccagaaatgcgaggggtcagtacctgaaggatcacttggtactgtgtttttaacagctgacacgtgcattaatagatattcaccatttacgtaatcccgggaagatacatgtgtatcttgact

SEQ ID NO: 639 - DNA, 202 bp, Homo sapiens
tgacacgtgcattaatagatattcaccatttacgtaatcccgggaagatacatgtgtatcttgactgcactgtggggatgcgggatggagctgcctttcgaggacacccctgagggtaggggcctgggacacaagtcataagtggcttcagaagttgtggccttgagcttacagggtctggaagctataagggtgtgtgtgt

SEQ ID NO: 640 - DNA, 202 bp, Homo sapiens
agggtaggggcctgggacacaagtcataagtggcttcagaagttgtggccttgagcttacagggtctggaagctataagggtgtgtgtgtgtgtgtgtgtgttgtgtgtgtgtgtgtcaggaagttctatacagtgcctctaaggaagtcacatgcaccatttatgtgtgtttatatgccagacagcgctcagcactccgca

SEQ ID NO: 641 - DNA, 202 bp, Homo sapiens
gctgtgtcacacacaaaaaagtgacagtcattggcgctggtttggttgggggggagggcaaatcccaaatctgatgtcagacgagctaagcgttggatggagagcgataaatcatctggttcaggaacttgggacccttcattatcccaaacgtttgagcttcggtcggtcttacctagactcgtgagtgtgccaagccagg

SEQ ID NO: 642 - DNA, 202 bp, Homo sapiens
cgggacccgctgtcttctagcgttgctgcttccccaggtcctcctgggcggcgcggctggcctcgttccggagctgggccgcaggaagttcgcggcggcggtcgtcgggccgcccctcatcccagccctctgacgaggtcctgagcgagttcgagttgcggctgctcagcatgttcggcctgaaacagagacccacccccag

SEQ ID NO: 643 - DNA, 202 bp, Homo sapiens
agttgcggctgctcagcatgttcggcctgaaacagagacccacccccagcagggacgccgtggtgcccccctacatgctagacctgtatcgcaggcactcagggtcagccgggctcacccgccccagaccaccggttggagagggcagccagccgagccaacactgtgcgcagcttccaccatgaaggtgaggcatggagca

SEQ ID NO: 644 - DNA, 202 bp, Homo sapiens
gttcggcctgaaacagagacccacccccagcagggacgccgtggtgcccccctacatgctagacctgtatcgcaggcactcaggtcagccgggctcacccgtccccagaccaccggttggagagggcagccagccgagccaacactgtgcgcagcttccaccatgaaggtgaggcatggagcagggcgtgggggcggggagt

SEQ ID NO: 645 - DNA, 202 bp, Homo sapiens
gggcaccagcggacgtttccactcttgcttctgtactatcgtttctgaatctgattttaactcactgcttgtgtggtgggggagccagggattcccctttaggtaactccgcaccctcttcctggcttgcagccagaagagctactcctcctggaagaattggagagaaatcaagtgatggggaagatgagggcaaaaggca

SEQ ID NO: 646 - DNA, 202 bp, Homo sapiens
actgcttgtgtggtgggggagccagggattcccctttagtaactccgcaccctcttcctggcttgcagccagaagagctactcctcctggaagaattggaagagaaatcaagtgatggggaagatgagggcaaaaggcatgcctctagtcagctaaacgtgcaagaattccacagagggaaaaggagaaaaagggaggcaga

SEQ ID NO: 647 - DNA, 202 bp, Homo sapiens
aatcaagtgatggggaagatgagggcaaaaggcatgcctctagtcagctaaacgtgcaagaattccacagagggaaaaggagaaaaagggaggcagattgaggatttctttaagtctgtttggaagcttttgctctataaatctgccgcttaagccagggttttagggtagacagagccaagggcagagttttcagagatag

SEQ ID NO: 648 - DNA, 202 bp, Homo sapiens
ggggaagatgagggcaaaaggcatgcctctagtcagctaaacgtgcaagaattccacagagggaaaaggagaaaaagggaggcagattgagatttctttaatgtctgtttggaagcttttgctctataaatctgccgcttaagccagggttttagggtagacagagccaagggcagagttttcagagatagtattgaaaaat

SEQ ID NO: 649 - DNA, 202 bp, Homo sapiens
gggaggcagattgagatttctttaagtctgtttggaagcttttgctctataaatctgccgcttaagccagggttttagggtagacagagccaagggcagaagttttcagagatagtattgaaaaatcaaagcccagggccccaaagtctttctaatttatagttgatctgggcctggtttggaagattttgaatcccaatct

SEQ ID NO: 650 - DNA, 202 bp, Homo sapiens
ttctaatttatagttgatctgggcctggtttggaagattttgaatcccaatctaatccccgtgggagatcaatactacaatcaatcttattgtttccacaagtgactttcttgtcctgtgcttaaatctgagataggctctgagtagagacaaggcaagccttcagataaaagcgtttgtagcagctgcctgtttttttttc

SEQ ID NO: 651 - DNA, 202 bp, Homo sapiens
ttaaatctgagataggctctgagtagagacaaggcaagccttcagataaaagcgtttgtagcagctgcctgtttttttttcatgtgcaccgaaatgtggattcttttatgatactacatgtggtttttctaaggtgggatatttctgcttgtttcatcagaagggcatttagtggactggaaatgtcttacagcagctattg

SEQ ID NO: 652 - DNA, 202 bp, Homo sapiens
attctttttctacacttttaaatgcttctttggcttaatacatttaaaatagagcatgggtttcttcaattcctagaaaagagtacaaaagtgtatatcacgagagcaaccacttggcagatatttggggagttgggagtgaagttctctttcttgcctttccctgcttaggtggtaaatttcaagtgggaaatttacactg

SEQ ID NO: 653 - DNA, 202 bp, Homo sapiens
agaaaagagtacaaaagtgtatatcacagagcaaccacttggcagatatttggggagttgggagtgaagttctctttcttgcctttccctgcttaggtggataaatttcaagtgggaaatttacactgataatagactaatgggaaatggcacttccagatgttttctcccagtgtgaagggtgacttatacttgtgagagt

SEQ ID NO: 654 - DNA, 202 bp, Homo sapiens
agtacaaaagtgtatatcacagagcaaccacttggcagatatttggggagttgggagtgaagttctctttcttgcctttccctgcttaggtggtaaatttctaagtgggaaatttacactgataatagactaatgggaaatggcacttccagatgttttctcccagtgtgaagggtgacttatacttgtgagagtatttgtt

SEQ ID NO: 655 - DNA, 202 bp, Homo sapiens
tctcattgaggcagctacacattacgacggggacactgaattgatcatcagttcatttacaagcacatttctaagtgaggtgctctctgctagcagaaatacagatttgaaaggcagtaagatctcactccactctttcagaattcatccaatgaaagcagaaatcacctgttgtcatatgtaaaatttgtgtgtatgtgta

SEQ ID NO: 656 - DNA, 202 bp, Homo sapiens
catttctaagtgaggtgctctctgctagcagaaatcagatttgaaaggcagtaagatctcactccactctttcagaattcatccaatgaaagcagaaatcatcctgttgtcatatgtaaaatttgtgtgtatgtgtacattctgccatcttaaccctgaaatgattatagatccagctaatcattcccaggtaatgctgatt

SEQ ID NO: 657 - DNA, 202 bp, Homo sapiens
aagtggtttgatcagaactaaacaggctacacgttattcaactgtgttattttaacttaaaaagcatgcttgagtttataaaatcagaatttatatctttagtgagtgtaaatgttacctgagaaacagtacagaagtgaccaacttgattaaaatcaacttgtaataacttcaggtcttaatgcagttagataatggagaa

SEQ ID NO: 658 - DNA, 202 bp, Homo sapiens
gtaataacttcaggtcttaatgcagttagataatggagaaaagctatgtaattttgccccaaatttcaactaatccatttcttgtctcattatgactaatactatcatccttaatctggatggatatagcacttttttcaagactaatcattgttgtatacacccaggatttgcttttgataaacatccttgtgccatgcat

SEQ ID NO: 659 - DNA, 202 bp, Homo sapiens
acagcattaatgagcatttatttgaaaaaagaccataaagacccaatcataagaattacctgttgggttttctttgtaggtgtgatcgaatggtttggtgagaattactcgacgagatatcatgatagcattctttcaaccaatatgagtataatgcgaccatatcataggggatctgagacagaattatcagttgtatttt

SEQ ID NO: 660 - DNA, 202 bp, Homo sapiens
ctaaatttctcttaatccctttgcagtgaactattttgcgttcttagacttgctctttgtgtattttcactgagacaataagagaatatttcatcattccagaaggtgttggtgttaagggtgggcagaggccaaatcagggttgttgatgacaaccatgctctctattcctttatttgccattcccttgttgtattttttt

SEQ ID NO: 661 - DNA, 202 bp, Homo sapiens
aggtgttggtgttaagggtgggcagaggccaaatcagggttgttgatgacaaccatgctctctattcctttatttgccattcccttgttgtattttttttataaatggaatgtttttaaccttttgtatttgatattttttttctccttgatcagttgtctgttattttattatctggaaaatcttatattatactcagcct

SEQ ID NO: 662 - DNA, 202 bp, Homo sapiens
caaccatgctctctattcctttatttgccattcccttgttgtattttttttaaaatggaatgtttttaaccttttgtatttgatattttttttctccttgattcagttgtctgttattttattatctggaaaatcttatattatactcagcctctttcattttgtgttagggcagtgacttccagccttactgattgccagc

SEQ ID NO: 663 - DNA, 202 bp, Homo sapiens
tttgacttttgcttaagtttttgagtcctcatgtatgttaggtagtgccatctagtagccagaaatttgggaactggctgggcatgatggctaatacctgctaatcccagcactttgggaggcttaggtgggtggatcacttgaggtcaggagttccagaccagcctggccaacatggtgaaaccacatctctactaaaata

SEQ ID NO: 664 - DNA, 202 bp, Homo sapiens
ccatctagtagccagaaatttgggaactggctgggcatgatggctaatacctgtaatcccagcactttgggaggcttaggtgggtggatcacttgaggtcagggagttccagaccagcctggccaacatggtgaaaccacatctctactaaaatataaaaaaatagccaggtatgatggcccatgcctgtaatccgagctaa

SEQ ID NO: 665 - DNA, 202 bp, Homo sapiens
atacctgtaatcccagcactttgggaggcttaggtgggtggatcacttgaggtcaggagttccagaccagcctggccaacatggtgaaaccacatctctacttaaaatataaaaaaatagccaggtatgatggcccatgcctgtaatccgagctaattgggaggctgagatgggaggactgcttgaacctgggaggtggagg

SEQ ID NO: 666 - DNA, 202 bp, Homo sapiens
cttaggtgggtggatcacttgaggtcaggagttccagaccagcctggccaacatggtgaaaccacatctctactaaaatataaaaaaatagccaggtatgagtggcccatgcctgtaatccgagctaattgggaggctgagatgggaggactgcttgaacctgggaggtggaggctgctgtgagccaagattgtgccactgc

SEQ ID NO: 667 - DNA, 202 bp, Homo sapiens
ggctgagatgggaggactgcttgaacctgggaggtggaggctgctgtgagccaagattgtgccactgcactccagcctgggcaacagagtgagaccctgtcgtcacaaaaacaagaaacaaaacaaaacaaaagacaagaaacctgagaagcgcagtagattcaattatatatatctacttttaatttgctagctctgtgac

SEQ ID NO: 668 - DNA, 202 bp, Homo sapiens
agccaagattgtgccactgcactccagcctgggcaacagagtgagaccctgtgtcacaaaaacaagaaacaaaacaaaacaaaagacaagaaacctgagaacgcgcagtagattcaattatatatatctacttttaatttgctagctctgtgaccttaggaaagttacataacctctctgaactgcaactgtttcatttaca

SEQ ID NO: 669 - DNA, 202 bp, Homo sapiens
gataatgatagtttttctctaattggtttgttgtgagataattcatataaagctgatggtgccagattacactcaaaaaaagcattcagctgtcattatcagttatgacttcttttgttaatgttatagcctttccttctctagggaaaaggaggccagagtggacctaggctgactgagagaattcagctcagtcttttga

SEQ ID NO: 670 - DNA, 202 bp, Homo sapiens
gactgagagaattcagctcagtcttttgaattattttgaggtagaggaatgattgatatagtatagattattaaattaggacttcacttttggagaaaaggttcagatatcattgttgtcttatttttcttcactttcccacatttttgcagccatagctccatccatttggttaagaacttagaagctcacaaactcgggt

SEQ ID NO: 671 - DNA, 202 bp, Homo sapiens
agggtttctgtgaacgtctaacttacctctgtgacctgtttctgcaaccaggggtgttgcaatggatgcttttgtcttgaggatgggacctttcaagaaactagattcactgaggtgcagtgggaaggtcagagaaagatcttcgtatcgcctattattatttgctcgtctattttttctcctttcttaaggccactaactg

SEQ ID NO: 672 - DNA, 202 bp, Homo sapiens
acctgtttctgcaaccaggggtgttgcaatggatgcttttgtcttgaggatgggacctttcaagaaacagattcactgaggtgcagtgggaaggtcagagagaagatcttcgtatcgcctattattatttgctcgtctattttttctcctttcttaaggccactaactgattctcctttgctaaggctgcctacttccactg

SEQ ID NO: 673 - DNA, 202 bp, Homo sapiens
caggggtgttgcaatggatgcttttgtcttgaggatgggacctttcaagaaacagattcactgaggtgcagtgggaaggtcagagaaagatcttcgtatcagcctattattatttgctcgtctattttttctcctttcttaaggccactaactgattctcctttgctaaggctgcctacttccactgagaccttgaaccaca

SEQ ID NO: 674 - DNA, 202 bp, Homo sapiens
agtgggaaggtcagagaaagatcttcgtatcgcctattattatttgctcgtctattttttctcctttcttaaggccactaactgattctcctttgctaagagctgcctacttccactgagaccttgaaccacatgaaattgttgttgtctgtgtttctggtcaaatagtggcaattttgtatgattcaatcttgtcatttaa

SEQ ID NO: 675 - DNA, 202 bp, Homo sapiens
gcactttaaaaatgcactcctggcaatctatactggaatatgaaaaacatgctgcaaaaccttgacactccaagtgtggtcttacagttcccagaatccccttccttgaggagctgctagaaatgctgaatctcaagcatctccccagacctactgaatcagagcctgcatctgaagctttacggtgtacaagctgttttat

SEQ ID NO: 676 - DNA, 202 bp, Homo sapiens
agctgttttatgtgaaggctgaagtttgaaaagcactgcattaaagcgttagtttggtataaactgccctgactgaacttggtgtgtccacttagcttgcagtgatgactgttgctttgatgatgaaggcttacacgggtagatcctttgagtgagtgatctgacatgattctcctttgctaaggcatctagattcagtgca

SEQ ID NO: 677 - DNA, 201 bp, Homo sapiens
gttgctttgatgatgaaggcttacacgggtagatcctttgagtgagtgatctgacatgattctcctttgctaaggcatctagattcagtgcacaacttacaagctgtttgtctttaggggaaatacaactgtaaaattaataaaaacatagtctcttcttatgataacatggaacgatggcaaaatagattttgttagcac

SEQ ID NO: 678 - DNA, 203 bp, Homo sapiens
aattctgttggcagtgaaatgataggatgtggtaaagttagaataaaataaacttaaatgtctcaaactctcatggtatatactaccagtttaataataatgttgtacctttgatgatttgcagactacaagcattcaaggtgctgtgttatatattacttgcttggagaataatacttcttaaaaattgaaattcagaaatt

SEQ ID NO: 679 - DNA, 202 bp, Homo sapiens
gaaatgataggatgtggtaaagttagaataaaataaacttaaatgtctcaaactctcatggtatatactaccagtttaataataatgttgtacctttgatgtatttgcagactacaagcattcaaggtgctgtgttatatattacttgcttggagaataatacttcttaaaaattgaaattcagaaattttaaatcagacaa

SEQ ID NO: 680 - DNA, 202 bp, Homo sapiens
gcatggcccacttaaatggctattttgaaataatgatagtggatatagaaggattattctgtaataggatgagactgttccttttgtcatggagatcataagtcatatttttgtaaatttttattatttttttggttttgtgtccatcctgcacactattactgggtaggtacatggttttttaacatggtttatctttcaa

SEQ ID NO: 681 - DNA, 202 bp, Homo sapiens
ttggttttgtgtccatcctgcacactattactgggtaggtacatggttttttaacatggtttatctttcaaaactataaaggcattgcaaacagaagacaaggtcatttatttttcttccaaaagcatctaaaatgagattttgatatttgaggtcataaagaggtgagagaacagacaacagttgggaaagctatttctct

SEQ ID NO: 682 - DNA, 202 bp, Homo sapiens
gggtaggtacatggttttttaacatggtttatctttcaaaactataaaggcattgcaaacagaagacaggtcatttatttttcttccaaaagcatctaaaagtgagattttgatatttgaggtcataaagaggtgagagaacagacaacagttgggaaagctatttctcttgaaattgtttggccttaattactacagtgtc

SEQ ID NO: 683 - DNA, 201 bp, Homo sapiens
aaaagcatctaaaatgagattttgatatttgaggtcataaagaggtgagagaacagacaacagttgggaaagctatttctcttgaaattgtttggccttaaattactacagtgtcctagtaccacccatacgtttccaaagaagtagatccctgtaaatgcctttgtctctggacttttgagtaaaatagtagggtgtgct

SEQ ID NO: 684 - DNA, 202 bp, Homo sapiens
gaggtcataaagaggtgagagaacagacaacagttgggaaagctatttctcttgaaattgtttggccttaattactacagtgtcctagtaccacccatacagtttccaaagaagtagatccctgtaaatgcctttgtctctggacttttgagtaaaatagtagggtgtgctttgcaaaatgtcatcgttgatgttgagtttc

SEQ ID NO: 685 - DNA, 202 bp, Homo sapiens
agtaccacccatacgtttccaaagaagtagatccctgtaaatgcctttgtctctggacttttgagtaaaatagtagggtgtgctttgcaaaatgtcatcgcttgatgttgagtttcagagtctttaattaggaagctgaaatctgtatatcgagatttgtaaatcatctaaattgcagagtaatgttttagaatactgctta

SEQ ID NO: 686 - DNA, 202 bp, Homo sapiens
tgtctctggacttttgagtaaaatagtagggtgtgctttgcaaaatgtcatcgttgatgttgagtttcagagtctttaattaggaagctgaaatctgtatagtcgagatttgtaaatcatctaaattgcagagtaatgttttagaatactgcttaagggattggcattaaagccttttttaaaaaagaaatgcaataatttc

SEQ ID NO: 687 - DNA, 202 bp, Homo sapiens
gtttatttaaagaggaaatttttattttctggttacctacttttacatgggttacatcaaatcccacgatgaggtttaaaaattctcatagataatcaaactgtcattacttggcttactgaaattcagacttttcttttttcttccctgtttttctctatcaaattagaatctttggaagaactaccagaaacgagtggga

SEQ ID NO: 688 - DNA, 202 bp, Homo sapiens
tgcaagatgctttaggaaacaatagcagtttccatcaccgaattaatatttatgaaatcataaaacctgcaacagccaactcgaaattccccgtgaccagatcttttggacaccaggttggtgaatcagaatgcaagcaggtgggaaagttttgatgtcacccccgctgtgatgcggtggactgcacagggacacgccaacc

SEQ ID NO: 689 - DNA, 202 bp, Homo sapiens
tgaactccactaatcatgccattgttcagacgttggtcaactctgttaactctaagattcctaaggcatgctgtgtcccgacagaactcagtgctatctcagatgctgtaccttgacgagaatgaaaaggttgtattaaagaactatcaggacatggttgtggagggttgtgggtgtcgctagtacagcaaaattaaataca

SEQ ID NO: 690 - DNA, 202 bp, Homo sapiens
ctaagattcctaaggcatgctgtgtcccgacagaactcagtgctatctcgatgctgtaccttgacgagaatgaaaaggttgtattaaagaactatcaggactatggttgtggagggttgtgggtgtcgctagtacagcaaaattaaatacataaatatatatatatatatatattttagaaaaaagaaaaaaacaaacaaac

SEQ ID NO: 691 - DNA, 202 bp, Homo sapiens
tgacgagaatgaaaaggttgtattaaagaactatcaggacatggttgtggagggttgtgggtgtcgctagtacagcaaaattaaatacataaatatatatagtatatatatattttagaaaaaagaaaaaaacaaacaaacaaaaaaaccccaccccagttgacactttaatatttcccaatgaagactttatttatggaat

SEQ ID NO: 692 - DNA, 202 bp, Homo sapiens
aaaacagctattttgaaaatatatttatatctacgaaaagaagttgggaaaacaaatattttaatcagagaattattccttaaagatttaaaatgtatttacgttgtacattttatatgggttcaaccccagcacatgaagtataatggtcagatttattttgtatttatttactattataaccactttttaggaaaaaaat

SEQ ID NO: 693 - DNA, 202 bp, Homo sapiens
catgaagtataatggtcagatttattttgtatttatttactattataaccactttttaggaaaaaaatagctaatttgtatttatatgtaatcaaaagaaagtatcgggtttgtacataattttccaaaaattgtagttgttttcagttgtgtgtatttaagatgaaaagtctacatggaaggttactctggcaaagtgctt

SEQ ID NO: 694 - DNA, 202 bp, Homo sapiens
taatcaaaagaagtatcgggtttgtacataattttccaaaaattgtagttgttttcagttgtgtgtatttaagatgaaaagtctacatggaaggttactcctggcaaagtgcttagcacgtttgcttttttgcagtgctactgttgagttcacaagttcaagtccagaaaaaaaaagtggataatccactctgctgactttc

SEQ ID NO: 695 - DNA, 202 bp, Homo sapiens
tgagttcacaagttcaagtccagaaaaaaaaagtggataatccactctgctgactttcaagattattatattattcaattctcaggaatgttgcagagtgagttgtccaatccatgagaatttacatccttattaggtggaatatttggataagaaccagacattgctgatctattatagaaactctcctcctgccccttaa

SEQ ID NO: 696 - DNA, 202 bp, Homo sapiens
attattcaattctcaggaatgttgcagagtgattgtccaatccatgagaatttacatccttattaggtggaatatttggataagaaccagacattgctgaatctattatagaaactctcctcctgccccttaatttacagaaagaataaagcaggatccatagaaataattaggaaaacgatgaacctgcaggaaagtgaat

SEQ ID NO: 697 - DNA, 202 bp, Homo sapiens
atgttgcagagtgattgtccaatccatgagaatttacatccttattaggtggaatatttggataagaaccagacattgctgatctattatagaaactctcactcctgccccttaatttacagaaagaataaagcaggatccatagaaataattaggaaaacgatgaacctgcaggaaagtgaatgatggtttgttgttcttc

SEQ ID NO: 698 - DNA, 202 bp, Homo sapiens
ttagtgatcccttcaaaggggctgatctggccaaagtattcaataaaacgtaagatttcttcattattgatattgtggtcatatatatttaaaattgatactctcgtggccctcatcaagggttggaaatttatttgtgttttacctttacctcatctgagagctctttattctccaaagaacccagttttctaactttttg

SEQ ID NO: 699 - DNA, 204 bp, Homo sapiens
tggccaaagtattcaataaaacgtaagatttcttcattattgatattgtggtcatatatatttaaaattgatatctcgtggccctcatcaagggttggaaatttatttgtgttttacctttacctcatctgagagctctttattctccaaagaacccagttttctaactttttgcccaacacgcagcaaaattatgcacatcgt

SEQ ID NO: 700 - DNA, 202 bp, Homo sapiens
ggccctcatcaagggttggaaatttatttgtgttttacctttacctcatctgagagctctttattctccaaagaacccagttttctaactttttgcccaactacgcagcaaaattatgcacatcgtgttttctgcccaccctctgttctctgacctatcagcttgcttttctttccaaggttgtgtgtttgaacacatttct

SEQ ID NO: 701 - DNA, 202 bp, Homo sapiens
tggcatttcattctataaagtccaacctgtaagagaaaatggtgcatttgtatagcgcttacaatgatgaccttgtgtttgcatttttgtttctgaagttattatattttagagggggtgggggaaaggtaatgaatggctggaaaattgcaggcaagttatttgataagtcatatttgcactaaaggtgttaccagtgatt

SEQ ID NO: 702 - DNA, 202 bp, Homo sapiens
gcacccacaactacccactattttactggcagtattttaggtcagtttccaggactttgcatcccctctgatcctgccatgcatgattggtgaaacctaccttctaatctccttggaattggctaaaaaacagtgtgtttataatggaacagactgttataatcaaattcttcctaggaattaacttttgatgactatgagc

SEQ ID NO: 703 - DNA, 202 bp, Homo sapiens
tattttaggtcagtttccaggactttgcatcccctctgatcctgccatgcatgattggtgaaacctacctctaatctccttggaattggctaaaaaacagatgtgtttataatggaacagactgttataatcaaattcttcctaggaattaacttttgatgactatgagcttagttacagttcggaggttatgaggttatgt

SEQ ID NO: 704 - DNA, 202 bp, Homo sapiens
catgacagttatcttttactaatgctggttaacttttaaaatcttgcagctcctttttatctctagttctattgttcttgattaggtgagaaccattagactcatacccaactgaggggattggggtcttgtttgttctccagctgttcttcaccctctattgccatggacatgaaggacagactgcacggtcttaacatgt

SEQ ID NO: 705 - DNA, 202 bp, Homo sapiens
tgttctccagctgttcttcaccctctattgccatggacatgaaggacagactgcacggtcttaacatgttaaaacgaatgacccatgttttctcatattcaccattctctagatgagagcatgagagtatagcacaagaatttcatcctttggcagttccatccacccttttgatttctctgcttcattggttctaatattc

SEQ ID NO: 706 - DNA, 202 bp, Homo sapiens
aatgacccatgttttctcatattcacattctctagatgagagcatgagagtatagcacaagaatttcatcctttggcagttccatccacccttttgatttcttctgcttcattggttctaatattcttggttaaaactgttttcaatggtgtcgactaaaatgtgttatcttgcatatgcgtatttaatatataaaacataa

SEQ ID NO: 707 - DNA, 202 bp, Homo sapiens
tagcacaagaatttcatcctttggcagttccatccacccttttgatttctctgcttcattggttctaatattcttggttaaaactgttttcaatggtgtcagactaaaatgtgttatcttgcatatgcgtatttaatatataaaacataaaatacataaatatacatctaaagatgtatgtaatatgtagtacatatgtata

SEQ ID NO: 708 - DNA, 202 bp, Homo sapiens
gtatagatgctggcattttcagtttagtttcagagattttataatcagacactctgaccctcttggaagctcacatgagaaacaatatttttacaaacatctctacattgttacagacattttagctcacagtcttcattccgaggaataaagagtcaaaatgaacataacactgaatgtctttaattgcactcactattct

SEQ ID NO: 709 - DNA, 202 bp, Homo sapiens
cctctggcatggttttaacctgtatggcacgtccgggttaccatgttcattatttgccttcaaaaattaggaaaagagagtgtgggcagcttacagatctagtctgtgcttccatcttttcataatttctacttaaatcgcagctttaaattgtaccttaagaggtcaagaatggagagatgatctgtaaacctgatctata

SEQ ID NO: 710 - DNA, 202 bp, Homo sapiens
ggtcaagaatggagagatgatctgtaaacctgatctatataacaagctgtataatcactacattacctaagcaatccatattttttccctcaatttattacttggagagggaaaatcatgtgcacaattcaaagggctttcaaaaatagcatatttctatgctactggcatattaatgttaagattcaccgaatgtttacgt

SEQ ID NO: 711 - DNA, 202 bp, Homo sapiens
tctaaggcaaaaggaacatagccttgcagtcaagagcaattaaagcctatagtaattgtggcatagattcataatccaagaaaagttaagaatgtcctggctgccatttctcttctgttttatatttatttatttatttatttatttatttttgtgcagggaacgggtagggtgaaatgggggataagatgcaggtctgctc

SEQ ID NO: 712 - DNA, 204 bp, Homo sapiens
ccttgcagtcaagagcaattaaagcctatagtaattgtggcatagattcataatccaagaaaagttaagaatgtcctggcgccatttctcttctgttttatatttatttatttatttatttatttatttatttttgtgcagggaacgggtagggtgaaatgggggataagatgcaggtctgctcagcttgggtaaattgctttt

SEQ ID NO: 713 - DNA, 203 bp, Homo sapiens
ctaaataaaactttaattttaatgtgcatccgaattaccaggaattctgtaaggagaataaaactttaacgtgtatacagacccccgggaatcatgttctattagagattctgtttcagtgagtctgggtggggcctgagatcctgcatttctaacaagctccgaggtgaagcccttacaggccaaaggaccacagattgagt

SEQ ID NO: 714 - DNA, 202 bp, Homo sapiens
tctgtgaatgtatatgtacatatgtaatgatttgttcctatctggagtccaaatgccaagcatagggcaccttcttctgttttactgagtttctcattttagcgtaggtctctgaatttcgcattgagtccctctgaggtgcttaagcagtactaagcaagacactgactttaacaccacttaaaagcttgcgtttgcccca

SEQ ID NO: 715 - DNA, 202 bp, Homo sapiens
atatgtaatgatttgttcctatctggagtccaaatgccaagcatagggcaccttcttctgttttactgagtttctcattttgcgtaggtctctgaatttcgtcattgagtccctctgaggtgcttaagcagtactaagcaagacactgactttaacaccacttaaaagcttgcgtttgccccatgtcctgggccccactcca

SEQ ID NO: 716 - DNA, 202 bp, Homo sapiens
ctgttttactgagtttctcattttgcgtaggtctctgaatttcgcattgagtccctctgaggtgcttaagcagtactaagcaagacactgactttaacacctacttaaaagcttgcgtttgccccatgtcctgggccccactccaaggcattgccagaagtacattattgttgcagtccattctgatgaggaggtctctaag

SEQ ID NO: 717 - DNA, 202 bp, Homo sapiens
ttggttgctagacgaccaagcagacaaaagttaaagattgaaaaggcatttttaaagatgccaaatcttggcggctgaattattttatctgtgtctttctactggagggcttagtattcctaccaggttaccattgcttttgtcggctagatggctggtagaagatttttttgaagtctagctcttaaggcattatctgttt

SEQ ID NO: 718 - DNA, 202 bp, Homo sapiens
ttttttttaattctttaaagcctaattttaaatcaaagtcatttgttcatttcggtaagatgtttcaggagcatgttataaatacacaatctttatagatactgccaagttagacatgtgttcacaagttcatcttgcatgtgtacacagtttgtagctgacatgcgttcctcactgagggcttccattctttctcattcat

SEQ ID NO: 719 - DNA, 202 bp, Homo sapiens
ggcttccattctttctcattcattctgtgagatgctgaatctttggctctccgctttgtggggtctgctgtgctggaattaagtcacatctatggcaaaccttcagctactttcctggttcatttccaggcagggtttcctggcatggggatcaattagggagccctgaggtgtgctcatcacagactctcttgaaaattag

SEQ ID NO: 720 - DNA, 202 bp, Homo sapiens
tcagctactttcctggttcatttccaggcagggtttcctggcatggggatcaattagggagccctgaggtgtgctcatcacagactctcttgaaaattagagatcttctgaaacctgtctggcctatatatgtaaatttgacaaaatattcctctggaaggaaatcttactaactgacttagaaattgttcaatcactcggt

SEQ ID NO: 721 - DNA, 202 bp, Homo sapiens
tcaattagggagccctgaggtgtgctcatcacagactctcttgaaaattaggatcttctgaaacctgtctggcctatatatgtaaatttgacaaaatattctctctggaaggaaatcttactaactgacttagaaattgttcaatcactcggtttaactggtaaacctacagtcctccaggacatatcagtattgtcaatat

SEQ ID NO: 722 - DNA, 202 bp, Homo sapiens
actcggtttaactggtaaacctacagtcctccaggacatatcagtattgtcaatattataatattacaaaccatgaaaataattatgcttaaacactgaggtccctgaagtgaaccagactacaaacttaataatagtcaatcgataagctaaatagaaataggaaaacagattcaggagaatatttcaatacttcctaatc

SEQ ID NO: 723 - DNA, 202 bp, Homo sapiens
tttcaatagcagaacagtgattgtttcttttttgagacggagtctcgctctgtcctccaggctggagtgcagtggcacaatctcagctcactgcaagctcctgcctcctgggttcatgccattctcctgcctcagcctcccaagtagctgtagctgggactacaggcgcccgccaccacgcctggctaattttttatatttt

SEQ ID NO: 724 - DNA, 202 bp, Homo sapiens
cagtggcacaatctcagctcactgcaagctccgcctcctgggttcatgccattctcctgcctcagcctcccaagtagctgtagctgggactacaggcgccctgccaccacgcctggctaattttttatatttttagtagagacggggtttcaccgtgttagccaggatggtctcgatctcctgaccttgtgatctgcccacc

SEQ ID NO: 725 - DNA, 202 bp, Homo sapiens
ttttttatatttttagtagagacggggtttcaccgtgttagccaggatggtctcgatctcctgaccttgtgatctgcccacctcggcctcccaaagtgctagggattacaggcatgagccacagcacccagcccagaacagtgatttttaatacaattaaacaacacaatcatccctttcttggtgatggtatgaagcattt

SEQ ID NO: 726 - DNA, 202 bp, Homo sapiens
ccagaagctccttttgaaggcttcctctttattgaactttggagaaagaacacttctcagctgaaagagaactgaagactaagaattctagaatcctctcagccaacaggtgcccaaaggaaaatctttctgcctgacacacaagtatcagattttgtatgaatattatgttttgtggaaatttatctttgggaggttgtca

SEQ ID NO: 727 - DNA, 202 bp, Homo sapiens
tccttttgaaggcttcctctttattgaactttggagaaagaacacttctcagctgaaagagaactgaagactaagaattctagaatcctctcaccaacagcgtgcccaaaggaaaatctttctgcctgacacacaagtatcagattttgtatgaatattatgttttgtggaaatttatctttgggaggttgtcatattagac

SEQ ID NO: 728 - DNA, 202 bp, Homo sapiens
cgtgagtaccctgatgcctgagaagcccttttgatggcacaggcaggtggaggccaggttgttgtaaggacactgcttttgtgcctttgttcagcctcacctcttggtgtgctttcgagtgcactaaggtactattgaggatttggtacaaggagcagagagagggctgcttgaaattcctaaaagacaatcaggtgcattc

SEQ ID NO: 729 - DNA, 202 bp, Homo sapiens
atgggaagattagttaaggaacttttatgagggaggggacaagtttgtgttttatgtactatttctggaaaattcaaggagtggcaatgtttcagtccccgttgatgtgaaaataagaaccttcgtccaatggcagctggtgctgaaatgcaactttctggaagtctcctgtttaatgggtctgtgaggttttgacctctgg

SEQ ID NO: 730 - DNA, 202 bp, Homo sapiens
gggacaagtttgtgttttatgtactatttctggaaaattcaaggagtggcaatgtttcagtccccgtgatgtgaaaataagaaccttcgtccaatggcagcgtggtgctgaaatgcaactttctggaagtctcctgtttaatgggtctgtgaggttttgacctctgggtcccaacggatcccaggacccacagtgctgttct

SEQ ID NO: 731 - DNA, 202 bp, Homo sapiens
ttttttttttttttttttttgagacggagtcccgctctattgcccaggctggagtgcagtggcgcgatctcggctcactgcaaactccacctcctggcttcgacgccgttctcctgcctcagcctcccaagtagctgtagctgggactacaggcatgtgccaccgcacccggctaattttttttgtatttttagtagagacg

SEQ ID NO: 732 - DNA, 202 bp, Homo sapiens
ctgggattacaggcgtgagccaccgcgcccggcccagttccctttctgtttctctctccttttctccgggtgtcaggaacttttgtggagataaggaaatagtgctaggggaagaggagctgaaaaagtcaaccttatttgctacaaagtgagagcaatgtggtcctgtgtcatcgcaaacctgagcgtttgttaaacctga

SEQ ID NO: 733 - DNA, 203 bp, Homo sapiens
cacggaccaaagcgcctctcctctgcagccagaaacagtgattcatttgattttaatttttaacattagcatttttctcatgactcactgataatttaatacttggaatttaaaaggttgaaacagttatttactttttaggaaattaaaacattccctaggtggatatatattttccctctgaaacagaagatgtactcgtg

SEQ ID NO: 734 - DNA, 201 bp, Homo sapiens
cagtctgtttcttcttattctttctcttatattgaattgctcagaaaactgggtgaaaatgtcaccccagcaaatacttccctactatcatttacctcctctggtcttcgttgtagcagtattaagtgagggcagtctttctcataggtattttatgaatcttgtgagctaagatcctgcccaagatcatagatctttctg

SEQ ID NO: 735 - DNA, 202 bp, Homo sapiens
ctgaatagttttactgagggtattgtaggacttctgctgaatggatgaggaaaaacctgggctggcacaaggatcttgtcataacttaaaaatacacacactacagttattctcgctaactttctgatattaaatgcagttgtaactgggaaatttgctatataataatgggttgtaataaaaaatgtgattcatccaacag

SEQ ID NO: 736 - DNA, 202 bp, Homo sapiens
aatagttttactgagggtattgtaggacttctgctgaatggatgaggaaaaacctgggctggcacaaggatcttgtcataacttaaaaatacacacatacaggttattctcgctaactttctgatattaaatgcagttgtaactgggaaatttgctatataataatgggttgtaataaaaaatgtgattcatccaacagctc

SEQ ID NO: 737 - DNA, 202 bp, Homo sapiens
aacaacaccaatgaactgatgctcagaaacttactttaatgaataggtaataattattctaaatcgggatagcaaggatatttttgtgccattgcacaatagccaagaacattctcaggagtctaggcatttccactggaaacttgcatattcattctgatgactataataaagaggatatggtagatctctgcacagagga

SEQ ID NO: 738 - DNA, 202 bp, Homo sapiens
tgcatattcattctgatgactataataaagaggatatggtagatctctgcacagaggatcctttccccatcatccttcctgtcttccagttctgttctccagggagaatgtattaggaagggaaaaagcaaacggaaagtagggtgctccagttctcagtatttgcctgcatgctcaagttagttgtgagatgtggctctca

SEQ ID NO: 739 - DNA, 202 bp, Homo sapiens
ggatatggtagatctctgcacagaggatcctttccccatcatccttcctgtcttccagttctgttctccaggagaatgtattaggaagggaaaaagcaaactggaaagtagggtgctccagttctcagtatttgcctgcatgctcaagttagttgtgagatgtggctctcagcattttatactaaaatgaagtatcatattt

SEQ ID NO: 740 - DNA, 202 bp, Homo sapiens
gtagatctctgcacagaggatcctttccccatcatccttcctgtcttccagttctgttctccaggagaatgtattaggaagggaaaaagcaaacggaaagctagggtgctccagttctcagtatttgcctgcatgctcaagttagttgtgagatgtggctctcagcattttatactaaaatgaagtatcatattttatgcat

SEQ ID NO: 741 - DNA, 202 bp, Homo sapiens
aaaaagcaaacggaaagtagggtgctccagttctcagtatttgcctgcatgctcaagttagttgtgagatgtggctctcagcattttatactaaaatgaaagtatcatattttatgcatgatatccttagaacagaactgtgcaaaacagtagccactagccacttgaggcaattaaacacttgaaatgtgactagtccaaa

SEQ ID NO: 742 - DNA, 202 bp, Homo sapiens
gcattttatactaaaatgaagtatcatattttatgcatgatatccttagaacagaactgtgcaaaacagtagccactagccacttgaggcaattaaacaccttgaaatgtgactagtccaaattgagatgtgctgtaattgttgaatacgcacctgatttcagagttagtatgaaaaaaaaaagatggaagatgacacatta

SEQ ID NO: 743 - DNA, 202 bp, Homo sapiens
atttttaaaatttattttatcatttccttttattttttaatgtagctactagaaaattttaaattacacgtatggctagtattatgtttctgttggacaggtggttagttagagcaataaattagagtggactcgcatcacttgcgttcatttccagactgatggagtttctcaggtgacagtgaaaatgatgaaggtagag

SEQ ID NO: 744 - DNA, 202 bp, Homo sapiens
gttcatttccagactgatggagtttctcaggtgacagtgaaaatgatgaaggtagagccaacacttacatagaaattcgcgaatagctataggaccccgactaaaggttaatgatgttcatctactgtgtcttagcagagaaaaagctgggagccatcgacatggtagaaagagaactggcttcacgttttggaagtctgtg

SEQ ID NO: 745 - DNA, 202 bp, Homo sapiens
atgtatcattgttcaaagtaaatatggtttataaagtgaaaattgagtaagtggaccctgtattaggtgtctattactgcaaaacaagttacctcaaactgttgtggtttaaaacaagcttttatcaactcacgtattttcgaagggttaggaattcaggagtatcttagctgggtggcactactacctcagaatctctcag

SEQ ID NO: 746 - DNA, 202 bp, Homo sapiens
agtggaccctgtattaggtgtctattactgcaaaacaagttacctcaaactgtgtggtttaaaacaagcttttatcaactcacgtattttcgaagggttaaggaattcaggagtatcttagctgggtggcactactacctcagaatctctcaggacatggcagccaaggtgtcaaccagagctaaagtcatttgaagacttg

SEQ ID NO: 747 - DNA, 202 bp, Homo sapiens
ttgcctcagtctcagaagtcaaacactgtcactttgaccttattctattaggagccactaaatccagtcaagtctcaagggaagtgggaaggggtattgacttgaaccccaccttttgaagagactgtcaaaaaatttgtgacatatatttcacctccacagacctttaccatctaaaagtcgatgacacaatttggattat

SEQ ID NO: 748 - DNA, 202 bp, Homo sapiens
tgacatatatttcacctccacagacctttaccatctaaaagtcgatgacacaatttggattattctcatcatatctaactaaatcagcatcgaggaaccaaggggaaaaaagcacttggggcacatagcacccaattcaagaattatcctcaaacccagctactgaaaggggctgctgtaaccttaacaccagttttacctc

SEQ ID NO: 749 - DNA, 202 bp, Homo sapiens
cctccacagacctttaccatctaaaagtcgatgacacaatttggattattctcatcatatctaactaaatcagcatcgaggaaccaggggaaaaaagcaccttggggcacatagcacccaattcaagaattatcctcaaacccagctactgaaaggggctgctgtaaccttaacaccagttttacctcacggctgttgaagt

SEQ ID NO: 750 - DNA, 202 bp, Homo sapiens
gaaaaaagcacttggggcacatagcacccaattcaagaattatcctcaaacccagctactgaaaggggctgctgtaaccttaacaccagttttacctcacaggctgttgaagtgacccactatgactctaagagtaattttacctaaagccatcactcactaatcagagcttgccagctcccagaaacttgctaatattaat

SEQ ID NO: 751 - DNA, 202 bp, Homo sapiens
tactgaaaggggctgctgtaaccttaacaccagttttacctcacggctgttgaagtgacccactatgactctaagagtaattttacctaaagccatcactacactaatcagagcttgccagctcccagaaacttgctaatattaatgagcattttttttcaacacaatacagaacgtttctctttttaataaaactcccaac

SEQ ID NO: 752 - DNA, 202 bp, Homo sapiens
actatgactctaagagtaattttacctaaagccatcactcactaatcagagcttgccagctcccagaaacttgctaatattaatgagcattttttttcaaacacaatacagaacgtttctctttttaataaaactcccaaccttctctttgttctttggacacaacagagaccaccatggtctatgtgtatgccccaaattg

SEQ ID NO: 753 - DNA, 202 bp, Homo sapiens
gttgacaaccacatttgaaaacaggaaaaaaaaactgtttctataagggattgagctcatggcctagttgtcttcagagtagaacaaacttttggttctgcttggagtctctgaataacaaaatctccaattgaacttcacattcattagaactaacaatttgaatactttgcccatcattaaccataattcattttttgag

SEQ ID NO: 754 - DNA, 202 bp, Homo sapiens
gactgtggttcttgagcctttcgcctaccactgccccttcttcatacccttgagcagcctttctttttttaatcaatggagattttaagaagaggagactagctttcctagatttcagaatacatgtggaagcattttatatttccctgctctactgtcttagtgtatagggaattttactggtatctggattaaattaagt

SEQ ID NO: 755 - DNA, 202 bp, Homo sapiens
gcctaccactgccccttcttcatacccttgagcagcctttctttttttaatcaatggagattttaagaagaggagactgctttcctagatttcagaatacagtgtggaagcattttatatttccctgctctactgtcttagtgtatagggaattttactggtatctggattaaattaagtccttggtcttccttatcccctc

SEQ ID NO: 756 - DNA, 202 bp, Homo sapiens
cctctttgtcctaaagctgtctccctagctagagaacatttattttgggaaagcctttatgatattaaaactcatcctatagtacctgtcttatttgcagagtggacaaaacatttcccttggccctttgtggaaggttgactattggatgtctgtttctttcctagaaattgtggtttttttctccttttttgagttttga

SEQ ID NO: 757 - DNA, 202 bp, Homo sapiens
tatcactctgctattctaaatctgctggaaagttatttaaggcatatgaaacaagggtgaaatgagatgtaatgaatttttttaaaaggtaccatgactgcttcgtggagaatagattgaataggaaaggagaaatggagaatgattaggaagctgcagtaggaataattttgagctggccctcaagatttctgctctctga

SEQ ID NO: 758 - DNA, 202 bp, Homo sapiens
tggagaatagattgaataggaaaggagaaatggagaatgattaggaagctgcagtaggaataattttgagctggccctcaagatttctgctctctgatgtattgcactctgcagaacccttgcctttgagagcagctgacctgtaaatacgatgggaggcaatcagttgactctgagttaatcagaaggagattatcctgtg

SEQ ID NO: 759 - DNA, 202 bp, Homo sapiens
taggaataattttgagctggccctcaagatttctgctctctgatgtatgcactctgcagaacccttgcctttgagagcagctgacctgtaaatacgatggagaggcaatcagttgactctgagttaatcagaaggagattatcctgtgtagcctcacctcaccaggtgagatatttaaaagaaagtgaacaggcagagagag

SEQ ID NO: 760 - DNA, 202 bp, Homo sapiens
ttatcctgtgtagcctcacctcaccaggtgagatatttaaaagaaagtgaacaggcagagagagtactcctactggcctgggagaaagcaaagagccacactggtgaactacccatgtgggccatgtggcaaggaaccgtgagtagcctctgggaactgagactggtccctagcaaggagctaccaaaggaagcagggccct

SEQ ID NO: 761 - DNA, 202 bp, Homo sapiens
ctggcctgggagaaagcaaagagccacacggtgaactacccatgtgggccatgtggcaaggaaccgtgagtagcctctgggaactgagactggtccctagacaaggagctaccaaaggaagcagggccctcagccacaaggaaataaatgttaacaaggaagaggaccttggtcctcagataaagctgagagccctggtcaa

SEQ ID NO: 762 - DNA, 202 bp, Homo sapiens
ggccatgtggcaaggaaccgtgagtagcctctgggaactgagactggtccctagcaaggagctaccaaaggaagcagggccctcagccacaaggaaataaagtgttaacaaggaagaggaccttggtcctcagataaagctgagagccctggtcaacaccttcatttcagcctgtgagaccctgagcagaagtaccagctgc

SEQ ID NO: 763 - DNA, 202 bp, Homo sapiens
ccctagcaaggagctaccaaaggaagcagggccctcagccacaaggaaataaatgttaacaaggaagaggaccttggtcctcagataaagctgagagcccctggtcaacaccttcatttcagcctgtgagaccctgagcagaagtaccagctgccacacctagacttctgtccatggaaactgtgagataacaaatttatgt

SEQ ID NO: 764 - DNA, 202 bp, Homo sapiens
gaccttggtcctcagataaagctgagagccctggtcaacaccttcatttcagcctgtgagaccctgagcagaagtaccagctgccacacctagacttctgatccatggaaactgtgagataacaaatttatgttattttggcgaggtgcagtgggtcatgactataatcccagcactttgggaggccgaggtgggtggatca

SEQ ID NO: 765 - DNA, 202 bp, Homo sapiens
cgaggtgggtggatcacttgaggtcaggagctcgagaccagcctggccaacatggcaaaaatgcgtctccactaaaaatacaaaaaaaaattagctgggcagtggtgatgcacacctatagtcccagctactcgggagactgatgcagaagaattgcttgaacctgggaggcagaggttgcagcgagccaagattgctccac

SEQ ID NO: 766 - DNA, 202 bp, Homo sapiens
ggtgggtggatcacttgaggtcaggagctcgagaccagcctggccaacatggcaaaaatgcgtctccactaaaaatacaaaaaaaaattagctgggcgtggttgatgcacacctatagtcccagctactcgggagactgatgcagaagaattgcttgaacctgggaggcagaggttgcagcgagccaagattgctccactgc

SEQ ID NO: 767 - DNA, 202 bp, Homo sapiens
ccatctcaaaaaaaaaaaaaaaattatgttattatattttaagtcactacacttgtggtaaattgttacttaagaaataactaatgcataagctatttgcaggcattccagcttcaaaaagatggtgcttgtattagatttgtggtaaggaagatggagagaaatgagtggatttgagatcttcttcagaggtatatttgac

SEQ ID NO: 768 - DNA, 202 bp, Homo sapiens
taagattttttaaatttgagctacttagtgactagtgatcacaatgaatgttatcacagtttcagctatcataatttcaggaggggaagcatggaggaagagacgtgatgctagggaaacatcatgagttcagcttggacatgttgaacttgagatgtttgtgaaatatgcaaacggtattgtcaagtaagcagtgggtatg

SEQ ID NO: 769 - DNA, 202 bp, Homo sapiens
gttcagcttggacatgttgaacttgagatgtttgtgaaatatgcaaacggtattgtcaagtaagcagtgggtatgtgagatgaactcagaggagactcttatggttggaaatggaaatttggaagttatcagcatatcagtaccaaagccatggaaatggaataggaaagaaaaaggaaaaggggcttcgaccaaacactga

SEQ ID NO: 770 - DNA, 202 bp, Homo sapiens
gaggaagtagaagtgaaaaaaggctgaaaagaggtggcctgaagagcacagggaaacctagagagtgtggtaccatgaaaaccacgggaggacattgttcagtaaaggaggacagagtcacaatattgagtgctactgacatctccaggaagaacaggggtttgtgaccttgatttaagtgagccattagagctttgagcag

SEQ ID NO: 771 - DNA, 202 bp, Homo sapiens
gtggaagtacaagtaggcaatttttctttttaatttttaatcttgaagtcagtagagattcatgtgtacctttcatacaatttcccccaatggttatatccttgtatcattatagtacaatagcaaacactagaatttgacagtggtacaattgtgcctatttccatggcattaatcacgtgtttagatctgtataagcatc

SEQ ID NO: 772 - DNA, 203 bp, Homo sapiens
gtagagattcatgtgtacctttcatacaatttcccccaatggttatatcttgtatcattatagtacaatagcaaacactagaatttgacagtggtacaatcgtgtgcctatttccatggcattaatcacgtgtttagatctgtataagcatcactgcaatcaaggtattccattatcacagagatctattttatgcttttgtg

SEQ ID NO: 773 - DNA, 202 bp, Homo sapiens
catttagaaaatattatataaatgaaatcatacagtatatgaccttttcagatgggctttttttttttttcactcagcgtaacgcccttgaaatgtatccagggttgttgcacgtatcagtagttcattccttttattctatgatatggatgtatcatagtttattcagtcattcaccttgtgaagagcatctatctgatct

SEQ ID NO: 774 - DNA, 202 bp, Homo sapiens
atgttctaattggattgcttatttttaacgagttttaagacttctttatataatctagatatgaatctattctcagatatgtgatttgcaaatattttctcttctcagtctgtagcttgtcttttttcatcttaataggatcttttacaaatctttttcattttttttatttttgatgagatcaaattatcaatttttcatt

SEQ ID NO: 775 - DNA, 202 bp, Homo sapiens
acttctttatataatctagatatgaatctattctcagatatgtgatttgcaaatattttctctctcagtctgtagcttgtcttttttcatcttaataggactcttttacaaatctttttcattttttttatttttgatgagatcaaattatcaatttttcatttatggattctgcttttggtgtcatgtctaacaactcttt

SEQ ID NO: 776 - DNA, 202 bp, Homo sapiens
ataatctagatatgaatctattctcagatatgtgatttgcaaatattttctctctcagtctgtagcttgtcttttttcatcttaataggatcttttacaaagtctttttcattttttttatttttgatgagatcaaattatcaatttttcatttatggattctgcttttggtgtcatgtctaacaactctttgccaggttct

SEQ ID NO: 777 - DNA, 201 bp, Homo sapiens
tatgaatctattctcagatatgtgatttgcaaatattttctctctcagtctgtagcttgtcttttttcatcttaataggatcttttacaaatctttttcatttttttttatttttgatgagatcaaattatcaatttttcatttatggattctgcttttggtgtcatgtctaacaactctttgccaggttctaggtcttga

SEQ ID NO: 778 - DNA, 202 bp, Homo sapiens
ttttctatgtttttcctaaaaattttattaatttacatttaaatctatgatcctgatccattttgagttaatatctatataacatgtaaggtttaggggtcttttttgcctatggatatccaattactccagcaccattggttggaaaaaacaggttcttccttcattgaattttaccatcgtcaaaaatcagtttttcata

SEQ ID NO: 779 - DNA, 202 bp, Homo sapiens
aattatagctatgtaagtcttgaaataaggtacagtgattcccctcactttcttcttttatagaatagttttagttattccagttcttttgcttttccatattacatttcagaataatcttacctgtatctacaaaaataccatactaggtttttgttggaattctgttaaacctgcatatcagtctggggagaactgatat

SEQ ID NO: 780 - DNA, 202 bp, Homo sapiens
tattccagttcttttgcttttccatatacatttcagaataatcttacctgtatctacaaaaataccatactaggtttttgttggaattctgttaaacctgctatatcagtctggggagaactgatatttttactccattgagtgtttgaatacatgaacactttgtctctccatttatttctaatttttaaatttctttcat

SEQ ID NO: 781 - DNA, 202 bp, Homo sapiens
taccatactaggtttttgttggaattctgttaaacctgcatatcagtctggggagaactgatatttttactccattgagtgtttgaatacatgaacacttctgtctctccatttatttctaatttttaaatttctttcattagcattttgtagtcttcagtatcagagtcctgtccatattttttcttagatttgcaactaa

SEQ ID NO: 782 - DNA, 202 bp, Homo sapiens
gatttgcaactaagtattttatctctctcttttaatggtttttacatagtattgtattttaaatttgtttcaacgtgttaattgctaatatatagaaatacgaattgatttttggatgttgaccttgtatcttgtgaccttgttgaatttatttatcaggtctagaagtgattttgtagattttatggaattttctatttac

SEQ ID NO: 783 - DNA, 202 bp, Homo sapiens
ctgagtataggattcttagatgattattattttctcaaagaatttcaaaaatattgtgccacttccttctggcttccatggtttctgatgagaaatacacgtgtgatttgaattgttttacccctctctggctgctttcaagattttctttgtccttagttttcagaagtttggccacggtgtgtcttggaatttgtttctt

SEQ ID NO: 784 - DNA, 202 bp, Homo sapiens
ggtgtgtcttggaatttgtttctttgctctttttttgttggtttgtttgaggttcattcatattcttgagtctataggctatttttttccaccatatttaagaaaatcttttgccatttcatttttgatgctttttaagcctcatctttttttttttttctcctttccttctgggactctgatgacaagatcttttgtgata

SEQ ID NO: 785 - DNA, 202 bp, Homo sapiens
ctttttttgttggtttgtttgaggttcattcatattcttgagtctataggctatttttttccaccatatttagaaaatcttttgccatttcatttttgatagctttttaagcctcatctttttttttttttctcctttccttctgggactctgatgacaagatcttttgtgatagtcccacatatacctgaaactgttcact

SEQ ID NO: 786 - DNA, 202 bp, Homo sapiens
tgttttattctagttcacggattctttcctcggtcttcttcattccgttgttgagcccatcattgagttatttatgtcaattattttgatttttagttctagaaatttccatttgattcttctttagatcttctattcttttgctgacatttcctgttctttcatttatttcaagcctgggtgagactgctcatggaagcaa

SEQ ID NO: 787 - DNA, 202 bp, Homo sapiens
atggaagcaaatttatgctgtctgctttaaaattcttctcatcttagggcagcatctgatgattgccttttatccattagtttgagttcttcctggttctgtggtataaccagtgatttgttattgaaacctggacagtttggattataggattttagatcttatttaaatcttgtgttttaggaggcctgctggatgaagg

SEQ ID NO: 788 - DNA, 202 bp, Homo sapiens
agtgatttgttattgaaacctggacagtttggattataggattttagatcttatttaaatcttgtgttttaggaggcctgctggatgaagggggagaagtagccaccttcactctgccaggtggggataggagtccaggttttccactctgcctccactgacaggggatgaggggctctttgttactgctatgtggggatag

SEQ ID NO: 789 - DNA, 202 bp, Homo sapiens
tagggggtatcattatcccaagagagaaggtgtggagtatctattcctgccgtgaagcctgggctctcacttggcttttgctatcagaggtgtggataggatctgaagtttctttctgtggtatttggctgagtagagtggttattatctaaatgcttcgtgtcttgctagactattccttacttcgtcttttggccagagt

SEQ ID NO: 790 - DNA, 202 bp, Homo sapiens
ccactctgggatgtatgaagtaaaaagaaacaccagggaacttaccacctgtgttcttcaagtcctaatgtccctagctagcctgcctttctctctctcccttttcagatattcttaatttatttaatatataatgcccagggtttttagttgaacttagaggaatagggaaaagtatgtctacttcatcttcctggaaaca

SEQ ID NO: 791 - DNA, 202 bp, Homo sapiens
ctcagcagatagtttttatttgttcttcaaatgtccttataaatctgcagagtaaaaggtgataaatgatttcaattttccaaagggaaaaattgagacacgttaaaggtaatttacctgatcaaagttacctagaaatacagtcttagcttcaagtaaaacctcacaattttaaatagctattattttgcttttctttttt

SEQ ID NO: 792 - DNA, 202 bp, Homo sapiens
acctagaaatacagtcttagcttcaagtaaaacctcacaattttaaatagctattattttgcttttctttttttctctctctgctaaaaggaaaaaaattagctgattggtagagggaaaggaagaaatggttacccacaaaaagactaaaaggactatttttttggcttggaagcagtaactgtttaaaattactaaagga

SEQ ID NO: 793 - DNA, 202 bp, Homo sapiens
gtgattacataatcttctcagttactcctttatatcttatcttataaatgaatgactttaattatttatactatgtcctgaaacttaagtgtcaaggtgcatcatttcataatcattgtcactctcttgattaaagactcacacaaaaacaggaaaaaggatgaaactgtttcattactatattaggccattcttgtgttgc

SEQ ID NO: 794 - DNA, 202 bp, Homo sapiens
ttgtgttgctataaatacctgaggctggataattcataaagaaaagaggtttaattagctgatgggtctgcaaacagtgcaagaaataaggtgccagcatactgctactggtgacagcctcaggaaggttacaatcatggtggaaggtgaaggaggagcagccatctcacatggtgagaggggaagcaagagagcaaggggg

SEQ ID NO: 795 - DNA, 202 bp, Homo sapiens
tataaatacctgaggctggataattcataaagaaaagaggtttaattagctgatgggtctgcaaacagtgcaagaaataaggtgccagcatctgctactgagtgacagcctcaggaaggttacaatcatggtggaaggtgaaggaggagcagccatctcacatggtgagaggggaagcaagagagcaagggggcagtgccac

SEQ ID NO: 796 - DNA, 202 bp, Homo sapiens
ccaaacacctcccaccagactccctgcaacattgtggattattttttaacatgatatttgcaggggacaaacatcctaactataccaattactttgcaggagcgttcatctttcatcagaaccccttgggtcctaggatttctgtaggttgcgtagcatttcaagaaagattttagactgctcccttaaaacccattatctg

SEQ ID NO: 797 - DNA, 202 bp, Homo sapiens
atcatttagcaccacactttaaaatttactgagtctaaaatccaaagactggggaagatgggaaagacctttcagggacaagtgaactggcctacagttcagctcctattatggccattttgattcacctagctcatatgagtttagatgtggcatatattgcaggaataagggtggcaaatggcatgttttcttcaaaaga

SEQ ID NO: 798 - DNA, 202 bp, Homo sapiens
accactcaatgtaactttctgccatgaacataaccagccacacataaactgtctgcagaaaaggaagttccatcctataagcttggcaggagggtatgtaagggtcaacaaagaaatctctgctactcgtttgcagtcatttccatgaggctaattgtcacctccagaaatcagcccagaaggaataaatgcattttcccct

SEQ ID NO: 799 - DNA, 202 bp, Homo sapiens
atcaaaactactgctgactgcagcaaagagtaaggaacacttagagagatggagcaatatttcaaaaatacttcttagaaaagaattggaagtctttgcaatttctaaatcctacgcttttatgttttcaaatgaatcagatgatattgtgtcaacaaaagcatatcttatttcaacacttatttctatatctcagttggaa

SEQ ID NO: 800 - DNA, 202 bp, Homo sapiens
ctgactgcagcaaagagtaaggaacacttagagagatggagcaatatttcaaaaatacttcttagaaaagaattggaagtctttgcatttctaaatcctactgcttttatgttttcaaatgaatcagatgatattgtgtcaacaaaagcatatcttatttcaacacttatttctatatctcagttggaatttcgatactctt

SEQ ID NO: 801 - DNA, 202 bp, Homo sapiens
gactgcagcaaagagtaaggaacacttagagagatggagcaatatttcaaaaatacttcttagaaaagaattggaagtctttgcatttctaaatcctacgctttttatgttttcaaatgaatcagatgatattgtgtcaacaaaagcatatcttatttcaacacttatttctatatctcagttggaatttcgatactctttc

SEQ ID NO: 802 - DNA, 202 bp, Homo sapiens
aaagcatatcttatttcaacacttatttctatatctcagttggaatttcgatactctttctgtcattttcattttcttctcatgccatgtaaccttgtccctgtttgagaaggcatgctaaaaagtttgcaaagaataatgcagtttacttctatgttcaaacattcagtactctttctatataatacttctaattgcaaaa

SEQ ID NO: 803 - DNA, 203 bp, Homo sapiens
taaccttgtcccgtttgagaaggcatgctaaaaagtttgcaaagaataatgcagtttacttctatgttcaaacattcagtactctttctatataatacttacgtaattgcaaaattttgtgcacctagaaagaggatctctctatctaaatattgatgtcttaataagttcattgtactgatgtcttactaataatctcaagg

SEQ ID NO: 804 - DNA, 202 bp, Homo sapiens
agttcattgtactgatgtcttactaataatctcaaggagagaatttatttgtaatgctgcaatttcaaagtgcaggtgttgtatcttttattcatgtttaagctttcagaatccactgagtgttttaaatggtaatgaaaaggctcaaaggttgatagctgtgcttggagagggatagtgaattagaaagatctgctacctg

SEQ ID NO: 805 - DNA, 202 bp, Homo sapiens
atctcaaggagagaatttatttgtaatgctgcaatttcaaagtgcaggtgttgtatcttttattcatgtttagctttcagaatccactgagtgttttaaactggtaatgaaaaggctcaaaggttgatagctgtgcttggagagggatagtgaattagaaagatctgctacctgactatgcattcattttccaattttttgg

SEQ ID NO: 806 - DNA, 202 bp, Homo sapiens
tctcaaggagagaatttatttgtaatgctgcaatttcaaagtgcaggtgttgtatcttttattcatgtttagctttcagaatccactgagtgttttaaataggtaatgaaaaggctcaaaggttgatagctgtgcttggagagggatagtgaattagaaagatctgctacctgactatgcattcattttccaattttttgga

SEQ ID NO: 807 - DNA, 202 bp, Homo sapiens
atccctttattcctctgtctggtgtgctgcccagggatgattccttaaactgctgcagtctgatgataaaggtgaggatggacagaggcaagggagacacagggaacacaaagcagaagcctgattgaactgtacttgattcccactctctctctggaccttgtcagttacagaggccaatacatccctaatcaccccaact

SEQ ID NO: 808 - DNA, 202 bp, Homo sapiens
gatgcacttgcataggctgcttcctagatacttggtggaaaggcccatatgtcaaaatacaacacacaccaaagtgggtacatgtgatcagatcaggcaactctgtcagcctagggaccatccttaccaccgcctgcaaatgaggataaatgaggtgcagttttggaaaatctctgggcttgggaccaatttgggcagcaag

SEQ ID NO: 809 - DNA, 202 bp, Homo sapiens
taggctgcttcctagatacttggtggaaaggcccatatgtcaaaatacaacacacaccaaagtgggtacatgtgatcagatcaggcaacctgtcagcctacgggaccatccttaccaccgcctgcaaatgaggataaatgaggtgcagttttggaaaatctctgggcttgggaccaatttgggcagcaaggtggaggagtga

SEQ ID NO: 810 - DNA, 202 bp, Homo sapiens
tgatcagatcaggcaacctgtcagcctagggaccatccttaccaccgcctgcaaatgaggataaatgaggtgcagttttggaaaatctctgggcttgggactcaatttgggcagcaaggtggaggagtgaaacagcatattttgctgatagagaagagatagtggtgagtcatacagatacacagatgcttggagttgttgt

SEQ ID NO: 811 - DNA, 201 bp, Homo sapiens
gagaagggataagcccttggatgactgaaatgggatttatgcaaggtggccttaggcagaatcagtactccctgcctgcctcttgccaccctcacttacttctcactttgtttcttccaacaccccaagcaagtcagtagttctctgcggccttctgttcagtgcccactcttttctcatcttttctccttccctatcttt

SEQ ID NO: 812 - DNA, 202 bp, Homo sapiens
cctgcctgcctcttgccaccctcacttactctcactttgtttcttccaacaccccaagcaagtcagtagttctctgcggccttctgttcagtgcccactccttttctcatcttttctccttccctatcttttctccttcccctcccacctcatcccactgcactttcccatcagtctcacgtgatggggtttgtgaggaaat

SEQ ID NO: 813 - DNA, 202 bp, Homo sapiens
gtttcttccaacaccccaagcaagtcagtagttctctgcggccttctgttcagtgcccactcttttctcatcttttctccttccctatcttttctccttcctcctcccacctcatcccactgcactttcccatcagtctcacgtgatggggtttgtgaggaaataatgggtactaatatttgcttcaagaaggagttttctc

SEQ ID NO: 814 - DNA, 202 bp, Homo sapiens
ccttcccctcccacctcatcccactgcactttcccatcagtctcacgtgatggggtttgtgaggaaataatgggtactaatatttgcttcaagaaggagtctttctcctcctcctcctccttttcctactcctcctcctcttccttctcctcctcctctcctcctactcttcttcttctccgttctccttctccttctcctc

SEQ ID NO: 815 - DNA, 202 bp, Homo sapiens
tggagatggggtttcaccatgttggccaggctggtctcaaactcctgacctcaagtgatctgctggcctcggcctcccaaagcgctaggattacaggcatagagccacctcgcccagccaagaaggagtttcttatgtgacttatcctagcactgacctaataaaggggaaagagatctggtgtggactcccatcttccatg

SEQ ID NO: 816 - DNA, 202 bp, Homo sapiens
gccatcacacaggaccatcccggagatataaaataattctaactactaccatttctggagcacttatgtatcatactctgcatactatctacttgaaatgctcacatttgttttaatccttttagctgtcaagagaatacatgattttatatacattttccagttgaggagcttgaggtctagaatggttaaaaattcttgc

SEQ ID NO: 817 - DNA, 202 bp, Homo sapiens
ctatctacttgaaatgccacatttgttttaatccttttagctgtcaagagaatacatgattttatatacattttccagttgaggagcttgaggtctagaaatggttaaaaattcttgcccagccgggtgtggtggcttacgcctgtaatcccagcattttgggaggctgaggcaggtggatcacctgaggtcgggagtttga

SEQ ID NO: 818 - DNA, 202 bp, Homo sapiens
ttgggaggctgaggcaggtggatcacctgaggtcgggagtttgaagccagcctgaccaaagtggagaaatcccatctctactaaaaatacaaaattagccacggcattgtggtgcatgcctgtaatcccagctactcgggaggctgaggcaggagaatagcttgaaaccaggaggcggaggttgcgatgagtcaagatcgtg

SEQ ID NO: 819 - DNA, 202 bp, Homo sapiens
caaatatatgcaattttattttagaggcaaattcttccagatagaaggagtgaggaacagggtaaatcctgtcaacaaaaatgattaaaattgtggaaaactaatcatttcagaattcttaaatccaccaaaggtataaaatacatatagaagcatttgttcaaaaaatcctgccaaaccttggttaagaactatgaaaatc

SEQ ID NO: 820 - DNA, 202 bp, Homo sapiens
aagcctcatccataaatgttgtggaaaagaatatgatctcatggcaaacaaataagaaaggctatcatgacaactagcttgaagctgtaattctgataagagcataataataataataataacagagagagacagggaatgatatggctgtagtgctctttgataagctccaagatatctctaggcttctgaaaaggtaagt

SEQ ID NO: 821 - DNA, 202 bp, Homo sapiens
gtgttgagtacaacctctgaccaataactgactgatgactagagtatgctaacacaaggaaaatctctaggaagccagactcaacgagtaagagcaagaaacaacaacaaaaatctaagcaaagacattagaggctatgtaccatgggggaggcagactccacagaattagttcaggtaaattattaaacaaaccaagagaa

SEQ ID NO: 822 - DNA, 202 bp, Homo sapiens
ttcaacaaaaccattgcaagacatacaaagaaaaaatgtaacctatgtttaaaaaaataagtcactacctaccatttttgaggaatccagttattggactctggccaataaagacttcaaagcagatattaaatgtaacatttaaggaacaaaagaatagtataagacaatgactaatcaaatagagcatcttgataaggag

SEQ ID NO: 823 - DNA, 202 bp, Homo sapiens
aatgtaacctatgtttaaaaaaataagtcactacctaccatttttgaggaatccagttattggactcggccaataaagacttcaaagcagatattaaatgctaacatttaaggaacaaaagaatagtataagacaatgactaatcaaatagagcatcttgataaggagatagaaattatacaaaataggccgggcgtggtgg

SEQ ID NO: 824 - DNA, 202 bp, Homo sapiens
gccgggcgtggtggctcacgcctgtaatcccaacactttgggaggtcaagacaggaggataacttgaggttgggaggagaccagcctgcccaacatggcaagaaccctgtctctactaaaaatacaaaaattatctgggcgtggtggcacacacctgtaatcccagctacttgggaggctgaggcaggagaatcgcttgaac

SEQ ID NO: 825 - DNA, 202 bp, Homo sapiens
agtgaaaatttattataggagctgaacagtagatttggggtcacggaagaaaaatcgataaacttgaagatagataaatagaaattgttcaaattaaagaagcagaaagaaaaaataaagacaaatgagaaaagcctcagaaactatctccaggatagtctgtcaatcatatggagtactatcaatcatgccaactatcaat

SEQ ID NO: 826 - DNA, 202 bp, Homo sapiens
caaaagataaatataccaataaatgtgtaagactttaagtatatatacacatttctgcttttttctcttaactggcttaaaatatataagattgcataaaaccaatacctatgaaactgcatttctgggcttttaaactataaagttgtaatatatataaataatagtgcaacattttaatttcttattttttcatatatat

SEQ ID NO: 827 - DNA, 202 bp, Homo sapiens
acaagctaaattatatatgaaaatgcaaaggacccaaaatagccagaacaactttgggggaaaaaggttgagagatatatatttttcaattataaacttactggtaactgcagtaatcaggacagcatggcagtgtaataagggtagacatagattaaaggagtggcattgaaagtaaaaaaattaagtctcatatttatgg

SEQ ID NO: 828 - DNA, 202 bp, Homo sapiens
ttaaaggagtggcattgaaagtaaaaaaattaagtctcatatttatggtaagtttatttttaacaaagttactctaataattcaatgggggaaaggattgatgttttcaacaaatggtactgggagaattgtctatccatttgcaaaaagaataattcagactcctacctcatatcatgcataaaattaacttaaagtttat

SEQ ID NO: 829 - DNA, 202 bp, Homo sapiens
tgggcttcatttaaattaaaacctttagtacttcaaaaaaaaacaccattaagaaatgaaaagataaaccacaagctgggtaaaaatatttgcaattcatagtatctgataggggactaacatccataatatcaaaaaaacttttacaagtcaatatcaagaaaagcaacaacctaattaaataatgaacaagccaagacgg

SEQ ID NO: 830 - DNA, 202 bp, Homo sapiens
tgaggtcaggaatttgagaccagcctgcccaacatggcaaaaccccgtctctactaaaaataaaaataaaaaaataaaaataaaattagctgggcgtggtaggtgggtgcctgtaatcccagctactcaggagtctgaggcaggagaatcacttgaagtcgggaggcggaggttgcagtgagcctagatcacgccactgcat

SEQ ID NO: 831 - DNA, 202 bp, Homo sapiens
gtagaacacacataatatacaatttatcatcttaaccattttaaagtgtatagttcagtggtattaaatacattcataacgtgcaaccattaccgccatcctgtttccataactcttttcatcttgtaaaactgaaactctatattcattaaacaataactccccatatcccctaccccagcccctagcaatcaccattctc

SEQ ID NO: 832 - DNA, 202 bp, Homo sapiens
tcatatatgtggaatcatacagcaattatctttttattactgaattatataatttagcataatgacctcaagtttcaaacgtattgtagcatatgtcagaagtttccatttttaaaatggctgaataatatttcattgtgggtatatacctccttttgtgtatccattcatccacaggtggacagttatattgcttccacat

SEQ ID NO: 833 - DNA, 202 bp, Homo sapiens
ctttcaattattttcggtagatagccagaattgcattttctggatcatatagtaactctgttttttgtttttgagacggagtctcgctctgtcgcccaggactggagtgcagtggcgcgatctcggctcactgcaagctccgcctcccgggttcacgccattctcctgcctcagcctcccgagtagctgggactacaggcgc

SEQ ID NO: 834 - DNA, 202 bp, Homo sapiens
agtagctgggactacaggcgcccgccaccacgcccggctaattttttttgtattttcagtggagacggggtttcaccgtgttagccaggatgctttcgatcttcccgacctcgtgatccggccgcctcggcctcccaaagtgcttggattacaggcgtgagccaccacgcccggcccagtaactctgttttttaataaggaa

SEQ ID NO: 835 - DNA, 202 bp, Homo sapiens
ggtttcaccgtgttagccaggatgctttcgatctcccgacctcgtgatccggccgcctcggcctcccaaagtgcttggattacaggcgtgagccaccacgctccggcccagtaactctgttttttaataaggaagtcccattcggttttttgcagtagatgcaccattttacattcccaccaacagtgtgcagggttccaat

SEQ ID NO: 836 - DNA, 202 bp, Homo sapiens
caaaagtagaaattatccaagtgcccatcaactattgaacgaataaaatgtagtatattcatacaatggaatattattcaccaataaaataaactgctgactacatgctacaatatggatgaattaaaagaaaaacattatattaaatgaaagaaatcagaggcaaaagggcacatatatgtgattcttttcatatgaaacc

SEQ ID NO: 837 - DNA, 202 bp, Homo sapiens
ggggggaatagaattgtcctaacactggattgtggttatggctgcacaactctgtaatggaaatgcactgaaactttctgaatttcttaaagtgagaaaaagtatagtacctaaggtagacctcaagaaaactcttaaaattttttaaaagctctaggtaaattcaaccaatcaaaaaacaggcaaatcaaatacaaaaaaa

SEQ ID NO: 838 - DNA, 202 bp, Homo sapiens
tgggttctttgggtaaggatttctcattgtgaatataaagggattcttatcattaccaaaacattctgaaaacaatgctgttcctggcgtttaacccaatgttcgtatctgcacacaagataactatgcactgaaatcttactatctaatgttttcaaggtttcaaaaagcaagagagtggtaaataagggggatattgcaa

SEQ ID NO: 839 - DNA, 202 bp, Homo sapiens
atctaatgttttcaaggtttcaaaaagcaagagagtggtaaataagggggatattgcaaatggaatttctctgcagcctcacaattgagcttctgaaaacatccataaaaaaacactaagtgaattaagcctataaaatctgcattgtccccactttttttcctccttgattcctcaaagtcctcaggcttaaagaccccag

SEQ ID NO: 840 - DNA, 202 bp, Homo sapiens
catgaaaagagtaatatgtgtttatagagtatatatatatgtatgcgtgtggttaaaaaacttccaacagtacagaaagtaaaaaaagaaaaaaggaaaactttctatctctttctctctttccaaaggcaaaatggataaaaagtcttaggagtcaccatgatttctccaacactggcctaaggagcaccgtcattaatgt

SEQ ID NO: 841 - DNA, 202 bp, Homo sapiens
ctctctttccaaaggcaaaatggataaaaagtcttaggagtcaccatgatttctccaacactggcctaaggagcaccgtcattaatgtctgcctggatggacgaaataagcctcaatttaggaaaatctctaggcttgaaatctattagggaactcctctgtaccttgcttttgtaatttaataaaaatatagcttagaagg

SEQ ID NO: 842 - DNA, 202 bp, Homo sapiens
tttacaggctagttggacaattaaggatgaaacacaatgaataagaataacagaagtaatgattgccagaagcaaggcagaggagagaagttggcttaatacaaataatccaactgtgactttgcatcccacagggccctgtgcttagaaatgctcctgtttgctttcctgctccagtgtcattgtcttgaaattcttaaca

SEQ ID NO: 843 - DNA, 202 bp, Homo sapiens
agcaatgacttagtaaccacattttacagatgatttttgtaatgtttttcctaccatgctttgtttttaccctccaaatggacacactcttgggctgatgcttttctacctggaatttgttcagggaggtattttcttgacaagaagaaacatcattaggatgataaatatgaagggagataatactatacacagctttcca

SEQ ID NO: 844 - DNA, 202 bp, Homo sapiens
ctgcccctcattggtttgctaatatatggctatttagctgtagttgacagcctctgaaatttattatttttatataaagcagatattctttttttaatttctatttatttttattttttatttatttatttttttgagatggagcctcactctgtcacccaggctaaagtgcactgacacgatcttggctcactgcaacctc

SEQ ID NO: 845 - DNA, 202 bp, Homo sapiens
tatttttatataaagcagatattctttttttaatttcatttatttttattttttatttatttatttttttgagatggagcctcactctgtcacccaggctagaagtgcactgacacgatcttggctcactgcaacctccacctcccgggttcaagtgattctcctgcctcagcctcccaagtagctggggttacagactcct

SEQ ID NO: 846 - DNA, 202 bp, Homo sapiens
tctcctgcctcagcctcccaagtagctggggttacagactcctgccaccatgcctggctaatttttgtattttttagtagagacaggtttccccttgttgcgccaggctgatcttgaactcttgggctcaagtgatctgcccaccttggcgtcccaaagtgttaggattacagacgtgagccaccgtgccaggccagcaaag

SEQ ID NO: 847 - DNA, 202 bp, Homo sapiens
ttagtagagacaggtttccccttgttgcccaggctgatcttgaactcttgggctcaagtgatctgcccaccttggcgtcccaaagtgttaggattacagactgtgagccaccgtgccaggccagcaaagtaaagattctgaagttttgctacattaactaattttcatctatttcccagggagaaagaattattttcacttt

SEQ ID NO: 848 - DNA, 202 bp, Homo sapiens
cattaactaattttcatctatttcccagggagaaagaattattttcacttttatacgctaaagctgggagaattccactgtgaacacagcgcacaccaacgtatgccccaggggagtgttgccttgtgtattacatatttcacaccataacaatgttatccgattttataattaacattaaacttttataggaaaaagtacc

SEQ ID NO: 849 - DNA, 202 bp, Homo sapiens
tttctcacaccacttttttattctttcttcttcaacatccccacctcctctccaaccctgataataactgtttaaacattcatactcacttgcctactgcagttgagttatatgattagtttgtacccttttagtgccatcgatgatagggactcattattgagtcaatgtttctaaactatggctctctgacacaaatcaa

SEQ ID NO: 850 - DNA, 202 bp, Homo sapiens
ccttcatgcagacaccgggaaaccatctgtgtcctagcattttcctaggggtgaaaagggaacgggtgggtgaggctaattgtgtcttcttatctttctgagggtttacttactttttaaaatagtaagcattcaaatgttaatttatgttaattgattgcttgttcatgaggattaagagaatttgtggagtctcattgga

SEQ ID NO: 851 - DNA, 202 bp, Homo sapiens
ctttttaaaatagtaagcattcaaatgttaatttatgttaattgattgcttgttcatgaggattaagagaatttgtggagtctcattggatggagggtaaacgttttaaagtcagcacctgagttaaatttgatgcaaatgtatatttctggcattaacttgaatttctgtaatatttctattcagagagttgagacatttt

SEQ ID NO: 852 - DNA, 202 bp, Homo sapiens
tcaaactccaatcagcctatgaatcccctggtgaatttttttaaatatggatttggtaggtttgagataggacctcagattacatgttttcaacaagtccctcaagacatgttgctgctgtcggtctgcagactacatgctggatagagagagtctaatggaataagtactgcaaactgggaaaacggtagtcatgatacct

SEQ ID NO: 853 - DNA, 203 bp, Homo sapiens
aagacatgttgctgctgtcggtctgcagactacatgctggatagagagagtctaatggaataagtactgcaaactgggaaaacggtagtcatgatacctacgtctttacttatgcctttttgtgcaaaatgtctttattttagcttcttcacttgaaaacaaagtgagggagaggaggttaccttgctaagtttcttctacag

SEQ ID NO: 854 - DNA, 202 bp, Homo sapiens
  misc_feature 1: n = A,T,C or G
  misc_feature 1: n = A,T,C or G
  misc_feature 1: n = A,T,C or G
nctttacttatgcctttttgtgcaaaatgtctttattttagcttcttcacttgaaaacaaagtgagggagaggaggttaccttgctaagtttcttctacaagatgagtgtttactcctagttggggcgtatatacacaatacctttaatatgaaaatatttatgttactacttataatgtatgcatacatattttctgctta

SEQ ID NO: 855 - DNA, 202 bp, Homo sapiens
ctatttaaagaaatcacagtaggatatgatttatttctcacaagccgtgaaatttcctttcaatataacaaggcatttgggggttaaaaattagatttttataccttgtacatttgtttaagttatttgtagattctggctattagccctttgtcaggtgggtagattgcaaaaattttctcccattctttaggttgcctgt

SEQ ID NO: 856 - DNA, 202 bp, Homo sapiens
tgttgccattgcttttagtgttttagtcatgaggtctttgcccatgcctatgtcctaaatggtattgcttaggttttcttctagggttgttatggttttcaggtcttacaaacaaacccataaaaaagtgggcaaaggatatgaacagatatttttcaagagaagacatttatgcagccaacagacacatgaaaaaatgctc

SEQ ID NO: 857 - DNA, 202 bp, Homo sapiens
tgcctatgtcctaaatggtattgcttaggttttcttctagggttgttatggttttcggtcttacaaacaaacccataaaaaagtgggcaaaggatatgaactagatatttttcaagagaagacatttatgcagccaacagacacatgaaaaaatgctcatcatcactggtcatcagagaaatgcaaatgaaaaccacaatgc

SEQ ID NO: 858 - DNA, 202 bp, Homo sapiens
cactctgggaggccgaggtggatggattgcttgaactcaggagtttgagaccagcctgtgcaacatggcaaaactctgtctctacaaataaatacaaaaaacttagctaggtgtggtggtgcacgcctgtggtcccagctactcaggaaactgaggctagaggatcacttgtgtctgggaggtcaaggctgcagtgagccaa

SEQ ID NO: 859 - DNA, 202 bp, Homo sapiens
aattagctaggtgtggtggtgcacgcctgtggtcccagctactcaggaaactgaggctagaggatcacttgtgtctgggaggtcaaggctgcagtgagccagagattgcacctctgcactccagcctggccagcagagtaagactctgtttcaaaaaaataaaaaatgaaaataataaaaaaaaagagacctgtaatgccag

SEQ ID NO: 860 - DNA, 202 bp, Homo sapiens
tgggaggtcaaggctgcagtgagccaagattgcacctctgcactccagcctggccagcagagtaagactctgtttcaaaaaaataaaaaatgaaaataatgaaaaaaaaaagagacctgtaatgccaggcactgtcattttcttgtagaatagggcattccatcagaagttatctaatagttggagcctcatactcctgatt

SEQ ID NO: 861 - DNA, 202 bp, Homo sapiens
cagagtaagactctgtttcaaaaaaataaaaaatgaaaataataaaaaaaaagagacctgtaatgccaggcactgtcattttcttgtagaatagggcattctcatcagaagttatctaatagttggagcctcatactcctgattattccataagccaatgactaatatttcacaatgataatgtctttttactggattccca

SEQ ID NO: 862 - DNA, 202 bp, Homo sapiens
ctaatatttcacaatgataatgtctttttactggattcccaaaaacatattaccacattttgataatttaaagattcacacttaaaaaatgttttcattcagttttctttttgatacggtttggctgtgtccccacccagatctcatcttgaattcccacatgttgtgggaaggacctggtgggaggtaattgaatcctggg

SEQ ID NO: 863 - DNA, 202 bp, Homo sapiens
tttcacaatgataatgtctttttactggattcccaaaaacatattaccacattttgataatttaaagattcacacttaaaaaatgttttcattcgttttcctttttgatacggtttggctgtgtccccacccagatctcatcttgaattcccacatgttgtgggaaggacctggtgggaggtaattgaatcctggggtcagg

SEQ ID NO: 864 - DNA, 202 bp, Homo sapiens
atacggtttggctgtgtccccacccagatctcatcttgaattcccacatgttgtgggaaggacctggtgggaggtaattgaatcctggggtcaggtctttctccatgctgttcttgtgatagtgaataagtctcgcgagatctgatggttttaaaaagaggaatttccctgcacaagctctcttctctcgtctgctgccacg

SEQ ID NO: 865 - DNA, 202 bp, Homo sapiens
acccagatctcatcttgaattcccacatgttgtgggaaggacctggtgggaggtaattgaatcctggggtcaggtcttttccatgctgttcttgtgatagctgaataagtctcgcgagatctgatggttttaaaaagaggaatttccctgcacaagctctcttctctcgtctgctgccacgtgagacatgcctttcaccttg

SEQ ID NO: 866 - DNA, 202 bp, Homo sapiens
aaggacctggtgggaggtaattgaatcctggggtcaggtcttttccatgctgttcttgtgatagtgaataagtctcgcgagatctgatggttttaaaaagagggaatttccctgcacaagctctcttctctcgtctgctgccacgtgagacatgcctttcaccttgagccatgattgtgaggcttccccaaccacgtggaat

SEQ ID NO: 867 - DNA, 202 bp, Homo sapiens
caagctctcttctctcgtctgctgccacgtgagacatgcctttcaccttgagccatgattgtgaggcttccccaaccacgtggaattgtaagtccattaaagcctctttcttttgcaaattgcccagtcttgggtattctttatcagcagtgtgaaaacagactaataacactttttgtgatagattaaattttccatgtac

SEQ ID NO: 868 - DNA, 202 bp, Homo sapiens
acagaagcctagatctccccatagaagaaggtgctttgggattgctggtttctgtagtcttcttttaccagcaggatggagacagggtccctgatgagaactggggtgattgctaagggtgttggtatggtgagaattcagggtgaggctgctgaccagggtactgagaggggaaattctgagaagtgcagctgcctgccca

SEQ ID NO: 869 - DNA, 202 bp, Homo sapiens
gctgaccagggtactgagaggggaaattctgagaagtgcagctgcctgcccattccggtgaagagtgaagatggacagagctttgcactgaccctgttgcctcttcccgctggggtggccagtgtggaatccaatgagctactaacaaggacagttacctttgcagataaaacaacagtaatttagaggagaattgcaattt

SEQ ID NO: 870 - DNA, 202 bp, Homo sapiens
tttattagctcatgcctttctacggtaatgcatcatctttcatgggattgtagagaagctcagtgcaataatttctctggttcaattcctttgctacatactaattaatctgcattgataactatggtattttggggatgtatcatttgggaattgcattctcaaatacctttattgtgatgctaaagagtcccaaatcata

SEQ ID NO: 871 - DNA, 202 bp, Homo sapiens
tgcctttctacggtaatgcatcatctttcatgggattgtagagaagctcagtgcaataatttctctggttcaattcctttgctacatataattaatctgcagttgataactatggtattttggggatgtatcatttgggaattgcattctcaaatacctttattgtgatgctaaagagtcccaaatcatagtatattaaacc

SEQ ID NO: 872 - DNA, 202 bp, Homo sapiens
tcccaaatcatagtatattaaaccagatatatatatttcctttcatataacaagagagttgaaaggaggtagtgcagagatgaaatgaaagtccatagcccttaccacggtcttagcgggttctttccttctgttttaccatcattagcatatggttccctgatggttgtttcagaatagaacgatggctgctacaacaacc

SEQ ID NO: 873 - DNA, 202 bp, Homo sapiens
attaaaccagatatatatatttcctttcatataacaagagagttgaaaggaggtagtgcagagatgaaatgaaagtccatagccctaccacggtcttagcagggttctttccttctgttttaccatcattagcatatggttccctgatggttgtttcagaatagaacgatggctgctacaacaaccagccatagcatccatt

SEQ ID NO: 874 - DNA, 202 bp, Homo sapiens
atcgggtaggcaagtggtagactctggctaccactgtgcggcattttagtgaatgaaagatgacgatgtggcatgtatattcttagaaaggccatgtaagactgatgttatctatgggtcaaatgaatttcccttcaggtgaccaaggtatttacacacattccttgcaggcattaaaaagaaaaaaggaaggagggcaggg

SEQ ID NO: 875 - DNA, 202 bp, Homo sapiens
gatgtggcatgtatattcttagaaaggccatgtaagatgatgttatctatgggtcaaatgaatttcccttcaggtgaccaaggtatttacacacattcctgtgcaggcattaaaaagaaaaaaggaaggagggcagggagaaagccactgccacagttgtggccgagtgaaaggtgaacacctaggtgtggtagttccagta

SEQ ID NO: 876 - DNA, 202 bp, Homo sapiens
aaaaagaaaaaaggaaggagggcagggagaaagccactgccacagttgtggccgagtgaaaggtgaacacctaggtgtggtagttccagtatggcagatgagaattgttcactctttcagtgcccccttgtgcccaatatactgtaacaggataatacagggaaacagtcaagaaagaaactttttgaaaactccattcatt

SEQ ID NO: 877 - DNA, 202 bp, Homo sapiens
aggaaggagggcagggagaaagccactgccacagttgtggccgagtgaaaggtgaacacctaggtgtggtagttccagtatggcagatggaattgttcacatctttcagtgcccccttgtgcccaatatactgtaacaggataatacagggaaacagtcaagaaagaaactttttgaaaactccattcattaaagctacagt

SEQ ID NO: 878 - DNA, 202 bp, Homo sapiens
cccttgtgcccaatatactgtaacaggataatacagggaaacagtcaagaaagaaactttttgaaaactccattcattaaagctacagtatagcaatgtggtggttttagctactagaaatgcaaaaaaataaaaaatacctatacagtatataacttcatgatgctgggacaagacgtcacaatgttaaggcatatctttc

SEQ ID NO: 879 - DNA, 202 bp, Homo sapiens
tgaaaactccattcattaaagctacagtatagcaatgtgtggttttagctactagaaatgcaaaaaaataaaaaatacctatacagtatataacttcatgagtgctgggacaagacgtcacaatgttaaggcatatctttcttgtgctaagtagttctcttgtcaaaaaatatgttttccatttaaatttggagttgttggg

SEQ ID NO: 880 - DNA, 202 bp, Homo sapiens
tattaactccgaactgttgccaccatttacagagactcataggagaagccgcttgctctacagtgtgaggattgcaagctgtgctaggattcaagctcagacgtccctgagcctttgtctcctgttctttcctgtgtgcttatgtggtcagtccactttccttctccgacatgaaggcttggcccattccaagtctcctttg

SEQ ID NO: 881 - DNA, 201 bp, Homo sapiens
gtcaatttaaacagtggaagagtaattcatcacaggggaagaggaatgagaacctttggaatttattttcaataaaagatcctgctagtagaagaaaactctcctgaaaccctggttttactgatgcccattaacatgtatacttgaagcaagagtatgaagctcttaaaatctgccctttcaaaaactttaaaaagttat

SEQ ID NO: 882 - DNA, 202 bp, Homo sapiens
aaagatcctgctagtagaagaaaactcctgaaaccctggttttactgatgcccattaacatgtatacttgaagcaagagtatgaagctcttaaaatctgccgctttcaaaaactttaaaaagttatgtagggagttataagtacataagatgaaaatgaacaccttctcttagtaaatgtattttaggagcatagagaaatt

SEQ ID NO: 883 - DNA, 202 bp, Homo sapiens
ttgaagcaagagtatgaagctcttaaaatctgccctttcaaaaactttaaaaagttatgtagggagttataagtacataagatgaaaatgaacaccttctctttagtaaatgtattttaggagcatagagaaattgtaaagagaaaggtgagagtaatagaacttcacctggtaattttctggggtaaaagaaaaggggata

SEQ ID NO: 884 - DNA, 202 bp, Homo sapiens
atagaatcttaacacccacttcctctgttcagttatgtctttgctccttcaagtatggtctgcaggctggtcatcacctgggagcttgtttgaaatgcaaactctcaggccctcactctgacttgtgcaaccagaatcaacattttcaaaatatctttgggtaaactgtatgcacattaaagtttaagaagctgtcagtcat

SEQ ID NO: 885 - DNA, 202 bp, Homo sapiens
ttcaaaatatctttgggtaaactgtatgcacattaaagtttaagaagctgtcagtcatctatataacttctattcacatggaaattctgattatgaacctagctgcttaaatctaagatattcttcacatgacttatacacctattctggaaaatatctgaaagtacccgggaataaaatgcatgagactatctgtgtatat

SEQ ID NO: 886 - DNA, 202 bp, Homo sapiens
aaagtttaagaagctgtcagtcatctatataacttctattcacatggaaattctgattatgaacctgctgcttaaatctaagatattcttcacatgacttattacacctattctggaaaatatctgaaagtacccgggaataaaatgcatgagactatctgtgtatatatagctttctatgtttctggttgacttttctttt

SEQ ID NO: 887 - DNA, 203 bp, Homo sapiens
atatatagctttctatgtttctggttgacttttcttttttttactattttatggcaataaatattttattgaagcagcttgggttttttttttttttttttttgattggttaactgacttattcatcccacaaatctttattaaacacttcatgtgccagtaaataaaaaaagatgatgaagccatgccgatctcctcaagaa

SEQ ID NO: 888 - DNA, 202 bp, Homo sapiens
aagtgctgggattacaggtgtgagcaactgcacccagcctatgattcttagaggtattttaaatctcctgaaattgtacataaaattccatttttatgtgactgttttctgggaggagaaaccataatttgaatctggtaccaatgaaattcactccccaaaaggagatctggcaggtgataatgattttgaaggtttacat

SEQ ID NO: 889 - DNA, 202 bp, Homo sapiens
tatagctctctgtattctttttcttgaataatcttggttgtctttacaaatactagcaattttagtgctttctttgtaatattttgtgctttaaaatatgatgtctttggtcaggcgtggtgcctcatgctgtaatcctagcactttgcgaggccaaggtgggaggatcgcctgaggccaggagttcaagattaggctgagc

SEQ ID NO: 890 - DNA, 202 bp, Homo sapiens
atctcagtcacatatacagaactagatttgacaaccagggaacattctaggttagtaaaagttggcatgcaacttctttgaaactgagttacttgttagactgatgaaaacttagaaaaactctttttcttttcttttgggtagagctttttgtagattgttttacaatgtacatgatggtttcttattttttaaagctggg

SEQ ID NO: 891 - DNA, 202 bp, Homo sapiens
aggatgctttttagtgatcagatctcattgtaacgaacagagtgagtaaccctcgattactgtgggaggggcaccaagctacttatgagggatcttccccagataagccaaacacctcccagcaggccccacctctaactgtggggagtcacgttttcacgttaggtttagtgggaacaaatattgaaactgtatcagttgt

SEQ ID NO: 892 - DNA, 202 bp, Homo sapiens
aacgaacagagtgagtaaccctcgattactgtgggaggggcaccaagctacttatgagggatcttccccgataagccaaacacctcccagcaggccccaccttctaactgtggggagtcacgttttcacgttaggtttagtgggaacaaatattgaaactgtatcagttgtaataagtgtttttttggaaaaaaaaaaaaaa

SEQ ID NO: 893 - DNA, 202 bp, Homo sapiens
tgtgcaaatagggagaccagttgaatgtgcaagacactatacggaaattgaatattgatcaagactggtggtaaggaataggaaaagctgtcataaagctagaataaccaaccattgcattaaattccaacttcaaggcacaggcccttcttgtcatgccaggtagtcagtaagtttcagaaggtttaaaaatactcctcct

SEQ ID NO: 894 - DNA, 202 bp, Homo sapiens
aattccaacttcaaggcacaggcccttcttgtcatgccaggtagtcagtaagtttcagaaggtttaaaaatactcctcctgggtgaataatagtaaaggcactctaaattttcttcaatgagtgctctgcaaggtacagctgtcaaaagataaccaaggggcaagttgtaccttaggcatggatgccatggtcactaagatg

SEQ ID NO: 895 - DNA, 202 bp, Homo sapiens
ttggtttttcatcatcatgctgcttatttcatagtcacaagatggctgctgcagctccaggcattacttcttcatagtagagtcacattcagaaaggcaggtgggaggggcaagacacactctcctcattagacttgctattttttttcccttggaagaacagcccctaacagacaacctctcacctctcactggtcagtag

SEQ ID NO: 896 - DNA, 202 bp, Homo sapiens
aacctctcacctctcactggtcagtagaggacttcaggagatttctagatccttttgctttgttgacaaagagcaaaggattaccttgcctgttatatagacgcaatcatgaatcatctcctgtggctaagaggaggccacattccccaagatcaaggtctctttacctgctcttggacacatcaggattctattagcaggt

SEQ ID NO: 897 - DNA, 202 bp, Homo sapiens
tccccaagatcaaggtctctttacctgctcttggacacatcaggattctattagcaggtatgaagtacacttgtgtttgtggtgaagggtagagggaatgcgggcagaaatgacaattgggtatggtagtggctgagtgaatacttttgaggggatagtttgggctcgttcttctagacttcatatttaagctgtgacctaa

SEQ ID NO: 898 - DNA, 202 bp, Homo sapiens
ctaaatctttcaaaggaaaatgaacacaatctggtttgctggaggaacagaagtatacccacagtgttggcagtgagggaacatgaggttgacagctgggacaggaaccagattaagagattggatcttattttaagtgcaatggggagccattgtaggttttaagtacaagaataatttatgttttaaaaaagatagttct

SEQ ID NO: 899 - DNA, 202 bp, Homo sapiens
ataaaggctatgacttccatcttggtctctctctttcttggatctcttgctctgaaagaaacaagctcctaagttttgagctaccttaaggagaggcctggtgtggcaaggatcaacagccagcaagaaatggaggcctgccaacagctacacaagtggccccagaagtagacccttttttcaatagagatgacagttggca

SEQ ID NO: 900 - DNA, 202 bp, Homo sapiens
aaaatttcttaaacttctttatgatgtgacatttatacactgagagtgtgtgttaaggttgctgtggggactttaactcttgaacttcctgatacttggtagtattttcattttaaaccttcctattctatcacaataaatttacaagttaaacatgtatagctatgcaccgtggctttgaatgtgtctgaatttccaagga

SEQ ID NO: 901 - DNA, 202 bp, Homo sapiens
gtattttcattttaaaccttcctattctatcacaataaatttacaagttaaacatgtatagctatgcaccgtggctttgaatgtgtctgaatttccaaggacaacatgcactatggtattggctatttatgagtgtggagaaatctaaggaaacgtattttcatttcttattcctcagcaaattacagctcgacatgggcct

SEQ ID NO: 902 - DNA, 202 bp, Homo sapiens
atattgaacttcttgaagggaaagtataagtagcatcctcttcttttgctttctggacacatattttcccatctccagaaagactgagtccccagggaatagaagactattgatgtggccttaagtttgcaatttgtgcttcccataaaaattcctcccccaactatgtatgtatgtcaatcatgatatgtcaccagcaggt

SEQ ID NO: 903 - DNA, 202 bp, Homo sapiens
ttattaatcagagggaggtccttttaatagcctgtaggtcttttcctctacatcagggacagggaggtttgcccttgtttgttaaagagttctcattttcctgacttgcctgcccctatgggcctcttaaagcaactctgaaaacaagtggcaaagaataattttttactagaagacttagcaagagccaactaatttgacc

SEQ ID NO: 904 - DNA, 202 bp, Homo sapiens
agaagacttagcaagagccaactaatttgacccattaagtgggctatcagaaagctaggaggagatgacaggggttgtttgccaatatgaaagaaagagaagatatcactgttttttttttctttttttttctttgtttgtttgtttgtttgtttttttgttaagctggcagctgctgtagtcggattcactggcattgact

SEQ ID NO: 905 - DNA, 202 bp, Homo sapiens
gatactatatggcagagcaggcatgcttgggcttgtcacatgtacaatgagggactagagctgtctttcaggtccaaggagcaaggttatttgaggaaagcgtcagctgggagtgagtgttcttgaggaaacatctgctccatagggatgtagacccttagaagaacatcctttgcaggttgtgcatgagacaagccataaa

SEQ ID NO: 906 - DNA, 202 bp, Homo sapiens
gtctttcaggtccaaggagcaaggttatttgaggaaagctcagctgggagtgagtgttcttgaggaaacatctgctccatagggatgtagacccttagaaagaacatcctttgcaggttgtgcatgagacaagccataaattagtaggatgagctccccctgtagtctactctgagtacagaactaccttacagacaattca

SEQ ID NO: 907 - DNA, 202 bp, Homo sapiens
gaaagcgcattataagaaatcaggctatgactttggttttgccgatagctgacatttcaaattcgggcaagtcacaaaaatttcatttccacttacttatagttaagtagggtaatgatcccgccttggagaccctatagaatatttgatgataataataataataactaaatgattatcgcttattcatgtgctgatttcc

SEQ ID NO: 908 - DNA, 202 bp, Homo sapiens
ctaaatgattatcgcttattcatgtgctgatttccagcctgatagtcaggctcttcacccatctatttcagagaaaagccaatatcctaccaccatctccaggggccctatatcatctgacccctttacttctgaccttgtttcctcctgttctccttttattcattcaaactaagaaactagctttcttgttatgtcttga

SEQ ID NO: 909 - DNA, 202 bp, Homo sapiens
ctgaccttgtttcctcctgttctccttttattcattcaaactaagaaactagctttcttgttatgtcttgaatacactagggtctttgcattgcctatgtagctcttcctggactgctcgttctccaaatatgcagacgatagaatttcttaccatctttccatctttgttcaaatgctgccttcttaattaagtctacctg

SEQ ID NO: 910 - DNA, 202 bp, Homo sapiens
ttaccatctttccatctttgttcaaatgctgccttcttaattaagtctacctggcccacacattttacagttcgaacctgtccccaaccagactcctaaccgtcttcattttccacattcaaaagcacttacctgttaattatgattattgtttactgtctgtctcctccaattagaatataaattctttaagtactggaat

SEQ ID NO: 911 - DNA, 202 bp, Homo sapiens
agttcgaacctgtccccaaccagactcctaacctcttcattttccacattcaaaagcacttacctgttaattatgattattgtttactgtctgtctcctcctaattagaatataaattctttaagtactggaatttttgtttcgttggtttgttttcaatggagtatccaaataccttgaagtgcttaacatacaaagggat

SEQ ID NO: 912 - DNA, 202 bp, Homo sapiens
tggaatttttgtttcgttggtttgttttcaatggagtatccaaataccttgaagtgcttaacatacaaagggatttgattaatatttggcaaatgaatgactaacacagccgtcctgttaggtacacacacaaaaacacacatgtatacatatattcacacactcccactctcacatacacttgtatacagaatttagattt

SEQ ID NO: 913 - DNA, 202 bp, Homo sapiens
cttaacatacaaagggatttgattaatatttggcaaatgaatgacaacacagccgtcctgttaggtacacacacaaaaacacacatgtatacatatattcatcacactcccactctcacatacacttgtatacagaatttagatttattttataacttgatgaggttgattaataacccattctgtgctcctattacaaaag

SEQ ID NO: 914 - DNA, 202 bp, Homo sapiens
gtttgaattgtcacaaattacaaatcacaaggaatcttaactgatgagtttcttccttgaaaatgtgttactccacaatgtaaacatcaggtgctttgatctgcttttggcttactcatattcatgcttctttatccaggatccttccaaataaagcatccagaaaatgtttgagtcacaaaaatatgggctttgtattgtt

SEQ ID NO: 915 - DNA, 202 bp, Homo sapiens
cttgaaaatgtgttactccacaatgtaaacatcaggtgctttgattgcttttggcttactcatattcatgcttctttatccaggatccttccaaataaagctatccagaaaatgtttgagtcacaaaaatatgggctttgtattgtttacaaaggaatattggtggtagagaggagggatggatttcagttctacttcactc

SEQ ID NO: 916 - DNA, 202 bp, Homo sapiens
atttcagttctacttcactcacctcacatttggtttagcgtgtcctatttttgggctatcattacatgaaggaagcctaccaacttttgccaggcactttagcatcttcccttgttttgagtgagaagctctagattcttccccatgtcttctaccacagctttaagatggctatttggctttgactgaacactccagcgga

SEQ ID NO: 917 - DNA, 202 bp, Homo sapiens
cagttctacttcactcacctcacatttggtttagcgtgtcctatttttgggctatcattacatgaaggaagcctaccaacttttgccaggcactttgcatctttcccttgttttgagtgagaagctctagattcttccccatgtcttctaccacagctttaagatggctatttggctttgactgaacactccagcggagtct

SEQ ID NO: 918 - DNA, 202 bp, Homo sapiens
aacaaactgtccacatattagacaaatctagccaaaaatcaagtccaactctttggtcatagatgaacacttgtgaaggagggtaagtgaaatgcacacactctacccatgaaaatggcattactagaagtaatgacaatttgaagccataatcgtgatgctttagcactttctacttgatcaaaaataaggagatgtgggc

SEQ ID NO: 919 - DNA, 202 bp, Homo sapiens
ggcattactagaagtaatgacaatttgaagccataatcgtgatgctttagcactttctacttgatcaaaaataaggagatgtgggccaggtatggtggctctacacctataatcccagcactttgggaggccgaggctggtggatgacttgaggccaggagttcgagaccagtctggccaacctggtgaaatcatctctact

SEQ ID NO: 920 - DNA, 202 bp, Homo sapiens
aacagagagtgactcagggaaaaaaaacgaacaaacaaaaaaccaaggagatgtggagagagtttgccatctttggcaaaaagtgaagatcactaatttgagtgatcttaatttgatctctaaatgttgttttgctttgaaaaatagctgaacacatagcaatgttcagtttaaaaatcataattaatgaaattatgtatct

SEQ ID NO: 921 - DNA, 202 bp, Homo sapiens
gttttgctttgaaaaatagctgaacacatagcaatgttcagtttaaaaatcataattaatgaaattatgtatcttttctcagacccagatcacagaatcacttgggaggagattaaatattgggatagagcagagtaaaaagaaccatgaaggaaaatcatctttaacttgaaactaaatttggtaacatctaatatcaaac

SEQ ID NO: 922 - DNA, 202 bp, Homo sapiens
gggaatgggattgtaaattaatggtttacatagctgtcacctgaattcctagaattggaacattttgacttccttgatgagctcagacttttgcttcatgctcacttatgaacatatttccaaaatttggaaaataaatgagtttagtttcaaagtttataaattttttttgtgaatttagagagagaattaaagtggtgga

SEQ ID NO: 923 - DNA, 202 bp, Homo sapiens
ttatgaacatatttccaaaatttggaaaataaatgagtttagtttcaaagtttataaattttttttgtgaatttagagagagaattaaagtggtggaatactggttacaaagtaatgttattagctataattaatcagcacggacaagtattgtgtcctaggataaaattttaatttgcaaggtctcaattaagagtgaata

SEQ ID NO: 924 - DNA, 202 bp, Homo sapiens
gagtgaatagaagctgtcagtgaatttatatataattttgtaaattcagtagatagcaacctatgacattatgcagatgactgttttgcatatttattaactttttttcatattttccttattgggaatcaagaaatttgtgcatattaaagttacagagtgtgccacattatgacaaagactgtgacctctttaagaaatg

SEQ ID NO: 925 - DNA, 202 bp, Homo sapiens
tgacattatgcagatgactgttttgcatatttattaacttttttcatattttccttattgggaatcaagaaatttgtgcatattaaagttacagagtgtgctcacattatgacaaagactgtgacctctttaagaaatgtttcccggtcgggtgcatggctcacacctgtaatcctagcactttgggaggctgaggtgggcg

SEQ ID NO: 926 - DNA, 202 bp, Homo sapiens
tattttccttattgggaatcaagaaatttgtgcatattaaagttacagagtgtgccacattatgacaaagactgtgacctctttaagaaatgtttcccggctcgggtgcatggctcacacctgtaatcctagcactttgggaggctgaggtgggcggatcacctgaggtcaggtgttcgagaccagcctggccaacatggca

SEQ ID NO: 927 - DNA, 202 bp, Homo sapiens
tgcatattaaagttacagagtgtgccacattatgacaaagactgtgacctctttaagaaatgtttcccggtcgggtgcatggctcacacctgtaatcctaagcactttgggaggctgaggtgggcggatcacctgaggtcaggtgttcgagaccagcctggccaacatggcaaaaccctgtctctactaaaaatacaaaaat

SEQ ID NO: 928 - DNA, 202 bp, Homo sapiens
aggctgaggtgggcggatcacctgaggtcaggtgttcgagaccagcctggccaacatggcaaaaccctgtctctactaaaaatacaaaaattagccaggcagtggtggtgcatgcctgtaataccagttactcaggaggctgaggcaggagaatcactgaaacccaggaggcggaggttgtagtgagctgagattgtaccac

SEQ ID NO: 929 - DNA, 202 bp, Homo sapiens
catggcaaaaccctgtctctactaaaaatacaaaaattagccaggcgtggtggtgcatgcctgtaataccagttactcaggaggctgaggcaggagaatcagctgaaacccaggaggcggaggttgtagtgagctgagattgtaccactgcactccagcctcggcgacagagtgagcctccatctgaaaaaaaaaagaaaga

SEQ ID NO: 930 - DNA, 202 bp, Homo sapiens
tgtaataccagttactcaggaggctgaggcaggagaatcactgaaacccaggaggcggaggttgtagtgagctgagattgtaccactgcactccagcctcaggcgacagagtgagcctccatctgaaaaaaaaaagaaagaaagaaagaaagaaagaaagaaagaaagaaagaaagaaagaaagaaagaaagagagaaagaa

SEQ ID NO: 931 - DNA, 202 bp, Homo sapiens
gcggaggttgtagtgagctgagattgtaccactgcactccagcctcggcgacagagtgagcctccatctgaaaaaaaaaagaaagaaagaaagaaagaaaagtgtttcccagtttttcatggcctctgatgatttgaacaaaggcacttgttaaatagaagatgagatacacatgacacctttgctcagaagtcctctcatc

SEQ ID NO: 932 - DNA, 202 bp, Homo sapiens
atctgaaaaaaaaaagaaagaaagaaagaaagaaagaaagaaagaaagaaagaaagaaagaaagaaagaaagagagaaagaaagaaagaaagaaagaaaaatgtttcccagtttttcatggcctctgatgatttgaacaaaggcacttgttaaatagaagatgagatacacatgacacctttgctcagaagtcctctcatct

SEQ ID NO: 933 - DNA, 202 bp, Homo sapiens
gaaagaaagagagaaagaaagaaagaaagaaagaaaatgtttcccagtttttcatggcctctgatgatttgaacaaaggcacttgttaaatagaagatgaagatacacatgacacctttgctcagaagtcctctcatctctctgtgccagacaattttaaaataagatgtgatttcttccccctattctcaaataatatatt

SEQ ID NO: 934 - DNA, 202 bp, Homo sapiens
tggcctctgatgatttgaacaaaggcacttgttaaatagaagatgagatacacatgacacctttgctcagaagtcctctcatctctctgtgccagacaatctttaaaataagatgtgatttcttccccctattctcaaataatatatttgctattatggaaaacatgttaatttgtgaaaagtattaaaaaaaatcagcctg

SEQ ID NO: 935 - DNA, 202 bp, Homo sapiens
aagatgagatacacatgacacctttgctcagaagtcctctcatctctctgtgccagacaattttaaaataagatgtgatttcttccccctattctcaaatagatatatttgctattatggaaaacatgttaatttgtgaaaagtattaaaaaaaatcagcctgatcctattgcttaaaactagcaatgatagttaattaatt

SEQ ID NO: 936 - DNA, 202 bp, Homo sapiens
ttgttgcccaggctggagtgcagaggtgtgatcacagctcacagcagcctcaacctcctgggctcaggtgatcctcccacctcagcctcctgagtaactgaggactccaggcacatgccatcatgcctggctaattttttgtagtttttgcagagacagtgtttcaccatgttgctcaggctggtcttgaactcctgaactc

SEQ ID NO: 937 - DNA, 202 bp, Homo sapiens
tgcccaggctggagtgcagaggtgtgatcacagctcacagcagcctcaacctcctgggctcaggtgatcctcccacctcagcctcctgagtaactgagacatccaggcacatgccatcatgcctggctaattttttgtagtttttgcagagacagtgtttcaccatgttgctcaggctggtcttgaactcctgaactcaagt

SEQ ID NO: 938 - DNA, 202 bp, Homo sapiens
tcccaaagtgctaggattacagggttgagttactgtggccagccaactgttaatattttaatgtttctttctcatcttatgtctaccactgcatattataaggtctatattactttgtgtttgactttttacttcatattattgttttgtatattatgagctttgcatattatgagctctttctaaatatattttgtcatat

SEQ ID NO: 939 - DNA, 202 bp, Homo sapiens
actattgtttagatattgggaatagatttttgaaagctggacttcagactgggagacttactggaaaataaaaccaccaagaaaaaaactttgaaatttactgaaactttctaaagaaaagttatatattttagattttcctaagttaatgttaggcctgaaggcaagttgaaaatgataaatttttaaggaccaatttttt

SEQ ID NO: 940 - DNA, 235 bp, Homo sapiens
caaagccatagcaattgagacagcatgtactagcttaaggatatctctctctctctctctctctctctctctctctatcatctatctgtctgtctgtcttatctatctaaatggaatagaatcgagggtttacaaataaacccttattatttgtgctcaactgacttttaaacaaagatagcaaggcaattcaatgaaaaaagaacagtttttcaacgagcagtcatgggagaat

SEQ ID NO: 941 - DNA, 22 bp, Homo sapiens
caaagccatagcaattgagaca

SEQ ID NO: 942 - DNA, 20 bp, Homo sapiens
attctcccatgactgctcgt

SEQ ID NO: 943 - DNA, 209 bp, Homo sapiens
ttggtttggagcttcagaaattaaacattcttgaataaagaatgggtctttcactaaataaaattcctgcttattgtgaaaaatatcattgccagttttttgtgttttttttttgtttgtttgtttgtttgtttttttttacacagggcagattcttttctccttcaattttttatccctcttttacttaagccaccctttaaagaccc

SEQ ID NO: 944 - DNA, 21 bp, Homo sapiens
ttggtttggagcttcagaaat

SEQ ID NO: 945 - DNA, 21 bp, Homo sapiens
gggtctttaaagggtggctta

SEQ ID NO: 946 - DNA, 255 bp, Homo sapiens
gacccacctgcaagaagtgtgcagaagaatgaatgtggcctgtgattgggcagccacttccttgttacaactgtgttctatggaaaaggagaaaagtttgtggtggatagatagccattgctgcaggaggaattggaaatagcagaattccatctcaagtatctgtagtattcactgccagatagatagatagatagatagatagatagatagatagatagatagatagataaactttccaaatagatctttgcc

SEQ ID NO: 947 - DNA, 20 bp, Homo sapiens
gacccacctgcaagaagtgt

SEQ ID NO: 948 - DNA, 20 bp, Homo sapiens
ctgagccttgaaggatggat

SEQ ID NO: 949 - DNA, 100 bp, Homo sapiens
ccatctcaagtatctgtagtattcactgccagatagatagatagatagatagatagatagatagatagatagatagatagataaactttccaaatagatc

SEQ ID NO: 950 - DNA, 20 bp, Homo sapiens
ccatctctagtatctgtagt

SEQ ID NO: 951 - DNA, 18 bp, Homo sapiens
gatctatttggaaagttt

SEQ ID NO: 952 - DNA, 214 bp, Homo sapiens
cagccactatggccttacaaagaaatgtgtgtgtgtgtgttcgtgtgtgtgtgtgtgtgtgtgtgtgtatatgtgtatatatatatatatatatatacttgcaattaatcaaattgtttaatgtctgcctgcttagtaacaaagagattacatagtttgttgttattattgcttgtaaatgaccttgcagagtttctttacaatccattgggca

SEQ ID NO: 953 - DNA, 21 bp, Homo sapiens
cagccactatggccttacaaa

SEQ ID NO: 954 - DNA, 21 bp, Homo sapiens
tgcccaatggattgtaaagaa

SEQ ID NO: 955 - DNA, 194 bp, Homo sapiens
ttcctctatccctcccttgccccttctttctttcctttacttccttccttccttccttccttccttccttccttccttctttccttccttctttctttcatttaatcatcctgataggacactgaactaaaagcttgaaccctgggtttctgttctgatctaccactcatagctgtgtgtccttgaacaatccc

SEQ ID NO: 956 - DNA, 20 bp, Homo sapiens
ttcctctatccctcccttgc

SEQ ID NO: 957 - DNA, 21 bp, Homo sapiens
gggattgttcaaggacacaca

SEQ ID NO: 958 - DNA, 203 bp, Homo sapiens
ctcttccttcctgcgtccttccctcatttctttcctttcttccttcctcccacttccctccctccctctctctctcccttccttccttccttccttccttccttctttcctttatttctttcttccttcctctttttttcctaccttatctcgtttccagatatctgcctaatttctcctagttctggtgctacatcctcctc

SEQ ID NO: 959 - DNA, 20 bp, Homo sapiens
ctcttccttcctgcgtcctt

SEQ ID NO: 960 - DNA, 21 bp, Homo sapiens
gaggaggatgtagcaccagaa

SEQ ID NO: 961 - DNA, 142 bp, Homo sapiens
tttgttgagggaaggacaggtggattgataaatggatgggtggacggatgaatggttgggtggatgaatggttggatggatggatggatggatggatggatggatggatggaagacaaatatactgatggatggtgtgctga

SEQ ID NO: 962 - DNA, 20 bp, Homo sapiens
tttgttgagggaaggacagg

SEQ ID NO: 963 - DNA, 20 bp, Homo sapiens
tcagcacaccatccatcagt

SEQ ID NO: 964 - DNA, 255 bp, Homo sapiens
agctgggtcccaacactctacttcctctttttaaagaccctaccagactaagtaaccctaactagatccattacatactgtttatttccttccttccttccttccttccttccttccttccttccttccttccttctttcctcctccctccctccgtccctccatctcttcttcccttcctgcctctcttcctcccttcctcccttcattttttcctctctctctctctctgtctgtctctctttctttctaaat

SEQ ID NO: 965 - DNA, 20 bp, Homo sapiens
agctgggtcccaacactcta

SEQ ID NO: 966 - DNA, 20 bp, Homo sapiens
gaccaaatctgggcttgaat

SEQ ID NO: 967 - DNA, 255 bp, Homo sapiens
tctgtcagcctgtagacgtgttttaatccatgaattccagtgtgatttgctatgtagaattattgtgaccatagatagttgatacaatatatatgtatgtgtgtacatatatagtatatatatatatatatatatgtttatgtgtatataggaaagcctgaaaaactttaattataatatttcatttccttctataagtaattgctacatgatattatgaatatttttaaaaatccaaaaatatattttcccaag

SEQ ID NO: 968 - DNA, 22 bp, Homo sapiens
tctgtcagcctgtagacgtgtt

SEQ ID NO: 969 - DNA, 20 bp, Homo sapiens
gtgcaagtcatcccagcttt

SEQ ID NO: 970 - DNA, 255 bp, Homo sapiens
tactctggccagcaggaacccaggagcccagaagccccaggcctgacccattctggcaattattataatattattcttaaaagccaagtccgtctctctctctctctctctctctctctctctctctctctctctctctctctcaagtccctctctctctctctttctctctctctcccccatcactctatctttctcacacacactcacatgctcacatgatcacacacacacagagaataacaagctttgcaa

SEQ ID NO: 971 - DNA, 19 bp, Homo sapiens
tactctggccagcaggaac

SEQ ID NO: 972 - DNA, 20 bp, Homo sapiens
tcatcctcagcctgaccttt

SEQ ID NO: 973 - DNA, 255 bp, Homo sapiens
gccgcctctgtttgttgtatagtattagtgaatcgcaatgtggcacacaccgatcatcctaaaaatatgtccagaaaagttgcatagattgtgtgtggccctgtgtgtgtgtgtgtgtgtgtgtgtgtgtgtgcatatgtatgtgtatgtttatacatctagcttcttcccccattctcccaccaacactgtttgggtagccagccccccatcctctctaaagcccccatgccctgcagtaaacactgaggccag

SEQ ID NO: 974 - DNA, 20 bp, Homo sapiens
gccgcctctgtttgttgtat

SEQ ID NO: 975 - DNA, 20 bp, Homo sapiens
ccctggcctcagtgtttact

SEQ ID NO: 976 - DNA, 177 bp, Homo sapiens
gttgctccagatgcagtgagaataaaaaagaaagaaagaaagaaagagaaagaaagaaagagagagagagaggaaaagaaagaaagaaactaactcaagaaaattgagtgtgcttctaagaaaagagttgagatggccatatagaagacccagagaagacacaaaggcaagaggtca

SEQ ID NO: 977 - DNA, 20 bp, Homo sapiens
gttgctccagatgcagtgag

SEQ ID NO: 978 - DNA, 21 bp, Homo sapiens
tgacctcttgcctttgtgtct

SEQ ID NO: 979 - DNA, 156 bp, Homo sapiens
ggtgagagggtgtcagcaatcaaaaccttgacttcagagagaggcagaaaaggacagagagagagagagagagagagagaatgagaacaacaaaaagaaactgcctaagggcacattgcttttacacaaggagttggtggaagagccaggagacag

SEQ ID NO: 980 - DNA, 20 bp, Homo sapiens
ggtgagagggtgtcagcaat

SEQ ID NO: 981 - DNA, 20 bp, Homo sapiens
ctgtctcctggctcttccac

SEQ ID NO: 982 - DNA, 255 bp, Homo sapiens
gtcgccctcatgaaacaatcatctcaatgccgttggagttttcaagcagaatctagtccaccatacaacaaacatgcggaaaagtgcatgcactggacaaaaagaccaacagatatatctatacatctatctatctatctatctatctatctatctatctatctatctatctatctatctatctatcatcagacacacaagggaagaatgccatgtgatgacagaggcagagactgaggtgctgcagctgcaagc

SEQ ID NO: 983 - DNA, 20 bp, Homo sapiens
gtcgccctcatgaaacaatc

SEQ ID NO: 984 - DNA, 20 bp, Homo sapiens
cccttcctagcttcttgtgc

SEQ ID NO: 985 - DNA, 177 bp, Homo sapiens
ttcttggcttgcagctgcagcacctcagtctctgcctctgtcatcacatggcattcttcccttgtgtgtctgatgatagatagatagatagatagatagatagatagatagatagatagatagatagatgtatagatatatctgttggtctttttgtccagtgcatgcacttttccg

SEQ ID NO: 986 - DNA, 19 bp, Homo sapiens
ttcttggcttgcagctgca

SEQ ID NO: 987 - DNA, 19 bp, Homo sapiens
cggaaaagtgcatgcactg

SEQ ID NO: 988 - DNA, 255 bp, Homo sapiens
aggtgacgttggagagagagagagagaaagtgtgtgtgtgtgtgtgtgtgtgtgtgtgtgattctcagagaaaaatgtaggtatttctgttttaacttaactgtaaactactgaactaatttagcttaatcttttgaatacaagttcaatttatgaggccagtgactatcaaaaaatttaagctgaaaaataatgggaaaaccactcaactcaccaaaattctgtttttcattaaaatctgagtttgtgacaaat

SEQ ID NO: 989 - DNA, 21 bp, Homo sapiens
aggtgacgttggagagagaga

SEQ ID NO: 990 - DNA, 20 bp, Homo sapiens
ccaatcaaattgctcccttg

SEQ ID NO: 991 - DNA, 125 bp, Homo sapiens
cgggtaggtacagtcatcctgtacttcgatcccaaatcagtctctggagactacttatttatttatttatttatttatggacttctttctttcaagcgttcgaactcatttccaccacaagaggg

SEQ ID NO: 992 - DNA, 21 bp, Homo sapiens
cgggtaggtacagtcatcctg

SEQ ID NO: 993 - DNA, 20 bp, Homo sapiens
ccctcttgtggtggaaatga

SEQ ID NO: 994 - DNA, 209 bp, Homo sapiens
tatcttgactgcactgtggggatgcgggatggagctgcctttcgagacacccctgagggtaggggcctgggacacaagtcataagtggcttcagaagttgtggccttgagcttacagggtctggaagctataagggtgtgtgtgtgtgtgtgtgtgtgtgtgtgtgtgtgtcaggaagttctatacagtgcctctaaggaagtcacatg

SEQ ID NO: 995 - DNA, 24 bp, Homo sapiens
catgtaacttccttagaggcactg

SEQ ID NO: 996 - DNA, 20 bp, Homo sapiens
tatcttgactgcactgtggg

SEQ ID NO: 997 - DNA, 255 bp, Homo sapiens
acatggttgtggagggttgtgggtgtcgctagtacagcaaaattaaatacataaatatatatatatatatatattttagaaaaaagaaaaaaacaaacaaacaaaaaaaccccaccccagttgacactttaatatttcccaatgaagactttatttatggaatggaatggaaaaaaaaacagctattttgaaaatatatttatatctacgaaaagaagttgggaaaacaaatattttaatcagagaattattcct

SEQ ID NO: 998 - DNA, 20 bp, Homo sapiens
acatggttgtggagggttgt

SEQ ID NO: 999 - DNA, 20 bp, Homo sapiens
tacttcatgtgctggggttg

SEQ ID NO: 1000 - DNA, 255 bp, Homo sapiens
gtgccacagcctccttatctgttctaaggcaaaaggaacatagccttgcagtcaagagcaattaaagcctatagtaattgtggcatagattcataatccaagaaaagttaagaatgtcctggcgccatttctcttctgttttatatttatttatttatttatttatttatttttgtgcagggaacgggtagggtgaaatgggggataagatgcaggtctgctcagcttgggtaaattgcttttcctctttcctcc

SEQ ID NO: 1001 - DNA, 20 bp, Homo sapiens
gtgccacagcctccttatct

SEQ ID NO: 1002 - DNA, 20 bp, Homo sapiens
ctgccctggagaataccaaa

SEQ ID NO: 1003 - DNA, 249 bp, Homo sapiens
tctcctctgcagccagaaacagtgattcatttgattttaatttttaacattagcatttttctcatgactcactgataatttaatatggaatttaaaaggttgaaacagttatttactttttaggaaattaaaacattccctaggtggatatatattttccctctgaaacagaagatgtactcgtggttgattaaaaaaaaaagaaagaaagaaagggaggagtgggcatttctctcacccttcccttcc

SEQ ID NO: 1004 - DNA, 20 bp, Homo sapiens
tctcctctgcagccagaaac

SEQ ID NO: 1005 - DNA, 20 bp, Homo sapiens
ggaagggaagggtgagagaa

SEQ ID NO: 1006 - DNA, 255 bp, Homo sapiens
tggaagaggatggcttgatttgaaacagagtgcataggaaaagcctcatccataaatgttgtggaaaagaatatgatctcatggcaaacaaataagaaaggctatcatgacaactagcttgaagctgtaattctgataaggcataataataataataataacagagagagacagggaatgatatggctgtagtgctctttgataagctccaagatatctctaggcttctgaaaaggtaagtgtctgtgcaaagct

SEQ ID NO: 1007 - DNA, 20 bp, Homo sapiens
tggaagaggatggcttgatt

SEQ ID NO: 1008 - DNA, 21 bp, Homo sapiens
tccagtctctcctgagtgcat

SEQ ID NO: 1009 - DNA, 255 bp, Homo sapiens
ctaggtaaattcaaccaatcaaaaaacaggcaaatcaaatacaaaaaaatttatattgtatcaaaaatttgtgtgtgtgtgtgtgtgtgtgtgtgtgtgtgtgtgtatgccagtcttgccatgaacctcttggtcagtattgaaataatgttccagaaacaaggctacttttgttgagtaaaaaatcaacacaaatgccctgtcctttgaaaatgttagatgcatcttatactgtgtattcctaaacagttgcaa

SEQ ID NO: 1010 - DNA, 20 bp, Homo sapiens
ctaggtaaattcaaccaatc

SEQ ID NO: 1011 - DNA, 20 bp, Homo sapiens
ttgcaactgtttaggaatac

SEQ ID NO: 1012 - DNA, 122 bp, Homo sapiens
tttattgaagcagcttgggtttttttttttttttttgattggttaactgacttattcatcccacaaatctttattaaacacttcatgtgccagtaaataaaaaaagatgatgaagccatgcc

SEQ ID NO: 1013 - DNA, 22 bp, Homo sapiens
tttattgaagcagcttgggttt

SEQ ID NO: 1014 - DNA, 20 bp, Homo sapiens
ggcatggcttcatcatcttt

SEQ ID NO: 1015 - DNA, 255 bp, Homo sapiens
gggttgtttgccaatatgaaagaaagagagatatcactgttttttttttctttttttttctttgtttgtttgtttgtttgtttttttgttaagctggcagctgctgtagtcggattcactggcattgactgccacctggttggggaggagtcctatatctttgcagattccatctcagagatgagacattgtccccctgtaattgcctttgtgtaatttcaggactagcatcatgatagatactatatggcagag

SEQ ID NO: 1016 - DNA, 21 bp, Homo sapiens
gggttgtttgccaatatgaaa

SEQ ID NO: 1017 - DNA, 20 bp, Homo sapiens
ccagctgagctttcctcaaa

(19) United States
(12) Patent Application Publication
(10) Pub. No.: US 2006/0057612 A1
(43) Pub. Date: Mar. 16, 2006

(54) Methods for diagnosing osteoporosis or a susceptibility to osteoporosis based on haplotype association

(76) Inventors: Unnur Styrkarsdottir, Reykjavik (IS); Jean-Baptiste Cazier, Reykjavik (IS); Jeffrey R. Gulcher, Lake Barrington, IL (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/185,033

(22) Filed: Jul. 18, 2005

Related U.S. Application Data
(63) Continuation-in-part of application No. PCT/US04/00991, filed on Jan. 15, 2004.
(63) Continuation-in-part of application No. PCT/US04/00990, filed on Jan. 15, 2004.
(63) Continuation of application No. 10/346,723, filed on Jan. 16, 2003.
Application No. 10/346,723 is a continuation-in-part of application No. 09/952,360, filed on Sep. 13, 2001, now abandoned.
Application No. 10/346,723 is a continuation-in-part of application No. PCT/IB01/01667, filed on Sep. 12, 2001.
Application No. PCT/IB01/01667 is a continuation-in-part of application No. 09/661,887, filed on Sep. 14, 2000, now Pat. No. 6,630,304.
(60) Provisional application No. 60/440,899, filed on Jan. 16, 2003.
(60) Provisional application No. 60/450,652, filed on Feb. 27, 2003.

(51) Int. Cl. C12Q 1/68; C12P 21/06

(52) U.S. Cl. 435/6; 435/69.1

(57) ABSTRACT

Methods for diagnosis of osteoporosis or a susceptibility to osteoporosis based on detection of at risk haplotypes associated with BMP2 are disclosed.

### RELATED APPLICATIONS

This application continuation-in-part of International Application No. PCT/US2004/000991, which designated the United States and was filed Jan. 15, 2004, published in English, which claims the benefit of U.S. Provisional Application No. 60/440,899, filed on Jan. 16, 2003, and claims the benefit of U.S. Provisional Application No. 60/450,652, filed on Feb. 27, 2003. This application is also a continuation-in-part of International Application No. PCT/US2004/000990, which designated the United States and was filed Jan. 15, 2004, published in English, which is a continuation of and claims priority to U.S. application Ser. No. 10/346,723, filed Jan. 16, 2003, which is a continuation-in-part of U.S. application Ser. No. 09/952,360, filed Sep. 13, 2001, and which is also a continuation-in-part and claims priority to International Application No. PCT/IB01/01667, which designated the United States and was filed on Sep. 12, 2001, published in English, which is a continuation-in-part of U.S. application Ser. No. 09/661,887, filed Sep. 14, 2000. The entire teachings of the above applications are incorporated herein by reference.

### INCORPORATION BY REFERENCE OF MATERIAL ON COMPACT DISK

This application incorporates by reference the Sequence Listing contained on the two compact disks (Copy 1 and Copy 2) filed concurrently herewith containing the following file:
a) File name: 2345.2052-003 SEQ. LIST.txt; created Jul. 18, 2005, 28 KB in size.

### BACKGROUND OF THE INVENTION

Osteoporosis is a debilitating disease characterized by low bone mass and deterioration of bone tissue, as defined by decreased bone mineral density (BMD). A direct result of the experienced microarchitectural deterioration is susceptibility to fractures and skeletal fragility, ultimately causing high mortality, morbidity and medical expenses worldwide. Postmenopausal woman are at greater risk than others because the estrogen deficiency and corresponding decrease in bone mass experienced during menopause increase both the probability of osteoporotic fracture and the number of potential fracture sites. However, aging women are not the only demographic group at risk. Young women who are malnourished, amenorrheic, or insufficiently active are at risk of inhibiting bone mass development at an early age. Furthermore, androgens play a role in the gain of bone mass during puberty, so elderly or hypogonadal men face the risk of osteoporosis if their bones were insufficiently developed.

The need to find a cure for this disease is complicated by the fact that there are many contributing factors that lead to osteoporosis. Nutrition (particularly calcium, vitamin D and vitamin K intake), hormone levels, age, sex, race, body weight, activity level, and genetic factors all influence the variance seen in bone mineral density among individuals. Currently, the drugs approved to treat osteoporosis act as inhibitors of bone reabsorption. Treatment regimens include methods such as hormone replacement therapy (HRT), the use of selective estrogen receptor modulators, calcitonin, and biophosphonates. However, these treatments may not individually reduce risk with consistent results. Moreover, while some therapies improve BMD when co-administered, others show no improvement or even loss of efficacy when used in combination.

Clearly, as life expectancy increases and health and economic concerns of osteoporosis grow, a solution for the risks associated with this late-onset disease is in great demand. Early diagnosis of the disease or detection of a susceptibility to the disease is therefore desirable.

### SUMMARY OF THE INVENTION

As described herein, it has been discovered that particular combinations of genetic markers (“haplotypes”), are present at a higher than expected frequency in patients with phenotypes associated with osteoporosis and a susceptibility to osteoporosis. The markers that are included in the haplotypes described herein are associated with the genomic region that directs expression of the human bone morphogenetic protein 2 (BMP2).

In one embodiment, the invention is directed to a method of diagnosing osteoporosis or a susceptibility to osteoporosis in an individual, comprising detecting the presence or absence of an at-risk haplotype, comprising a haplotype selected from the group consisting of: haplotype I, haplotype II, haplotype a, haplotype b, haplotype c, haplotype d and combinations thereof; wherein the presence of the haplotype is indicative of osteoporosis or a susceptibility to osteoporosis. In a particular embodiment, the invention is directed to assaying for the presence of a first nucleic acid molecule in a sample, comprising contacting said sample with a second nucleic acid molecule comprising the one or more haplotypes described herein. In one embodiment, determining the presence or absence of the haplotype comprises enzymatic amplification of nucleic acid from the individual. In a particular embodiment, determining the presence or absence of the haplotype further comprises electrophoretic analysis. For example, in one embodiment, determining the presence or absence of the haplotype comprises restriction fragment length polymorphism analysis. In another embodiment, determining the presence or absence of the haplotype comprises sequence analysis.

In another embodiment, the invention is directed to a method of diagnosing osteoporosis or a susceptibility to osteoporosis in an individual, comprising detecting the presence or absence of an at-risk haplotype comprising haplotype I, wherein the presence of the haplotype is indicative of osteoporosis or a susceptibility to osteoporosis. In a particular embodiment, determining the presence or absence of the haplotype comprises enzymatic amplification of nucleic acid from the individual. In a particular embodiment, determining the presence or absence of the haplotype further comprises electrophoretic analysis. For example, in one embodiment, determining the presence or absence of the haplotype comprises restriction fragment length polymorphism analysis. In another embodiment, determining the presence or absence of the haplotype comprises sequence analysis.

In another embodiment, the invention is directed to a method of diagnosing osteoporosis or a susceptibility to osteoporosis in an individual, comprising detecting the presence or absence of an at-risk haplotype comprising haplotype II, wherein the presence of the haplotype is indicative of osteoporosis or a susceptibility to osteoporosis. In a particular embodiment, determining the presence or absence of the haplotype comprises enzymatic amplification of nucleic acid from the individual. In a particular embodiment, determining the presence or absence of the haplotype further comprises electrophoretic analysis. For example, in one embodiment, determining the presence or absence of the haplotype comprises restriction fragment length polymorphism analysis. In another embodiment, determining the presence or absence of the haplotype comprises sequence analysis.

In another embodiment, the invention is directed to a kit for assaying a sample for the presence of a haplotype associated with osteoporosis, wherein the haplotype comprises two or more specific alleles, and wherein the kit comprises one or more nucleic acids capable of detecting the presence or absence of two or more of the specific alleles, thereby indicating the presence or absence of the haplotype in the sample. In a particular embodiment, the nucleic acid comprises a contiguous nucleotide sequence that is completely complementary to a region comprising specific allele of the haplotype.

In another embodiment, the invention is directed to a reagent kit for assaying a sample for the presence of a haplotype associated with osteoporosis, wherein the haplotype comprises two or more specific alleles, comprising in separate containers: a) one or more labeled nucleic acids capable of detecting one or more specific alleles of the haplotype; and b) reagents for detection of said label. In a particular embodiment, the labeled nucleic acid comprises a contiguous nucleotide sequence that is completely complementary to a region comprising specific allele of the haplotype.

In yet another embodiment, the invention is directed to a reagent kit for assaying a sample for the presence of a haplotype associated with osteoporosis, wherein the haplotype comprises two or more specific alleles, wherein the kit comprises one or more nucleic acids comprising a nucleotide sequence that is at least partially complementary to a part of the nucleotide sequence of the BMP2 gene, and wherein the nucleic acid is capable of acting as a primer for a primer extension reaction capable of detecting one or more of the specific alleles of the haplotype.

In another embodiment, the invention is directed to a method for the diagnosis and identification of susceptibility to osteoporosis in an individual, comprising: screening for an at-risk haplotype associated with BMP2 that is more frequently present in an individual susceptible to osteoporosis compared to an individual who is not susceptible to osteoporosis wherein the at-risk haplotype increases the risk significantly. In a particular embodiment, the significant increase is at least about 20%. In another embodiment, the significant increase is identified as an odds ratio of at least about 1.2.

In another embodiment, the invention is directed to a method for diagnosing a susceptibility to osteoporosis in an individual, comprising determining the presence or absence in the individual of a haplotype, comprising two or more alleles selected from the group consisting of: TSC0898956, B420, B8463, D20S846, TSC0191642, P4337, D20S892, B5048, B9082, D20S59, B7111/rs235764, B12845/rs15705, P9313, B10631, D35548, rs1116867, TSC0278787, D35548 and TSC0271643; wherein the presence of the haplotype is indicative of susceptibility to osteoporosis. In a particular embodiment, determining the presence or absence of the haplotype further comprises electrophoretic analysis. For example, in one embodiment, determining the presence or absence of the haplotype comprises restriction fragment length polymorphism analysis. In another embodiment, determining the presence or absence of the haplotype comprises sequence analysis.

In yet another embodiment, the invention is directed to a method for diagnosing a susceptibility to osteoporosis in an individual, comprising obtaining a nucleic acid sample from the individual; and analyzing the nucleic acid sample for the presence or absence of a haplotype comprising two or more alleles selected from the group consisting of: TSC0898956, B420, B8463, D20S846, TSC0191642, P4337, D20S892, B5048, B9082, D20S59, B7111/rs235764, B12845/rs15705, P9313, B10631, D35548, rs1116867, TSC0278787, D35548 and TSC0271643, wherein the presence of the haplotype is indicative of susceptibility to osteoporosis. In a particular embodiment, the alleles are selected from the group consisting of: TSC0898956, B420, B8463, D20S846 and TSC0191642. In a particular embodiment, the alleles are selected from the group consisting of: P4337, D20S892, B5048, B9082 and D20S59. In a different embodiment, the haplotype comprises B7111/rs235764 and B12845/rs15705. In a particular embodiment, the alleles are selected from the group consisting of: P9313, B10631 and D35548. In a particular embodiment, the alleles are selected from the group consisting of: rs1116867, TSC0278787 and D35548. In another embodiment, the alleles are selected from the group consisting of: TSC0271643, P9313 and B7111.

In another embodiment, the invention is directed to a method of diagnosing osteoporosis or a susceptibility to osteoporosis in an individual, comprising detecting the presence or absence of at least one at-risk haplotype comprising a haplotype selected from the group consisting of: haplotype G, haplotype V, and combinations thereof, wherein the presence of the haplotype is indicative of osteoporosis or a susceptibility to osteoporosis. In a particular embodiment, the invention is directed to assaying for the presence of a first nucleic acid molecule in a sample, comprising contacting said sample with a second nucleic acid molecule comprising the one or more haplotypes described herein. In a particular embodiment, determining the presence or absence of the haplotype comprises enzymatic amplification of nucleic acid from the individual, optionally further comprising electrophoretic analysis. In other embodiments, determining the presence or absence of the haplotype comprises restriction fragment length polymorphism analysis or sequence analysis.

In another embodiment, the invention is directed to a method for the diagnosis and identification of susceptibility to osteoporosis in an individual, comprising: screening for haplotype G or haplotype V, wherein the haplotype is more frequently present in an individual susceptible to osteoporosis compared to an individual who is not susceptible to osteoporosis, and wherein the at-risk haplotype increases the risk significantly. In a particular embodiment, the significant increase is at least about 20%. In a particular embodiment, the significant increase is identified as an odds ratio of at least about 1.2.

In another embodiment, the invention is directed to a method for diagnosing a susceptibility to osteoporosis in an individual, comprising determining the presence or absence in the individual at least one haplotype comprising one or more markers selected from the group consisting of: SG20S405, SG20S407, SG20S381, SG20S171, SG20S174, SG20S195 and D20S846, wherein the presence of the haplotype is indicative of susceptibility to osteoporosis. In a particular embodiment, determining the presence or absence of the haplotype comprises enzymatic amplification of nucleic acid from the individual, optionally further comprising electrophoretic analysis. In other embodiments, determining the presence or absence of the haplotype comprises restriction fragment length polymorphism analysis or sequence analysis.

In another embodiment, the invention is directed to a method for diagnosing a susceptibility to osteoporosis in an individual, comprising obtaining a nucleic acid sample from the individual; and analyzing the nucleic acid sample for the presence or absence of a haplotype comprising one or more alleles selected from the group consisting of: SG20S405, SG20S407, SG20S381, SG20S171, SG20S174, SG20S195 and D20S846, wherein the presence of the haplotype is indicative of susceptibility to osteoporosis. In a particular embodiment, the haplotype comprises one or more alleles selected from the group consisting of: SG20S405, SG20S407 and SG20S381. In another embodiment, the haplotype comprises one or more alleles selected from the group consisting of: SG20S174, SG20S195 and D20S846.

In another embodiment, the invention is directed to a method of diagnosing a susceptibility to osteoporosis in an individual, comprising detecting at least one polymorphism in a human BMP2 gene of SEQ ID NO: 1, wherein the polymorphism is selected from the group consisting of those listed in FIGS. 9.1 through 9.227. In a particular embodiment, the polymorphism is detected in a sample from a source selected from the group consisting of: blood, serum, cells and tissue.

In another embodiment, the invention is directed to an isolated nucleic acid molecule comprising the nucleic acid of SEQ ID NO:1 with one or more of the nucleic acid changes selected from the group consisting of those listed in FIGS. 12.1 through 12.13 and 13.

### BRIEF DESCRIPTION OF THE DRAWINGS

FIG. 1 is a tabular presentation of haplotype association data for haplotypes a, b and c for various phenotypes (as indicated, including BMP from spine and hip, osteoporotic fracture, weight corrected BMD). Data are also presented for pre- and post-menopausal patients.

FIG. 2 is a tabular presentation of haplotype association data for haplotype I and haplotype II. Data are presented for fracture and weight corrected BMD for hip and spine.

FIG. 3 is a tabular presentation of haplotype d for various phenotypes (as indicated, including BMD from spine and hip, osteoporotic fracture, weight corrected BMD). The BMD values represent the lowest 10^{th} percentile in all cases. Data are also presented for pre- and post-menopausal patients.

FIG. 4 is a schematic summary of splice site variants detected in the BMP2 gene.

FIG. 5 is a listing of clone sequences shown on the UCSC Genome Browser on Human May 2004_hg17_Build35 Assembly.

FIG. 6 is a schematic close up view of the clone sequences at the 3′end of the BMP2 gene.

FIG. 7 is an alignment showing the sequences of splice variants and a consensus sequence.

FIGS. 8A-E are a listing of primer and clone sequences. FIG. 8A shows primers used to amplify BMP2 exons. FIGS. 8B-E list clone sequences.

FIG. 9.1-9.227 are a listing of SNPs detected in the BMP2 gene (see Example 3).

FIGS. 10.1-10.8 are a listing of microsatellite markers according to NCBI_build3.

FIGS. 11A-C are a listing of BMP2 microsatellite markers.

FIGS. 12.1-12.13 are a listing of BMP2-associated SNPs.

FIG. 13 is a data table showing the relationship between markers and osteoporosis-related phenotypes.

FIGS. 14A and 14B show markers included in haplotypes G (“hapG”) and V (“hapV”) and their association with fracture.

### DETAILED DESCRIPTION OF THE INVENTION

As described herein, Applicant has completed linkage analysis between osteoporosis phenotypes and particular combinations of genetic markers (“haplotypes”) associated with the genomic region, located on chromosome 20, that directs expression of the human bone morphogenetic protein 2 (BMP2). The results shown here represent the first demonstration of haplotypes used to indicate osteoporosis or a susceptibility to osteoporosis. Based on the linkage studies conducted, Applicant has discovered a direct relationship between the BMP2-associated haplotypes and osteoporosis. In particular, it has been discovered that particular haplotypes appear at higher than expected frequencies in patients with phenotypes associated with osteoporosis and a susceptibility to osteoporosis. Methods for the diagnosis of osteoporosis based on this association, in combination with, for example, bone turnover marker assays (e.g., bone scans), are described herein. Additionally, methods based on the detection of at least one haplotype described herein is diagnostic of a susceptibility to osteoporosis.

Diagnostic and Screening Assays of the Invention

The present invention pertains to methods of diagnosing or aiding in the diagnosis of osteoporosis or a susceptibility to osteoporosis by detecting particular genetic markers that appear more frequently in individuals with osteoporosis or who are susceptible to osteoporosis. Diagnostic assays can be designed for assessing BMP2. Such assays can be used alone or in combination with other assays, e.g., bone turnover marker assays (e.g., bone scans). Combinations of genetic markers are referred to herein as “haplotypes,” and the present invention describes methods whereby detection of particular haplotypes is indicative of osteoporosis or a susceptibility to osteoporosis. The detection of the particular genetic markers that make up the particular haplotypes can be performed by a variety of methods described herein and known in the art. For example, genetic markers can be detected at the nucleic acid level, e.g., by direct sequencing or at the amino acid level if the genetic marker affects the coding sequence of BMP2, e.g., by immunoassays based on antibodies that recognize the BMP2 protein or a particular BMP2 variant protein.

In one embodiment, the assays are used in the context of a biological sample (e.g., blood, serum, cells, tissue) to thereby determine whether an individual is afflicted with osteoporosis, or is at risk for (has a predisposition for or a susceptibility to) developing osteoporosis. The invention also provides for prognostic (or predictive) assays for determining whether an individual is susceptible to developing osteoporosis. For example, variations in a nucleic acid sequence can be assayed in a biological sample. Such assays can be used for prognostic or predictive purposes to thereby allow for the prophylactic treatment of an individual prior to the onset of symptoms associated with osteoporosis.

The haplotypes and markers disclosed herein are in “linkage disequilibrium” with the BMP2 gene and, likewise, osteoporosis and BMP2-associated phenotypes (e.g., loss of bone marrow density and susceptibility to fracture). “Linkage” refers to a higher than expected statistical association of genotypes and/or phenotypes with each other. “Linkage Disequilibrium” (LD) refers to a non-random assortment of two genetic elements. For example, if a particular genetic element (e.g., an allele at a polymorphic site) occurs in a population at a frequency of 0.25 and another occurs at a frequency of 0.25, then the predicted occurrence of a person's having both elements is 0.125, assuming a random distribution of the elements. However, if it is discovered that the two elements occur together at a frequency higher than 0.125, then the elements are said to be in LD since they tend to be inherited together at a higher frequency than what their independent allele frequencies would predict. Roughly speaking, LD is generally correlated with the frequency of recombination events between the two elements. Allele frequencies can be determined in a population, for example, by genotyping individuals in a population and determining the occurrence of each allele in the population. For populations of diploid individuals, e.g., human populations, individuals will typically have two alleles for each genetic element (e.g., a marker or gene).

Many different measures have been proposed for assessing the strength of linkage disequilibrium (LD). Most capture the strength of association between pairs of biallelic sites. Two important pairwise measures of LD are r² (sometimes denoted Δ²) and |D′|. Both measures range from 0 (no disequilibrium) to 1 (‘complete’ disequilibrium), but their interpretation is slightly different. |D′| is defined in such a way that it is equal to 1 if just two or three of the possible haplotypes are present, and it is <1 if all four possible haplotypes are present. So, a value of |D′| that is <1 indicates that historical recombination has occurred between two sites (recurrent mutation can also cause |D′| to be <1, but for single nucleotide polymorphisms (SNPs) this is usually regarded as being less likely than recombination).

The measure r² represents the statistical correlation between two sites, and takes the value of 1 if only two haplotypes are present. It is arguably the most relevant measure for association mapping, because there is a simple inverse relationship between r² and the sample size required to detect association between susceptibility loci and SNPs. These measures are defined for pairs of sites, but for some applications a determination of how strong LD is across an entire region that contains many polymorphic sites might be desirable (e.g., testing whether the strength of LD differs significantly among loci or across populations, or whether there is more or less LD in a region than predicted under a particular model). Measuring LD across a region is not straightforward, but one approach is to use the measure r, which was developed in population genetics. Roughly speaking, r measures how much recombination would be required under a particular population model to generate the LD that is seen in the data. This type of method can potentially also provide a statistically rigorous approach to the problem of determining whether LD data provide evidence for the presence of recombination hotspots. For the methods described herein, a significant r² value can be 0.3, 0.4, 0.5, 0.6, 0.7, 0.8, 0.9 or 1.0. Thus, LD represents a correlation between alleles of distinct markers. It is measured by correlation coefficient or |D′| (r² up to 1.0 and |D′| up to 1.0).

The invention pertains to markers identified in a “haplotype block” or “LD block” (specific instances of which are disclosed herein, see Exemplification). These blocks are defined either by their physical proximity to a genetic element, e.g., the BMP2 gene, or by their “genetic distance” from the element. Other blocks would be apparent to one of skill in the art as genetic regions in LD with BMP2. Markers and haplotypes identified in these blocks, because of their association with BMP2, are encompassed by the invention. One of skill in the art will appreciate regions of chromosomes that recombine infrequently and regions of chromosomes that are “hotspots”, e.g., exhibiting frequent recombination events, are descriptive of LD blocks. Regions of infrequent recombination events bounded by hotspots will form a block that will be maintained during cell division. Thus, identification of a marker associated with a phenotype, wherein the marker is contained within an LD block, identifies the block as associated with the phenotype. Any marker identified within the block can therefore be used to indicate the phenotype.

Additional markers that are in LD with the BMP2 markers or haplotypes are referred to herein as “surrogate” markers. Such a surrogate is a marker for another marker or another surrogate marker. Surrogate markers are themselves markers and are indicative of the presence of another marker, which is in turn indicative of either another marker or an associated phenotype.

Diagnostic Assays

In one embodiment of the invention, diagnosis of a susceptibility to osteoporosis is made by detecting a haplotype associated with BMP2 as described herein. The BMP2-associated haplotypes describe a set of genetic markers associated with BMP2. In a certain embodiment, the haplotype can comprise one or more markers, two or more markers, three or more markers, four or more markers, or five or more markers. The genetic markers are particular “alleles” at “polymorphic sites” associated with BMP2. A nucleotide position at which more than one sequence is possible in a population (either a natural population or a synthetic population, e.g., a library of synthetic molecules), is referred to herein as a “polymorphic site”. Where a polymorphic site is a single nucleotide in length, the site is referred to as a single nucleotide polymorphism (“SNP”). For example, if at a particular chromosomal location, one member of a population has an adenine and another member of the population has a thymine at the same position, then this position is a polymorphic site, and, more specifically, the polymorphic site is a SNP. Polymorphic sites can allow for differences in sequences based on substitutions, insertions or deletions. Each version of the sequence with respect to the polymorphic site is referred to herein as an “allele” of the polymorphic site. Thus, in the previous example, the SNP allows for both an adenine allele and a thymine allele.

Typically, a reference sequence is referred to for a particular sequence. Alleles that differ from the reference are referred to as “variant” alleles. For example, the reference BMP2 sequence is described herein by SEQ ID NO:1. The term, “variant BMP2”, as used herein, refers to a BMP2 sequence that differs from SEQ ID NO:1, but is otherwise substantially similar. The genetic markers that make up the haplotypes described herein are BMP2 variants. The variants of BMP2 that are used to determine the haplotypes disclosed herein of the present invention are associated with a susceptibility to a number of osteoporosis phenotypes.

Additional variants can include changes that affect a polypeptide, e.g., the BMP2 polypeptide. These sequence differences, when compared to a reference nucleotide sequence, can include the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of a reading frame; duplication of all or a part of a sequence; transposition; or a rearrangement of a nucleotide sequence. Such sequence changes alter the polypeptide encoded by a BMP2 nucleic acid. For example, if the change in the nucleic acid sequence causes a frame shift, the frame shift can result in a change in the encoded amino acids, and/or can result in the generation of a premature stop codon, causing generation of a truncated polypeptide. Alternatively, a polymorphism associated with a susceptibility to osteoporosis can be a synonymous change in one or more nucleotides (i.e., a change that does not result in a change in the BMP2 amino acid sequence). Such a polymorphism can, for example, alter splice sites, affect the stability or transport of mRNA, or otherwise affect the transcription or translation of the polypeptide. The polypeptide encoded by the reference nucleotide sequence is the “reference” polypeptide with a particular reference amino acid sequence, and polypeptides encoded by variant alleles are referred to as “variant” polypeptides with variant amino acid sequences.

Haplotypes are a combination of genetic markers, e.g., particular alleles at polymorphic sites. The haplotypes described herein are associated with osteoporosis and/or a susceptibility to osteoporosis. Therefore, detection of the presence or absence of the haplotypes herein is indicative of osteoporosis, a susceptibility to osteoporosis or a lack thereof. Detection of the presence or absence of these haplotypes, therefore, is necessary for the purposes of the invention, in order to detect osteoporosis or a susceptibility to osteoporosis. The haplotypes described herein are a combination of various genetic markers, e.g., SNPs and microsatellites. Therefore, detecting haplotypes can be accomplished by methods known in the art for detecting sequences at polymorphic sites.

In a first method of diagnosing a susceptibility to osteoporosis, hybridization methods, such as Southern analysis, Northern analysis, or in situ hybridizations, can be used (see Current Protocols in Molecular Biology, Ausubel, F. et al., eds., John Wiley & Sons, including all supplements through 1999). For example, a biological sample from a test subject (a “test sample”) of genomic DNA, RNA, or cDNA, is obtained from an individual suspected of having, being susceptible to or predisposed for, or carrying a defect for, osteoporosis (the “test individual”). The individual can be an adult, child, or fetus. The test sample can be from any source that contains genomic DNA, such as a blood sample, sample of amniotic fluid, sample of cerebrospinal fluid, or tissue sample from skin, muscle, buccal or conjunctival mucosa, placenta, gastrointestinal tract or other organs. A test sample of DNA from fetal cells or tissue can be obtained by appropriate methods, such as by amniocentesis or chorionic villus sampling. The DNA, RNA, or cDNA sample is then examined to determine whether a polymorphism in BMP2 is present. The presence of an allele of the haplotype can be indicated by sequence-specific hybridization of a nucleic acid probe specific for the particular allele. A sequence-specific probe can be directed to hybridize to genomic DNA, RNA, or cDNA. A “nucleic acid probe”, as used herein, can be a DNA probe or an RNA probe that hybridizes to a complementary sequence. One of skill in the art would know how to design such a probe such that sequence specific hybridization will occur only if a particular allele is present in a genomic sequence from a test sample.

To diagnose a susceptibility to osteoporosis, a hybridization sample is formed by contacting the test sample containing BMP2, with at least one nucleic acid probe. A non-limiting example of a probe for detecting mRNA or genomic DNA is a labeled nucleic acid probe capable of hybridizing to mRNA or genomic DNA sequences described herein. The nucleic acid probe can be, for example, a full-length nucleic acid molecule, or a portion thereof, such as an oligonucleotide of at least 15, 30, 50, 100, 250 or 500 nucleotides in length and sufficient to specifically hybridize under stringent conditions to appropriate mRNA or genomic DNA. For example, the nucleic acid probe can be all or a portion of SEQ ID NO:1, optionally comprising at least one allele contained in the haplotypes described herein, or the probe can be the complementary sequence of such a sequence. Other suitable probes for use in the diagnostic assays of the invention are described herein.

The hybridization sample is maintained under conditions that are sufficient to allow specific hybridization of the nucleic acid probe to BMP2. “Specific hybridization”, as used herein, indicates exact hybridization (e.g., with no mismatches). Specific hybridization can be performed under high stringency conditions or moderate stringency conditions (see below). In one embodiment, the hybridization conditions for specific hybridization are high stringency.

Specific hybridization, if present, is then detected using standard methods. If specific hybridization occurs between the nucleic acid probe and BMP2 in the test sample, then the sample contains the allele that is present in the nucleic acid probe. The process can be repeated for the other markers that make up the haplotype, or multiple probes can be used concurrently to detect more than one marker at a time. Detection of the particular markers of the haplotype in the sample is indicative that the source of the sample has the particular haplotype and therefore has osteoporosis or a susceptibility to osteoporosis.

In another hybridization method, Northern analysis (see Current Protocols in Molecular Biology, Ausubel, F. et al., eds., John Wiley & Sons, supra) is used to identify the presence of a polymorphism associated with a susceptibility to osteoporosis. For Northern analysis, a test sample of RNA is obtained from the individual by appropriate means. Specific hybridization of a nucleic acid probe, as described above, to RNA from the individual is indicative of a particular allele complementary to the probe.

For representative examples of use of nucleic acid probes, see, for example, U.S. Pat. Nos. 5,288,611 and 4,851,330.

Alternatively, a peptide nucleic acid (PNA) probe can be used instead of a nucleic acid probe in the hybridization methods described above. PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, Nielsen, P. et al., 1994. Bioconjug. Chem., 5:3-7). The PNA probe can be designed to specifically hybridize to a molecule in a sample suspected of containing one of the genetic markers of the haplotypes associated with a susceptibility to osteoporosis. Hybridization of the PNA probe is diagnostic for osteoporosis or a susceptibility to osteoporosis.

In one embodiment of the invention, diagnosis of osteoporosis or a susceptibility to osteoporosis associated with BMP2 or a haplotype associated with osteoporosis, can be made by expression analysis using quantitative PCR (kinetic thermal cycling). In one embodiment, the diagnosis of osteoporosis is made by detecting at least one BMP2-associated allele and in combination with a bone turnover marker assay (e.g., bone scans). This technique can, for example, utilize commercially available technologies such as TaqMan® (Applied Biosystems, Foster City, Calif.), to allow the identification of polymorphisms and haplotypes. The technique can assess the presence of an alteration in the expression or composition of the polypeptide encoded by BMP2 or splicing variants. Further, the expression of the variants can be quantified as physically or functionally different.

In another method of the invention, analysis by restriction digestion can be used to detect a particular allele if the allele results in the creation or elimination of a restriction site relative to a reference sequence. A test sample containing genomic DNA is obtained from the individual. Polymerase chain reaction (PCR) can be used to amplify the genomic BMP2 region (including flanking sequences if necessary) in the test sample from the test individual. RFLP analysis is conducted as described (see Current Protocols in Molecular Biology, supra). The digestion pattern of the relevant DNA fragment indicates the presence or absence of the particular allele in the sample.

Sequence analysis can also be used to detect specific alleles at polymorphic sites associated with BMP2. A test sample of DNA or RNA is obtained from the test individual. PCR or other appropriate methods can be used to amplify BMP2 and/or its flanking sequences, if desired. The presence of a specific allele is thus detected directly by sequencing the polymorphic site of the genomic DNA in the sample.

Allele-specific oligonucleotides can also be used to detect the presence of a particular allele at a polymorphic site associated with BMP2, through the use of dot-blot hybridization of amplified oligonucleotides with allele-specific oligonucleotide (ASO) probes (see, for example, Saiki, R. et al., 1986. Nature, 324:163-166). An “allele-specific oligonucleotide” (also referred to herein as an “allele-specific oligonucleotide probe”) is an oligonucleotide of approximately 10-50 base pairs or approximately 15-30 base pairs, that specifically hybridizes to BMP2, and that contains a specific allele at a polymorphic site as indicated by the haplotypes described herein. An allele-specific oligonucleotide probe that is specific for particular polymorphisms in BMP2 can be prepared, using standard methods (see Current Protocols in Molecular Biology, supra). PCR can be used to amplify all or a fragment of BMP2, as well as genomic flanking sequences. The DNA containing the amplified BMP2 (or fragment of the gene) is dot-blotted, using standard methods (see Current Protocols in Molecular Biology, supra), and the blot is contacted with the oligonucleotide probe. The presence of specific hybridization of the probe to the amplified BMP2 is then detected. Specific hybridization of an allele-specific oligonucleotide probe to DNA from the individual is indicative of a specific allele at a polymorphic site associated with BMP2.

An allele-specific primer hybridizes to a site on target DNA overlapping a polymorphic site and only primes amplification of an allelic form to which the primer exhibits perfect complementarity (Gibbs, R. et al., 1989. Nucleic Acids Res., 17:2437-2448). This primer is used in conjunction with a second primer, which hybridizes at a distal site on the opposite strand. Amplification proceeds from the two primers, resulting in a detectable product, which indicates the particular allelic form is present. A control is usually performed with a second pair of primers, one of which shows a single base mismatch at the polymorphic site and the other of which exhibits perfect complementarity to a distal site. The single-base mismatch prevents amplification and no detectable product is formed. The method works best when the mismatch is included in the 3′-most position of the oligonucleotide aligned with the polymorphism because this position is most destabilizing to elongation from the primer (see, e.g., WO 93/22456).

With the addition of such analogs as locked nucleic acids (LNAs), the size of primers and probes can be reduced to as few as 8 bases. LNAs are a novel class of bicyclic DNA analogs in which the 2′ and 4′ positions in the furanose ring are joined via an O-methylene (oxy-LNA), S-methylene (thio-LNA), or amino methylene (amino-LNA) moiety. Common to all of these LNA variants is an affinity toward complementary nucleic acids, which is by far the highest reported for a DNA analog. For example, particular all oxy-LNA nonamers have been shown to have melting temperatures of 64° C., and 74° C., when in complex with complementary DNA or RNA, respectively, as opposed to 28° C., for both DNA and RNA for the corresponding DNA nonamer. Substantial increases in Tₘ are also obtained when LNA monomers are used in combination with standard DNA or RNA monomers. For primers and probes, depending on where the LNA monomers are included (e.g., the 3′ end, the 5′end, or in the middle), the Tₘ could be increased considerably.

In another embodiment, arrays of oligonucleotide probes that are complementary to target nucleic acid sequence segments from an individual, can be used to identify polymorphisms in a BMP2 nucleic acid. For example, in one embodiment, an oligonucleotide array can be used. Oligonucleotide arrays typically comprise a plurality of different oligonucleotide probes that are coupled to a surface of a substrate in different known locations. These oligonucleotide arrays, also described as “Genechips™,” have been generally described in the art, for example, U.S. Pat. No. 5,143,854 and PCT patent publication Nos. WO 90/15070 and 92/10092. These arrays can generally be produced using mechanical synthesis methods or light directed synthesis methods that incorporate a combination of photolithographic methods and solid phase oligonucleotide synthesis methods (Fodor, S. et al., 1991. Science, 251:767-773; Pirrung et al., U.S. Pat. No. 5,143,854 (see also PCT Application No. WO 90/15070); and Fodor. S. et al., PCT Publication No. WO 92/10092 and U.S. Pat. No. 5,424,186, the entire teachings of each of which are incorporated by reference herein). Techniques for the synthesis of these arrays using mechanical synthesis methods are described in, e.g., U.S. Pat. No. 5,384,261; the entire teachings of which are incorporated by reference herein. In another example, linear arrays can be utilized.

Once an oligonucleotide array is prepared, a nucleic acid of interest is allowed to hybridize with the array. Detection of hybridization is a detection of a particular allele in the nucleic acid of interest. Hybridization and scanning are generally carried out by methods described herein and also in, e.g., published PCT Application Nos. WO 92/10092 and WO 95/11995, and U.S. Pat. No. 5,424,186, the entire teachings of which are incorporated by reference herein. In brief, a target nucleic acid sequence, which includes one or more previously identified polymorphic markers, is amplified by well known amplification techniques, e.g., PCR. Typically this involves the use of primer sequences that are complementary to the two strands of the target sequence, both upstream and downstream, from the polymorphic site. Asymmetric PCR techniques can also be used. Amplified target, generally incorporating a label, is then allowed to hybridize with the array under appropriate conditions that allow for sequence-specific hybridization. Upon completion of hybridization and washing of the array, the array is scanned to determine the position on the array to which the target sequence hybridizes. The hybridization data obtained from the scan is typically in the form of fluorescence intensities as a function of location on the array.

Although primarily described in terms of a single detection block, e.g., for detection of a single polymorphic site, arrays can include multiple detection blocks, and thus be capable of analyzing multiple, specific polymorphisms. In alternate arrangements, it will generally be understood that detection blocks can be grouped within a single array or in multiple, separate arrays so that varying, optimal conditions can be used during the hybridization of the target to the array. For example, it will often be desirable to provide for the detection of those polymorphisms that fall within G-C rich stretches of a genomic sequence, separately from those falling in A-T rich segments. This allows for the separate optimization of hybridization conditions for each situation.

Additional descriptions of use of oligonucleotide arrays for detection of polymorphisms can be found, for example, in U.S. Pat. Nos. 5,858,659 and 5,837,832, the entire teachings of which are incorporated by reference herein.

Other methods of nucleic acid analysis can be used to detect a particular allele at a polymorphic site associated with BMP2. Representative methods include, for example, direct manual sequencing (Church and Gilbert, 1988. Proc. Natl. Acad. Sci. USA, 81:1991-1995; Sanger, F. et al., 1977. Proc. Natl. Acad. Sci. USA, 74:5463-5467; Beavis et al. U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (Sheffield, V. et al., 1989. Proc. Natl. Acad. Sci. USA, 86:232-236), mobility shift analysis (Orita, M. et al., 1989. Proc. Natl. Acad. Sci. USA, 86:2766-2770), restriction enzyme analysis (Flavell, R. et al., 1978. Cell, 15:25-41; Geever, R. et al., 1981. Proc. Natl. Acad. Sci. USA, 78:5081-5085); heteroduplex analysis; chemical mismatch cleavage (CMC) (Cotton, R. et al., 1985. Proc. Natl. Acad. Sci. USA, 85:4397-4401); RNase protection assays (Myers, R. et al., 1985. Science, 230:1242-1246); use of polypeptides that recognize nucleotide mismatches, such as E. coli mutS protein; and allele-specific PCR.

In another embodiment of the invention, diagnosis of a susceptibility to osteoporosis can also be made by examining expression and/or composition of an BMP2 polypeptide in those instances where the genetic marker contained in a haplotype described herein results in a change in the expression of the polypeptide (e.g., an altered amino acid sequence or a change in expression levels). A variety of methods can be used to make such a detection, including enzyme linked immunosorbent assays (ELISA), Western blots, immunoprecipitations and immunofluorescence. A test sample from an individual is assessed for the presence of an alteration in the expression and/or an alteration in composition of the polypeptide encoded by BMP2. An alteration in expression of a polypeptide encoded by BMP2 can be, for example, an alteration in the quantitative polypeptide expression (i.e., the amount of polypeptide produced); an alteration in the composition of a polypeptide encoded by BMP2 is an alteration in the qualitative polypeptide expression (e.g., expression of a mutant BMP2 polypeptide or of a different splicing variant). In one embodiment, diagnosis of a susceptibility to osteoporosis is made by detecting a particular splicing variant encoded by BMP2, or a particular pattern of splicing variants.

Both such alterations (quantitative and qualitative) can also be present. An “alteration” in the polypeptide expression or composition, as used herein, refers to an alteration in expression or composition in a test sample, as compared to the expression or composition of polypeptide by BMP2 in a control sample. A control sample is a sample that corresponds to the test sample (e.g., is from the same type of cells), and is from an individual who is not affected by osteoporosis or a susceptibility to osteoporosis. Similarly, the presence of one or more different splicing variants in the test sample, or the presence of significantly different amounts of different splicing variants in the test sample, as compared with the control sample, is indicative of a susceptibility to osteoporosis. An alteration in the expression or composition of the polypeptide in the test sample, as compared with the control sample, can be indicative of a specific allele in the instance where the allele alters a splice site relative to the reference. Various means of examining expression or composition of the polypeptide encoded by BMP2 can be used, including spectroscopy, colorimetry, electrophoresis, isoelectric focusing, and immunoassays (e.g., David et al., U.S. Pat. No. 4,376,110) such as immunoblotting (see also Current Protocols in Molecular Biology, particularly chapter 10).

For example, in one embodiment, an antibody capable of binding to the polypeptide (e.g., as described above), e.g., an antibody with a detectable label, can be used. Antibodies can be polyclonal or monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

Western blot analysis, using an antibody as described above that specifically binds to a polypeptide encoded by a variant BMP2, or an antibody that specifically binds to a polypeptide encoded by a reference allele, can be used to identify the presence in a test sample of a polypeptide encoded by a variant BMP2 allele, or the absence in a test sample of a polypeptide encoded by the reference allele.

In one embodiment of this method, the level or amount of polypeptide encoded by BMP2 in a test sample is compared with the level or amount of the polypeptide encoded by BMP2 in a control sample. A level or amount of the polypeptide in the test sample that is higher or lower than the level or amount of the polypeptide in the control sample, such that the difference is statistically significant, is indicative of an alteration in the expression of the polypeptide encoded by BMP2, and is diagnostic for a particular allele responsible for causing the difference in expression. Alternatively, the composition of the polypeptide encoded by BMP2 in a test sample is compared with the composition of the polypeptide encoded by BMP2 in a control sample. In another embodiment, both the level or amount and the composition of the polypeptide can be assessed in the test sample and in the control sample.

Kits useful in the methods of diagnosis comprise components useful in any of the methods described herein, including for example, hybridization probes, restriction enzymes (e.g., for RFLP analysis), allele-specific oligonucleotides, antibodies which bind to altered or to non-altered (native) BMP2 polypeptide (e.g., to SEQ ID NO:2 and comprising at least one genetic marker included in the haplotypes described herein), means for amplification of nucleic acids comprising BMP2, or means for analyzing the nucleic acid sequence of BMP2 or for analyzing the amino acid sequence of an BMP2 polypeptide, etc. Additionally, kits can provide reagents for assays to be used in combination with the methods of the present invention, e.g., bone turnover marker assays (e.g., bone scans).

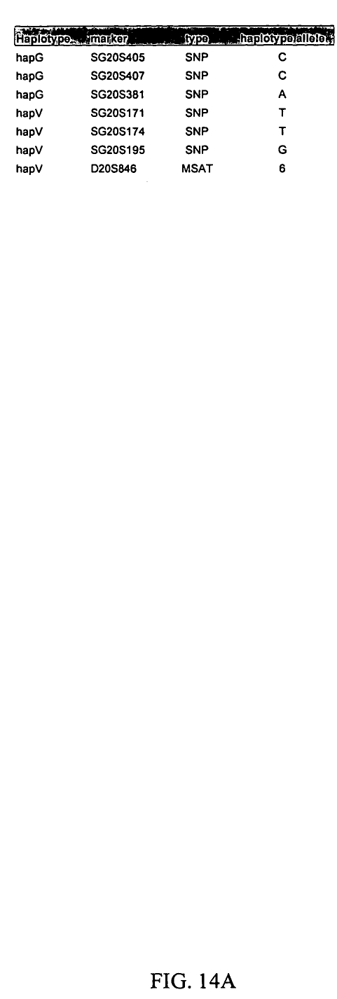

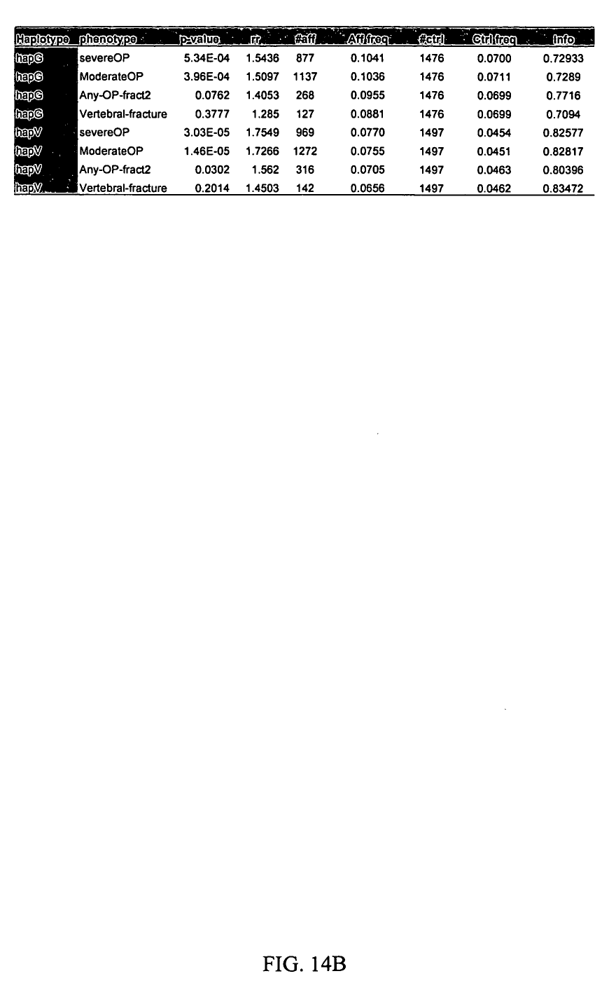

Kits (e.g., reagent kits) useful in the methods of diagnosis comprise components useful in any of the methods described herein, including for example, hybridization probes or primers as described herein (e.g., labeled probes or primers), reagents for detection of labeled molecules, restriction enzymes (e.g., for RFLP analysis), allele-specific oligonucleotides, antibodies that bind to altered or to non-altered (native) BMP2 polypeptide, means for amplification of nucleic acids comprising a BMP2, or means for analyzing the nucleic acid sequence of a BMP2 nucleic acid or for analyzing the amino acid sequence of a BMP2 polypeptide as described herein, etc. In one embodiment, the kit for diagnosing osteoporosis or a susceptibility to osteoporosis can comprise primers for nucleic acid amplification of a region in the BMP2 nucleic acid comprising an at-risk haplotype that is more frequently present in an individual having osteoporosis or is susceptible to osteoporosis. The primers can be designed using portions of the nucleic acids flanking SNPs that are indicative of osteoporosis. In a certain embodiment, the primers are designed to amplify regions of the BMP2 nucleic acid associated with an at-risk haplotype for osteoporosis, shown in Table 1 and FIGS. 14A and 14B, or more particularly haplotype I, haplotype II, haplotype a, haplotype b, haplotype c, haplotype d, hapG or hapV. Additionally, kits can provide reagents for assays to be used in combination with the methods of the present invention, e.g., bone turnover marker assays (e.g., bone scans).

Haplotype Screening

The invention further pertains to a method for the diagnosis and identification of susceptibility to osteoporosis in an individual, by identifying an at-risk haplotype in BMP2. In one embodiment, the at-risk haplotype is one that confers a significant risk of osteoporosis. In one embodiment, significance associated with a haplotype is measured by an odds ratio. In a further embodiment, the significance is measured by a percentage. In one embodiment, a significant risk is measured as an odds ratio of at least about 1.2, including by not limited to: 1.2, 1.3, 1.4, 1.5, 1.6, 1.7, 1.8, and 1.9. In a further embodiment, an odds ratio of at least 1.2 is significant. In a further embodiment, an odds ratio of at least about 1.5 is significant. In a further embodiment, a significant increase in risk is at least about 1.7 is significant. In a further embodiment, a significant increase in risk is at least about 20%, including but not limited to about 25%, 30%, 35%, 40%, 45%, 50%, 55%, 60%, 65%, 70%, 75%, 80%, 85%, 90%, 95% and 98%. In a further embodiment, a significant increase in risk is at least about 50%. It is understood however, that identifying whether a risk is medically significant may also depend on a variety of factors, including the specific disease, the haplotype, and often, environmental factors.

The invention also pertains to methods of diagnosing osteoporosis or a susceptibility to osteoporosis in an individual, comprising screening for an at-risk haplotype associated with the BMP2 nucleic acid that is more frequently present in an individual susceptible to osteoporosis (affected), compared to the frequency of its presence in a healthy individual (control), wherein the presence of the haplotype is indicative of osteoporosis or susceptibility to osteoporosis. Standard techniques for genotyping for the presence of SNPs and/or microsatellite markers that are associated with osteoporosis can be used, such as fluorescent based techniques (Chen, X. et al., 1999. Genome Res., 9:492-498), PCR, LCR, Nested PCR and other techniques for nucleic acid amplification. In one embodiment, the method comprises assessing in an individual the presence or frequency of a specific SNP allele or microsatellite allele associated with the BMP2 nucleic acid that are associated with osteoporosis, wherein an excess or higher frequency of the haplotype compared to a healthy control individual is indicative that the individual has osteoporosis or is susceptible to osteoporosis.

Haplotype analysis involves defining a candidate susceptibility locus using LOD scores. The defined regions are then ultra-fine mapped with microsatellite markers with an average spacing between markers of less than 100 kb. All usable microsatellite markers that found in public databases and mapped within that region can be used. In addition, microsatellite markers identified within the deCODE genetics sequence assembly of the human genome can be used.

The frequencies of haplotypes in the patient and the control groups using an expectation-maximization algorithm can be estimated (Dempster A. et al., 1977. J R. Stat. Soc. B, 39:1-389). An implementation of this algorithm that can handle missing genotypes and uncertainty with the phase can be used. Under the null hypothesis, the patients and the controls are assumed to have identical frequencies. Using a likelihood approach, an alternative hypothesis where a candidate at-risk-haplotype is allowed to have a higher frequency in patients than controls, while the ratios of the frequencies of other haplotypes are assumed to be the same in both groups is tested. Likelihoods are maximized separately under both hypotheses and a corresponding 1-df likelihood ratio statistics is used to evaluate the statistic significance.

To look for at-risk-haplotypes in the 1-lod drop, for example, association of all possible combinations of genotyped markers is studied, provided those markers span a practical region. The combined patient and control groups can be randomly divided into two sets, equal in size to the original group of patients and controls. The haplotype analysis is then repeated and the most significant p-value registered is determined. This randomization scheme can be repeated, for example, over 100 times to construct an empirical distribution of p-values.

It is possible to identify a physical linkage between a genetic locus associated with a trait of interest (e.g., disease) and polymorphic markers that are in physical or statistical proximity with the genetic locus responsible for the trait and co-segregate with it. Such analysis is useful for mapping a genetic locus associated with a phenotypic trait to a chromosomal position, and thereby cloning gene(s) responsible for the trait (Lander, E. and Botstein, D., 1986. Proc. Natl. Acad. Sci. USA, 83:7353-7357 (1986); Lander, E. and Green, P., 1987. Proc. Natl. Acad. Sci. USA, 84:2363-2367 (1987); Donis-Keller, H. et al., 1987. Cell, 51:319-337; Lander, E. and Botstein, D., 1989. Genetics, 121:185-199). Genes localized by linkage can be cloned by a process known as directional cloning (Wainwright, B., 1993. Med. J. Australia, 159:170-174; Collins, F., 1992. Nat. Genet., 1:3-6).

Linkage studies are typically performed on members of a family, such as the phenotype proband and his/her parents studied. Available members of the family are characterized for the presence or absence of a phenotypic trait and for a set of polymorphic markers. The distribution of polymorphic markers in an informative meiosis is then analyzed to determine which polymorphic markers co-segregate with a phenotypic trait (Kerem, B. et al., 1989. Science, 245:1073-1080; Yamaoka, L. et al., 1990. Neurology, 40:222-226; Rossiter, B. and Caskey, C, 1991. FASEB J., 5:21-27).

Linkage is analyzed by calculation of lod (log of the odds) values. A lod value is the relative likelihood of obtaining observed segregation data for a marker and a genetic locus when the two are located at a recombination fraction θ, versus the situation in which the two are not linked, and thus segregating independently. A series of likelihood ratios are calculated at various recombination fractions (θ), ranging from θ=0.0 (coincident loci) to θ=0.50 (unlinked). Thus, the likelihood at a given value of θ is the probability of data if loci linked at θ to probability of data if loci unlinked. The computed likelihoods are usually expressed as the log₁₀ of this ratio (i.e., a lod score). For example, a lod score of 3 indicates 1000:1 odds against an apparent observed linkage being a coincidence. The use of logarithms allows data collected from different families to be combined by simple addition. Computer programs are available for the calculation of lod scores for differing values of θ (e.g., LIPED, MLINK; Lathrop, G. et al., 1984. Proc. Nat. Acad. Sci. USA, 81:3443-3446). For any particular lod score, a recombination fraction can be determined from mathematical tables. The value of θ at which the lod score is the highest is considered to be the best estimate of the recombination fraction.

Positive lod score values suggest that the two loci are linked, whereas negative values suggest that linkage is less likely (at that value of θ) than the possibility that the two loci are unlinked. By convention, a combined lod score of +3 or greater (equivalent to greater than 1000:1 odds in favor of linkage) is considered definitive evidence that two loci are linked. Similarly, by convention, a negative lod score of −2 or less is taken as definitive evidence against linkage of the two loci being compared. Negative linkage data are useful in excluding a chromosome or a segment thereof from consideration. The search focuses on the remaining non-excluded chromosomal locations.

Nucleic Acids and Polypeptides of the Invention

All nucleotide positions are relative to SEQ ID NO:1 or GenBank number AL035668, as indicated. The nucleic acids, polypeptides and antibodies described herein can be used in methods of diagnosis of a susceptibility to osteoporosis, as well as in kits useful for diagnosis of a susceptibility to osteoporosis. The reference amino acid sequence for BMP2 is described by SEQ ID NO:2.

An “isolated” nucleic acid molecule, as used herein, is one that is separated from nucleic acids that normally flank the gene or nucleotide sequence (as in genomic sequences) and/or has been completely or partially purified from other transcribed sequences (e.g., as in an RNA library). For example, an isolated nucleic acid of the invention can be substantially isolated with respect to the complex cellular milieu in which it naturally occurs, or culture medium when produced by recombinant techniques, or chemical precursors or other chemicals when chemically synthesized. In some instances, the isolated material will form part of a composition (for example, a crude extract containing other substances), buffer system or reagent mix. In other circumstances, the material can be purified to essential homogeneity, for example as determined by polyacrylamide gel electrophoresis (PAGE) or column chromatography such as HPLC. An isolated nucleic acid molecule of the invention can comprise at least about 50, 80 or 90% (on a molar basis) of all macromolecular species present. With regard to genomic DNA, the term “isolated” also can refer to nucleic acid molecules that are separated from the chromosome with which the genomic DNA is naturally associated. For example, the isolated nucleic acid molecule can contain less than about 5 kb, 4 kb, 3 kb, 2 kb, 1 kb, 0.5 kb or 0.1 kb of the nucleotides that flank the nucleic acid molecule in the genomic DNA of the cell from which the nucleic acid molecule is derived.

The nucleic acid molecule can be fused to other coding or regulatory sequences and still be considered isolated. Thus, recombinant DNA contained in a vector is included in the definition of “isolated” as used herein. Also, isolated nucleic acid molecules include recombinant DNA molecules in heterologous host cells or heterologous organisms, as well as partially or substantially purified DNA molecules in solution. “Isolated” nucleic acid molecules also encompass in vivo and in vitro RNA transcripts of the DNA molecules of the present invention. An isolated nucleic acid molecule or nucleotide sequence can include a nucleic acid molecule or nucleotide sequence that is synthesized chemically or by recombinant means. Therefore, recombinant DNA contained in a vector are included in the definition of “isolated” as used herein. Such isolated nucleotide sequences are useful, for example, in the manufacture of the encoded polypeptide, as probes for isolating homologous sequences (e.g., from other mammalian species), for gene mapping (e.g., by in situ hybridization with chromosomes), or for detecting expression of the gene in tissue (e.g., human tissue), such as by Northern blot analysis or other hybridization techniques.

The invention also pertains to nucleic acid molecules that hybridize under high stringency hybridization conditions, such as for selective hybridization, to a nucleotide sequence described herein (e.g., nucleic acid molecules that specifically hybridize to a nucleotide sequence containing a polymorphic site associated with a haplotype described herein). In one embodiment, the invention includes variants described herein that hybridize under high stringency hybridization and wash conditions (e.g., for selective hybridization) to a nucleotide sequence comprising a nucleotide sequence selected from SEQ ID NO:1 comprising at least one allele at a polymorphic site contained in at least one of the haplotypes described herein polymorphism, or the complement thereof, or a nucleotide sequence encoding an amino acid sequence of SEQ ID NO:2 comprising an altered composition or expression level as the result of an allele contained in a haplotype described herein.

Such nucleic acid molecules can be detected and/or isolated by allele- or sequence-specific hybridization (e.g., under high stringency conditions). “Specific hybridization,” as used herein, refers to the ability of a first nucleic acid to hybridize to a second nucleic acid in a manner such that the first nucleic acid does not hybridize to any nucleic acid other than to the second nucleic acid (e.g., when the first nucleic acid has a higher complementarity to the second nucleic acid than to any other nucleic acid in a sample wherein the hybridization is to be performed). “Stringency conditions” for hybridization is a term of art that refers to the incubation and wash conditions, e.g., conditions of temperature and buffer concentration, that permit hybridization of a particular nucleic acid to a second nucleic acid; the first nucleic acid can be perfectly (i.e., 100%) complementary to the second, or the first and second can share some degree of complementarity that is less than perfect (e.g., 70%, 75%, 85%, 95%). For example, certain high stringency conditions can be used to distinguish perfectly complementary nucleic acids from those of less complementarity. “High stringency conditions”, “moderate stringency conditions” and “low stringency conditions” for nucleic acid hybridizations are explained on pages 2.10.1-2.10.16 and pages 6.3.1-6.3.6 in Current Protocols in Molecular Biology (Ausubel, F. et al., “Current Protocols in Molecular Biology”, John Wiley & Sons, (1998), the entire teachings of which are incorporated by reference herein). The exact conditions that determine the stringency of hybridization depend not only on ionic strength (e.g., 0.2×SSC, 0.1×SSC), temperature (e.g., room temperature, 42° C., 68° C.) and the concentration of destabilizing agents such as formamide or denaturing agents such as SDS, but also on factors such as the length of the nucleic acid sequence, base composition, percent mismatch between hybridizing sequences and the frequency of occurrence of subsets of that sequence within other non-identical sequences. Thus, equivalent conditions can be determined by varying one or more of these parameters while maintaining a similar degree of identity or similarity between the two nucleic acid molecules. Typically, conditions are used such that sequences at least about 60%, at least about 70%, at least about 80%, at least about 90% or at least about 95% or more identical to each other remain hybridized to one another. By varying hybridization conditions from a level of stringency at which no hybridization occurs to a level at which hybridization is first observed, conditions that will allow a given sequence to hybridize (e.g., selectively) with the most complementary sequences in the sample can be determined.

Exemplary conditions that describe the determination of wash conditions for moderate or low stringency conditions are described in Kraus, M. and Aaronson, S., Methods Enzymol., 200:546-556 (1991); and in, Ausubel, F. et al., “Current Protocols in Molecular Biology”, John Wiley & Sons, (1998). Washing is the step in which conditions are usually set so as to determine a minimum level of complementarity of the hybrids. Generally, starting from the lowest temperature at which only homologous hybridization occurs, each ° C., by which the final wash temperature is reduced (holding SSC concentration constant) allows an increase by 1% in the maximum mismatch percentage among the sequences that hybridize. Generally, doubling the concentration of SSC results in an increase in Tₘ of about 17° C. Using these guidelines, the wash temperature can be determined empirically for high, moderate or low stringency, depending on the level of mismatch sought.

For example, a low stringency wash can comprise washing in a solution containing 0.2×SSC/0.1% SDS for 10 minutes at room temperature; a moderate stringency wash can comprise washing in a pre-warmed solution (42° C.) solution containing 0.2×SSC/0.1% SDS for 15 minutes at 42° C.; and a high stringency wash can comprise washing in pre-warmed (68° C.) solution containing 0.1×SSC/0.1% SDS for 15 minutes at 68° C. Furthermore, washes can be performed repeatedly or sequentially to obtain a desired result as known in the art. Equivalent conditions can be determined by varying one or more of the parameters given as an example, as known in the art, while maintaining a similar degree of complementarity between the target nucleic acid molecule and the primer or probe used (e.g., the sequence to be hybridized).

The percent identity of two nucleotide or amino acid sequences can be determined by aligning the sequences for optimal comparison purposes (e.g., gaps can be introduced in the sequence of a first sequence). The nucleotides or amino acids at corresponding positions are then compared, and the percent identity between the two sequences is a function of the number of identical positions shared by the sequences (i.e., % identity=# of identical positions/total # of positions×100). In certain embodiments, the length of a sequence aligned for comparison purposes is at least 30%, at least 40%, at least 60%, at least 70%, at least 80% or at least 90% of the length of the reference sequence. The actual comparison of the two sequences can be accomplished by well-known methods, for example, using a mathematical algorithm. A non-limiting example of such a mathematical algorithm is described in Karlin, S. and Altschul, S., Proc. Natl. Acad. Sci. USA, 90:5873-5877 (1993). Such an algorithm is incorporated into the NBLAST and XBLAST programs (version 2.0) as described in Altschul, S. et al., Nucleic Acids Res., 25:3389-3402 (1997). When utilizing BLAST and Gapped BLAST programs, the default parameters of the respective programs (e.g., NBLAST) can be used. See the website on the world wide web at ncbi.nlm.nih.gov. In one embodiment, parameters for sequence comparison can be set at score=100, wordlength=12, or can be varied (e.g., W=5 or W=20).

Another non-limiting example of a mathematical algorithm utilized for the comparison of sequences is the algorithm of Myers and Miller, CABIOS (1989). Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis, A. and Robotti, C., 1994. Comput. Appl. Biosci., 10:3-5; and FASTA described in Pearson, W. and Lipman, D., 1988. Proc. Natl. Acad. Sci. USA, 85:2444-8.

In another embodiment, the percent identity between two amino acid sequences can be accomplished using the GAP program in the GCG software package (Accelrys, Cambridge, UK) using either a Blossom 63 matrix or a PAM250 matrix, and a gap weight of 12, 10, 8, 6, or 4 and a length weight of 2, 3, or 4. In yet another embodiment, the percent identity between two nucleic acid sequences can be accomplished using the GAP program in the GCG software package, using a gap weight of 50 and a length weight of 3.

The present invention also provides isolated nucleic acid molecules that contain a fragment or portion that hybridizes under highly stringent conditions to a nucleotide sequence comprising a nucleotide sequence selected from SEQ ID NO:1 and comprising at least one allele contained in one or more haplotypes described herein, and the complement thereof. The invention also provides isolated nucleic acid molecules that contain a fragment or portion that hybridizes under highly stringent conditions to a nucleotide sequence encoding an amino acid sequence selected from SEQ ID NO:2, a polymorphic variant thereof, or a fragment or portion thereof. The nucleic acid fragments of the invention are at least about 15, at least about 18, 20, 23 or 25 nucleotides, and can be 30, 40, 50, 100, 200 or more nucleotides in length. Longer fragments, for example, 30 or more nucleotides in length, which encode antigenic polypeptides described herein, are particularly useful, such as for the generation of antibodies as described below.

The nucleic acid fragments of the invention are used as probes or primers in assays such as those described herein. “Probes” or “primers” are oligonucleotides that hybridize in a base-specific manner to a complementary strand of nucleic acid molecules. In addition to DNA and RNA, such probes and primers include polypeptide nucleic acids (PNA), as described in Nielsen, P. et al., 1991. Science, 254:1497-1500.

A probe or primer comprises a region of nucleotide sequence that hybridizes to at least about 15, typically about 20-25, and in certain embodiments about 40, 50 or 75, consecutive nucleotides of a nucleic acid molecule comprising a contiguous nucleotide sequence from SEQ ID NO:1 and comprising at least one allele contained in one or more haplotypes described herein, and the complement thereof. The invention also provides isolated nucleic acid molecules that contain a fragment or portion that hybridizes under highly stringent conditions to a nucleotide sequence encoding an amino acid sequence selected from SEQ ID NO:2, a polymorphic variant thereof, or a fragment or portion thereof. In particular embodiments, a probe or primer can comprise 100 or fewer nucleotides; for example, in certain embodiments from 6 to 50 nucleotides, or for example from 12 to 30 nucleotides. In other embodiments, the probe or primer is at least 70% identical to the contiguous nucleotide sequence or to the complement of the contiguous nucleotide sequence, for example at least 80% identical in certain embodiments, at least 85% identical in other embodiments, at least 90% identical, and in other embodiments at least 95% identical, or even capable of selectively hybridizing to the contiguous nucleotide sequence or to the complement of the contiguous nucleotide sequence. Often, the probe or primer further comprises a label, e.g., radioisotope, fluorescent compound, enzyme, or enzyme co-factor.

The nucleic acid molecules of the invention such as those described above can be identified and isolated using standard molecular biology techniques and the sequence information provided in SEQ ID NO:1. For example, nucleic acid molecules can be amplified and isolated by the polymerase chain reaction using synthetic oligonucleotide primers designed based on one or more of the sequences provided in SEQ ID NO:1 (and optionally comprising at least one allele contained in one or more haplotypes described herein) and/or the complement thereof. See generally PCR Technology: Principles and Applications for DNA Amplification (ed. H. A. Erlich, Freeman Press, NY, N.Y., 1992); PCR Protocols: A Guide to Methods and Applications (Eds. Innis, et al., Academic Press, San Diego, Calif., 1990); Mattila, P. et al., 1991. Nucleic Acids Res., 19:4967-4973; Eckert, K. and Kunkel, T., 1991. PCR Methods and Applications, 1:17-24; PCR (eds. McPherson et al., IRL Press, Oxford); and U.S. Pat. No. 4,683,202. The nucleic acid molecules can be amplified using cDNA, mRNA or genomic DNA as a template, cloned into an appropriate vector and characterized by DNA sequence analysis.

Other suitable amplification methods include the ligase chain reaction (LCR; see Wu, D. and Wallace, R., 1989. Genomics, 4:560-469; Landegren, U. et al., 1988. Science, 241:1077-1080), transcription amplification (Kwoh, D. et al., 1989. Proc. Natl. Acad. Sci. USA, 86:1173-1177), and self-sustained sequence replication (Guatelli, J. et al., 1990. Proc. Nat. Acad Sci. USA, 87:1874-1878) and nucleic acid based sequence amplification (NASBA). The latter two amplification methods involve isothermal reactions based on isothermal transcription, which produce both single-stranded RNA (ssRNA) and double-stranded DNA (dsDNA) as the amplification products in a ratio of about 30 and 100 to 1, respectively.

The amplified DNA can be labeled, for example radiolabeled, and used as a probe for screening a cDNA library derived from human cells. The cDNA can be derived from mRNA and contained in zap express (Stratagene, La Jolla, Calif.), ZIPLOX (Gibco BRL, Gaithesburg, Md.) or other suitable vector. Corresponding clones can be isolated, DNA can obtained following in vivo excision, and the cloned insert can be sequenced in either or both orientations by art recognized methods to identify the correct reading frame encoding a polypeptide of the appropriate molecular weight. For example, the direct analysis of the nucleotide sequence of nucleic acid molecules of the present invention can be accomplished using well known methods that are commercially available. See, for example, Sambrook et al., Molecular Cloning, A Laboratory Manual (2nd Ed., CSHP, New York 1989); Zyskind et al., Recombinant DNA Laboratory Manual, (Acad. Press, 1988)). Additionally, fluorescence methods are also available for analyzing nucleic acids (Chen, X. et al., 1999. Genome Res., 9:492-498) and polypeptides. Using these or similar methods, the polypeptide and the DNA encoding the polypeptide can be isolated, sequenced and further characterized.

In general, the isolated nucleic acid sequences of the invention can be used as molecular weight markers on Southern gels, and as chromosome markers that are labeled to map related gene positions. The nucleic acid sequences can also be used to compare with endogenous DNA sequences in patients to identify genetic disorders (e.g., a predisposition for or susceptibility to osteoporosis), and as probes, such as to hybridize and discover related DNA sequences or to subtract out known sequences from a sample. The nucleic acid sequences can further be used to derive primers for genetic fingerprinting, to raise anti-polypeptide antibodies using immunization techniques, and as an antigen to raise anti-DNA antibodies or elicit immune responses.

As used herein, two polypeptides (or a region of the polypeptides) are substantially homologous or identical when the amino acid sequences are at least about 45-55%, in certain embodiments at least about 70-75%, in other embodiments at least about 80-85%, and in other embodiments greater than about 90% or more homologous or identical. A substantially homologous amino acid sequence, according to the present invention, will be encoded by a nucleic acid molecule hybridizing to SEQ ID NO:1 and optionally comprising at least one allele contained in the haplotypes described herein, under stringent conditions as more particularly described above or will be encoded by a nucleic acid molecule hybridizing to a nucleic acid sequence encoding SEQ ID NO:2 portion thereof or polymorphic variant thereof, under stringent conditions as more particularly described thereof.

A variant polypeptide can differ in amino acid sequence by one or more substitutions, deletions, insertions, inversions, fusions, and truncations or a combination of any of these. Further, variant polypeptides can be fully functional or can lack function in one or more activities. Fully functional variants typically contain only conservative variation or variation in non-critical residues or in non-critical regions. Functional variants can also contain substitution of similar amino acids that result in no change or an insignificant change in function. Alternatively, such substitutions can positively or negatively affect function to some degree. Non-functional variants typically contain one or more non-conservative amino acid substitutions, deletions, insertions, inversions, or truncation or a substitution, insertion, inversion, or deletion in a critical residue or critical region.

Amino acids that are essential for function can be identified by methods known in the art, such as site-directed mutagenesis or alanine-scanning mutagenesis (Cunningham, B and Wells, J., 1989. Science, 244:1081-1085). The latter procedure introduces single alanine mutations at every residue in the molecule. The resulting mutant molecules are then tested for biological activity in vitro. Sites that are critical for polypeptide activity can also be determined by structural analysis, for example, by crystallization, nuclear magnetic resonance or photoaffinity labeling (Smith, L. et al., 1992. J. Mol. Biol., 224:899-904; de Vos, A. et al., 1992. Science, 255:306-312).

The isolated polypeptide can be purified from cells that naturally express it, purified from cells that have been altered to express it (recombinant), or synthesized using known protein synthesis methods. In one embodiment, the polypeptide is produced by recombinant DNA techniques. For example, a nucleic acid molecule encoding the polypeptide is cloned into an expression vector, the expression vector introduced into a host cell and the polypeptide expressed in the host cell. The polypeptide can then be isolated from the cells by an appropriate purification scheme using standard protein purification techniques.

In general, polypeptides of the present invention can be used as a molecular weight marker on SDS-PAGE gels or on molecular sieve gel filtration columns using art-recognized methods. The polypeptides of the present invention can be used to raise antibodies or to elicit an immune response. The polypeptides can also be used as a reagent, e.g., a labeled reagent, in assays to quantitatively determine levels of the polypeptide or a molecule to which it binds (e.g., a receptor or a ligand) in biological fluids. The polypeptides can also be used as markers for cells or tissues in which the corresponding polypeptide is preferentially expressed, either constitutively, during tissue differentiation, or in a diseased state. The polypeptides can be used to isolate a corresponding binding partner, e.g., receptor or ligand, such as, for example, in an interaction trap assay, and to screen for peptide or small molecule antagonists or agonists of the binding interaction.

Antibodies of the Invention

Polyclonal and/or monoclonal antibodies that specifically bind one form of the gene product but not to the other form of the gene product are also provided. Antibodies are also provided that bind a portion of either the variant or the reference gene product that contains the polymorphic site or sites. The invention provides antibodies to polypeptides having an amino acid sequence of SEQ ID NO:2 or a variant BMP2 polypeptide. The term “antibody” as used herein refers to immunoglobulin molecules and immunologically active portions of immunoglobulin molecules, i.e., molecules that contain an antigen binding site that specifically binds an antigen. A molecule that specifically binds to a polypeptide of the invention is a molecule that binds to that polypeptide or a fragment thereof, but does not substantially bind other molecules in a sample, e.g., a biological sample that naturally contains the polypeptide. Examples of immunologically active portions of immunoglobulin molecules include F(ab) and F(ab′)₂ fragments that can be generated by treating the antibody with an enzyme such as pepsin. The invention provides polyclonal and monoclonal antibodies that bind to a polypeptide of the invention. The term “monoclonal antibody” or “monoclonal antibody composition”, as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

Polyclonal antibodies can be prepared as described above by immunizing a suitable subject with a desired immunogen, e.g., polypeptide of the invention or fragment thereof. The antibody titer in the immunized subject can be monitored over time by standard techniques, such as with an enzyme linked immunosorbent assay (ELISA) using an immobilized polypeptide. If desired, the antibody molecules directed against the polypeptide can be isolated from the mammal (e.g., from the blood) and further purified by well-known techniques, such as protein A chromatography, to obtain the IgG fraction. At an appropriate time after immunization, e.g., when the antibody titers are highest, antibody-producing cells can be obtained from the subject and used to prepare monoclonal antibodies by standard techniques, such as the hybridoma technique (Kohler, G. and Milstein, C., 1975. Nature, 256:495-497), the human B cell hybridoma technique (Kozbor, D. et al., 1983. Immunol. Today, 4:72), the EBV-hybridoma technique (Cole et al., 1985. Monoclonal Antibodies and Cancer Therapy, Alan R. Liss, Inc., pp. 77-96) or trioma techniques.

The technology for producing hybridomas is well known (see generally Current Protocols in Immunology (1994) Coligan et al. (eds.) John Wiley & Sons, Inc., New York, N.Y.). Briefly, an immortal cell (typically a myeloma) is fused to a lymphocyte (typically a splenocyte) from a mammal immunized with an immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cells can be applied for the purpose of generating a monoclonal antibody to a polypeptide of the invention (see, e.g., Current Protocols in Immunology, supra; Galfre, G. et al., 1977. Nature, 266:550-552; Kenneth, R., in Monoclonal Antibodies A New Dimension In Biological Analyses, Plenum Publishing Corp., New York, N.Y. (1980); and Lerner, E., 1981. Yale J. Biol. Med., 54:387-402). Moreover, the ordinarily skilled worker will appreciate that there are many variations of such methods that also would be useful.

Alternative to preparing monoclonal antibody-secreting hybridomas, a monoclonal antibody to a polypeptide of the invention can be identified and isolated by screening a recombinant combinatorial immunoglobulin library (e.g., an antibody phage display library) with the polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs, P. et al., 1991. Biotechnology (NY), 9:1369-1372; Hay, B. et al., 1992. Hum. Antibodies Hybridomas, 3:81-85; Huse, W. et al., 1989. Science, 246:1275-1281; Griffiths, A. et al., 1993. EMBO J., 12:725-734.

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

In general, antibodies of the invention (e.g., a monoclonal antibody) can be used to detect a polypeptide (e.g., in a cellular lysate, cell supernatant, or tissue sample) in order to evaluate the abundance and pattern of expression of the polypeptide. Antibodies can be used diagnostically to monitor protein levels in tissue as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S, ³²P, ³³P, ¹⁴C or ³H.

Statistical Analysis

For single marker association to the disease, the Fisher exact test can be used to calculate two-sided p-values for each individual allele. All p-values are presented unadjusted for multiple comparisons unless specifically indicated. The presented frequencies (for microsatellites, SNPs and haplotypes) are allelic frequencies as opposed to carrier frequencies. To minimize any bias due the relatedness of the patients who were recruited as families for the linkage analysis, first and second-degree relatives can be eliminated from the patient list. Furthermore, the test can be repeated for association correcting for any remaining relatedness among the patients, by extending a variance adjustment procedure (Risch, N. and Teng, J., 1998. Genome Res., 8:1273-1288), DNA pooling for sibships so that it can be applied to general familial relationships, and present both adjusted and unadjusted p-values for comparison. The differences are in general very small as expected. To assess the significance of single-marker association corrected for multiple testing we carried out a randomization test using the same genotype data. Cohorts of patients and controls can be randomized and the association analysis redone multiple times (e.g., up to 500,000 times) and the p-value is the fraction of replications that produced a p-value for some marker allele that is lower than or equal to the p-value we observed using the original patient and control cohorts.

For both single-marker and haplotype analyses, relative risk (RR) and the population attributable risk (PAR) can be calculated assuming a multiplicative model (haplotype relative risk model; Terwilliger, J. and Ott, J., 1992. Hum. Hered., 42:337-46; Falk, C. and Rubinstein, P., 1987. Ann. Hum. Genet., 51 (Pt 3):227-33), i.e., that the risks of the two alleles/haplotypes a person carries multiply. For example, if RR is the risk of A relative to a, then the risk of a person homozygote AA will be RR times that of a heterozygote Aa and RR² times that of a homozygote aa. The multiplicative model has a nice property that simplifies analysis and computations-haplotypes are independent, i.e., in Hardy-Weinberg equilibrium, within the affected population as well as within the control population. As a consequence, haplotype counts of the affecteds and controls each have multinomial distributions, but with different haplotype frequencies under the alternative hypothesis. Specifically, for two haplotypes, hᵢ and hⱼ, risk(hᵢ)/risk(hⱼ)=(fᵢ/pᵢ)/(fⱼ/pⱼ), where f and p denote, respectively, frequencies in the affected population and in the control population. While there is some power loss if the true model is not multiplicative, the loss tends to be mild except for extreme cases. Most importantly, p-values are always valid since they are computed with respect to null hypothesis.

In general, haplotype frequencies are estimated by maximum likelihood and tests of differences between cases and controls are performed using a generalized likelihood ratio test. The haplotype analysis program, NEMO, which stands for NEsted MOdels, can be used to calculate all of the haplotype results. To handle uncertainties with phase and missing genotypes, it is emphasized that we do not use a common two-step approach to association tests, where haplotype counts are first estimated, possibly with the use of the EM algorithm, (Dempster, A. P., Laird, N. M. & Rubin, D. B., J. R. Stat. Soc. B 39:1-38 (1977)) and then tests are performed treating the estimated counts as though they are true counts, a method that can sometimes be problematic and may require randomization to properly evaluate statistical significance. Instead, with NEMO, maximum likelihood estimates, likelihood ratios and p-values are computed with the aid of the EM-algorithm directly for the observed data, and hence the loss of information due to uncertainty with phase and missing genotypes is automatically captured by the likelihood ratios. Even so, it is of interest to know how much information is retained, or lost, due to incomplete information. Described herein is such a measure that is natural under the likelihood framework. For a fixed set of markers, the simplest tests performed compare one selected haplotype against all of the others. Call the selected haplotype h1 and the others h₂, . . . , hₖ. Let p₁, . . . , pₖ denote the population frequencies of the haplotypes in the controls, and f₁, . . . , fₖ denote the population frequencies of the haplotypes in the affecteds. Under the null hypothesis, fᵢ=pᵢ for all i. The alternative model that we use for the test assumes h₂, . . . , hₖ to have the same risk while h₁ is allowed to have a different risk. This implies that while p₁ can be different from f₁, fᵢ/(f₂+ . . . +fₖ)=pᵢ/(p₂+ . . . +pₖ)=βᵢ for i=2, . . . , k. Denoting f₁/p₁ by r, and noting that β₂+ . . . +βₖ=1, the test statistic based on generalized likelihood ratios is
Λ=2[l(\hat{r}, \hat{p}₁, \hat{β}₂, . . . , \hat{β}ₖ₋₁)−l(1, \tilde{p}₁, \tilde{β}₂, . . . , \tilde{β}ₖ₋₁)
where l denotes loge likelihood and ~ and ˆ denote maximum likelihood estimates under the null hypothesis and alternative hypothesis, respectively. A has asymptotically a chi-square distribution with 1-df, under the null hypothesis. Slightly more complicated null and alternative hypotheses can also be used. For example, let h, be G0, h₂be GX and h₃ be AX. When comparing G0 against GX, i.e., this is the test which gives estimated RR of 1.46 and p-value=0.0002, the null assumes G0 and GX have the same risk but AX is allowed to have a different risk. The alternative hypothesis allows, for example, three haplotype groups to have different risks. This implies that, under the null hypothesis, there is a constraint that f₁/p₁=f₂/p₂, or w=[f₁/p₁]/[f₂/p₂]=1. The test statistic based on generalized likelihood ratios is
Λ=2[l(\hat{p}₁, \hat{f}₁, \hat{p}₂, \hat{f}₂, ŵ)−l(\tilde{p}₁, \tilde{f}₁, \tilde{p}₂, 1)
that again has asymptotically a chi-square distribution with 1-df under the null hypothesis. If there are composite haplotypes (for example, h₂ and h₃), that is handled in a natural manner under the nested models framework.
Linkage Disequilibrium Using NEMO

LD between pairs of SNPs can be calculated using the standard definition of D′ and R² (Lewontin, R., 1964. Genetics, 49:49-67); Hill, W. and Robertson, A., 1968. Theor. Appl. Genet., 22:226-231). Using NEMO, frequencies of the two marker allele combinations are estimated by maximum likelihood and deviation from linkage equilibrium is evaluated by a likelihood ratio test. The definitions of D′ and R² are extended to include microsatellites by averaging over the values for all possible allele combination of the two markers weighted by the marginal allele probabilities.

Statistical Methods for Linkage Analysis

Multipoint, affected-only allele-sharing methods can be used in the analyses to assess evidence for linkage. Results, both the LOD-score and the non-parametric linkage (NPL) score, can be obtained using the program Allegro (Gudbjartsson, D. et al., 2000. Nat. Genet., 25:12-3). The baseline linkage analysis uses the Sₚₐᵢᵣₛ scoring function (Whittemore, A. and Halpern, J., 1994. Biometrics, 50:118-27; Kruglyak L. et al., 1996. Am. J. Hum. Genet., 58:1347-63), the exponential allele-sharing model (Kong, A. and Cox, N., 1997. Am. J. Hum. Genet., 61:1179-88) and a family weighting scheme that is halfway, on the log-scale, between weighting each affected pair equally and weighting each family equally. The information measure used is part of the Allegro program output and the information value equals zero if the marker genotypes are completely uninformative and equals one if the genotypes determine the exact amount of allele sharing by decent among the affected relatives (Gretarsdottir, S. et al., 2002. Am. J. Hum. Genet., 70:593-603).

The invention will be further described by the following non-limiting example. The teachings of all publications cited herein are incorporated herein by reference in their entirety.

### EXEMPLIFICATION

### Example 1

### Identification of BMP2 Haplotypes.

Haplotypes spanning the BMP2 nucleic acid sequence that are associated to osteoporosis have been identified.

“Haplotype I”, “Haplotype II”, “Haplotype a”, “Haplotype b”, “Haplotype c” and “Haplotype d” are described below in Table 1; hapG and hapV are shown in FIGS. 14A and 14B. Each haplotype comprises alleles at more than one polymorphic site (haplotype I comprises 4 SNPs and a microsatellite; haplotype II comprises 3 SNPs and 2 microsatellites; haplotype a comprises 2 SNPs; haplotype b comprises 3 SNPs; haplotype c comprises 3 SNPs; and haplotype d comprises 3 SNPs).

The actual haplotypes involve the markers listed in Table 1 and in FIGS. 14A and 14B.

TABLE 1
Haplotypes linked to osteoporosis.

| | | | | | haplo- |
|---|---|---|---|---|---|
| haplo- | | | | pos. | type |
| type | marker | type | allele # | AL035668 | allele |
| hapI | TSC0898956 | SNP | 1 | 114671 | C |
| hapI | B420 | SNP | 0 | 118920 | A |
| hapI | B8463 | SNP | 3 | 126963 | T |
| hapI | D20S846 | microsat- | 6 | 135601- | |
| | | ellite | | 136526 | |
| hapI | TSC0191642 | SNP | 3 | 139007 | T |
| hapII | P4337 | SNP | 3 | 112887 | T |
| hapII | D20S892 | microsat- | 10 | 121625- | |
| | | ellite | | 121661 | |
| hapII | B5048 | SNP | 1 | 123548 | C |
| hapII | B9082 | SNP | 2 | 127582 | G |
| hapII | D20S59 | microsat- | 6 | 162787- | |
| | | ellite | | 162827 | |
| hap-a | B7111/ | SNP | 2 | 125611 | G |
| | rs235764 | | | | |
| hap-a | B12845/ | SNP | 1 | 131345 | C |
| | rs15705 | | | | |
| hap-b | P9313 | SNP | 3 | 117863 | T |
| hap-b | B10631 | SNP | 2 | 129131 | G |
| hap-b | D35548 | SNP | 3 | 167584 | T |
| hap-c | rs1116867 | SNP | 0 | 149529 | A |
| hap-c | TSC0278787 | SNP | 0 | 154077 | A |
| hap-c | D35548 | SNP | 3 | 167584 | T |
| hap-d | TSC0271643/ | SNP | 3 | upstream | T |
| | rs965291 | | | | |
| hap-d | P9313 | SNP | 3 | 117863 | T |
| hap-d | B7111 | SNP | 2 | 125611 | G |

Alleles #'s: For SNP alleles A = 0, C = 1, G = 2, T = 3;
for microsatellite alleles: the CEPH sample 1347-02 (CEPH genomics repository) is used as a reference, the lower allele of each microsatellite in this sample is set at 0 and all other alleles in other samples are numbered according in relation to this reference.
Thus allele1 is 1 bp longer than the lower allele in the CEPH sample 1347-02, allele 2 is 2 bp longer than the lower allele in the CEPH sample 1347-02, allele 3 is 3 bp longer than the lower allele in the CEPH sample 1347-02, allele 4 is 4 bp longer than the lower allele in the CEPH sample 1347-02, allele-1 is 1 bp shorter than the lower allele in the CEPH sample 1347-02, allele-2 is 2 bp shorter than the lower allele in the CEPH sample 1347-02, and so on.

Haplotype Analysis

Haplotypes were identified as described above and haplotype analysis was performed as described elsewhere (Stefansson, H. et al., 2002. Am. J. Hum. Genet., 71:877-92).

Phenotypes and Control Samples for Osteoporosis

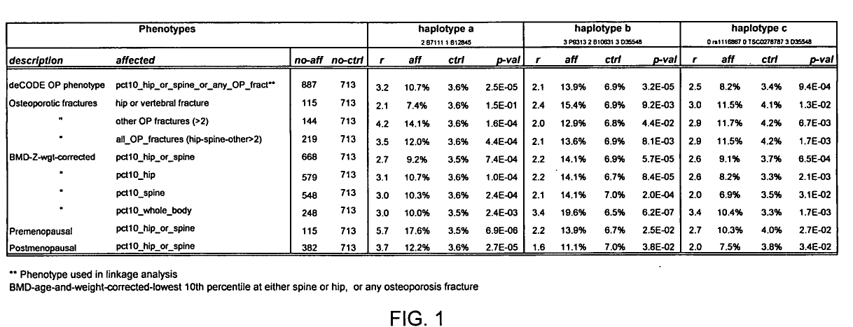

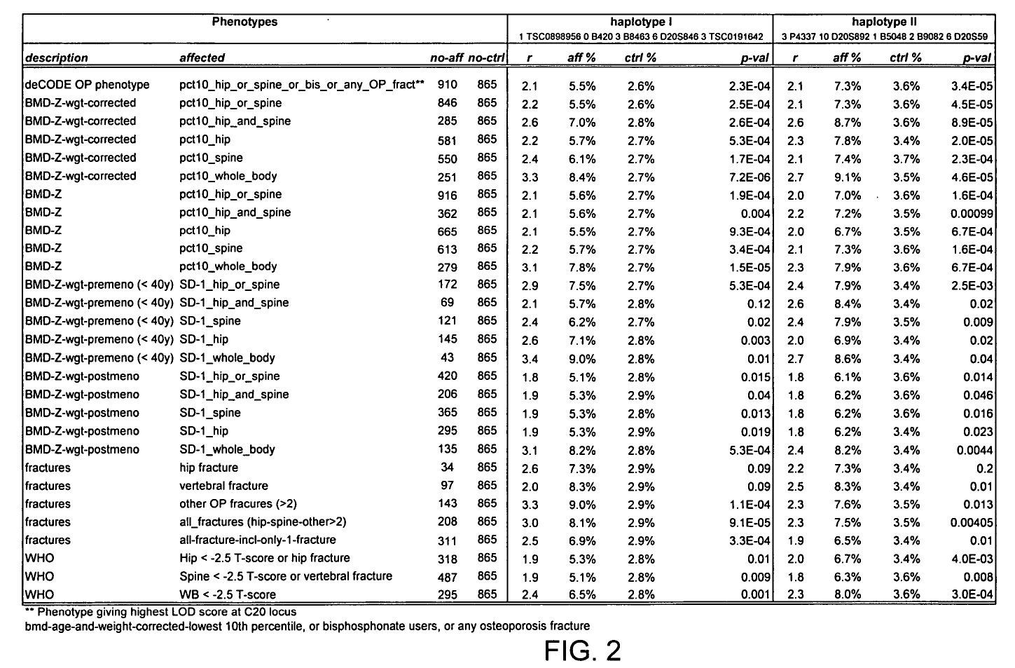

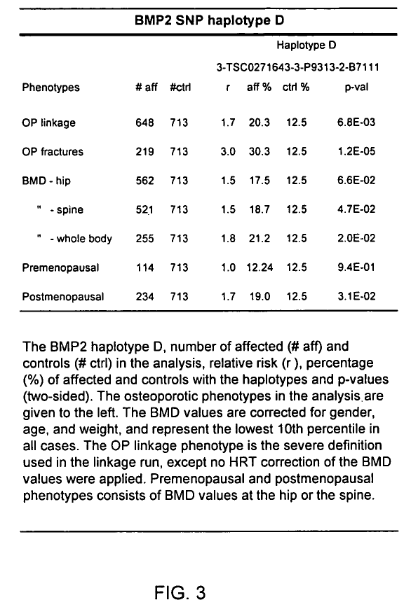

Several different osteoporotic phenotypes were used in the haplotype analysis; including phenotypes used in linkage analysis as well as other osteoporosis-related phenotypes. The relationship between various phenotypes and haplotypes a, b and c are shown in FIG. 1 and FIG. 3. Haplotypes I and II are shown in FIG. 2.

For association analysis, the material collected for the linkage analysis was used, as well as all sporadic individuals with a Z-score less than −1 SD. The control group comprised two randomly collected groups from the general population; one with BMD measurements and questionnaire information, the other with no medical information. These groups served as randomly collected population based controls, unrelated within 5 meiotic events; the total number of members in both groups was 1272.

The BMD of all participants, patients as well as relatives, was determined using dual energy X-ray absorptiometry at the lumbar spine (L2-L4) in posterior-anterior projection, and total hip (proximal end of femur) and whole body (QDR 4500A, Hologic, Waltham, Mass.). Weight and height were measured at the time of BMD measurement. All participants completed a detailed questionnaire regarding their medical history, menstrual periods, current and past medications (including hormone replacement therapy (HRT)), and history of all fractures and trauma.

### Example 2

### Identification of the BMP2 Nucleic Acid With Linkage to Osteoporosis

Phenotype and Family Construction

Patients who have low impact fractures and/or take bisphosphonates for treating osteoporosis are automatically treated as affecteds. People with low bone mass density (BMD) measurements are considered to be osteoporotic, and have been shown to have substantially increased risk of fractures. BMD measurements are taken for both the hip and the spine. For each person with BMD measurements, a standardized BMD score is computed (mean 0, standard deviation 1 for the population), which is adjusted for sex, age, body weight and hormone replacement therapy (HRT). For the combined analysis, the two measurements are summed. Population BMD data from Iceland and the United States are used for standardization and adjustment. For example, a person with a positive BMD score is above average and one with a negative score is below average for his/her age, body weight and possibly HRT. Assuming approximate normality, a score of −1 corresponds approximately to the lower 16^{th} percentile, etc.

For analysis, we start with a current list of primary people, people who have BMD measurements and/or are severely affected, and for whom we have genotypes. We then use the genealogy database to create family clusters linking these primary people using a threshold distance of 5 meiotic events. This procedure produced 190 potentially informative clusters with a total of 1215 primary people.

Linkage Data

Four genome wide scans (GWS) were performed using osteoporotic phenotypes at different skeletal sites; the hip, the spine, and combined phenotypes. All GWS analysis located at 20 cM region on Chr20, between 10 cM and 30 cM based on the Marshfield map.

All of the analyses were performed using the Allegro linkage program developed at deCODE (Gudbjartsson et al., Nature Genetics, 25: 12-13, May 2000). The allele sharing analysis uses the Sₚₐᵢᵣₛ scoring function of GENEHUNTER (Kruglyak et al., Am. J. Hum. Genet., 46: 1347-1363, 1996), but families were weighted using a scheme that is a compromise between weighting families equally and weighting affected pairs equally. The allele-sharing LOD scores were computed using the ‘exponential model’ described in Kong and Cox, Am. J. Hum. Genet., 61: 1179-1188 (1997).

Hip

The phenotype used was age, sex, weight and HRT corrected BMD<−1 SD at the hip (total hip). Hip fracture cases and bisphosphonate users are also considered affected even if values are above −1 SD. A total of 346 affected were used in this analysis. The GWS resulted in a LOD score of 3.1 using our standard set of markers. Adding 10 extra markers at the region on interest, between 11 cM and 39 cM, resulted in a LOD score of 3.3.

Spine

The phenotype was age, sex, weight and HRT corrected BMD<−1 SD at lumbar spine (L2-L4). Vertebral compression fracture cases and bisphosponate users are also considered affected even if values are above −1 SD. A total of 402 affected people were used in this analysis. The GWS resulted in a LOD score of 2.4 at the same location as in the hip analysis using the standard set of markers, but a LOD score of 2.9 with the extra marker set.

Combined

The phenotype used was the sum of corrected BMD<−1.5 SD. Vertebral compression fracture, hip fracture, other osteoporosis related low impact fracture (at least two fractures) and bisphosphonate users (BMD measurements before treatment start are used if available) are all considered affected. A total of 522 affected were used in this analysis. The GWS resulted in a LOD score of 2.5 with the standard marker set, but a LOD score of 3.9 using the extra markers in the region.

Combined Severe

The phenotype used was the sum of the age, sex, weight and HRT corrected BMD<−2.3 SD. Vertebral compression fracture, hip fracture, other osteoporosis related low impact fracture (at least two fractures) and bisphosphonate users affected. The number of affected in this analysis was 290. The GWS resulted in a LOD score of 3.8 with the standard set but a LOD score of 4.7 was reached using the extra 10 markers in addition.

Corticosteroid users and women with early menopause were excluded as affected in all analysis.

The BMP2 Gene

The BMP2 nucleic acid is located in this region. Only 5 kb are between the marker D20S846, which gives the highest LOD score, and the 3′ end of the gene. The gene has been sequenced and characterized in terms of exon/intron structures, promoter region and transcriptional start sites. This information are publicly available.

A number of nucleotide changes are observed in the Icelandic population. These changes have not to our knowledge been described before (See Table 2).

BMP2 binds to the receptors BMPR-IA or BMPR-IB, and BMPR-II, leading to formation of receptor complex heterodimer and phosphorylation of the BMPR-IA or BMPR-IB receptors. Once activated, these receptors subsequently phosphorylate SMAD1, SMAD5 or SMAD8, which in turn form complexes with SMAD4 and translocate to the nucleus where the transcription of specific genes is affected (Massague, J., Annu. Rev. Biochem., 67:753-791 (1998); Chen, D. et al., J. Cell Biol., 142(1):295-305 (1998)). SMADs 6 and 7 block signals by preventing the activation of SMAD1, SMAD5 or SMAD8 by the BMP2 receptors and have been shown to inhibit osteoblast differentiation (Miyazono, K., Bone, 25(1):91-93 (1999); Fujii, M., et al., Mol. Biol. Cell, 10(11):3801-3813 (1999)). BMP2 stimulates Cbfal, alkaline phosphatase and Collagen type I (osteoblast specific proteins) expression through BMPR-IB (Chen, D. et al., J. Cell Biol., 142(1):295-305 (1998). Cbfal regulates the expression of osteoprotegerin (OPG), which is an osteoblast-secreted glycoprotein that functions as a potent inhibitor of osteoblast differentiation and thus of bone resorption (Thirunavukkarasu, K., et al., J. Biol. Chem., (2000)). Cbfal controls osteoblast differentiation and bone formation. During cellular aging of human osteoblasts, there is a significant reduction (up to 50%) of Cbfal mRNA (Christiansen, M., et al., J. Gerontol. A Biol. Sci. Med. Sci., 55(4):B 194-200 (2000)).

Results and Discussion

As a result of the linkage studies, the analysis shows that this locus is involved in multiple osteoporosis phenotypes. Furthermore, mutation within the human BMP2 nucleic acid is likely to explain the phenotypes in these families. Sporadic occurrence of osteoporosis, i.e., occurrence without familial connection, can also be determined using the information contained herein.

Osteoporosis could be caused by a defect in the BMP2 nucleic acid as follows: An alteration in the BMP2 nucleic acid (transcription, splice, protein variant etc.) could lead to a reduction of its action on Cbfal through BMPR-IB and the subsequent signaling pathway. This would lead to less bone formation because of fewer and less active osteoblasts and more bone resorption because of less OPG and more osteoclasts. This would lead to bone loss. Since a significant reduction of Cbfal levels is associated with aging osteoblasts, this effect could become more important with older age.

TABLE 2

| LOCUS | 14759 bp DNA |
|---|---|
| DEFINITION | Human bone morphogenetic protein 2 (BMP2) gene, |
| | complete cds, |
| | complete sequence. |
| ACCESSION | |
| VERSION | |
| KEYWORDS | . |
| SOURCE | human. |
| ORGANISM | Homo sapiens |
| | Eukaryota; Metazoa; Chordata; Craniata; Vertebrata; |
| | Mammalia; Eutheria; Primates; Catarrhini; Hominidae; |
| | Homo. |
| REFERENCE | 1 (bases 1-14759) |
| AUTHORS | Blakey, S. |
| TITLE | Direct Submission |
| JOURNAL | Submitted (04-APR-2000) Sanger Centre, Hinxton, |
| | Cambridgeshire, CB10 1SA, UK. E-mail enquiries: |
| | humquery@sanger.ac.uk Clone requests: |
| | clonerequest@sanger.ac.uk |
| COMMENT | This sequence was taken from GenBank sequence |
| | AL035668 (VERSION AL035668.15, GI: 4995292), bp |
| | 118501 . . . 133259. |

| FEATURES | | Location/Qualifiers |
|---|---|---|
| | source | 1.. 14759 |
| | | /organism=“Homo sapiens” |
| | | /db_xref=“taxon : 9606” |
| | | /chromosome=“20” |
| | | /map=“20p12” |
| | | /clone=“RP5-859D4” |
| | | /clone_lib=“RPCI-5” |
| | gene | 2072..12634 |
| | | /gene=“BMP2” |
| | | /note=“BMP2A” |
| | | /db_xref=“LocusID:650” |
| | | /db_xref=“MIM:112261” |
| | exon | 2072..2387 |
| | | /gene=“BMP2” |
| | | /number=1 |
| | exon | 3632..3984 |
| | | /gene=“BMP2” |
| | | /number=2 |
| | CDS | /join(3639..3984, 11757..12601) |

/gene=“BMP2”
/note=“BMP2 exons defined by
comparison to mRNA

sequence (NM_001200)”

/codon_start=1
/product=“bone morphogenetic
protein 2 precursor”
/protein_id=“NP_001191.1”
/db_xref=“GI:4557369”

TABLE 3

| | | nucleotide | | |
|---|---|---|---|---|
| | nucleotide | position | | |
| | position | relative | | |
| nucleotide | relative to | to SEQ | position | amino acid |
| change | SEQ ID NO 1 | AL035668 | in gene | change |
| A to G | −2047 | 116454 | promoter | |
| T to C | −1136 | 117365 | promoter | |
| (ATTT)n | −901 | 117600 | promoter | |
| C to T | −638 | 117863 | promoter | |
| C to T | −568 | 117933 | promoter | |
| T to C | −72 | 118429 | promoter | |
| G to A | 70 | 118570 | promoter | |
| A insertion | 368 | 118868 | promoter | |
| A to G | 420 | 118920 | promoter | |
| A to G | 472 | 118972 | promoter | |
| G to C | 1464 | 119964 | 5′ utr | |
| G to A | 1722 | 120222 | 5′ utr | |
| C to G | 1914 | 120414 | 5′ utr | |
| A to C | 2536* | 121036 | intron 1 | |
| C to T | 2866 | 121366 | intron 1 | |
| G to T | 3145 | 121645 | intron 1 | |
| T to G | 3747 | 122247 | exon 2 | serine to |
| | | | | alanine |
| A to G | 3899* | 122399 | exon 2 | |
| G to T | 3918 | 122418 | exon 2 | alanine to |
| | | | | serine |
| A to G | 4181 | 122681 | intron 2 | |
| G to A | 4244 | 122744 | intron 2 | |
| A to T | 4359 | 122859 | intron 2 | |
| G to A | 4435 | 122935 | intron 2 | |
| T insertion | 4712 | 123212 | intron 2 | |
| T to A | 5041 | 123541 | intron 2 | |
| C to T | 5048 | 123548 | intron 2 | |
| G to A | 5787 | 124287 | intron 2 | |
| G to A | 6217 | 124717 | intron 2 | |
| G to A | 7111* | 125611 | intron 2 | |
| A to T | 7162 | 125662 | intron 2 | |
| T to C | 7781* | 126281 | intron 2 | |
| A to G | 7828 | 126328 | intron 2 | |
| C to T | 7874 | 126374 | intron 2 | |
| G to C | 8035* | 126535 | intron 2 | |
| A to C | 8083 | 126583 | intron 2 | |
| T to G | 8463 | 126963 | intron 2 | |
| G to A | 9013* | 127513 | intron 2 | |
| G to A | 9082 | 127582 | intron 2 | |
| G to T | 10631 | 129131 | intron 2 | |
| A to G | 10841 | 129341 | intron 2 | |
| A to T | 11980* | 130480 | exon 2 | arginine to |
| | | | | serine |
| C to T | 12571 | 131071 | exon 2 | |
| A to C | 12845* | 131345 | 3′ utr | |
| T to C | 13066 | 131566 | 3′ utr | |
| A to G | 13209* | 131709 | 3′ utr | |
| C to A | 13296* | 131796 | 3′ utr | |
| 4 bp | 13533- | 132033- | 3′ utr | |
| deletion | 13536 | 132036 | | |

*known in SNP databases

### Example 3

### Direct Sequencing of the BMP2 Nucleic Acid Sequence Reveals Other Polymorphisms.

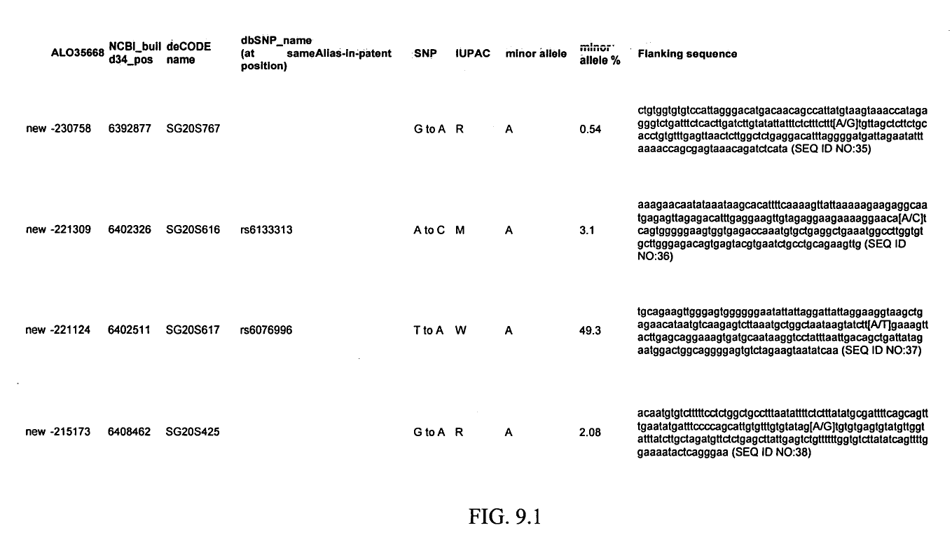

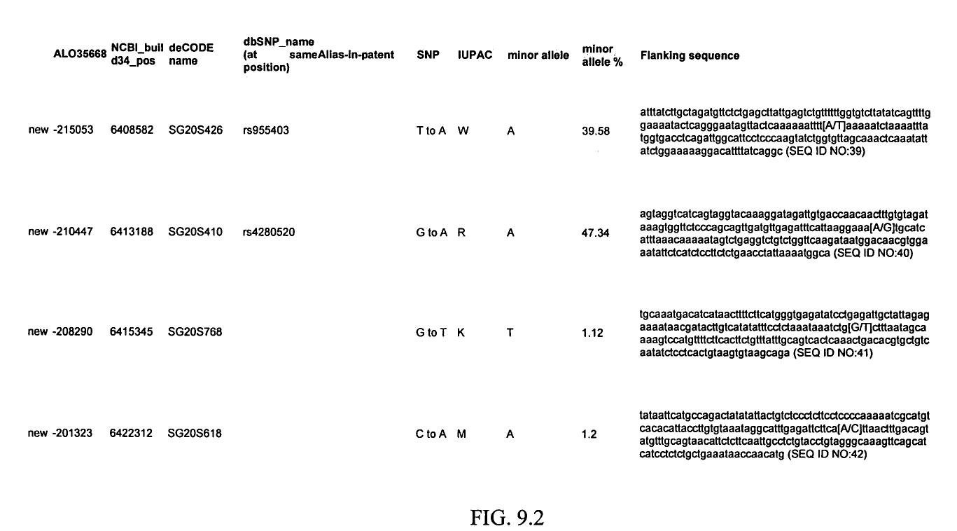

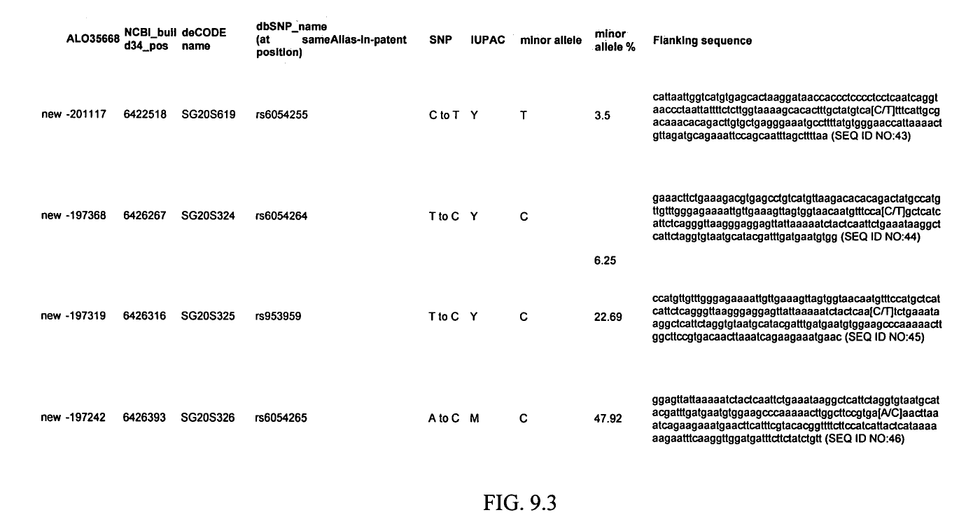

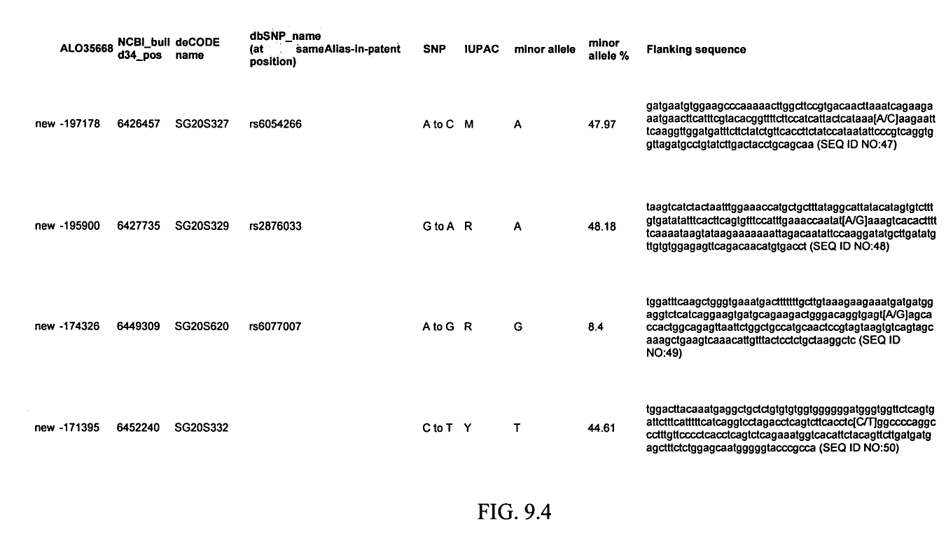

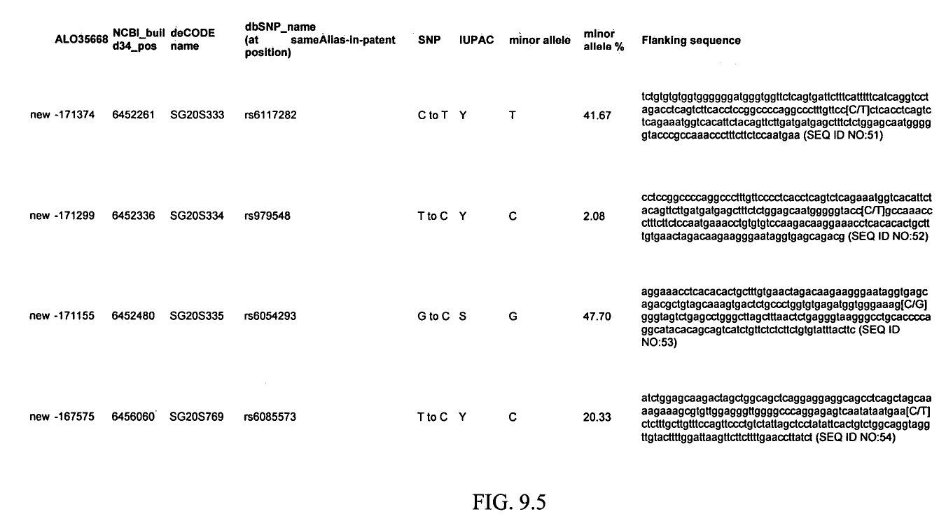

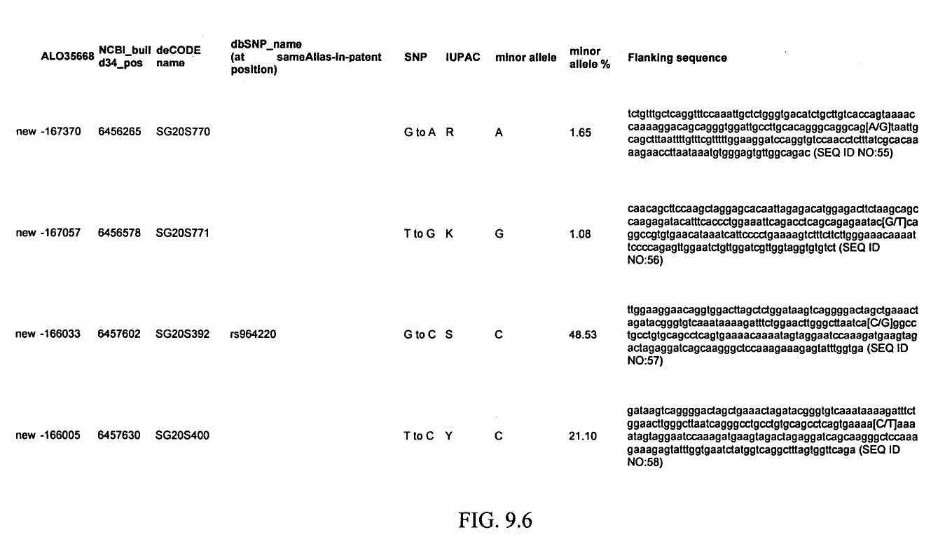

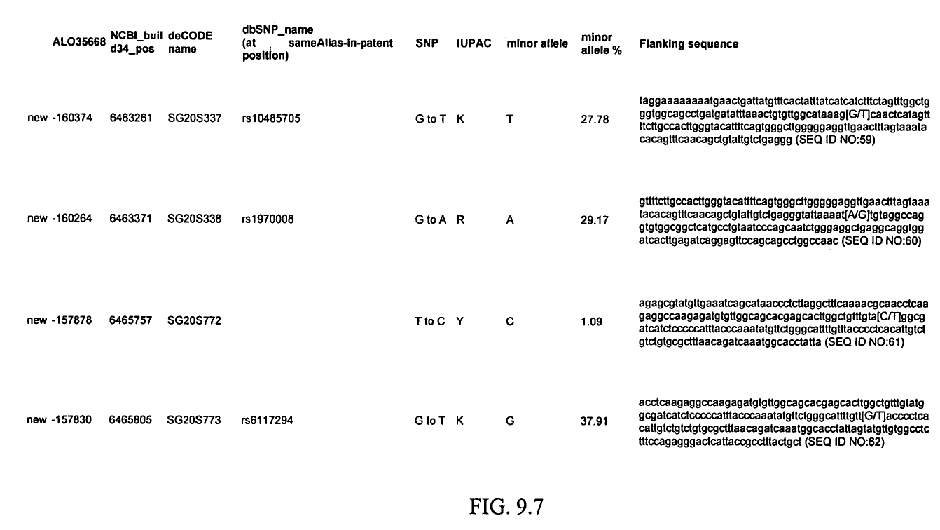

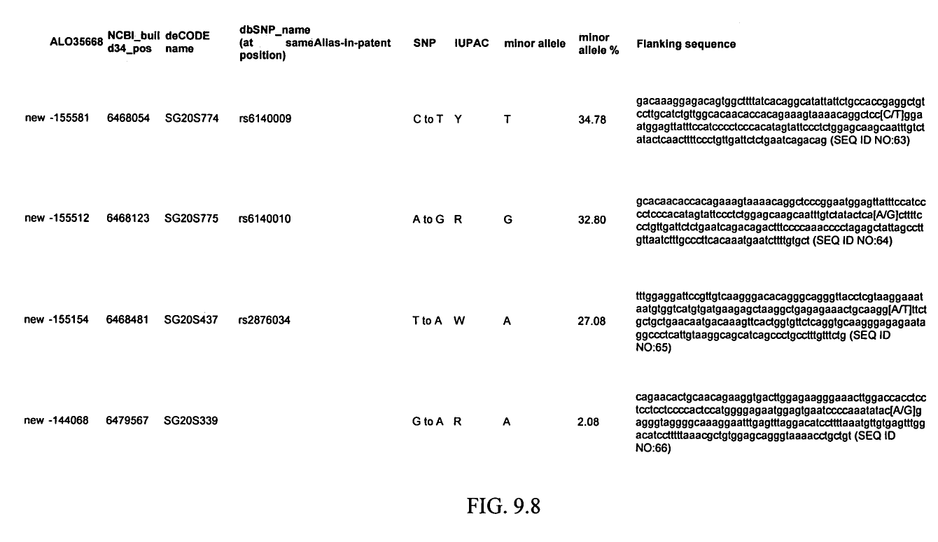

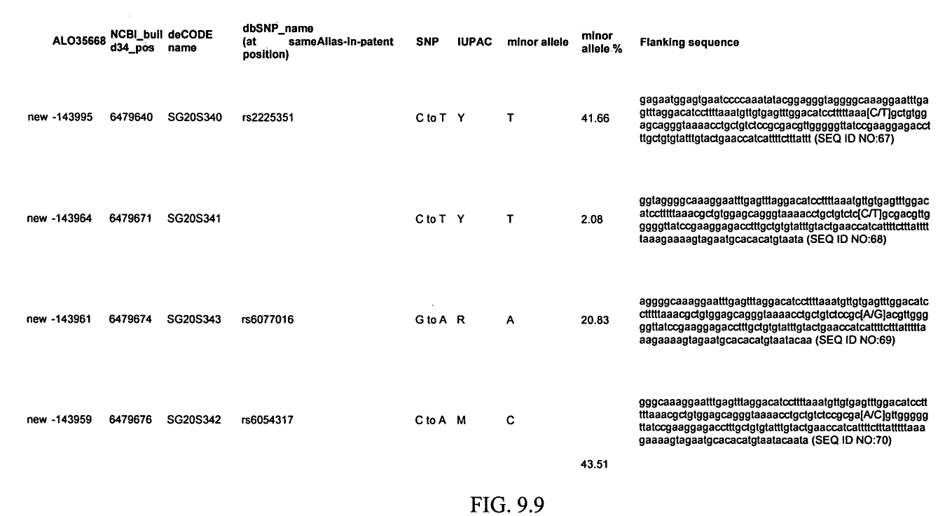

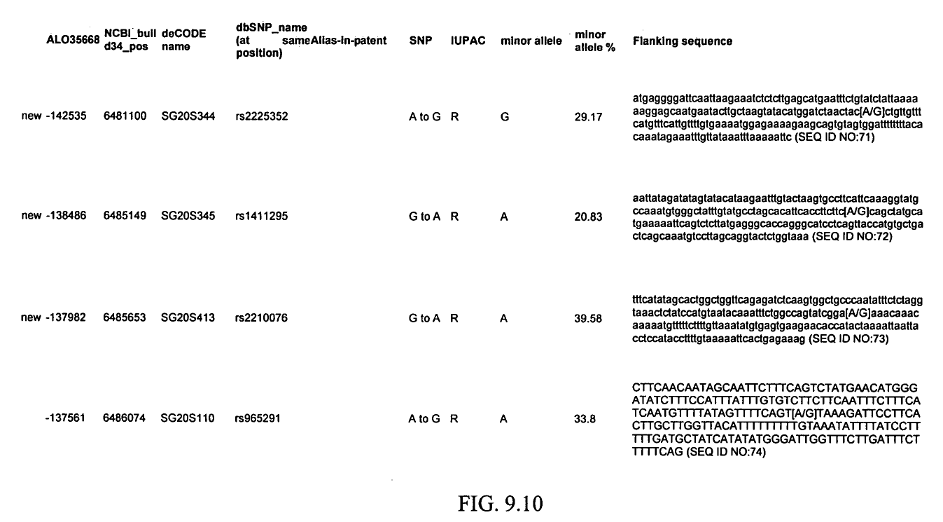

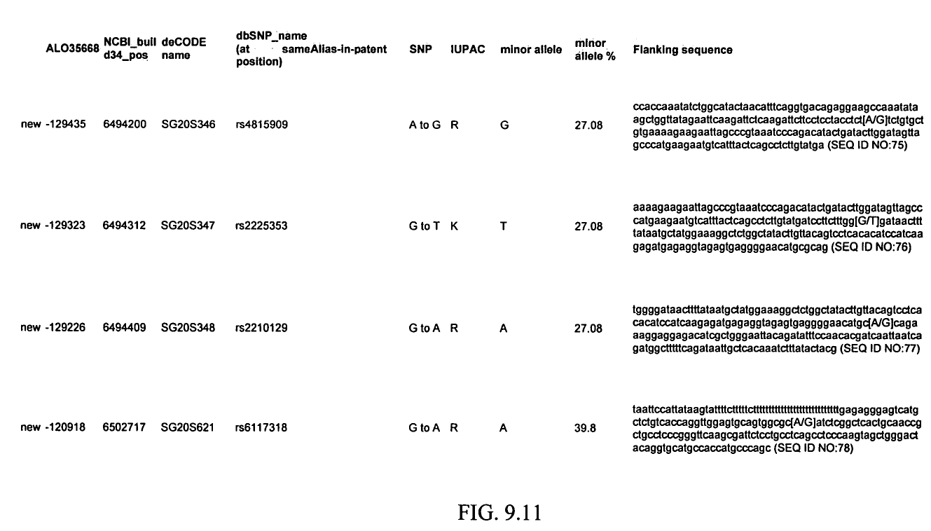

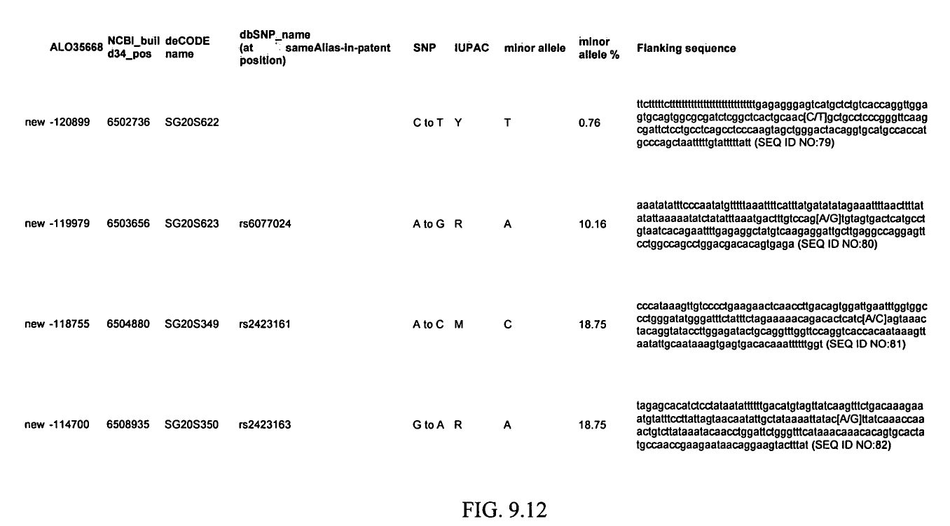

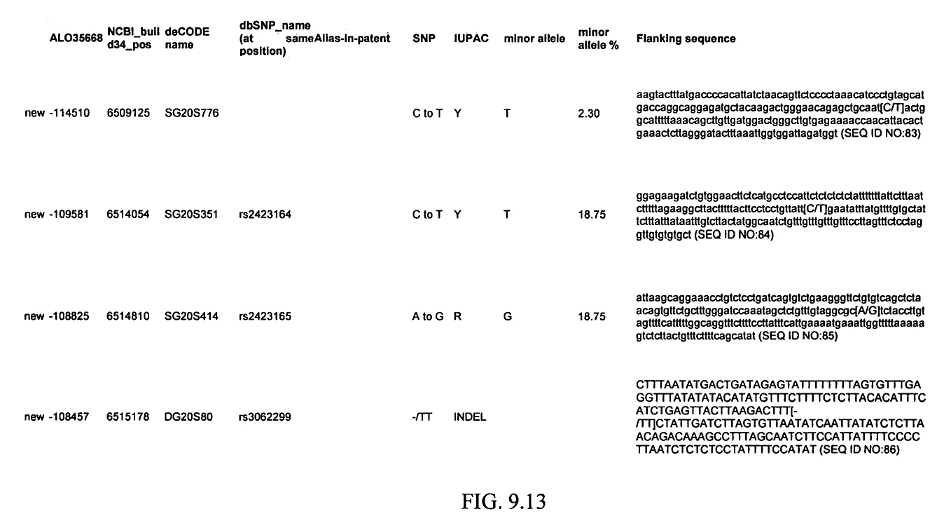

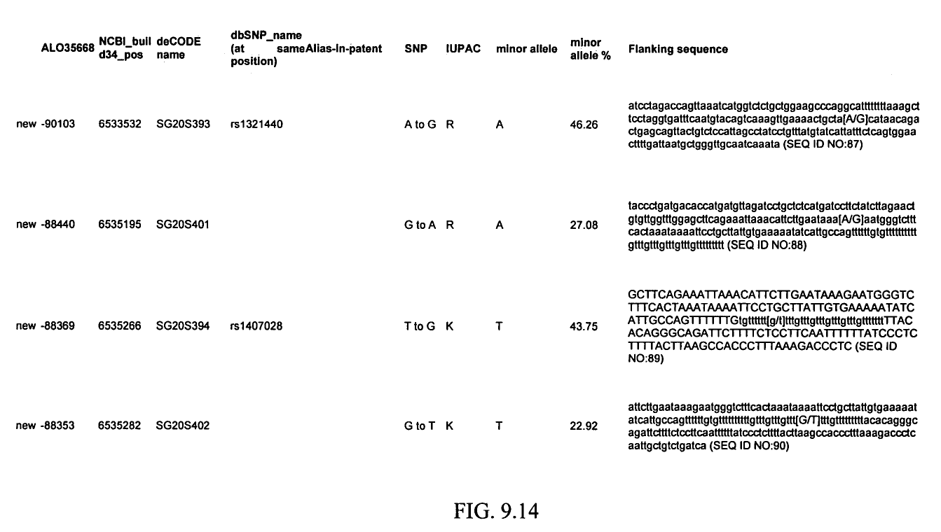

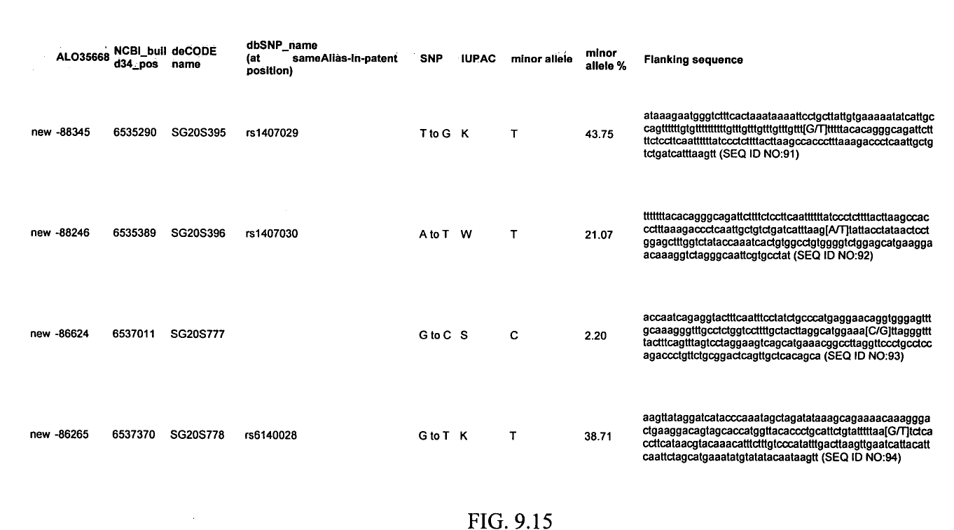

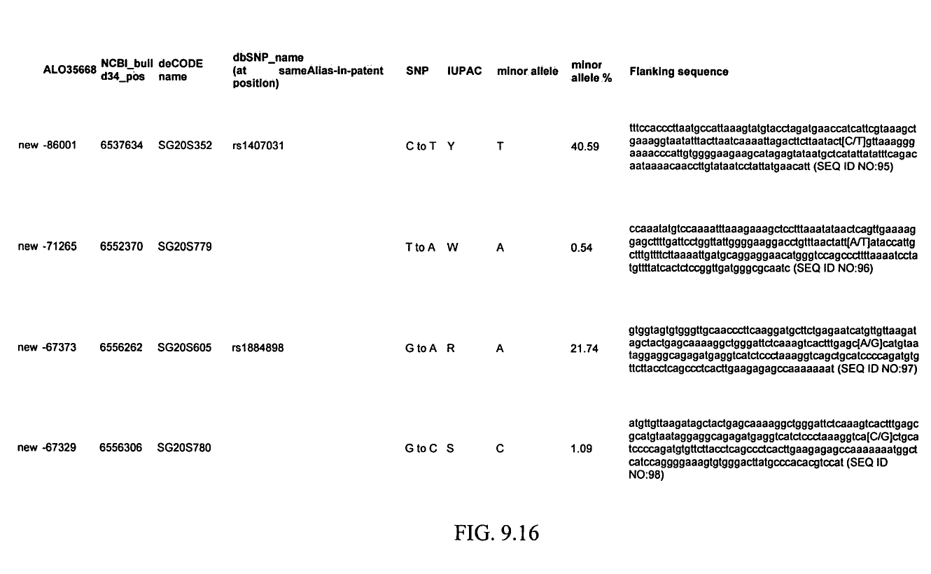

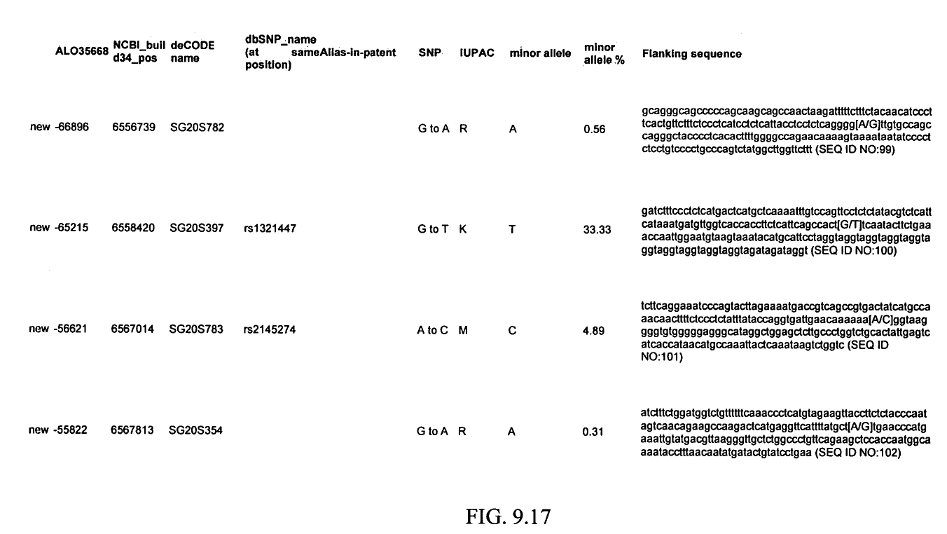

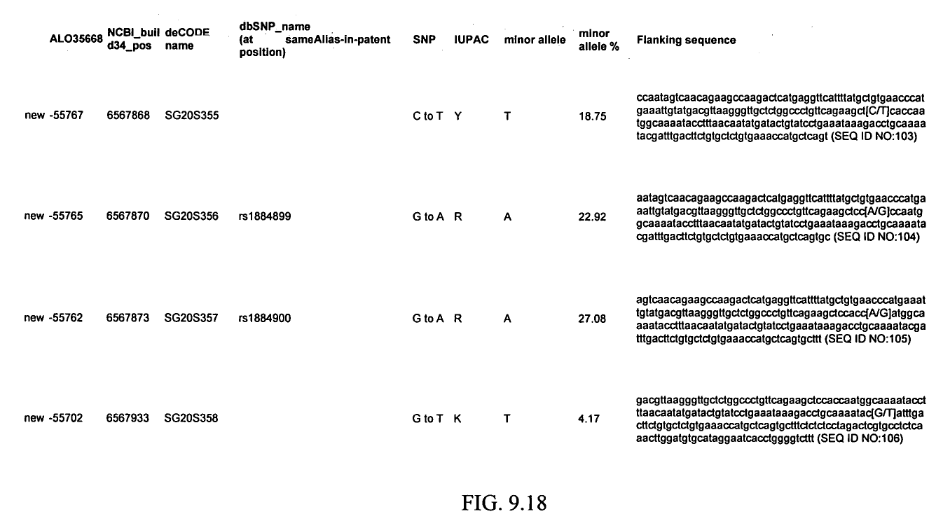

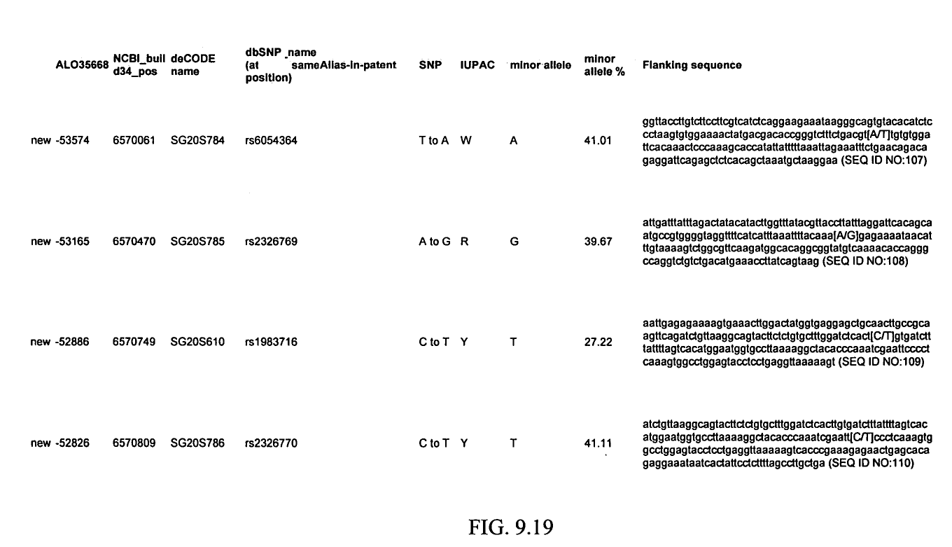

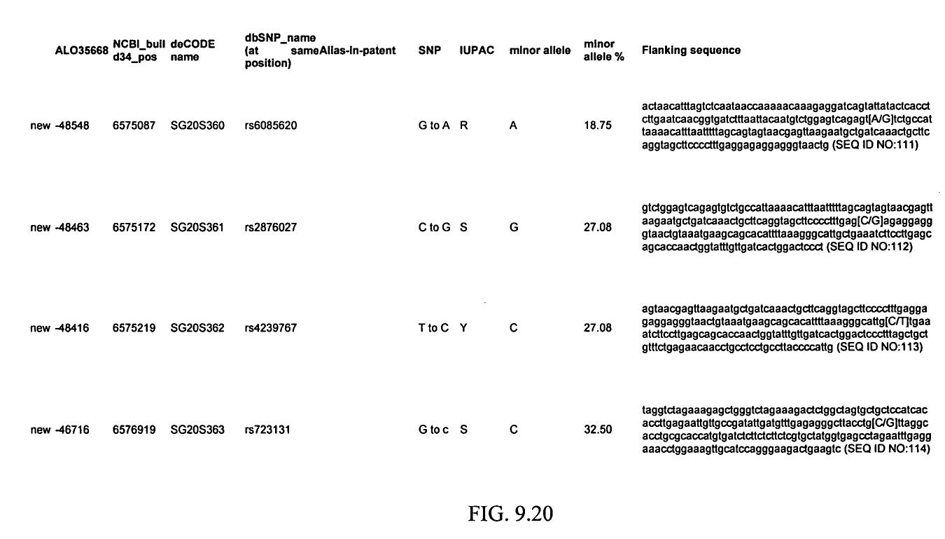

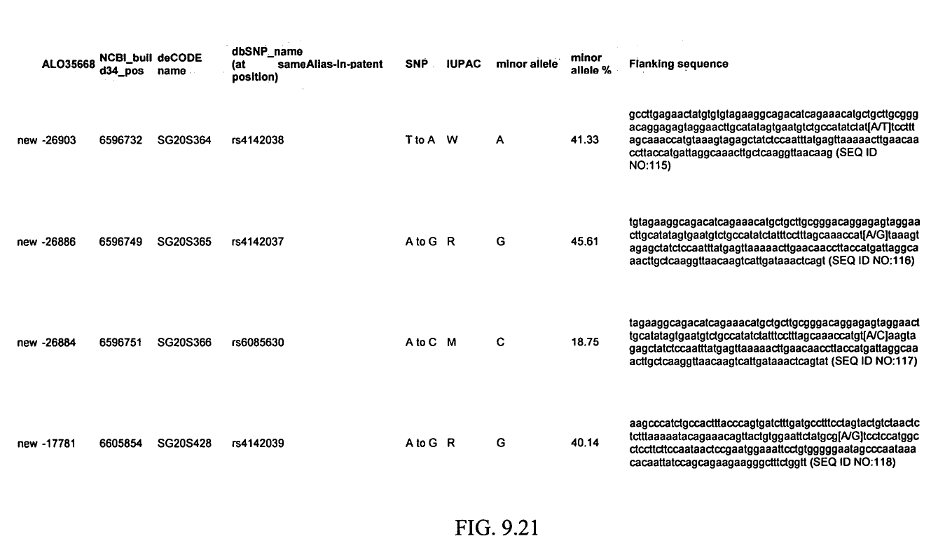

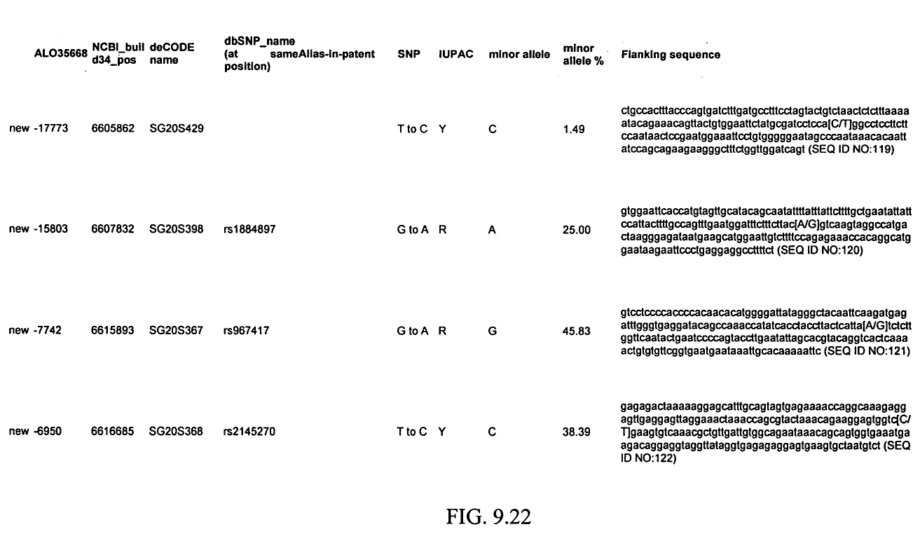

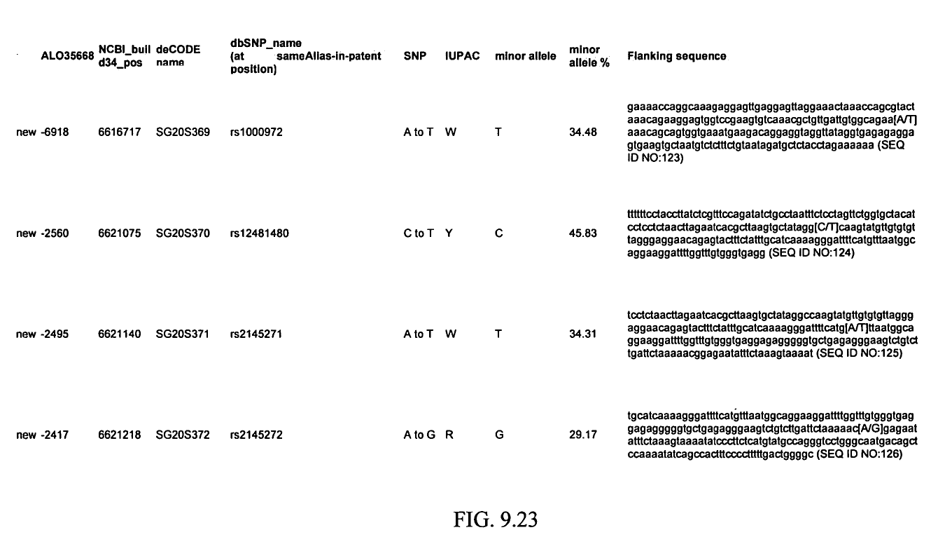

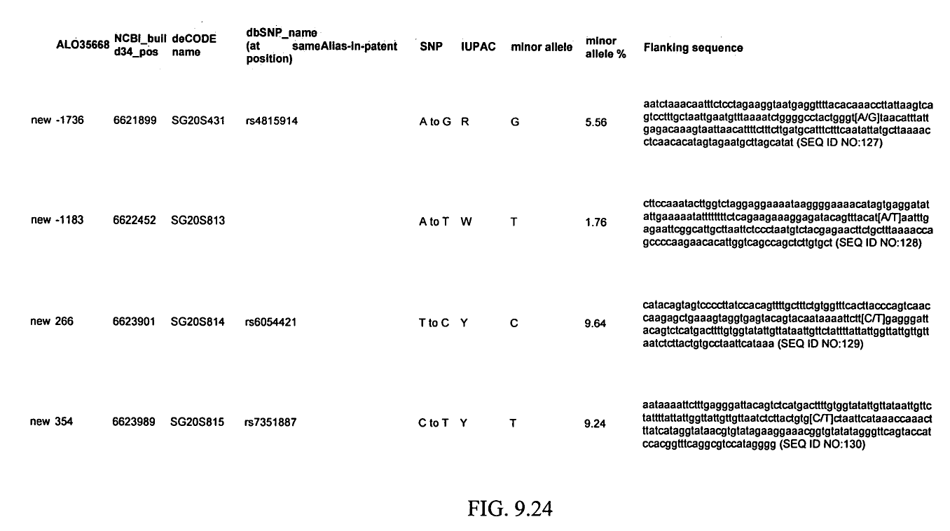

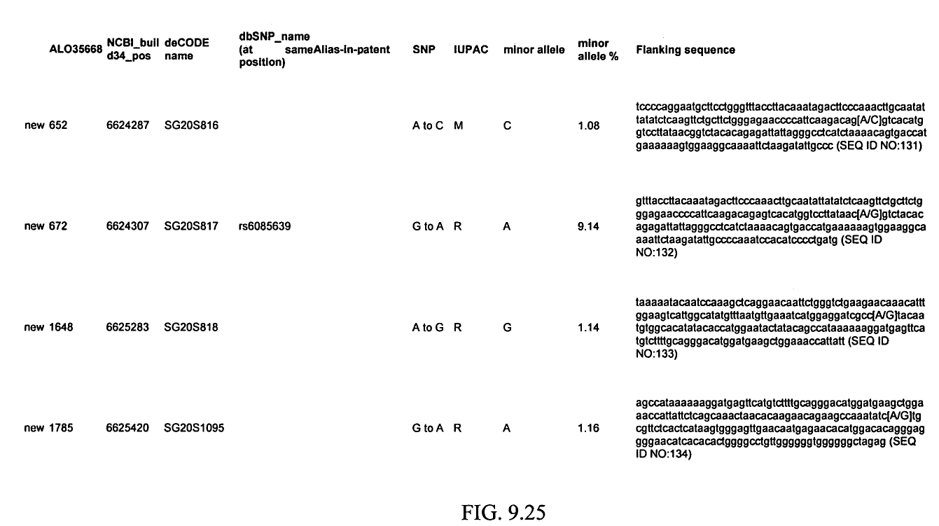

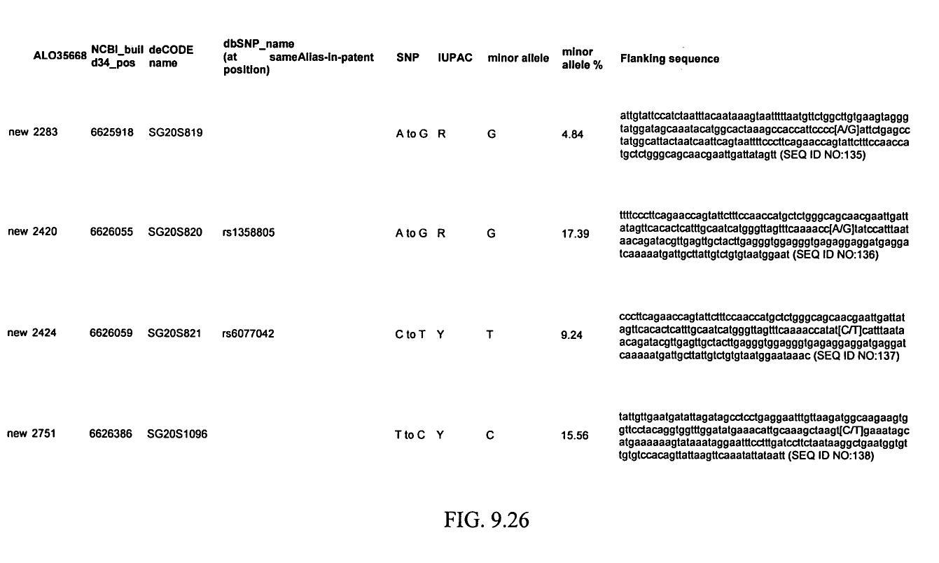

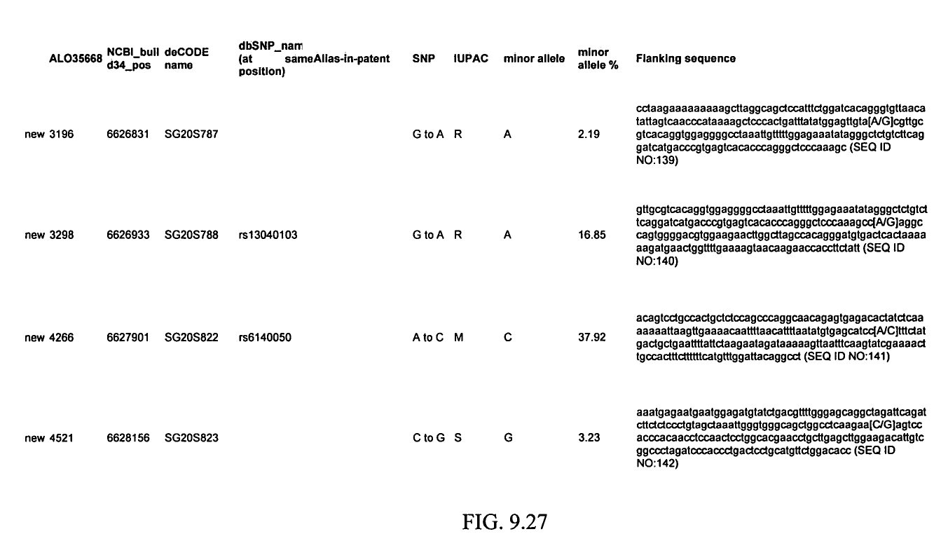

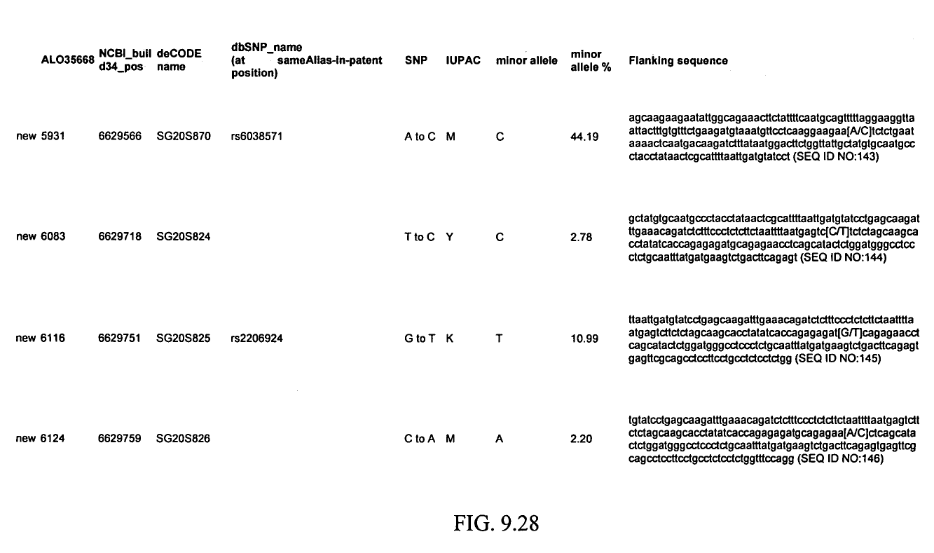

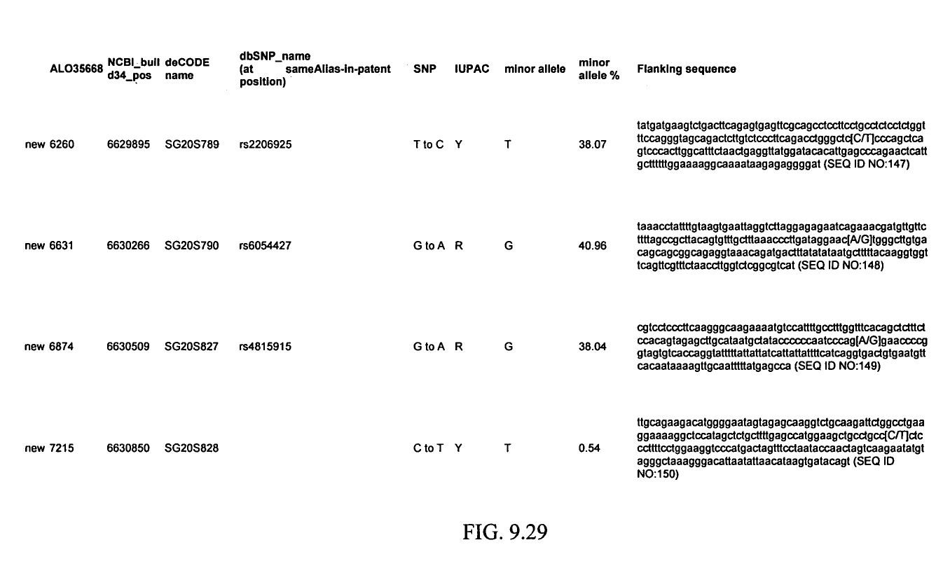

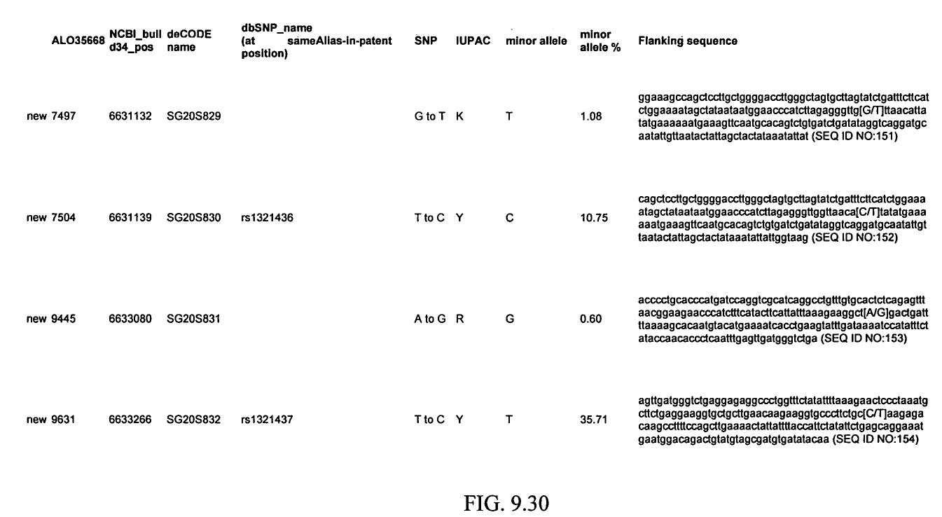

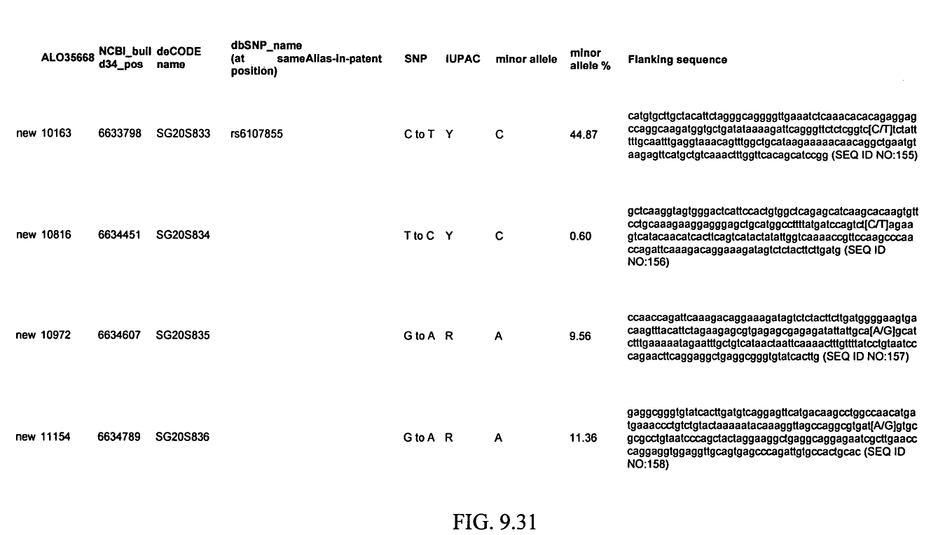

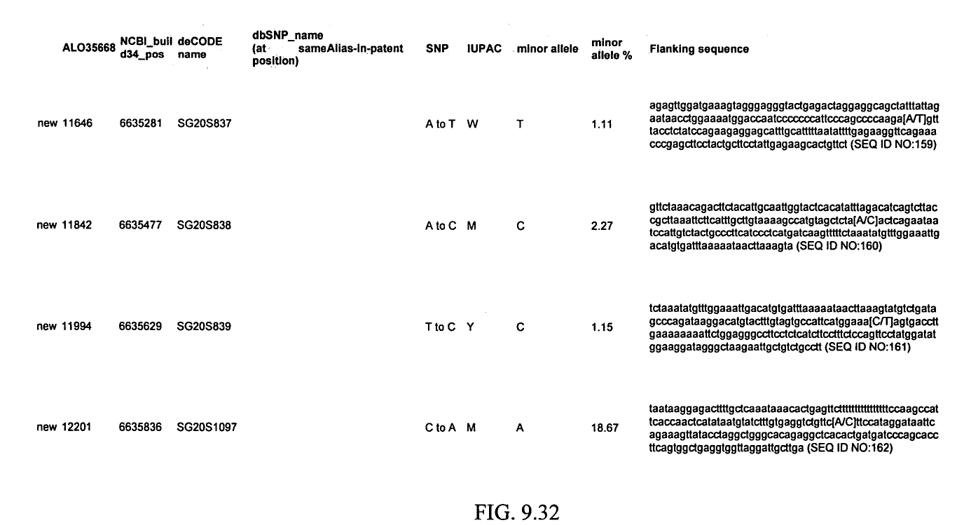

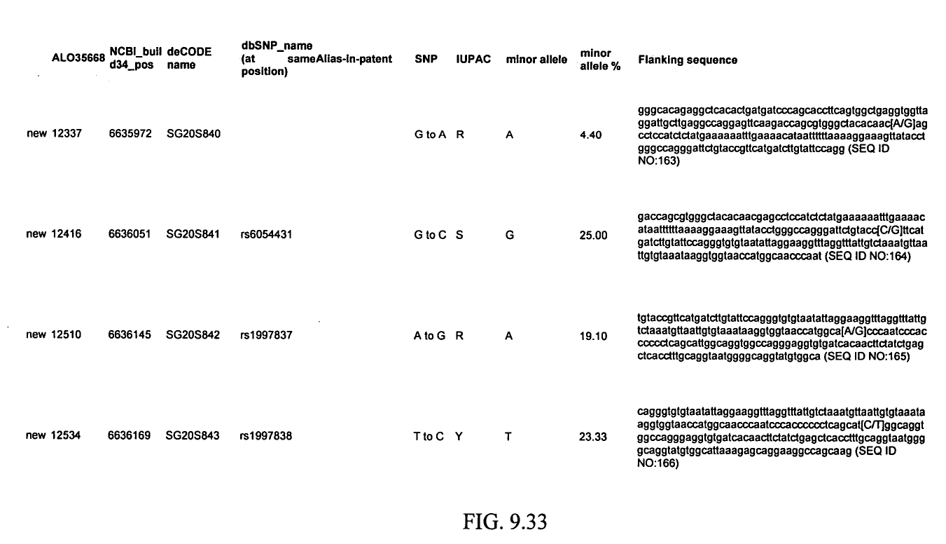

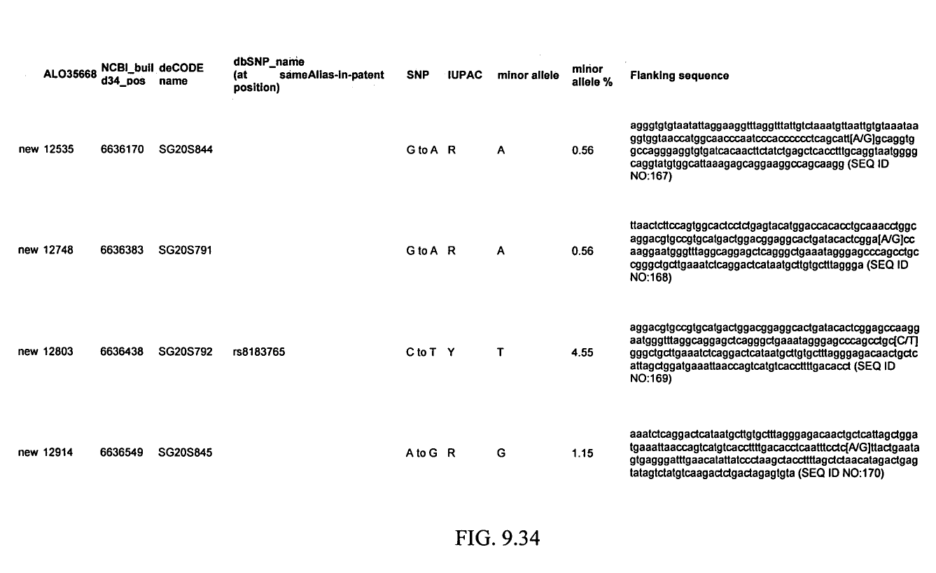

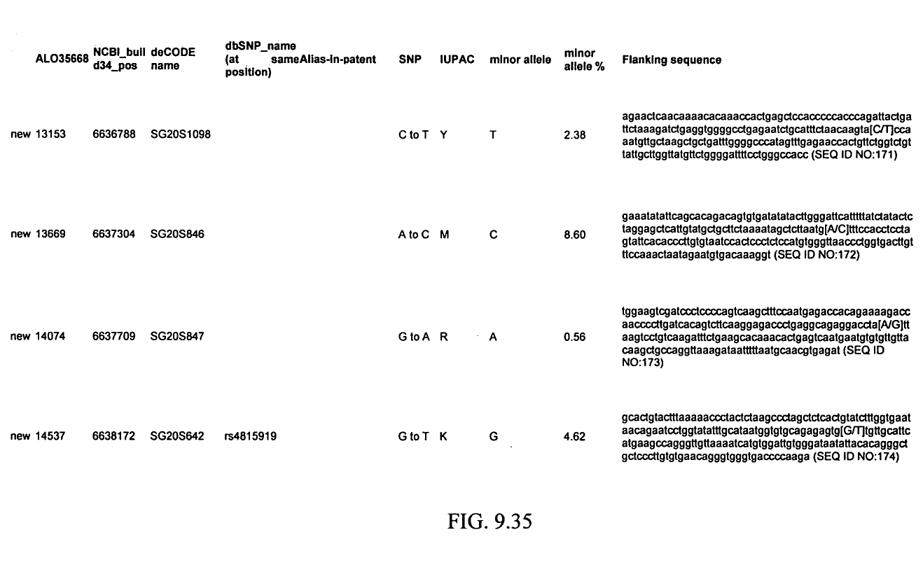

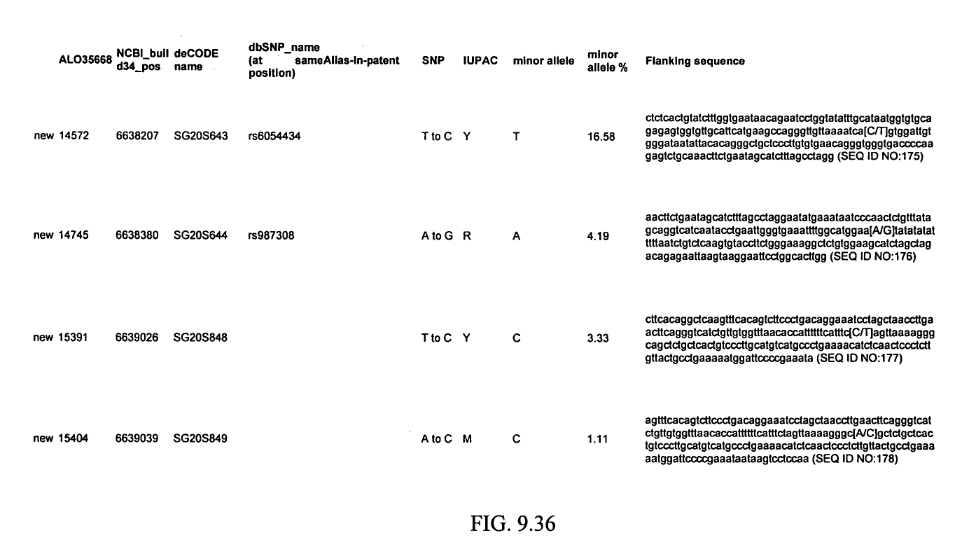

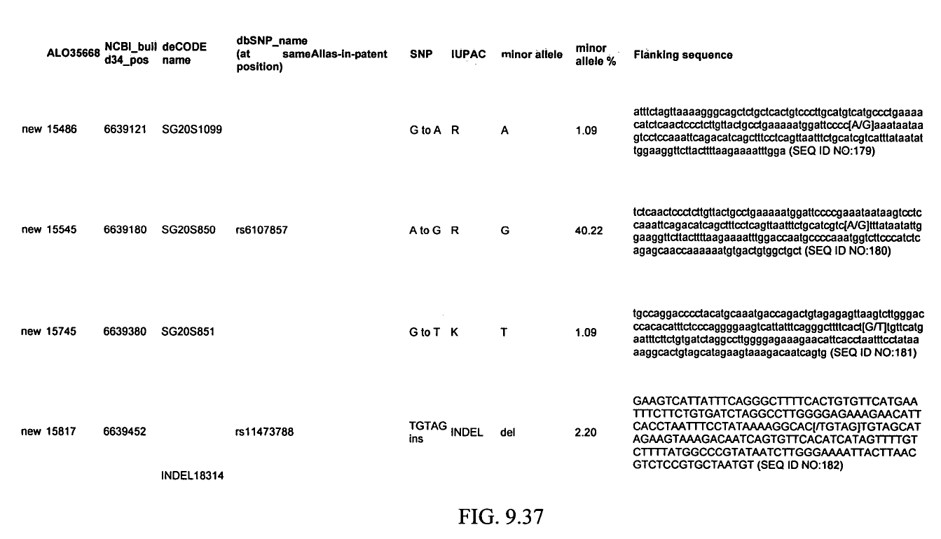

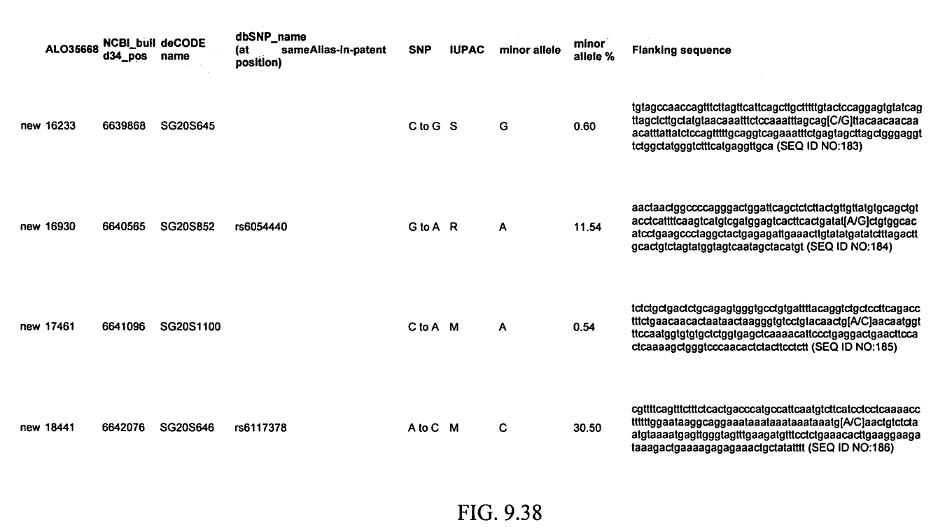

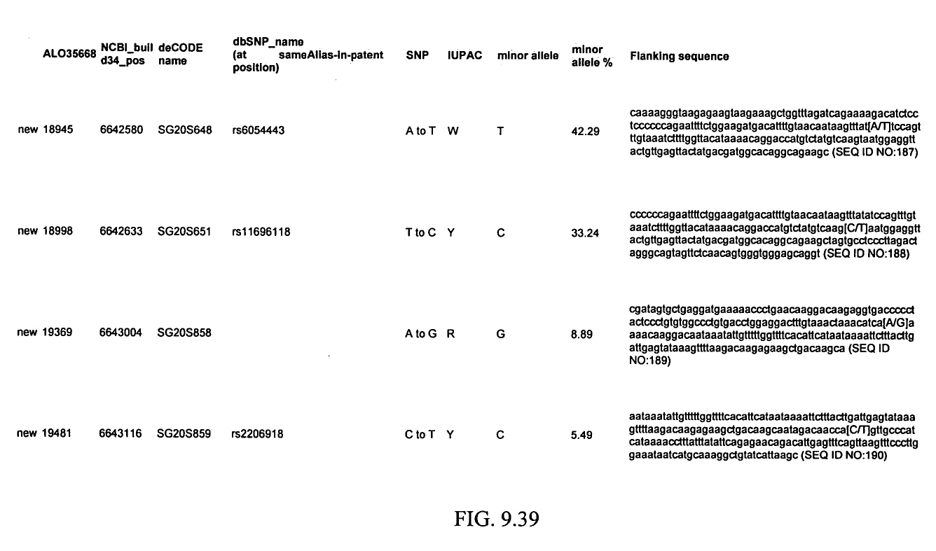

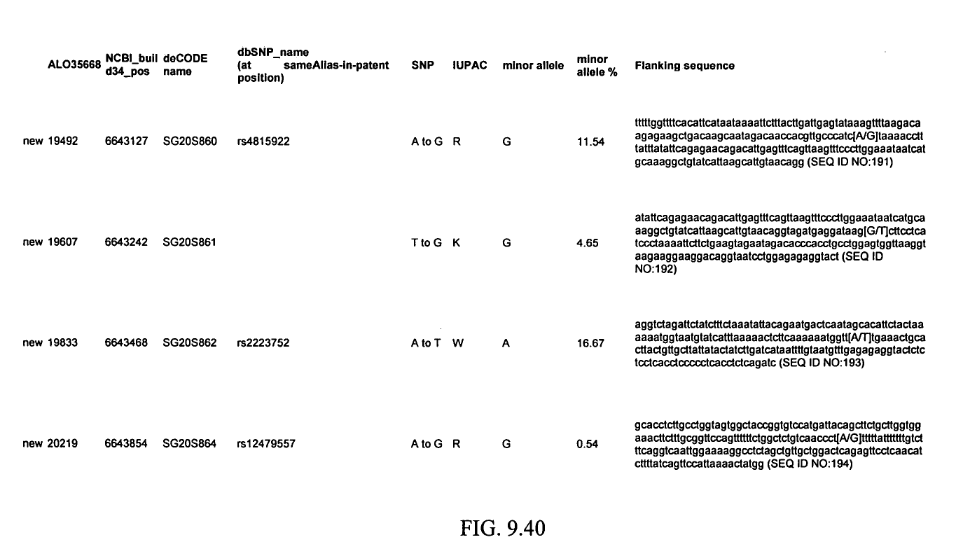

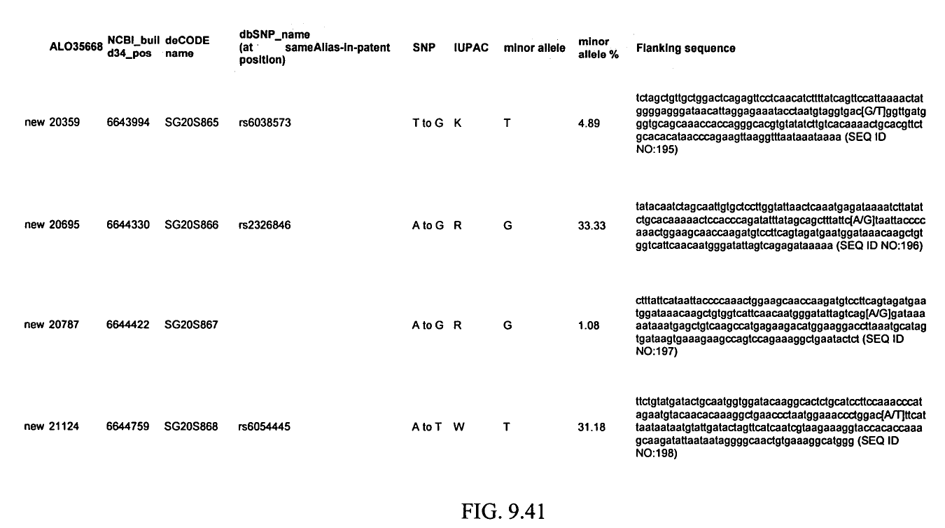

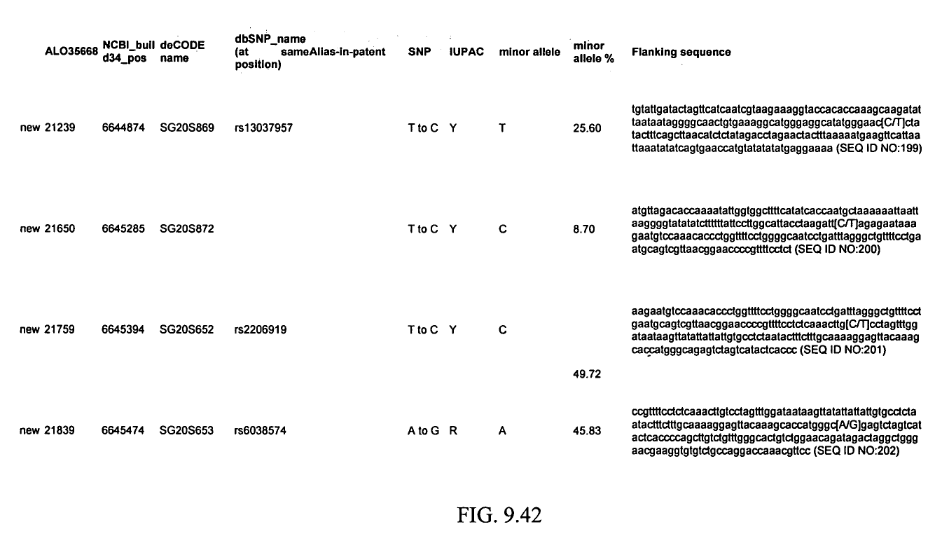

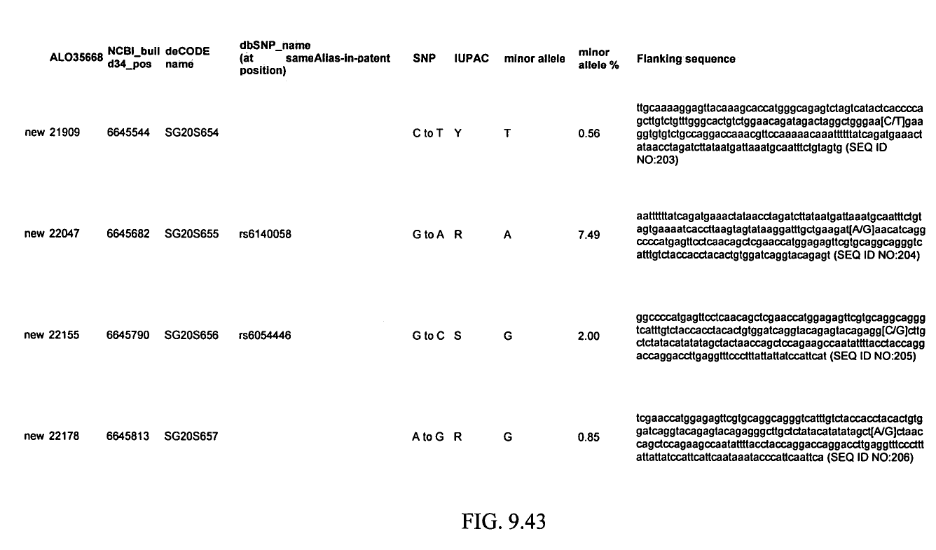

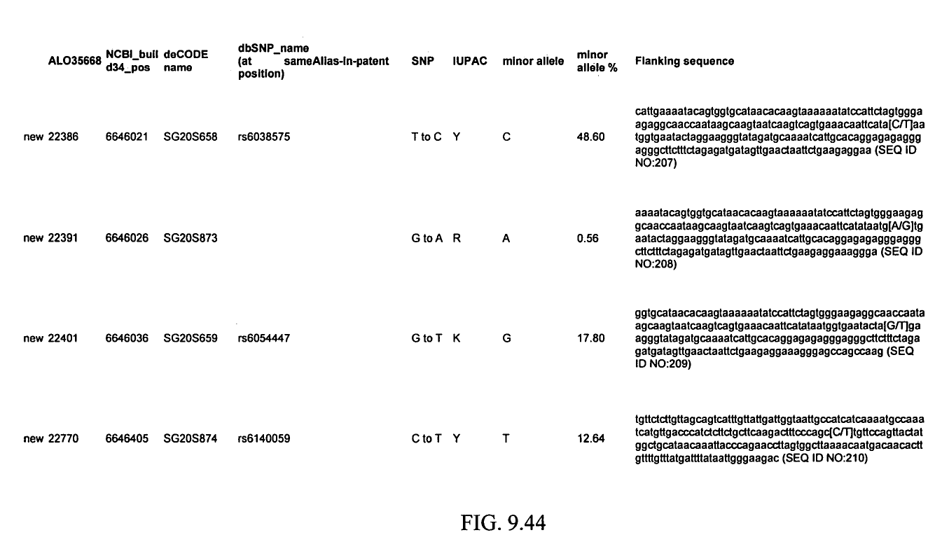

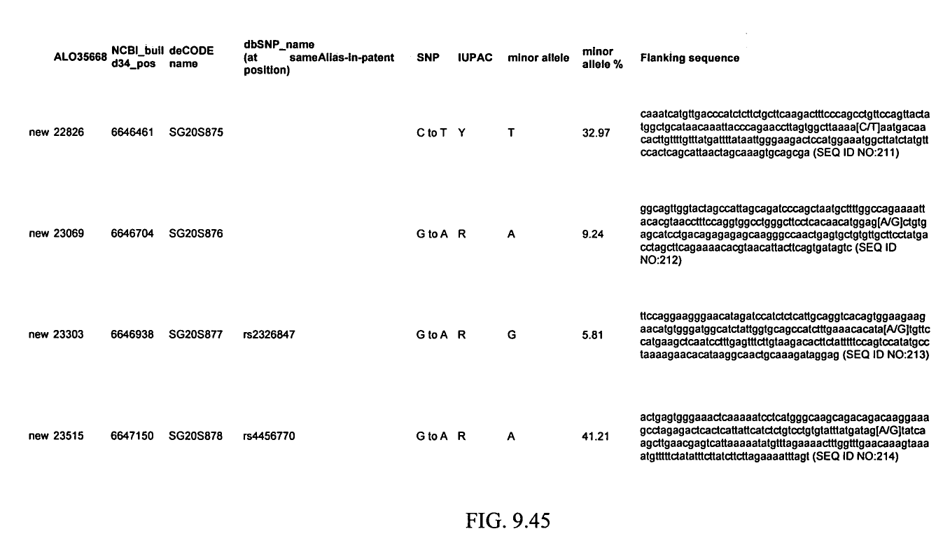

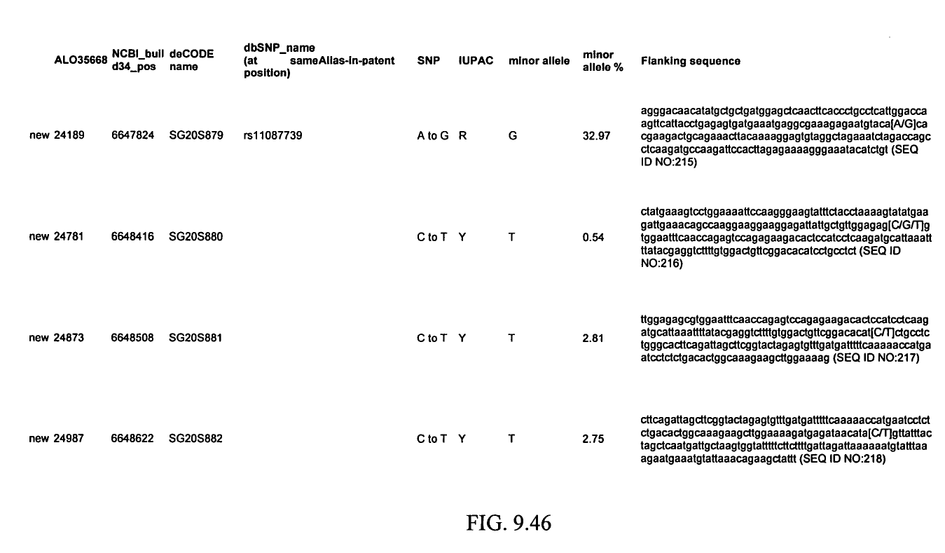

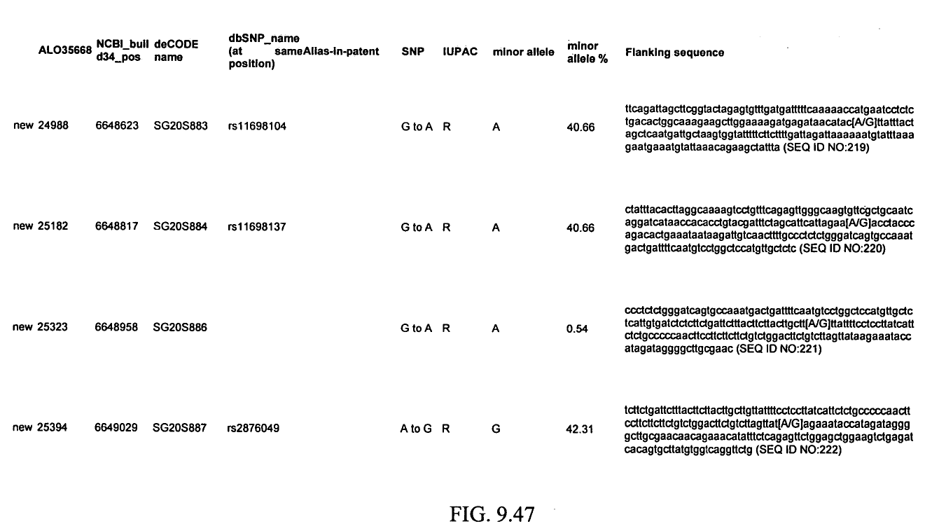

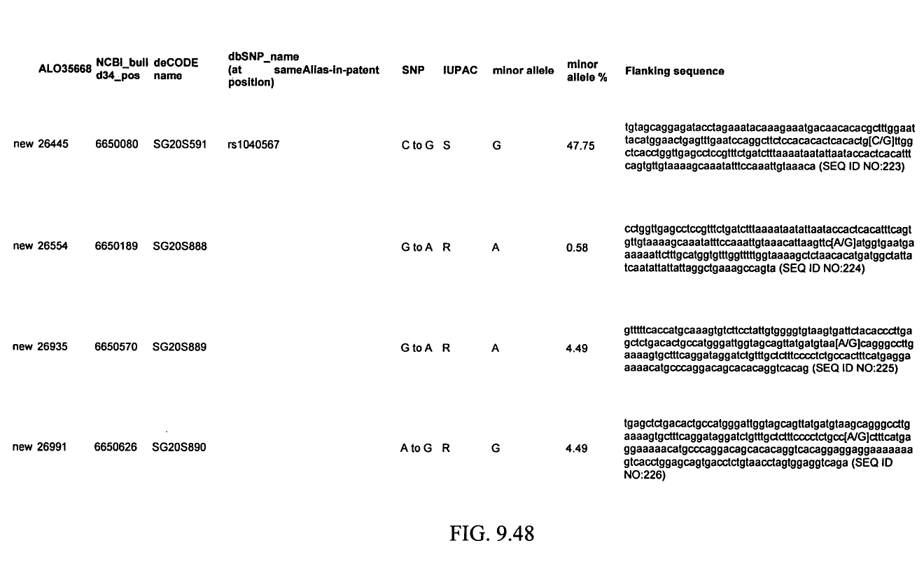

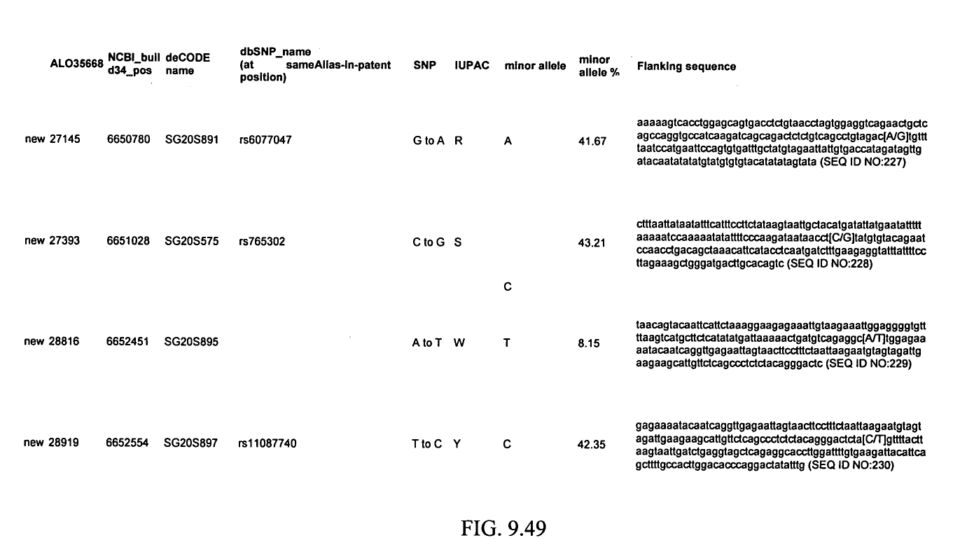

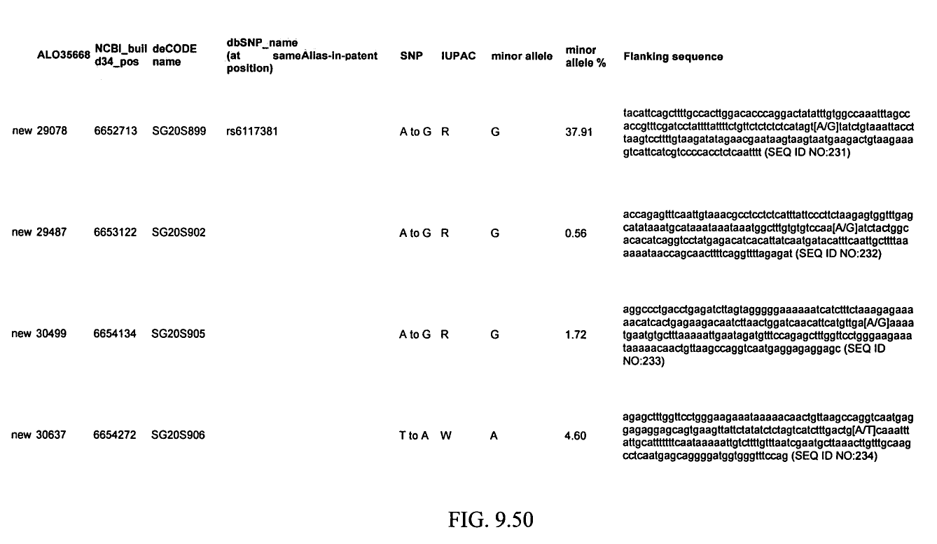

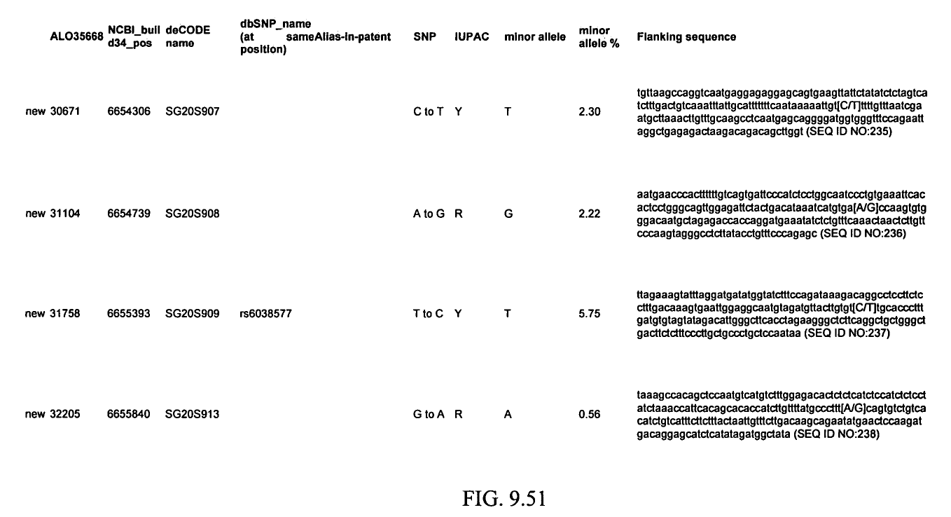

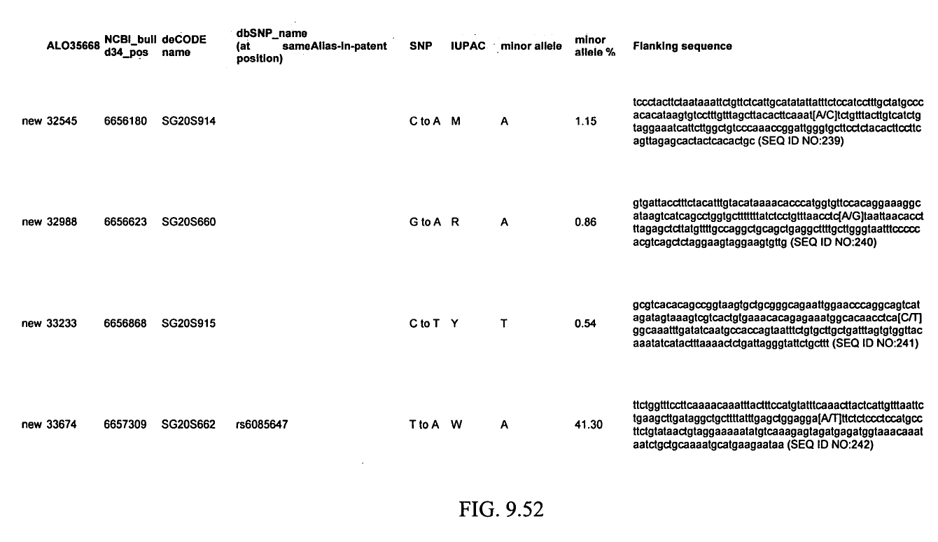

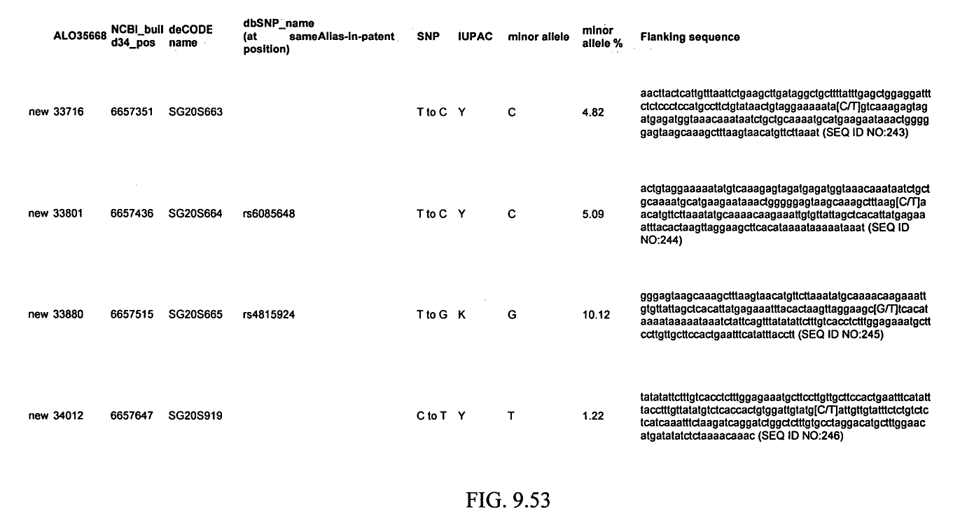

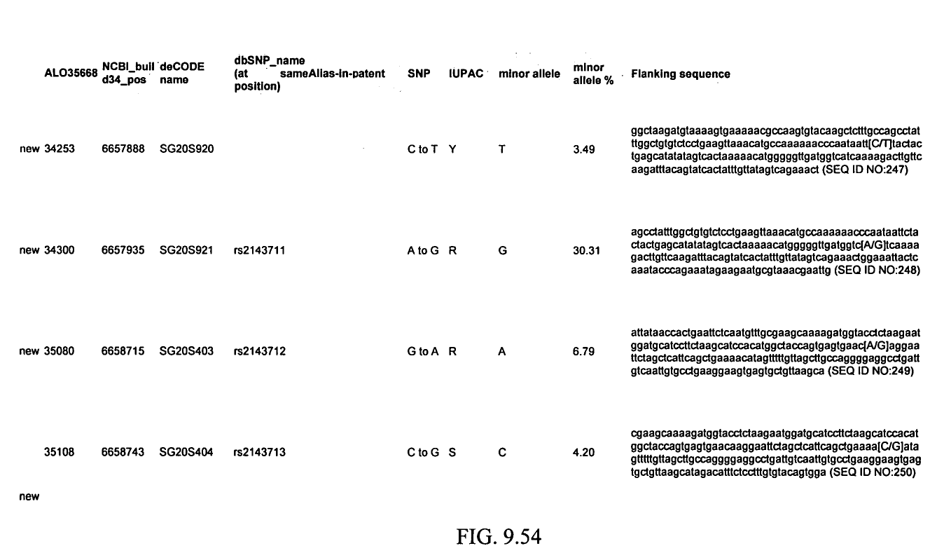

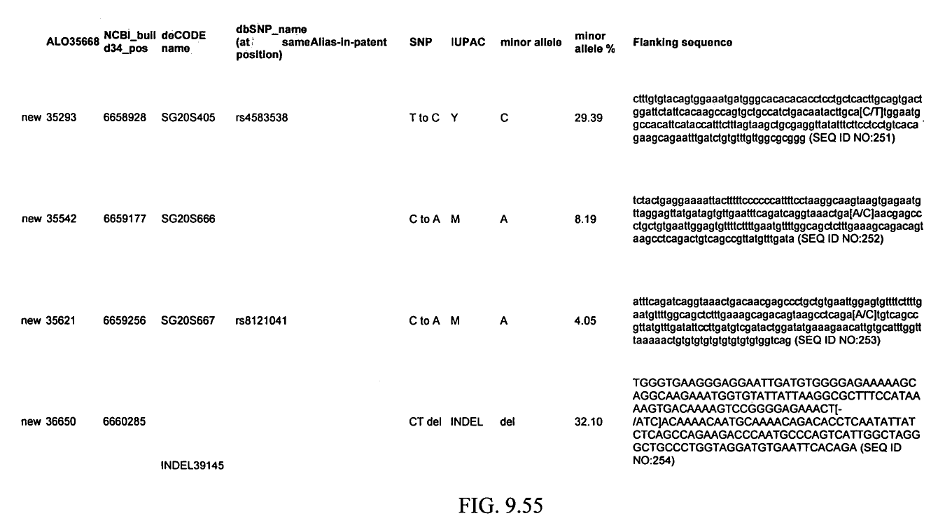

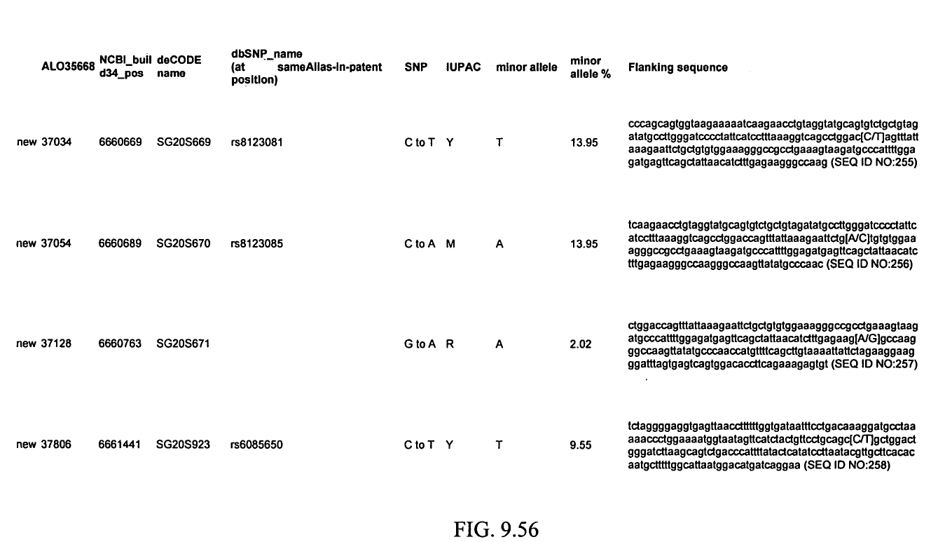

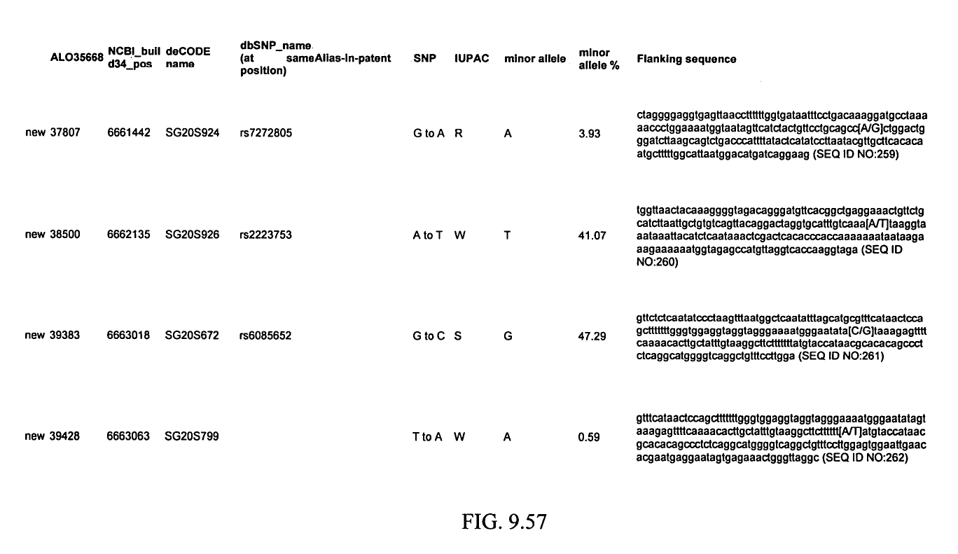

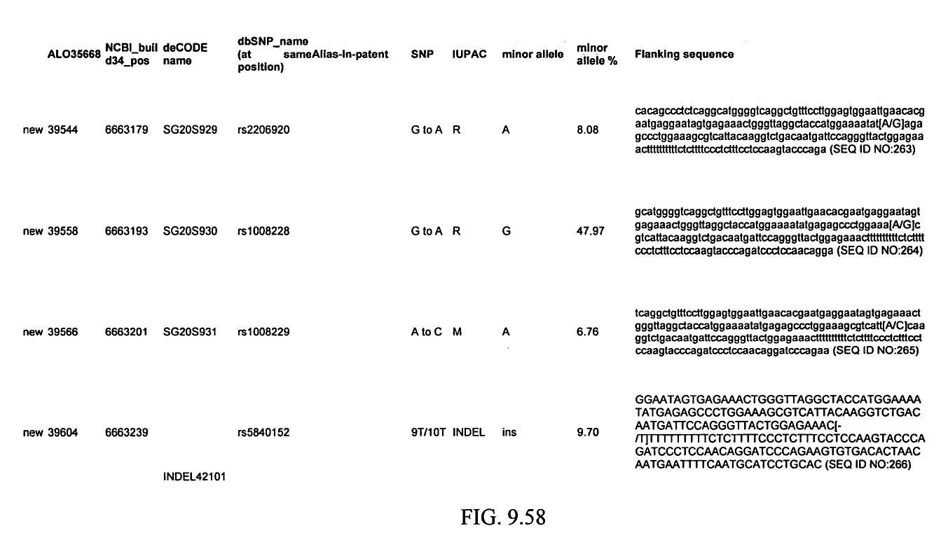

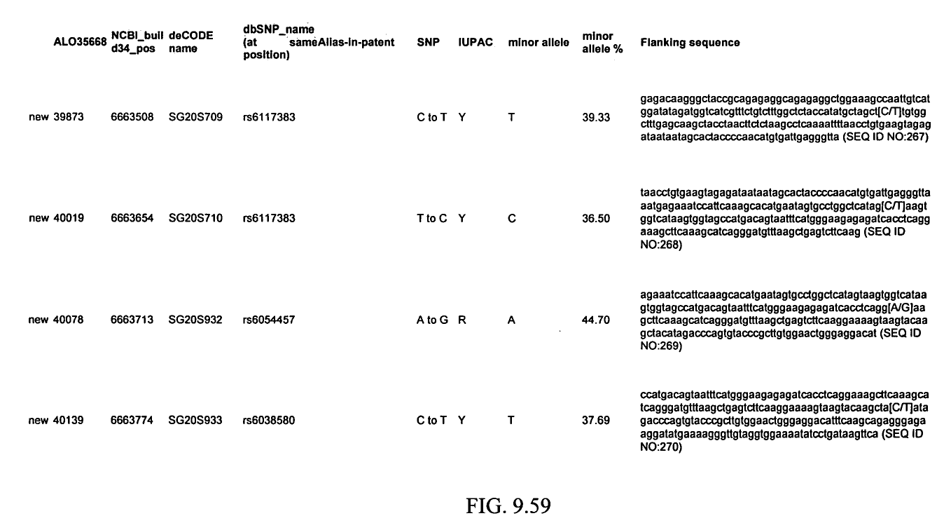

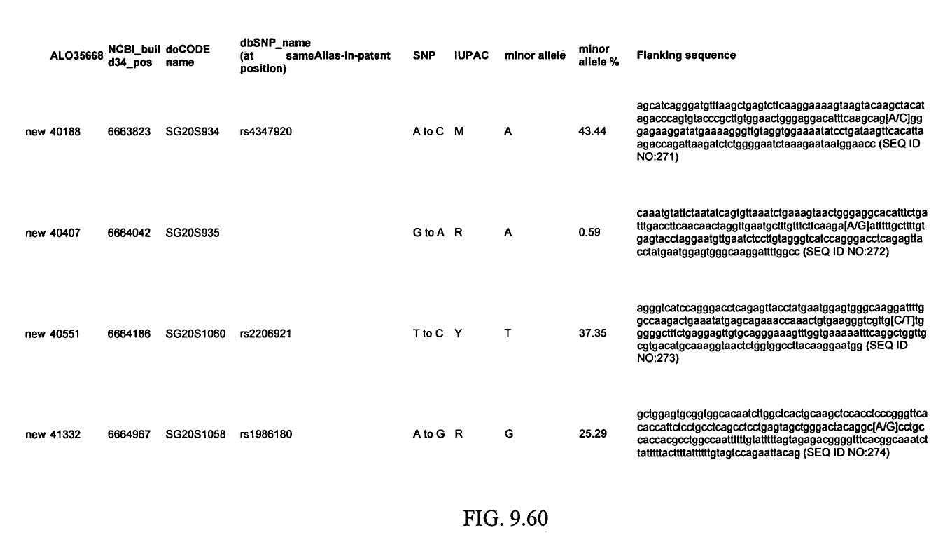

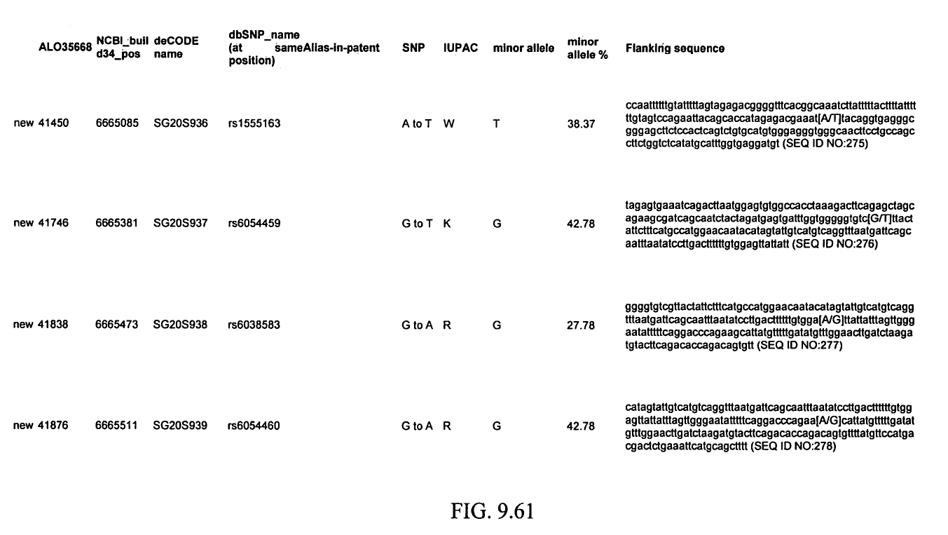

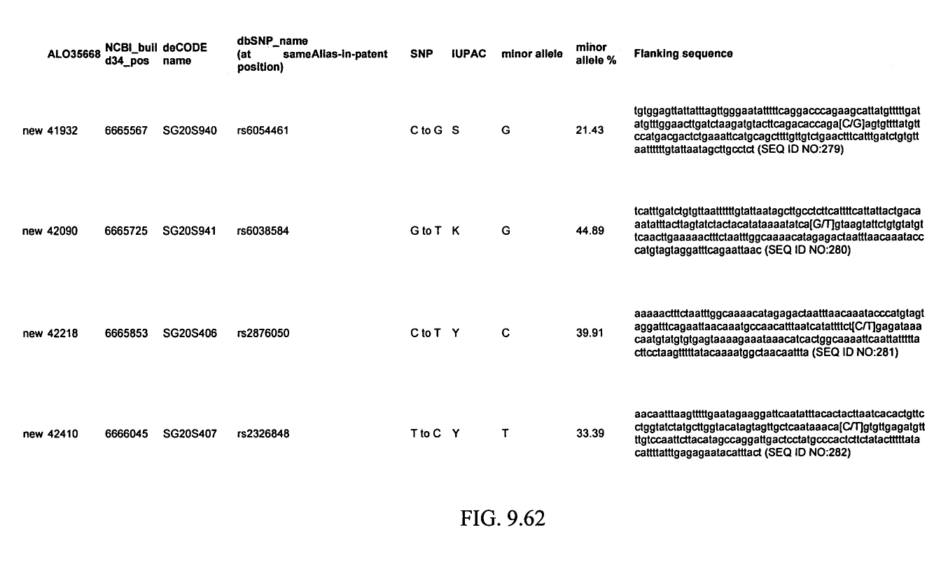

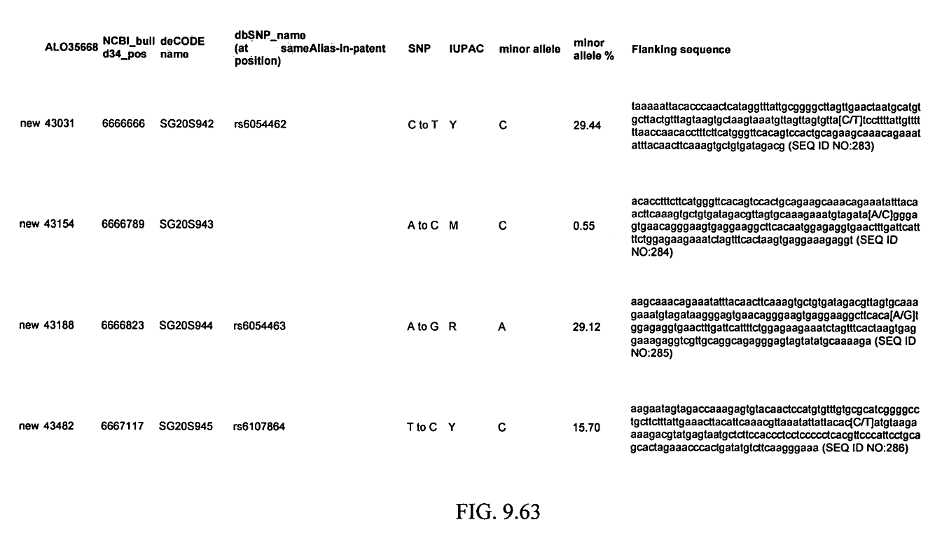

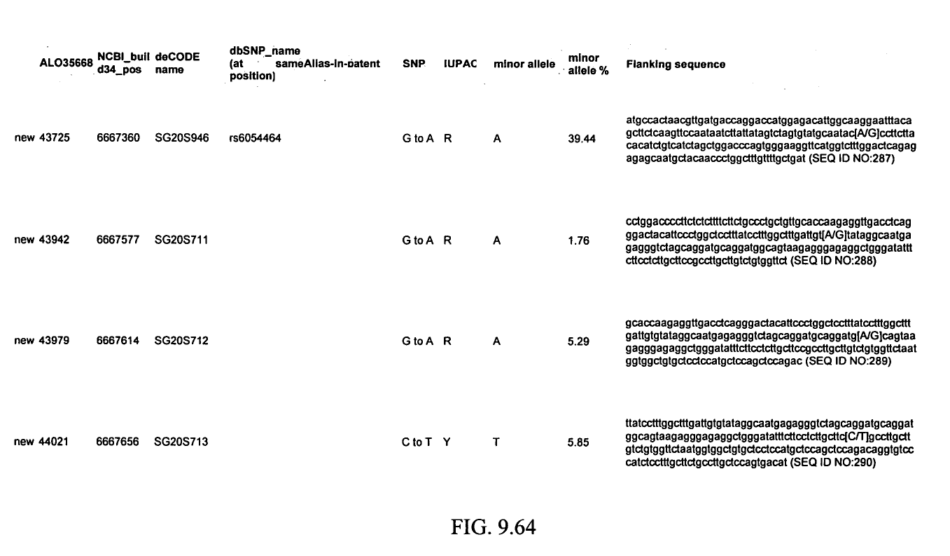

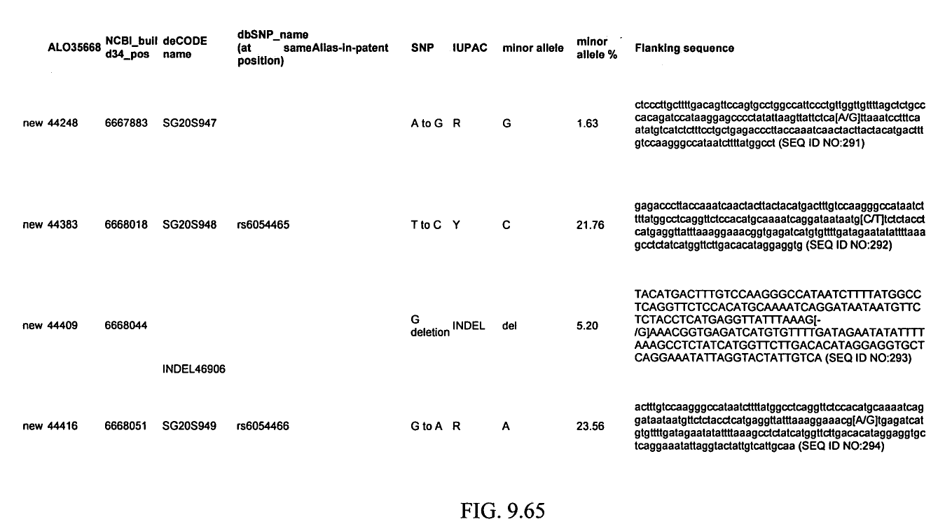

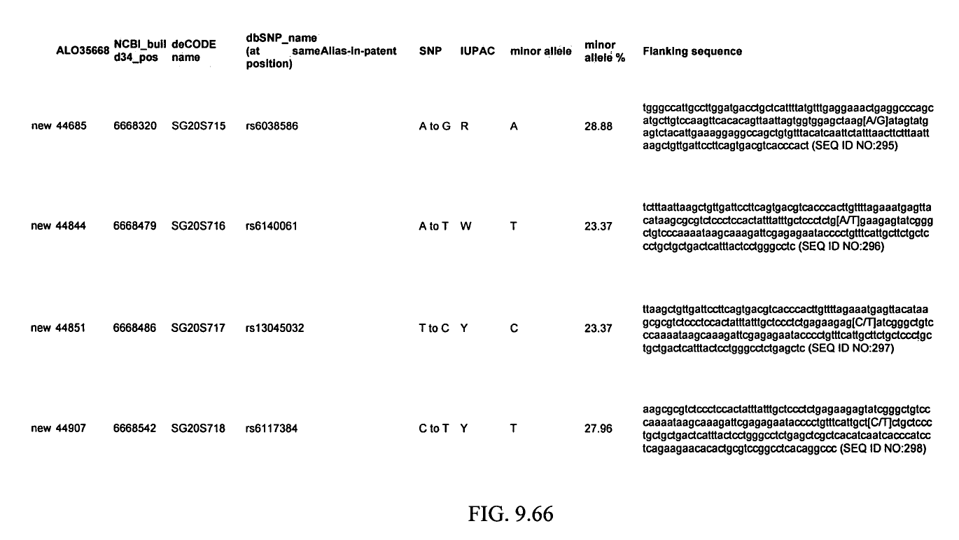

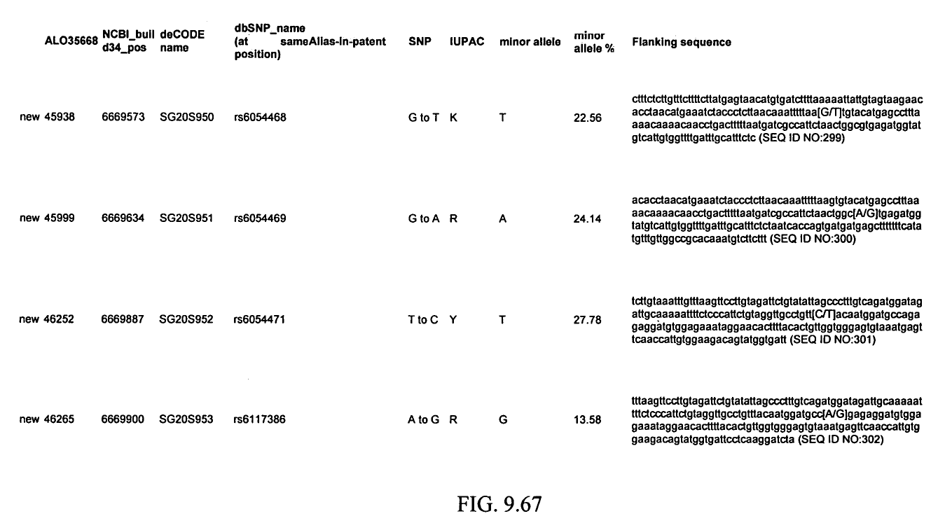

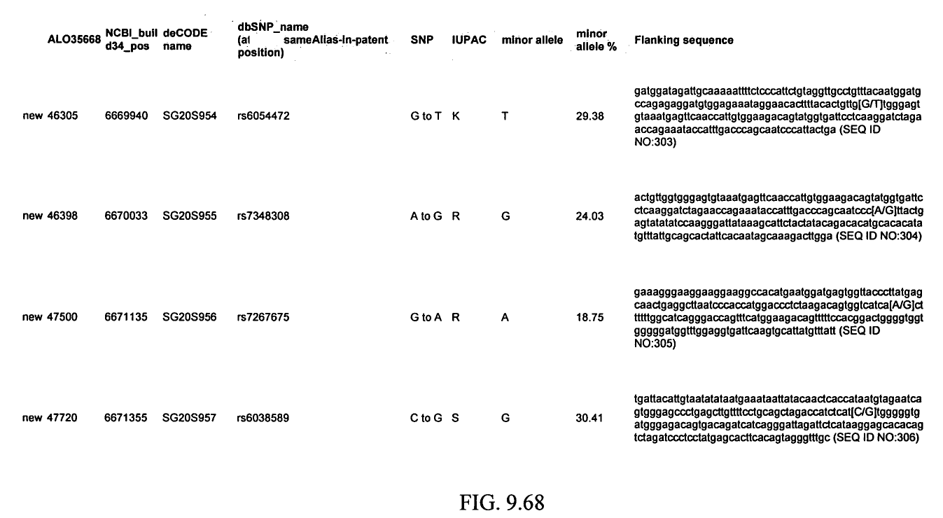

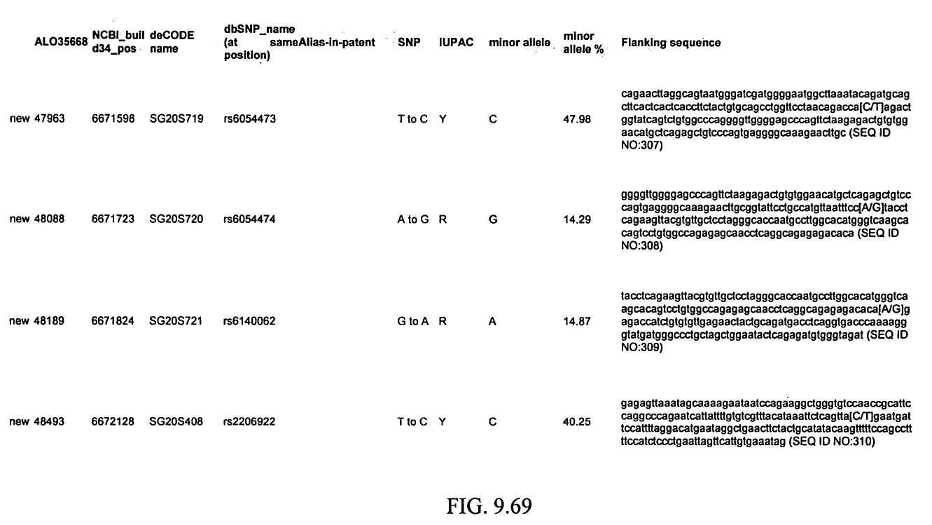

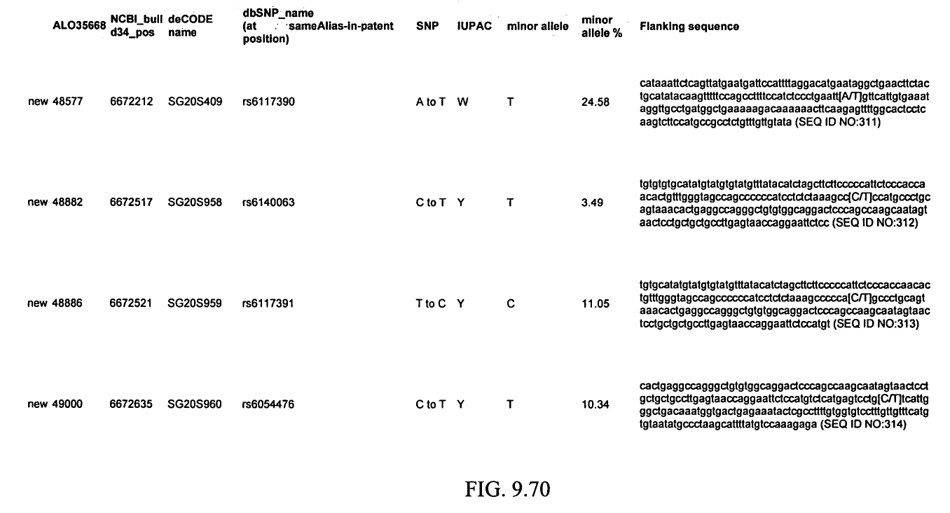

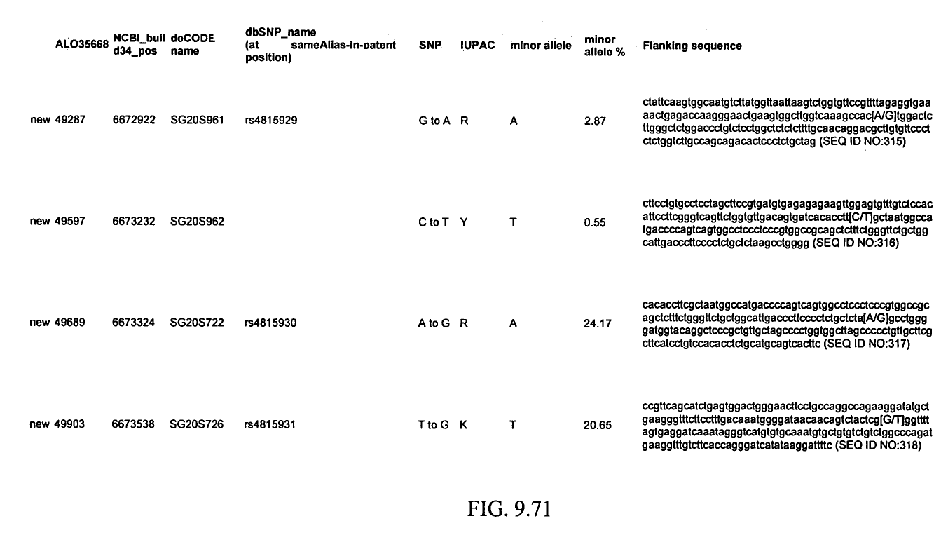

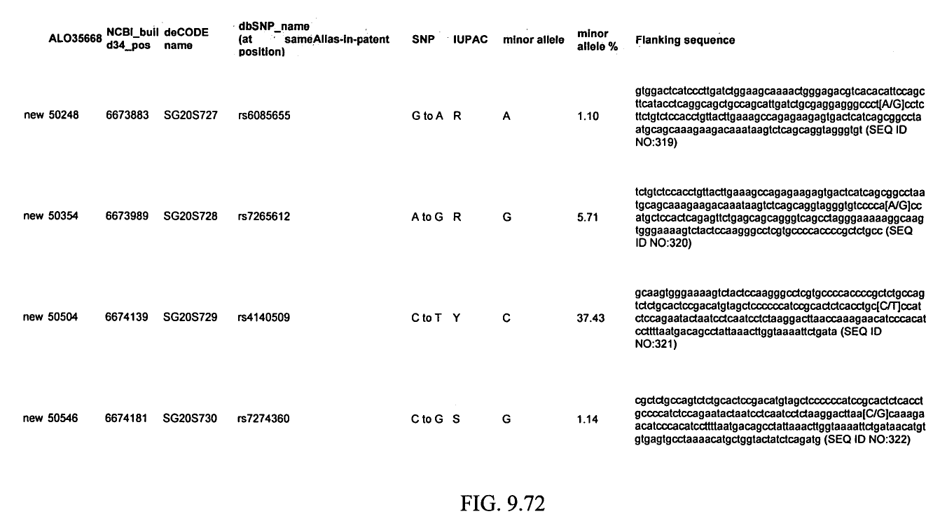

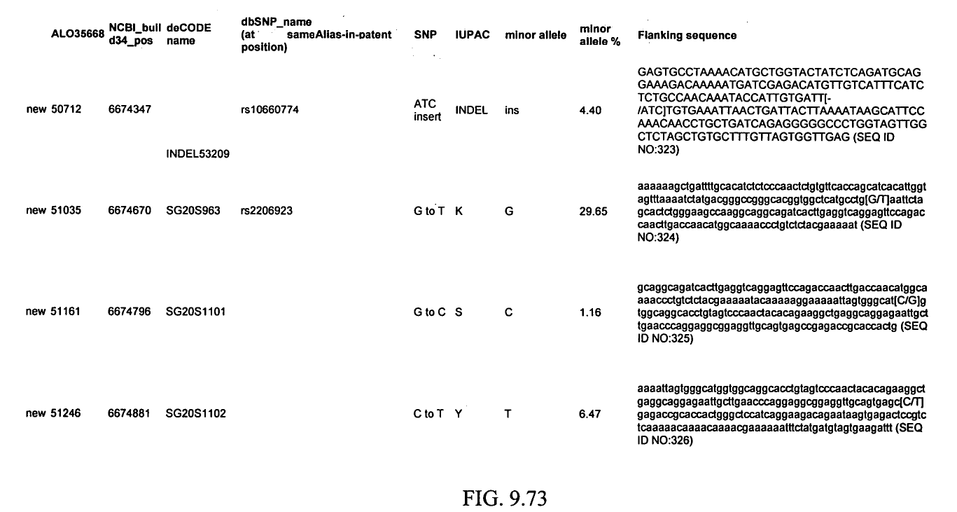

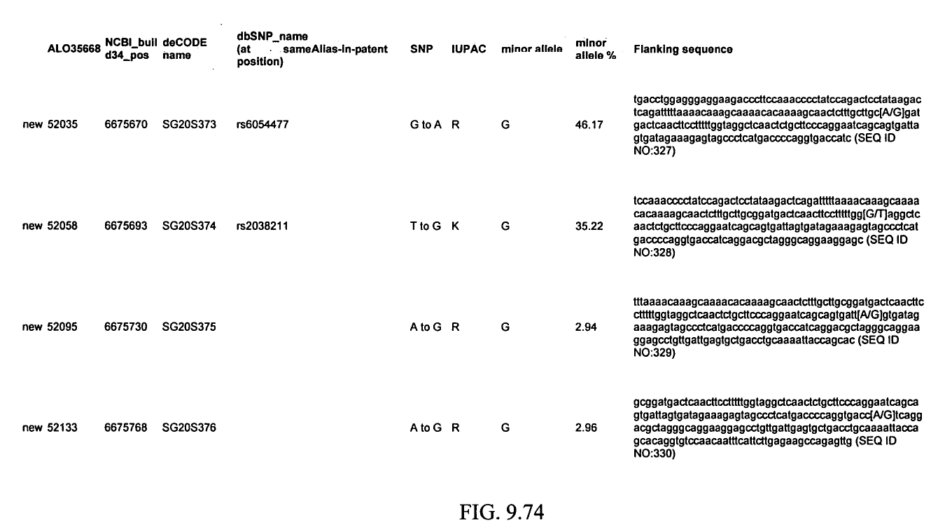

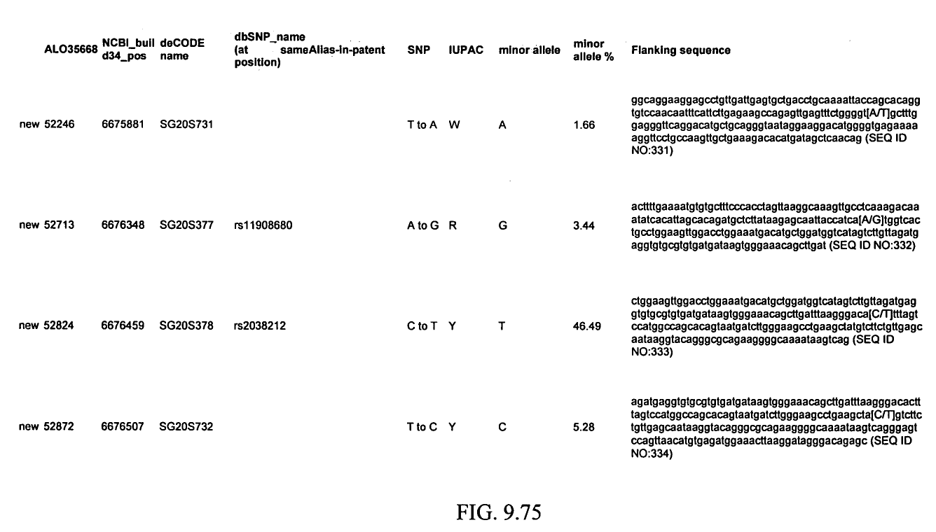

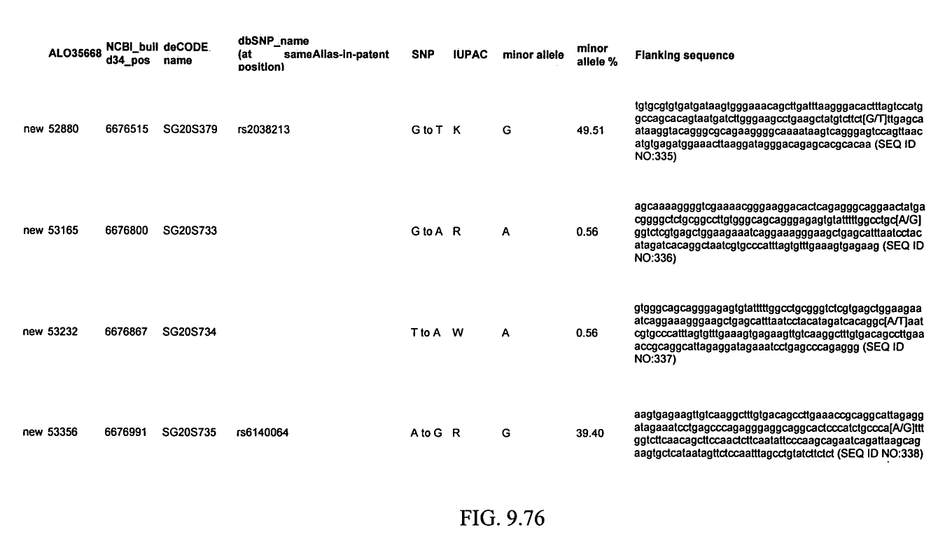

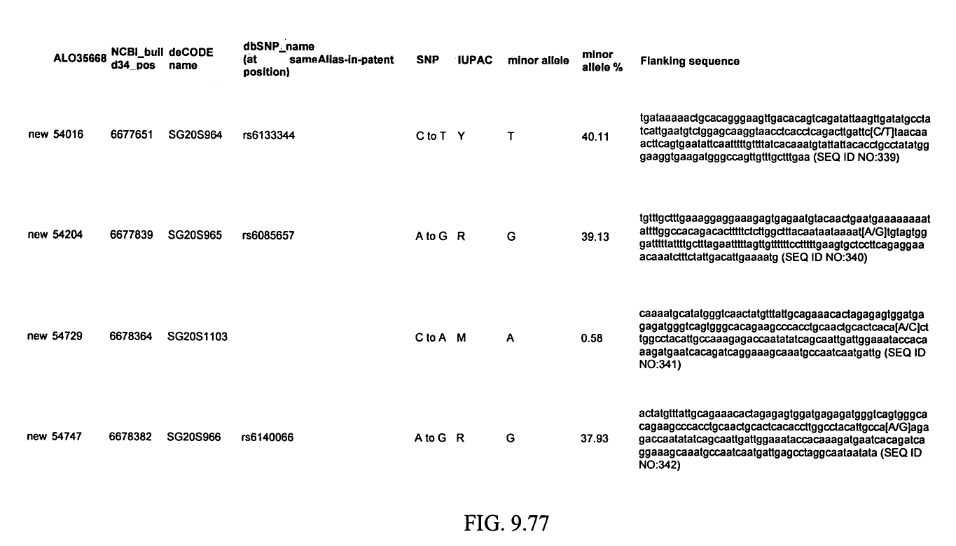

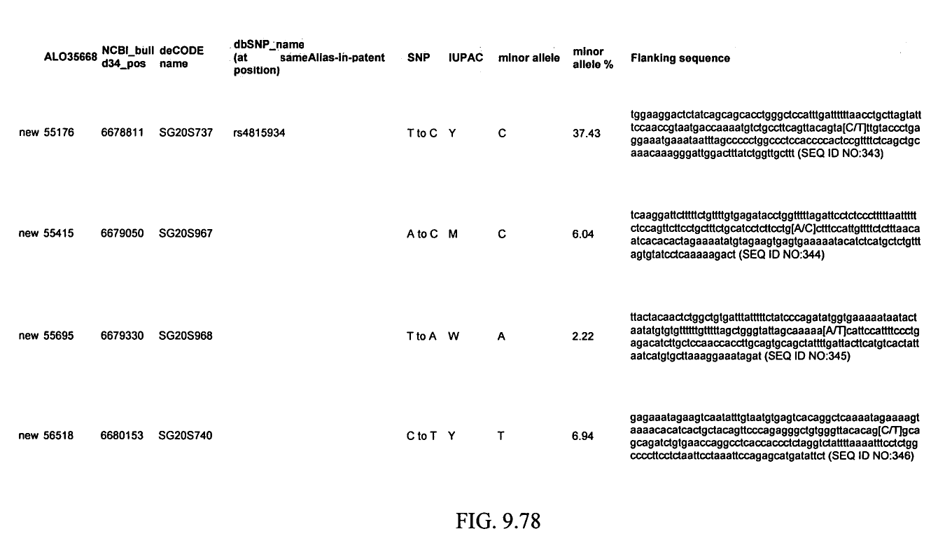

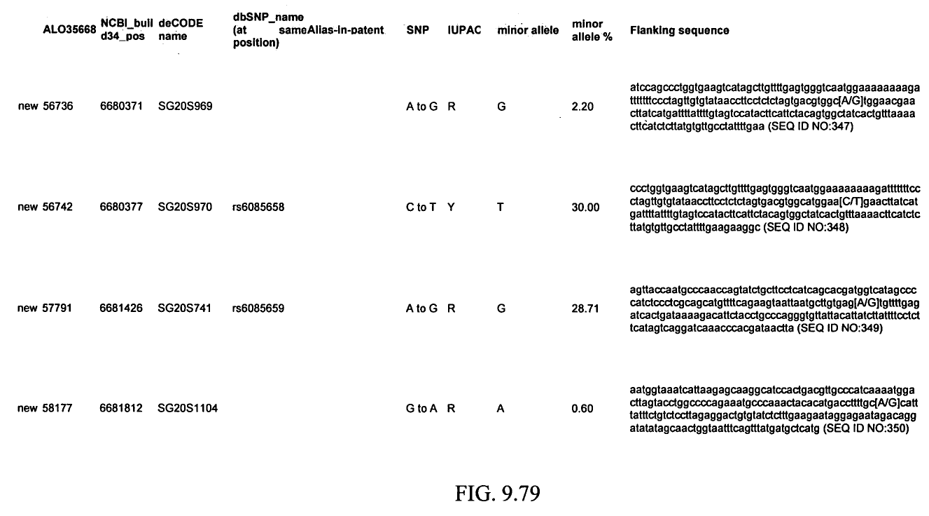

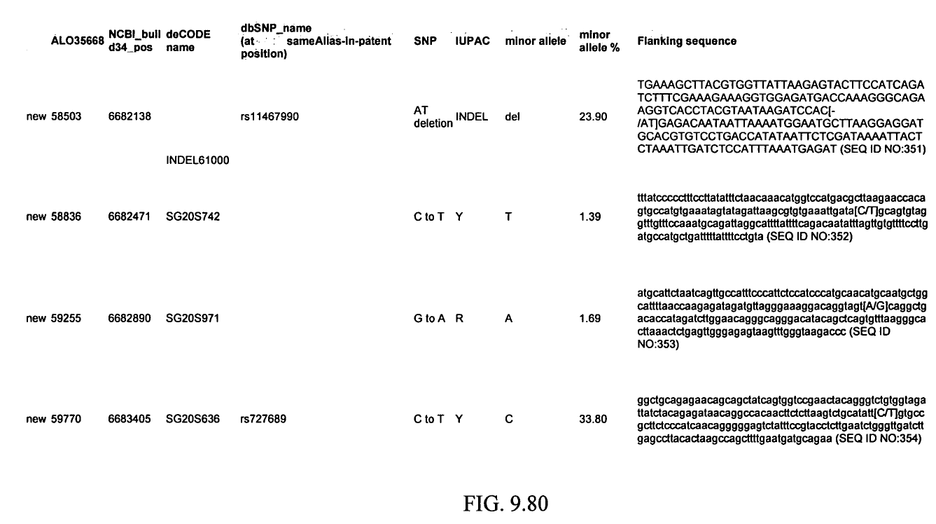

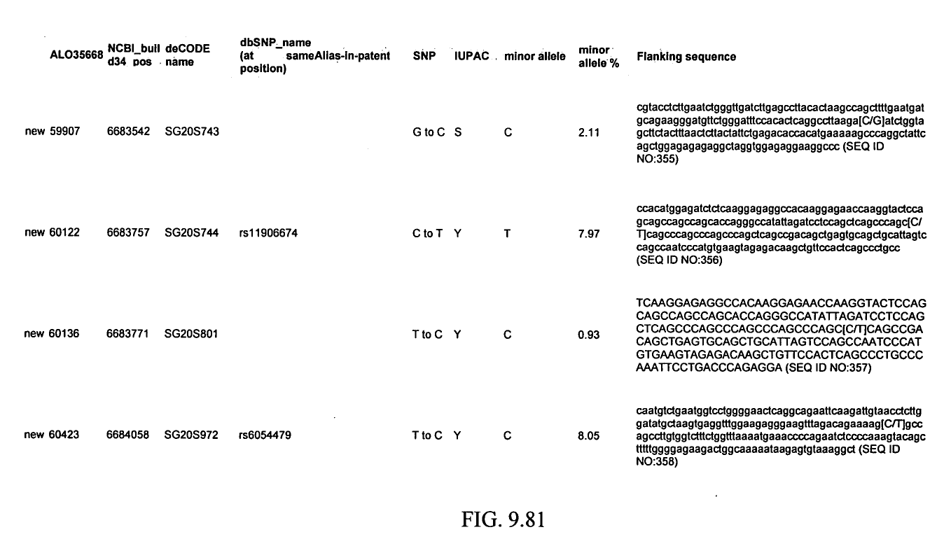

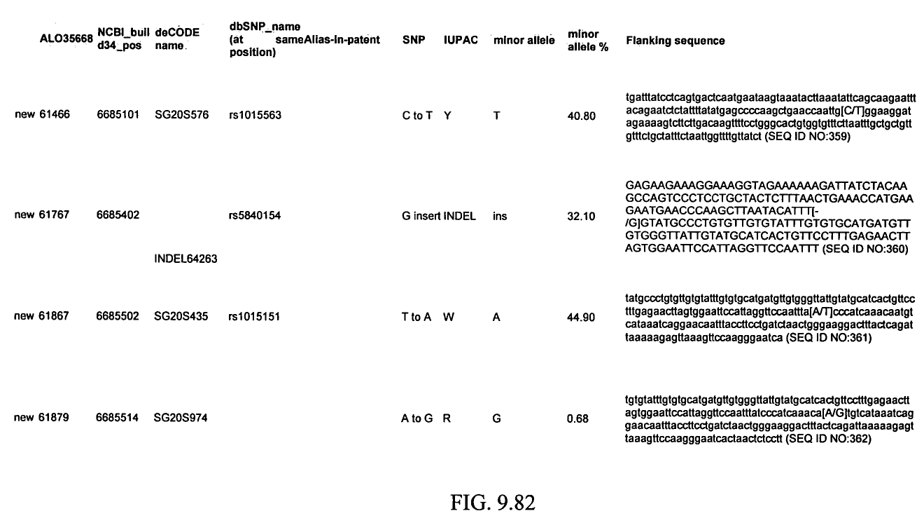

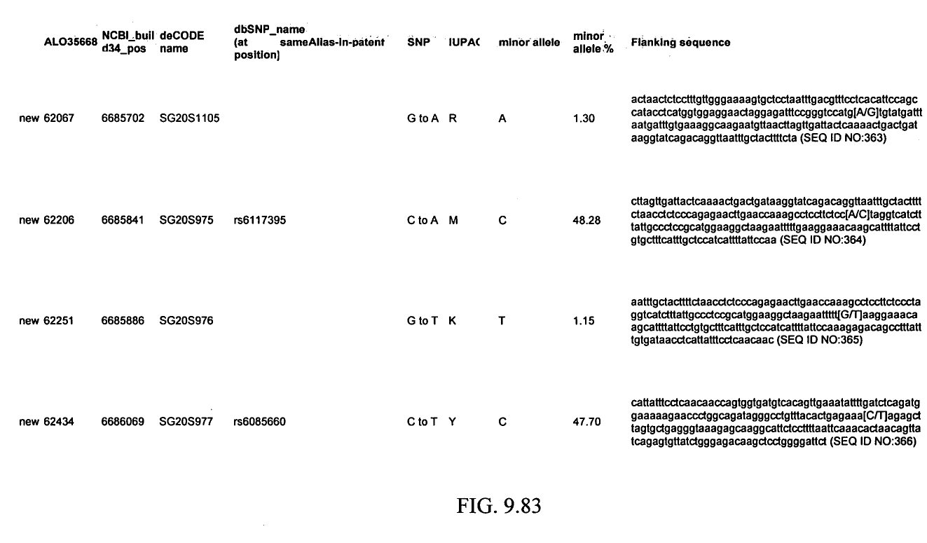

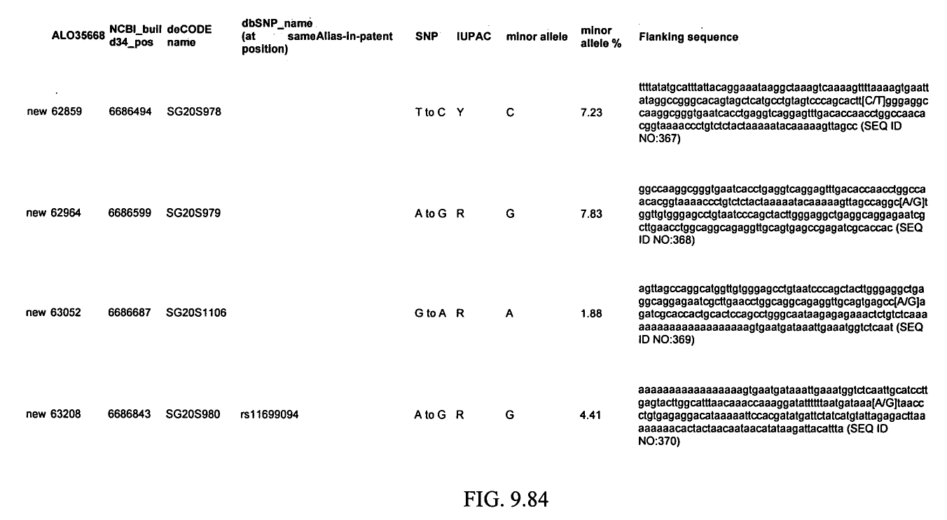

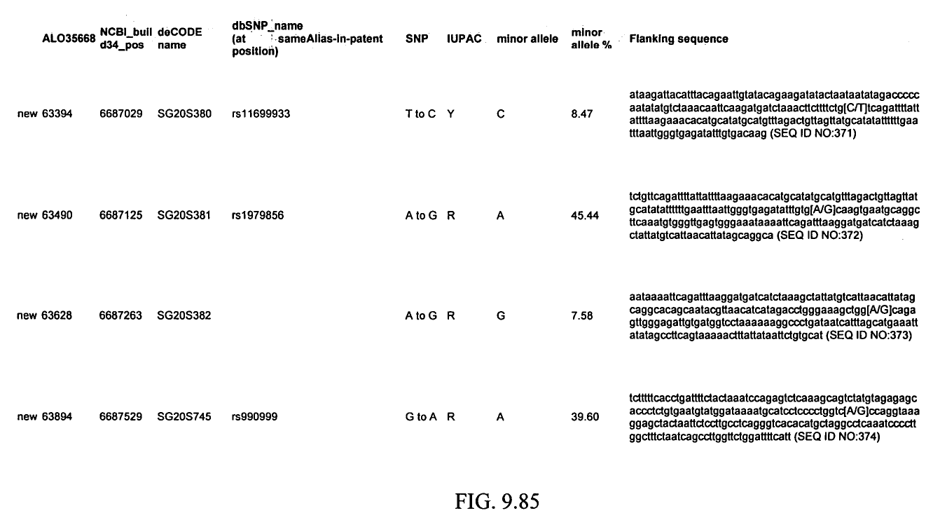

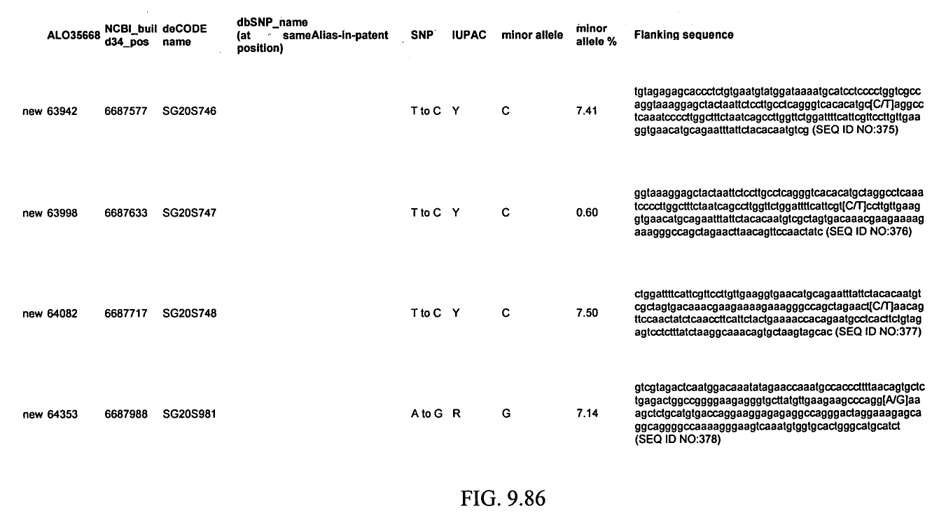

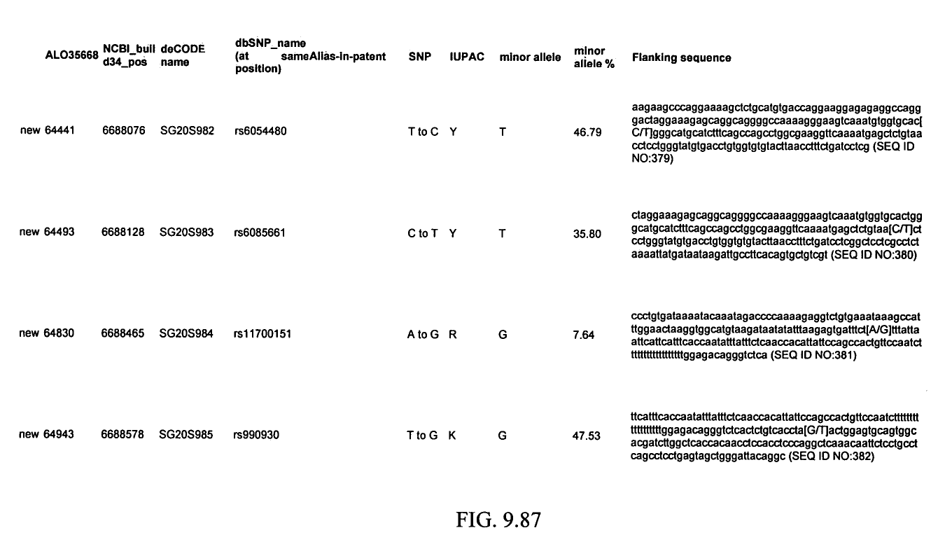

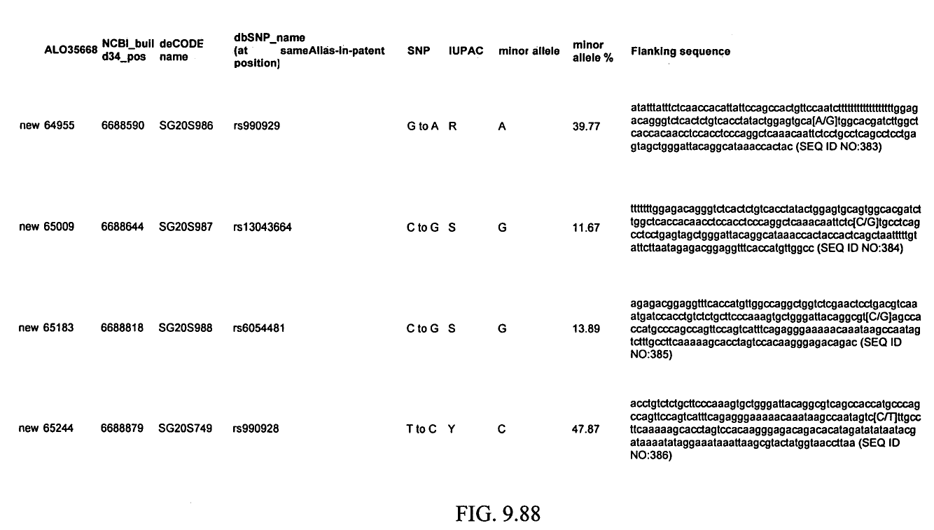

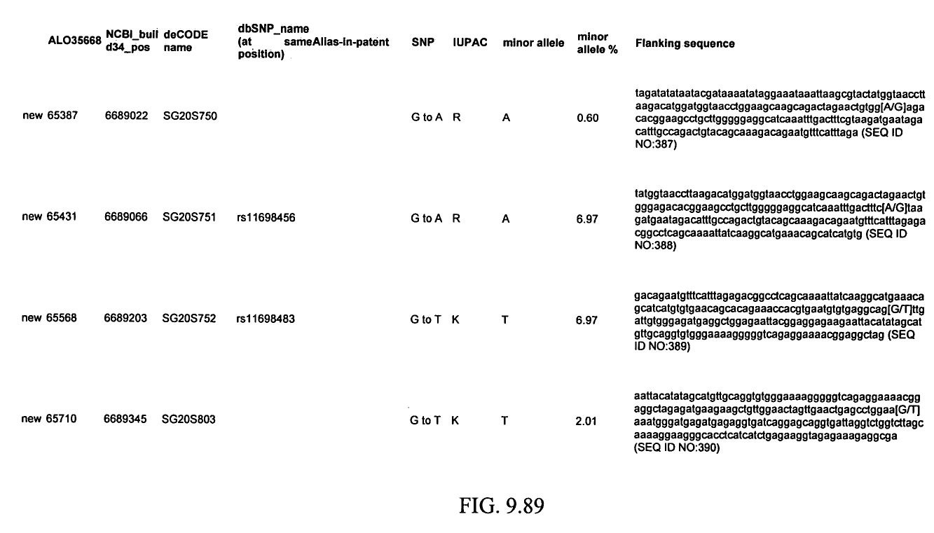

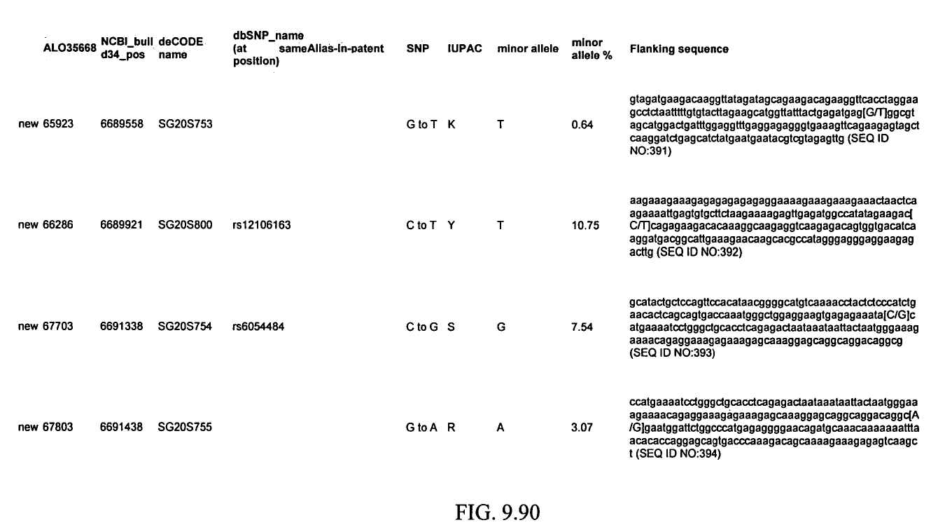

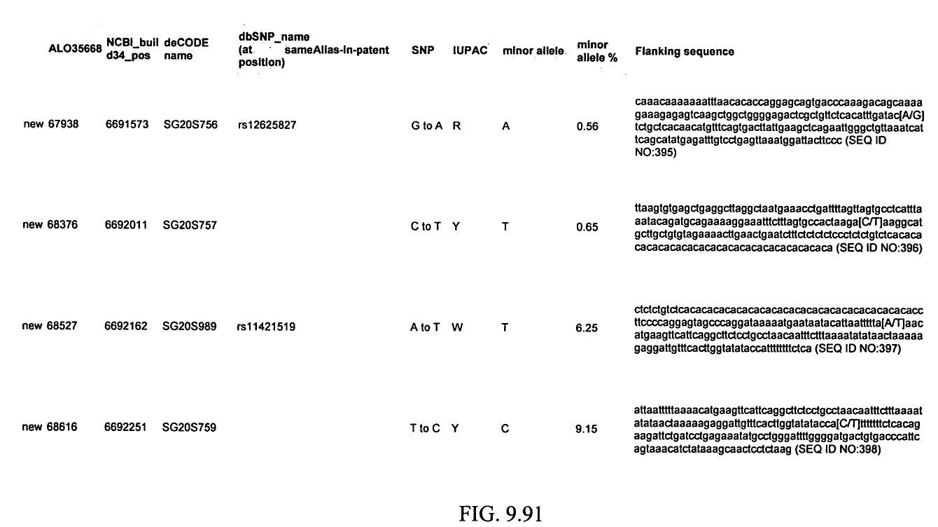

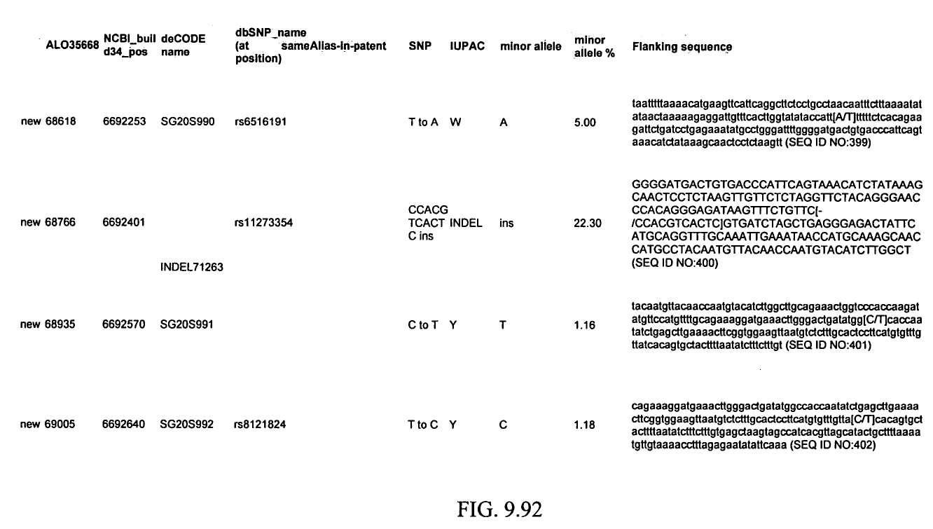

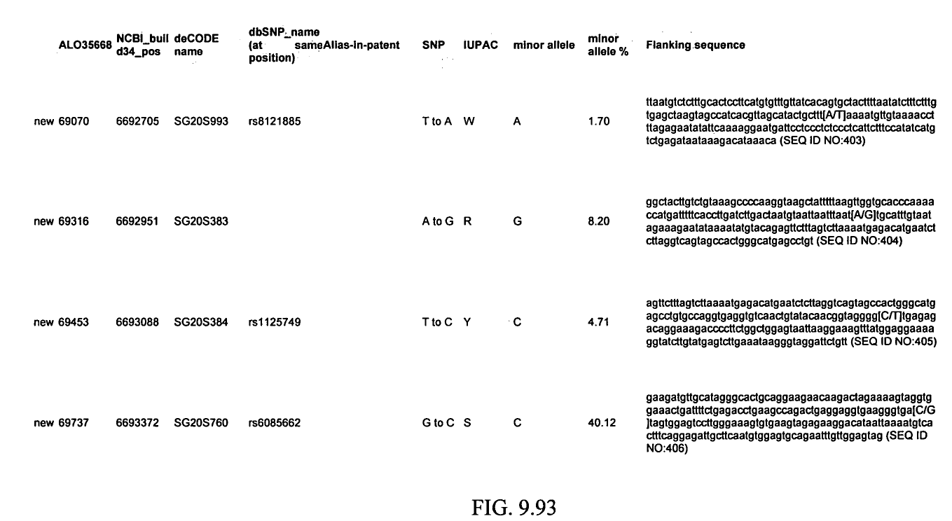

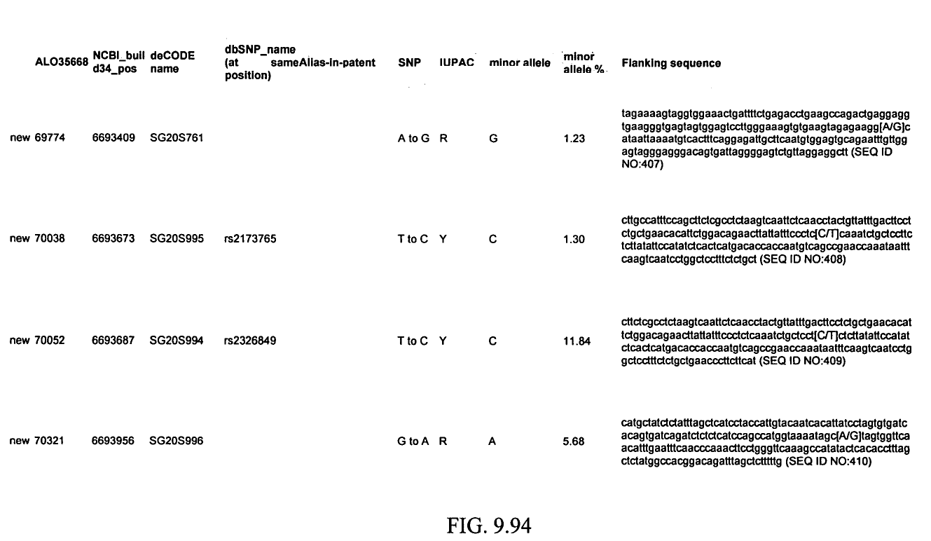

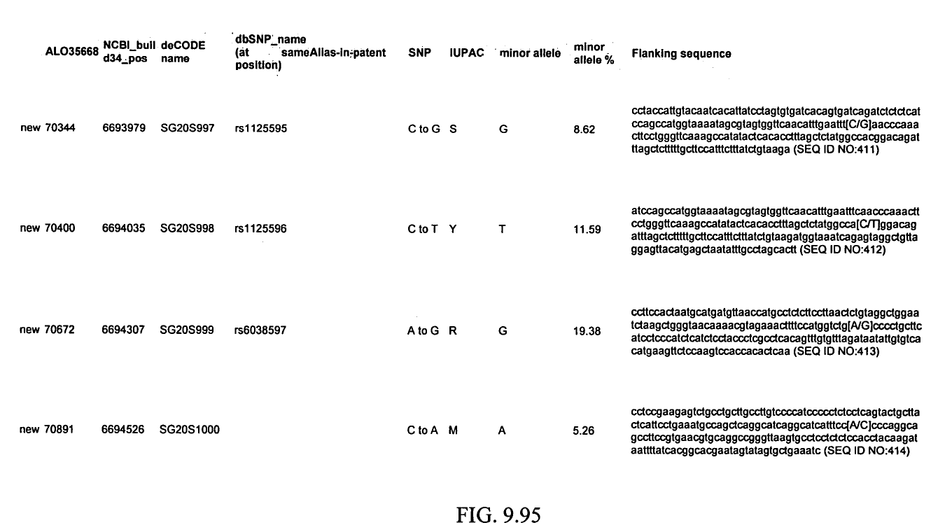

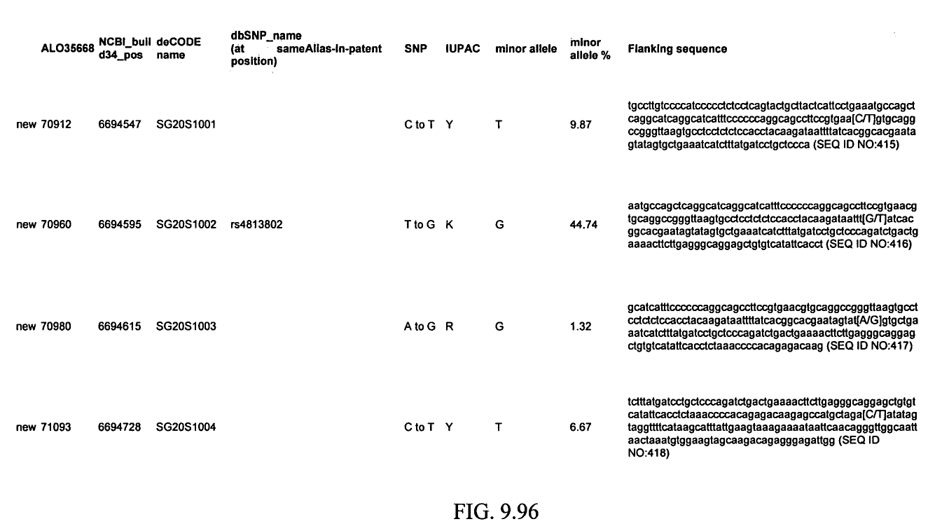

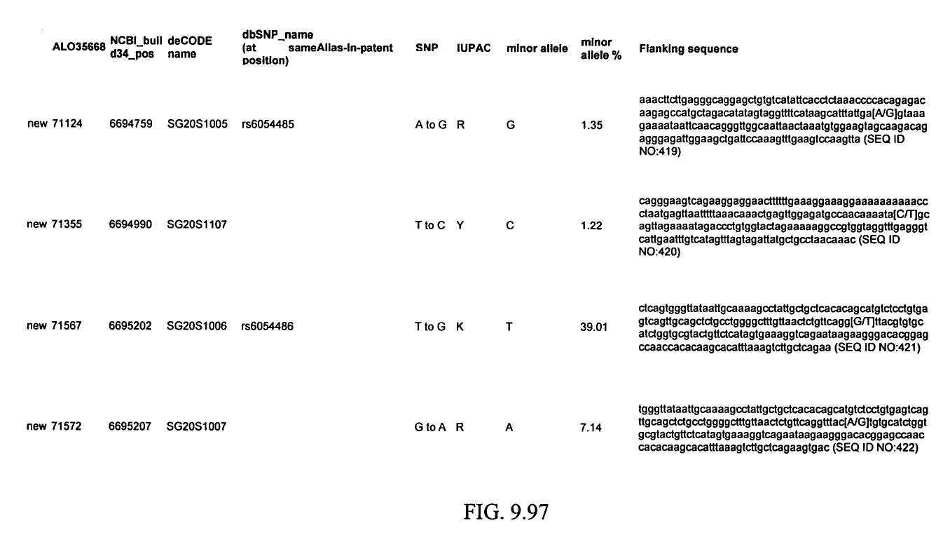

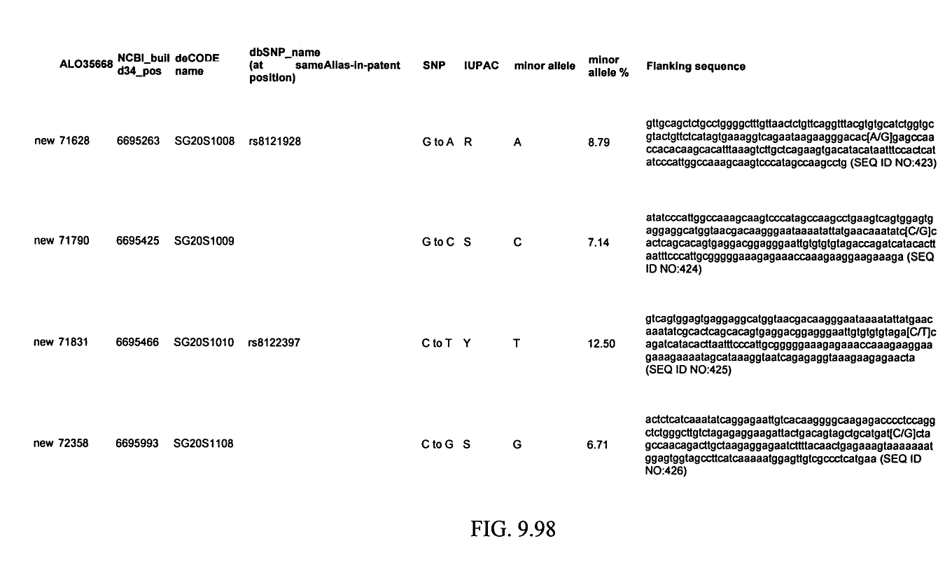

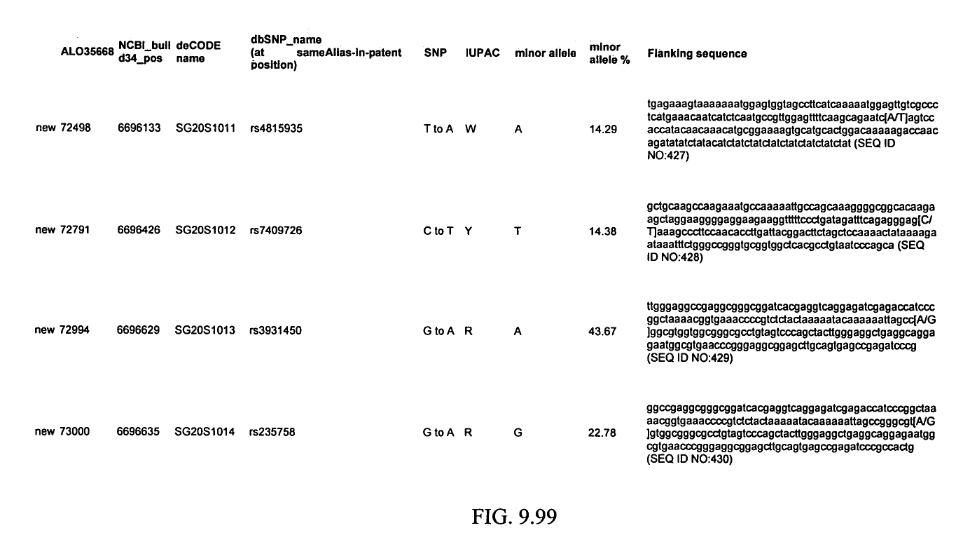

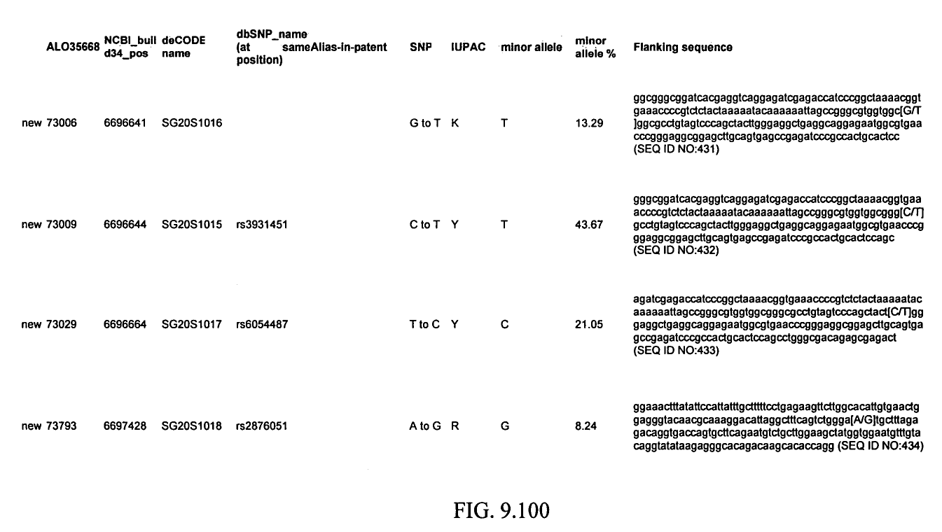

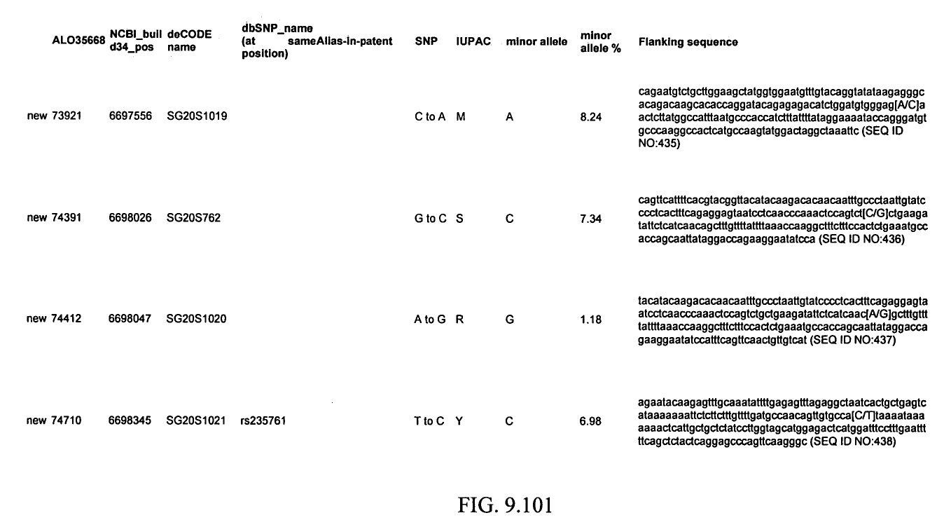

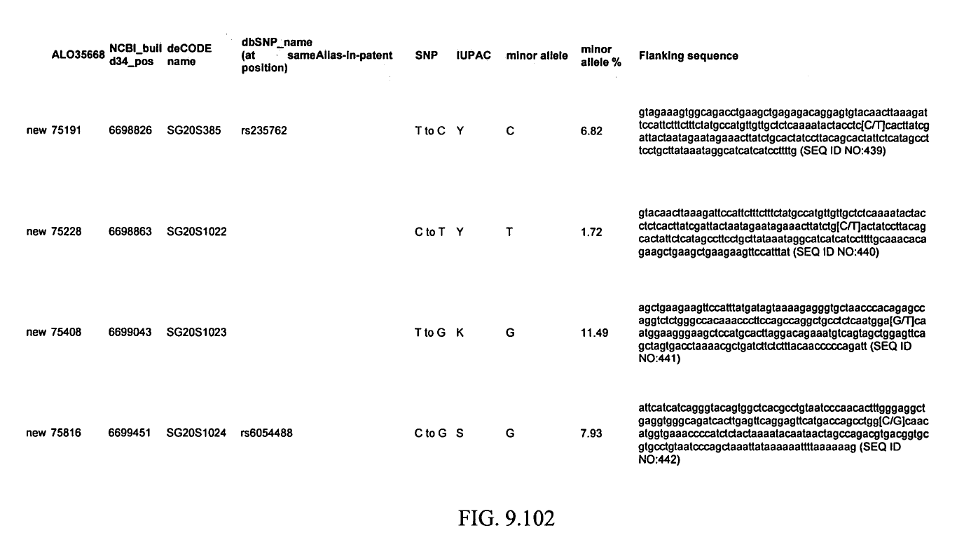

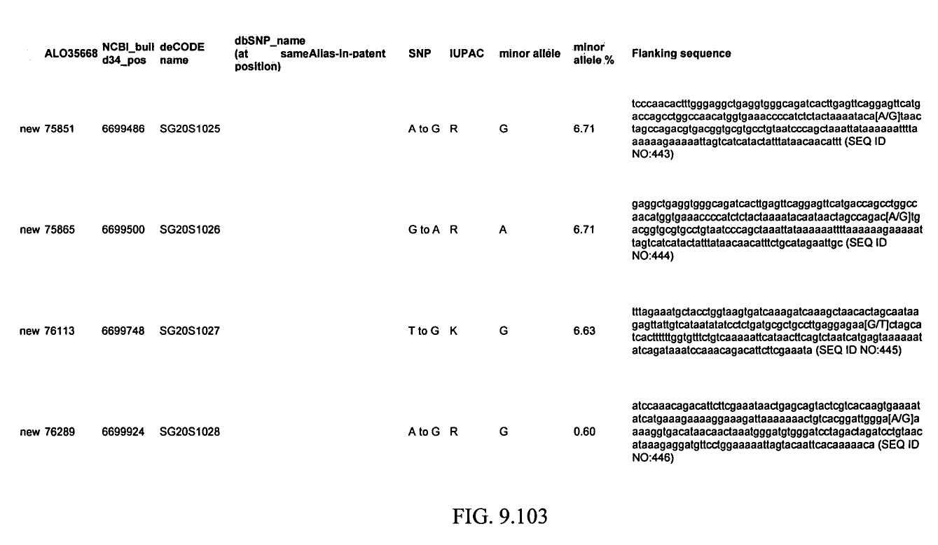

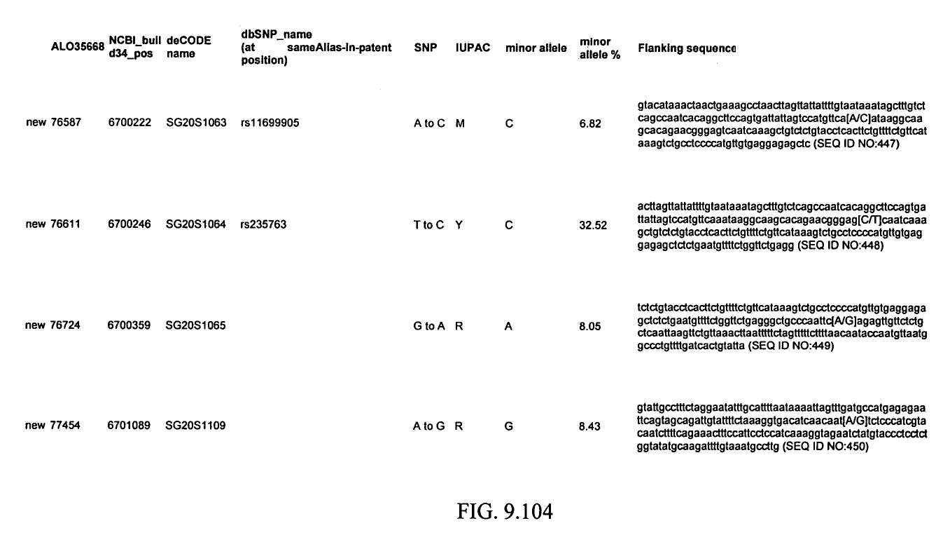

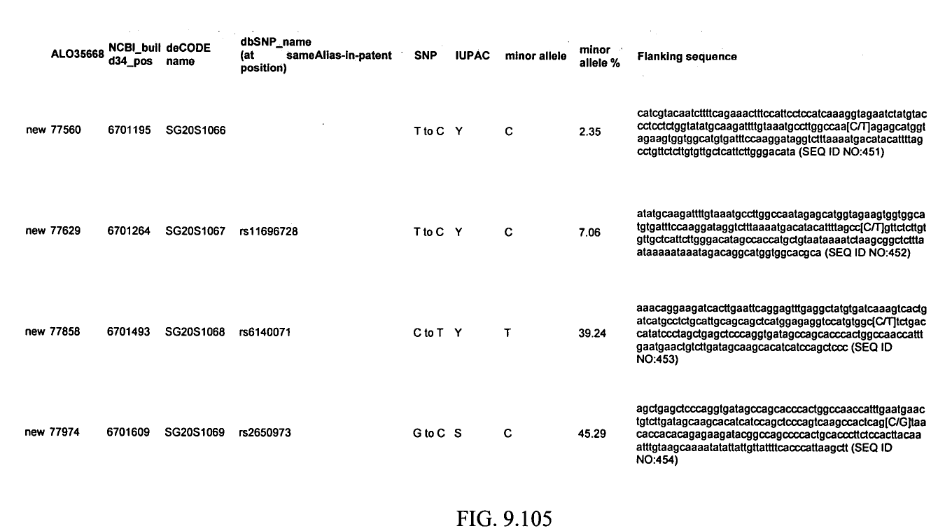

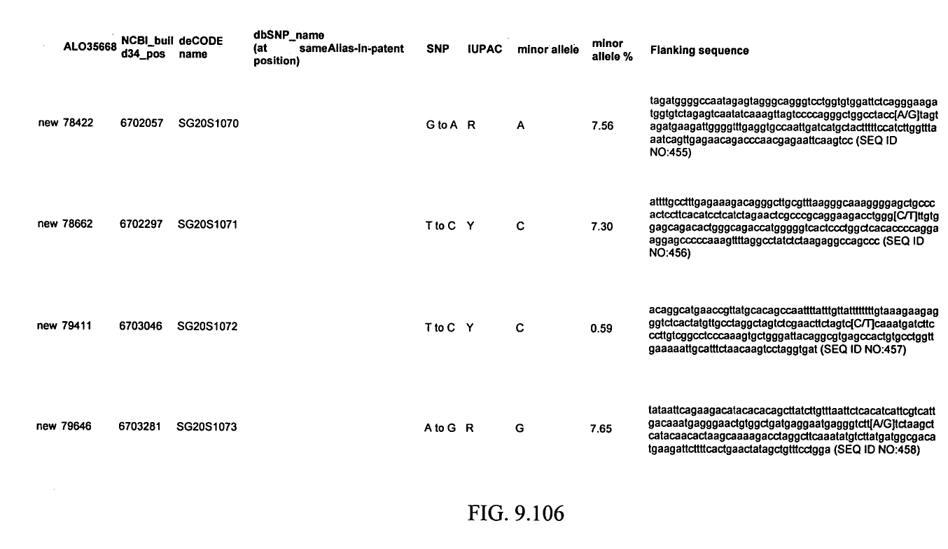

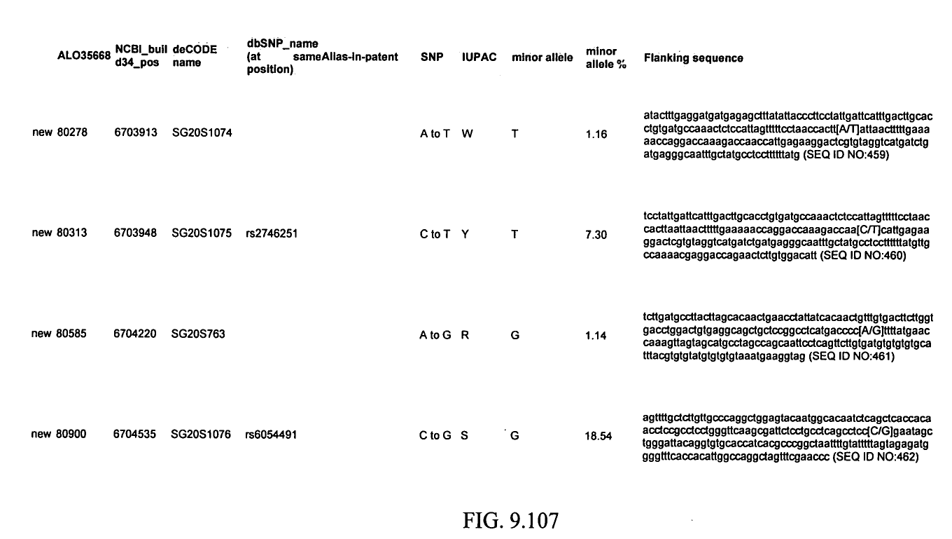

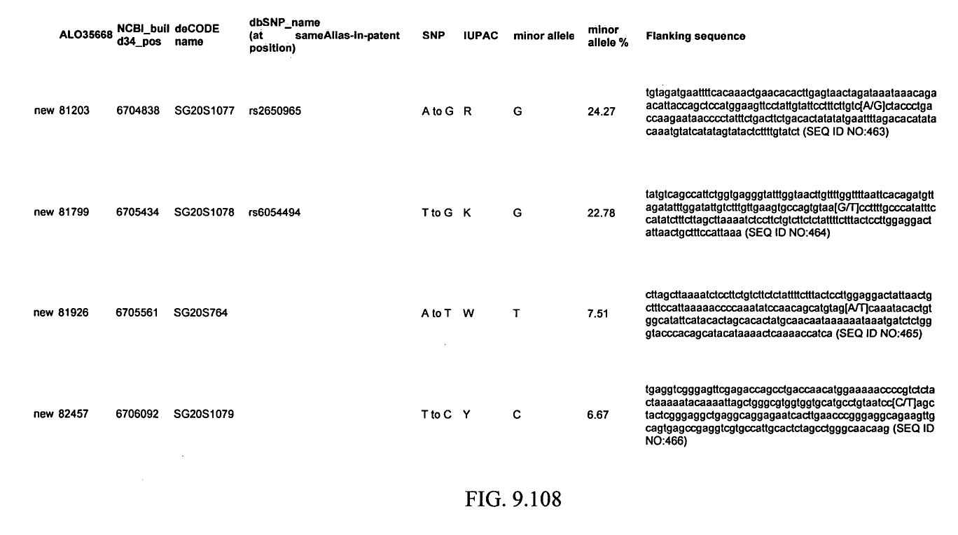

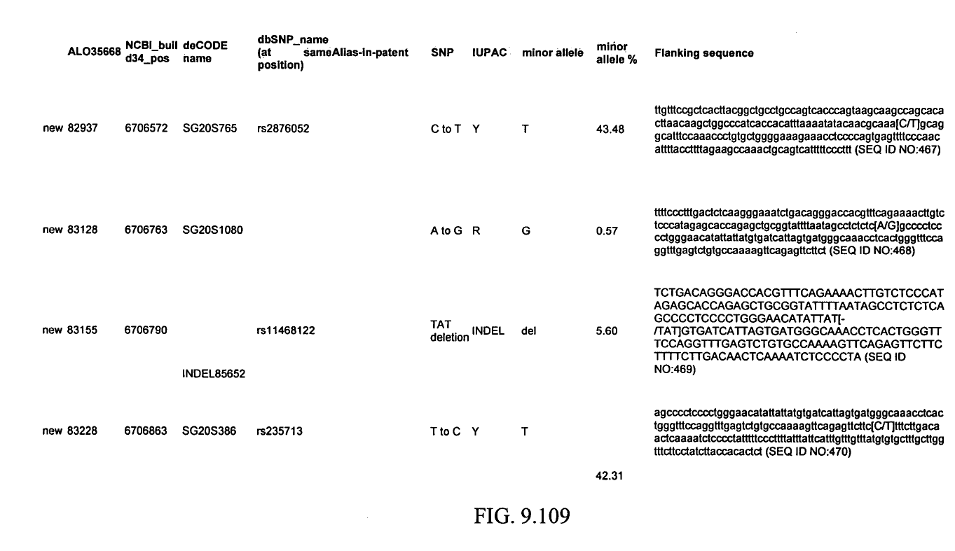

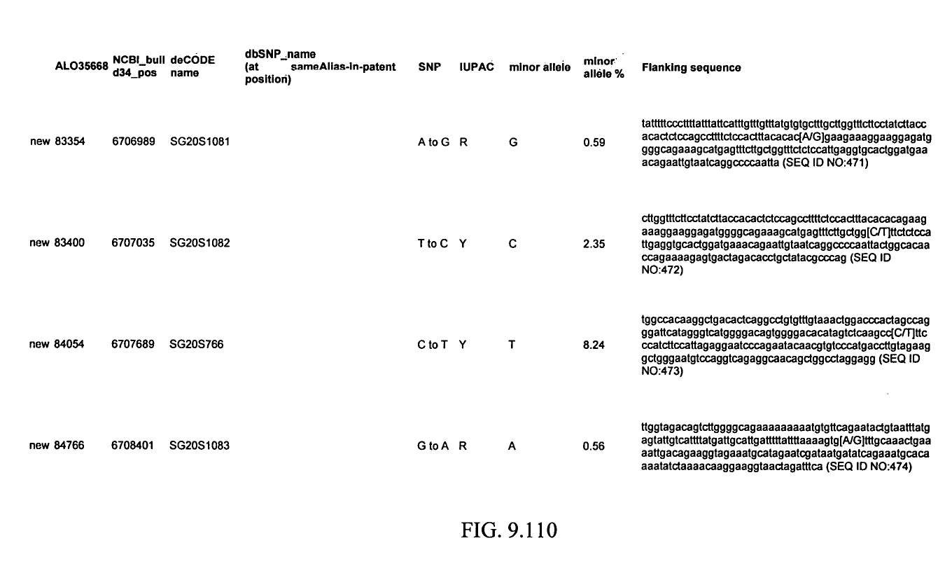

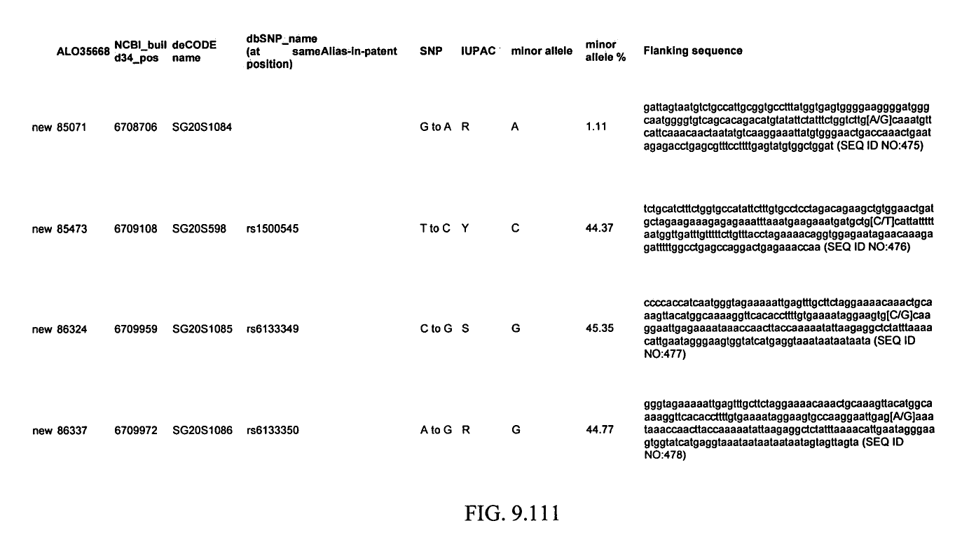

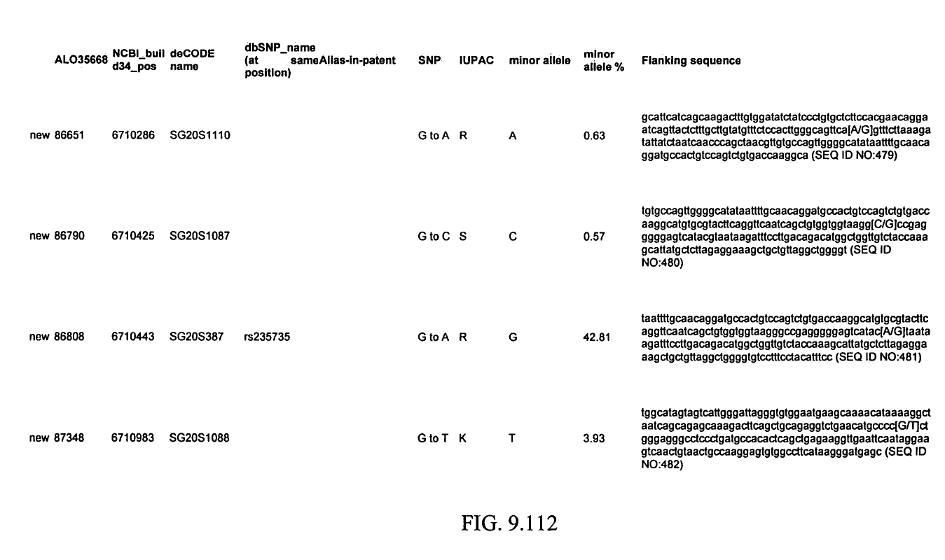

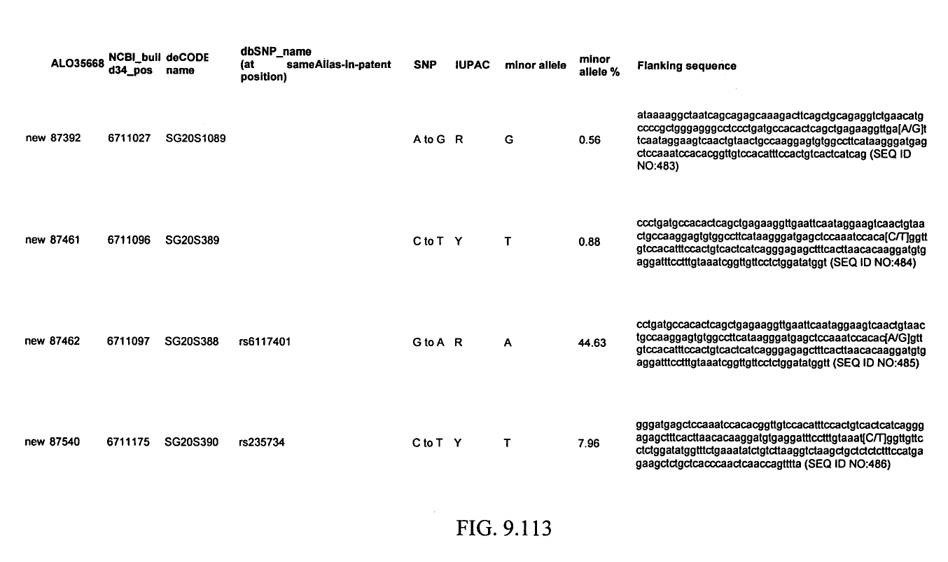

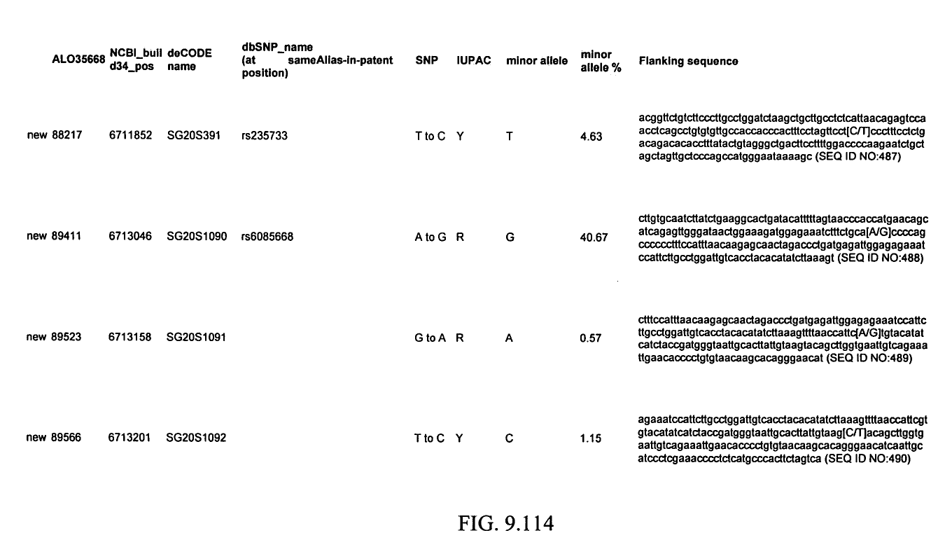

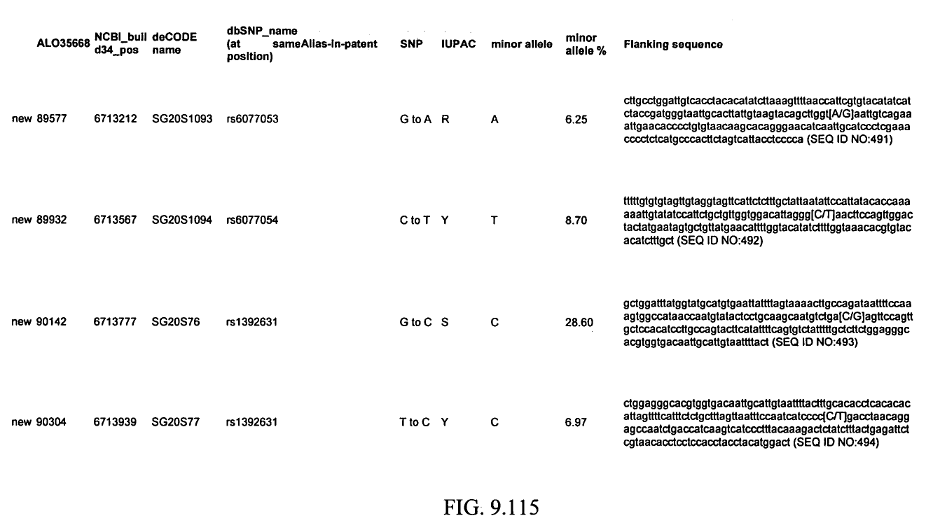

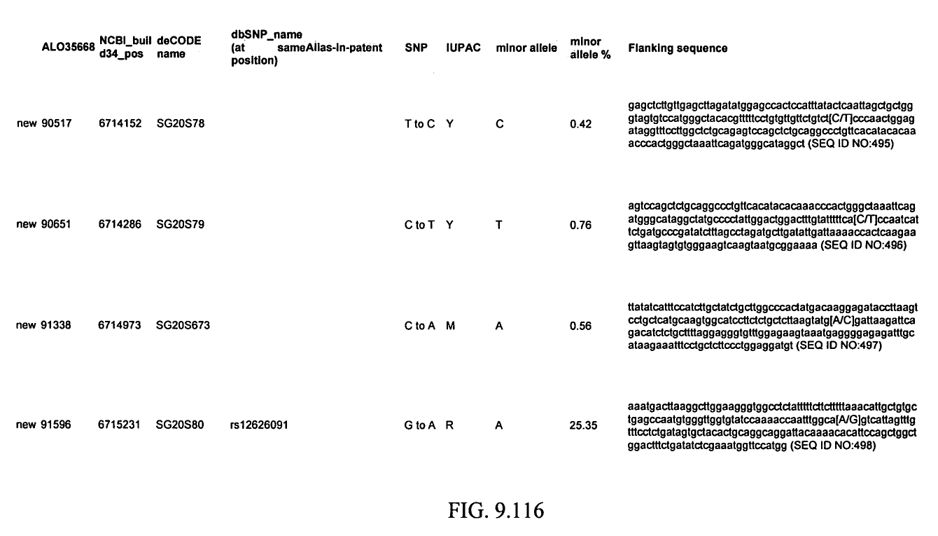

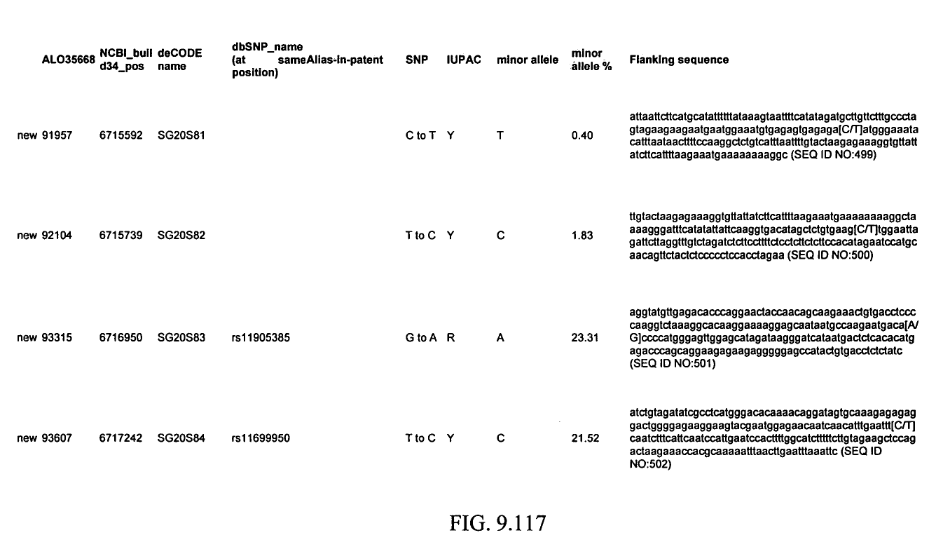

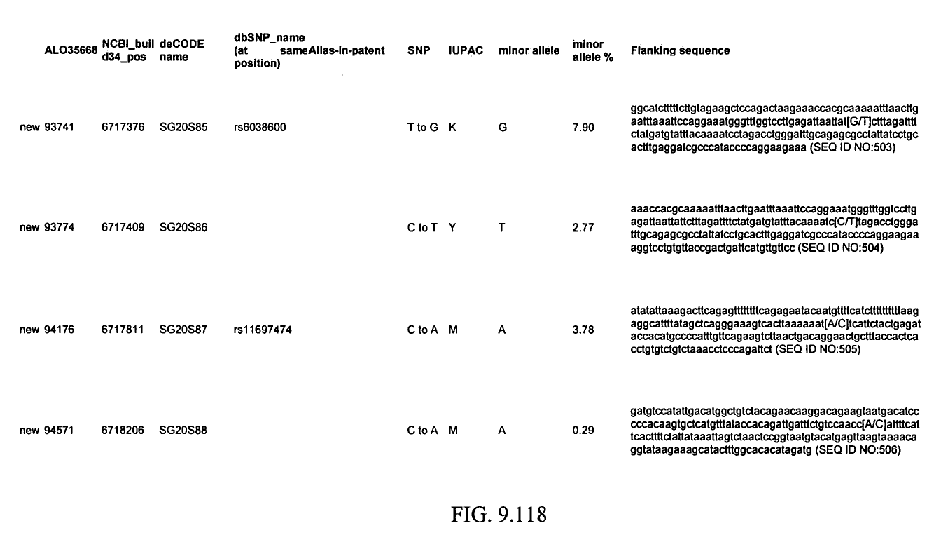

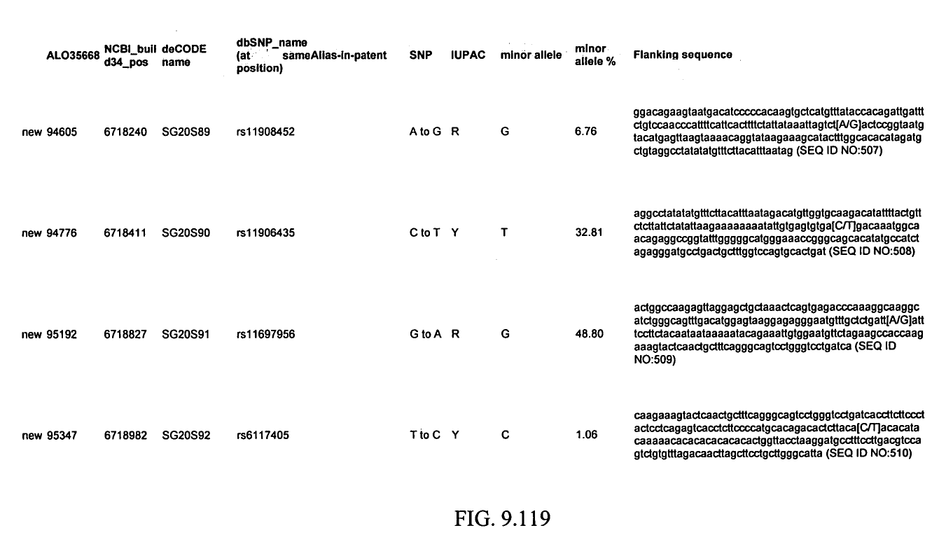

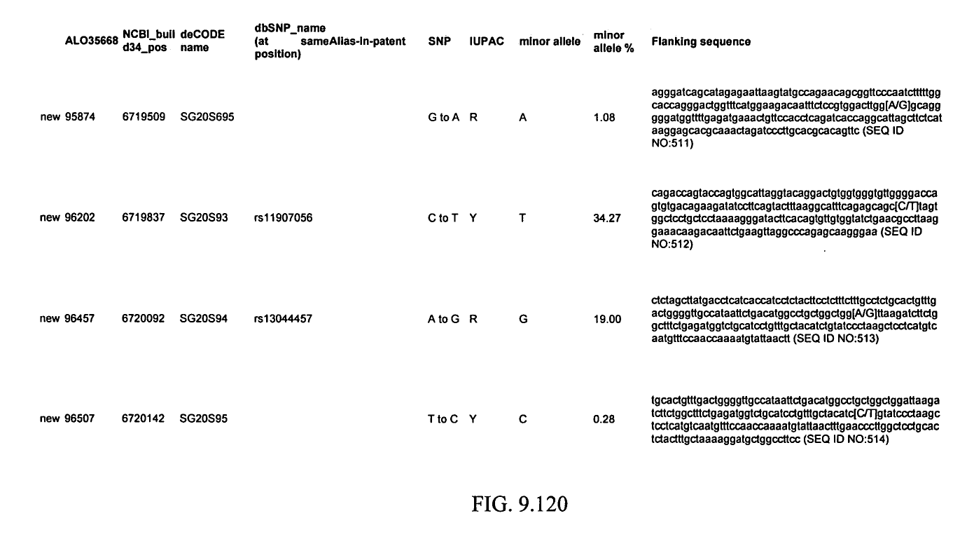

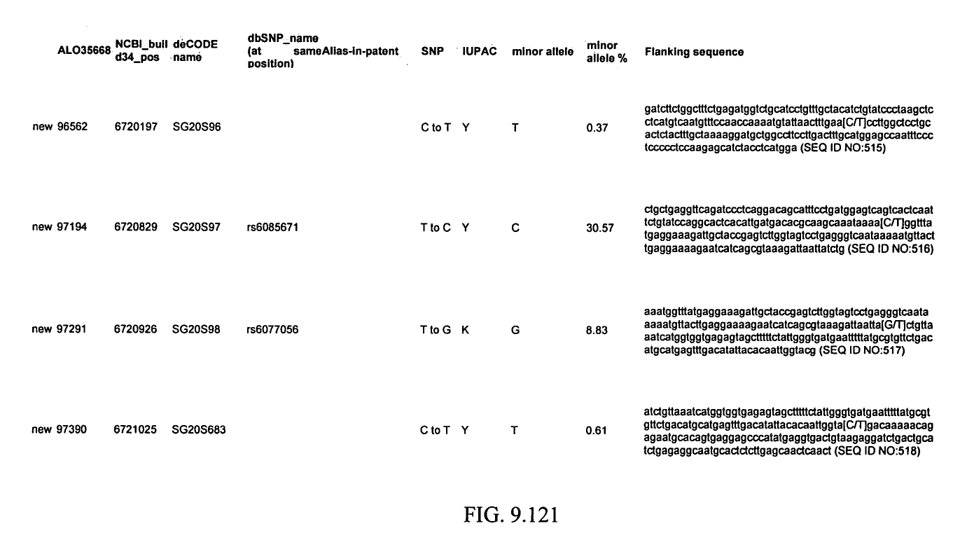

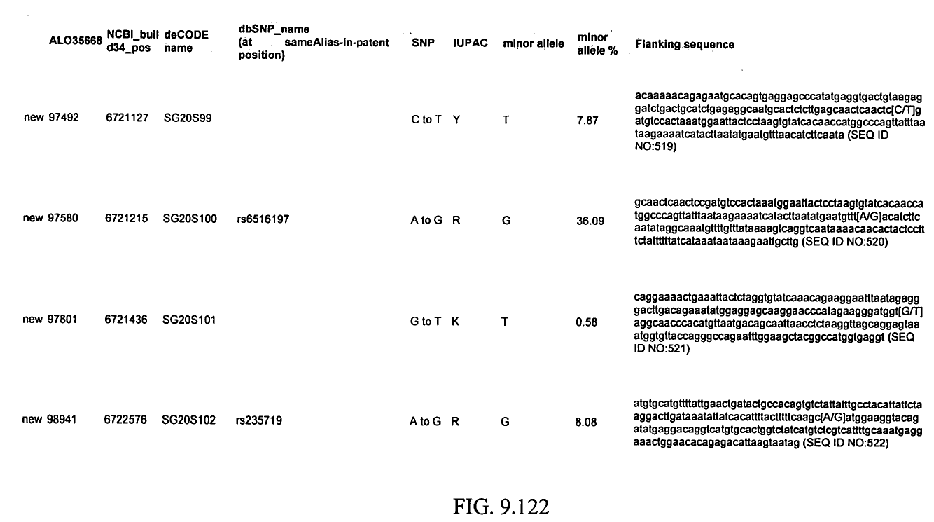

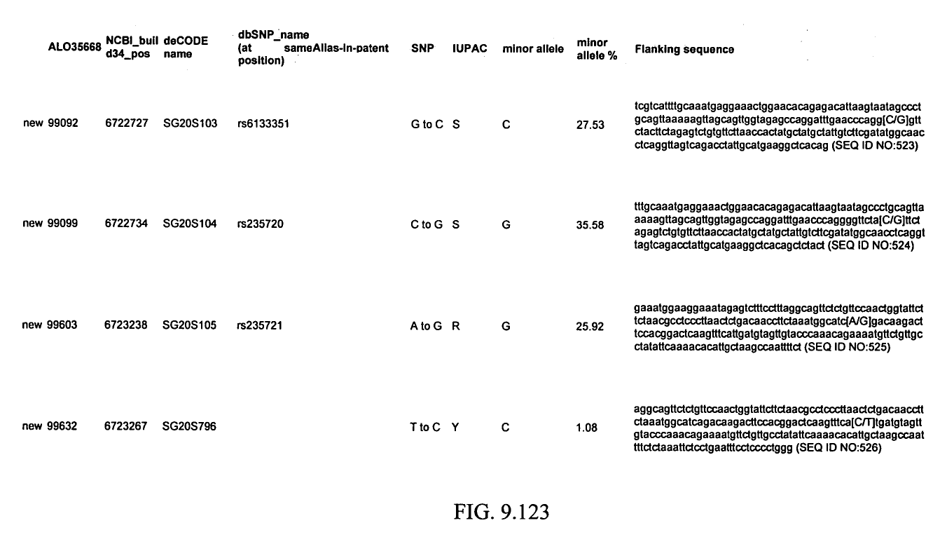

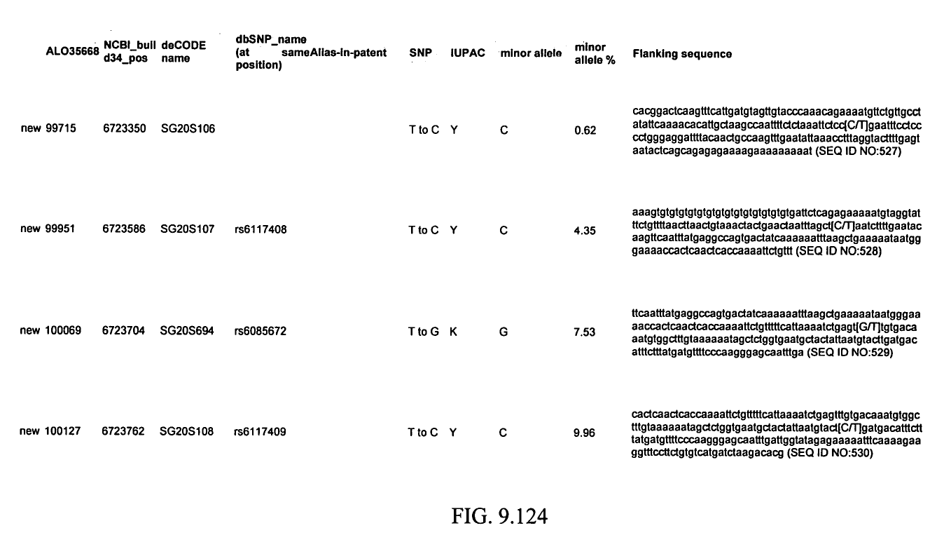

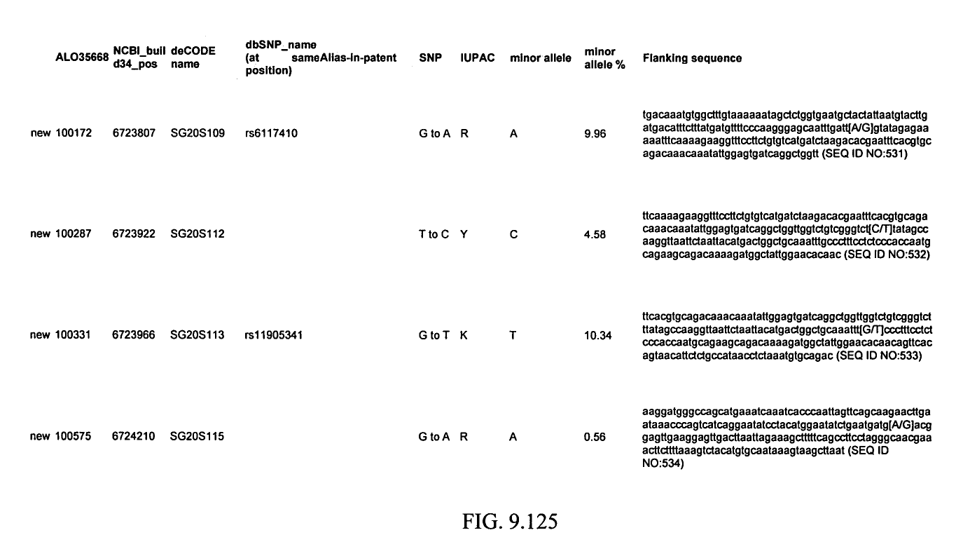

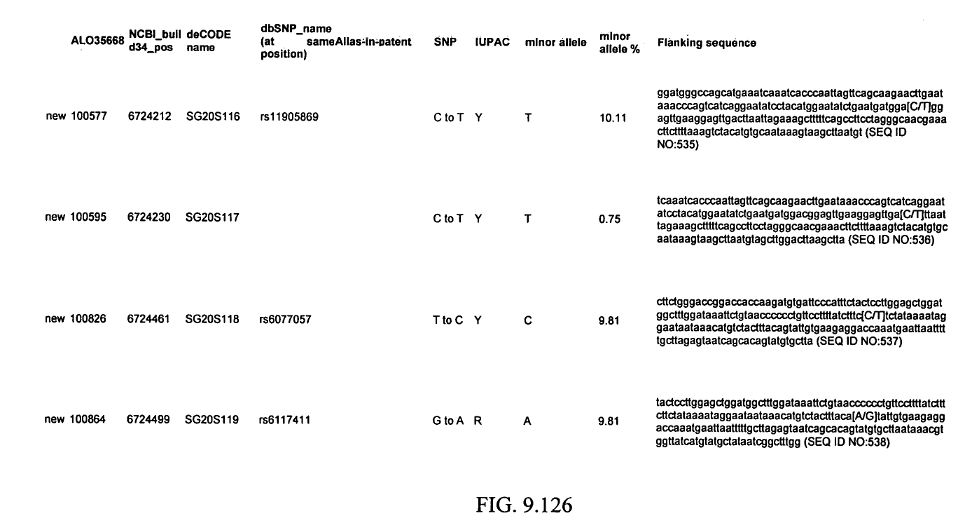

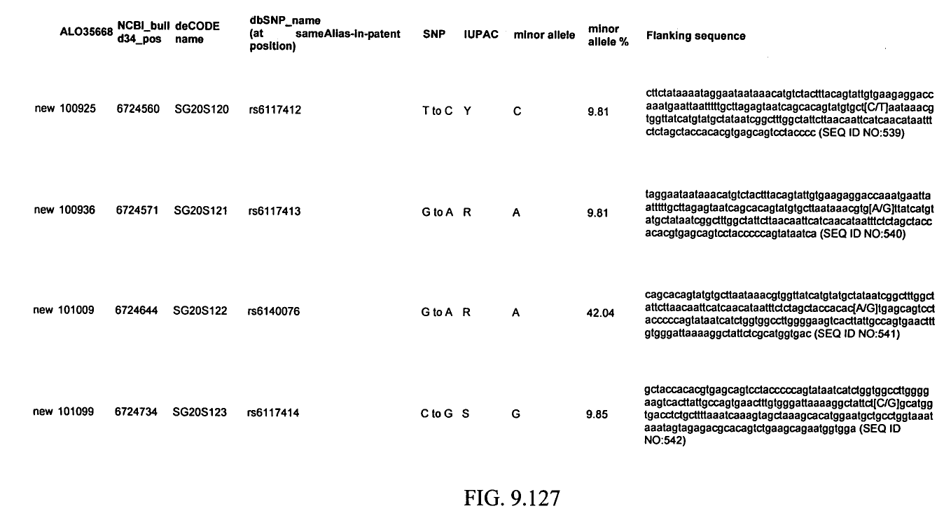

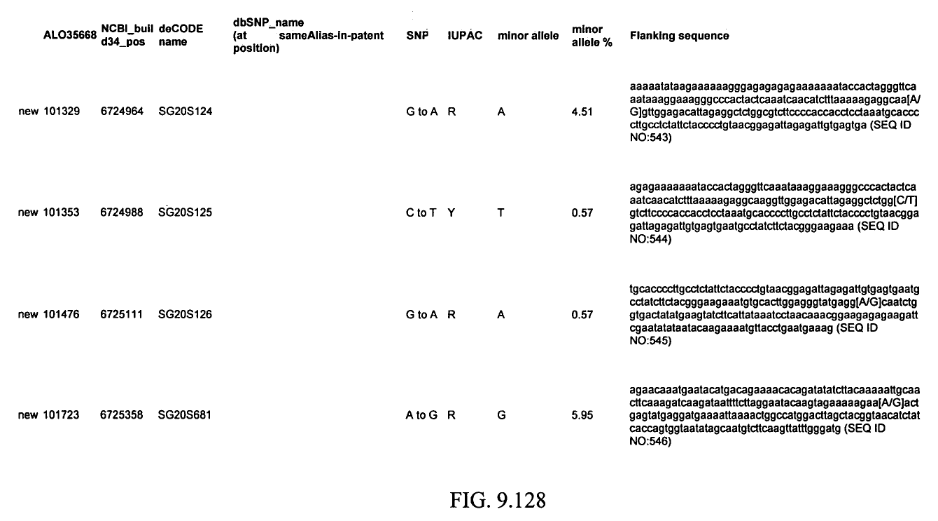

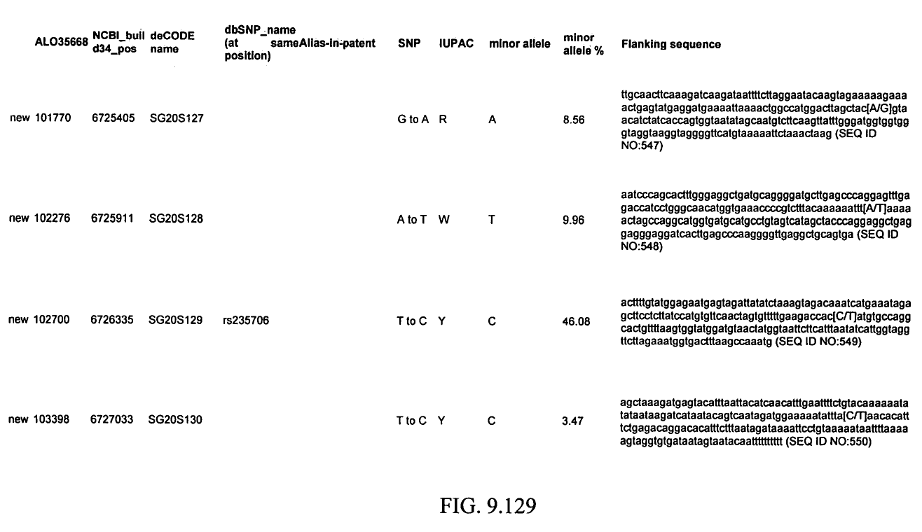

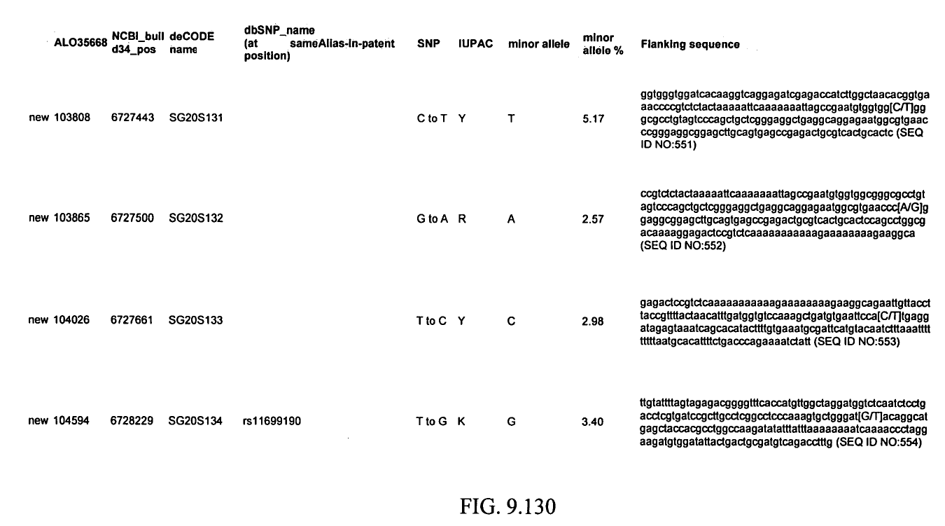

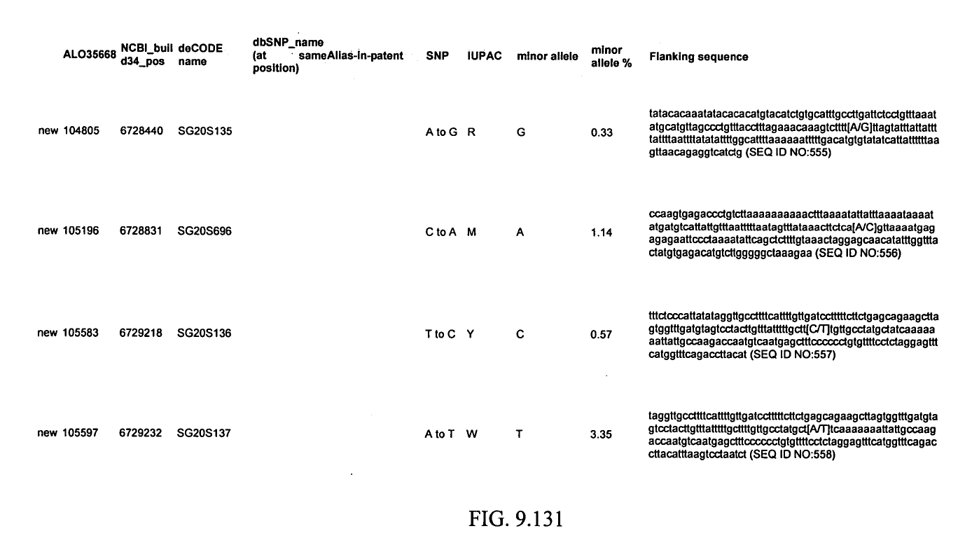

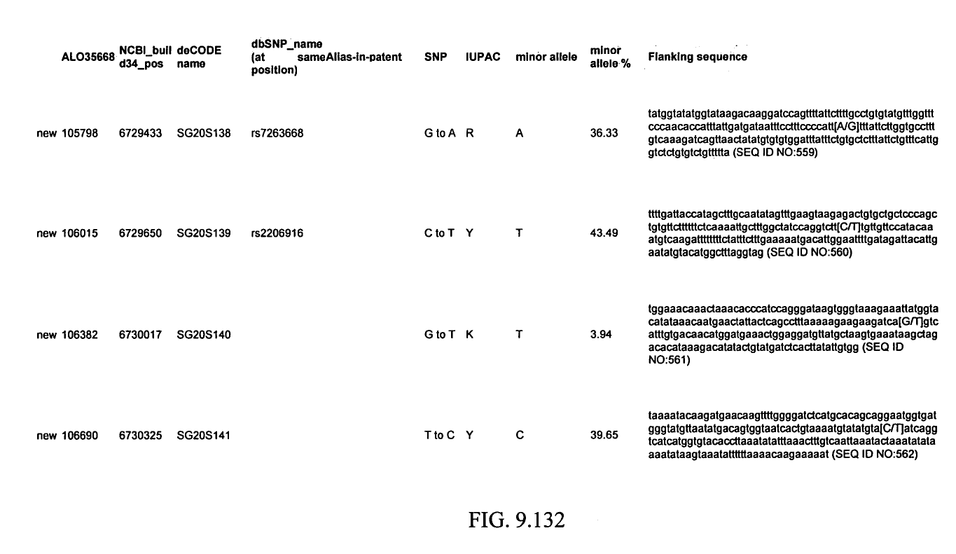

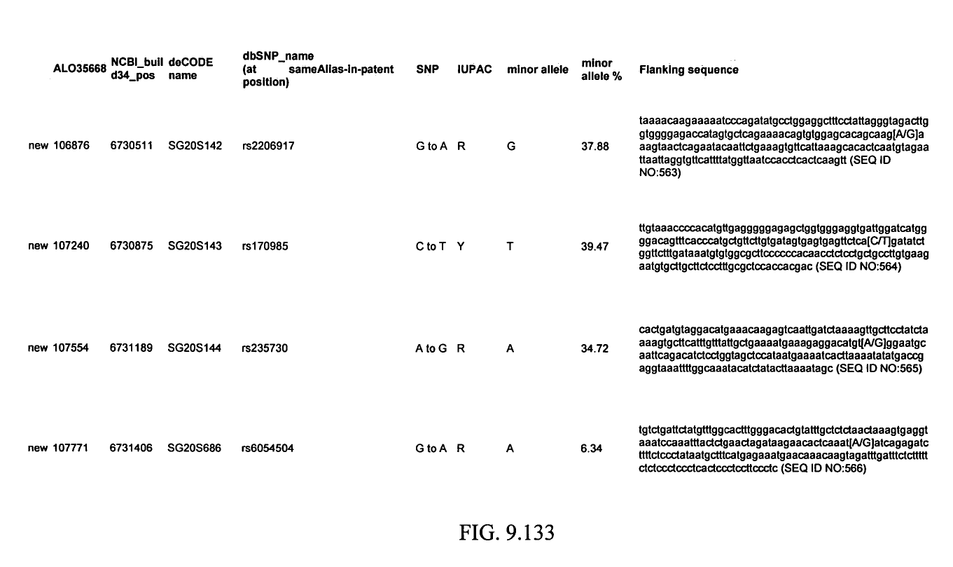

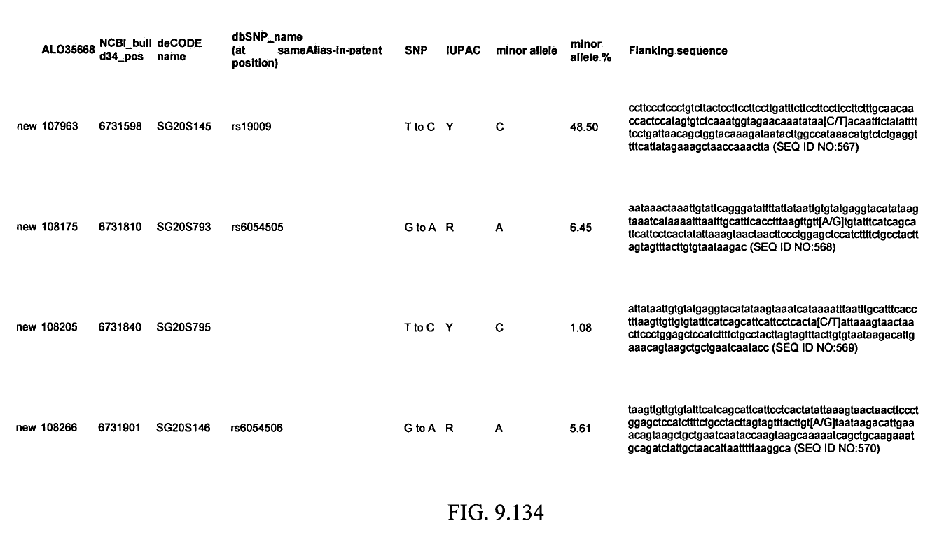

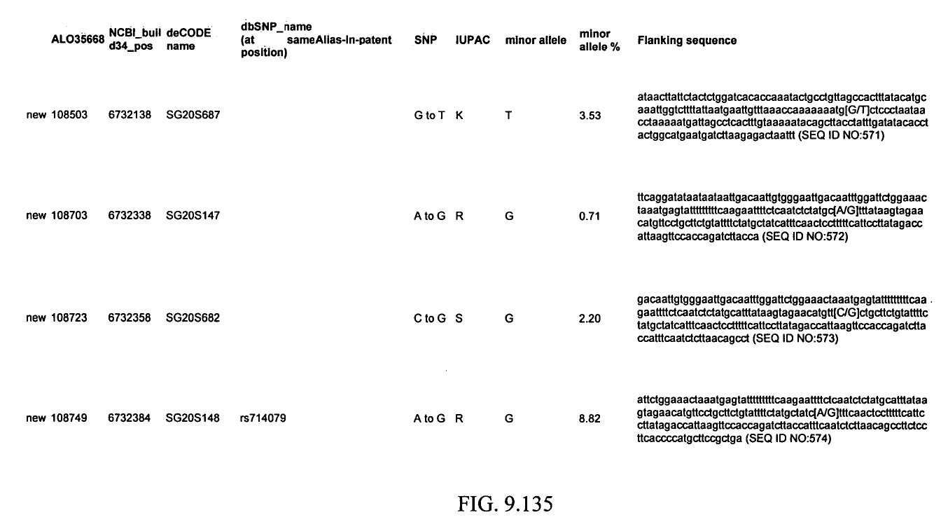

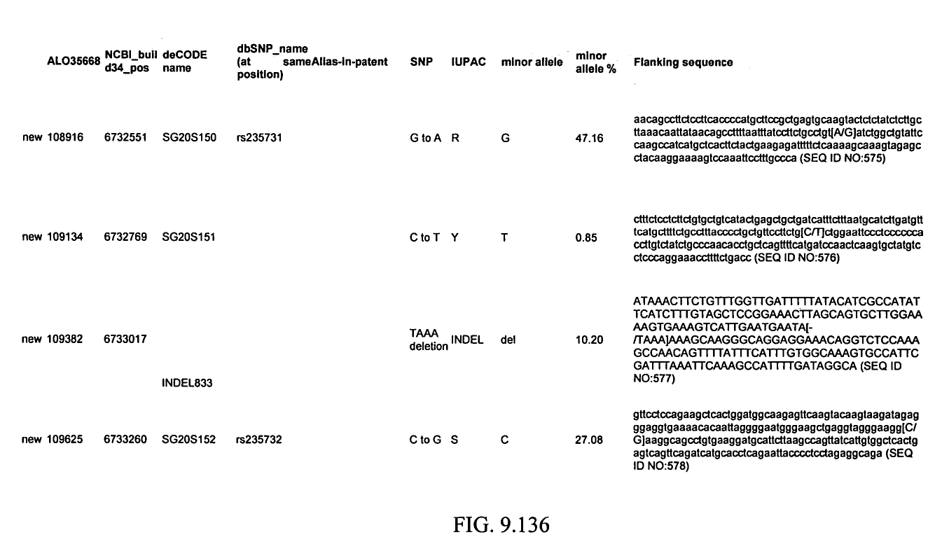

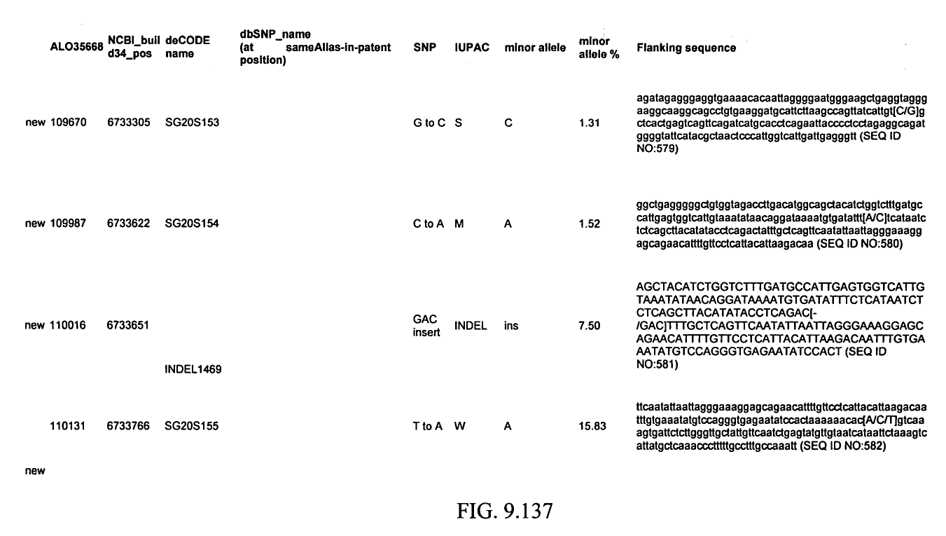

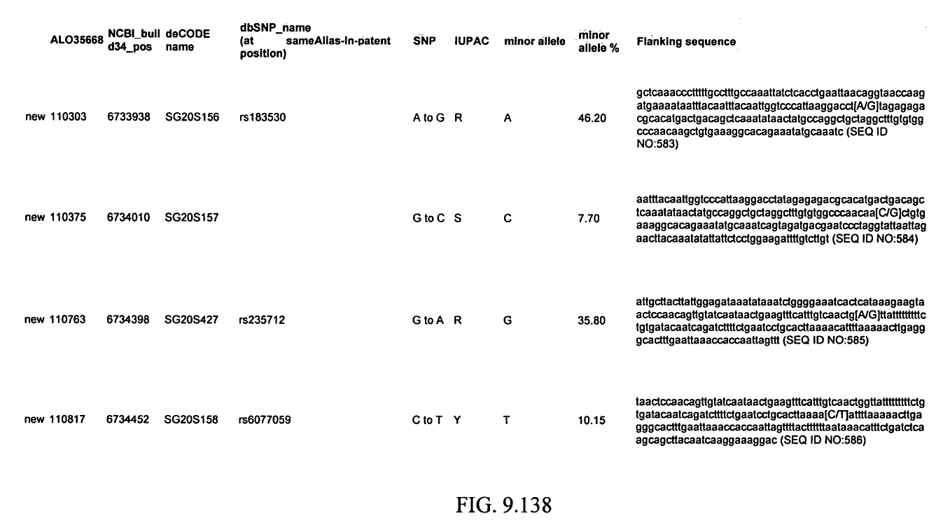

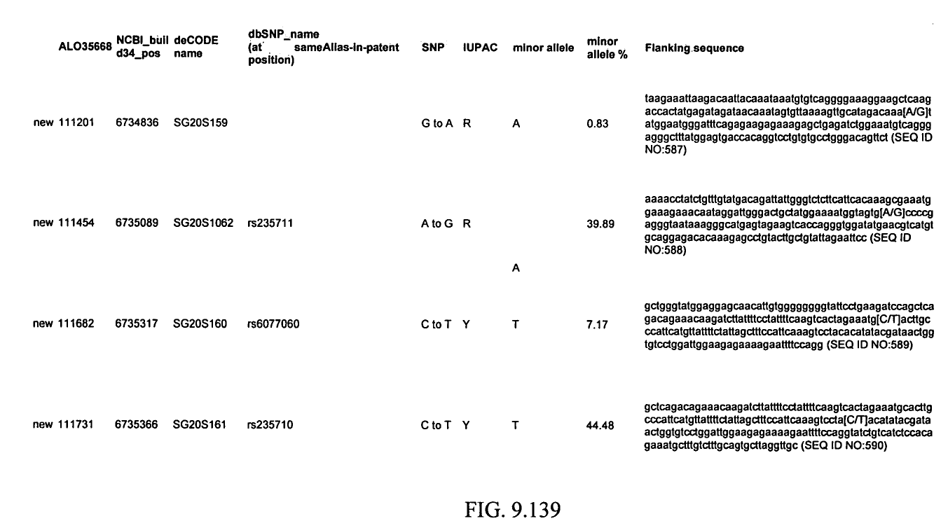

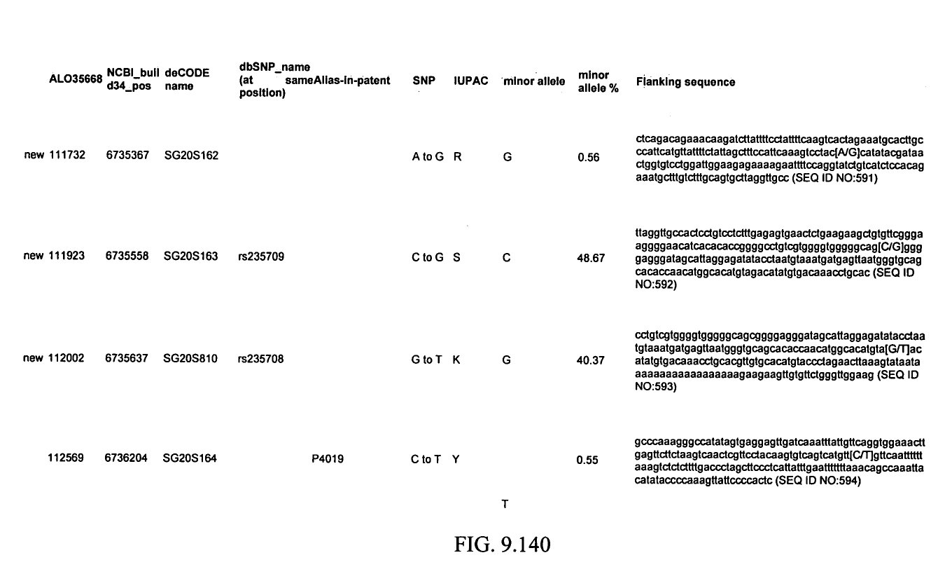

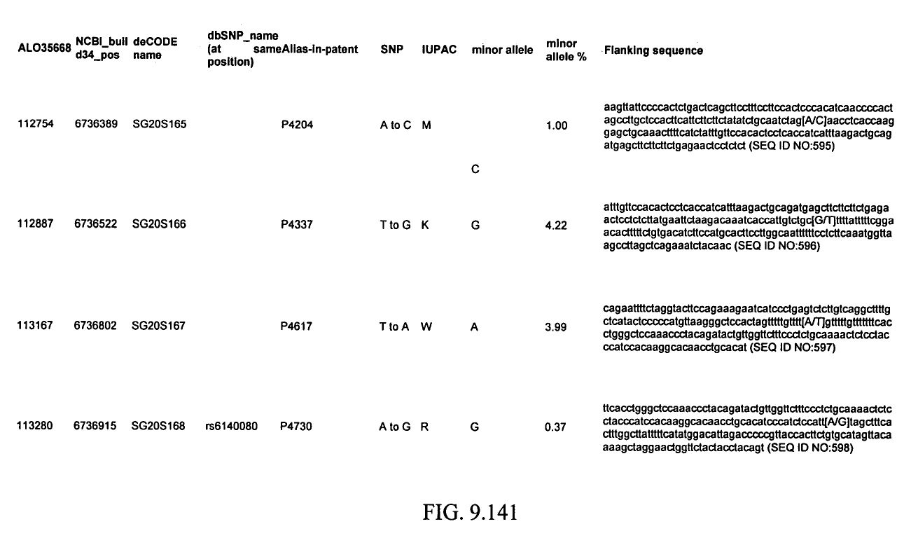

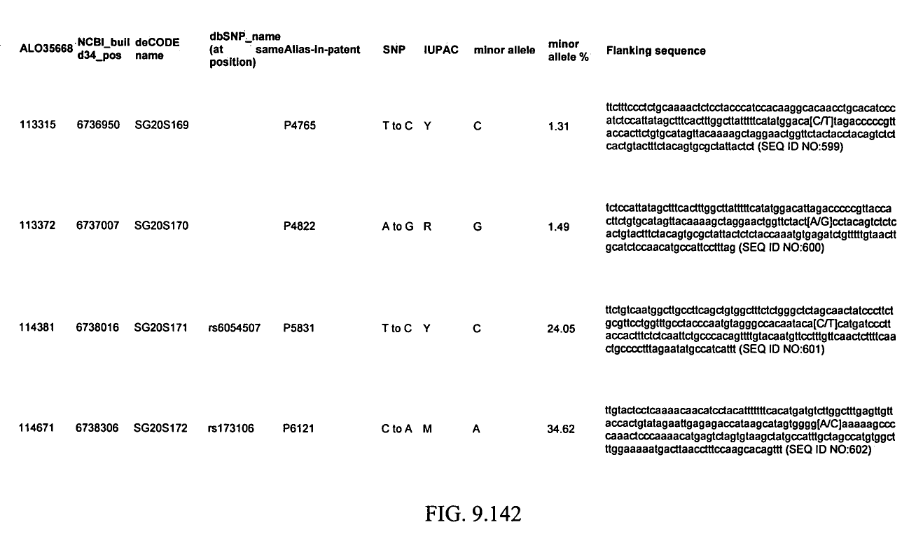

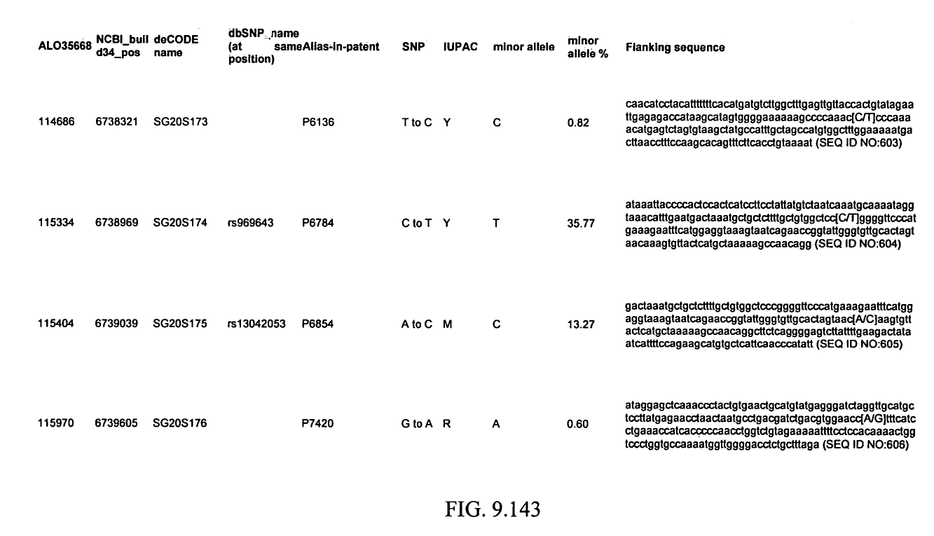

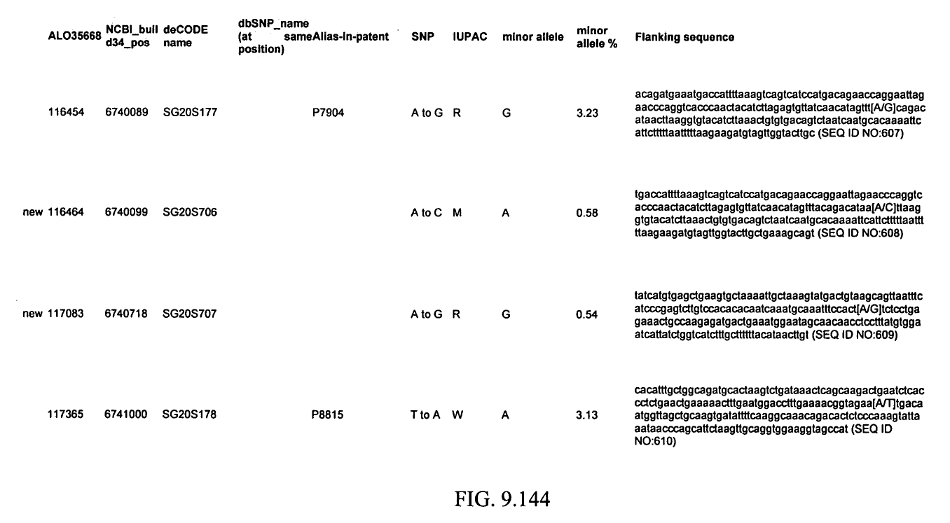

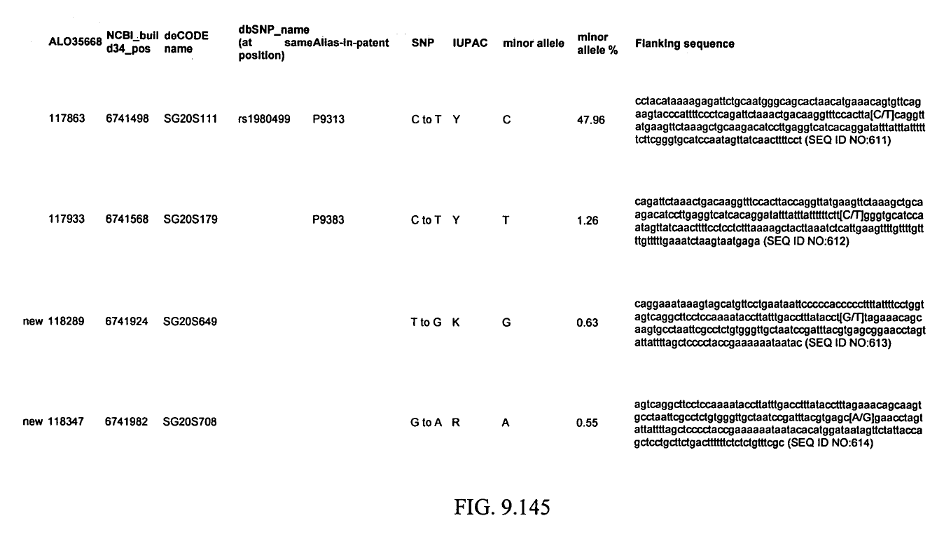

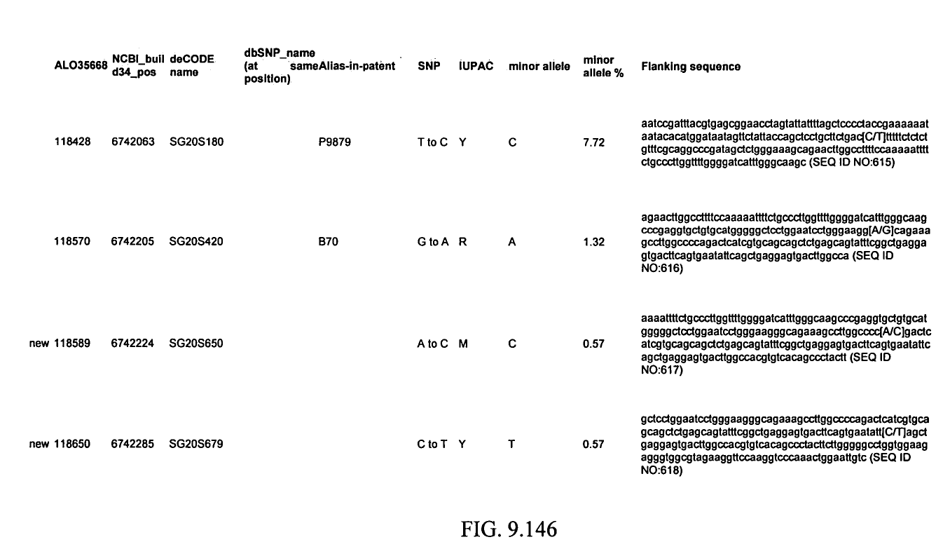

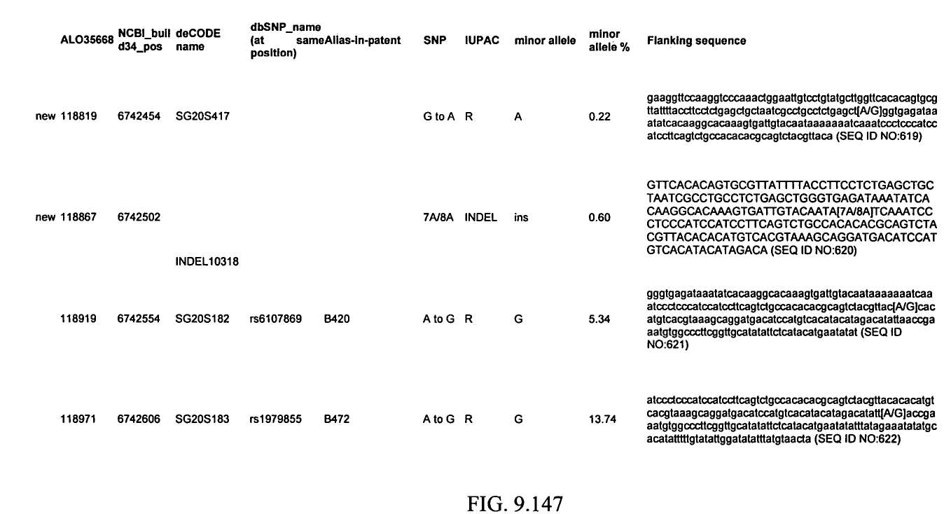

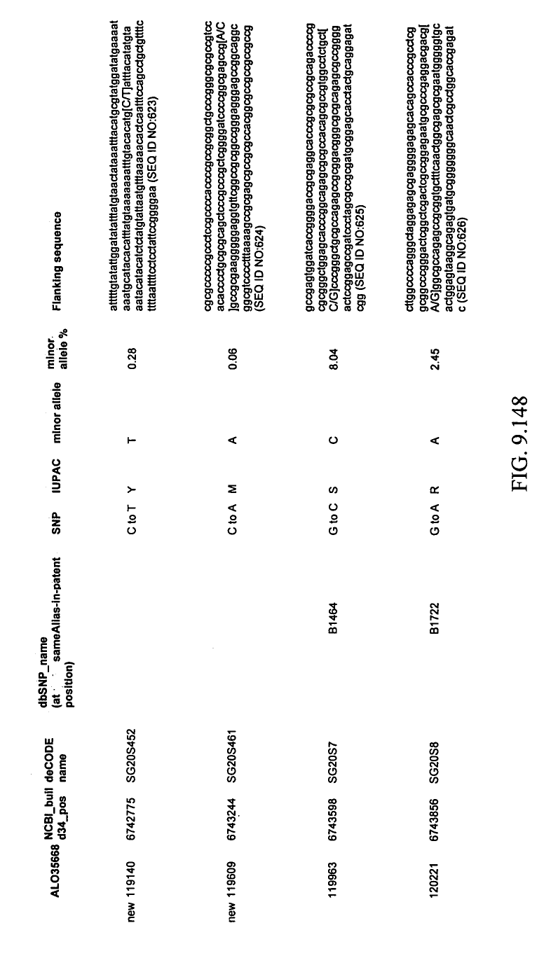

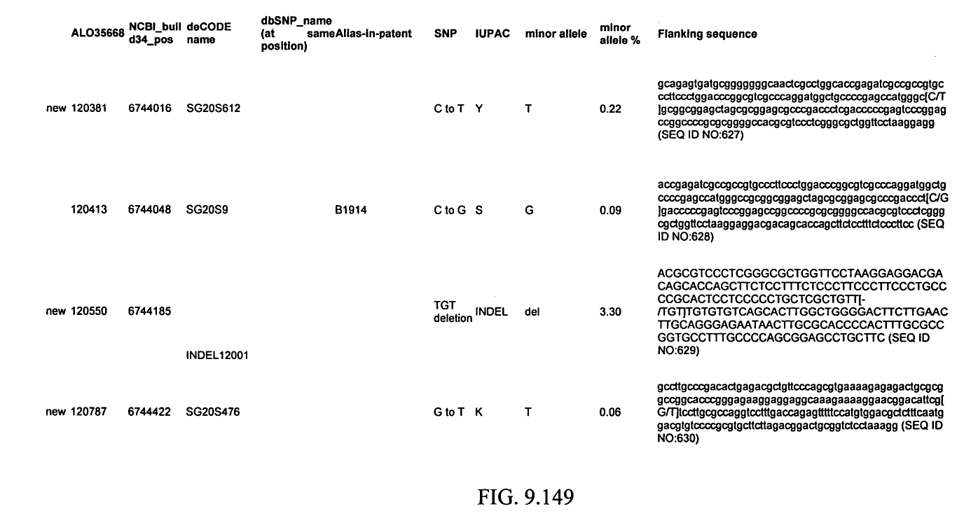

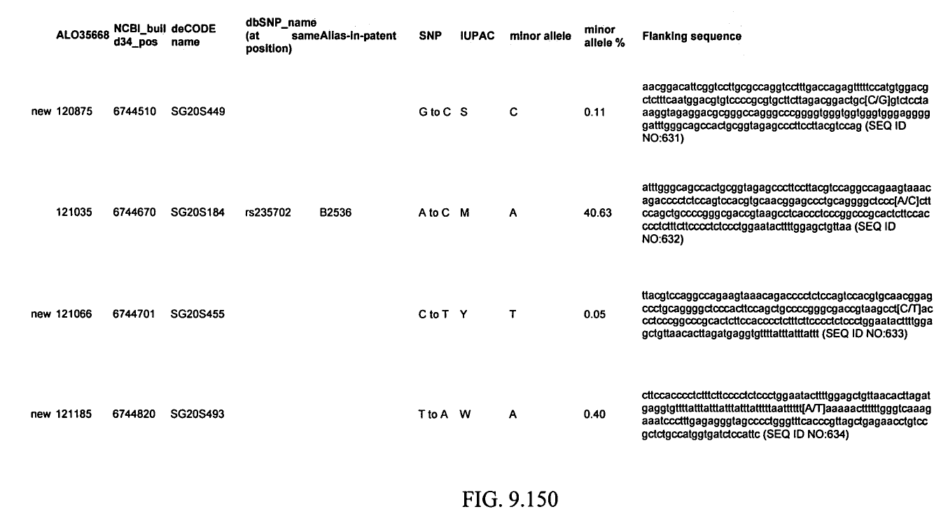

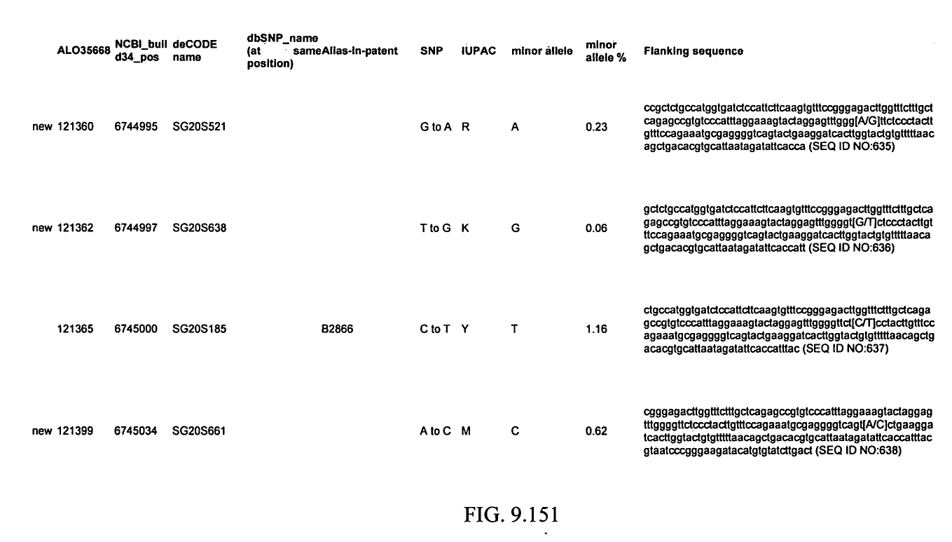

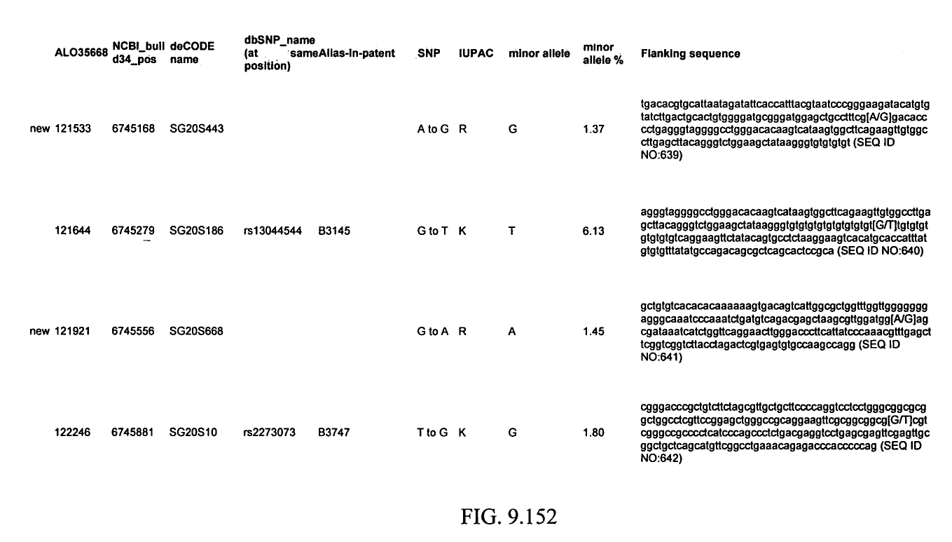

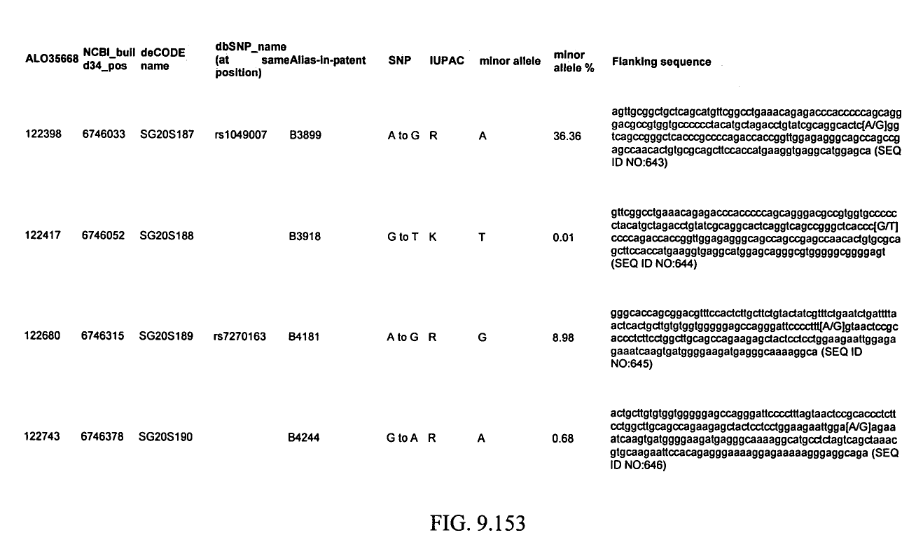

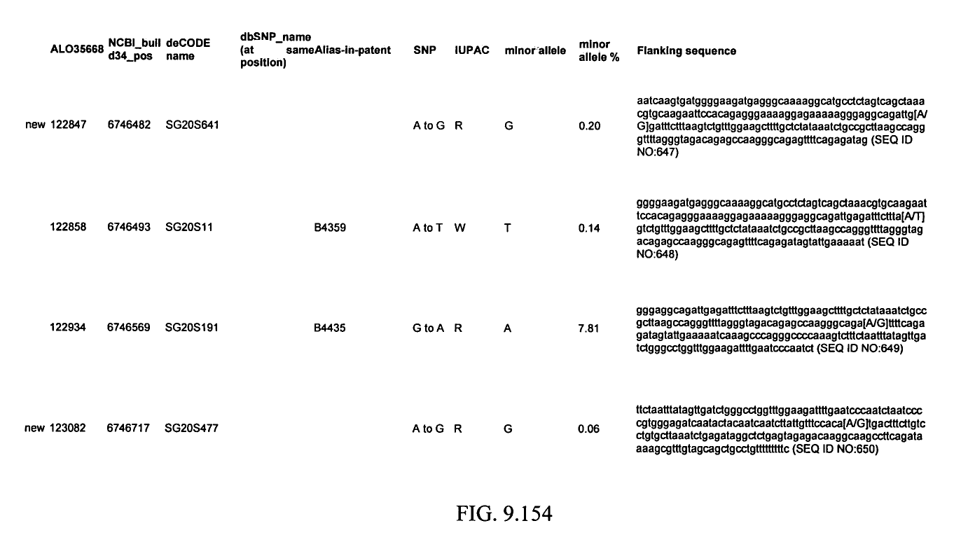

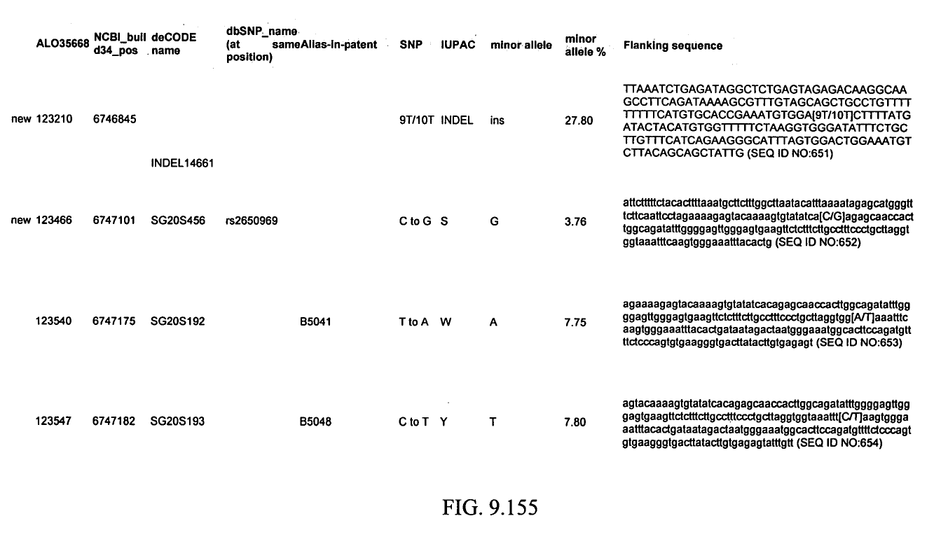

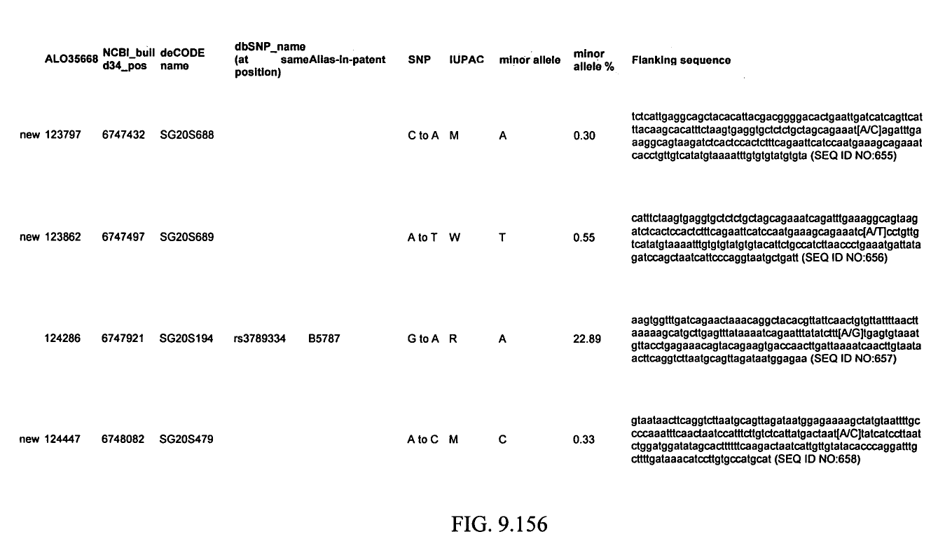

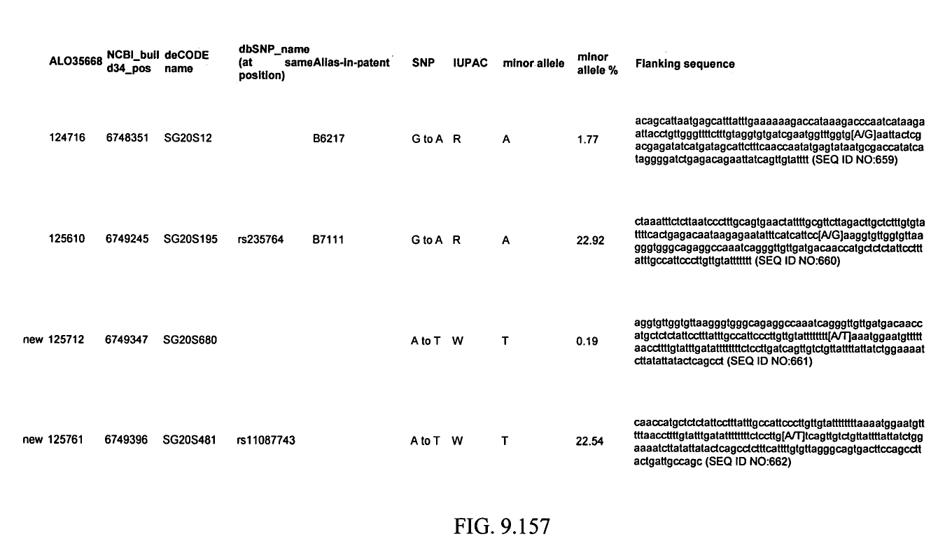

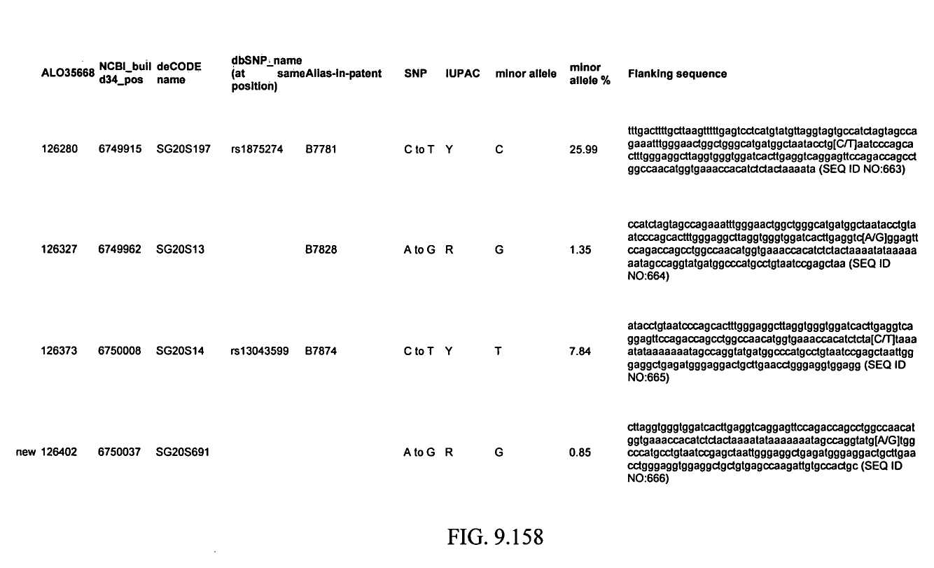

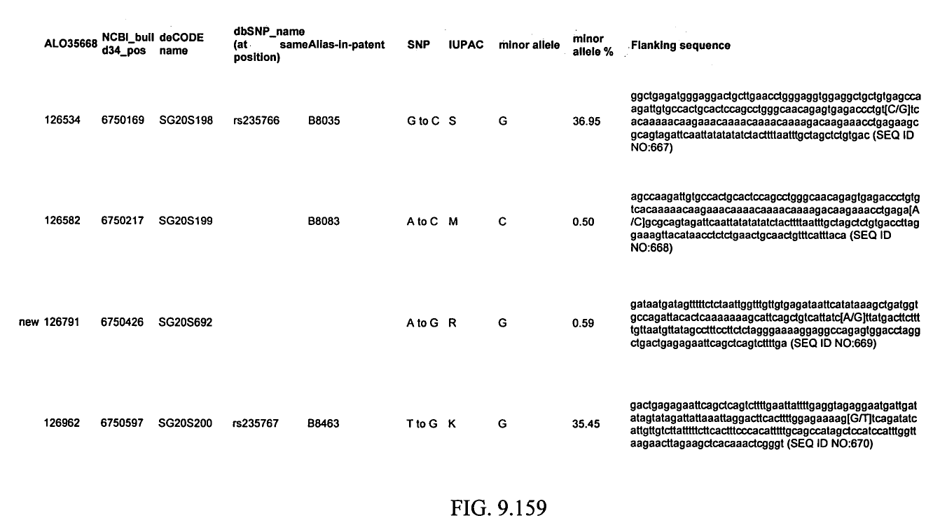

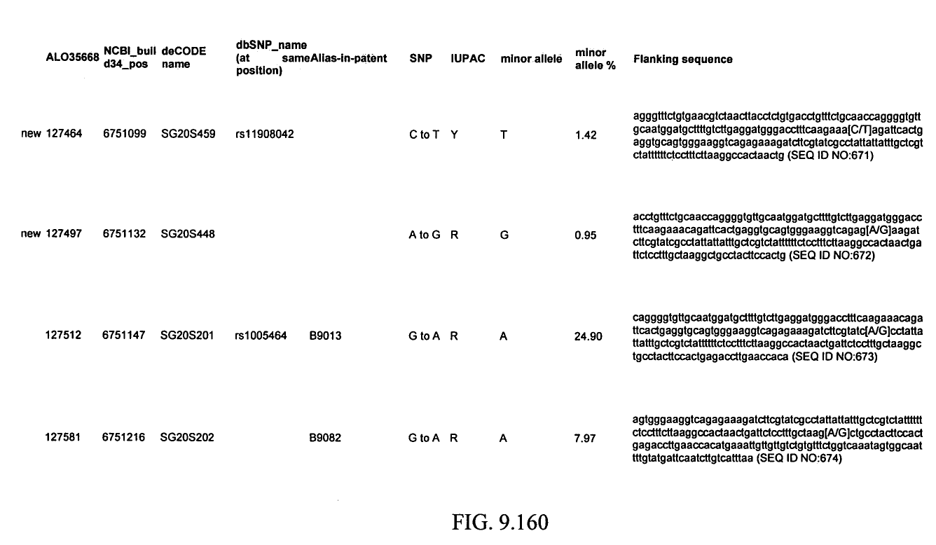

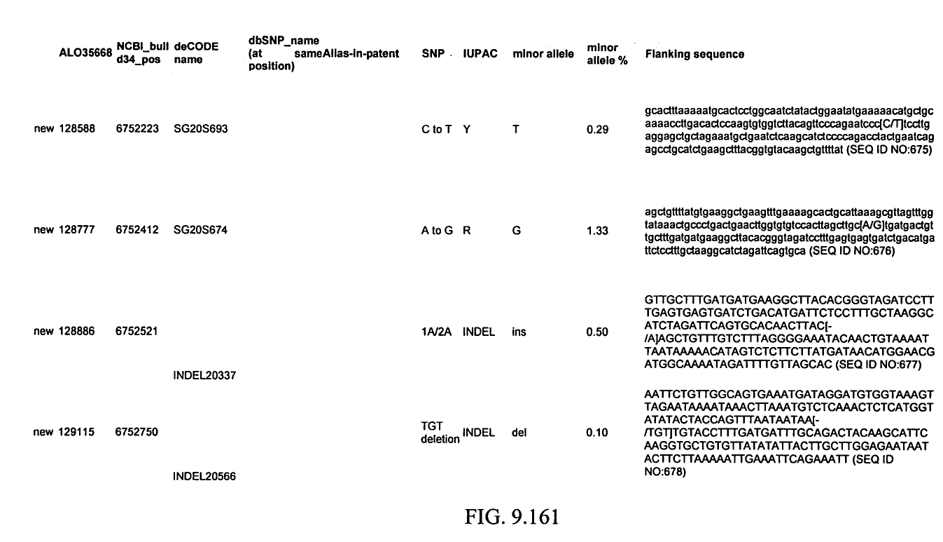

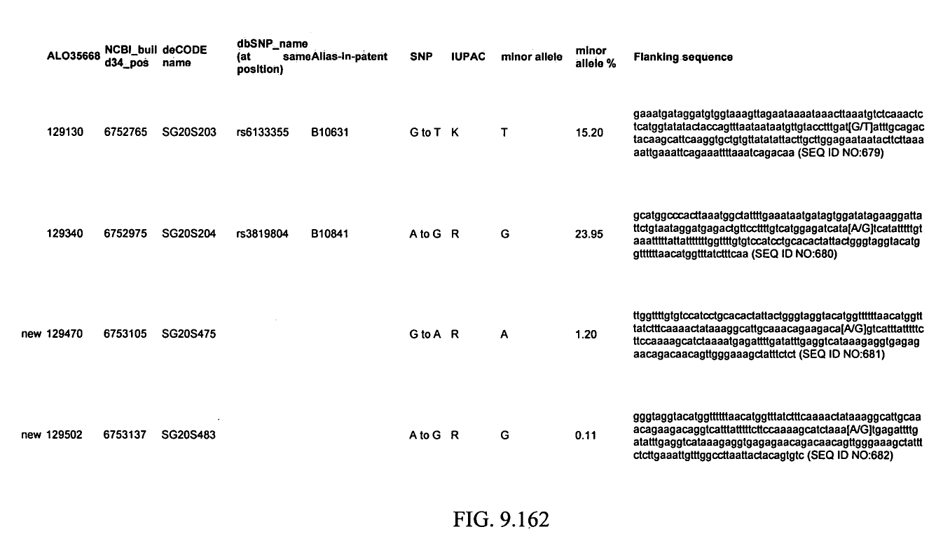

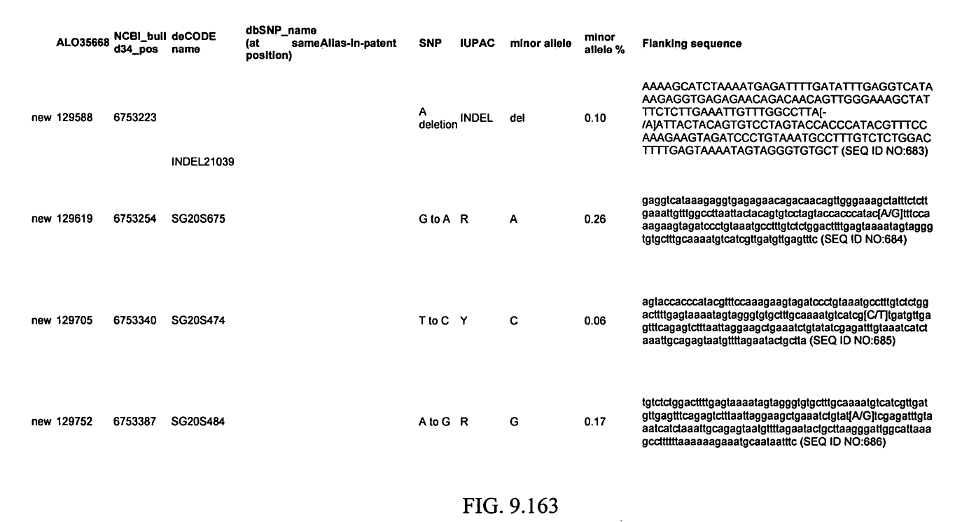

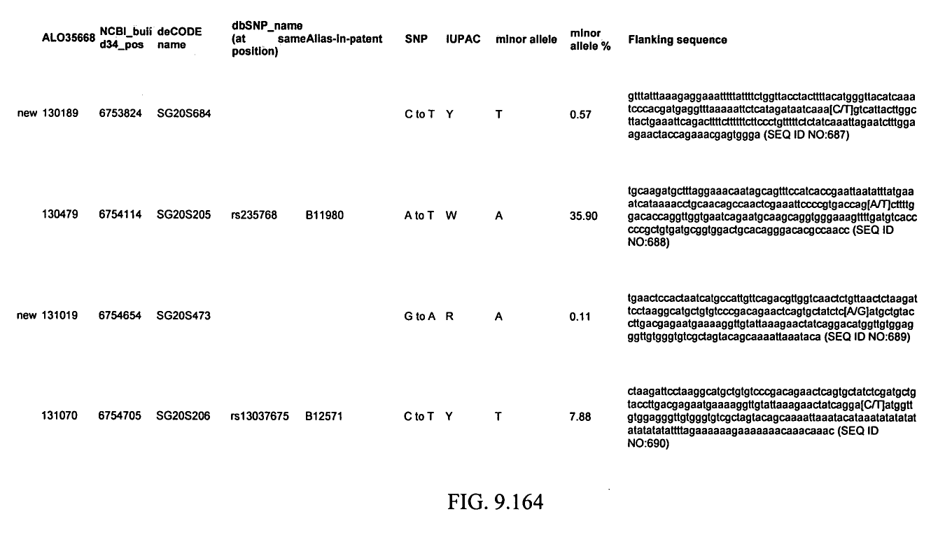

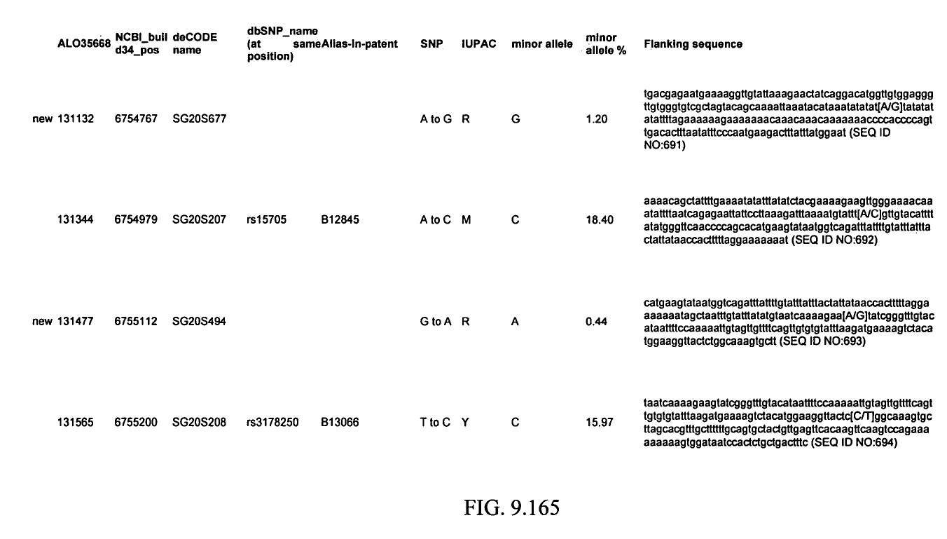

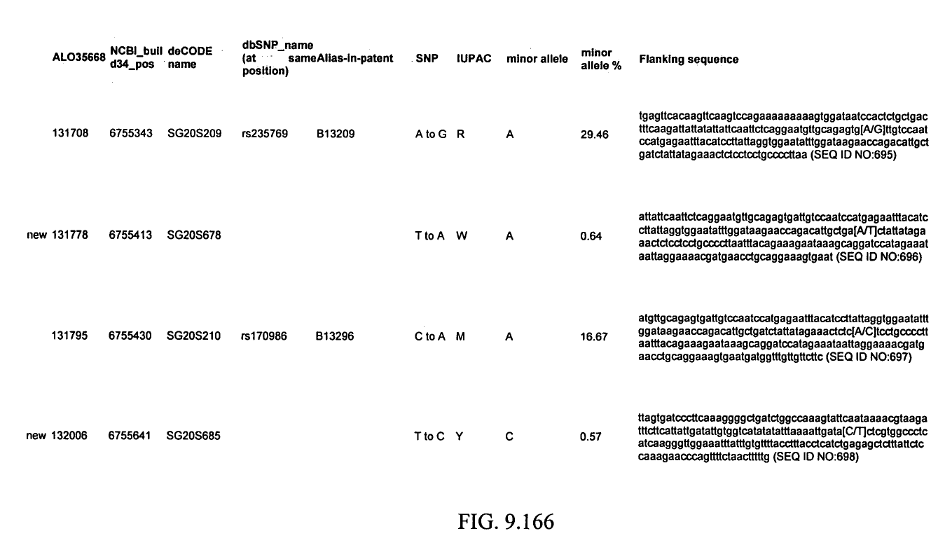

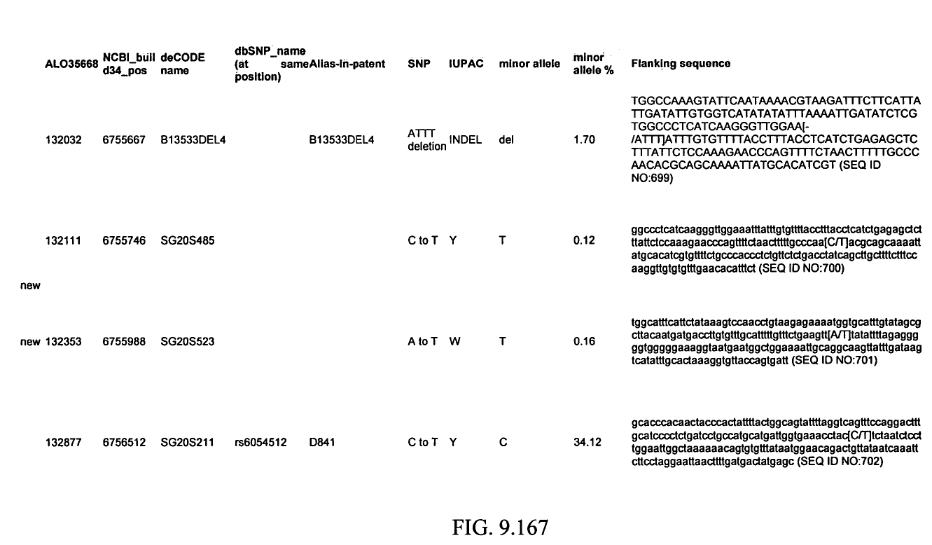

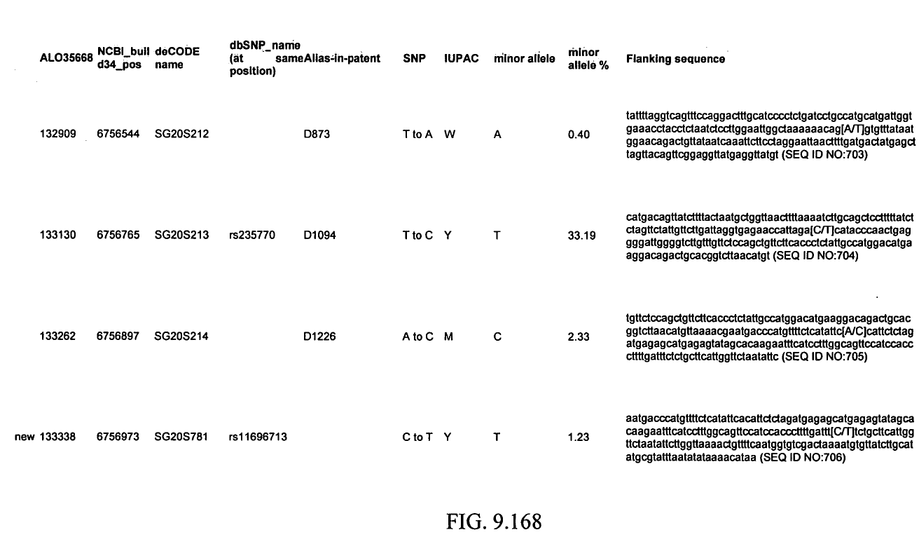

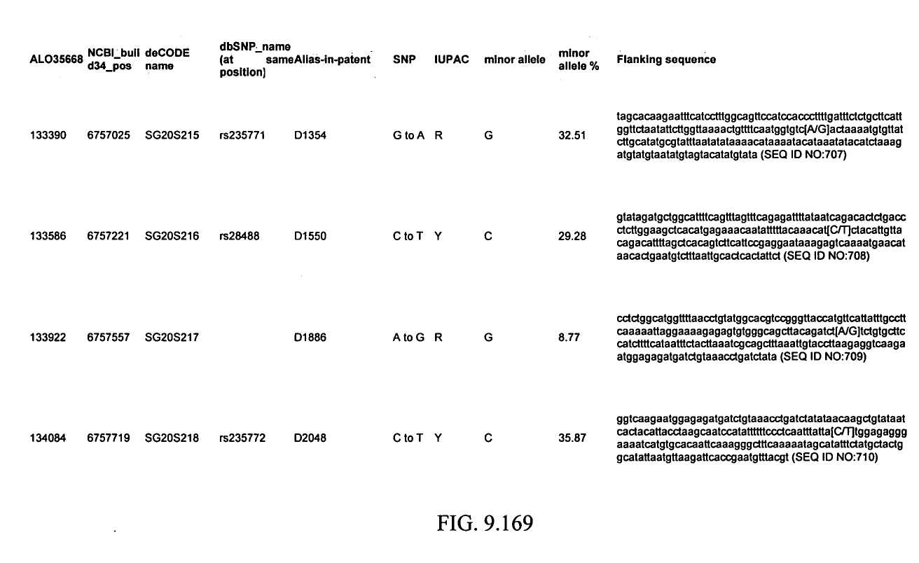

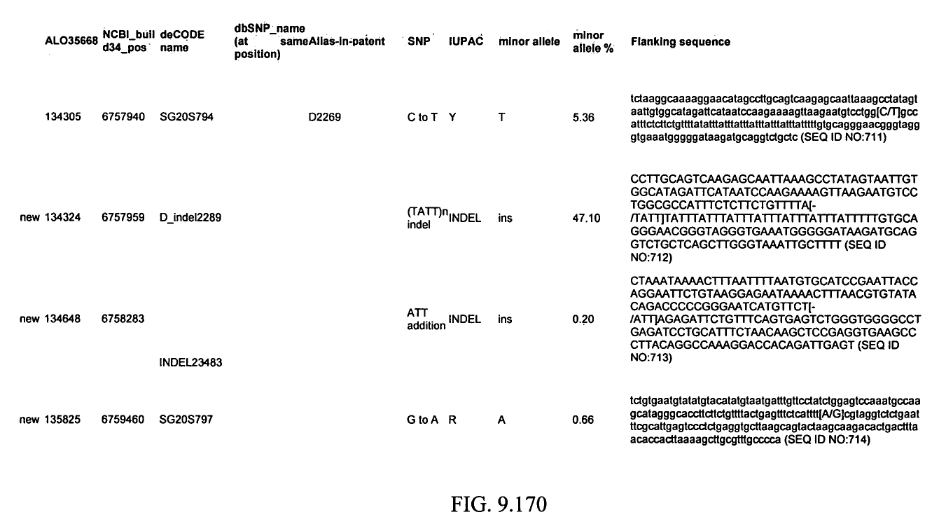

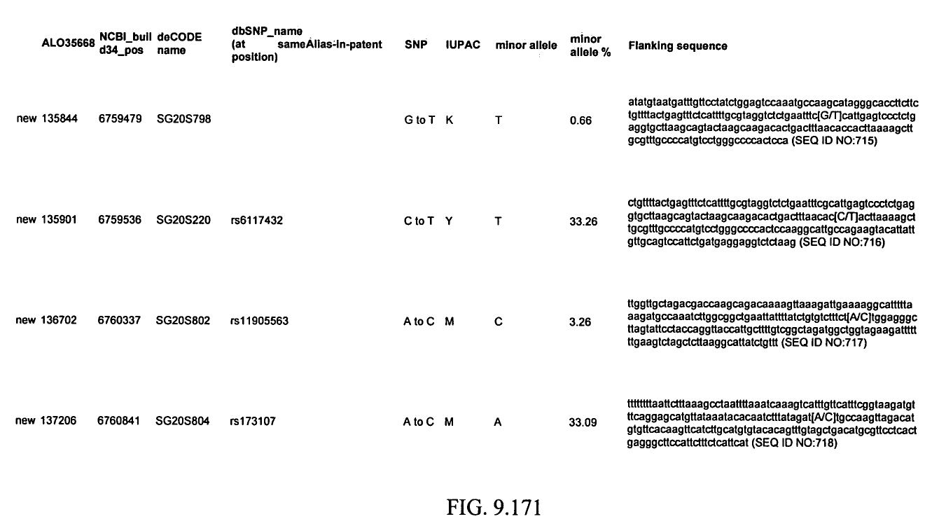

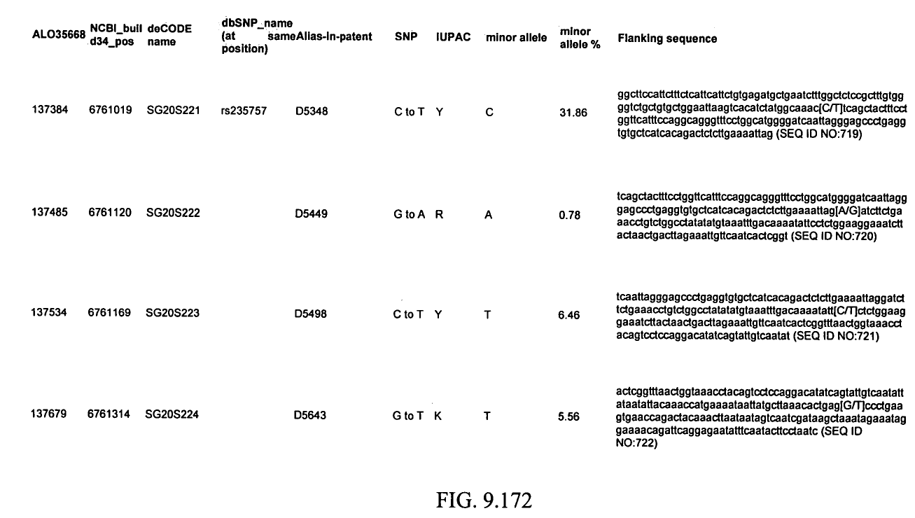

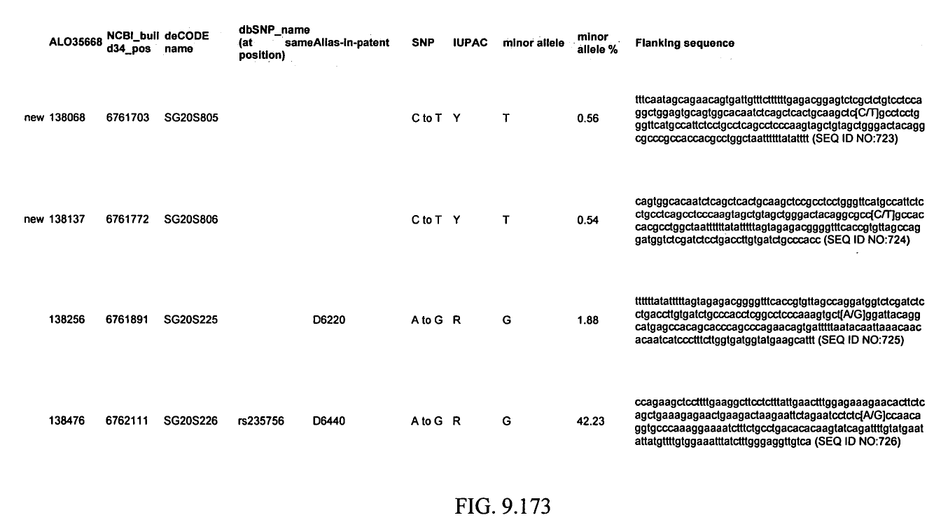

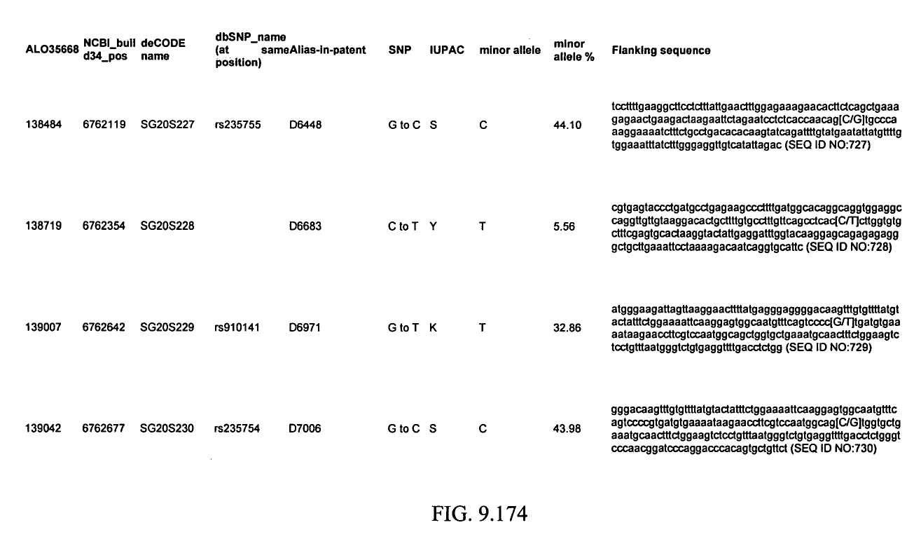

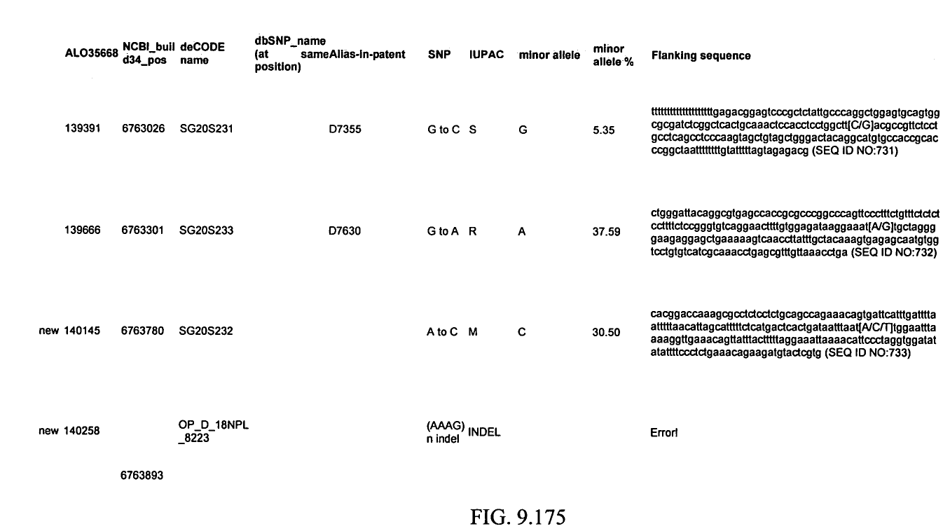

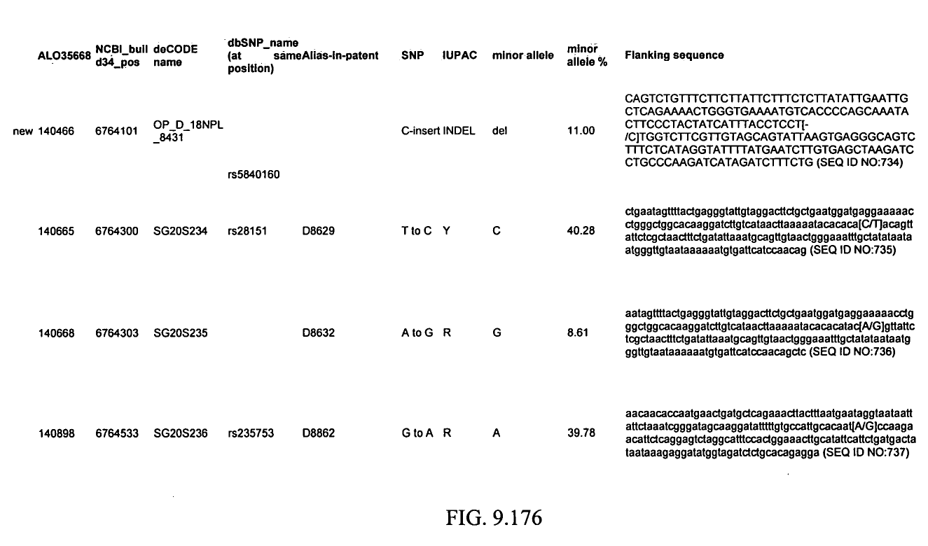

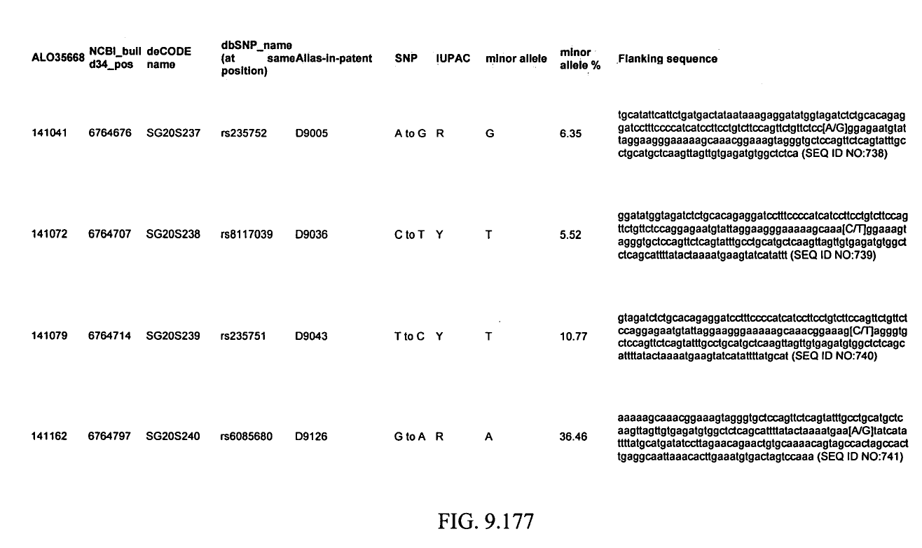

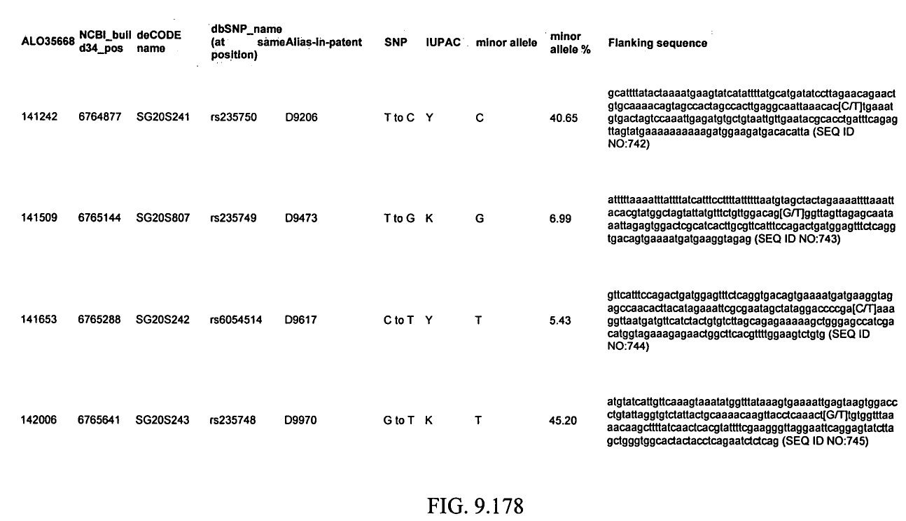

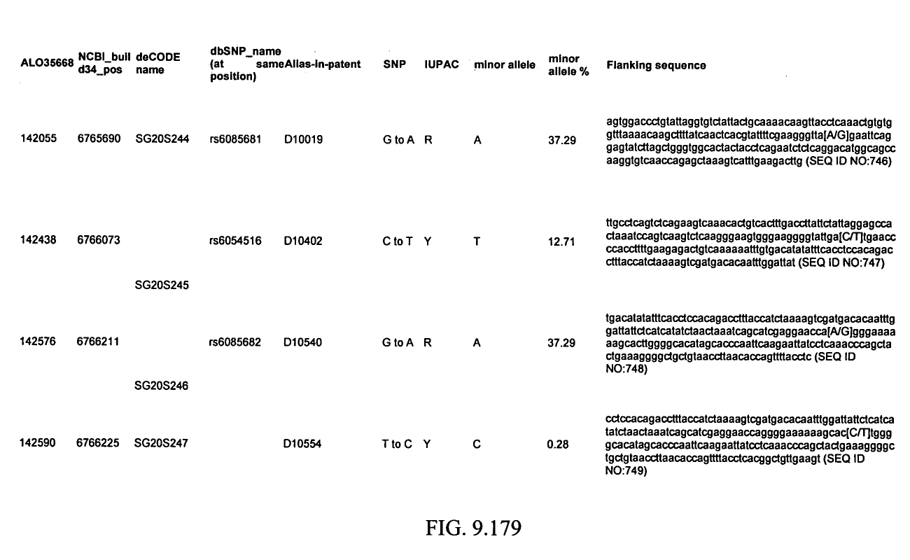

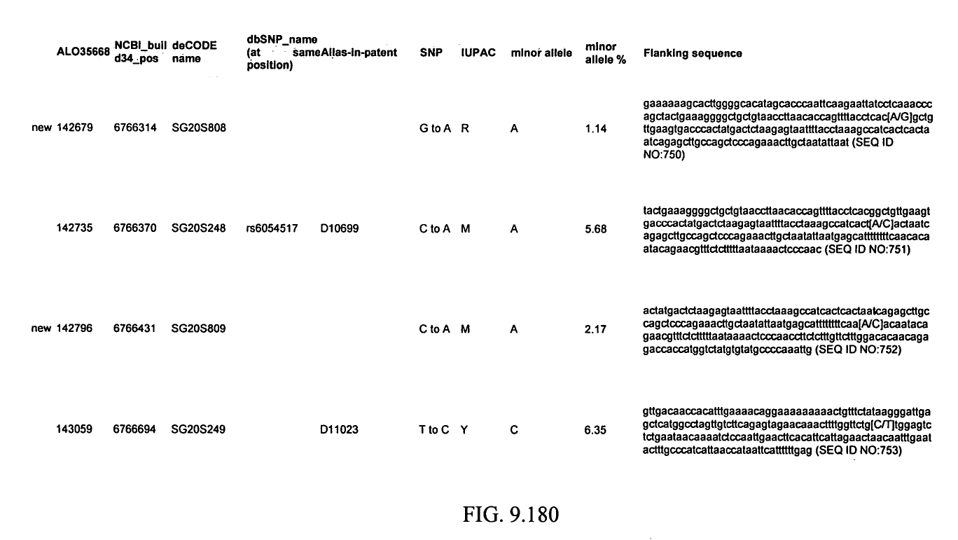

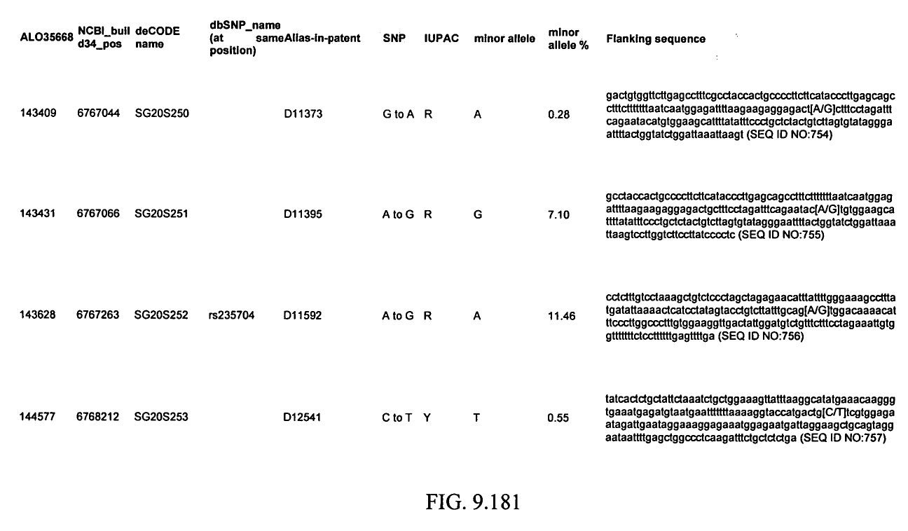

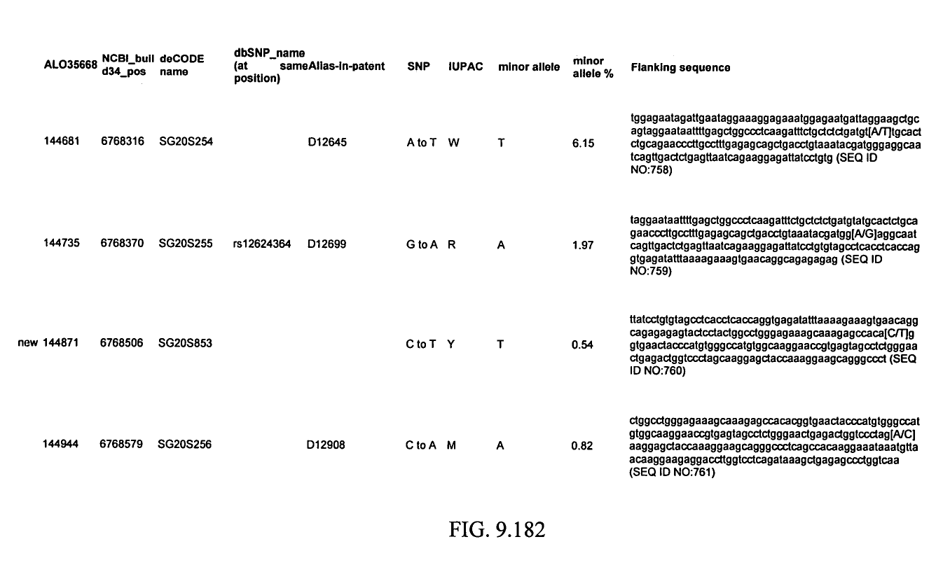

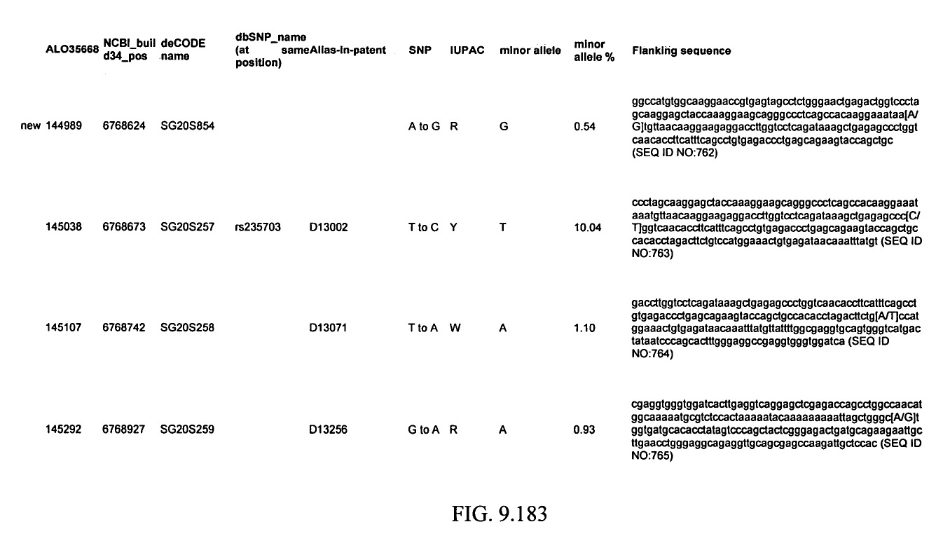

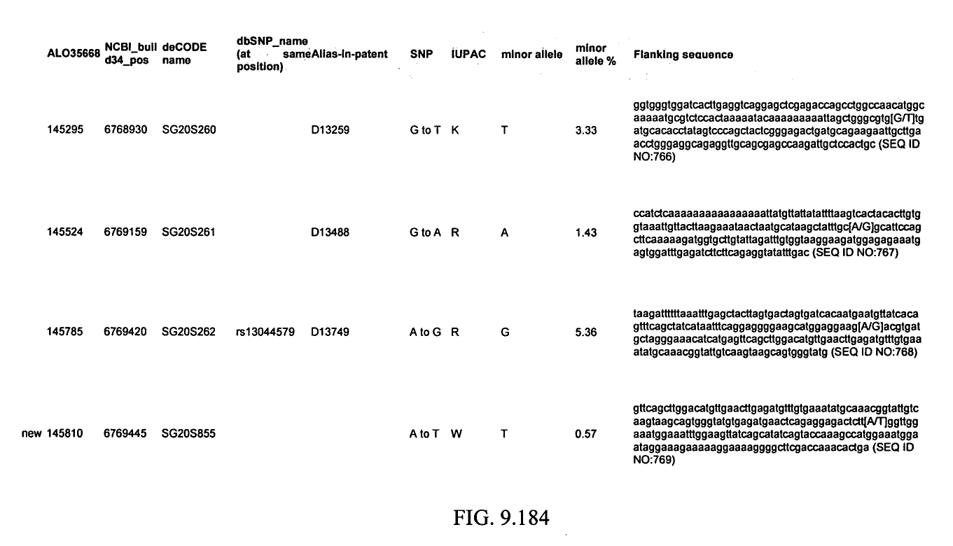

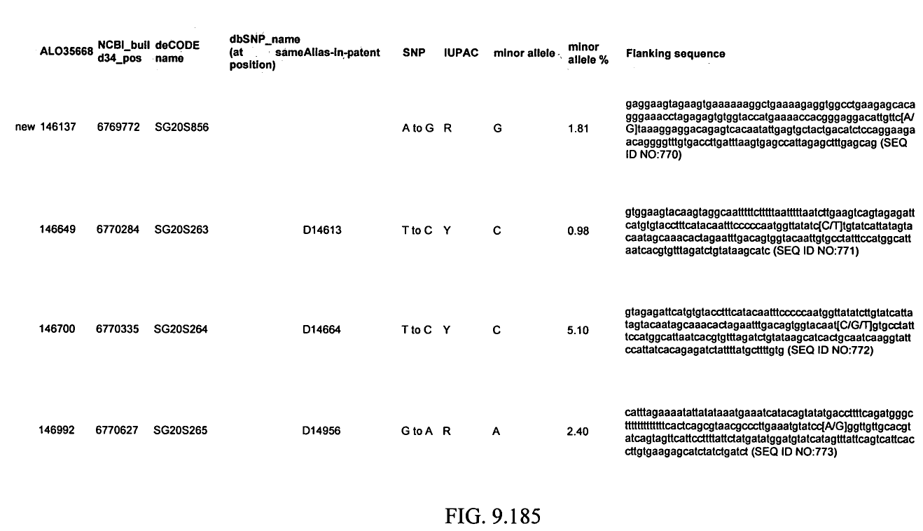

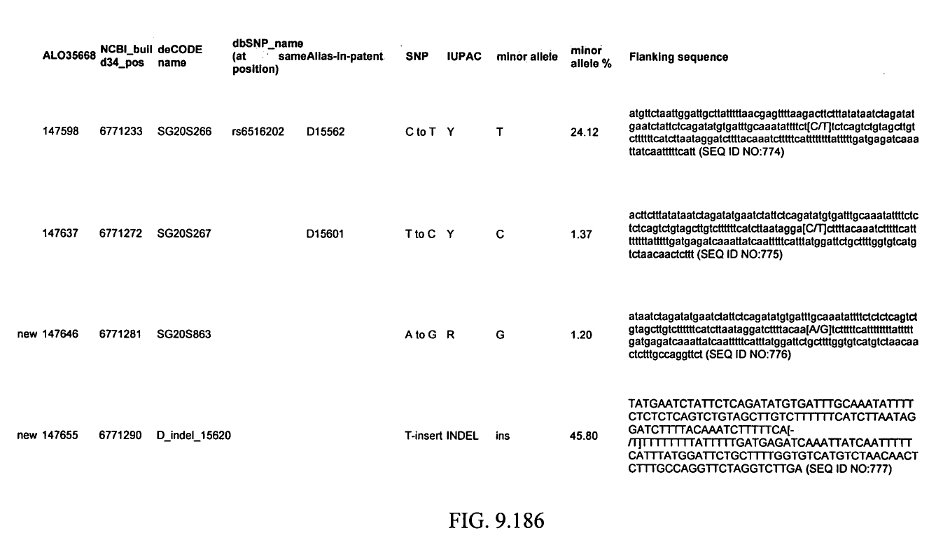

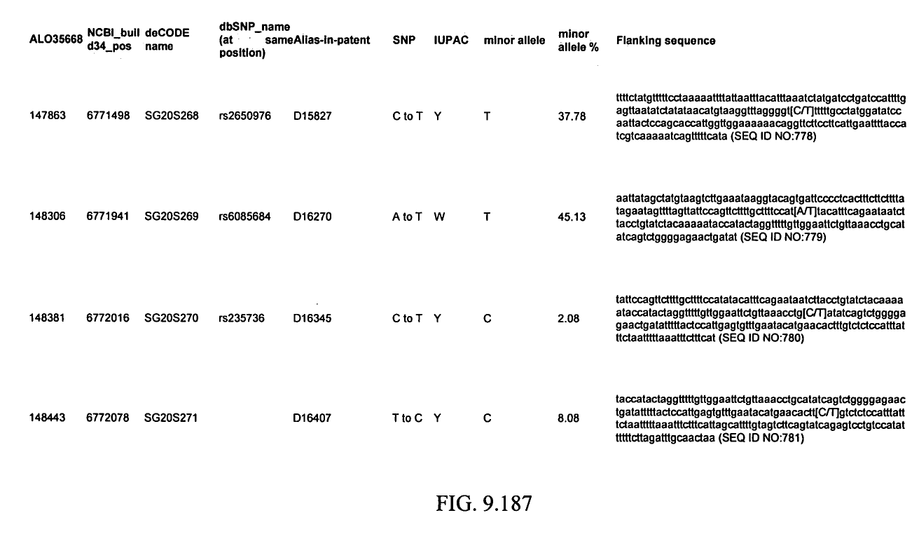

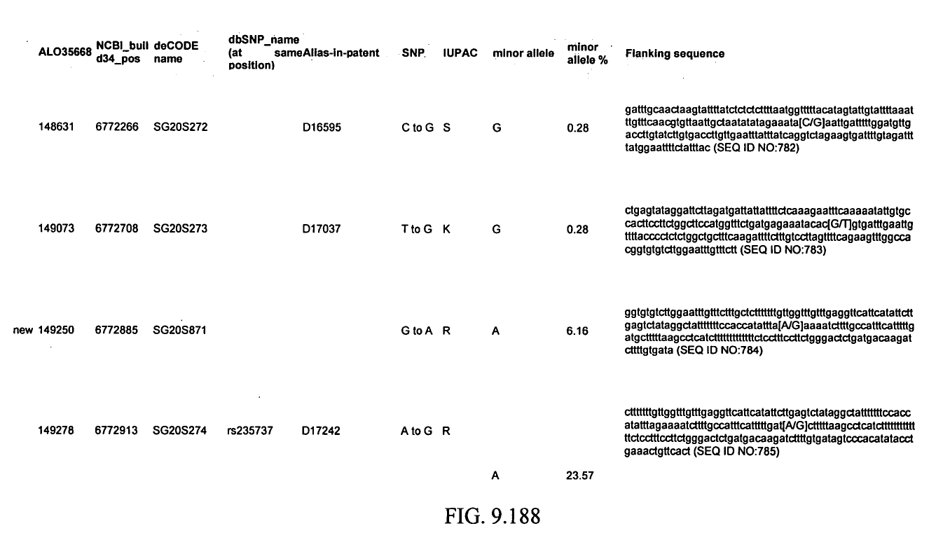

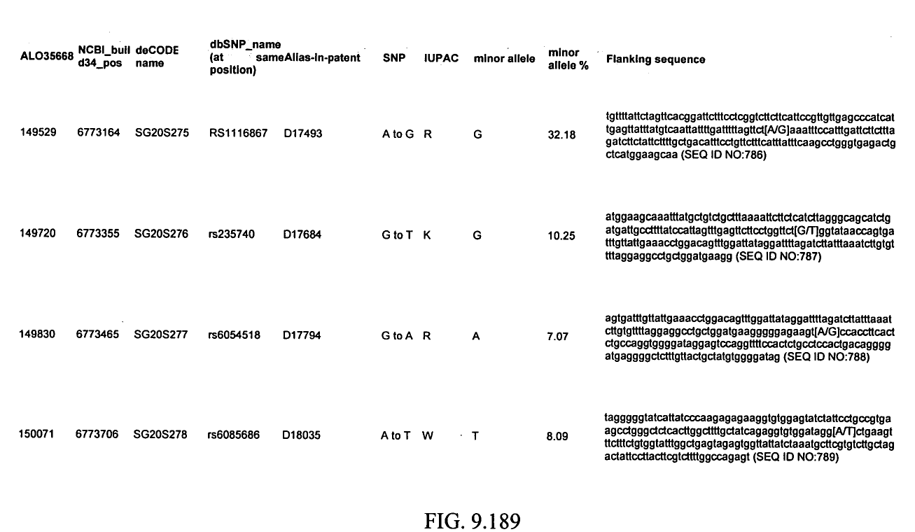

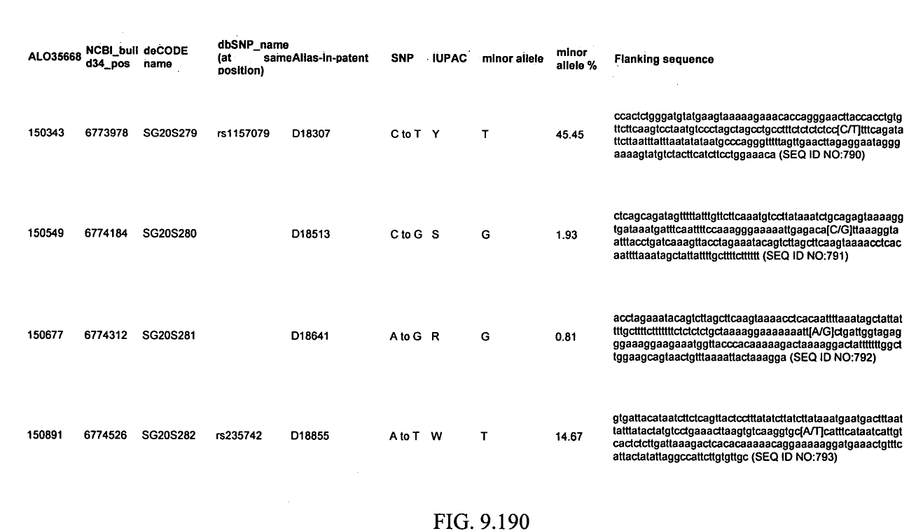

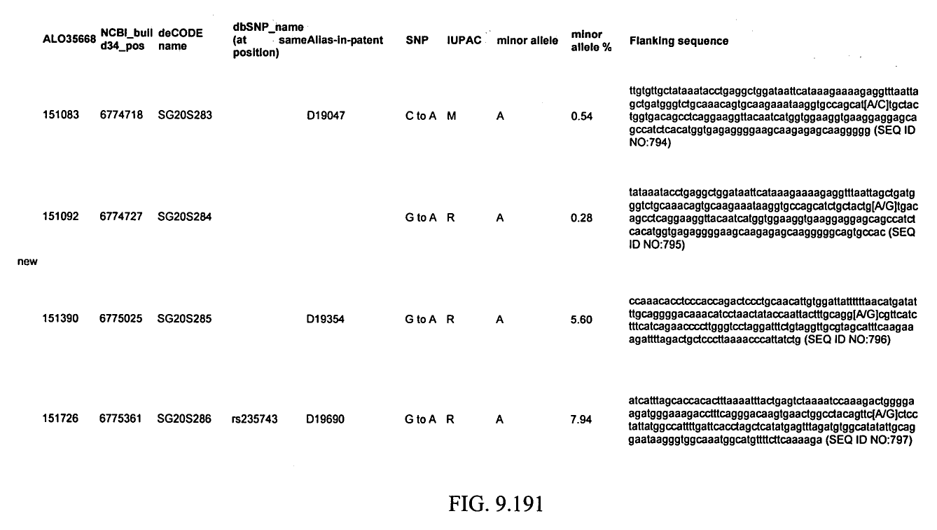

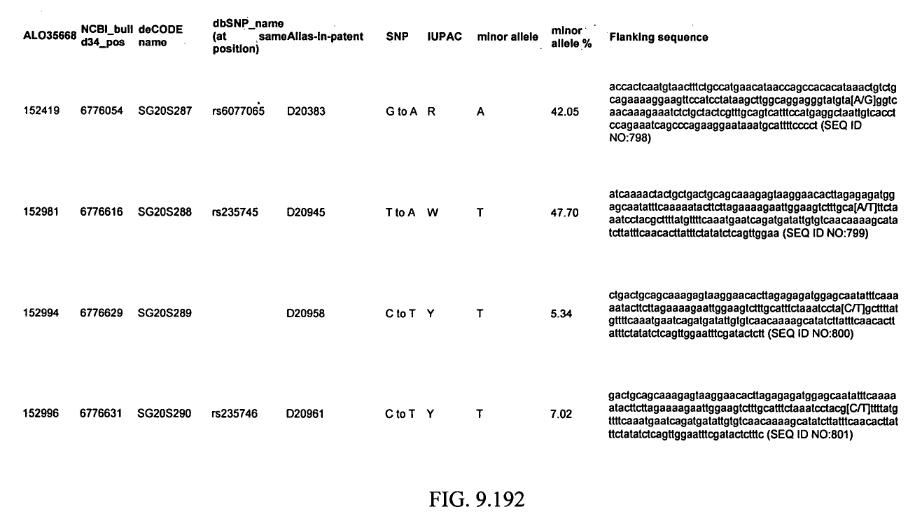

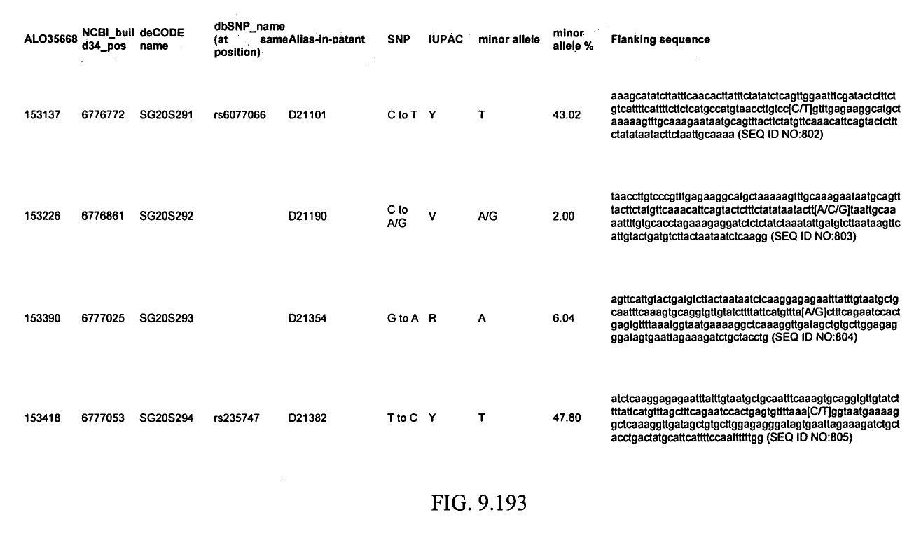

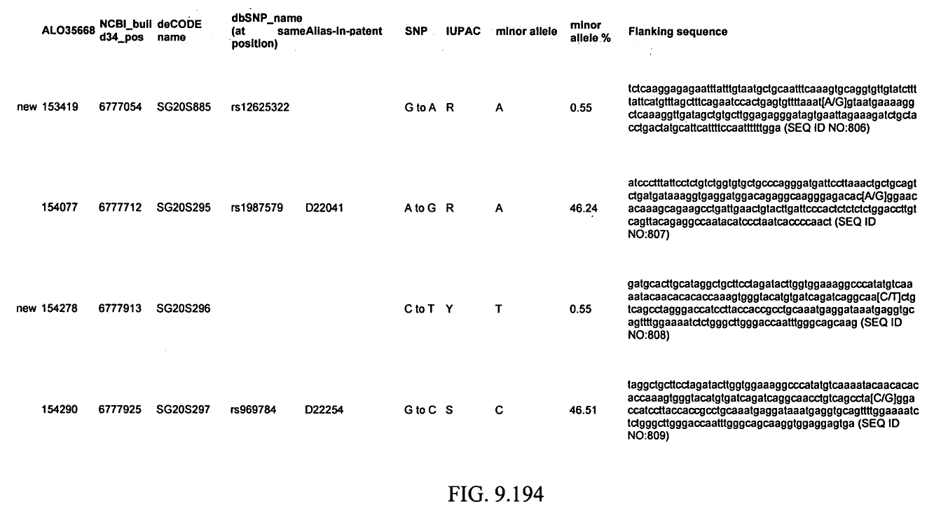

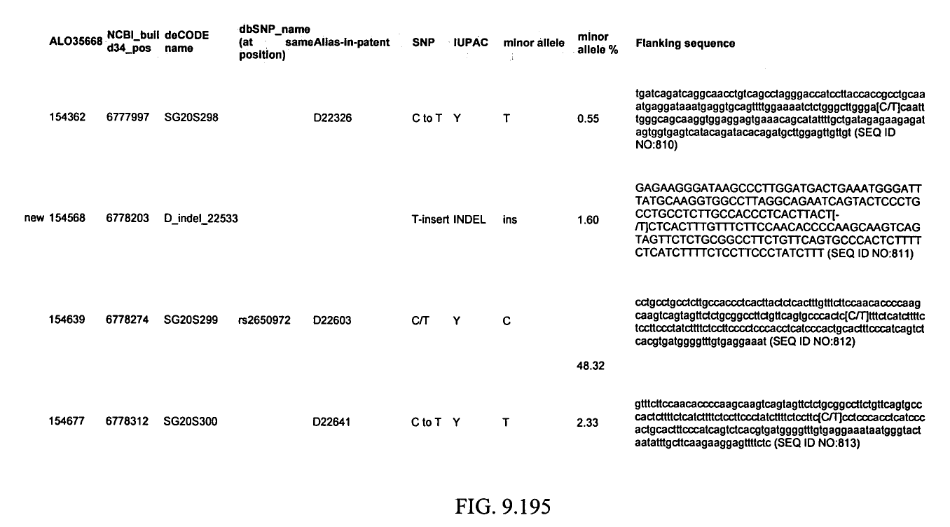

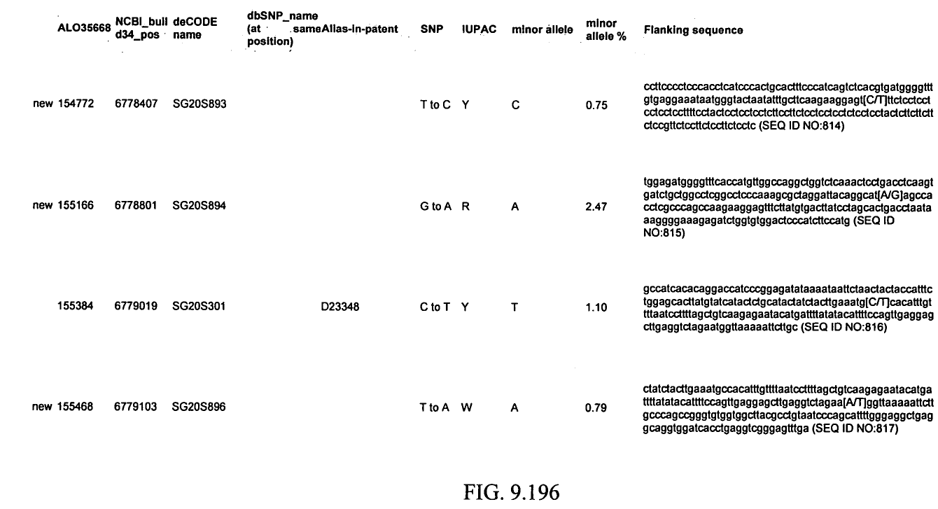

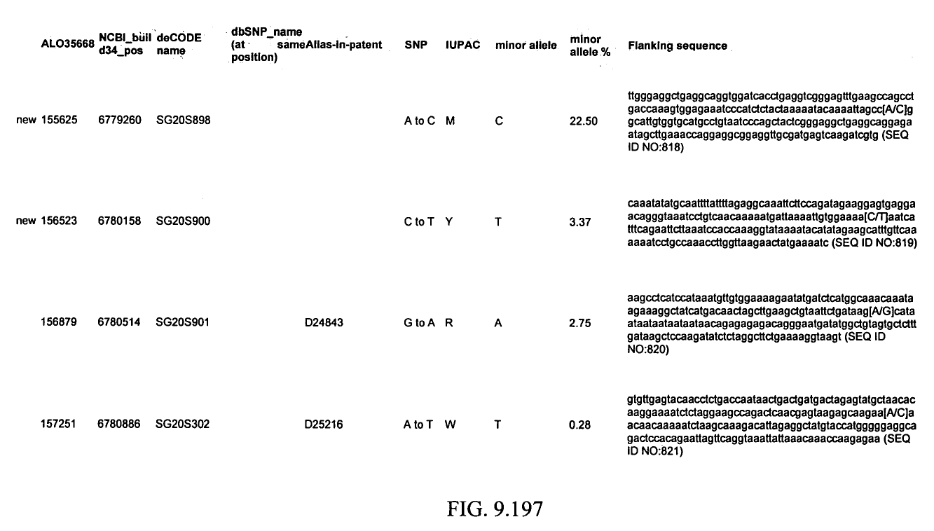

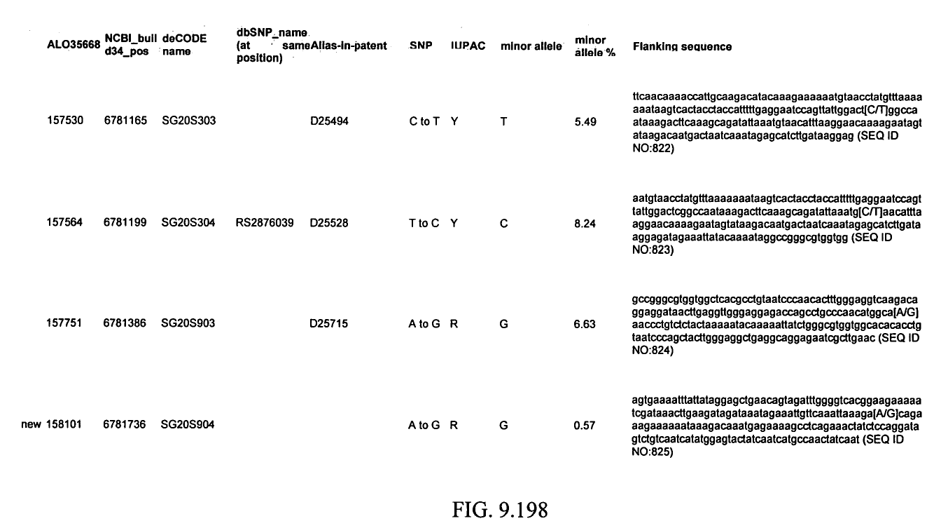

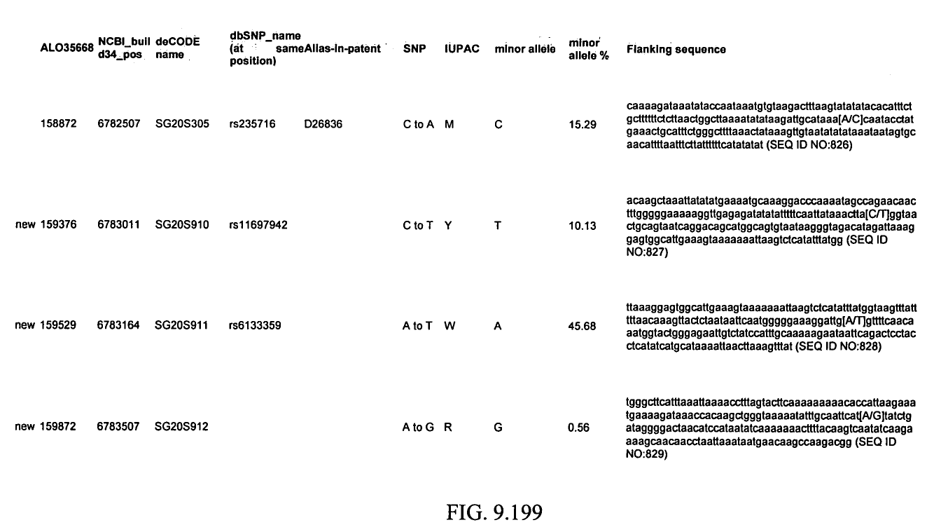

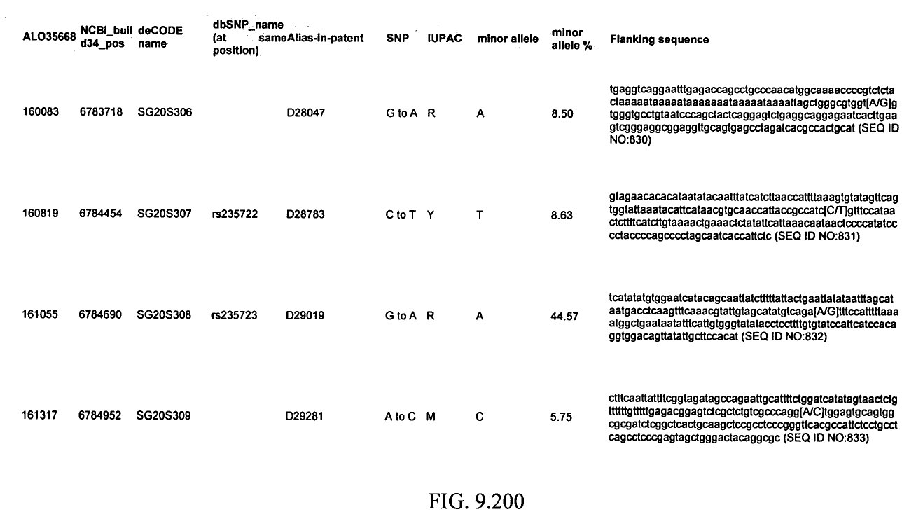

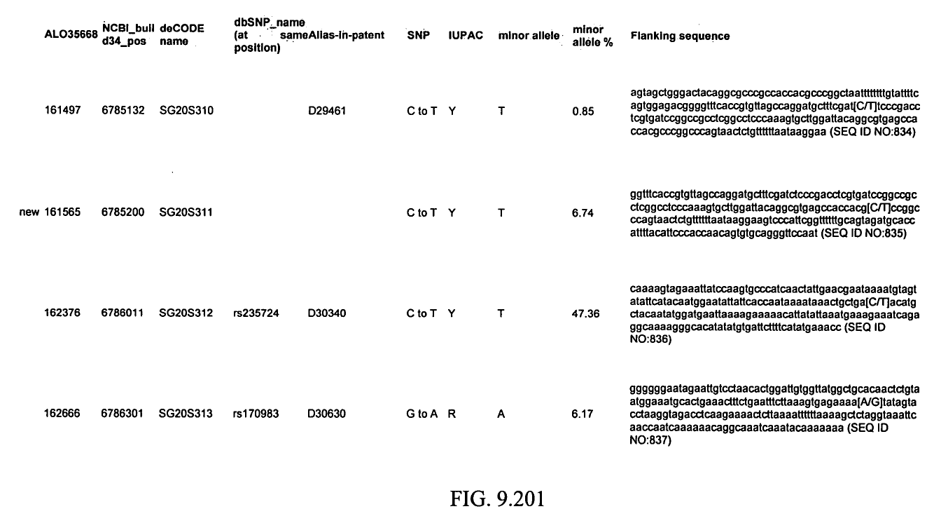

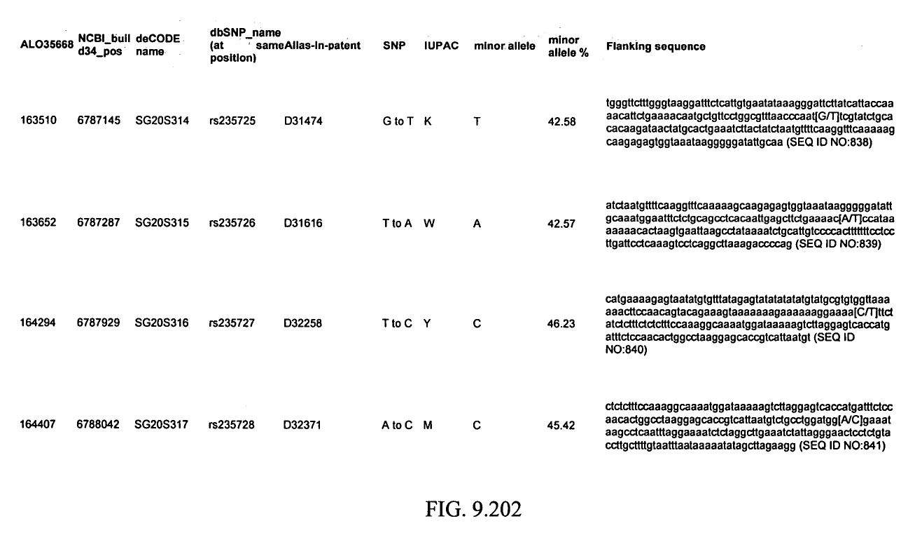

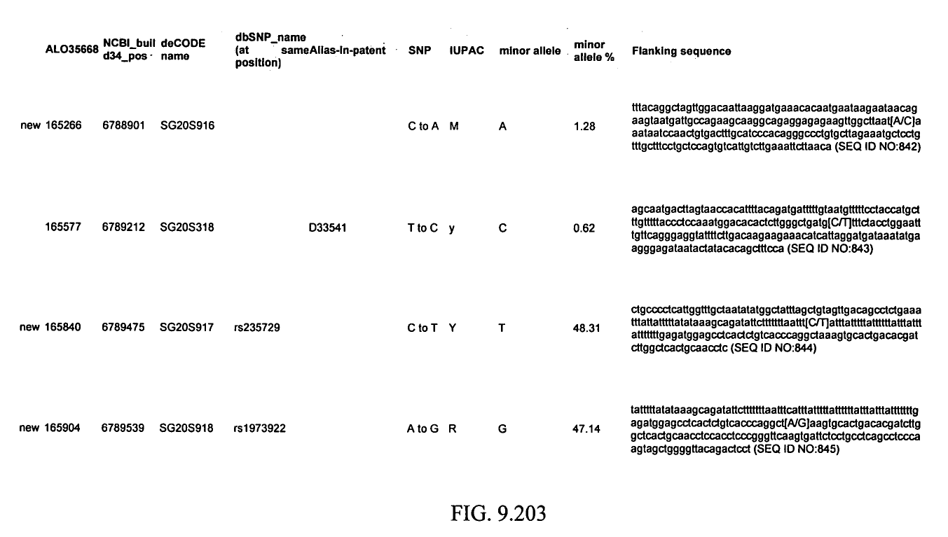

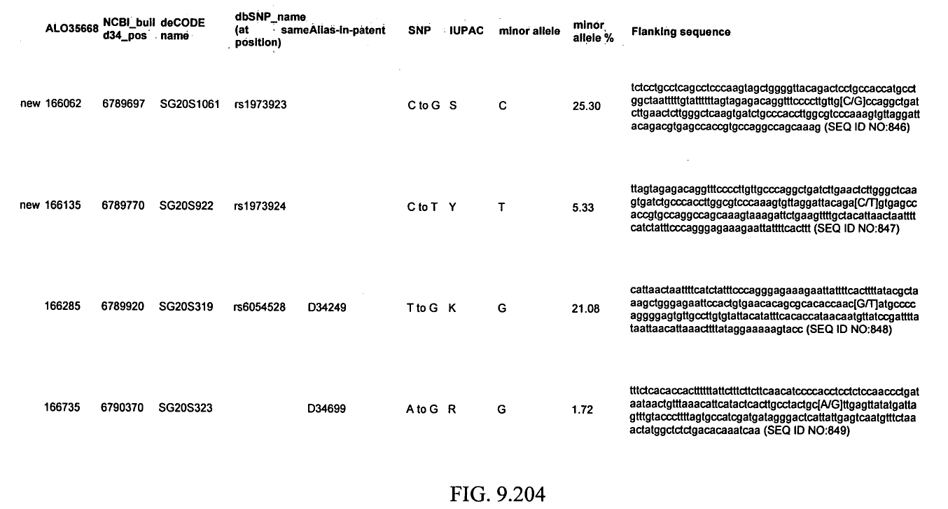

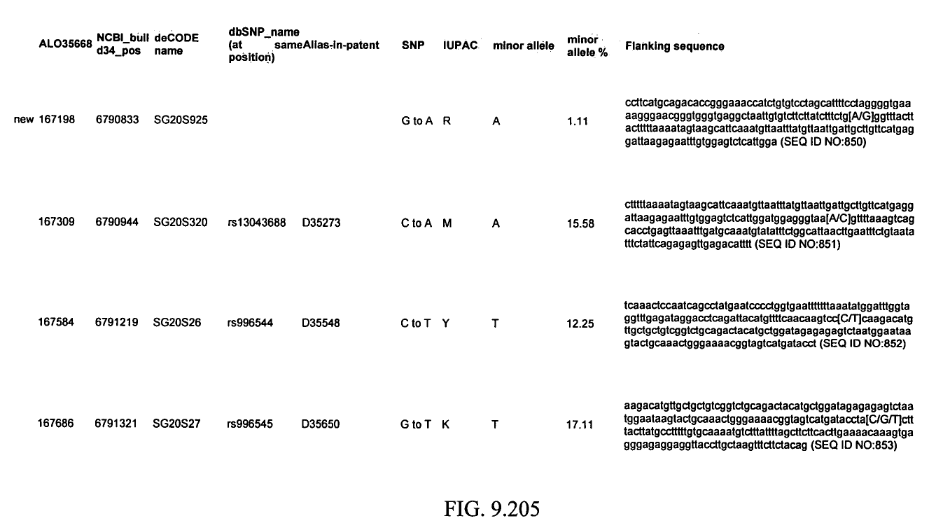

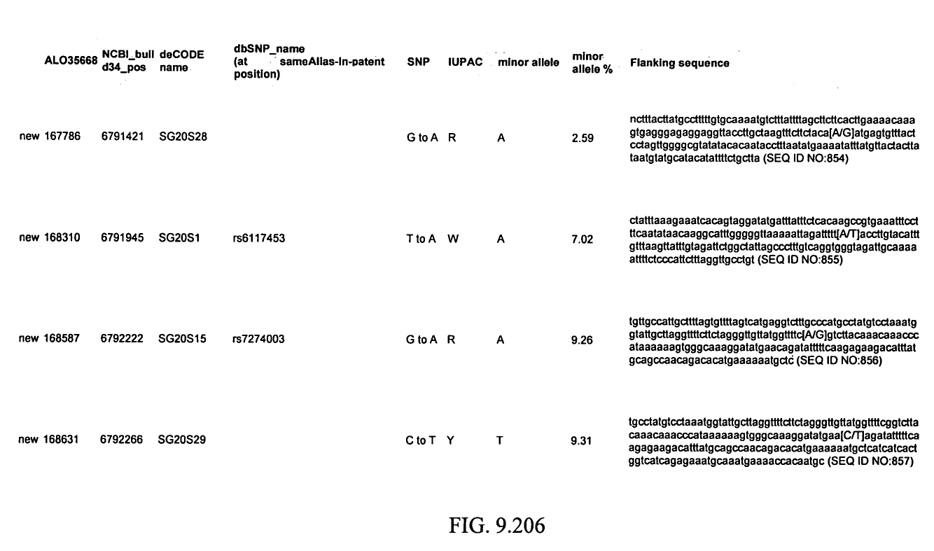

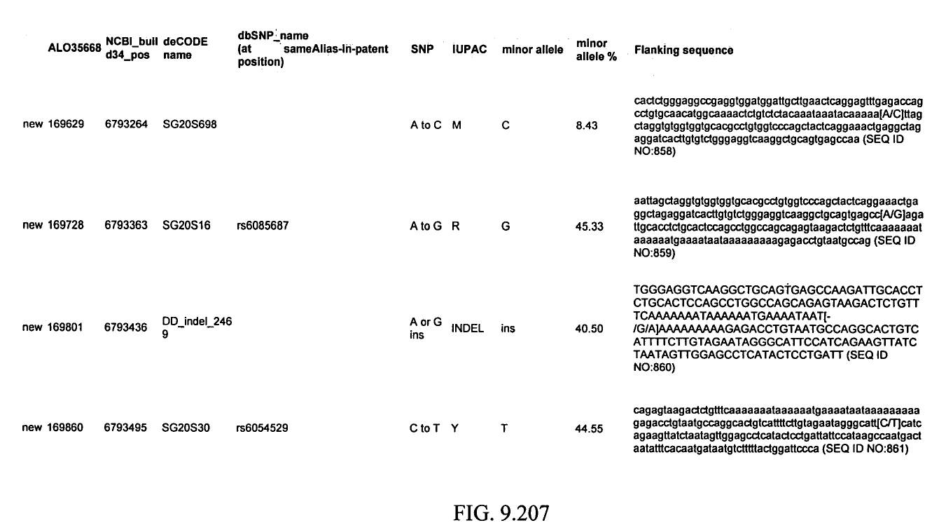

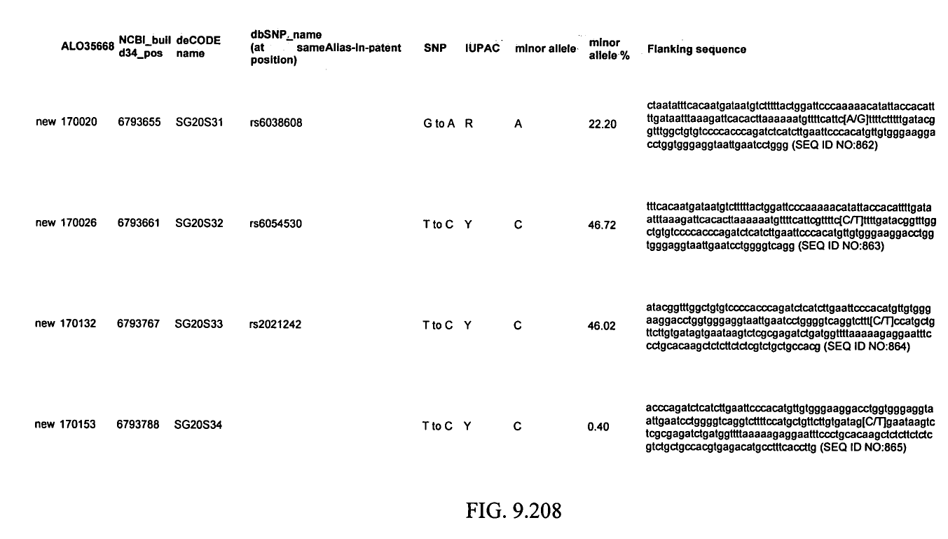

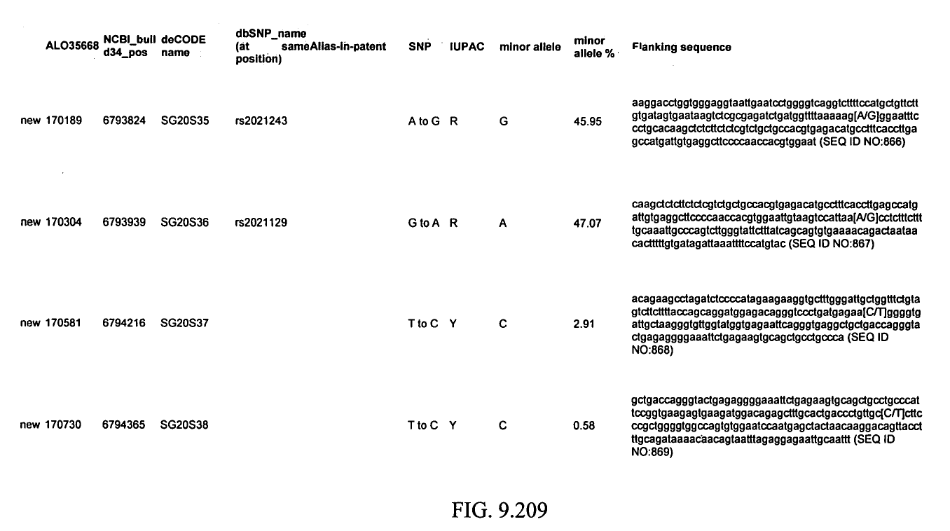

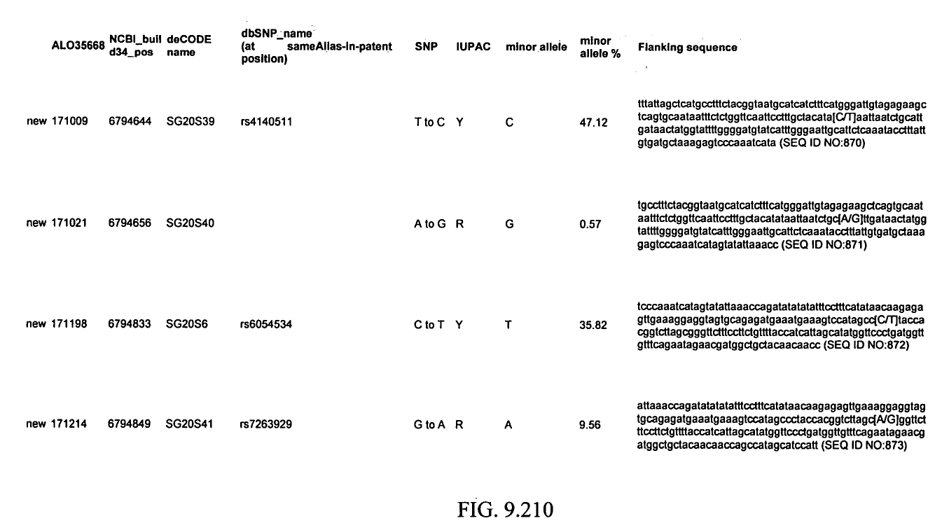

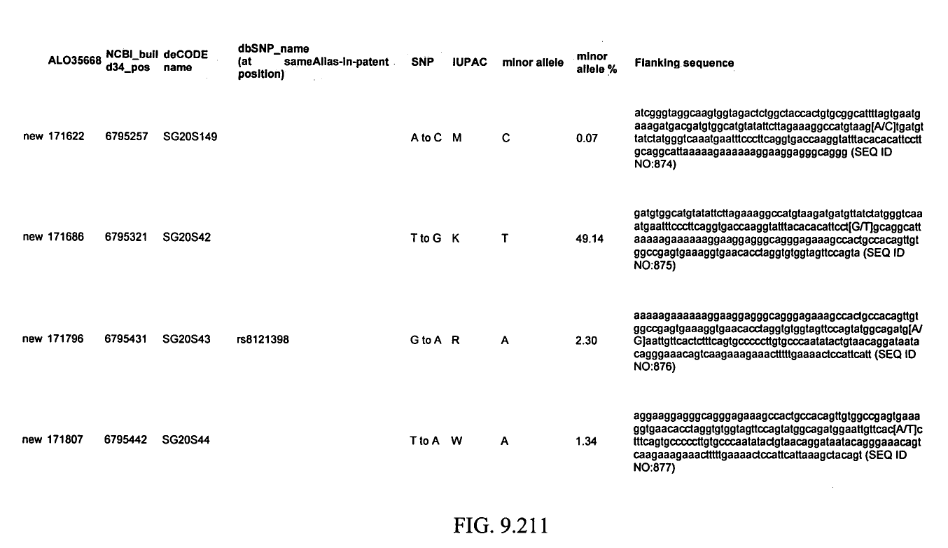

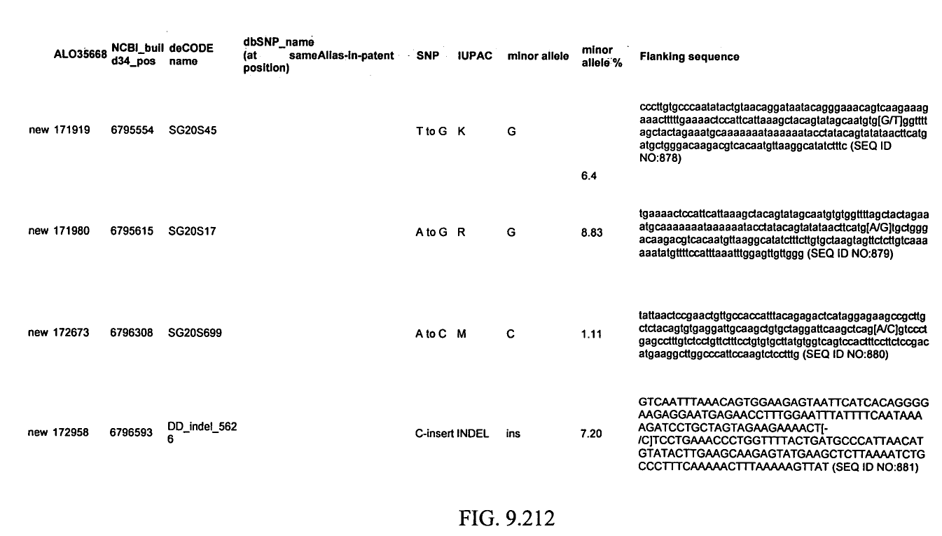

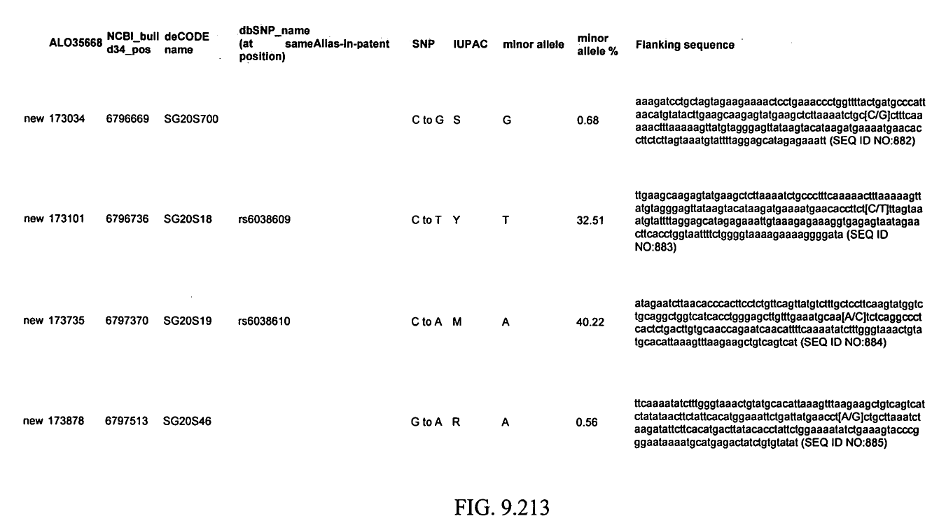

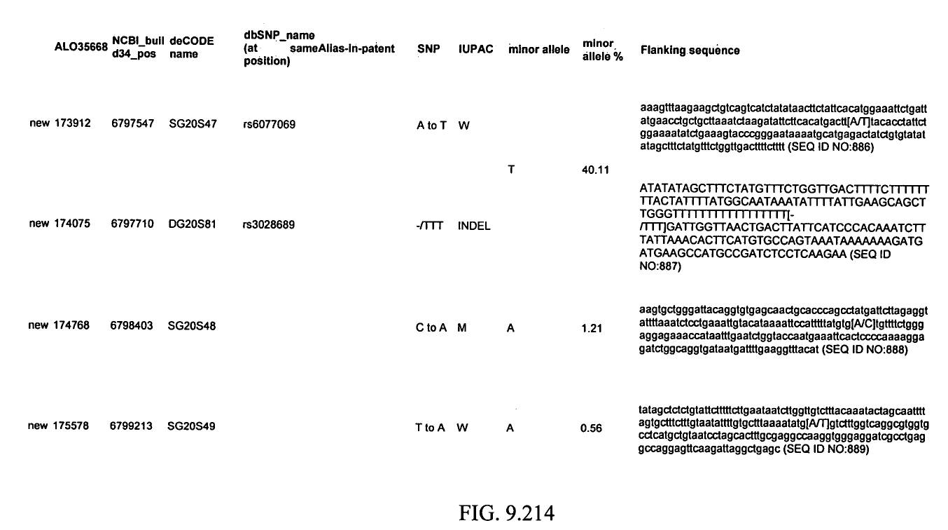

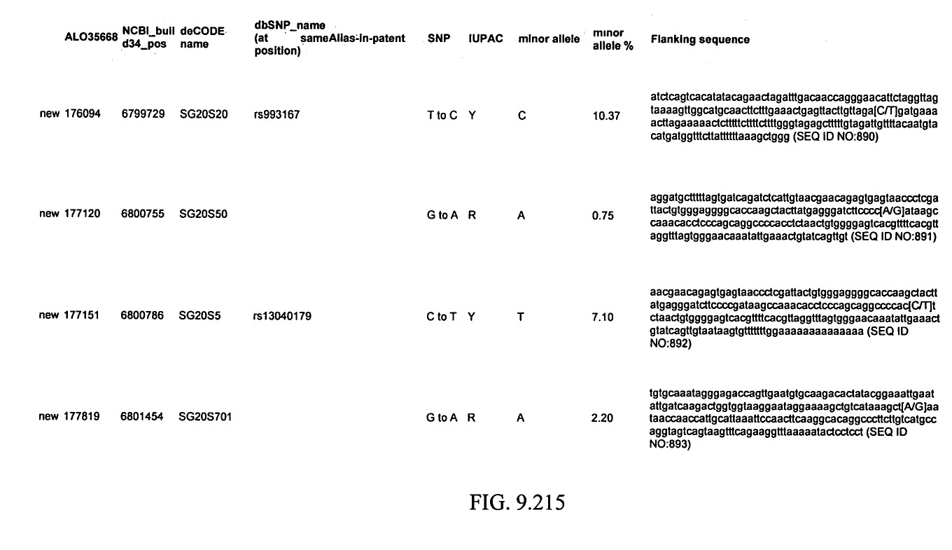

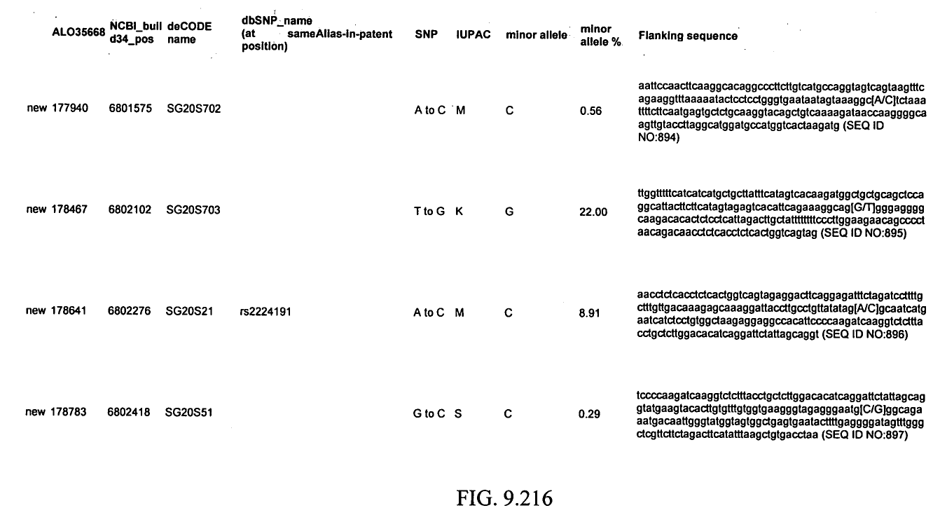

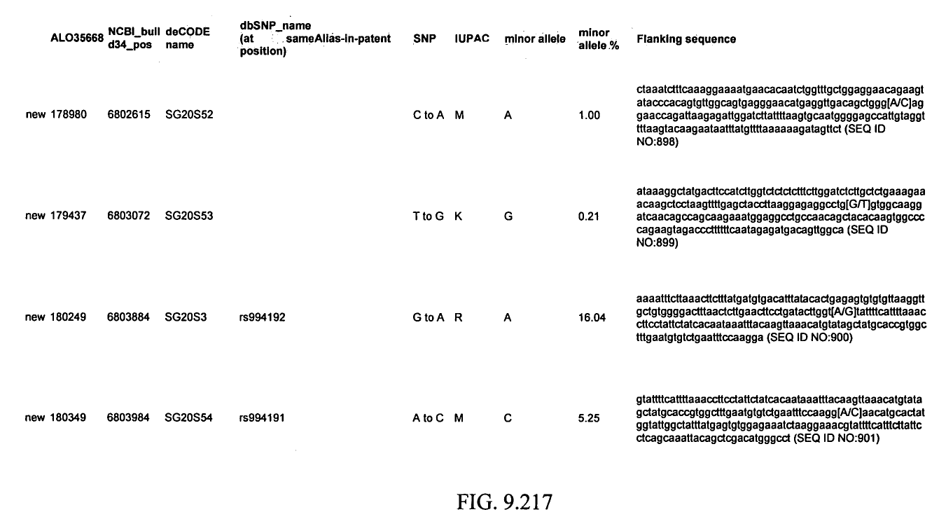

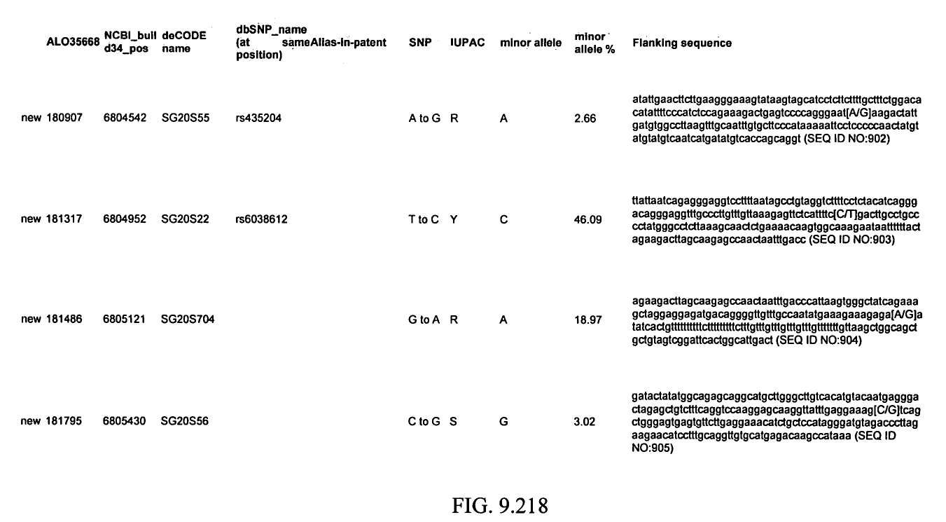

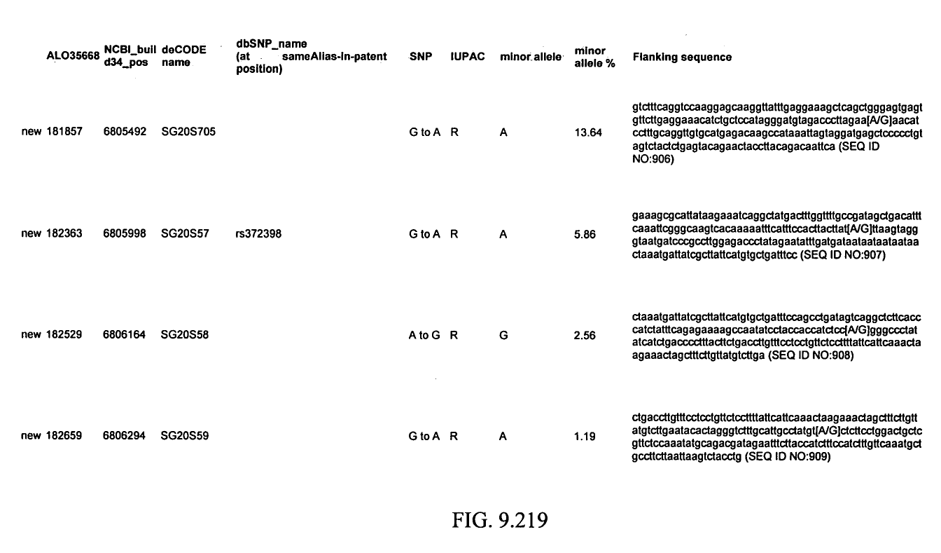

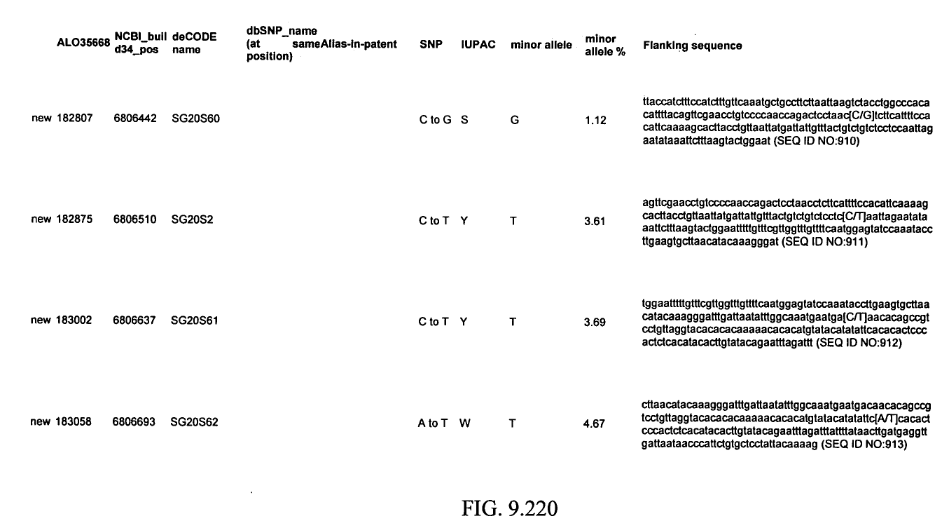

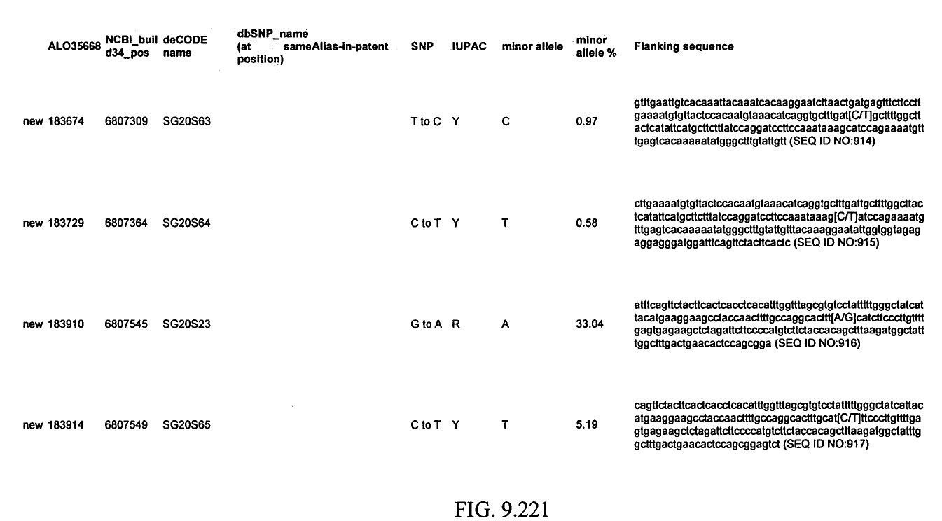

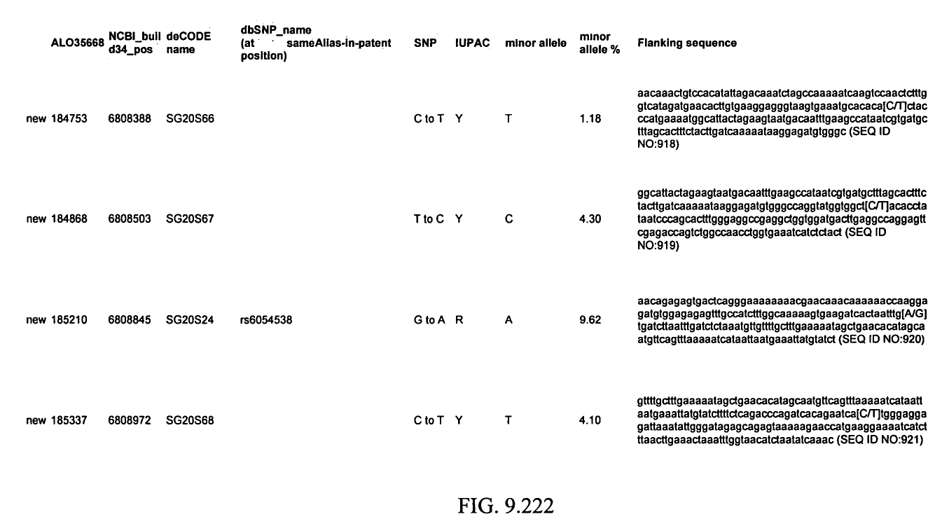

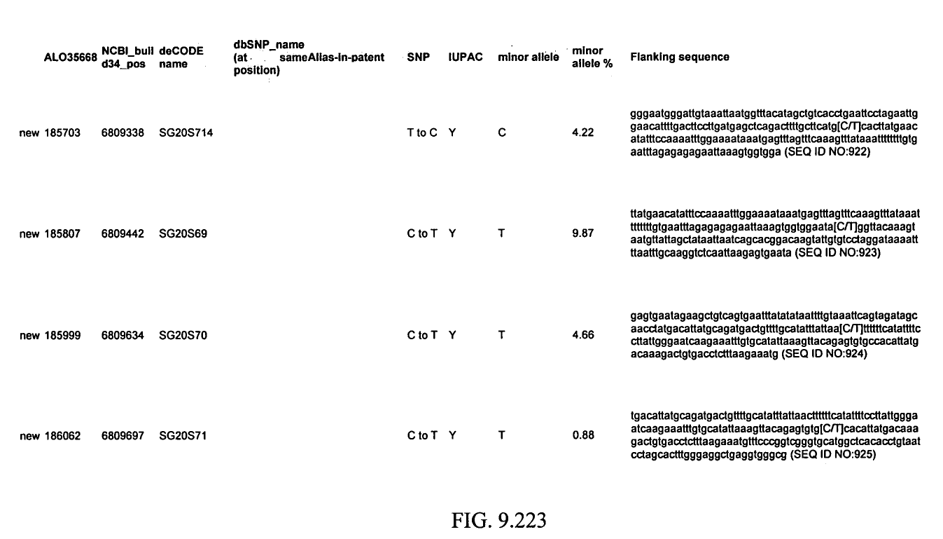

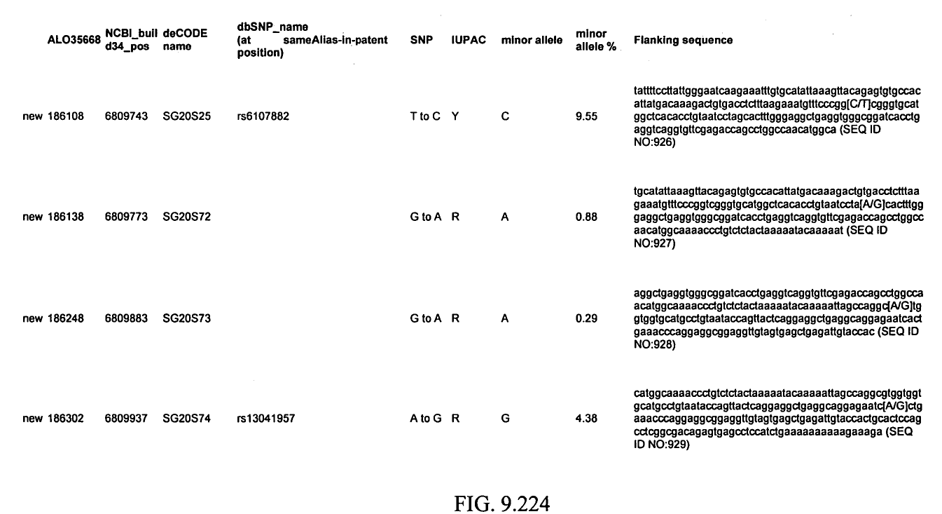

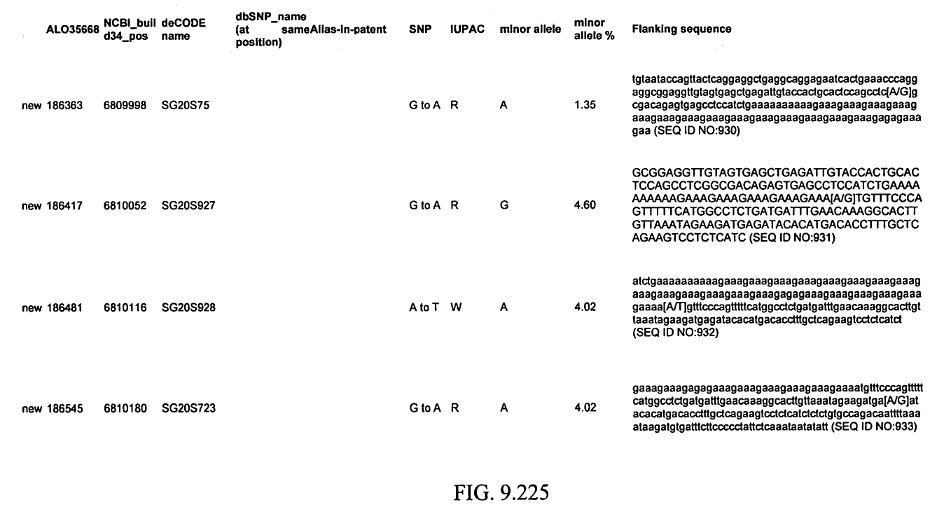

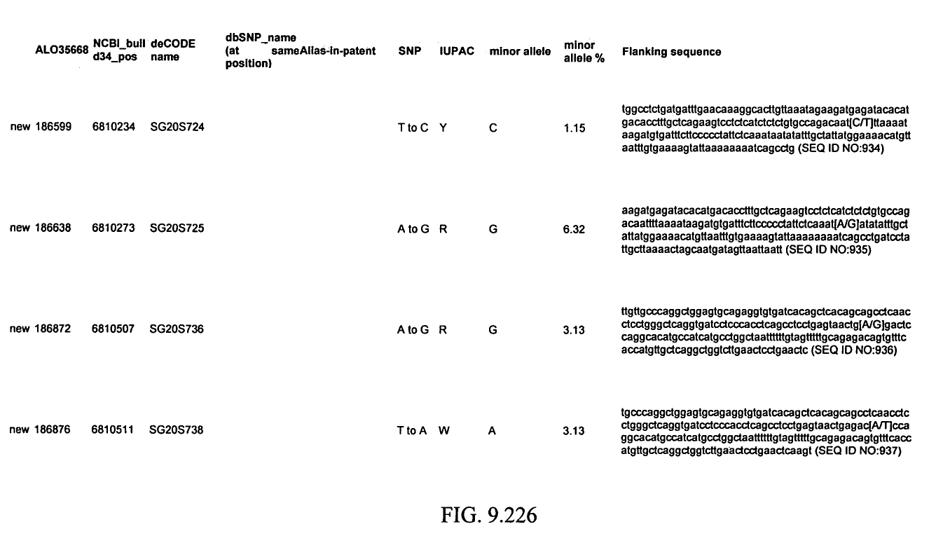

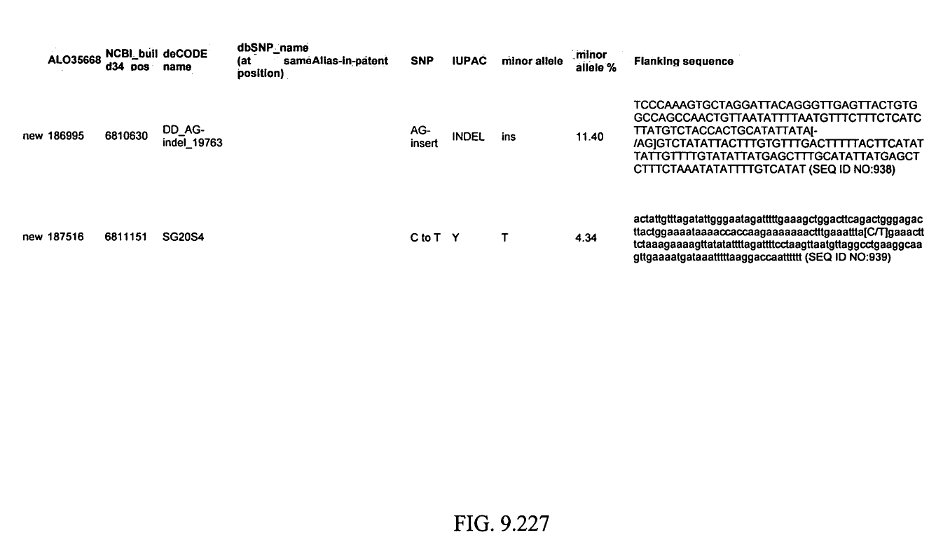

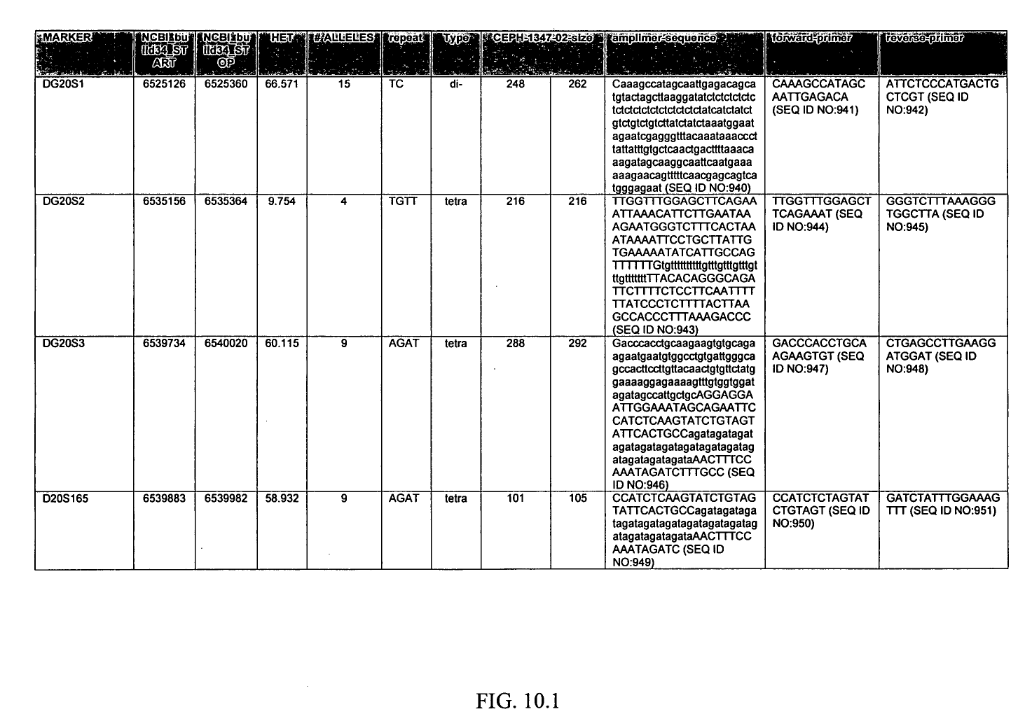

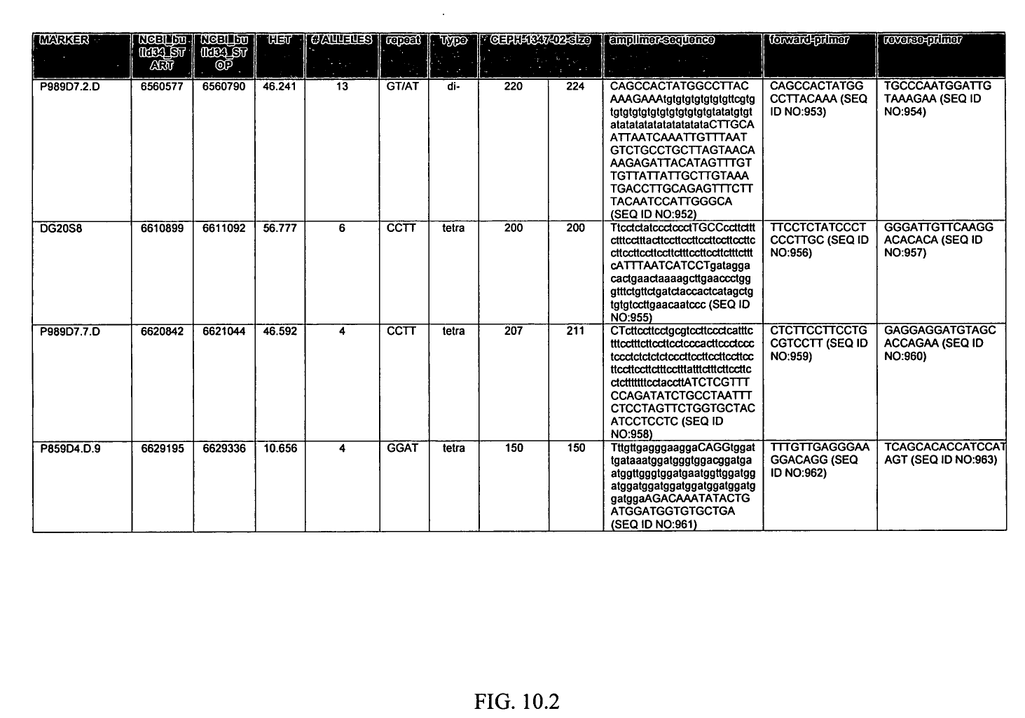

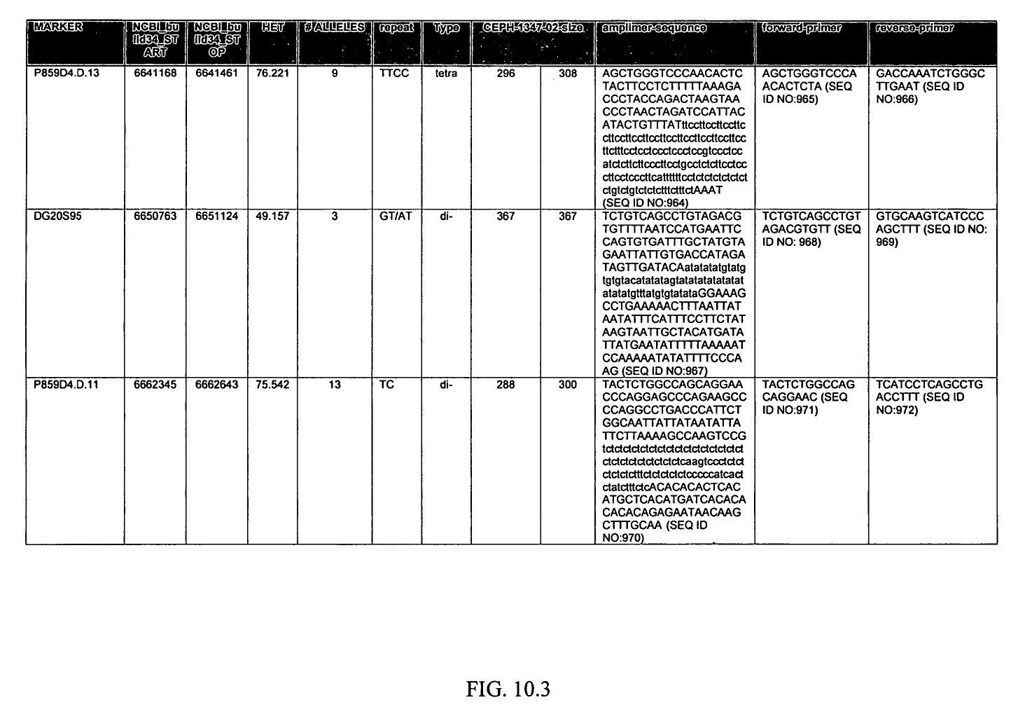

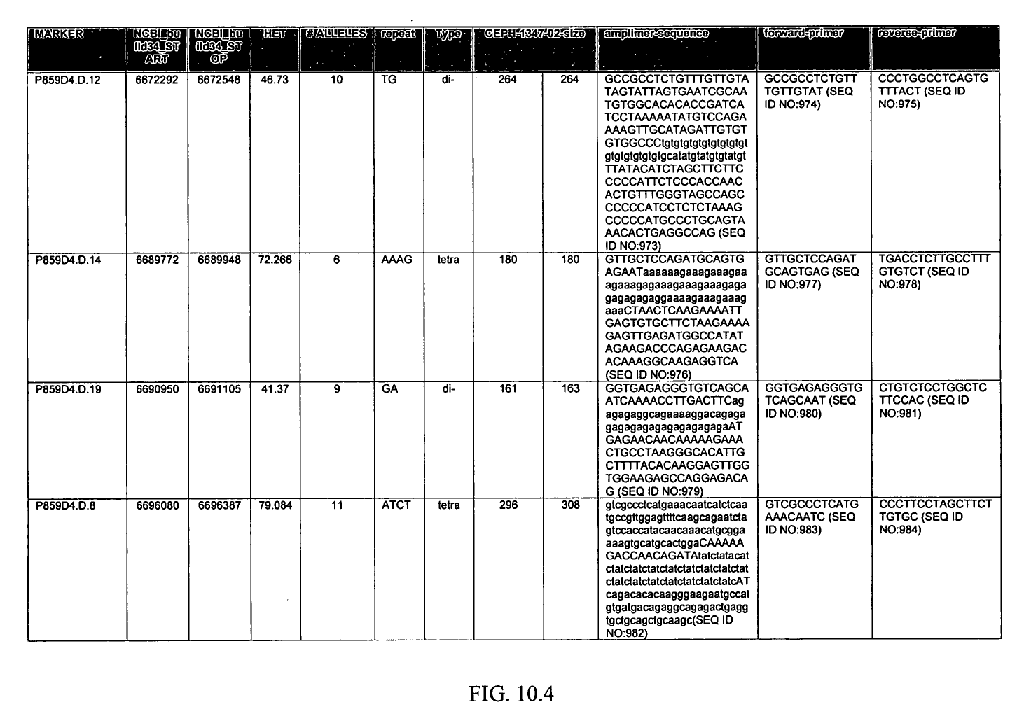

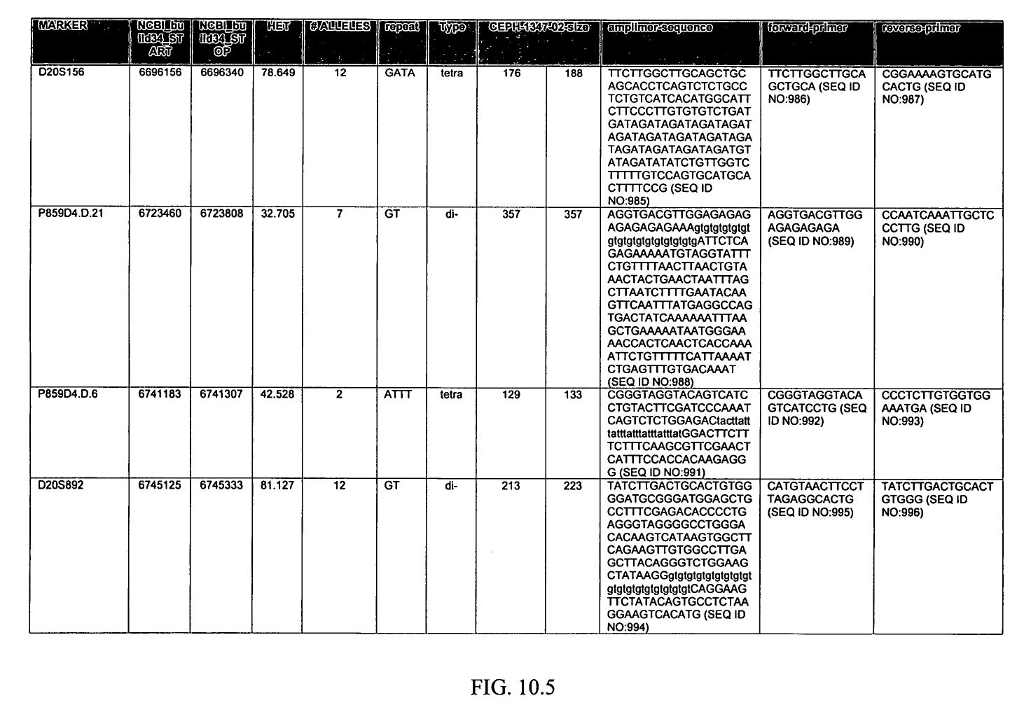

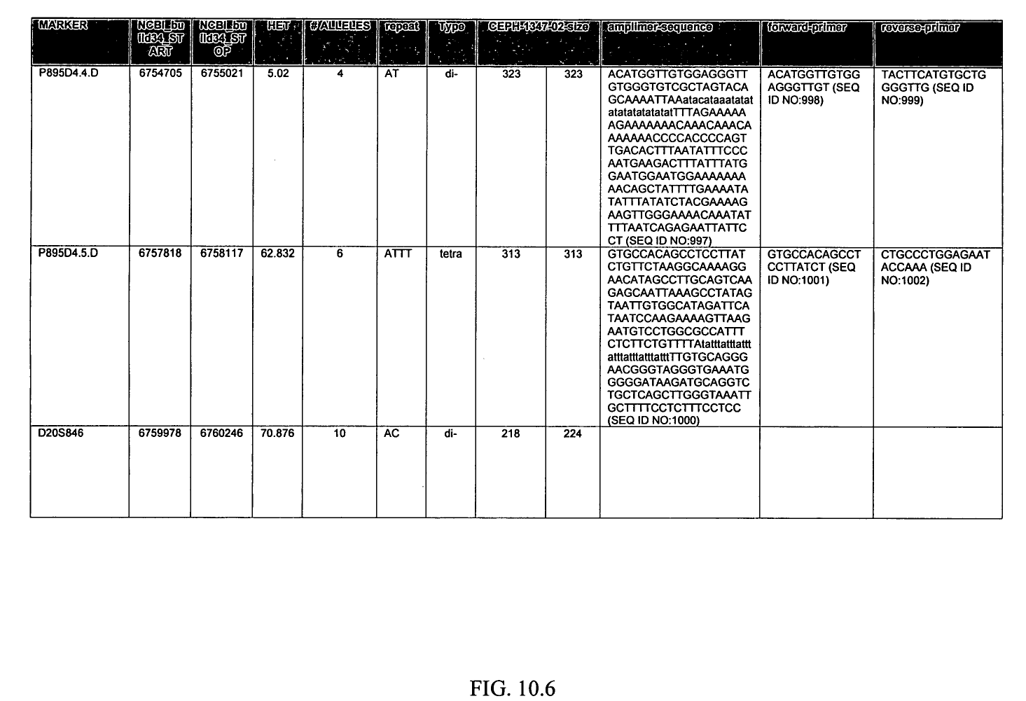

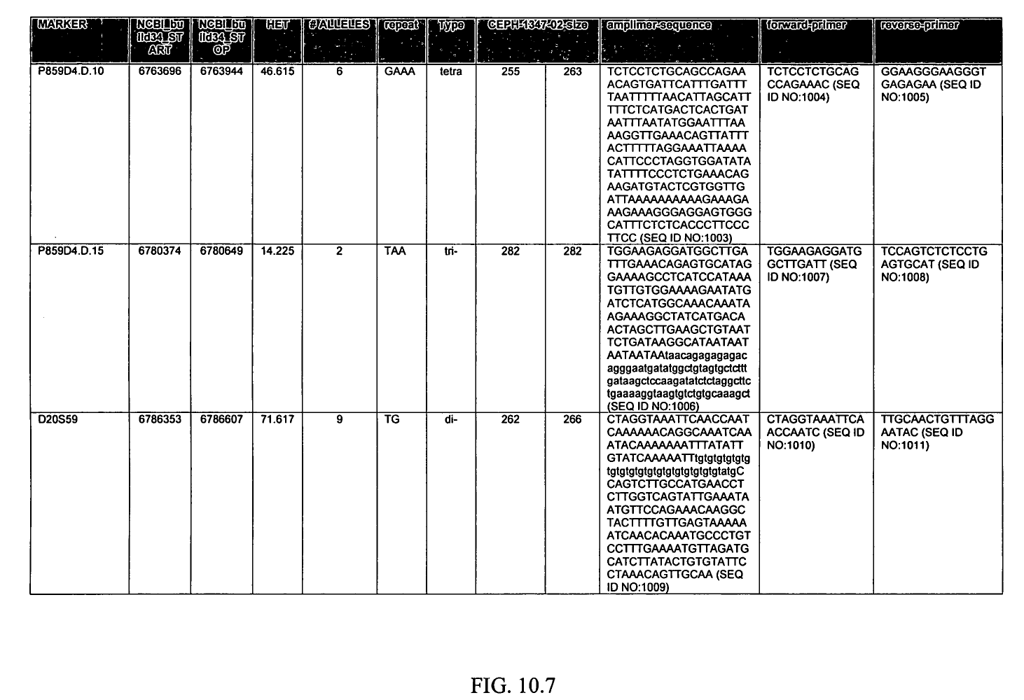

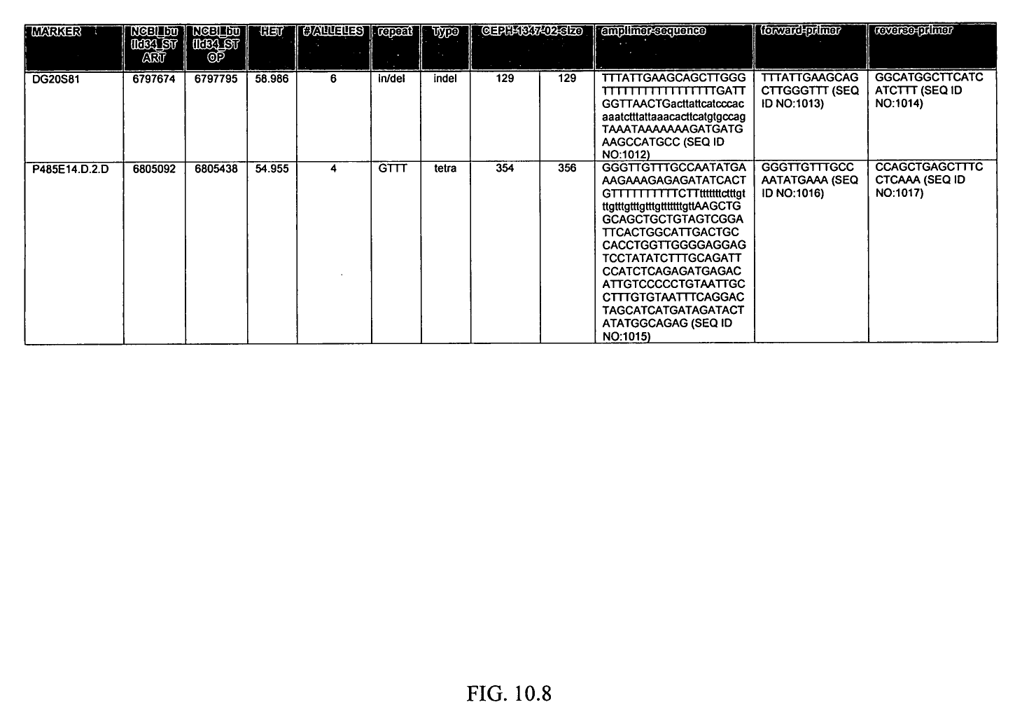

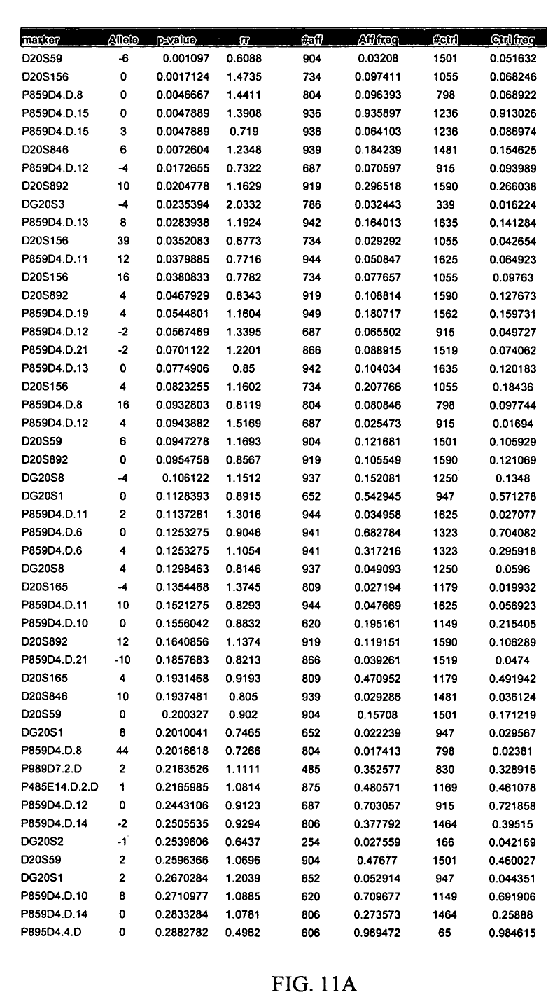

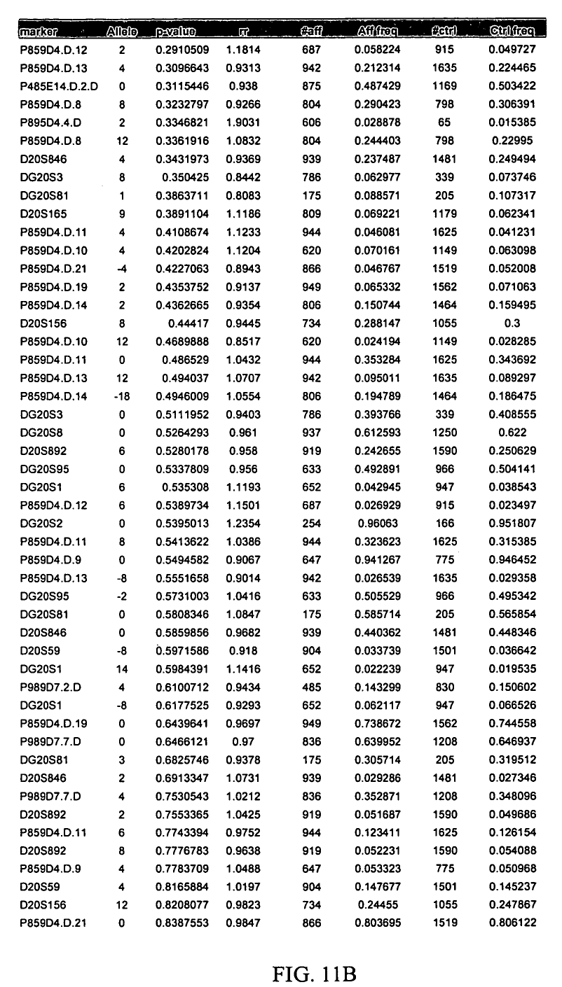

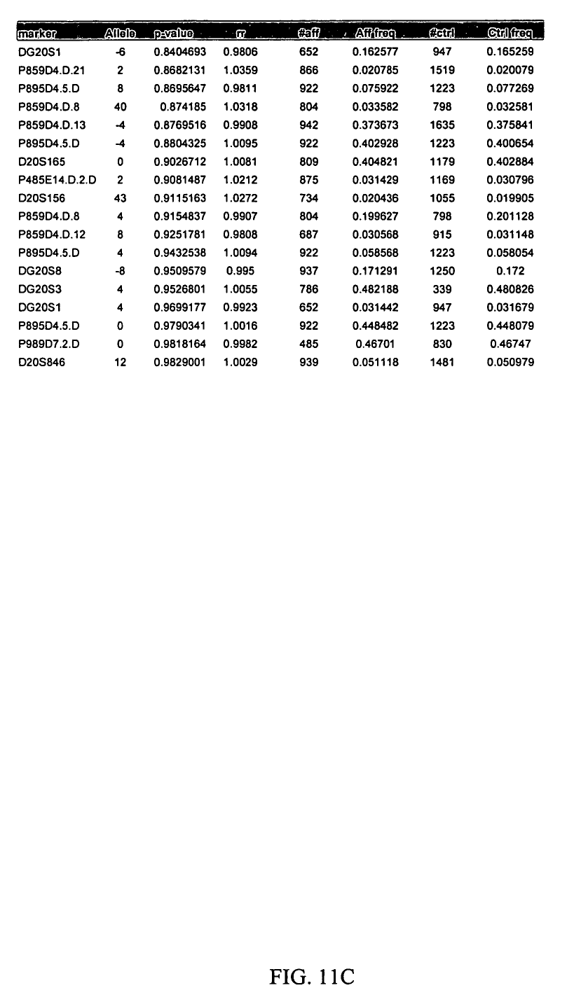

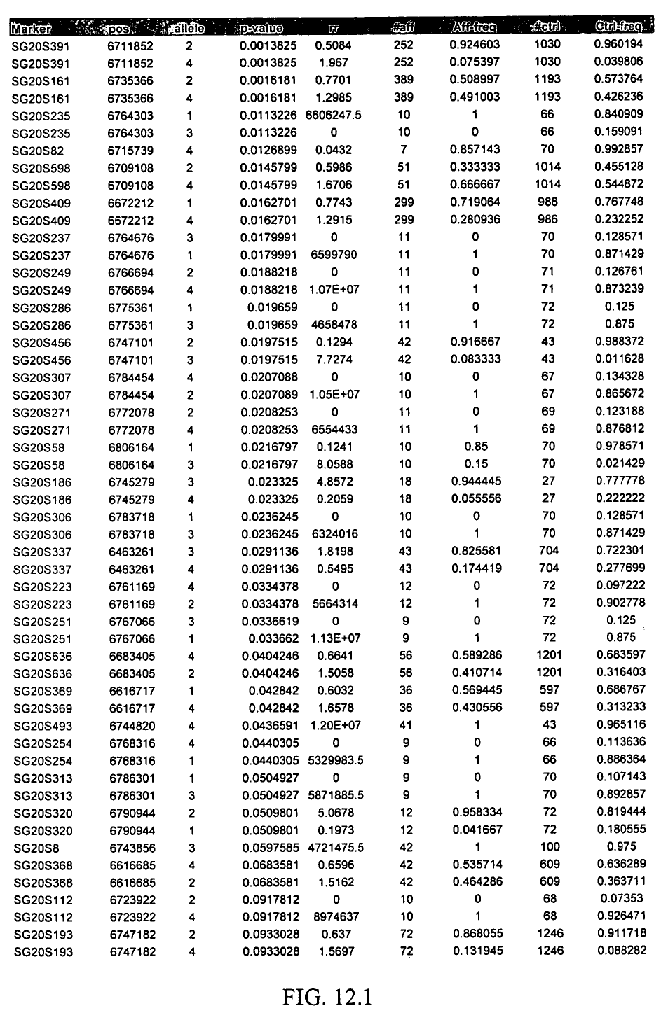

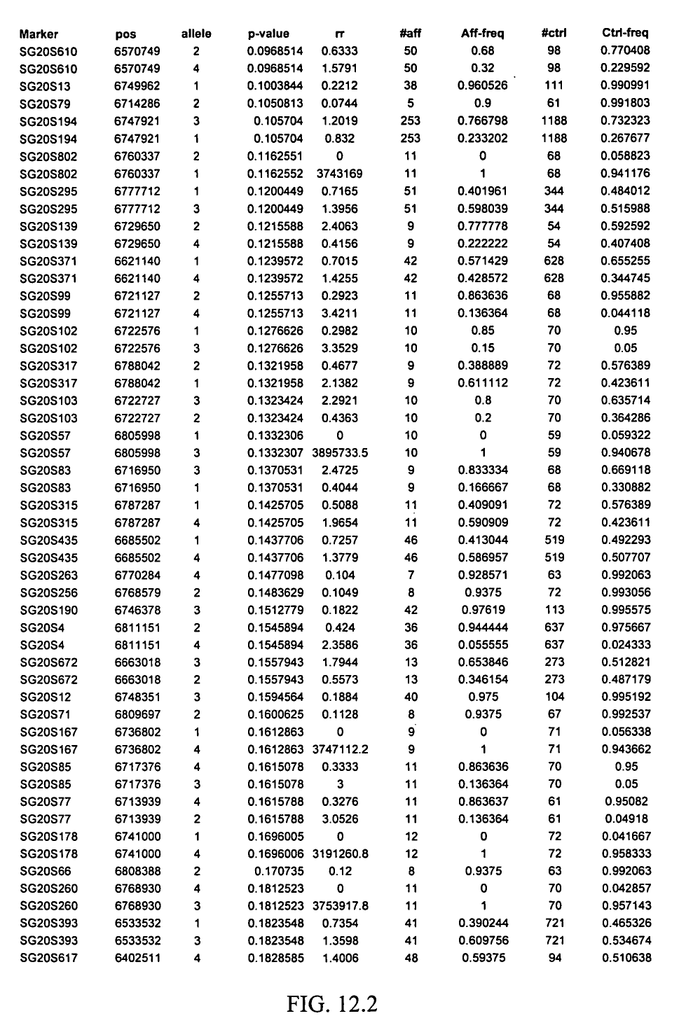

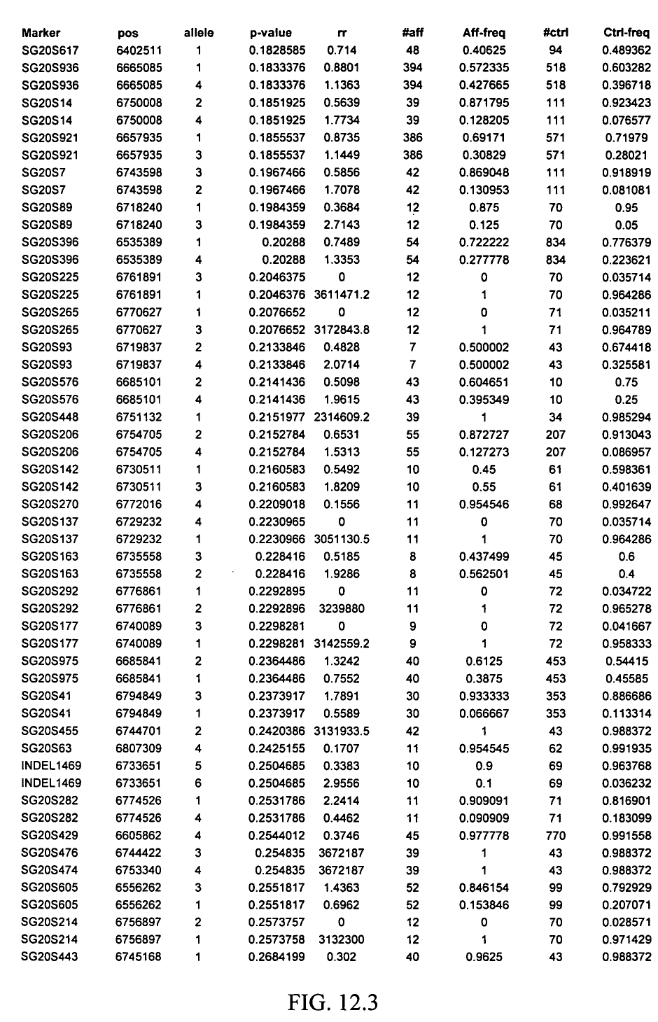

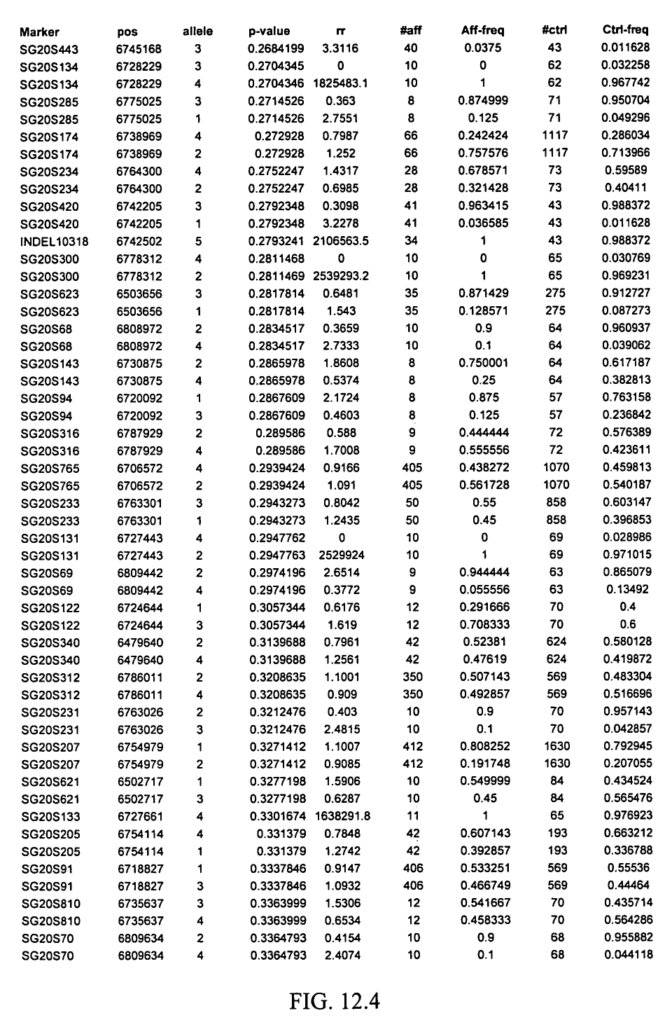

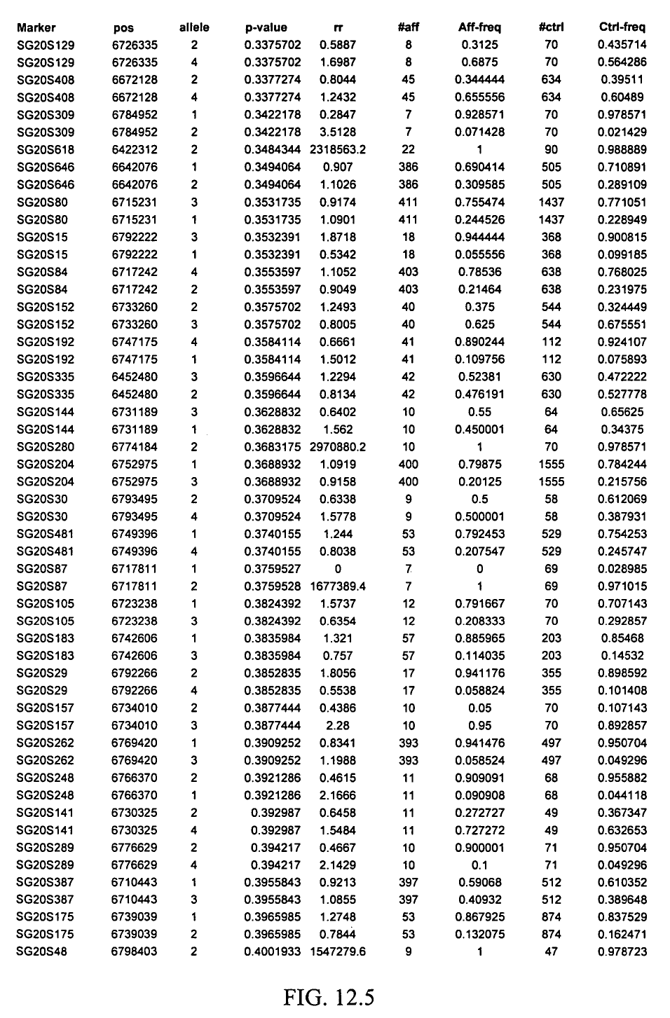

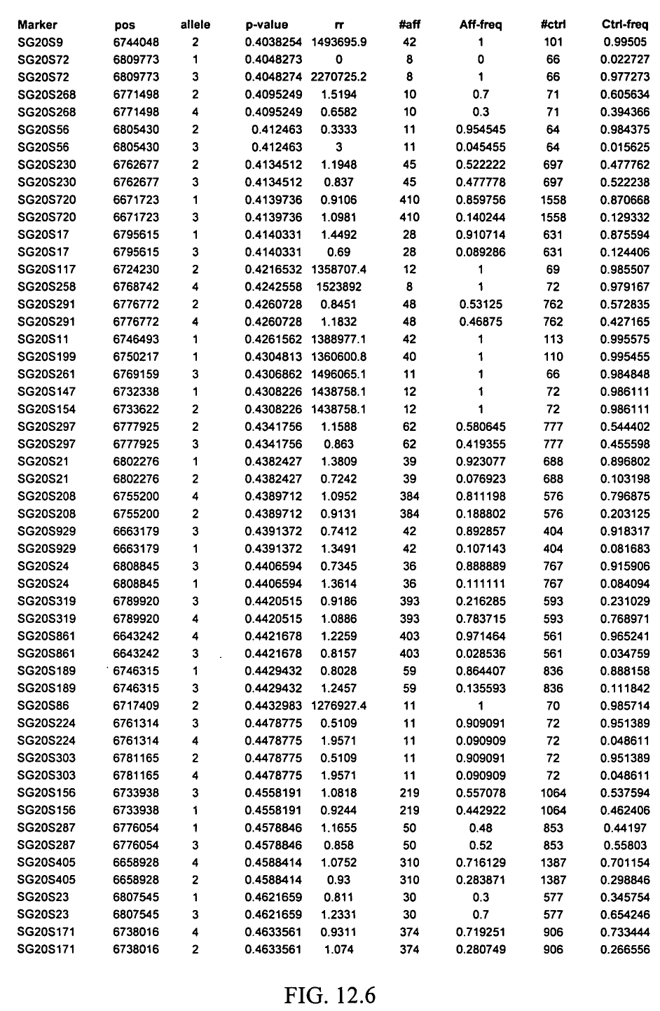

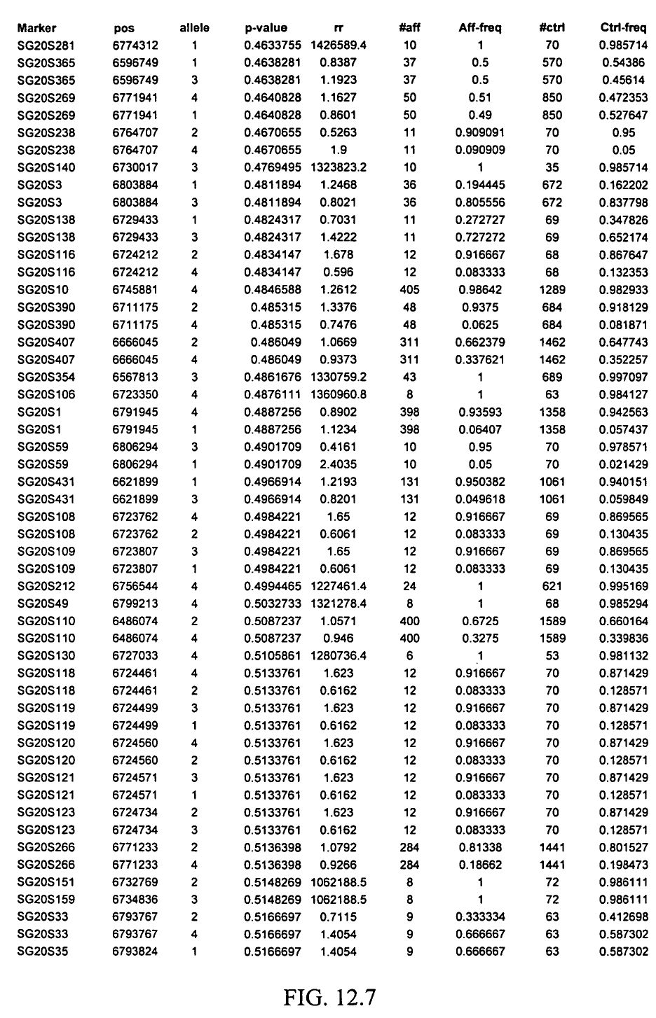

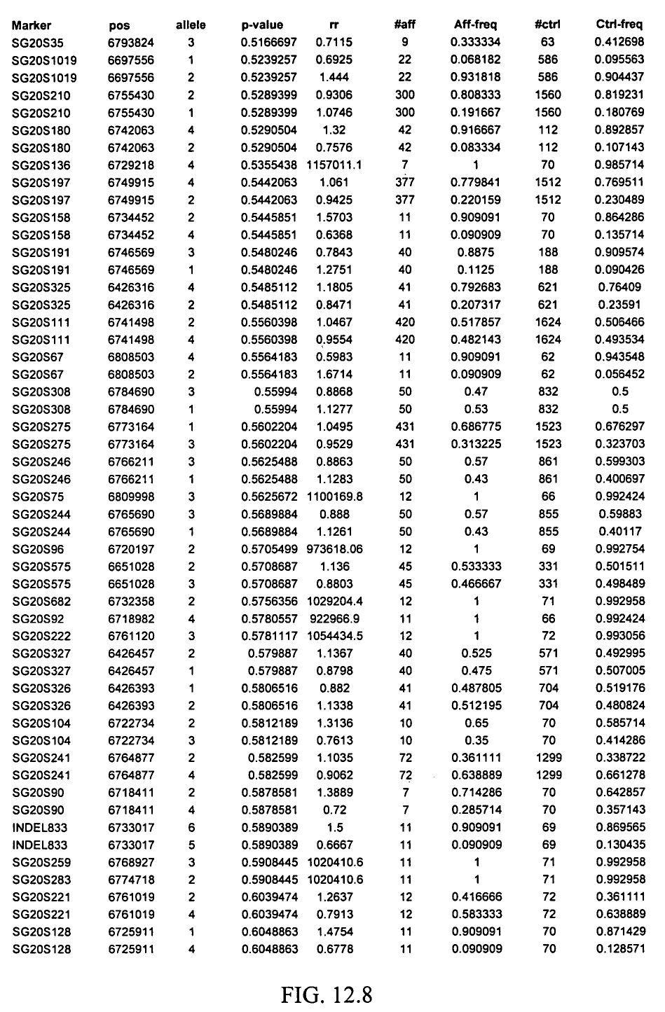

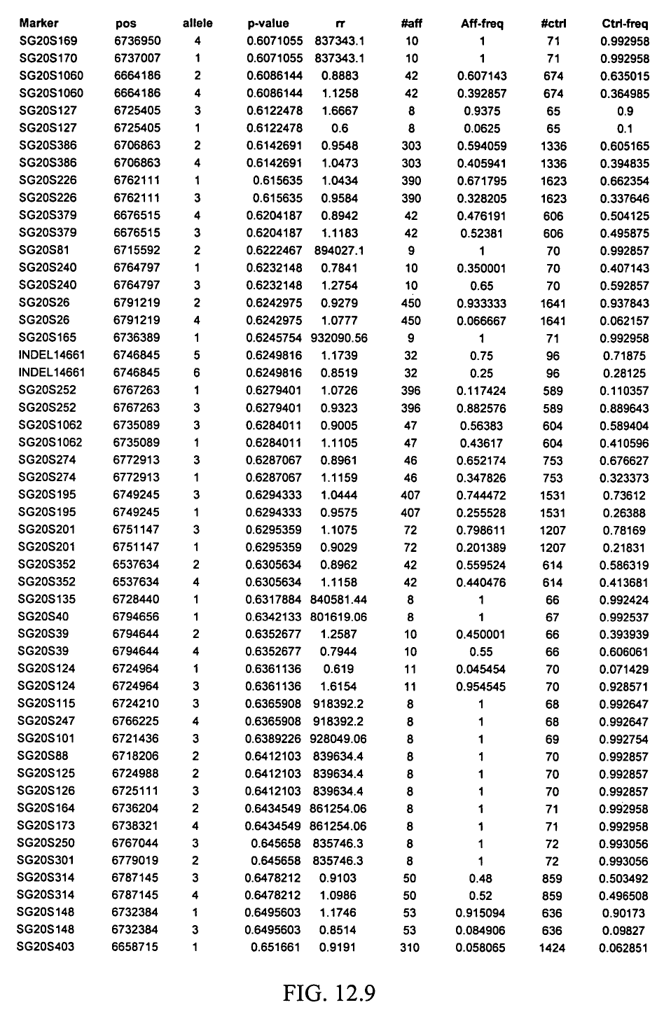

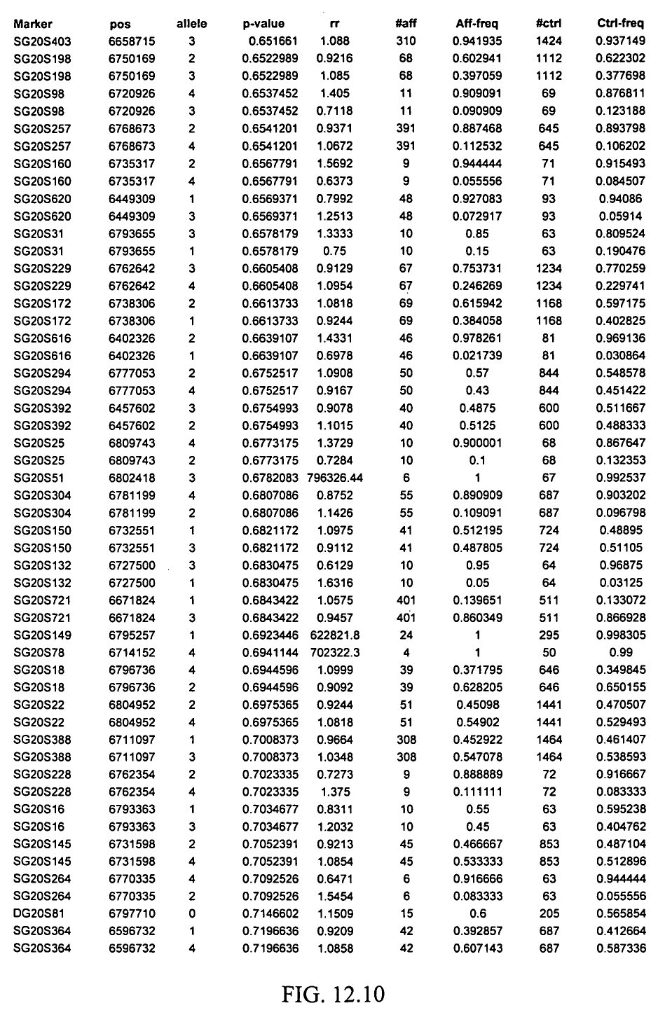

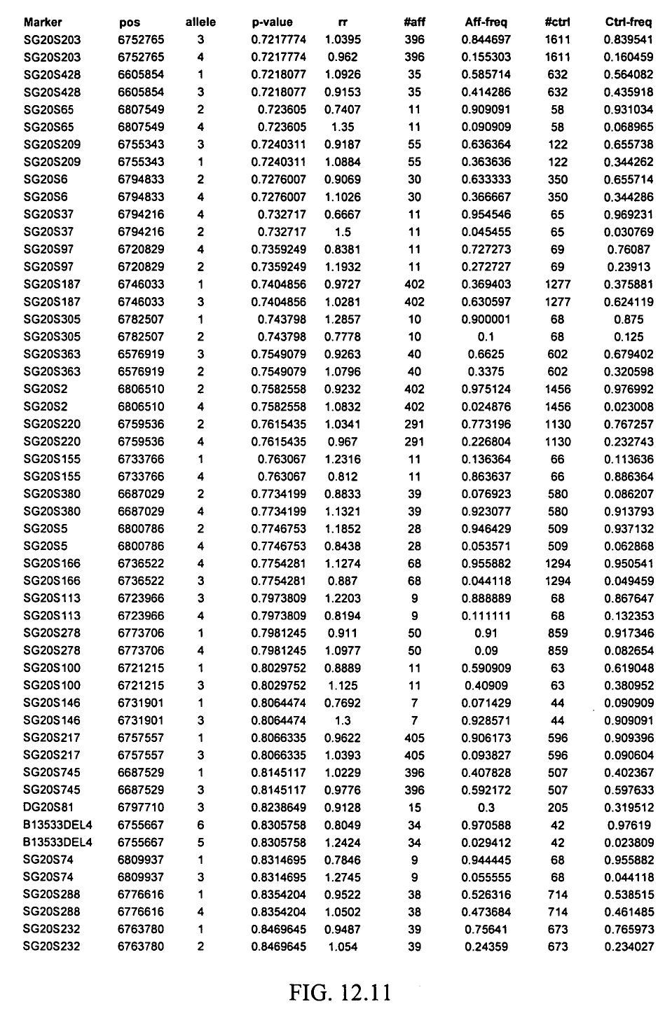

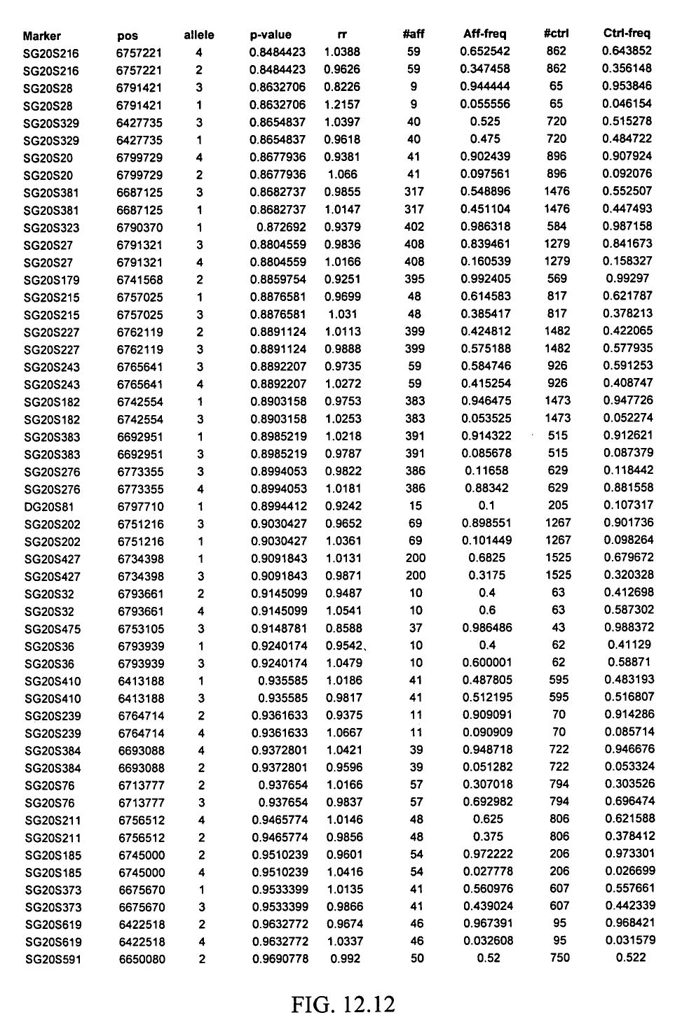

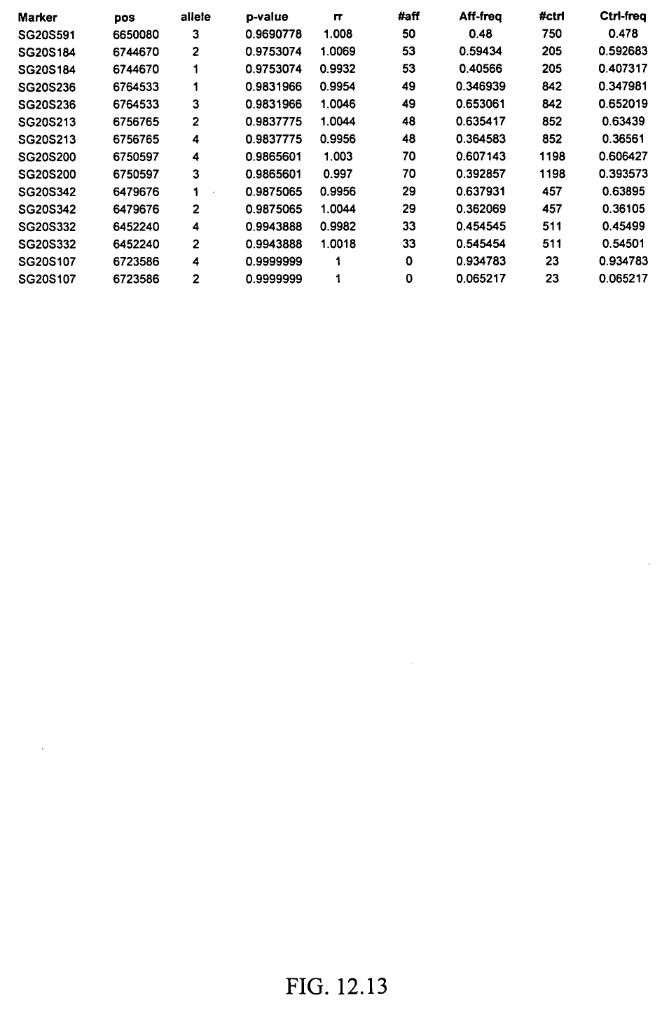

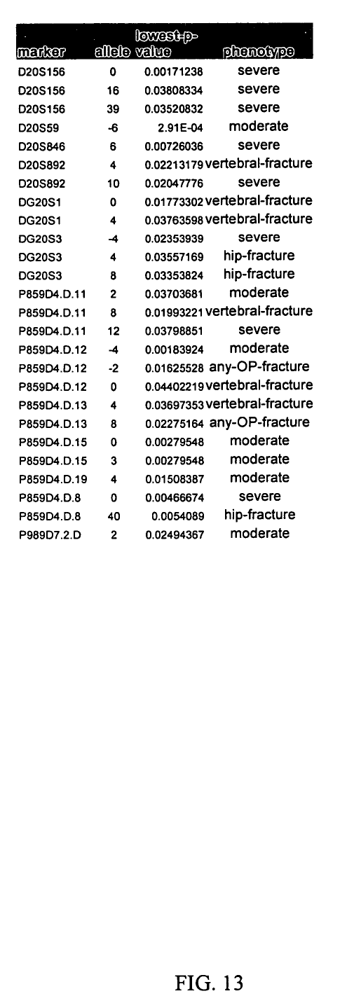

Additional genetic markers were identified in the BMP2 nucleic acid by direct sequencing of the region in different populations. These are listed in Table 4 with nucleotide position relative to Sequence AL035668. Additional markers are listed in FIGS. 9.1-9.227 (SNPs), 10.1-10.8 and 11A-C (microsatellite markers). Associations of markers and osteoporosis-related phenotypes are shown in FIGS. 12.1-12.13 and 13.

TABLE 4

| deCODE | type of | nucleotide position | nucleotide position | | |
|---|---|---|---|---|---|
| numbering | change | relative to SEQ ID NO 1 | relative to AL035668 | location | Public name |
| P4019 | C to T | | 112569 | | |
| P4204 | A to C | | 112754 | | |
| P4337 | T to G | | 112887 | | |
| P4617 | T to A | | 113167 | | |
| P4730 | A to G | | 113280 | | |
| P4765 | T to C | | 113315 | | |
| P4822 | A to G | | 113372 | | |
| P5831 | T to C | | 114381 | | |
| P6121 | A to C | | 114671 | | rs173106 |
| P6136 | A to T | | 114686 | | |
| P6784 | C to T | | 115334 | | rs969643 |
| P6854 | A to C | | 115404 | | |
| P7420 | G to A | | 115970 | | |
| P7904 | A to G | −2047 | 116454 | promoter | |
| P8815 | T to C | −1136 | 117365 | ″ | |
| P9050 | (ATTT)n | −901 | 117600 | ″ | |
| P9313 | C to T | −638 | 117863 | ″ | |
| P9383 | C to T | −568 | 117933 | ″ | |
| P9879 | T to C | −72 | 118429 | ″ | |
| B70 | G to A | 70 | 118570 | ″ | |
| B368 | A insertion | 368 | 118868 | ″ | |
| B420 | A to G | 420 | 118920 | ″ | |
| B472 | A to G | 472 | 118972 | ″ | |
| B1464 | G to C | 1464 | 119964 | 5′ utr | |
| B1722 | G to A | 1722 | 120222 | 5′ utr | |
| B1914 | C to G | 1914 | 120414 | 5′ utr | |
| B2536 | A to C | 2536 | 121036 | intron 1 | |
| B2866 | C to T | 2866 | 121366 | intron 1 | |
| B3145 | G to T | 3145 | 121645 | intron 1 | |
| B3747 | T to G | 3747 | 122247 | exon 2 | rs2273073 |
| B3899 | A to G | 3899 | 122399 | exon 2 | rs1049007 |
| B3918 | G to T | 3918 | 122418 | exon 2 | |
| B4181 | A to G | 4181 | 122681 | intron 2 | |
| B4244 | G to A | 4244 | 122744 | intron 2 | |
| B4359 | A to T | 4359 | 122859 | intron 2 | |
| B4435 | G to A | 4435 | 122935 | intron 2 | |
| B4712 | T insertion | 4712 | 123212 | intron 2 | |
| B5041 | T to A | 5041 | 123541 | intron 2 | |
| B5048 | C to T | 5048 | 123548 | intron 2 | |
| B5787 | G to A | 5787 | 124287 | intron 2 | |
| B6217 | G to A | 6217 | 124717 | intron 2 | |
| B7111 | G to A | 7111 | 125611 | intron 2 | rs235764 |
| B7262 | A to T | 7162 | 125662 | intron 2 | |
| B7781* | T to C | 7781 | 126281 | intron 2 | rs1875274 |
| B7828* | A to G | 7828 | 126328 | intron 2 | |
| B7874* | C to T | 7874 | 126374 | intron 2 | |
| B8035* | G to C | 8035 | 126535 | intron 2 | rs235766 |
| B8083 | A to C | 8083 | 126583 | intron 2 | |
| B8463 | T to G | 8463 | 126963 | intron 2 | rs235767 |
| B9013 | G to A | 9013 | 127513 | intron 2 | rs1005464 |
| B9082 | G to A | 9082 | 127582 | intron 2 | |
| B10631 | G to T | 10631 | 129131 | intron 2 | |
| B10841 | A to G | 10841 | 129341 | intron 2 | |
| B11980 | A to T | 11980 | 130480 | exon 2 | rs235768 |
| B12571 | C to T | 12571 | 131071 | exon 2 | |
| B12845 | A to C | 12845 | 131345 | 3′ utr | rs15705 |
| B13066 | T to C | 13066 | 131566 | 3′ utr | rs3178250 |
| B13209 | A to G | 13209 | 131709 | 3′ utr | rs235769 |
| B13296 | C to A | 13296 | 131796 | 3′ utr | rs170986 |
| B13533del4 | 4 bp deletion | 13533-13536 | 132033 | 3′ utr | |
| D841 | C to T | | 132877 | | |
| D873 | T to C | | 132909 | | |
| D1094 | T to C | | 133130 | | rs235770 |
| D1226 | A to C | | 133262 | | |
| D1354 | G to A | | 133390 | | |
| D1550 | C to T | | 133586 | | TSC0078312/rs28488 |
| D1886 | A to G | | 133922 | | |
| D2048 | C to T | | 134084 | | rs235772 |
| D2269 | C to T | | 134305 | | |
| D2319 | T to A | | 134355 | | |
| D2568 | A to C | | 134604 | | |
| D5348 | C to T | | 137384 | | |
| D5449 | G to A | | 137485 | | |
| D5498 | C to T | | 137534 | | |
| D5643 | G to T | | 137679 | | |
| D6220 | A to G | | 138256 | | rs28151 |
| D6440 | A to G | | 138476 | | |
| D6448 | G to C | | 138484 | | |
| D6683 | C to T | | 138719 | | |
| D6971 | G to T | | 139007 | | TSC0191642/rs910141 |
| D7006 | C to G | | 139042 | | |
| D7355 | C to G | | 139391 | | |
| D7630 | G to A | | 139666 | | |
| D8183 | C to T | | 140219 | | rs235750 |
| D8629 | T to C | | 140665 | | |
| D8632 | A to G | | 140668 | | |
| D8862 | G to A | | 140898 | | |
| D9005 | A to G | | 141041 | | |
| D9036 | C to T | | 141072 | | |
| D9043 | C to T | | 141079 | | |
| D9126 | G to A | | 141162 | | |
| D9206 | T to C | | 141242 | | rs235750 |
| D9473 | T to G | | 141509 | | |
| D9617 | C to T | | 141653 | | |
| D9970 | G to T | | 142006 | | rs235748 |
| D10019 | G to A | | 142055 | | |
| D10402 | T to C | | 142438 | | |
| D10540 | G to A | | 142576 | | |
| D10554 | T to C | | 142590 | | |
| D10699 | C to A | | 142735 | | |
| D11023 | T to C | | 143059 | | |
| D11373 | G to A | | 143409 | | |
| D11395 | A to G | | 143431 | | |
| D11592 | A to G | | 143628 | | |
| D12541 | C to T | | 144577 | | |
| D12645 | A to T | | 144681 | | |
| D12699 | G to A | | 144735 | | |
| D12908 | C to A | | 144944 | | |
| D13002 | T to C | | 145038 | | |
| D13071 | T to A | | 145107 | | |
| D13256 | G to A | | 145292 | | |
| D13259 | G to T | | 145295 | | |
| D13488 | G to A | | 145524 | | |
| D13749 | A to G | | 145785 | | |
| D14613 | T to C | | 146649 | | |
| D14664 | C to T | | 146700 | | |
| D14956 | G to A | | 146992 | | |
| D15562 | C to T | | 147598 | | |
| D15601 | T to C | | 147637 | | |
| D15827 | C to T | | 147863 | | |
| D16270 | A to G | | 148306 | | |
| D16345 | C to T | | 148381 | | |
| D16407 | T to C | | 148443 | | |
| D16595 | C to G | | 148631 | | |
| D17037 | T to C | | 149073 | | |
| D17242 | G to A | | 149278 | | |
| D17493 | A to G | | 149529 | | rs1116867 |
| D17684 | G to T | | 149720 | | |
| D17794 | G to A | | 149830 | | |
| D18035 | A to T | | 150071 | | |
| D18292 | C to A | | 150328 | | |
| D18307 | C to T | | 150343 | | |
| D18513 | C to G | | 150549 | | |
| D18641 | A to G | | 150677 | | |
| D18855 | A to T | | 150891 | | |
| D19047 | C to A | | 151083 | | |
| D19354 | G to A | | 151390 | | |
| D19690 | G to A | | 151726 | | |
| D20383 | A to G | | 152419 | | |
| D20945 | T to A | | 152981 | | |
| D20958 | C to T | | 152994 | | |
| D20961 | C to T | | 152996 | | |
| D21101 | C to T | | 153137 | | |
| D21190 | C to A | | 153226 | | |
| D21354 | G to A | | 153390 | | |
| D21382 | T to C | | 153418 | | |
| D22041 | A to G | | 154077 | | TSC0278787 |
| D22254 | C to G | | 154290 | | TSC0278788 |
| D22326 | C to T | | 154362 | | |
| D22530del6 | del6bp | | 154566 | | |
| D22603 | T to C | | 154639 | | |
| D22641 | C to T | | 154677 | | |
| D22641 | C to T | | 154677 | | |
| D23348 | C to T | | 155384 | | |
| D24843 | G to A | | 156879 | | |
| D25216 | A to C | | 157252 | | |
| D25494 | C to T | | 157530 | | |
| D25528 | T to C | | 157564 | | rs2876039 |
| D25715 | A to G | | 157751 | | |
| D26836 | A to C | | 158872 | | |
| D28047 | G to A | | 160083 | | |
| D28047 | G to A | | 160083 | | |
| D28783 | C to T | | 160819 | | |
| D29019 | G to A | | 161055 | | |
| D29281 | A to C | | 161317 | | |
| D29461 | T to C | | 161497 | | |
| D29569 | C to T | | 161605 | | |
| D30340 | C to T | | 162376 | | |
| D30630 | G to A | | 162666 | | |
| D31474 | G to T | | 163510 | | |
| D31616 | T to A | | 163652 | | |
| D32258 | T to C | | 164294 | | |
| D32371 | A to C | | 164407 | | |
| D33541 | T to C | | 165577 | | |
| D34249 | T to G | | 166285 | | |
| D34699 | A to G | | 166735 | | |
| D35273 | C to A | | 167309 | | |
| D35548 | C to T | | 167584 | | |
| D35650 | G to T | | 167686 | | TSC032068 |

### Example 4

### Novel Splice-Variants and a New Exon in the BMP2 Gene.

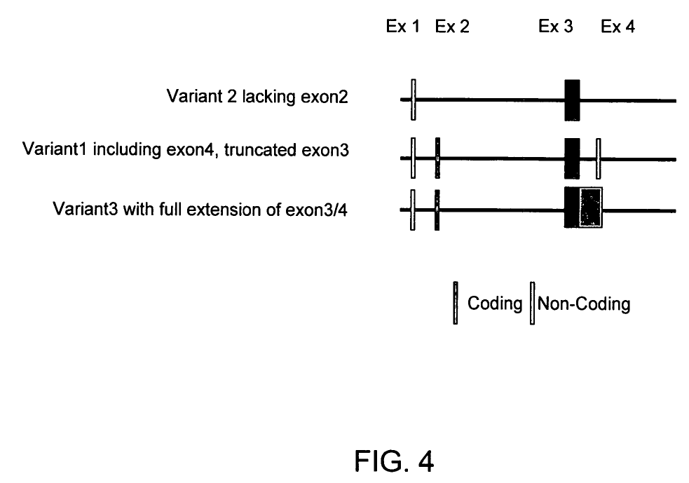

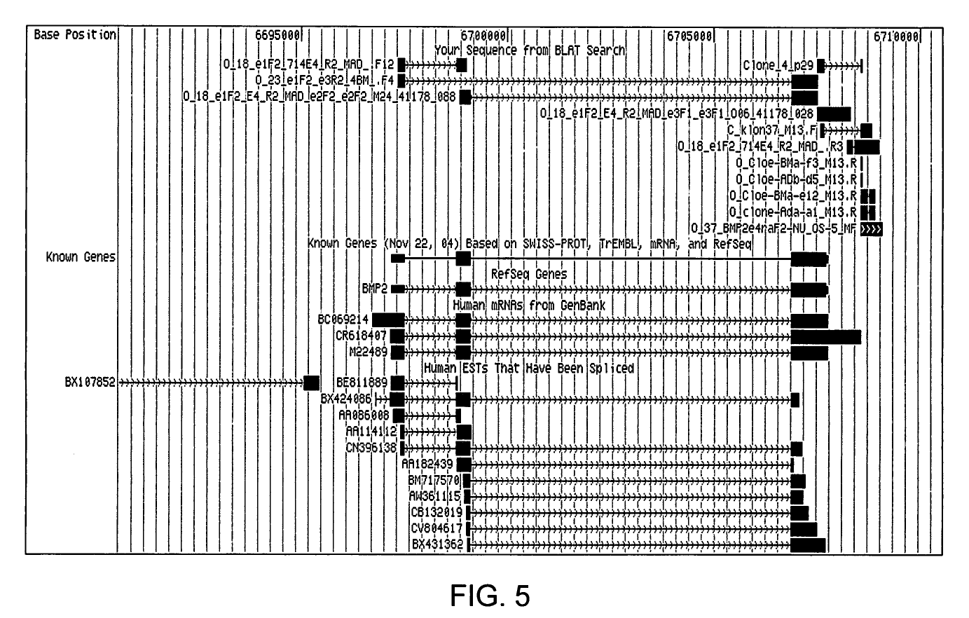

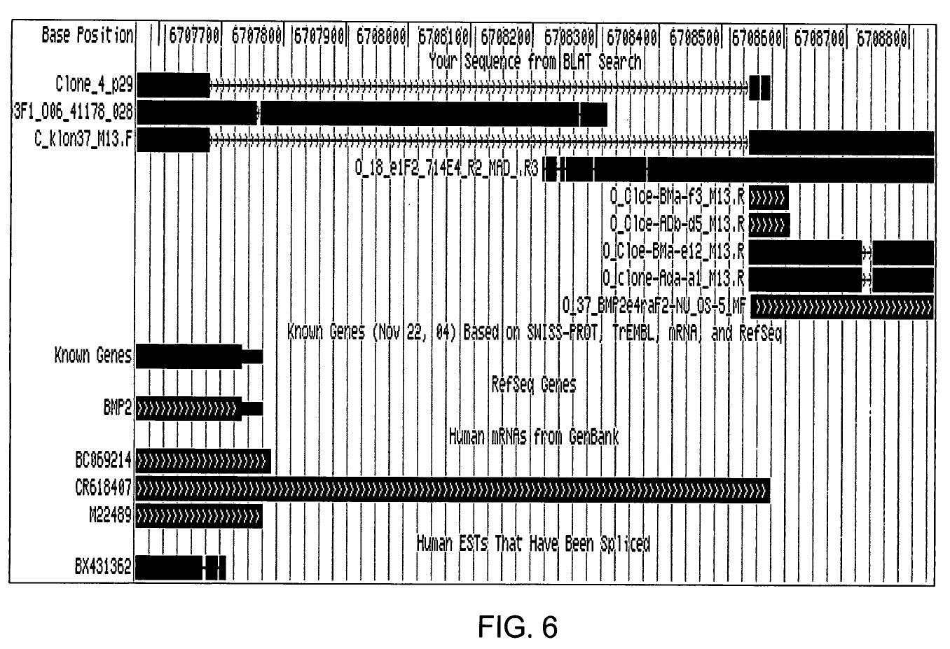

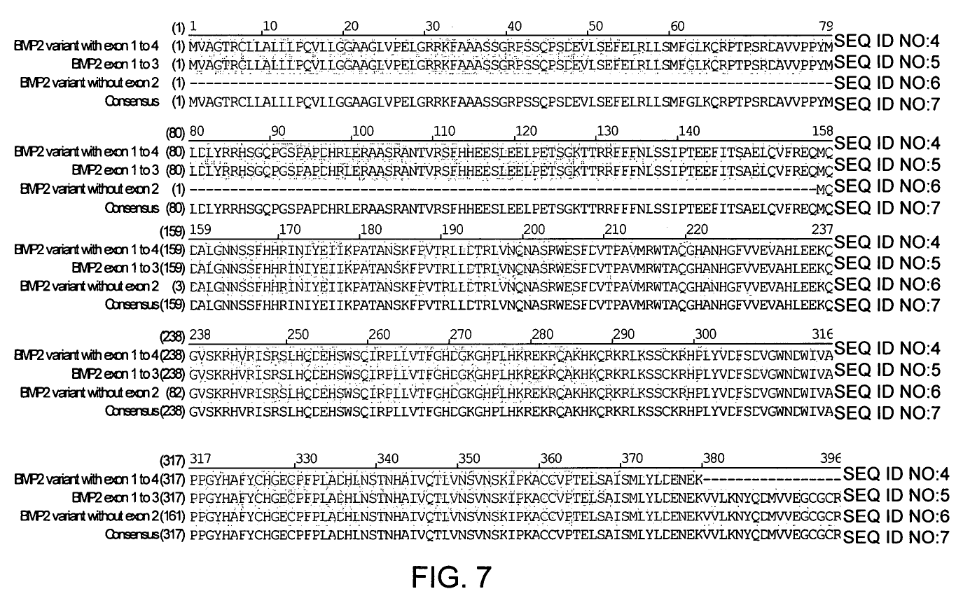

While conducting a search for potential exons in the BMP2 gene, a variable 3′ exon (variant1) and a new splice-variant that excludes exon 2 (variant 2) were identified (see FIG. 4 for a summary of splice site variants in BMP2). Both variants, if translated into proteins, potentially change the amino acid sequence of the BMP2 protein. Furthermore, a variant extending 1315 bp 3′ to the end of BMP2, containing both the exon3 and the newly identified exon as well as the intervening sequence, was also identified (variant3). FIGS. 5 and 6 show clones of variants. An alignment showing the sequences of splice variants and a consensus sequence is shown in FIG. 7.

Procedure:

Known BMP2 exons (NM_{—}001200; Protein: P12643) were connected to 15 putative exons predicted to be transcribed from the same strand such that a primer inside a BMP2 exon and an opposite primer inside the putative exon would result in a positive RT-PCR reaction.

Variant1 and Variant3:

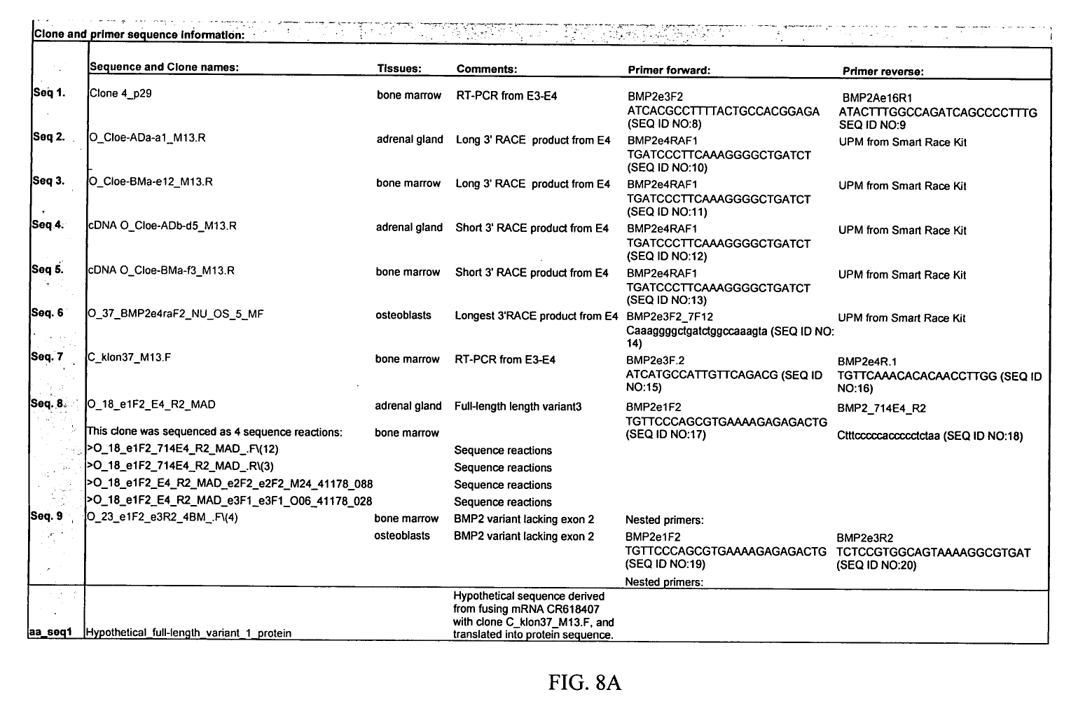

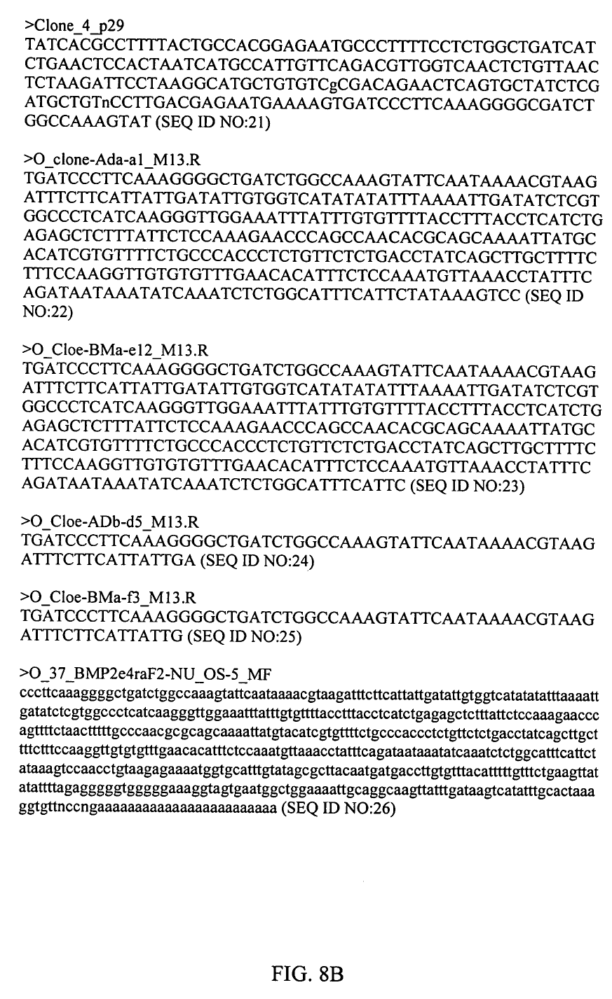

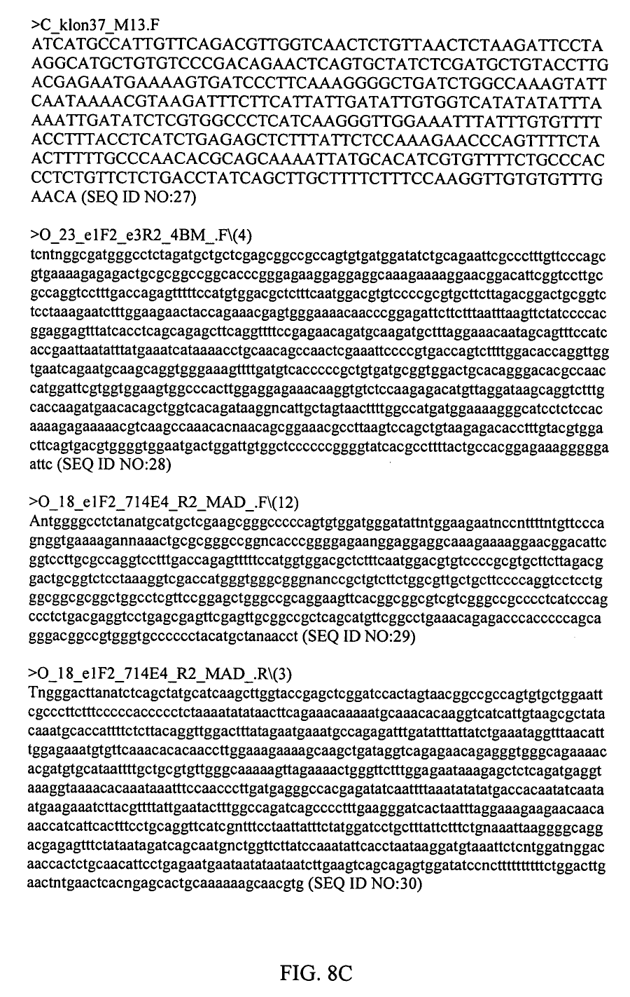

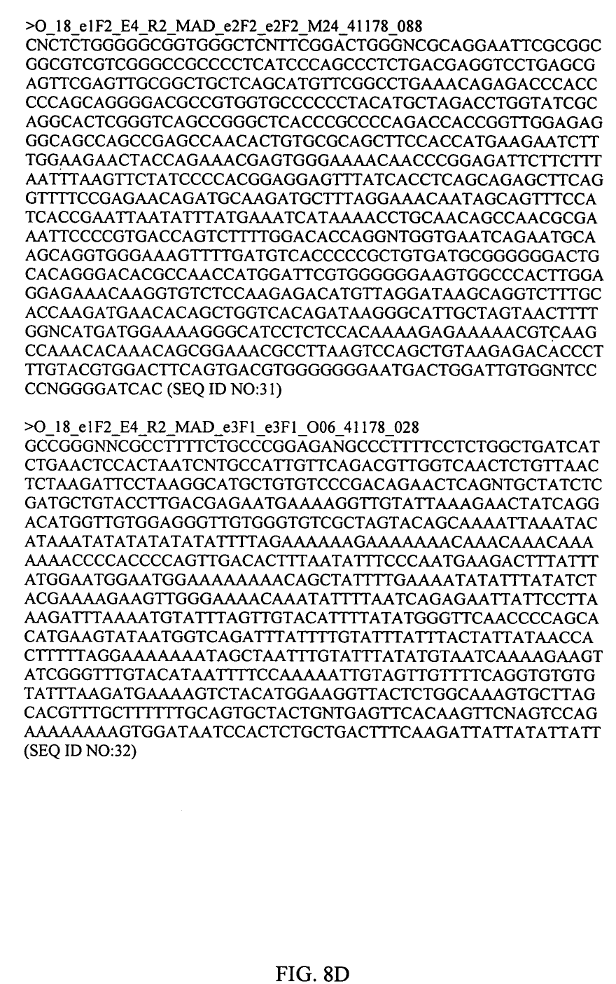

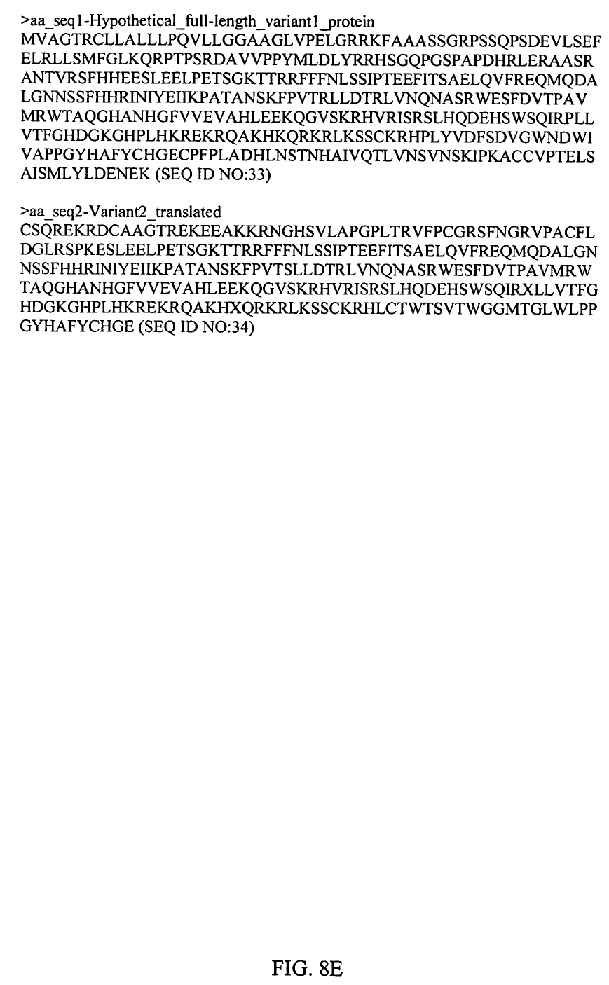

One of these putative exons gave positive results when tested. This alternative 3′ UTR exon (herein referred to as “exon4”) starts 776 bp 3′ to the last known BMP2 exon. It was discovered using bone marrow cDNA, obtained as clone_{—}4_p29 (FIG. 8B). This product connects the new exon to a truncated version of exon3.

Subsequent RACE reactions were set up to characterize the 3′ end of this new exon. Two different sizes of RACE products appeared with both adrenal gland cDNA and with bone marrow cDNA. In an osteoblastic cDNA an even further extension of the exon was obtained (clone O_{—}37_BMP2e4raF2_NU_OS_{—}5_MF, SEQ ID NO:26; FIG. 8B), ending the BMP2 cDNA 1315 bp 3′to the public end (NM_{—}001200).

Confirmation of the existence of this alternative exon4 and the alternative splice site in exon3 was obtained with a RT-PCR reaction in bone marrow cDNA (C_klon37_M13.F, SEQ ID NO:27; FIG. 8C). This new splice variant results in a new and 17aa shorter version of the BMP2 protein.

Exon3 was also shown to have variable 3′ UTR end; the published version, the truncated version connecting to exon4, and an extended version that includes exon4 and the intervening sequence in between (clone O_{—}18_e1F2_E4_R2_MAD, FIGS. 8C and 8D). This extended version results in a 2191 bp size of the last exon of BMP2. The clone was obtained by connecting exon1 to the 3′ end of exon4 in adrenal gland cDNA, and the same variant was also obtained in bone marrow cDNA. The clone was sequenced in parts (FIG. 2 and shown as such on the NCBI_build35 view).

Variant2:

A novel splice variant, which does not include exon2, was detected by RT-PCR connecting exon1 to exon3 in osteoblastic cDNA (hFOB1.19), in adrenal gland cDNA, and in bone marrow cDNA libraries (O23_e1F2_e3R2_{—}4BM_.F(4), SEQ ID NO:28; FIG. 8C). This variant does not include the normal signal peptide or propeptide sequence of the protein because the translational start-site is within exon2. There is an open reading frame starting in exon1 and connecting to the normal frame in exon3, but the first methionine only appears well into exon3. Either an alternative start site is used, which would change the first half of the protein drastically, or, if the first methionine is used, the first half is completely missing. For a description of clone and primer sequences, see FIGS. 8B-E.

Protocols and Programs:

Reverse transcription was performed using Powerscript Reverse Transcriptase (Klondike) and the ThermoScript RT-PCR system (GibcoBRL) according to manufactures protocol. Poly A+ RNA from adrenal gland and bone marrow (Klondike), and total RNA from hFOB 1.19 (a human fetal osteoblastic cell line from ATCC) was used for cDNA synthesis.

Exon4 was amplified from the resultant bone marrow using AmpliTaq (Applied Biosystems), applying a 2 step touchdown PCR protocol: 95° C., for 12 min. and then 10 cycles of (95° C., for 30 sec., 63° C., for 30 sec., 72° C., for 1 min.) followed by 34 cycles of (95° C., for 30 sec., 56° C., for 30 sec., 72° C., for 1 min.) and a final extension step at 72°.

All RACE reactions were performed using the above-mentioned RNAs and the SMART RACE cDNA Amplification kit (Klondike) according to manufactures user manual (PT3269-1). Advantage 2 polymerase mix (Klondike) was used.

The variant lacking exon 2 was amplified by RT-PCR with Advantage 2 polymerase mix (Klondike) with the following protocol: 95° C., for 1 min and then 34 cycles of (95° C., for 30 sec., 58° C., for 30 sec., 68° C., for 3 min) and a final extent ion step at 68° C., for 5 min.

While this invention has been particularly shown and described with reference to preferred embodiments thereof, it will be understood by those skilled in the art that various changes in form and details can be made therein without departing from the spirit and scope of the invention as defined by the appended claims.

Transmitted herewith is a copy of the “Sequence Listing” (sheets 1/209 through 209/209), comprising SEQ ID NOS:1-1,017 in paper form for the above-referenced patent application as required by 37 C.F.R. §1.821(c) and a copy of the “Sequence Listing” in computer readable form as required by 37 C.F.R. §1.821(e). Please insert the attached “Sequence Listing” into the application.

As required by 37 C.F.R. § 1.821(f), Applicant's Attorney hereby states that the content of the “Sequence Listing” in paper form and the computer readable form of the “Sequence Listing” are the same and, as required by 37 C.F.R. §1.821(g), also states that the submission includes no new matter.

Please amend the application as follows:

1. A method of diagnosing osteoporosis or a susceptibility to osteoporosis in an individual, comprising detecting the presence or absence of at least one at-risk haplotype comprising a haplotype selected from the group consisting of: haplotype I, haplotype II, haplotype a, haplotype b, haplotype c, haplotype d and combinations thereof, wherein the presence of the haplotype is indicative of osteoporosis or a susceptibility to osteoporosis:

2. A method for assaying the presence of a first nucleic acid molecule in a sample, comprising contacting said sample with a second nucleic acid molecule comprising the haplotype of claim 1.

3. The method of claim 1, wherein determining the presence or absence of the haplotype comprises 1) enzymatic amplification of nucleic acid from the individual, 2) enzymatic amplification and electrophoretic analysis, 3) restriction fragment length polymorphism analysis, or 4) sequence analysis.

4-20. (canceled)

21. A reagent kit for assaying a sample for the presence of at least one haplotype associated with osteoporosis, wherein the haplotype comprises two or more specific alleles, comprising in separate containers:
a) one or more labeled nucleic acids capable of detecting one or more specific alleles of the haplotype; and
b) reagents for detection of said label.

22. The reagent kit of claim 21, wherein the labeled nucleic acid comprises at least one contiguous nucleotide sequence that is completely complementary to a region comprising at least one specific allele of the haplotype.

23. (canceled)

24. A method for the diagnosis and identification of susceptibility to osteoporosis in an individual, comprising: screening for at least one at-risk haplotype associated with BMP2 that is more frequently present in an individual susceptible to osteoporosis compared to an individual who is not susceptible to osteoporosis wherein the at-risk haplotype increases the risk significantly.

25. The method of claim 24, wherein the significant increase is at least about 20%.

26. The method of claim 25, wherein the significant increase is identified as an odds ratio of at least about 1.2.

27-31. (canceled)

32. A method for diagnosing a susceptibility to osteoporosis in an individual, comprising: obtaining a nucleic acid sample from the individual; and analyzing the nucleic acid sample for the presence or absence of at least one haplotype comprising two or more alleles selected from the group consisting of: TSC0898956, B420, B8463, D20S846, TSC0191642, P4337, D20S892, B5048, B9082, D20S59, B7111/rs235764 B12845/rs15705, P9313, B10631, D35548, rs1116867, TSC0278787, D35548 and TSC0271643, wherein the presence of the haplotype is indicative of susceptibility to osteoporosis.

33. The method of claim 32, wherein the haplotype comprises a) two or more alleles selected from the group consisting of: TSC0898956, B420, B8463, D20S846 and TSC0191642, b) two or more alleles selected from the group consisting of: P4337, D20S892, B5048, B9082 and D20S59, c) B7111/rs235764 or B12845/rs15705, d) two or more alleles selected from the group consisting of: P9313, B10631 and D35548, e) two or more alleles selected from the group consisting of: rs1116867, TSC0278787 and D35548, or f) two or more alleles selected from the group consisting of: TSC0271643, P9313 and B7111.

34-38. (canceled)

39. A method of diagnosing osteoporosis or a susceptibility to osteoporosis in an individual, comprising detecting the presence or absence of at least one at-risk haplotype comprising a haplotype selected from the group consisting of: haplotype G, haplotype V, and combinations thereof, wherein the presence of the haplotype is indicative of osteoporosis or a susceptibility to osteoporosis.

40. A method for assaying the presence of a first nucleic acid molecule in a sample, comprising contacting said sample with a second nucleic acid molecule comprising the haplotype of claim 39.

41. The method of claim 39, wherein determining the presence or absence of the haplotype comprises 1) enzymatic amplification of nucleic acid from the individual, 2) enzymatic amplification and electrophoretic analysis, 3) restriction fragment length polymorphism analysis or, 4) sequence analysis.

42-44. (canceled)

45. A kit for assaying a sample for the presence of at least one haplotype associated with osteoporosis of claim 39, wherein the haplotype comprises one or more specific alleles, and wherein the kit comprises one or more nucleic acids capable of detecting the presence or absence of one or more of the specific alleles, thereby indicating the presence or absence of the haplotype in the sample.

46. The kit of claim 45, wherein the nucleic acid comprises at least one contiguous nucleotide sequence that is completely complementary to a region comprising at least one specific allele of the haplotype.

47-54. (canceled)

55. A method for diagnosing a susceptibility to osteoporosis in an individual, comprising:
obtaining a nucleic acid sample from the individual; and
analyzing the nucleic acid sample for the presence or absence of a haplotype comprising one or more alleles selected from the group consisting of: SG20S405, SG20S407, SG20S381, SG20S171, SG20S174, SG20S195 and D20S846, wherein the presence of the haplotype is indicative of susceptibility to osteoporosis.

56. The method of claim 55, wherein the haplotype comprises one or more alleles selected from the group consisting of: SG20S405, SG20S407 and SG20S381.

57. The method of claim 55, wherein the haplotype comprises one or more alleles selected from the group consisting of: SG20S174, SG20S195 and D20S846.

58. A method of diagnosing a susceptibility to osteoporosis in an individual, comprising detecting at least one polymorphism in a human BMP2 gene of SEQ ID NO:1, wherein the polymorphism is selected from the group consisting of those listed in FIGS. 9.1 through 9.227.

59. The method of claim 58, wherein the polymorphism is detected in a sample from a source selected from the group consisting of: blood, serum, cells and tissue.

60. An isolated nucleic acid molecule comprising the nucleic acid of SEQ ID NO:1 with one or more of the nucleic acid changes selected from the group consisting of those listed in FIGS. 12.1 through 12.13 and 13.
